(19) United States
(12) Patent Application Publication
(10) Pub. No.: US 2024/0277747 A1
(43) Pub. Date: Aug. 22, 2024

(54) Compounds for Treating Genetic Diseases

(71) Applicant: Zikani Therapeutics, Inc., Watertown, MA (US)

(72) Inventors: Roger B. Clark, Lexington, MA (US); Yoshitaka Ichikawa, Laguna Niguel, CA (US); Wesley Francis Austin, Belmont, MA (US); Shuhao Shi, Waltham, MA (US); Wenying Wang, Lexington, MA (US); Xiben Li, Lexington, MA (US); Ivan T. Jewett, Boston, MA (US)

(21) Appl. No.: 18/564,324

(22) PCT Filed: May 31, 2022
(86) PCT No.: PCT/US2022/031565

§ 371 Date: Nov. 27, 2023

Related U.S. Application Data
(60) Provisional application No. 63/194,357, filed on May 28, 2021.

(51) Int. Cl. A61K 31/7052; A61P 11/00; A61P 17/00

(52) U.S. Cl. CPC A61K 31/7052; A61P 11/00; A61P 17/00

(57) ABSTRACT

Provided are 13-membered ribosome targeting compounds that can be used to treat genetic diseases, including genetic diseases that are associated with a premature termination codon mutation or other nonsense mutation. The compounds can induce and/or promote readthrough of the premature termination codon mutation. Also provided are pharmaceutical compositions containing the compounds, methods of using the compounds and processes for making the compounds.

### BACKGROUND

Nonsense mutations are mutations where a stop codon (UAA, UAG or UGA) replaces an amino acid-coding codon, leading to premature termination of translation and eventually to truncated inactive proteins. The Human Gene Mutation Database reports the occurrence of thousands of disease-causing mutations, approximately 12% of which are single point (nonsense) mutations that result in a premature termination codon. (Krawczak M, et al., Hum Mutat. 2000, 15, 45-51.; Mort, et al., M. Hum. Mutat. 2008, 29, 1037-47). Nonsense mutations that result in truncated proteins have been demonstrated to account for many forms of genetic disease including cancer, hemophilia, Tay-Sachs, lysosomal storage disorders or mucopolysaccharidoses such as Hurler Syndrome, Duchenne muscular dystrophy, ataxia telangiectasia, Rett syndrome, various inherited retinopathies, cystic fibrosis, recessive dystrophic epidermolysis bullosa (RDEB), junctional epidermolysis bullosa (JEB), and familial adenomatous polyposis (FAP).

Effective treatments for genetic diseases caused by nonsense mutations remain elusive. As a result, the discovery and development of new compounds effective against nonsense and/or frameshift mutations giving rise to premature termination codons and thus useful for the treatment of genetic diseases and disorders caused by nonsense mutations remains an ongoing unmet need.

### SUMMARY

These and other needs are met by the present invention which is directed to compounds that can be used to treat genetic diseases, including genetic diseases associated with a premature termination codon mutation or other nonsense and/or frameshift mutations. The compounds can induce and/or promote readthrough of the premature termination codon mutation.

In an aspect, what is provided is a method for treating a subject having a genetic disease, comprising: administering a therapeutically effective amount of a compound of formula I:

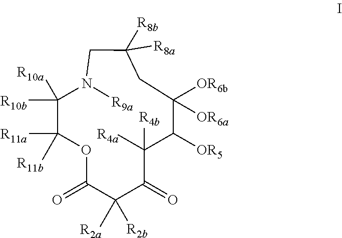

or a pharmaceutically acceptable salt thereof, wherein:
one of R₂ₐ and R_{2b} is selected from the group consisting of H, halo, optionally substituted C₁₋₁₀ alkyl, optionally substituted C₁₋₁₀ alkoxy, and optionally substituted C₂₋₁₀ alkenyl, wherein C₁₋₁₀ alkyl, C₁₋₁₀ alkoxy, and C₂₋₁₀ alkenyl are optionally substituted with one or more groups selected from the group consisting of halo, aryl, amino, alkyl, heteroalkyl, heteroalkenyl, heterocycloalkyl, and heteroaryl; and the other of R₂ₐ and R_{2b} is selected from the group consisting of halo, optionally substituted C₁₋₁₀ alkyl, optionally substituted C₁₋₁₀ alkoxy, and optionally substituted C₂₋₁₀ alkenyl, wherein C₁₋₁₀ alkyl, C₁₋₁₀ alkoxy, and C₂₋₁₀ alkenyl are optionally substituted with one or more groups selected from the group consisting of halo, aryl, amino, alkyl, heteroalkyl, heteroalkenyl, heterocycloalkyl, and heteroaryl;
each of R₄ₐ and R_{4b} is independently selected from the group consisting of H and optionally substituted C₁₋₁₀ alkyl;
R₅ is selected from the group consisting of H, a hydroxyl protecting group, and

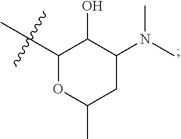 [SMILES: *C1OC(C)CC(N(C)C)C1O]

R₆ₐ is optionally substituted C₁₋₁₀ alkyl;
R_{6b} is H, C₁₋₁₀ alkyl, C₁₋₁₀ hydroxyalkyl, allyl, haloalkyl, aryl, heteroalkenyl, heterocycloalkyl, or heteroaryl, any of which can be optionally substituted with one or more groups selected from the group consisting of halo, aryl, amino, heteroalkyl, heteroalkenyl, heterocycloalkyl, and heteroaryl;
R₈ₐ and R_{8b} are each independently selected from the group consisting of H and optionally substituted C₁₋₁₀ alkyl;
R₉ₐ is selected from the group consisting of H, optionally substituted C₁₋₁₀ alkyl, C(═O)C₁₋₆ alkyl, C₁₋₆ alkylene-OH, C₁₋₆ alkylene-O—C₁₋₆ alkyl, C₁₋₆ alkylene-cyclolkyl, C(═O)C₁₋₆ alkylene-cycloalkyl, C(═O)cycloalkyl, C(═O)heterocycloalkyl, C(═O)aryl, C(═O)heteroaryl, or C(═O)NH-aryl;
one of R₁₀ₐ and R_{10b} is selected from the group consisting of H and optionally substituted C₁₋₁₀ alkyl, and the other of R₁₀ₐ and R_{10b} is
   Lₐ-L_{b}-L_{c}-L_{d}, wherein:
   Lₐ is C₂₋₆ alkenylene or C₁₋₆ alkylene, wherein one carbon atom of C₁₋₆ alkylene may be replaced by oxo

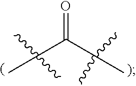 [SMILES: *C(*)=O]

L_{b} is absent, or is optionally substituted cycloalkyl or optionally substituted heterocycloalkyl;
L_{c} is absent or is C₁₋₆ alkylene, C₁₋₆ alkylene-N(C₁₋₆ alkyl), NH, N—C₁₋₆ alkyl, N—C₁₋₆ cycloalkyl, —NH—C₁₋₆ alkylene, —NH—C₁₋₆ alkylene-heteroarylene, —N(C₁₋₆ alkyl)-C₁₋₆ alkylene, —N—C₁₋₆ alkylene-cycloalkyl, N—C₁₋₆ alkylene-heterocycloalkyl, N—C₁₋₆ alkylene-aryl, N—C₁₋₆ alkylene-heteroaryl, C(═O), C(═O)O—, OC(O)—, C(═O)NH, C(═O)N-alkyl, C(═O)NH—C₁₋₆ alkylene, C(═O)N(C₁₋₆ alkyl)-C₁₋₆ alkylene, N(C₁₋₆ alkyl)-C(═O)—C₁₋₆ alkylene, SO₂, SO₂NH, SO₂N—C₁₋₆ alkyl, SO₂N—(C₁₋₆ alkyl)-(C₁₋₆ alkylene), OC(═O)—NH, OC(═O)—N-alkyl, SO₂, SO₂C₁₋₆ alkylene, SO₂NH, SO₂N(C₁₋₆ alkyl), SO₂NH—(C₁₋₆ alkylene, SO₂N(C₁₋₆ alkyl)-(C₁₋₆ alkylene), SO₂N(C₁₋₆ alkylene-aryl), SO₂N(C₁₋₆ alkylene-heteroaryl),

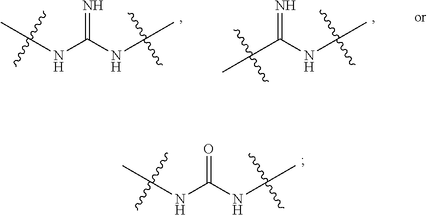 [SMILES: *NC(=N)N* ; *NC(*)=N ; *NC(=O)N*]

L_{d} is H, C₁₋₆ alkyl, OH, alkoxy, NH₂, NHC₁₋₆ alkyl, N(C₁₋₆ alkyl)₂, C₁₋₆ alkyl, C₁₋₆ haloalkyl, optionally substituted cycloalkyl, optionally substituted heterocycloalkyl, optionally substituted aryl, optionally substituted heteroaryl, optionally substituted benzyl; and
R₁₁ₐ and R_{11b} are each independently selected from the group consisting of —H and optionally substituted C₁₋₁₀ alkyl;
wherein “ [symbol] ” indicates a point of attachment.

Pharmaceutical compositions comprising the compounds are also described herein, as are processes for preparing the compounds.

### DETAILED DESCRIPTION

### Definitions

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety, including U.S. Pat. Publ. No. 2013/0090326. In case of conflict, the present specification, including these definitions, will control.

The terms “a,” “an,” and “the” as used herein not only include aspects with one member, but also include aspects with more than one member.

The term “about” as used herein means “approximately” and is used to modify a numerical value indicates a defined range around that value. If “X” were the value, “about X” would generally indicate a value from 0.95X to 1.05X. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.” When the quantity “X” only includes whole-integer values (e.g., “X carbons”), “about X” indicates from (X−1) to (X+1). In this case, “about X” as used herein specifically indicates at least the values X, X−1, and X+1.

When “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 7, 9, or 11%” is equivalent to “about 7%, about 9%, or about 11%.”

The following abbreviations and terms have the indicated meanings throughout:

| Abbreviation | Meaning |
|---|---|
| AcOH | acetic acid |
| APC | Adenomatous polyposis coli |
| br | broad |
| ° C. | degrees Celsius |
| conc | concentrated |
| CF | Cystic fibrosis |
| d | doublet |
| dd | doublet of doublet |
| dt | doublet of triplet |
| DCM | dichloromethane |
| DIEA or DIPEA | N,N-di-isopropyl-N-ethylamine |
| DMA | N,N-dimethylacetamide |
| DME | 1,2-dimethoxyethane |
| DMF | N,N-dimethylformamide |
| DMSO | dimethyl sulfoxide |
| dppf | 1,1′-bis(diphenylphosphano)ferrocene |
| EI | Electron Impact ionization |
| equiv | equivalents |
| FAP | Familial adenomatous polyposis |
| FFPE | Formalin fixed, paraffin embedded |
| g | gram(s) |
| GC/MS | gas chromatography/mass spectrometry |
| h or hr | hour(s) |
| HATU | 2-(1H-7-azabenzotriazol-1-yl)-1,1,3,3-tetramethyl |
| | uronium hexafluorophosphate |
| HPLC | high pressure liquid chromatography |
| JEB | Junctional epidermolysis bullosa |
| L | liter(s) |
| LC/MS | liquid chromatography/mass spectrometry |
| M | molar or molarity |
| m | Multiplet |
| MeOH | methanol |
| mg | milligram(s) |
| MHz | megahertz (frequency) |
| min | minute(s) |
| mL | milliliter(s) |
| μL | microliter(s) |
| μM | micromolar |
| μmol | micromole(s) |
| mM | Millimolar |
| mmol | millimole(s) |
| mol | mole(s) |
| MS | mass spectral analysis |
| Ms | mesyl |
| N | normal or normality |
| NBF | Neutral buffered formalin |
| nM | Nanomolar |
| NMR | nuclear magnetic resonance spectroscopy |
| q | Quartet |
| quant | quantitative |
| RDEB | Recessive dystrophic epidermolysis bullosa |
| rt | Room temperature |
| s | Singlet |
| t or tr | Triplet |
| THF | tetrahydrofuran |
| Ts | tosyl |

The symbol “ [symbol] ” means a single bond, “ [symbol] ” means a double bond, “ [symbol] ” means a triple bond, “ [symbol] ” means a single or double bond. The symbol “ [symbol] ” refers to a group on a double-bond as occupying either position on the terminus of a double bond to which the symbol is attached; that is, the geometry, E- or Z-, of the double bond is ambiguous. When a group is depicted removed from its parent Formula, the “ [symbol] ” symbol will be used at the end of the bond which was theoretically cleaved in order to separate the group from its parent structural Formula.

When chemical structures are depicted or described, unless explicitly stated otherwise, all carbons are assumed to have hydrogen substitution to conform to a valence of four. For example, in the structure on the left-hand side of the schematic below there are nine hydrogens implied. The nine hydrogens are depicted in the right-hand structure. Sometimes a particular atom in a structure is described in textual Formula as having a hydrogen or hydrogens as substitution (expressly defined hydrogen), for example, —CH₂CH₂—. It is understood by one of ordinary skill in the art that the aforementioned descriptive techniques are common in the chemical arts to provide brevity and simplicity to description of otherwise complex structures.

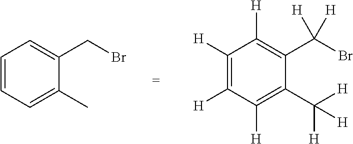

If a group “R” is depicted as “floating” on a ring system, as for example in the following Formula.

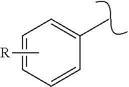

then, unless otherwise defined, a substituent “R” may reside on any atom of the ring system, assuming replacement of a depicted, implied, or expressly defined hydrogen from one of the ring atoms, so long as a stable structure is formed.

If a group “R” is depicted as floating on a fused or bridged ring system, as for example in the following Formulas.

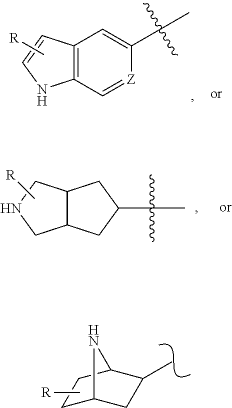

then, unless otherwise defined, a substituent “R” may reside on any atom of the fused or bridged ring system, assuming replacement of a depicted hydrogen (for example the —NH— in the Formula above), implied hydrogen (for example as in the Formula above, where the hydrogens are not shown but understood to be present), or expressly defined hydrogen (for example where in the Formula above, “Z” equals=CH—) from one of the ring atoms, so long as a stable structure is formed. In the example depicted, the “R” group may reside on either the 5-membered or the 6-membered ring of the fused or bridged ring system.

When a group “R” is depicted as existing on a ring system containing saturated carbons, as for example in the following Formula

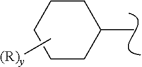

where, in this example, “y” can be more than one, assuming each replaces a currently depicted, implied, or expressly defined hydrogen on the ring; then, unless otherwise defined, where the resulting structure is stable, two “R's” may reside on the same carbon. In another example, two R's on the same carbon, including that carbon, may form a ring, thus creating a spirocyclic ring structure with the depicted ring as for example in the following Formula

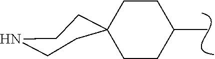

The term “acyl” as used herein includes an alkanoyl, aroyl, heterocycloyl, or heteroaroyl group as defined herein. Examples of acyl groups include, but are not limited to, acetyl, benzoyl, and nicotinoyl.

The term “alkanoyl” as used herein includes an alkyl-C(O)— group wherein the alkyl group is as defined herein. Examples of alkanoyl groups include, but are not limited to, acetyl and propanoyl.

The term “agent” as used herein includes a compound or mixture of compounds that, when added to a composition, tend to produce a particular effect on the composition's properties.

For example, a composition comprising a thickening agent is likely to be more viscous than an otherwise identical comparative composition that lacks the thickening agent.

The term “alkenyl” as used herein includes a straight or branched chain hydrocarbon containing at least one carbon-carbon double bond. The chain may contain an indicated number of carbon atoms. For example, “C₁-C₁₂ alkenyl” indicates that the group may have from 1 to 12 (inclusive) carbon atoms and at least one carbon-carbon double bond. When the indicated number of carbon atoms is 1, then the Cᵢ alkenyl is double bonded to a carbon (i.e., a carbon equivalent to an oxo group). In certain aspects, the chain includes 1 to 12, about 2 to 15, about 2 to 12, about 2 to 8, or about 2 to 6 carbon atoms. An alkenyl group can be preferably one stereoisomer (i.e., cis- or, alternatively, trans-). Examples of an alkenyl group may include, but are not limited to, ethenyl (i.e., vinyl), allyl, propenyl, butenyl, crotyl, pentenyl, hexenyl, heptenyl, octenyl, nonenyl, decenyl, dodecenyl, cyclopentenyl, cyclohexenyl, 2-isopentenyl, allenyl, butadienyl, pentadienyl, 3-(1,4-pentadienyl), and hexadienyl.

An alkenyl group can be unsubstituted or optionally substituted. When optionally substituted, one or more hydrogen atoms of the alkenyl group (e.g., from 1 to 4, from 1 to 2, or 1) may be replaced with a moiety independently selected from the group consisting of fluoro, hydroxy, alkoxy, amino, alkylamino, acylamino, thio, and alkylthio, with the proviso that no hydrogen atom substituent on the carbon-carbon double bond is replaced by a hydroxy, amino, or thio group. In some aspects, the alkenyl group is unsubstituted or not optionally substituted.

“Alkenylene” as used herein includes an alkenyl group that is substituted at two points. An example is but-2-enylene (—CH₂CH═CHCH₂—) and the like.

The term “alkyl” as used herein includes an aliphatic hydrocarbon chain that may be straight chain or branched. The chain may contain an indicated number of carbon atoms: For example, C₁-C₁₀ indicates that the group may have from 1 to 10 (inclusive) carbon atoms in it. If not otherwise indicated, an alkyl group contains from 1 to about 20 carbon atoms. In some aspects, alkyl groups have 1 to about 10 carbon atoms. In some aspects, alkyl groups (“lower alkyl”) have 1 to 8, 1 to 6, or 1 to 3 carbon atoms in the chain. Examples may include, but are not limited to, methyl, ethyl, propyl, isopropyl (iPr), 1-butyl, 2-butyl, isobutyl (iBu), tert-butyl, pentyl, 2-methylbutyl, 1,1-dimethylpropyl, hexyl, heptyl, octyl, nonyl, decyl, docecyl, cyclopentyl, or cyclohexyl.

An alkyl group can be unsubstituted or optionally substituted. When optionally substituted, one or more hydrogen atoms of the alkyl group (e.g., from 1 to 4, from 1 to 2, or 1) may be replaced with a moiety independently selected from the group consisting of chloro, fluoro, hydroxy, alkoxy, amino, alkylamino, acylamino, thio, and alkylthio. In some aspects, the alkyl group is unsubstituted or not optionally substituted.

“Alkylene” as used herein includes an alkyl group that is substituted at two points. An example is methylene (—CH₂—), propylene (—CH₂CH₂CH₂—), and the like.

The term “alkoxy” as used herein includes a straight or branched chain saturated or unsaturated hydrocarbon containing at least one oxygen atom in an ether group (e.g., EtO—). The chain may contain an indicated number of carbon atoms. For example, “C₁-C₁₂ alkoxy” indicates that the group may have from 1 to 12 (inclusive) carbon atoms and at least one oxygen atom. Examples of a C₁-C₁₂ alkoxy group include, but are not limited to, methoxy, ethoxy, isopropoxy, butoxy, n-pentoxy, isopentoxy, neopentoxy, and hexoxy.

An alkoxy group can be unsubstituted or optionally substituted. When optionally substituted, one or more hydrogen atoms of the alkoxy group (e.g., from 1 to 4, from 1 to 2, or 1) may be replaced with a moiety independently selected from the group consisting of fluoro, hydroxy, alkoxy, amino, alkylamino, acylamino, thio, and alkylthio, with the proviso that no hydrogen atom alpha to the ether oxygen is replaced by a hydroxy, amino, or thio group. In some aspects, the alkoxy group is unsubstituted or not optionally substituted.

The term “alkynyl” as used herein includes a straight, branched, or cyclic hydrocarbon containing at least one carbon-carbon triple bond. Examples may include, but are not limited to, ethynyl, propargyl, propynyl, butynyl, pentynyl, hexynyl, heptynyl, octynyl, nonynyl, decynyl, or decynyl.

“Alkynylene” as used herein includes an alkynyl group that is substituted at two points. An example is 2-butynylene (—CH₂CCCH₂—) and the like.

An alkynyl group can be unsubstituted or optionally substituted. When optionally substituted, one or more hydrogen atoms of the alkynyl group (e.g., from 1 to 4, from 1 to 2, or 1) may be replaced with a moiety independently selected from the group consisting of fluoro, hydroxy, alkoxy, amino, alkylamino, acylamino, thio, and alkylthio, with the proviso that no sp-hybridized hydrogen atom substituent is replaced by a hydroxy, amino, or thio group. In some aspects, the alkynyl group is unsubstituted or not optionally substituted.

The term “aryl” as used herein includes cyclic aromatic carbon ring systems containing from 6 to 18 carbons. Examples of an aryl group include, but are not limited to, phenyl, naphthyl, anthracenyl, tetracenyl, biphenyl and phenanthrenyl.

An aryl group can be unsubstituted or optionally substituted. When optionally substituted, one or more hydrogen atoms of the aryl group (e.g., from 1 to 5, from 1 to 2, or 1) may be replaced with a moiety independently selected from the group consisting of alkyl, cyano, acyl, halo, haloalkyl, hydroxy, alkoxy, amino, alkylamino, acylamino, thio, and alkylthio. In some aspects, the alkoxy group is unsubstituted or not optionally substituted.

The term “arylalkyl” or “aralkyl” as used herein includes an alkyl group as defined herein where at least one hydrogen substituent has been replaced with an aryl group as defined herein. Examples include, but are not limited to, benzyl, 1-phenylethyl, 4-methylbenzyl, and 1,1,-dimethyl-1-phenylmethyl.

An arylalkyl or aralkyl group can be unsubstituted or optionally substituted as per its component groups. For example, but without limitation, the aryl group of an arylalkyl group can be substituted, such as in 4-methylbenzyl. In some aspects, the group is unsubstituted or not optionally substituted, especially if including a defined substituent, such as a hydroxyalkyl or alkylaminoalkoxy group.

The term “cycloalkyl” as used herein includes non-aromatic saturated monocyclic or multicyclic ring system that may contain an indicated number of carbon atoms. For example, C₃-C₁₂ indicates that the group may have from 3 to 12 (inclusive) carbon atoms in it. If not otherwise indicated, a cycloalkyl group includes about 3 to about 20 carbon atoms. In some aspects, cyclo alkyl groups have 3 to about 12 carbon atoms in the group. In some aspects, cycloalkyl groups have 3 to about 7 carbon atoms in the group. Examples may include, but are not limited to, cyclopropyl, cyclobutyl, cyclopentyl, cyclohexyl, 4,4-dimethylcyclohexyl, and cycloheptyl.

A cycloalkyl group can be unsubstituted or optionally substituted. When optionally substituted, one or more hydrogen atoms of the cycloalkyl group (e.g., from 1 to 4, from 1 to 2, or 1) may be replaced with a moiety independently selected from the group consisting of alkyl, halo, haloalkyl, hydroxy, alkoxy, oxo, amino, alkylamino, acylamino, thio, and alkylthio. In some aspects, a substituted cycloalkyl group can incorporate an exo- or endocyclic alkene (e.g., cyclohex-2-en-1-yl). In some aspects, a cycloalkyl group is unsubstituted or not optionally substituted.

As used herein, “fluoroalkyl” includes an alkyl group wherein the alkyl group includes one or more fluoro-substituents. Examples include, but are not limited to, trifluoromethyl.

As used herein, “geminal” substitution includes two or more substituents that are directly attached to the same atom. An example is 3,3-dimethyl substitution on a cyclohexyl or spirocyclohexyl ring.

As used herein, “halo” or “halogen” includes fluoro, chloro, bromo, and iodo.

The term “heteroaryl” or “heterocycloaryl” includes mono and bicyclic groups that are completely unsaturated or partically unsaturated of about 4 to about 14 ring atoms (e.g., 4 to 10 or 5 to 10 atoms) containing at least one heteroatom. Heteroatom as used in the term heteroaryl refers to oxygen, sulfur and nitrogen. A nitrogen atom of a heteroaryl is optionally oxidized to the corresponding N-oxide. Examples include, but are not limited to, pyrazinyl, furanyl, thienyl, pyridyl, pyrimidinyl, isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, imidazo[1,2-a]pyridine, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, and benzothiazolyl. Other examples include

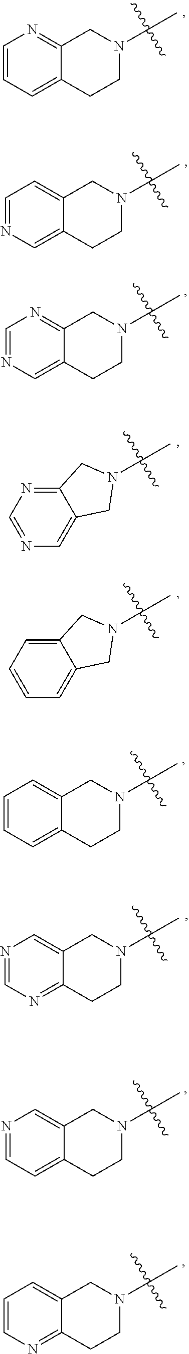

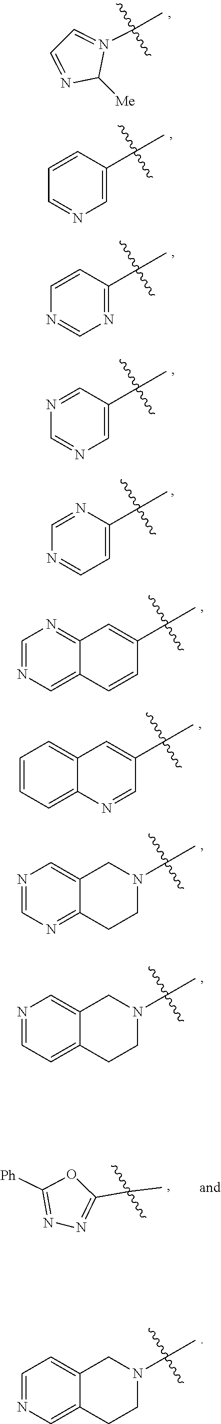

The term “heteroarylene” or “heterocycloarylene” as used herein includes a heteroaryl group that is substituted at two points.

An heteroaryl group can be unsubstituted or optionally substituted. When optionally substituted, one or more hydrogen atoms of the heteroaryl group (e.g., from 1 to 5, from 1 to 2, or 1) may be replaced with a moiety independently selected from the group consisting of alkyl, cyano, acyl, halo, haloalkyl, hydroxy, oxo, alkoxy, amino, alkylamino, acylamino, thio, and alkylthio. In some aspects, the heteroaryl group is unsubstituted or not optionally substituted.

The term “heteroaroyl” as used herein includes a heteroaryl-C(O)— group wherein heteroaryl is as defined herein. Heteroaroyl groups include, but are not limited to, thiophenoyl, nicotinoyl, pyrrol-2-ylcarbonyl, and pyridinoyl.

The term “heterocycloalkyl” may be used interchangeably herein, and as used herein includes a heterocyclyl-C(O)— group wherein heterocyclyl is as defined herein. Examples include, but are not limited to, N-methyl prolinoyl and tetrahydrofuranoyl.

As used herein, “heterocyclyl” (heterocyclo; heterocyclic; heterocycloalkyl) includes a non-aromatic saturated ring of about 3 to about 8 ring atoms (e.g., 5 to about 10 ring atoms, or 3 to about 6 ring atoms), in which one or more of the atoms in the ring system is an element or elements other than carbon, e.g., nitrogen, oxygen or sulfur. A heterocyclyl group optionally comprises at least one sp²-hybridized atom (e.g., a ring incorporating an carbonyl, endocyclic olefin, or exocyclic olefin). In some embodiments, a nitrogen or sulfur atom of the heterocyclyl is optionally oxidized to the corresponding N-oxide, S-oxide or S,S-dioxide. The monocyclic heterocycle means a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyridazin-3(2H)-onyl, pyridin-2(1H)-onyl, pyrrolinyl, pyrrolidinyl, tetrahydrofuranyl, tetrahydropyranyl, tetrahydropyridinyl, tetrahydropyrimidinyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl.

The term “heterocycloalkylene” as used herein includes a heterocyclyl (heterocyclo; heterocyclic) group that is substituted at two points.

The term “heterocyclyl” also includes multicyclic rings such as a bicyclic heterocycle, or a tricyclic heterocycle which may be in a fused, bridged, or spiro orientation. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. Representative examples of bicyclic heterocycles include, but are not limited to, 3-azabicyclo[3.1.0]hexane, 3-azabicyclo[4.1.0]heptane, 3-azabicyclo[3.2.0]heptane, (3aR,6aS)-hexahydro-1H-2λ₂-cyclopenta[c]pyrrole, (3aR,7aS)-octahydro-2λ₂-isoindole.

Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms.

A heterocycyl group can be unsubstituted or optionally substituted. When optionally substituted, one or more hydrogen atoms of the group (e.g., from 1 to 4, from 1 to 2, or 1) may be replaced with a moiety independently selected from the group consisting of alkyl, halo, haloalkyl, oxo, acetyl, hydroxy, alkoxy, amino, alkylamino, acylamino, thio, and alkylthio. In some aspects, a substituted heterocycyl group can incorporate an exo- or endocyclic alkene (e.g., cyclohex-2-en-1-yl). In some aspects, the heterocycyl group is unsubstituted or not optionally substituted.

The monocyclic, bicyclic, and tricyclic heterocycles are connected to the parent molecular moiety through any carbon atom or any nitrogen atom contained within the rings, and can be unsubstituted or substituted.

As used herein, the term “hydrophilic moiety” or “hydrophilic group” includes a moiety or a functional group that has a strong affinity to water. Examples may include, but are not limited to, a charged moiety, such as a cationic moiety or an anionic moiety, or a polar uncharged moiety, such as an alkoxy group or an amine group.

As used herein, the term “hydroxyalkyl” includes an alkyl group where at least one hydrogen substituent has been replaced with an alcohol (—OH) group. In certain aspects, the hydroxyalkyl group has one alcohol group. In certain aspects, the hydroxyalkyl group has one or two alcohol groups, each on a different carbon atom. In certain aspects, the hydroxyalkyl group has 1, 2, 3, 4, 5, or 6 alcohol groups. Examples may include, but are not limited to, hydroxymethyl, 2-hydroxyethyl, and 1-hydroxyethyl.

When any two substituent groups or any two instances of the same substituent group are “independently selected” from a list of alternatives, the groups may be the same or different. For example, if R^{a} and R^{b} are independently selected from the group consisting of alkyl, fluoro, amino, and hydroxyalkyl, then a molecule with two R^{a} groups and two R^{b} groups could have all groups be an alkyl group (e.g., four different alkyl groups). Alternatively, the first R^{a} could be alkyl, the second R^{a} could be fluoro, the first R^{b} could be hydroxyalkyl, and the second R^{b} could be amino (or any other substituents taken from the group). Alternatively, both R^{a} and the first R^{b} could be fluoro, while the second R^{b} could be alkyl (i.e., some pairs of substituent groups may be the same, while other pairs may be different).

“Amino protecting group” is a protecting group that is suitable for preventing undesired reactions at an amino nitrogen. Representative amino-protecting groups include, but are not limited to, formyl; acyl groups, for example alkanoyl groups, such as acetyl; alkoxycarbonyl groups, such as tert-butoxycarbonyl (Boc); arylmethoxycarbonyl groups, such as benzyloxycarbonyl (Cbz) and 9-fluorenylmethoxycarbonyl (Fmoc); arylmethyl groups, such as benzyl (Bn), trityl (Tr), and 1,1-di-(4′-methoxyphenyl)methyl; silyl groups, such as trimethylsilyl (TMS) and tert-butyldimethylsilyl (TBDMS); and the like.

“Hydroxyl protecting group” is a protecting group that is suitable for preventing undesired reactions at a hydroxyl oxygen. Representative hydroxyl protecting groups include, but are not limited to, acyl groups such as acetyl; arylmethyl groups, such as benzyl (Bn), trityl (Tr), and 1,1-di-(4′-methoxyphenyl)methyl; silyl groups, such as trimethylsilyl (TMS) and tert-butyldimethylsilyl (TBDMS); ethers such as methoxymethyl (MOM), tetrahydropyranyl (THP), and benzyl (Bn); and the like.

“Yield” for each of the reactions described herein is expressed as a percentage of the theoretical yield.

“Subject and “patient” are used interchangeably. A “subject” or “patient” for the purposes of the present invention includes humans and other animals, particularly mammals, and other organisms. Thus the methods are applicable to both human therapy and veterinary applications. In a specific embodiment the patient is a mammal, and in a more specific embodiment the patient is human.

A “pharmaceutically acceptable salt” of a compound means a salt that is pharmaceutically acceptable and that possesses the desired pharmacological activity of the parent compound. It is understood that the pharmaceutically acceptable salts are non-toxic. Additional information on suitable pharmaceutically acceptable salts can be found in Remington's Pharmaceutical Sciences, 17^{th} ed., Mack Publishing Company, Easton, P A, 1985, which is incorporated herein by reference or S. M. Berge, et al., “Pharmaceutical Salts,” J. Pharm. Sci., 1977; 66:1-19 both of which are incorporated herein by reference.

Examples of pharmaceutically acceptable acid addition salts include those formed with inorganic acids such as hydrochloric acid, hydrobromic acid, sulfuric acid, nitric acid, phosphoric acid, and the like; as well as organic acids such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, and salicylic acid and the like.

Examples of a pharmaceutically acceptable base addition salts include those formed when an acidic proton present in the parent compound is replaced by a metal ion, such as sodium, potassium, lithium, ammonium, calcium, magnesium, iron, zinc, copper, manganese, aluminum salts and the like. Specific salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins. Examples of organic bases include isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, tromethamine, N-methylglucamine, polyamine resins, and the like. Exemplary organic bases are isopropylamine, diethylamine, ethanolamine, trimethylamine, dicyclohexylamine, choline, and caffeine.

“Therapeutically effective amount” is an amount of a compound of the invention, that when administered to a patient, ameliorates a symptom of the disease. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art having regard to their knowledge and to this disclosure.

The phrase “genetic disease”, as used herein, means a genetic disorder, genetic disease, genetic condition or genetic syndrome.

“Preventing” or “prevention” of a disease, disorder, or syndrome includes inhibiting the disease from occurring in a human, i.e. causing the clinical symptoms of the disease, disorder, or syndrome not to develop in an animal that may be exposed to or predisposed to the disease, disorder, or syndrome but does not yet experience or display symptoms of the disease, disorder, or syndrome.

“Treating” or “treatment” of a disease, disorder, or syndrome, as used herein, includes (i) inhibiting the disease, disorder, or syndrome, i.e., arresting its development; and (ii) relieving the disease, disorder, or syndrome, i.e., causing regression of the disease, disorder, or syndrome. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

CFTR modulators are drugs or compounds that target the underlying defect in the cystic fibrosis transmembrance conductance regulator (CFTR) protein. Two main types of modulators are potentiators and correctors. [accessed on May 24, 2022, Cystic Fibrosis Foundation https://www.cff.org/Research/Developing-New-Treatments/CFTR-Modulator-Types/].

### EMBODIMENTS

In an aspect, what is provded is a method for treating a subject having a genetic disease comprising:
administering a therapeutically effective amount of a compound of Formula I

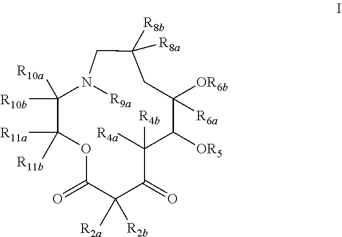

or a pharmaceutically acceptable salt thereof, wherein:
one of R₂ₐ and R_{2b} is selected from the group consisting of H, halo, optionally substituted C₁₋₁₀ alkyl, optionally substituted C₁₋₁₀ alkoxy, and optionally substituted C₂₋₁₀ alkenyl, wherein C₁₋₁₀ alkyl, C₁₋₁₀ alkoxy, and C₂₋₁₀ alkenyl are optionally substituted with one or more groups selected from the group consisting of halo, aryl, amino, alkyl, heteroalkyl, heteroalkenyl, heterocycloalkyl, and heteroaryl; and the other of R₂ₐ and R_{2b} is selected from the group consisting of halo, optionally substituted C₁₋₁₀ alkyl, optionally substituted C₁₋₁₀ alkoxy, and optionally substituted C₂₋₁₀ alkenyl, wherein C₁₋₁₀ alkyl, C₁₋₁₀ alkoxy, and C₂₋₁₀ alkenyl are optionally substituted with one or more groups selected from the group consisting of halo, aryl, amino, alkyl, heteroalkyl, heteroalkenyl, heterocycloalkyl, and heteroaryl;
each of R₄ₐ and R_{4b} is independently selected from the group consisting of H and optionally substituted C₁₋₁₀ alkyl;
R₅ is selected from the group consisting of H, a hydroxyl protecting group, and

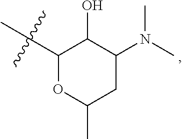 [SMILES: *C1OC(C)CC(N(C)C)C1O]

wherein “ [symbol] ” indicates appoint of attachment;
R₆ₐ is optionally substituted C₁₋₁₀ alkyl;
R_{6b} is H, C₁₋₁₀ alkyl, C₁₋₁₀ hydroxyalkyl, allyl, haloalkyl, aryl, heteroalkenyl, heterocycloalkyl, or heteroaryl, any of which can be optionally substituted with one or more groups selected from the group consisting of halo, aryl, amino, heteroalkyl, heteroalkenyl, heterocycloalkyl, and heteroaryl;
R₈ₐ and R_{8b} are each independently selected from the group consisting of H and optionally substituted C₁₋₁₀ alkyl;
R₉ₐ is selected from the group consisting of H, optionally substituted C₁₋₁₀ alkyl, C(═O)C₁₋₆ alkyl, C₁₋₆ alkylene-OH, C₁₋₆ alkylene-O—C₁₋₆ alkyl, C₁₋₆ alkylene-cyclolkyl, C(═O)C₁₋₆ alkylene-cycloalkyl, C(═O)cycloalkyl, C(═O)heterocycloalkyl, C(═O)aryl, C(═O)heteroaryl, or C(═O)NH-aryl;
one of R₁₀ₐ and R_{10b} is selected from the group consisting of H and optionally substituted C₁₋₁₀ alkyl, and the other of R₁₀ₐ and R_{10b} is
   Lₐ-L_{b}-L_{c}-L_{d} wherein:
   Lₐ is C₂₋₆ alkyenylene or C₁₋₆ alkylene, wherein one carbon atom of C₁₋₆ alkylene may be replaced by oxo

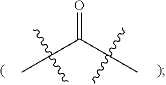 [SMILES: *C(*)=O]

L_{b} is absent, or is optionally substituted cycloalkyl or optionally substituted heterocycloalkyl;
L_{c} is absent or is C₁₋₆ alkylene, C₁₋₆ alkylene-N(C₁₋₆ alkyl), NH, N—C₁₋₆ alkyl, N—C₁₋₆ cycloalkyl, —NH—C₁₋₆ alkylene, —NH—C₁₋₆ alkylene-heteroarylene, —N(C₁₋₆ alkyl)-C₁₋₆ alkylene, —N—C₁₋₆ alkylene-cycloalkyl, N—C₁₋₆ alkylene-heterocycloalkyl, N—C₁₋₆ alkylene-aryl, N—C₁₋₆ alkylene-heteroaryl, C(═O), C(═O)O—, OC(═O)—, C(═O)NH, C(═O)N-alkyl, C(═O)NH—C₁₋₆ alkylene, C(═O)N(C₁₋₆ alkyl)-C₁₋₆ alkylene, N(C₁₋₆ alkyl)-C(═O)—C₁₋₆ alkylene, SO₂, SO₂NH, SO₂N—C₁₋₆ alkyl, SO₂N—(C₁₋₆ alkyl)-(C₁₋₆ alkylene), OC(═O)—NH, OC(═O)—N-alkyl, SO₂, SO₂C₁₋₆ alkylene, SO₂NH, SO₂N(C₁₋₆ alkyl), SO₂NH—(C₁₋₆ alkylene), SO₂N(C₁₋₆ alkyl)-(C₁₋₆ alkylene), SO₂N(C₁₋₆ alkylene-aryl), SO₂N(C₁₋₆ alkylene-heteroaryl),

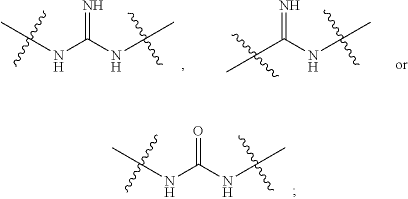 [SMILES: *NC(=N)N* ; *NC(*)=N ; *NC(=O)N*]

L_{d} is H, C₁₋₆ alkyl, OH, alkoxy, NH₂, NHC₁₋₆ alkyl, N(C₁₋₆ alkyl)₂, C₁₋₆ alkyl, C₁₋₆ haloalkyl, optionally substituted cycloalkyl, optionally substituted heterocycloalkyl optionally substituted aryl, optionally substituted heteroaryl, optionally substituted benzyl; and
R₁₁ₐ and R_{11b} are each independently selected from the group consisting of —H and optionally substituted C₁₋₁₀ alkyl;
wherein “ [symbol] ” indicates a point of attachment.

In an embodiment of the method, the compound is a compound of formula IA or a pharmaceutically acceptable salt thereof:

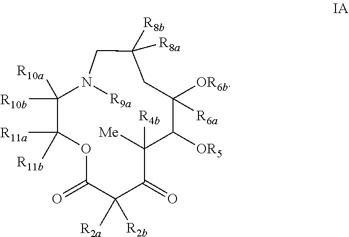

In another embodiment of the method, the compound is a compound of formula IB or a pharmaceutically acceptable salt thereof:

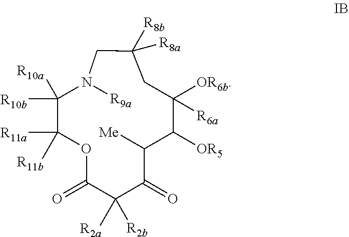

In another embodiment of the method, the compound is a compound of formula IC or a pharmaceutically acceptable salt thereof:

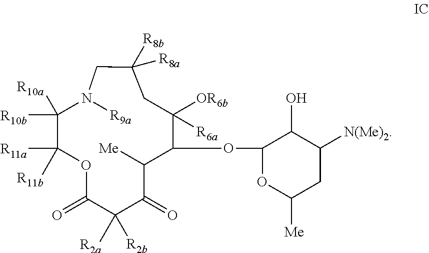

In another embodiment of the method, the compound is a compound of formula ID or a pharmaceutically acceptable salt thereof:

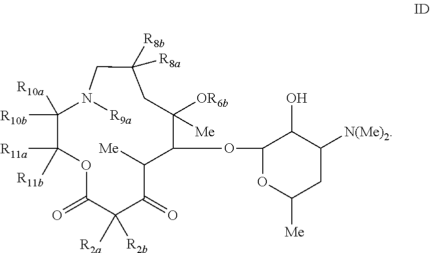

In this and other embodiments of the method, R_{6b} in the compound of formula 6D is selected from the group consisting of —H, C₁-C₁₀optionally substituted alkyl, optionally substituted C₁-C₁₀ hydroxyalkyl, and optionally substituted allyl. In this and other embodiments of the method, R_{6b} in the compound of formula 6D is selected from the group consisting of methyl, hydroxymethyl, hydroxyethyl, hydroxypropyl, hydroxybutyl, hydroxypentyl, hydroxyhexyl, —CH₂CHOHCH₂OH, and allyl.

In another embodiment of the method of the method, the compound is a compound of formula IE or a pharmaceutically acceptable salt thereof:

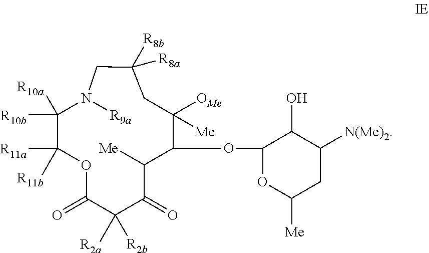

In another embodiment of the method of the method, the compound is a compound of formula IF or a pharmaceutically acceptable salt thereof:

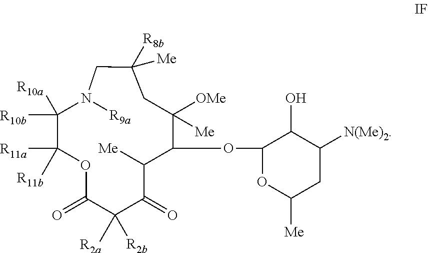

In another embodiment of the method of the method, the compound is a compound of formula IG or a pharmaceutically acceptable salt thereof:

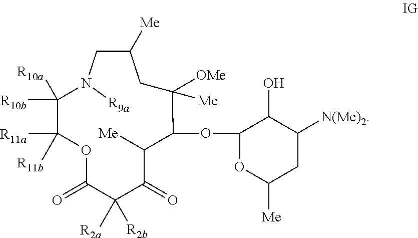

In this and other embodiments of the method, R₉ₐ in the recited compounds is —H, C₁₋₄ alkyl, or C₁₋₄ alkylene-OH.

In this and other embodiments of the method, wherein R₁₁ₐ and R_{11b} in the recited compounds are —H. In this and other embodiments of the method, one of R₁₁ₐ and R_{11b} in the recited compounds is —H and the other is optionally substituted C₁₋₁₀ alkyl. In this and other embodiments of the method, one of R₁₁ₐ and R_{11b} in the recited compounds is —H and the other is methyl. In this and other embodiments of the method, R₁₁ₐ and R_{11b} in the recited compounds are each independently optionally substituted C₁₋₁₀ alkyl. In this and other embodiments of the method, R₁₁ₐ and R_{11b} in the recited compounds are each methyl.

In this and other embodiments of the method, one of R₂ₐ and R_{2b} in the recited compounds is optionally substituted C₁₋₁₀ alkyl. In this and other embodiments of the method, one of R₂ₐ and R_{2b} in the recited compounds is methyl and the other of R₂ₐ and R_{2b} is H, or both of R₂ₐ and R_{2b} are methyl. In this and other embodiments of the method, one of R₂ₐ and R_{2b} in the recited compounds is methyl and the other is halo and more particularly fluoro or chloro.

In this and other embodiments of the method, one of R₂ₐ and R_{2b} in the recited compounds is methyl and the other is optionally substituted C₁₋₁₀ alkyl. In this and other embodiments of the method, one of R₂ₐ and R_{2b} in the recited compounds is methyl and the other is selected from the group consisting of optionally substituted C₁₋₁₀ alkyl, optionally substituted C₁₋₁₀ alkoxy, and optionally substituted C₁₋₁₀ alkenyl, wherein optionally substituted C₁₋₁₀ alkyl, optionally substituted C₁₋₁₀ alkoxy, and optionally substituted C₁₋₁₀ alkenyl are optionally substituted with one or more selected from the group consisting of halo, aryl, and heteroaryl.

In another embodiment, the compound is a compound of formula IH or a pharmaceutically acceptable salt thereof:

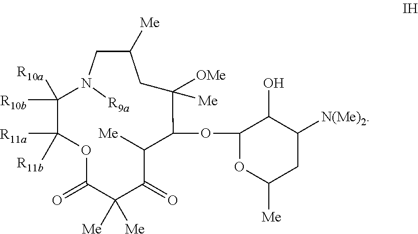

In this and other embodiments of the method, R₉ₐ in compounds of formula I and thus of formula IA-IH is —H or C₁₋₄ alkyl and one of R₁₀ₐ and R_{10b} in the recited compounds is —H or optionally substituted C₁₋₁₀ alkyl.

In this and other embodiments of the method, R₉ₐ in compounds of formula I and thus of formula IA-IH is —H.

In this and other embodiments of the method, R₉ₐ in compounds of formula I and thus of formula IA-IH is optionally substituted C₁₋₁₀ alkyl.

In this and other embodiments of the method, R₉ₐ in compounds of formula I and thus of formula IA-IH is methyl.

In another embodiment, the compound is a compound of formula IIA, IIB, IIC, or IID or a pharmaceutically acceptable salt thereof:

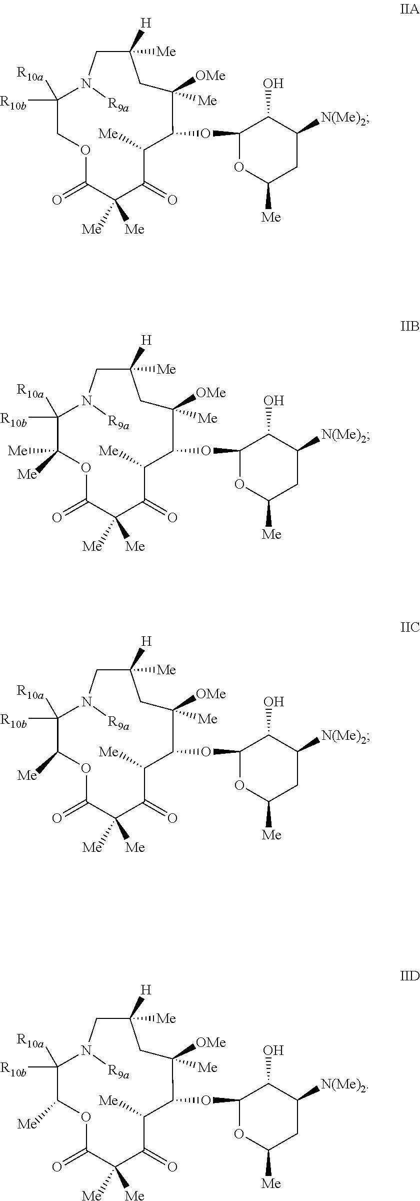

In another embodiment, the compound is a compound of formula IIA-1, IIA-2, IIB-1, IIB-2, IIC-1, IIC-2, IID-1, or IID-2 or a pharmaceutically acceptable salt thereof:

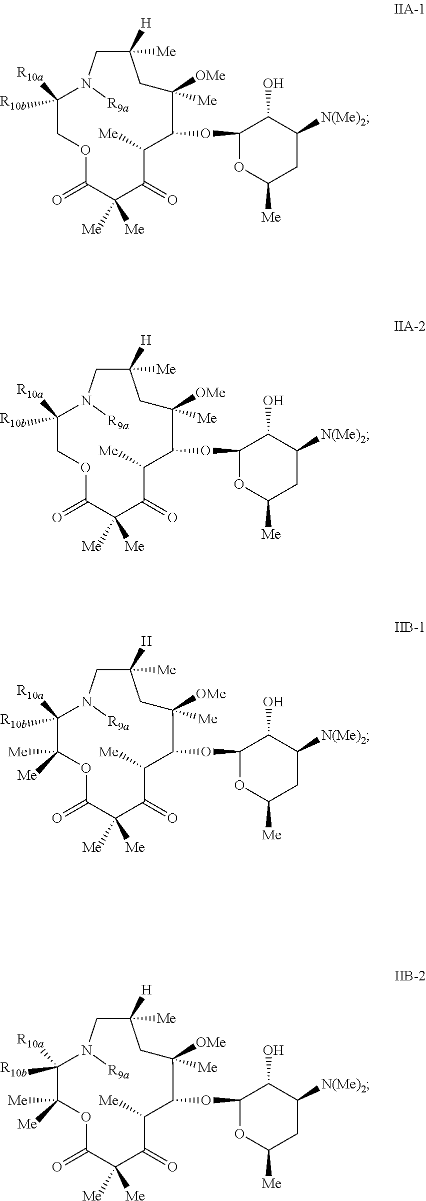

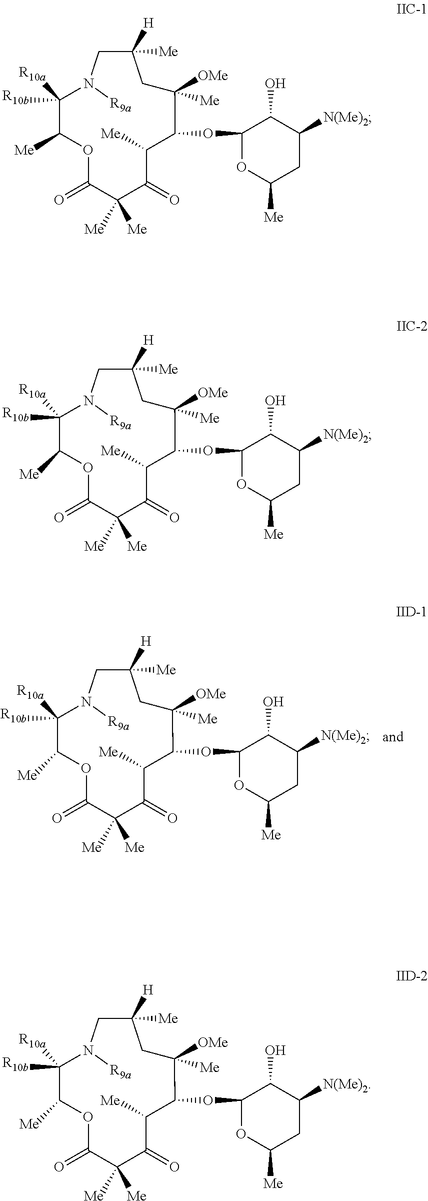

In another embodiment, the compound is a compound is a compound of formula IIA-1a, IIA-2a, IIB-1a, IIB-2a, IIC-1a, IIC-2a, IID-1a, or IID-2a or a pharmaceutically acceptable salt thereof:

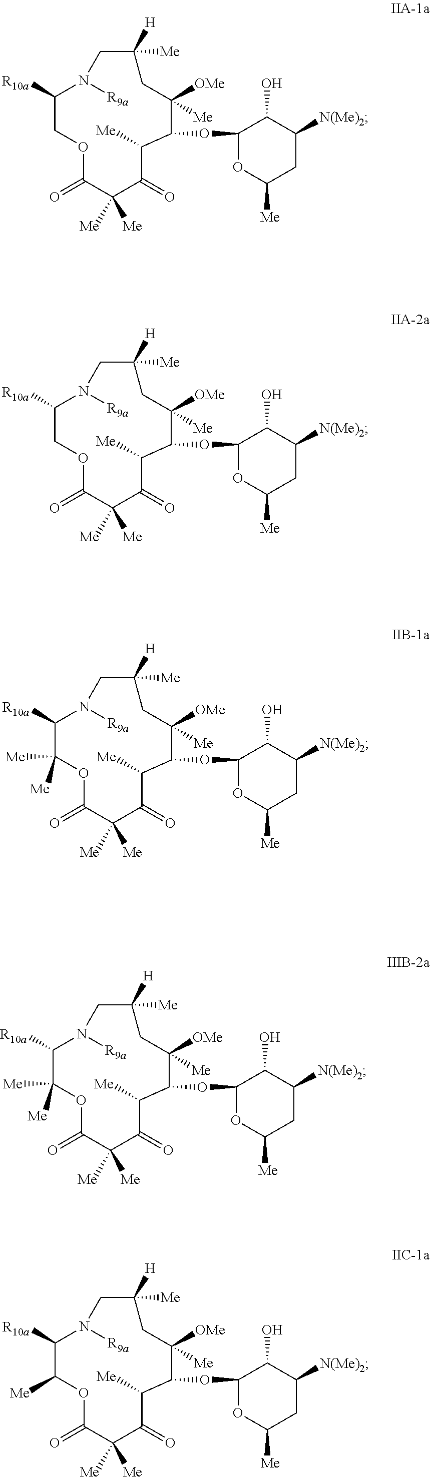

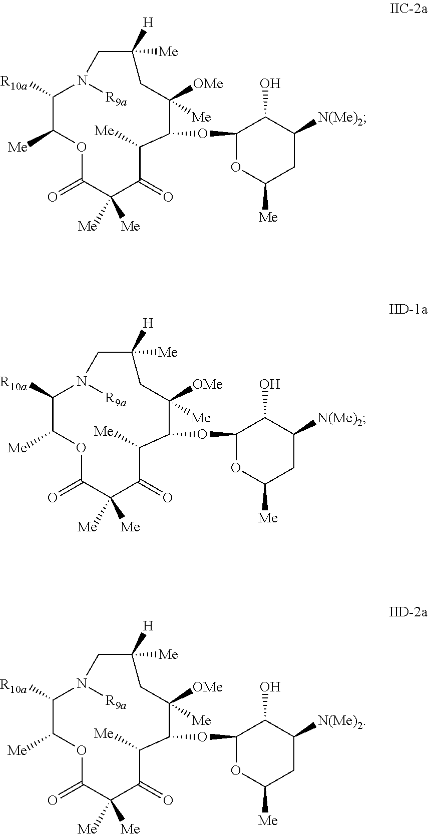

In another embodiment, the compound is a compound of formula IIA-1b, IIA-2b, IIB-1b, IIB-2b, IIC-1b, IIC-2b, IID-1b, or IID-2b or a pharmaceutically acceptable salt thereof:

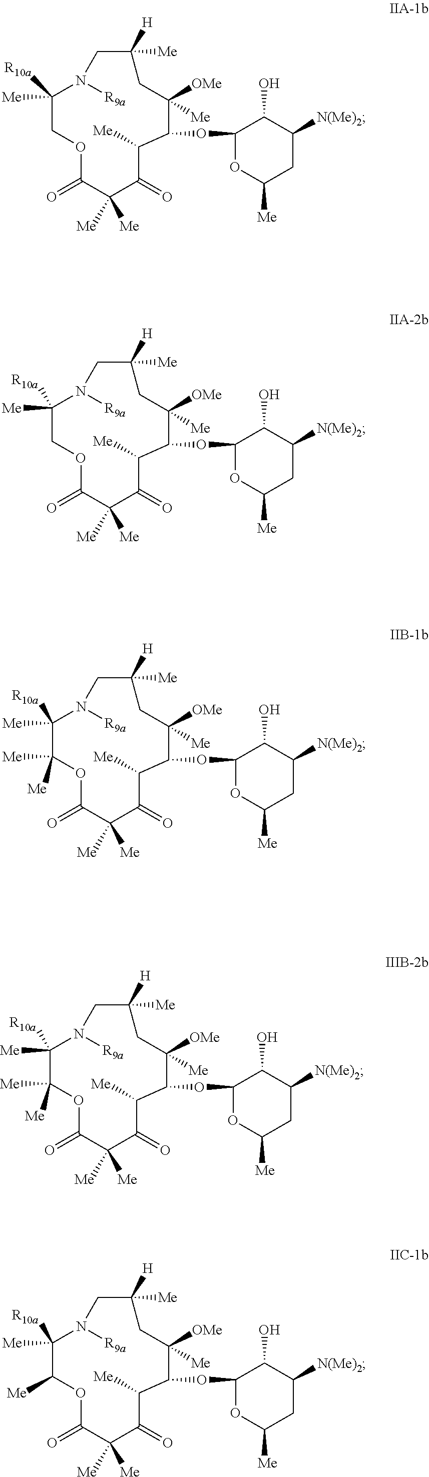

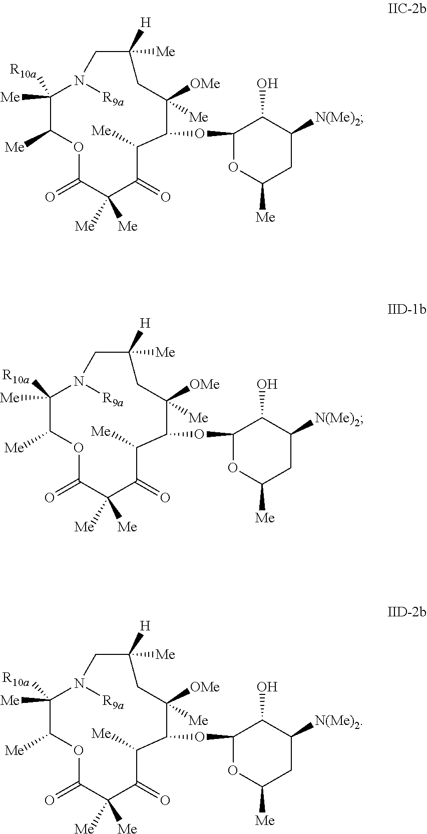

In the formula I and II compound for use in the method, R₉ₐ is —H, methyl, ethyl, propyl, isopropyl, butyl, isobutyl, pentyl, isopentyl, acetyl, C(═O)—NH-phenyl, C(═O)-ethyl, C(═O)-cyclopropyl, CH₂-cyclobutyl, CH₂CH₂CH₂OH, CH₂CH₂CH₂OMe, or CH₂CH₂OH. In some embodiments, R₉ₐ is —H, methyl, ethyl, propyl, isobutyl, isopentyl, acetyl, C(═O)—NH-phenyl, C(═O)-ethyl, C(═O)-cyclopropyl, CH₂-cyclobutyl, CH₂CH₂CH₂OMe, or CH₂CH₂OH. In some embodiments, R₉ₐ is —H, methyl, ethyl, propyl, isopropyl, butyl, isobutyl, pentyl, or isopentyl. In another embodiment, R₉ₐ is acetyl, C(═O)-phenyl, C(═O)-ethyl, C(═O)-cyclopropyl, CH₂-cyclobutyl, CH₂CH₂CH₂OH, CH₂CH₂CH₂OMe, or CH₂CH₂OH.

### R₁₀ₐ in Formulae I-II Compounds:

1. Lₐ is C₂₋₆ alkenylene

In an embodiment of the compounds of formulae I and II, Lₐ of -Lₐ-L_{b}-L_{c}-L_{d} is C₂₋₆ alkenylene. In another embodiment, Lₐ of -Lₐ-L_{b}-L_{c}-L_{d} is C₂₋₆ alkenylene, L_{b} and L, are absent, and L_{d} is H. In another embodiment, Lₐ is —CH₂═CH₂—, L_{b} and L, are absent, and L_{d} is H.

2. Lₐ is C₁₋₆ alkylene or Oxo

In an embodiment, Lₐ is C₁₋₆ alkylene. In further embodiments, Lₐ is —CH₂—, —CH₂CH₂—, —CH₂CH₂CH₂—, or —CH₂CH₂CH₂CH₂—. In a further embodiment of Lₐ, one methylene unit of —CH₂—, —CH₂CH₂—, —CH₂CH₂CH₂—, or —CH₂CH₂CH₂CH₂— can be replaced by oxo (C═O).

3. Lₐ is —CH₂— or Oxo

In one embodiment, Lₐ is CH₂ or oxo (C═O).

In another embodiment, Lₐ is CH₂ or C(═O); and L_{b} is absent.

In another embodiment, Lₐ is CH₂; and L_{b} is absent. In another embodiment, Lₐ is CH₂; L_{b} is absent; L, is CO, C(═O)NH—C₁₋₆ alkylene, —NH—C₁₋₆ alkylene, N(C₁₋₆ alkyl)-C₁₋₆ alkylene, and L_{d} is H, optionally substituted cycloalkyl, optionally substituted heterocycloalkyl optionally substituted aryl, optionally substituted heteroaryl or N(C₁₋₆ alkyl)₂. In another embodiment, Lₐ is CH₂; L_{b} is absent; L, is —NH—CH₂, N(C₁₋₆ alkyl)-C₁₋₆ alkylene, and L_{d} is H, C₁₋₆ alkyl, or optionally substituted heteroaryl. In a further embodiment, -Lₐ-L_{b}-L_{c}-L_{d} is CH₂—NHMe, CH₂—N(Me)-imidazolyl, CH₂—N(iPr)(Me), CH₂—N(Me)₂, CH₂—N(Et)₂, CH₂—N(iPr)(Me), CH₂—N(Me)(Et), CH₂—N(Me)(tBu), CH₂—N(H)(iPr), CH₂—N(Me)(cyclopropyl), or CH₂—N(Me)(CO)—CH₂—N(Me)₂.

3a. L_{b} is Optionally Substituted Cycloalkyl or Heterocycloalkyl

In another embodiment, Lₐ is CH₂ or oxo; and L_{b} is optionally substituted cycloalkyl or heterocycloalkyl. In another embodiment, Lₐ is CH₂ or oxo; and L_{b} is cyclobutyl, cyclopentyl, cyclohexyl, azetidinyl, pyrrolidinyl, piperidinyl, piperazinyl, or morpholinyl, any of which may be optionally substituted. In another embodiment, Lₐ is CH₂ or oxo; and L_{b} is cyclobutyl, azetidinyl, pyrrolidinyl, piperidinyl, piperazinyl, or morpholinyl, any of which may be optionally substituted.

In another embodiment, Lₐ is CH₂ or oxo; and L_{b} is cyclobutyl, azetidinyl, pyrrolidinyl, piperidinyl, piperazinyl, or morpholinyl, any of which may be optionally substituted. In these and other embodiments, L, is absent or is C₁₋₆ alkylene, C₁₋₆ alkylene-N(C₁₋₆ alkyl), NH, N—C₁₋₆ alkyl, N—C₁₋₆ cycloalkyl, —NH—C₁₋₆ alkylene, —N(C₁₋₆ alkyl)-C₁₋₆ alkylene, —N—C₁₋₆ alkylene-cycloalkyl, N—C₁₋₆ alkylene-heterocycloalkyl, N—C₁₋₆ alkylene-aryl, N—C₁₋₆ alkylene-heteroaryl, C(═O), C(═O)O, C(═O)NH, C(═O)N-alkyl, C(═O)NH—C₁₋₆ alkylene, C(═O)N(C₁₋₆ alkyl)-C₁₋₆ alkylene, SO₂, SO₂NH, SO₂N—C₁₋₆ alkyl, OC(═O)—NH, OC(═O)—N-alkyl, N(C₁₋₆ alkyl)-C(═O)—C₁₋₆ alkylene, SO₂, SO₂C₁₋₆ alkylene, SO₂NH, SO₂N(C₁₋₆ alkyl), SO₂NH—(C₁₋₆ alkylene), SO₂N(C₁₋₆ alkyl)-(C₁₋₆ alkylene), SO₂N(C₁₋₆ alkylene-aryl), SO₂N(C₁₋₆ alkylene-heteroaryl),

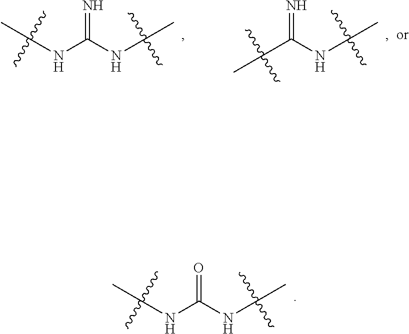 [SMILES: *NC(=N)N* ; *NC(*)=N ; *NC(=O)N*]

In another embodiment, Lₐ is CH₂ or oxo; and L_{b} is cyclobutyl, azetidinyl, piperidinyl, piperazinyl, pyrrolidinyl, or morpholinyl; L, is absent and L_{d} is H, —OH, C₁₋₆ alkyl, aryl, heteroaryl. In a further embodiment, Lₐ is CH₂, L_{b} is piperidinyl, piperazinyl, or morpholinyl; L, is absent and L_{d} is H, OH, methyl, trifluormethyl, ethyl, trifluoroethyl, propyl, isopropyl, butyl, isobutyl, t-butyl, pentyl, isopentyl, neopentyl, cyclopropyl, phenyl, 3-methoxyphenyl, 4-methoxyphenyl, 3-chlorophenyl, 4-chlorophenyl, 3-toluyl, 4-toluyl, 3-trifluoromethylphenyl, 4-trifluoromethylphenyl, naphthyl, imidazolyl, 2-pyridyl, 3-pyridyl, or 4-pyridyl,

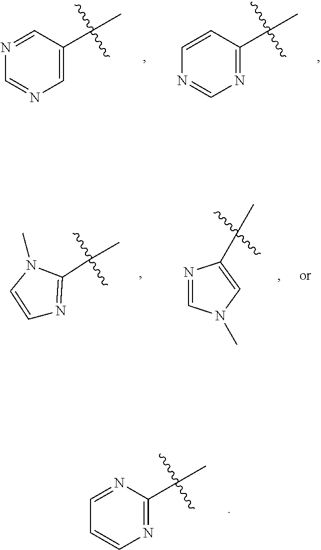

In another embodiment, Lₐ is CH₂ or oxo; and L_{b} is heterocycloalkyl or cycloalkyl. In a further embodiment, Lₐ is CH₂ or C(═O); L_{b} is piperidinyl, piperazinyl, pyrrolidinyl, or morpholinyl; L, is absent or is C₁₋₆ alkylene, C(═O), C(═O)O, C(═O)NH, C(═O)N-alkyl, C(═O)NH—C₁₋₆ alkylene, C(═O)N(C₁₋₆ alkyl)-C₁₋₆ alkylene; and L_{d} is H, —OH, C₁₋₆ alkyl, C₁₋₆ alkoxy, optionally substituted cycloalkyl, optionally substituted heterocycloalkyl, optionally substituted aryl, or optionally substituted heteroaryl.

In a further embodiment, Lₐ is CH₂ or C(═O); L_{b} is azetidinyl, piperidinyl, piperazinyl, or morpholinyl; L, is absent or is C₁₋₆ alkylene, C(═O), C(═O)O, C(═O)NH, C(═O)N(C₁₋₆ alkyl)-, C(═O)NH—C₁₋₆ alkylene, C(═O)N(C₁₋₆ alkyl)-C₁₋₆ alkylene, SO₂, SO₂C₁₋₆ alkylene, SO₂NH, or SO₂N(C₁₋₆ alkyl); and L_{d} is H, —OH, C₁₋₆ alkyl, C₁₋₆ alkoxy, C₁₋₆ haloalkyl, NH₂, NH(C₁₋₄ alkyl), N(C₁₋₄ alkyl)₂, optionally substituted C₃₋₆cycloalkyl, optionally substituted 3-6 membered heterocycloalkyl, optionally substituted C₆₋₁₂ aryl, or optionally substituted 5-10 membered heteroaryl.

In a further embodiment, Lₐ is CH₂ or oxo; L_{b} is cyclobutyl, piperidinyl, piperazinyl, pyrrolidinyl, or morpholinyl; L, is, C(═O), C(═O)NH, C(═O)N-alkyl, C(═O)NH— CHCH₃, C(═O)N(C₁₋₆ alkyl)-C₁₋₆ alkylene; and L_{d} is H, methyl, ethyl, isopropyl, isobutyl, t-butyl, hydroxy, methoxy, phenyl, 2-methoxyphenyl, 3-methoxyphenyl, 4-methoxyphenyl, 2-chlorophenyl, 3-chlorophenyl, 4-chlorophenyl, 2-toluyl, 3-toluyl, 4-toluyl, 2-trifluoromethylphenyl, 3-trifluoromethylphenyl, 4-trifluoromethylphenyl, 3-trifluoromethylphenyl, 4-trifluoromethylphenyl, naphthyl, imidazolyl, 2-pyridyl, 3-pyridyl, 4-pyridyl,

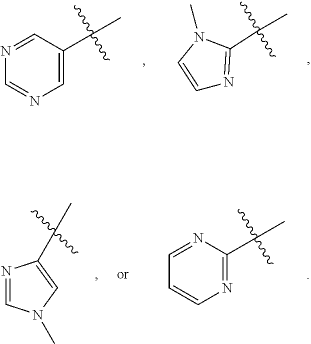

In another embodiment, LₐL_{b} is

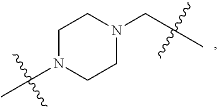 [SMILES: *CN1CCN(*)CC1]

L_{c} is absent, and L_{d} is H.

In another embodiment, LₐL_{b} is

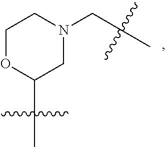 [SMILES: *CN1CCOC(*)C1]

L_{c} is absent, and L_{d} is H.

In another embodiment, LₐL_{b} is

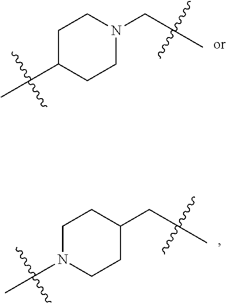 [SMILES: *CN1CCC(*)CC1 ; *CC1CCN(*)CC1]

L_{c} is absent, and L_{d} is H.

In another embodiment, LₐL_{b} is

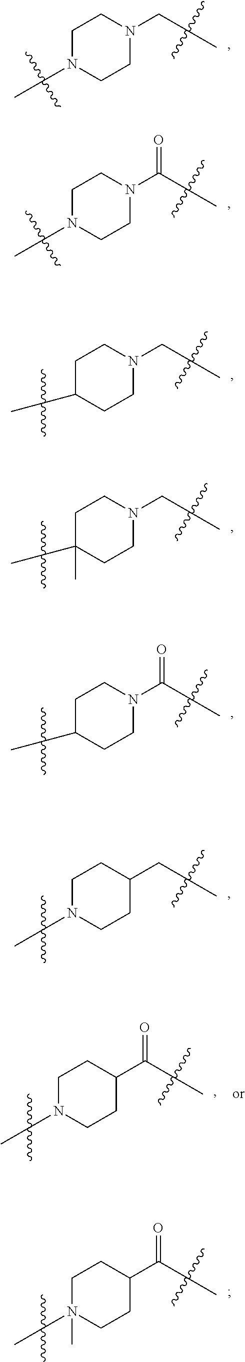

L_{c} is absent or is CH₂, CH₂CH₂, CHCH₃, CH₂CHCH₃, C(CH₃)₂, and L_{d} is methyl, isopropyl, tert-butyl, cyclopropyl, cyclobutyl, phenyl, trifluormethyl, 2-methoxyphenyl, 3-methoxyphenyl, 4-methoxyphenyl, 2-chlorophenyl, 3-chlorophenyl, 4-chlorophenyl, 2-toluyl, 3-toluyl, 4-toluyl, 2-trifluoromethylphenyl, 3-trifluoromethylphenyl, 4-trifluoromethylphenyl, 3-trifluoromethylphenyl, 4-trifluoromethylphenyl, naphthyl, imidazolyl, 2-pyridyl, 3-pyridyl, 4-pyridyl,

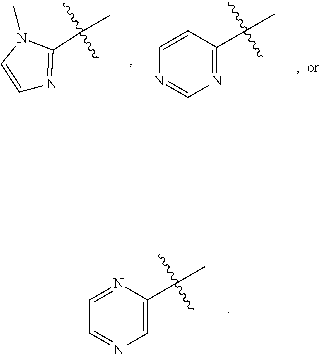

In another embodiment, LₐL_{b} is

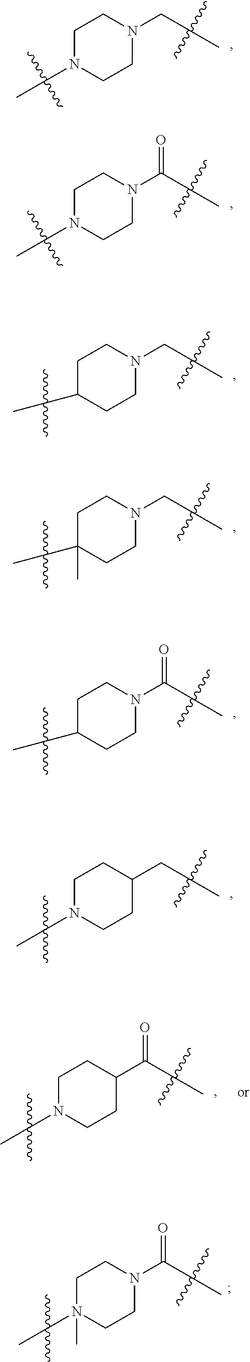

L_{c} is oxo, and L_{d} is methyl, ethyl, isopropyl, tert-butyl, cyclopropyl, phenyl, trifluormethyl, 2-methoxyphenyl, 3-methoxyphenyl, 4-methoxyphenyl, 2-chlorophenyl, 3-chlorophenyl, 4-chlorophenyl, 2-toluyl, 3-toluyl, 4-toluyl, 2-trifluoromethylphenyl, 3-trifluoromethylphenyl, 4-trifluoromethylphenyl, 3-trifluoromethylphenyl, 4-trifluoromethylphenyl, naphthyl, imidazolyl, 2-pyridyl, 3-pyridyl, 4-pyridyl,

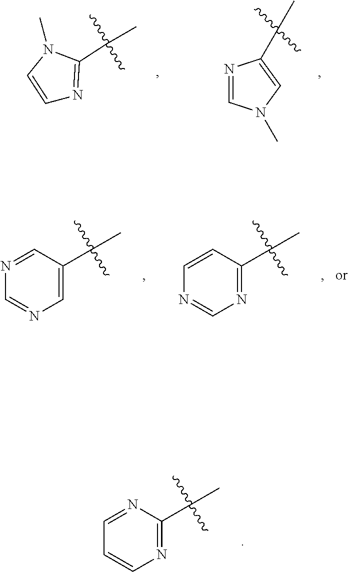

In another embodiment, LₐL_{b} is

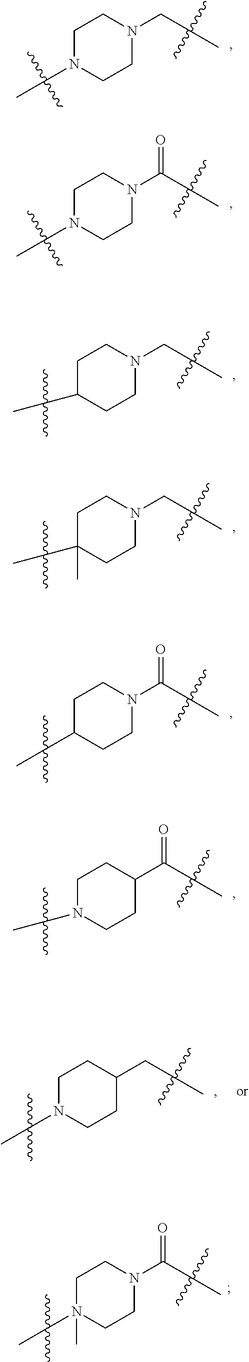

L_{c} is SO₂, SO₂ C₁₋₆ alkylene, SO₂NH, SO₂N—C₁₋₆ alkyl, SO₂N—(C₁₋₆ alkyl)(C₁₋₆ alkylene); and L_{d} is H, optionally substituted cycloalkyl, optionally substituted heterocycloalkyl optionally substituted aryl, optionally substituted heteroaryl. In a further embodiment, Lₐ is CH₂— or C(═O); L_{b} is cyclobutyl, piperidinyl, piperazinyl, pyrrolidinyl, or morpholinyl; L_{c} is SO₂, SO₂CH₂, SO₂ CHCH₃, SO₂NH, SO₂N—C₁₋₆ alkyl; and L_{d} is H, methyl, ethyl, propyl, isopropyl, hydroxyl, methoxy, phenyl, 2-methoxyphenyl, 3-methoxyphenyl, 4-methoxyphenyl, 2-chlorophenyl, 3-chlorophenyl, 4-chlorophenyl, 2-toluyl, 3-toluyl, 4-toluyl, 2-trifluoromethylphenyl, 3-trifluoromethylphenyl, 4-trifluoromethylphenyl, 3-trifluoromethylphenyl, 4-trifluoromethylphenyl, naphthyl, imidazolyl, 2-pyridyl, 3-pyridyl, 4-pyridyl,

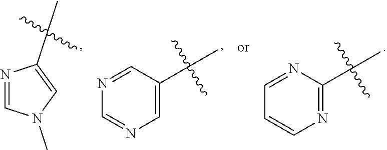

In another embodiment, LₐL_{b} is

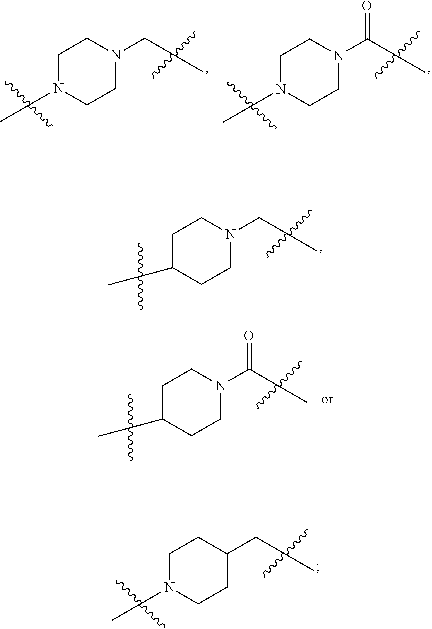 [SMILES: *CN1CCN(*)CC1 ; *C(=O)N1CCN(*)CC1 ; *CN1CCC(*)CC1 ; *C(=O)N1CCC(*)CC1 ; *CC1CCN(*)CC1]

L_{c} is absent or is C₁₋₆ alkylene, C(═O), C(═O)O, C(═O)NH, C(═O)N-alkyl, C(═O)NH—C₁₋₆ alkylene, C(═O)N(C₁₋₆ alkyl)-C₁₋₆ alkylene; and L_{d} is H, hydroxyl, C₁₋₆ alkyl, C₁₋₆ haloalkyl, alkoxy, optionally substituted cycloalkyl, optionally substituted heterocycloalkyl, optionally substituted aryl, optionally substituted heteroaryl. In a further embodiment, L, is absent or is C₁₋₆ alkylene, C(═O), C(═O)NH, C(═O)O, C(═O)N-alkyl, C(═O)NH—CH₂, C(═O)NH—CHCH₃, C(═O)N(C₁₋₆ alkyl)-C₁₋₆ alkylene; and L_{d} is H, methyl, ethyl, isopropyl, hydroxy, methoxy, trifluoromethyl, cyclopropyl, cyclobutyl, phenyl, 2-methoxyphenyl, 3-methoxyphenyl, 4-methoxyphenyl, 2-chlorophenyl, 3-chlorophenyl, 4-chlorophenyl, 2-toluyl, 3-toluyl, 4-toluyl, 2-trifluoromethylphenyl, 3-trifluoromethylphenyl, 4-trifluoromethylphenyl, 3-trifluoromethylphenyl, 4-trifluoromethylphenyl, naphthyl, imidazolyl, 2-pyridyl, 3-pyridyl, 4-pyridyl,

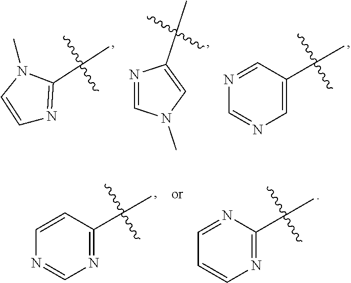

In another embodiment, Lₐ is CH₂ or oxo; L_{b} is cyclobutyl, L, is absent or is —NH—C₁₋₆ alkylene, N(C₁₋₆ alkyl)-C₁₋₆ alkylene, and L_{d} is H, optionally substituted cycloalkyl, optionally substituted heterocycloalkyl optionally substituted aryl, optionally substituted heteroaryl. In a further embodiment, Lₐ is CH₂ or C(═O); L_{b} is cyclobutyl; L, is absent or is CH₂, CH₂CH₂, or oxo; and L_{d} is H, methyl ethyl, isopropyl, cyclopropyl, imidazolyl, 2-pyridyl, 3-pyridyl, 4-pyridyl,

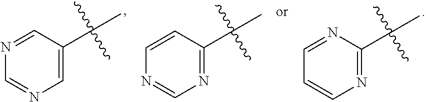 [SMILES: *c1cncnc1 ; *c1ccncn1 ; *c1ncccn1]

In a further embodiment, LₐL_{b} is

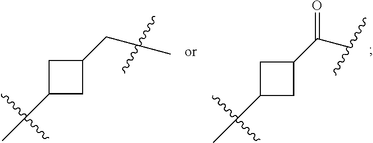 [SMILES: *CC1CC(*)C1 ; *C(=O)C1CC(*)C1]

L_{c} is NHCH₂—, N(Me)CH₂—, N(Et)CH₂—, N(iPr)CH₂—, N(isobutyl)CH₂—; and L_{d} is H or cyclopropyl. In a further embodiment, LₐL_{b} is

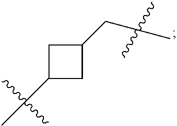 [SMILES: *CC1CC(*)C1]

L_{c} is NHCH₂—, N(Me)CH₂—, N(Et)CH₂—, N(iPr)CH₂—, N(isobutyl)CH₂—; and L_{d} is H or cyclopropyl.

In another embodiment the compound is a compound of formula A:

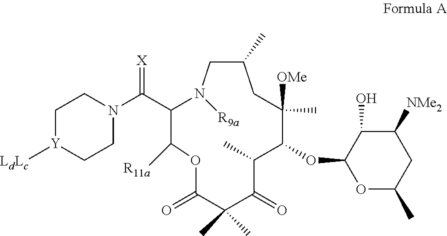

or a pharmaceutically acceptable salt thereof, wherein:
X is H₂ or O
Y is N, CH, C—(C₁₋₆ alkyl), or O;
R₉ₐ is selected from the group consisting of H, optionally substituted C₁₋₆ alkyl, C₁₋₆ alkylene-OH, C₁₋₆ alkylene-O—C₁₋₆ alkyl, C(═O)C₁₋₆ alkyl, C₁₋₆ alkylene-cyclolkyl, or C(═O)cycloalkyl;
L_{c} is absent or is C₁₋₄ alkylene, C₁₋₄ alkylene-N(C₁₋₄ alkyl), NH, N—C₁₋₄ alkyl, N—C₁₋₄ cycloalkyl, —NH—C₁₋₄ alkylene, —N(C₁₋₄ alkyl)-C₁₋₄ alkylene, —N—C₁₋₄ alkylene-cycloalkyl, N—C₁₋₄ alkylene-heterocycloalkyl, N—C₁₋₄ alkylene-aryl, N—C₁₋₄ alkylene-heteroaryl, C(═O), C(═O)O—C(═O)NH, C(═O)N-alkyl, C(═O)NH—C₁₋₄ alkylene, C(═O)N(C₁₋₄ alkyl)-C₁₋₄ alkylene SO₂, SO₂NH, SO₂N—C₁₋₄ alkyl, SO₂N—(C₁₋₄ alkyl)-(C₁₋₄ alkylene), OC(═O)—NH, OC(═O)—N—C₁₋₄ alkyl, N(C₁₋₆ alkyl)-C(═O)—C₁₋₆ alkylene, SO₂, SO₂C₁₋₄ alkylene, SO₂NH, SO₂N(C₁₋₄ alkyl), SO₂NH—(C₁₋₄ alkylene, SO₂N(C₁₋₄ alkyl)-(C₁₋₄ alkylene), SO₂N(C₁₋₄ alkylene-aryl), SO₂N(C₁₋₄ alkylene-heteroaryl),

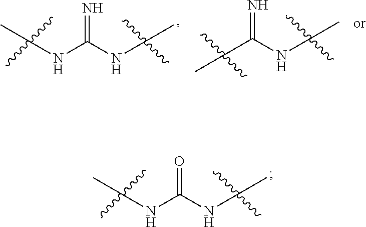 [SMILES: *NC(=N)N* ; *NC(*)=N ; *NC(=O)N*]

L_{d} is H, C₁₋₆ alkyl, OH, alkoxy, NH₂, NHC₁₋₆ alkyl, N(C₁₋₆ alkyl)₂, C₁₋₆ alkyl, C₁₋₆ haloalkyl, optionally substituted cycloalkyl, optionally substituted heterocycloalkyl optionally substituted aryl, optionally substituted heteroaryl, optionally substituted benzyl; and
R₁₁ₐ is H or C₁₋₆ alkyl.

In a further embodiment of Formula A, R₉ₐ, R₁₁ₐ, L_{c}, and L_{d}, have any of the defintions provided in Section 3a when L_{b} is optionally substituted heterocycloalkyl.

In a further embodiment of Formula A,
R₉ₐ is —H, methyl, ethyl, propyl, isopropyl, butyl, isobutyl, acetyl, C(═O)-ethyl, C(═O)-cyclopropyl, CH₂-cyclobutyl, CH₂CH₂CH₂OH, CH₂CH₂CH₂OMe, or CH₂CH₂OH;
R₁₁ₐ is H or methyl;
L_{c} is absent or is C₁₋₄ alkylene, NH, N—C₁₋₄ alkyl, —N(C₁₋₄ alkyl)-C₁₋₄ alkylene, C(═O), C(═O)O—, C(═O)NH, C(═O)N-alkyl, C(═O)NH—C₁₋₄ alkylene, OC(═O)—NH, SO₂, SO₂C₁₋₄ alkylene, SO₂NH, SO₂N(C₁₋₄ alkyl), or

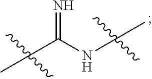 [SMILES: *NC(*)=N]

L_{d} is H, NH₂, NH(C₁₋₄ alkyl), N(C₁₋₄ alkyl)₂, methyl, ethyl, isopropyl, isobutyl, t-butyl, trifluoromethyl, hydroxy, methoxy, cyclopropyl, cyclobutyl, phenyl, 2-methoxyphenyl, 3-methoxyphenyl, 4-methoxyphenyl, 2-chlorophenyl, 3-chlorophenyl, 4-chlorophenyl, 2-toluyl, 3-toluyl, 4-toluyl, 2-trifluoromethylphenyl, 3-trifluoromethylphenyl, 4-trifluoromethylphenyl, 3-trifluoromethylphenyl, 4-trifluoromethylphenyl, naphthyl, imidazolyl, 2-pyridyl, 3-pyridyl, 4-pyridyl,

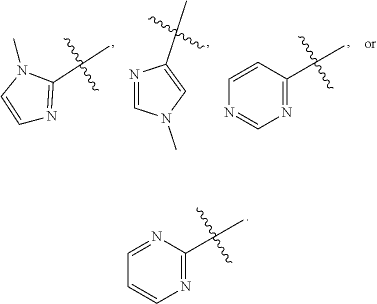

In a further embodiment of formula A:
X is H₂ or O
Y is N, CH, or O; and
R₁₁ₐ is H or methyl.

In another embodiment, a compound of formula A1 is provided:

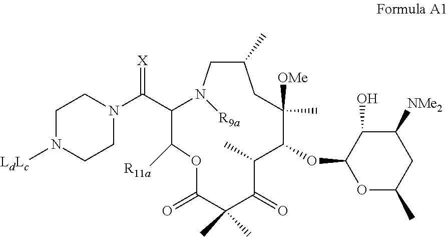

or a pharmaceutically acceptable salt thereof, wherein:
X is H₂ or O;
R₉ₐ is H or C₁₋₄alkyl;
R₁₁ₐ is H or C₁₋₄alkyl;
L_{c} is absent or is C₁₋₃ alkylene, C(═O), C(═O)NH, C(═O)NH—C₁₋₃ alkylene, or SO₂C₁₋₃ alkylene; and
L_{d} is NH(C₁₋₄ alkyl), N(C₁₋₄ alkyl)₂, C₃₋₆ cycloalkyl, 6-10 membered aryl or 5-10 membered heteroaryl, wherein the cycloalkyl, aryl, and heteroaryl are each independently and optionally substituted with halo, C₁₋₄alkyl, C₁₋₄haloalkyl, or C₁₋₄alkoxy.

In a further embodiment of formula A1:
R₉ₐ is H, Me, Et, or Pr;
R₁₁ₐ is H or Me;
L_{c} is CH₂, CH₂CH₂, C(═O)NH, C(═O), C(═O)NHCH₂, or SO₂CH₂; and
L_{d} is N(CH₃)₂, NHCH(CH₃)₂, cyclopropyl, cyclobutyl, phenyl, naphthalenyl, imidazolyl, pyrimidinyl, or pyridinyl, wherein phenyl, imidazolyl, pyrimidinyl, and pyridinyl are each independently and optionally substituted with F, Cl, Br, Me, OMe, or CF₃.

In another embodiment, the compound of formula A1 is not

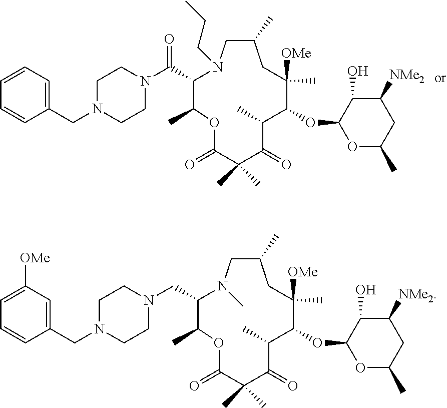

In another embodiment, a compound of formula A2 is provided:

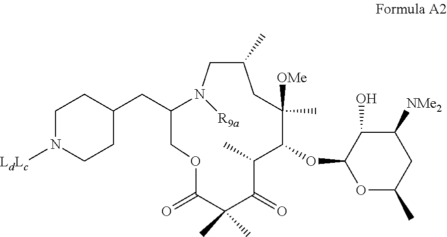

or a pharmaceutically acceptable salt thereof, wherein:
R₉ₐ is H or C₁₋₄alkyl;
L_{c} is C₁₋₃ alkylene, C(═O), C(═O)NH, C(═O)NH—C₁₋₃ alkylene, SO₂, or SO₂C₁₋₃alkylene; and
L_{d} is OH, NH(C₁₋₄ alkyl), N(C₁₋₄ alkyl)₂, 6-10 membered aryl or 5-10 membered heteroaryl, wherein the aryl and heteroaryl are each independently and optionally substituted with halo, C₁₋₄alkyl, C₁₋₄haloalkyl, or C₁₋₄alkoxy.

In a further embodiment of formula A2:
R₉ₐ is H, Me, Et, Pr, butyl, or isopropyl;
L_{c} is CH₂, CH₂CH₂, C(═O), C(═O)NH, C(═O)NHCH₂, SO₂, or SO₂CH₂; and
L_{d} is OH, N(CH₃)₂, NHCH(CH₃)₂, phenyl, or pyridinyl, wherein phenyl and pyridinyl are each independently and optionally substituted with F, Cl, Br, Me, OMe, or CF₃.

In a further embodiment of formula A2:
R₉ₐ is H, Me, Et, or Pr;
L_{c} is CH₂ or C(═O); and
L_{d} is phenyl or pyridinyl, wherein phenyl and pyridinyl are each independently and optionally substituted with F, Cl, Br, Me, OMe, or CF₃.

In another embodiment, the compound is a compound of formula B:

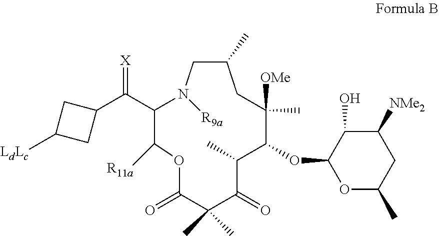

or a pharmaceutically acceptable salt thereof, wherein:
X is H₂ or O
R₉ₐ is selected from the group consisting of H, optionally substituted C₁₋₆ alkyl, C₁₋₆ alkylene-OH, C₁₋₆ alkylene-O-C₁₋₆ alkyl, C(═O)C₁₋₆ alkyl, C₁₋₆ alkylene-cyclolkyl, C(═O)cycloalkyl, or C(═O)NH-aryl;
L_{c} is absent or is C₁₋₄ alkylene, C₁₋₄ alkylene-N(C₁₋₄ alkyl), NH, N—C₁₋₄ alkyl, N—C₁₋₄ cycloalkyl, —NH—C₁₋₄ alkylene, —N(C₁₋₄ alkyl)-C₁₋₄ alkylene, —N—C₁₋₄ alkylene-cycloalkyl, N—C₁₋₄ alkylene-heterocycloalkyl, N—C₁₋₄ alkylene-aryl, N—C₁₋₄ alkylene-heteroaryl, C(═O), C(═O)O—C(═O)NH, C(═O)N-alkyl, C(═O)NH—C₁₋₄ alkylene, C(═O)N(C₁₋₄ alkyl)-C₁₋₄ alkylene SO₂, SO₂NH, SO₂N—C₁₋₄ alkyl, SO₂N—(C₁₋₄ alkyl)-(C₁₋₄ alkylene), OC(═O)—NH, OC(═O)—N—C₁₋₄ alkyl, SO₂, SO₂C₁₋₄ alkylene, SO₂NH, SO₂N(C₁₋₄ alkyl), SO₂NH—(C₁₋₄ alkylene, SO₂N(C₁₋₄ alkyl)-(C₁₋₄ alkylene), SO₂N(C₁₋₄ alkylene-aryl), SO₂N(C₁₋₄ alkylene-heteroaryl),

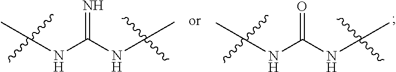 [SMILES: *NC(=N)N* ; *NC(=O)N*]

L_{d} is H, C₁₋₆ alkyl, OH, alkoxy, NH₂, NHC₁₋₆ alkyl, N(C₁₋₆ alkyl)₂, C₁₋₆ alkyl, C₁₋₆ haloalkyl, optionally substituted cycloalkyl, optionally substituted heterocycloalkyl optionally substituted aryl, optionally substituted heteroaryl, optionally substituted benzyl; and
R₁₁ₐ is H or C₁₋₆ alkyl.

In a further embodiment of Formula B, R₉ₐ, R₁₁ₐ, L_{c}, and L_{d}, have any of the defintions provided in Section 3a when L_{b} is optionally substituted cycloalkyl.

In a further embodiment of Formula B,
R₉ₐ is —H, methyl, ethyl, propyl, isopropyl, butyl, isobutyl, acetyl, C(═O)-ethyl, C(═O)-cyclopropyl, CH₂-cyclobutyl, CH₂CH₂CH₂OH, CH₂CH₂CH₂OMe, or CH₂CH₂OH;
R₁₁ₐ is H or methyl;
L_{c} is absent or is C₁₋₄ alkylene, NH, N—C₁₋₄ alkyl, or —N(C₁₋₄ alkyl)-C₁₋₄ alkylene; and
L_{d} is H, C₁₋₆ alkyl, or optionally substituted cycloalkyl.

In a further embodiment of formula B:
X is H₂ or O
R₉ₐ is selected from the group consisting of H, C₁₋₄ alkyl, C₁₋₄ alkylene-OH, C₁₋₄ alkylene-OMe and
R₁₁ₐ is H or methyl.

In another embodiment, R₁₀ₐ is selected from the group consisting of:

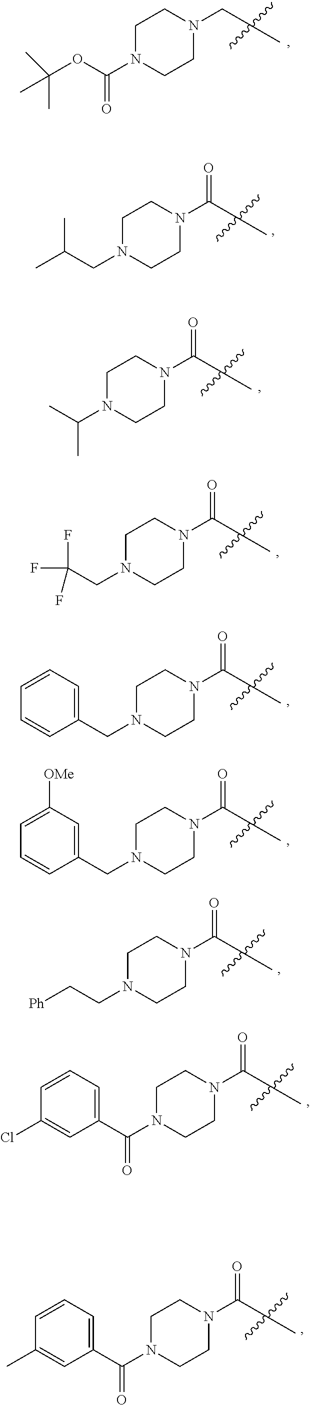

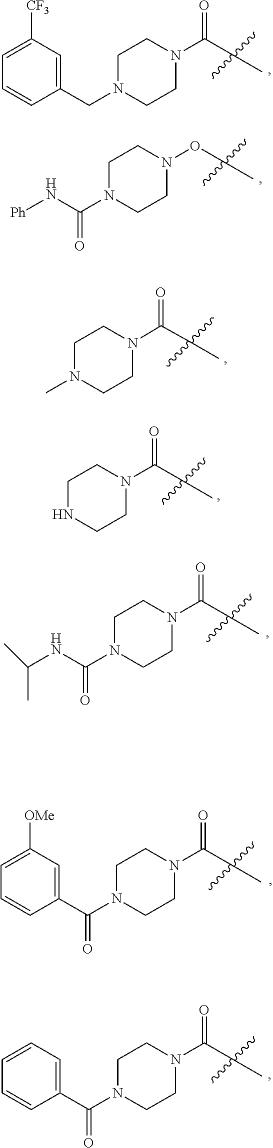

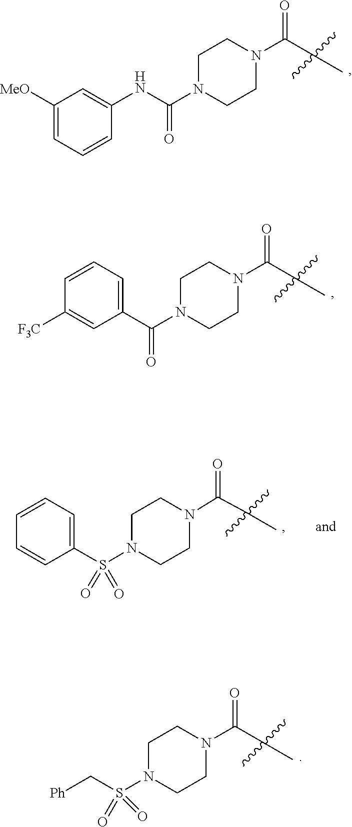 [SMILES: *C(=O)N1CCN(C(=O)Nc2cccc(OC)c2)CC1 ; *C(=O)N1CCN(C(=O)c2cccc(C(F)(F)F)c2)CC1 ; *C(=O)N1CCN(S(=O)(=O)c2ccccc2)CC1 ; *C(=O)N1CCN(S(=O)(=O)Cc2ccccc2)CC1]

In another embodiment, R₁₀ₐ is selected from the group consisting of:

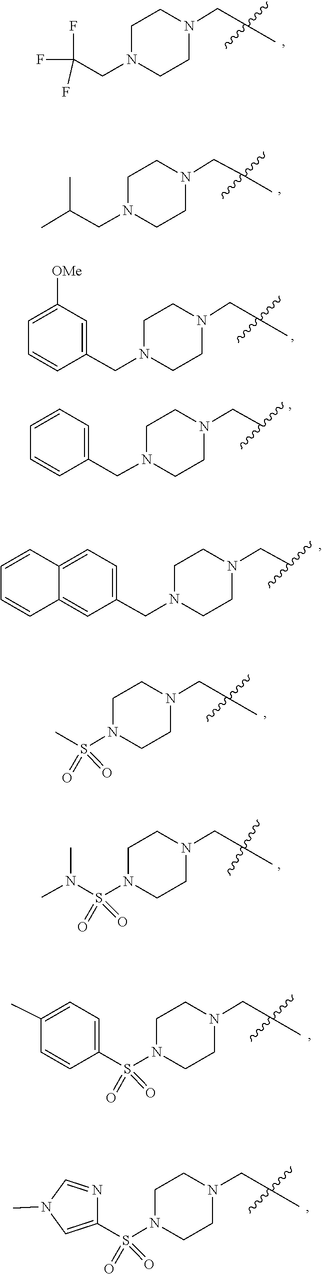

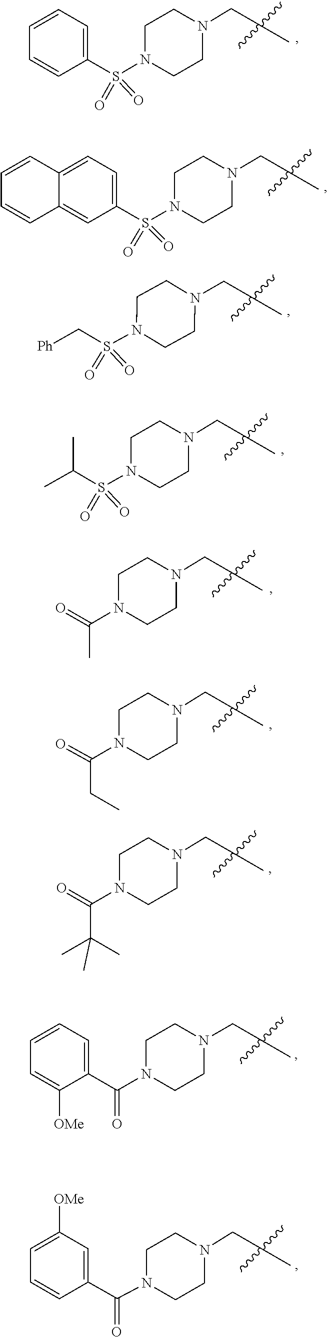

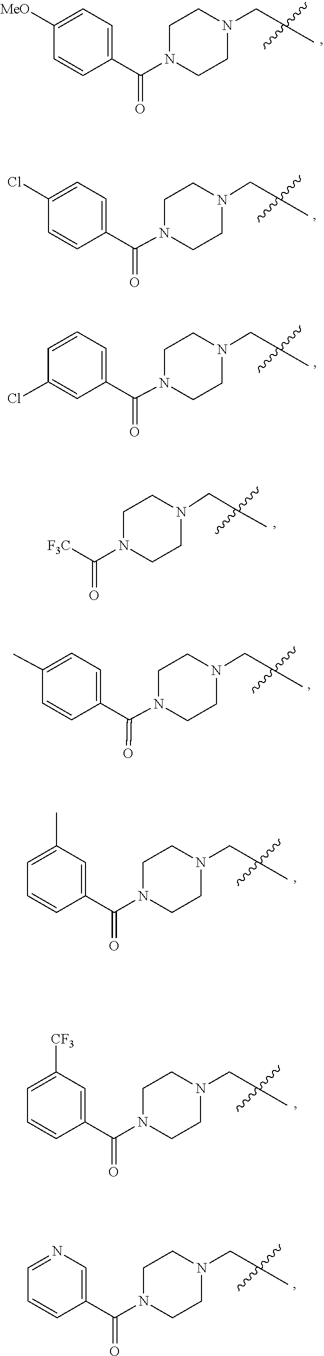

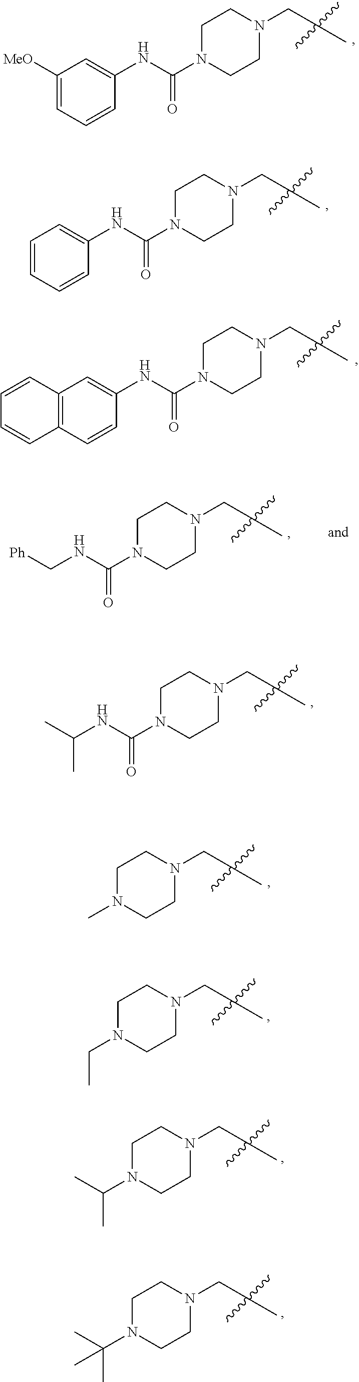

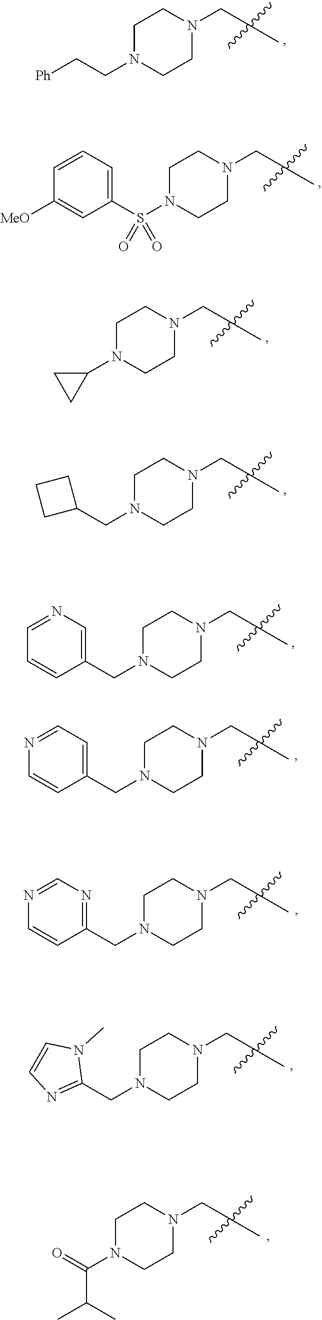

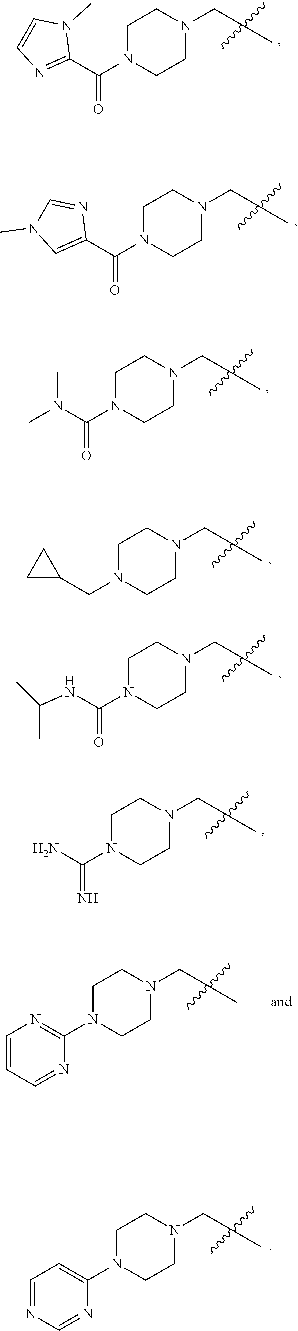

In another embodiment, R₁₀ₐ is selected from the group consisting of:

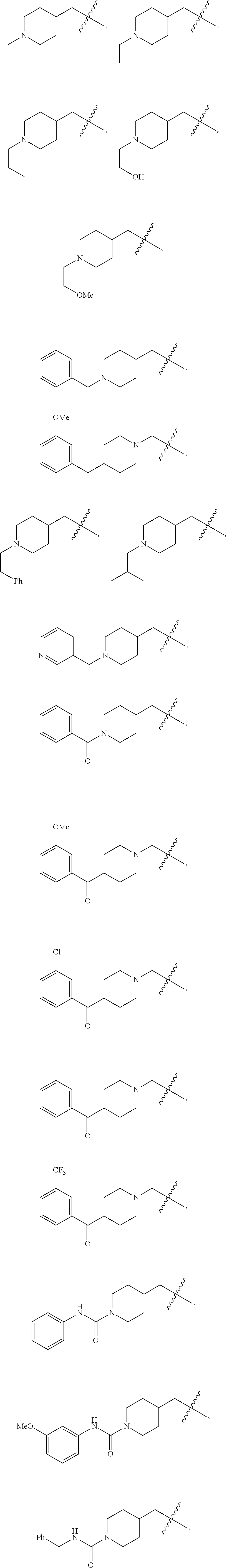

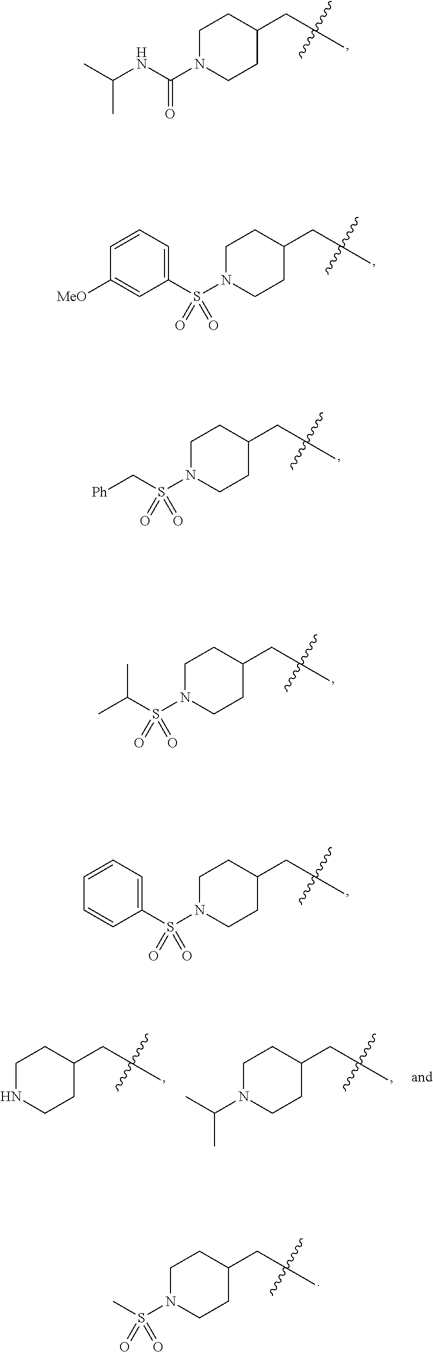

In another embodiment, R₁₀ₐ is selected from the group consisting of

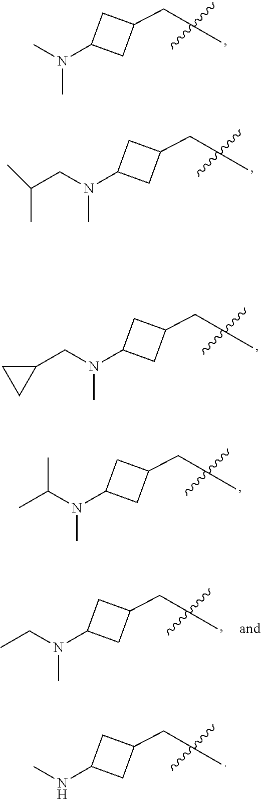

In another embodiment, R₁₀ₐ is selected from the group consisting of —CH₂NHME, —CH₂N(ME)₂,

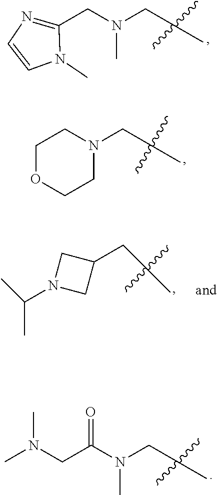

4. Lₐ is CH₂CH₂ or CH₂CH₂ CH₂

In an embodiment, Lₐ is CH₂CH₂ or CH₂CH₂CH₂. In another embodiment, one methylene unit of Lₐ can be replaced by oxo. In another embodiment, Lₐ COCH₂, COCH₂CH₂, CH₂COCH₂. In another embodiment, Lₐ is CH₂CH₂, CH₂CH₂CH₂, COCH₂, COCH₂CH₂, CH₂COCH₂; L_{b} is absent, or is optionally substituted cycloalkyl or optionally substituted heterocycloalkyl; L, is absent or is C₁₋₆ alkylene, C₁₋₆ alkylene-N(C₁₋₆ alkyl), NH, N—C₁₋₆ alkyl, N—C₁₋₆ cycloalkyl, —NH—C₁₋₆ alkylene, —NH—C₁₋₆ alkylene-heteroarylene, —N(C₁₋₆ alkyl)-C₁₋₆ alkylene, —N—C₁₋₆ alkylene-cycloalkyl, N—C₁₋₆ alkylene-heterocycloalkyl, N—C₁₋₆ alkylene-aryl, N—C₁₋₆ alkylene-heteroaryl, C(═O), C(═O)O—, C(═O)NH, C(═O)N-alkyl, C(═O)NH—C₁₋₆ alkylene, C(═O)N(C₁₋₆ alkyl)-C₁₋₆ alkylene SO₂, SO₂ C₁₋₆ alkylene, SO₂NH, SO₂N—C₁₋₆ alkyl, OC(═O)—NH, OC(═O)—N-alkyl, SO₂, SO₂C₁₋₆ alkylene, SO₂NH, SO₂N(C₁₋₆ alkyl), SO₂NH—(C₁₋₆ alkylene, SO₂N(C₁₋₆ alkyl)-(C₁₋₆ alkylene), SO₂N(C₁₋₆ alkylene-aryl), SO₂N(C₁₋₆ alkylene-heteroaryl),

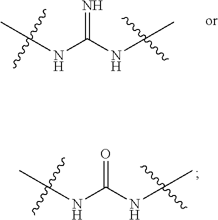 [SMILES: *NC(=N)N* ; *NC(=O)N*]

and L_{d} is H, C₁₋₆ alkyl, OH, alkoxy, NH₂, NHC₁₋₆ alkyl, N(C₁₋₆ alkyl)₂, C₁₋₆ alkyl, C₁₋₆ haloalkyl, optionally substituted cycloalkyl, optionally substituted heterocycloalkyl optionally substituted aryl, or optionally substituted heteroaryl.

In another embodiment, Lₐ is CH₂CH₂CH₂, and L_{b} and L, are absent.

In one embodiment, Lₐ is CH₂CH₂, CH₂CH₂CH₂, COCH₂, COCH₂CH₂, CH₂COCH₂; L_{b} is absent, L, is N(C₁₋₆ alkyl), NH, N—C₁₋₆ alkyl, N—C₁₋₆ cycloalkyl, —NH—C₁₋₆ alkylene, —N(C₁₋₆ alkyl)-C₁₋₆ alkylene, —N—C₁₋₆ alkylene-cycloalkyl, N—C₁₋₆ alkylene-heterocycloalkyl, N—C₁₋₆ alkylene-aryl, or N—C₁₋₆ alkylene-heteroaryl; and L_{d} is H, OH, C₁₋₆ alkyl, OH, alkoxy, NH₂, NHC₁₋₆ alkyl, N(C₁₋₆ alkyl)₂, C₁₋₆ alkyl, C₁₋₆ haloalkyl, optionally substituted cycloalkyl, optionally substituted heterocycloalkyl optionally substituted aryl, or optionally substituted heteroaryl. In another embodiment, Lₐ is CH₂CH₂, CH₂CH₂CH₂, COCH₂, COCH₂CH₂, CH₂COCH₂; L_{b} is absent; L, is N(Me), N(Et), N(Me)(CH₂), NH; and L_{d} is H, methyl, ethyl, isopropyl, tert-butyl, cyclopropyl, cyclobutyl, cyclopentyl, or cyclohexyl.

In another embodiment, Lₐ is CH₂CH₂, CH₂CH₂CH₂, COCH₂, COCH₂CH₂, CH₂COCH₂; L_{b} is absent; L, is absent; and L_{d} is pyrrolidinyl, oxazolyl,

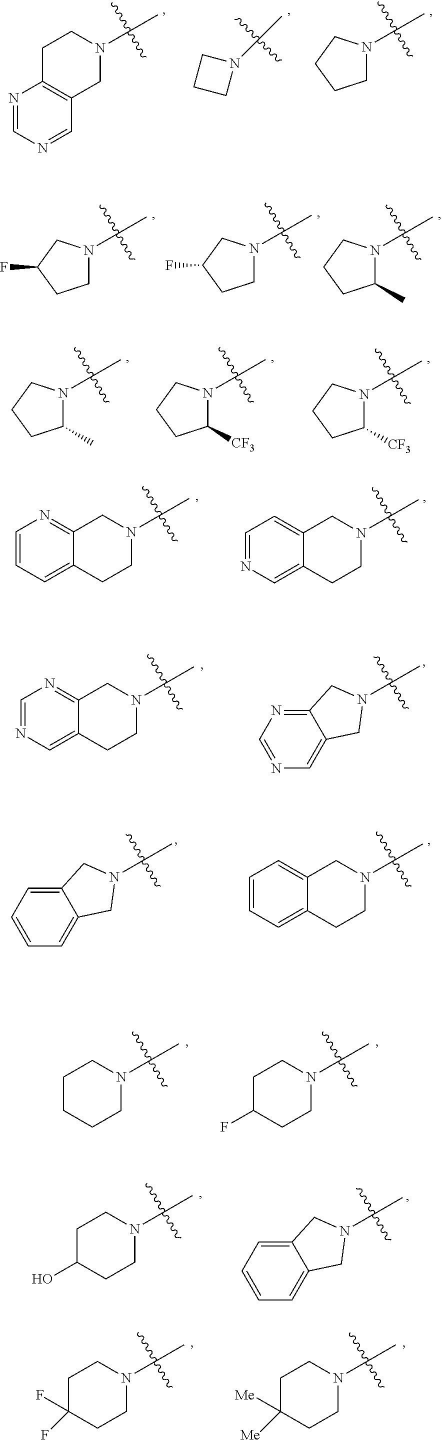

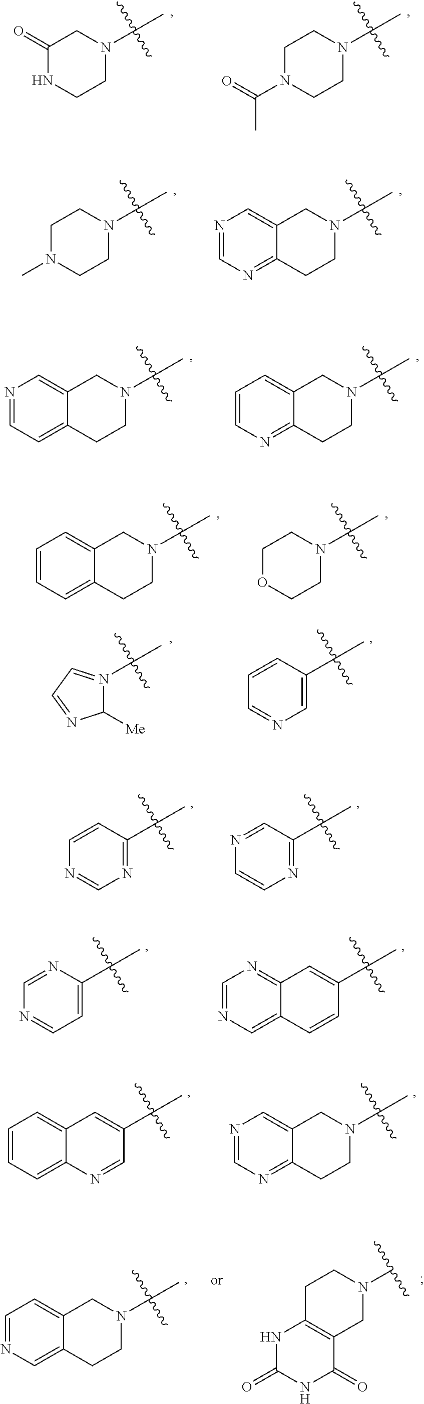

where [symbol] indicates a point of attachment.

In one embodiment, Lₐ is CH₂CH₂, CH₂CH₂CH₂, COCH₂, COCH₂CH₂, CH₂COCH₂; L_{b} is absent, L, is N(C₁₋₆ alkyl), NH, N—C₁₋₆ alkyl, N—C₁₋₆ cycloalkyl, —NH—C₁₋₆ alkylene, —NH—C₁₋₆ alkylene-heteroarylene, —N(C₁₋₆ alkyl)-C₁₋₆ alkylene, —N—C₁₋₆ alkylene-cycloalkyl, N—C₁₋₆ alkylene-heterocycloalkyl, N—C₁₋₆ alkylene-aryl, or N—C₁₋₆ alkylene-heteroaryl; and L_{d} is H, OH, C₁₋₆ alkyl, OH, alkoxy, NH₂, NHC₁₋₆ alkyl, N(C₁₋₆ alkyl)₂, C₁₋₆ alkyl, C₁₋₆ haloalkyl, optionally substituted cycloalkyl, optionally substituted heterocycloalkyl optionally substituted aryl, or optionally substituted heteroaryl.

In another embodiment, Lₐ is CH₂CH₂CH₂, L_{b} is absent; L, is N(Me), N(Et), N(Me)(CH₂), NH; and L_{d} is H, methyl, ethyl, isopropyl, tert-butyl, cyclopropyl, cyclobutyl, cyclopentyl, cyclohexyl, pyrrolidinyl, oxazolyl, imidazolyl. In another embodiment, LₐL_{b} is CH₂CH₂CH₂, and L_{c}L_{d} is NH₂, NH-Me, NH-Et, NH-isopropyl, NH-cyclopropyl, NH-cyclobutyl, NH-cyclopentyl, N(Me)₂, N(Et)₂, N(Me)(Et), N(Me)-cyclopropyl, N(Me)-cyclobutyl, N(Me)-cyclopentyl, N(Me)CH₂-imidazolyl, N(Me)(iPr), N(Me)(tBu), NH-cyclopropyl, NH-oxazolyl, NH-pyrimidinyl, NH-pyridyl, NHCH₂-cyclopropyl, NHCH₂-oxazolyl, NHCH₂-pyrimidinyl, NHCH₂-pyridyl, NHCH₂-quinazolinyl, NHCH₂-quinolinyl, or NHCH₂-oxadiazolene-phenyl.

In another embodiment, Lₐ is CH₂CH₂CH₂, L_{b} is absent; L_{c} is absent; and L_{d} is OH or alkoxy.

In another embodiment, Lₐ is CH₂CH₂CH₂, L_{b} is absent; L, is N(Me), N(Et), N(Me)(CH₂), NH; and L_{d} is H, methyl, ethyl, isopropyl, tert-butyl, cyclopropyl, cyclobutyl, cyclopentyl, cyclohexyl, pyrrolidinyl, oxazolyl, imidazolyl. In another embodiment, LₐL_{b} is CH₂CH₂CH₂, and L_{c}L_{d} is NH₂, NHMe, NHEt, NHcyclopropyl, N(H)cyclobutyl, N(H)cyclopentyl, N(Me)₂, N(Et)₂, N(Me)(Et), N(Me)cyclopropyl, N(Me)cyclobutyl, N(Me)cyclopentyl, N(Me)CH₂-imidazolyl, N(Me)(iPr), N(Me)(tBu), NH-cyclopropyl, NH-oxazolyl, NH-pyrimidinyl, NH-pyridyl, NHCH₂-cyclopropyl, NHCH₂-oxazolyl, NHCH₂-pyrimidinyl, NHCH₂-pyridyl.

In an embodiment, Lₐ is CH₂CH₂CH₂; L_{b} is absent; L, is CO, C(═O)O—, OC(═O)—NH, C(═O)NH, or C(═O)NHCH₂; and L_{d} is H, C₁₋₆ alkyl, or optionally substuted aryl or heteroaryl. In another embodiment, Lₐ is CH₂CH₂CH₂; L_{b} is absent; L, is CO, and L_{d} is methyl. In another embodiment, LₐL_{b} is CH₂CH₂CH₂; and L_{c}L_{d} is O—C(═O)NH-phenyl, or

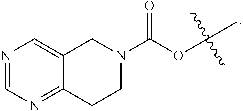 [SMILES: *OC(=O)N1CCc2ncncc2C1]

In another embodiment, Lₐ is CH₂CH₂CH₂: L_{b} is optionally substituted cycloalkyl or heterocycloalkyl; L_{c} is absent; and L_{d} is H, OH, C₁₋₆ alkyl, C₁₋₆ haloalkyl, optionally substituted aryl or optionally substituted heteroaryl.

In another embodiment, Lₐ is CH₂CH₂CH₂; L_{b} is absent; L_{c} is absent; and L_{d} is optionally substituted azetidinyl, optionally substituted pyrrolidinyl, optionally substituted piperidinyl, optionally substituted piperazinyl, optionally substituted morpholinyl; In another embodiment, L_{d} is H, methyl, trifluoromethyl, phenyl, pyridyl, pyrimidinyl, or OH. In another embodiment, Lₐ is CH₂CH₂CH₂, L_{b} is absent; L_{c} is absent; and L_{d} is azetidinyl, pyrrolidinyl, piperidinyl, piperazinyl,

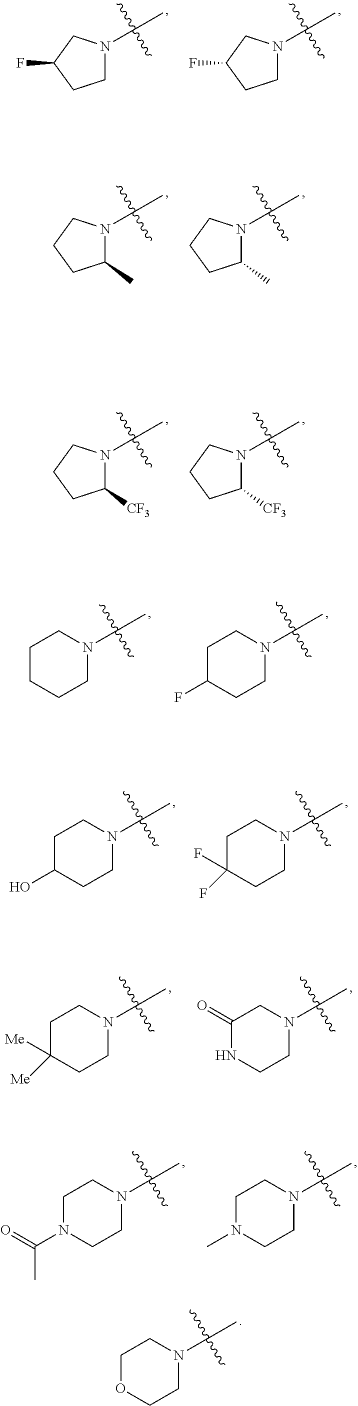

In another embodiment, Lₐ is CH₂CH₂CH₂, L_{b} is absent; L_{c} is absent; and methyl, trifluoromethyl, phenyl, pyridyl, pyrimidinyl, OH,

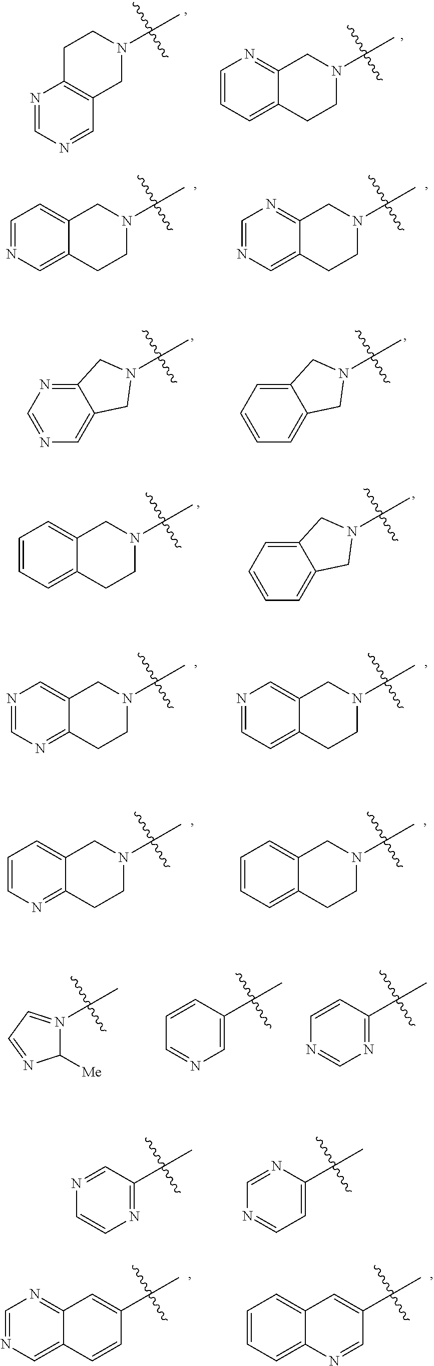

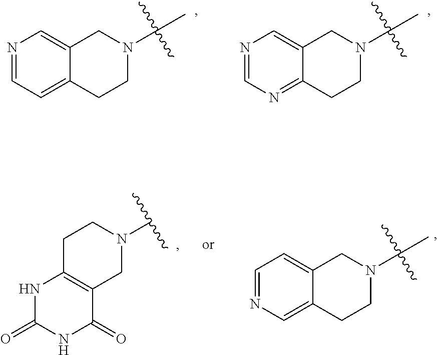 [SMILES: *N1CCc2ccncc2C1 ; *N1CCc2ncncc2C1 ; *N1CCc2[nH]c(=O)[nH]c(=O)c2C1 ; *N1CCc2cnccc2C1]

where [symbol] indicates a point of attachment.

In another embodiment, Lₐ is COCH₂CH₂ L_{b} is absent; L_{c} is absent; and L_{d} is phenyl, pyridyl, pyrimidinyl, OH,

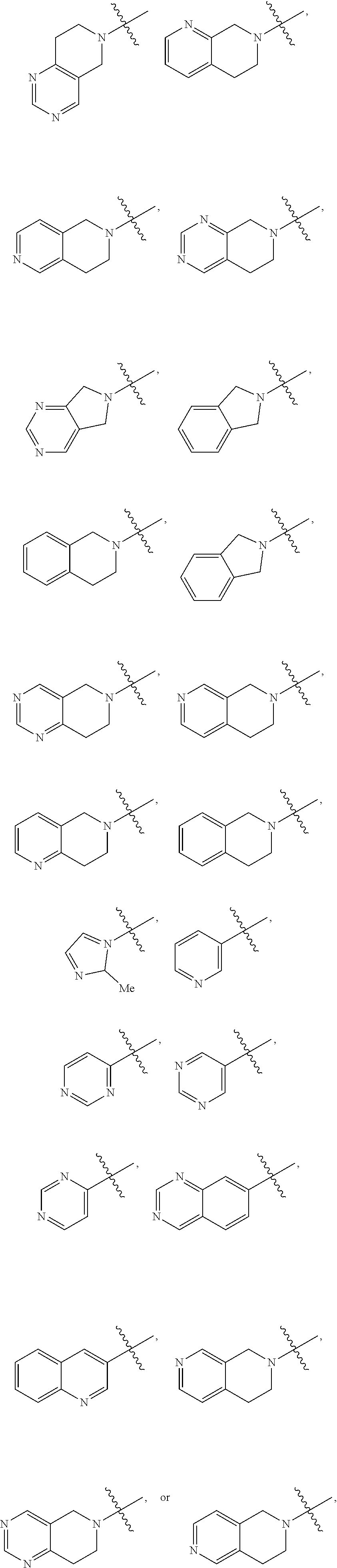

where [symbol] indicates a point of attachment.

In an embodiment, Lₐ is COCH₂CH₂; L_{b} is absent; L, is C₁₋₆ alkylene, C₁₋₆ alkylene-N(C₁₋₆ alkyl), NH, N—C₁₋₆ alkyl, N—C₁₋₆ cycloalkyl, —NH—C₁₋₆ alkylene, —N(C₁₋₆ alkyl)-C₁₋₆ alkylene, —N—C₁₋₆ alkylene-cycloalkyl, N—C₁₋₆ alkylene-heterocycloalkyl, N—C₁₋₆ alkylene-aryl, N—C₁₋₆ alkylene-heteroaryl, C(═O), C(═O)O—, C(═O)NH, C(═O)N-alkyl, C(═O)NH—C₁₋₆ alkylene, C(═O)N(C₁₋₆ alkyl)-C₁₋₆ alkylene SO₂, SO₂ C₁₋₆ alkylene, SO₂NH, SO₂N—C₁₋₆ alkyl, OC(═O)—NH, OC(═O)—N-alkyl, SO₂, SO₂C₁₋₆ alkylene, SO₂NH, SO₂N(C₁₋₆ alkyl), SO₂NH—(C₁₋₆ alkylene, SO₂N(C₁₋₆ alkyl)-(C₁₋₆ alkylene), SO₂N(C₁₋₆ alkylene-aryl), SO₂N(C₁₋₆ alkylene-heteroaryl),

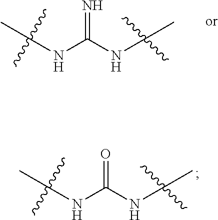 [SMILES: *NC(=N)N* ; *NC(=O)N*]

and L_{d} is H, C₁₋₆ alkyl, OH, alkoxy, NH₂, NHC₁₋₆ alkyl, N(C₁₋₆ alkyl)₂, C₁₋₆ alkyl, C₁₋₆ haloalkyl, optionally substituted cycloalkyl, optionally substituted heterocycloalkyl optionally substituted aryl, optionally substituted heteroaryl.

In an embodiment, Lₐ is COCH₂CH₂; L_{b} is absent; L_{c} is NH or NHCH₂; L_{d} is H, C₁₋₆ alkyl, OH, alkoxy, NH₂, NHC₁₋₆ alkyl, N(C₁₋₆ alkyl)₂, C₁₋₆ alkyl, C₁₋₆ haloalkyl, optionally substituted cycloalkyl, optionally substituted heterocycloalkyl optionally substituted aryl, optionally substituted heteroaryl.

In an embodiment, Lₐ is COCH₂CH₂; L_{b} is absent; L, is NH or NHCH₂; and L_{d} is optionally substituted pyrimidinyl, optionally substituted quinolinyl, optionally substituted oxazolyl, optionally substituted cyclobutyl.

In another embodiment, R₁₀ₐ is selected from the group consisting of HO—CH₂—CH₂—CH₂—, (Me)₂N—CH₂—CH₂—CH₂—, (Me)N—CH₂—CH₂—CH₂—, (Et)₂N—CH₂—CH₂—CH₂—,

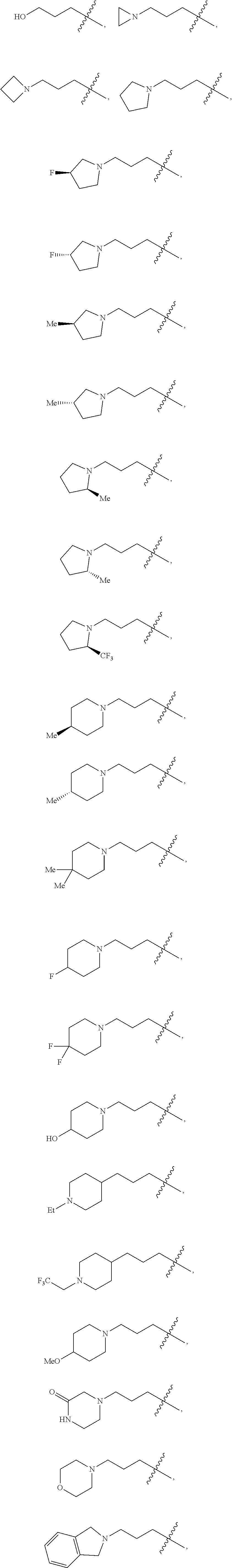

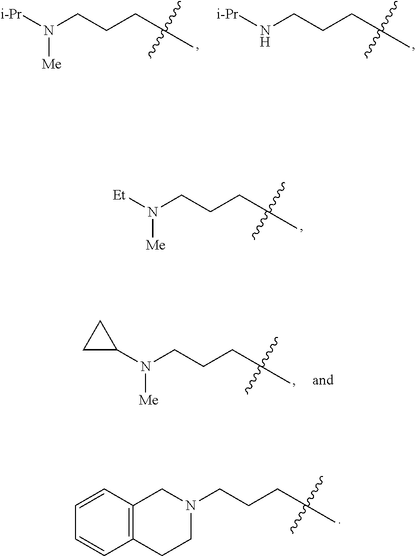 [SMILES: *CCCN(C)C(C)C ; *CCCNC(C)C ; *CCCN(C)CC ; *CCCN(C)C1CC1 ; *CCCN1CCc2ccccc2C1]

In another embodiment, R₁₀ₐ is selected from the group consisting of:

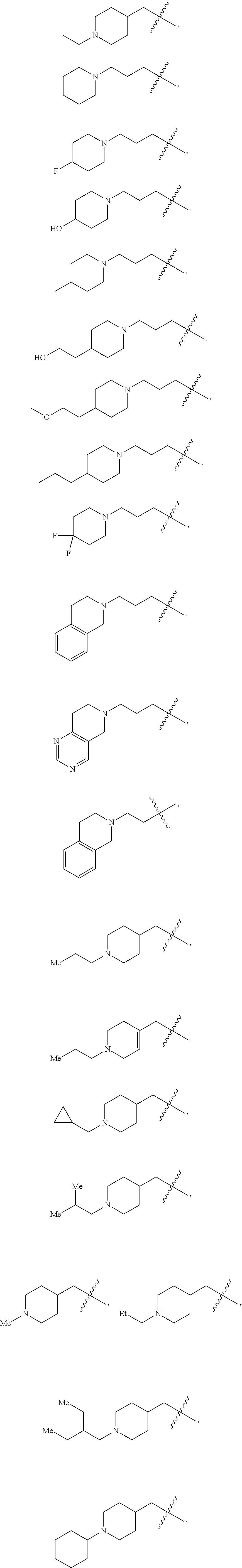

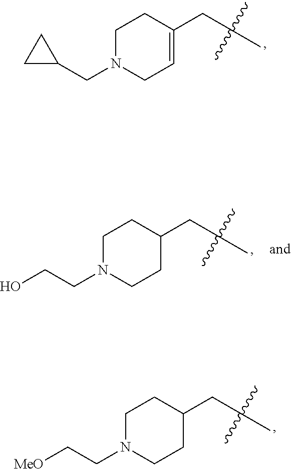 [SMILES: *CC1=CCN(CC2CC2)CC1 ; *CC1CCN(CCO)CC1 ; *CC1CCN(CCOC)CC1]

wherein [symbol] indicates a point of attachment.

In another embodiment the compound is a compound of formula C:

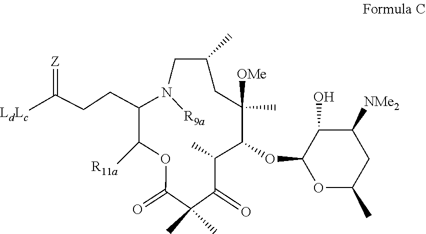

or a pharmaceutically acceptable salt thereof, wherein:
Z is H₂ or O;
R₉ₐ is selected from the group consisting of H, optionally substituted C₁₋₆ alkyl, C₁₋₆ alkylene-OH, C₁₋₆ alkylene-O—C₁₋₆ alkyl, C(═O)C₁₋₆ alkyl, C₁₋₆ alkylene-cyclolkyl, C(═O)cycloalkyl, or C(═O)NH-aryl;
L_{c} is absent or is C₁₋₄ alkylene, C₁₋₄ alkylene-N(C₁₋₄ alkyl), NH, N—C₁₋₄ alkyl, N—C₁₋₄ cycloalkyl, —NH—C₁₋₄ alkylene, —NH—C₁₋₆ alkylene-heteroarylene, —N(C₁₋₄ alkyl)-C₁₋₄ alkylene, —N—C₁₋₄ alkylene-cycloalkyl, N—C₁₋₄ alkylene-heterocycloalkyl, N—C₁₋₄ alkylene-aryl, N—C₁₋₄ alkylene-heteroaryl, C(═O), C(═O)O— C(═O)NH, C(═O)N-alkyl, C(═O)NH—C₁₋₄ alkylene, C(═O)N(C₁₋₄ alkyl)-C₁₋₄ alkylene SO₂, SO₂NH, SO₂N—C₁₋₄ alkyl, SO₂N—(C₁₋₄ alkyl)-(C₁₋₄ alkylene), OC(═O)—NH, OC(═O)—N—C₁₋₄ alkyl, SO₂, SO₂C₁₋₄ alkylene, SO₂NH, SO₂N(C₁₋₄ alkyl), SO₂NH—(C₁₋₄ alkylene, SO₂N(C₁₋₄ alkyl)-(C₁₋₄ alkylene), SO₂N(C₁₋₄ alkylene-aryl), SO₂N(C₁₋₄ alkylene-heteroaryl),

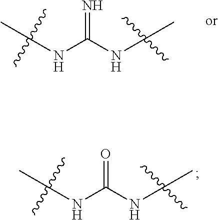 [SMILES: *NC(=N)N* ; *NC(=O)N*]

L_{d} is H, C₁₋₆ alkyl, OH, alkoxy, NH₂, NHC₁₋₆ alkyl, N(C₁₋₆ alkyl)₂, C₁₋₆ alkyl, C₁₋₆ haloalkyl, optionally substituted cycloalkyl, optionally substituted heterocycloalkyl optionally substituted aryl, optionally substituted heteroaryl, optionally substituted benzyl; and
R₁₁ₐ is H or C₁₋₆ alkyl.

In a further embodiment of Formula C, R₉ₐ, R₁₁ₐ, L_{c}, and L_{d}, have any of the defintions provided in Section 4.

In a further embodiment of Formula C, R₉ₐ is C₁₋₆ alkylene-O—C₁₋₆ alkyl, C(═O)C₁₋₆ alkyl, C₁₋₆ alkylene-cyclolkyl, C(═O)cycloalkyl, or C(═O)NH-aryl. In another embodiment, R₉ₐ is C₁₋₆ alkylene-O—C₁₋₆ alkyl, C(═O)C₁₋₆ alkyl, C₁₋₆ alkylene-C₁₋₆ cyclolkyl, C(═O)C₃₋₆ cycloalkyl, or C(═O)NH-aryl. In another embodiment, R₉ₐ is C₁₋₆ alkylene-OMe, C(═O)C₁₋₃alkyl, C(═O)C₃₋₆ cycloalkyl, or C(═O)NH-phenyl.

In a further embodiment of Formula C,
R₉ₐ is —H, methyl, ethyl, propyl, isopropyl, butyl, isobutyl, pentyl, isopentyl, acetyl, C(═O)—NH-phenyl, C(═O)-ethyl, C(═O)-cyclopropyl, CH₂-cyclobutyl, CH₂CH₂CH₂OH, CH₂CH₂CH₂OMe, or CH₂CH₂OH;
L_{c} is absent or is C₁₋₄ alkylene, C₁₋₄ alkylene-N(C₁₋₄ alkyl), NH, N—C₁₋₄ alkyl, N—C₁₋₄ cycloalkyl, —NH—C₁₋₄ alkylene, —N(C₁₋₄ alkyl)-C₁₋₄ alkylene, —N—C₁₋₄ alkylene-cycloalkyl, N—C₁₋₄ alkylene-heterocycloalkyl, N—C₁₋₄ alkylene-aryl, N—C₁₋₄ alkylene-heteroaryl, C(═O), C(═O)O—, C(═O)NH, C(═O)N-alkyl, C(═O)NH—C₁₋₄ alkylene, C(═O)N(C₁₋₄ alkyl)-C₁₋₄ alkylene, or OC(═O)—NH;
L_{d} is H, C₁₋₆ alkyl, OH, alkoxy, C₁₋₆ haloalkyl, optionally substituted cycloalkyl, optionally substituted heterocycloalkyl optionally substituted aryl, optionally substituted heteroaryl.

In a further embodiment of formula C:
Z is H₂ or O;
R₉ₐ is —H, methyl, ethyl, propyl, isopropyl, butyl, isobutyl, isopentyl, acetyl, C(═O)—NH-phenyl, C(═O)-ethyl, C(═O)-cyclopropyl, CH₂-cyclobutyl, or CH₂CH₂OH; and
R₁₁ₐ is H or methyl.

In another embodiment, the compound is a compound of formula C1:

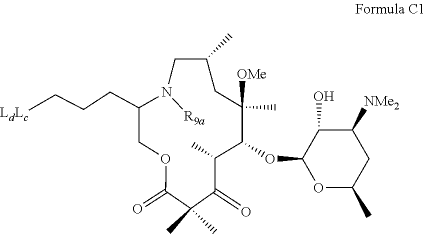

or a pharmaceutically acceptable salt thereof, wherein:
R₉ₐ is selected from the group consisting of H, C₁₋₆ alkyl, C₁₋₆ alkylene-OH, C₁₋₆ alkylene-O—C₁₋₆ alkyl, C(═O)C₁₋₆ alkyl, C₁₋₆ alkylene-cyclolkyl, C(═O)cycloalkyl, and C(═O)NH-aryl;
L_{c} is NH, NH—C₁₋₄ alkylene, NH—C₁₋₄ alkylene-(5-10 membered heteroarylene), C(═O)O—, C(═O)NH, or OC(═O)—NH; and
L_{d} is C₃₋₆ cycloalkyl, 6-10 membered aryl, or 5-10 memebred heteroaryl, wherein the cycloalkyl, aryl, and heteroaryl are each independently and optionally substituted with halo, C₁₋₄alkyl, C₁₋₄haloalkyl, or C₁₋₄alkoxy.

In a further embodiment of formula C1:
R₉ₐ is methyl, ethyl, propyl, isopropyl, butyl, isobutyl, pentyl, isopentyl, acetyl, C(═O)—NH-phenyl, C(═O)-ethyl, C(═O)-cyclopropyl, CH₂-cyclobutyl, CH₂CH₂CH₂OH, CH₂CH₂CH₂OMe, or CH₂CH₂OH;
L_{c} is NH, NH—CH₂, NH—CH₂-(5-6 membered heteroarylene), C(═O)O—, C(═O)NH, or OC(═O)—NH; and
L_{d} is C₃₋₆ cycloalkyl, phenyl, or 5-10 memebred heteroaryl.

In another embodiment, the compound is a compound of formula C2:

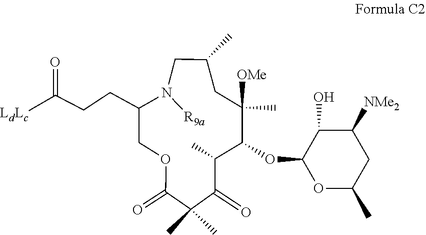

or a pharmaceutically acceptable salt thereof, wherein:
R₉ₐ is selected from the group consisting of H, C₁₋₆ alkyl, C₁₋₆ alkylene-OH, C₁₋₆ alkylene-O—C₁₋₆ alkyl, C(═O)C₁₋₆ alkyl, C₁₋₆ alkylene-cyclolkyl, C(═O)cycloalkyl, and C(═O)NH-aryl;
L_{c} is absent or is NH, or NH—C₁₋₄ alkylene; and
L_{d} is OH, C₃₋₆ cycloalkyl, 5-10 membered heterocycloalkyl, 6-10 membered aryl, or 5-10 memebred heteroaryl, wherein the cycloalkyl, heterocycloalkyl, aryl, and heteroaryl are each independently and optionally substituted with halo, C₁₋₄alkyl, C₁₋₄haloalkyl, or C₁₋₄alkoxy.

In a further embodiment of formula C₂:
R₉ₐ is methyl, ethyl, propyl, isopropyl, butyl, isobutyl, pentyl, isopentyl, acetyl, C(═O)—NH-phenyl, C(═O)-ethyl, C(═O)-cyclopropyl, CH₂-cyclobutyl, CH₂CH₂CH₂OH, CH₂CH₂CH₂OMe, or CH₂CH₂OH;
L_{c} is absent or is NH or NH—CH₂; and
L_{d} is OH, C₃₋₆ cycloalkyl, phenyl, 5-6 membered heteocycloalkyl, or 6-10 memebred nitrogen containing heteroaryl.

In another embodiment, R₁₀ₐ is selected from the group consisting of:

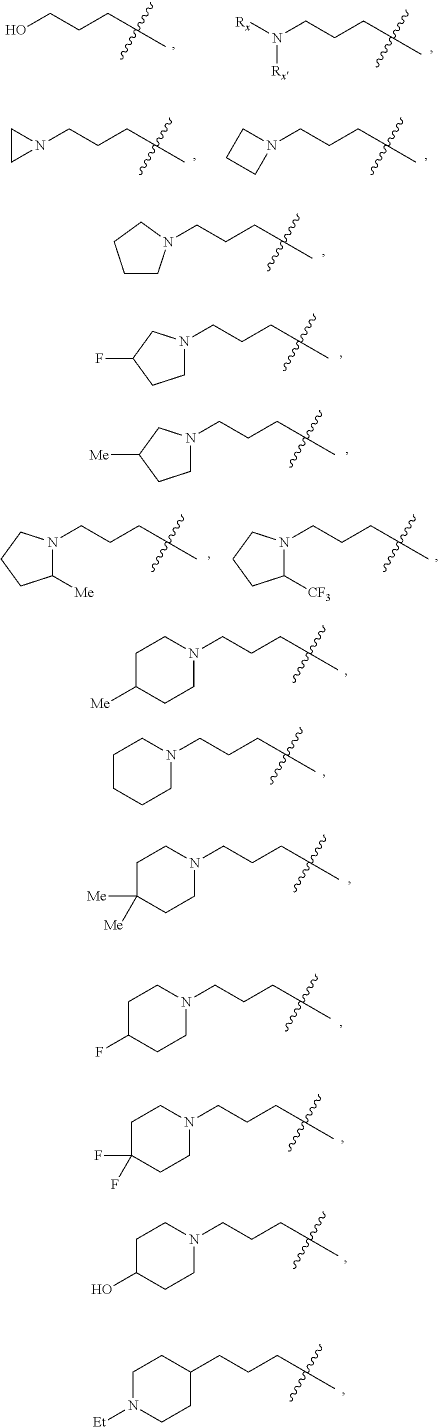

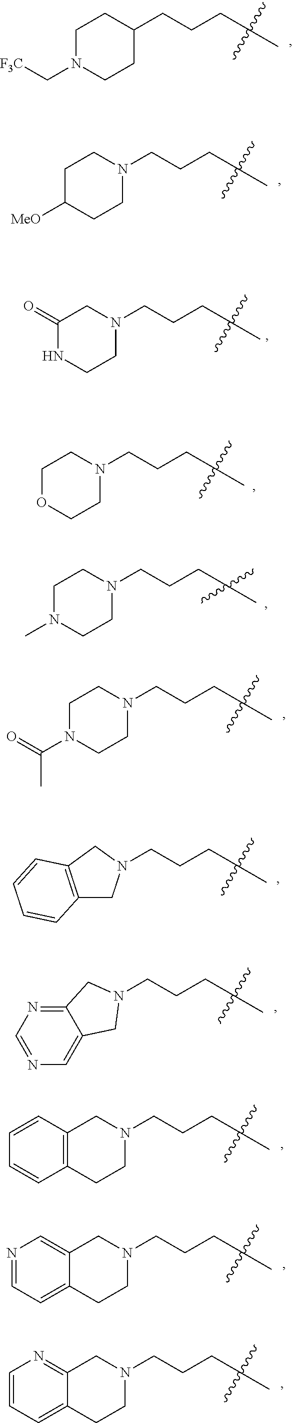

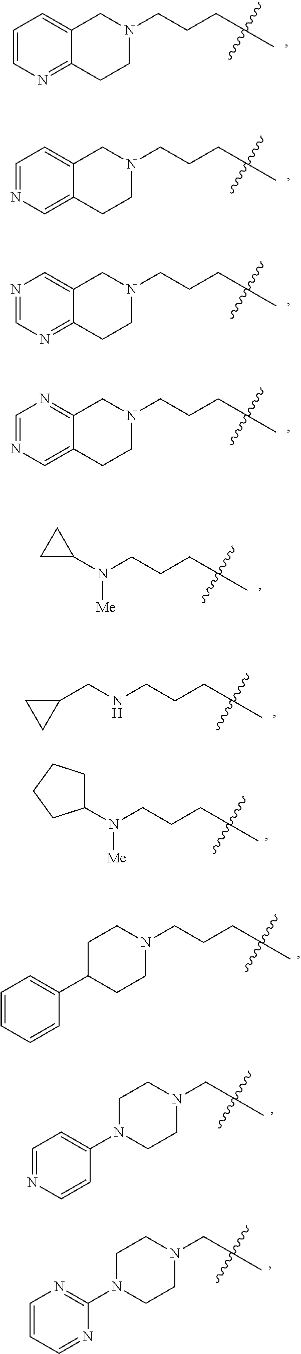

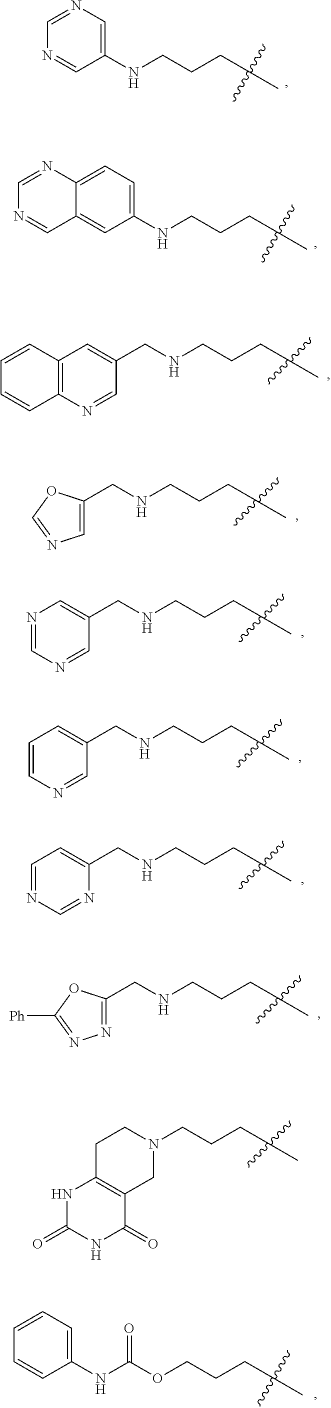

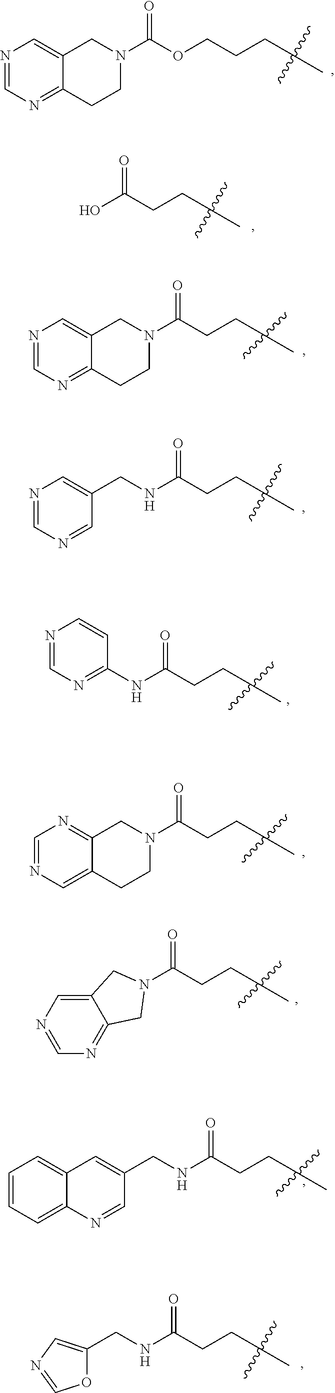

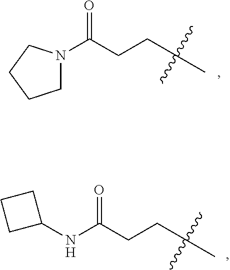

wherein
Rₓ and R_{y} are each independently H or C₁₋₄alkyl.

In some embodiments, R₁₀ₐ is

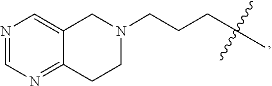 [SMILES: *CCCN1CCc2ncncc2C1]

In another embodiment, the compounds of formula I and formula II used in the method is selected from the compounds appearing in the following table or the pharmaceutically acceptable salts thereof.

TABLE A
Formula I and II Compounds

| Cmpd # | Structure |
|---|---|
| 1 | 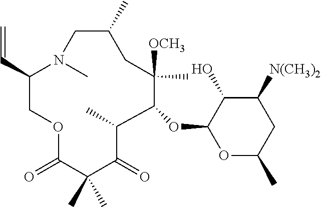 |
| 2 | 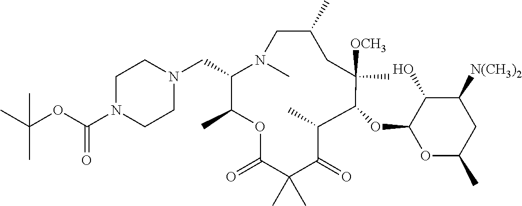 [SMILES: CO[C@]1(C)C[C@@H](C)CN(C)[C@@H](CN2CCN(C(=O)OC(C)(C)C)CC2)[C@H](C)OC(=O)C(C)(C)C(=O)[C@H](C)[C@H]1O[C@@H]1O[C@H](C)C[C@H](N(C)C)[C@H]1O] |
| 3 | 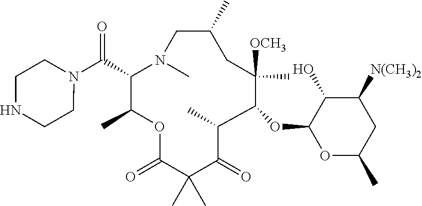 [SMILES: CO[C@]1(C)C[C@@H](C)CN(C)[C@@H](C(=O)N2CCNCC2)[C@H](C)OC(=O)C(C)(C)C(=O)[C@H](C)[C@H]1O[C@@H]1O[C@H](C)C[C@H](N(C)C)[C@H]1O] |
| 4 | 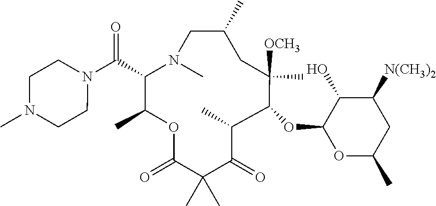 [SMILES: CO[C@]1(C)C[C@@H](C)CN(C)[C@@H](C(=O)N2CCN(C)CC2)[C@H](C)OC(=O)C(C)(C)C(=O)[C@H](C)[C@H]1O[C@@H]1O[C@H](C)C[C@H](N(C)C)[C@H]1O] |
| 5 | 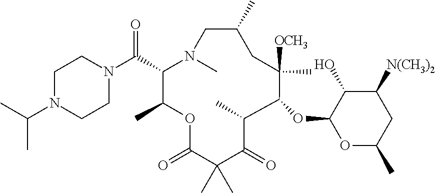 [SMILES: CO[C@]1(C)C[C@@H](C)CN(C)[C@@H](C(=O)N2CCN(C(C)C)CC2)[C@H](C)OC(=O)C(C)(C)C(=O)[C@H](C)[C@H]1O[C@@H]1O[C@H](C)C[C@H](N(C)C)[C@H]1O] |
| 6 | 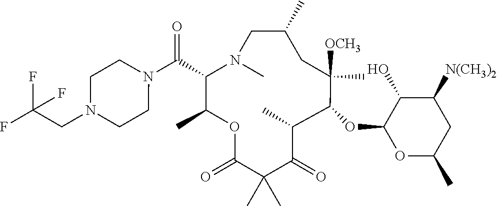 [SMILES: CO[C@]1(C)C[C@@H](C)CN(C)[C@@H](C(=O)N2CCN(CC(F)(F)F)CC2)[C@H](C)OC(=O)C(C)(C)C(=O)[C@H](C)[C@H]1O[C@@H]1O[C@H](C)C[C@H](N(C)C)[C@H]1O] |
| 7 | 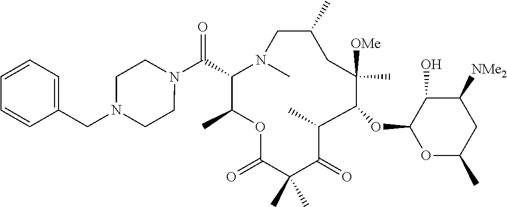 |
| 8 | 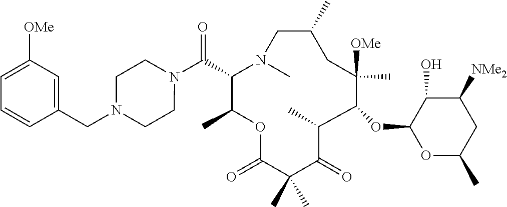 |
| 9 | 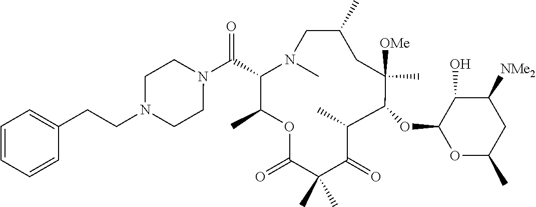 |
| 10 | 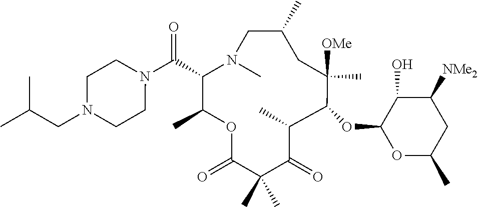 |
| 11 | 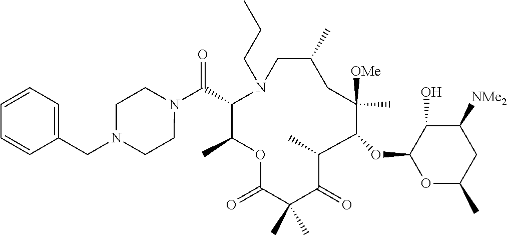 |
| 12 | 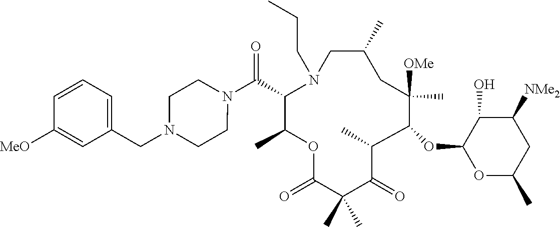 |
| 13 | 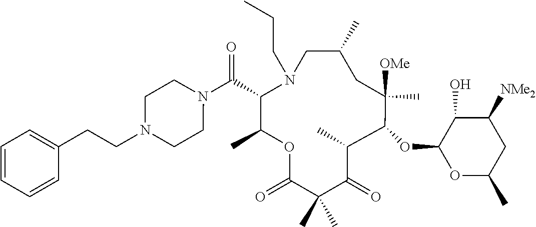 |
| 14 | 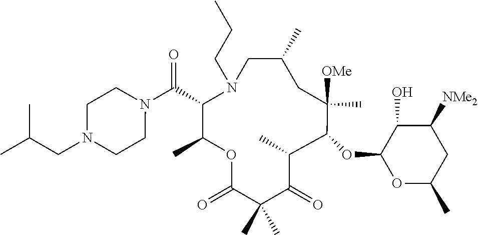 |
| 15 | 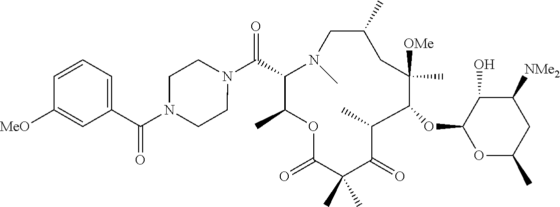 |
| 16 | 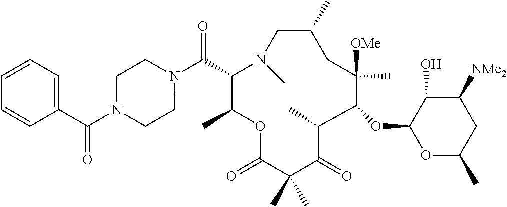 |
| 17 | 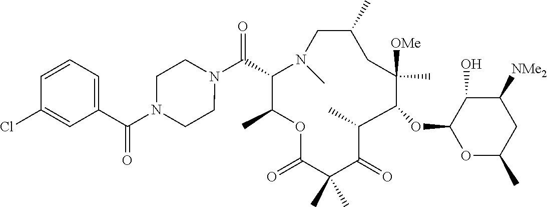 |
| 18 | 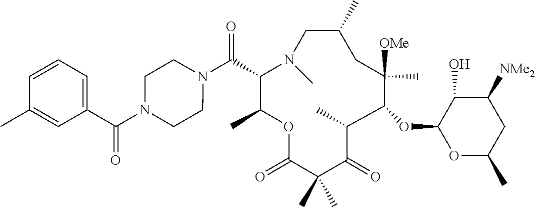 |
| 19 | 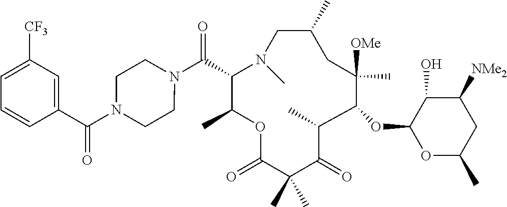 |
| 20 | 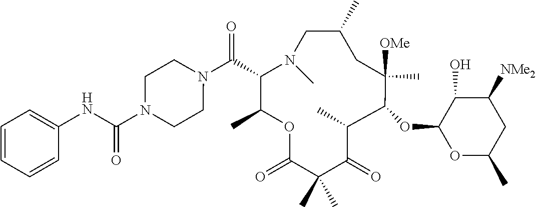 |
| 21 | 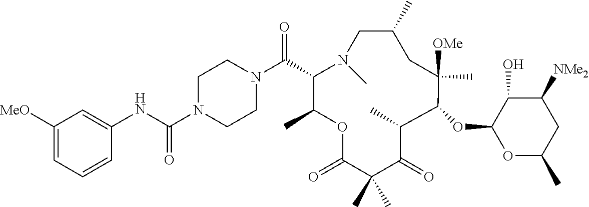 |
| 22 | 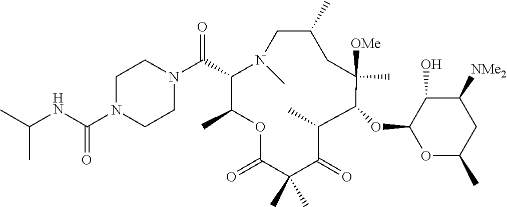 |
| 23 | 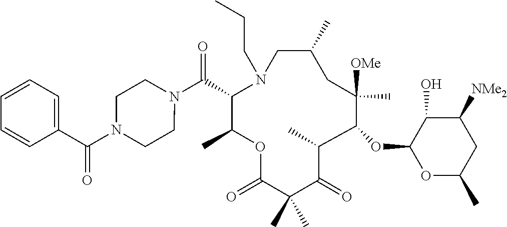 |
| 24 | 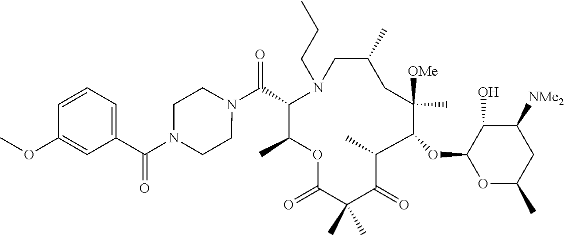 |
| 25 | 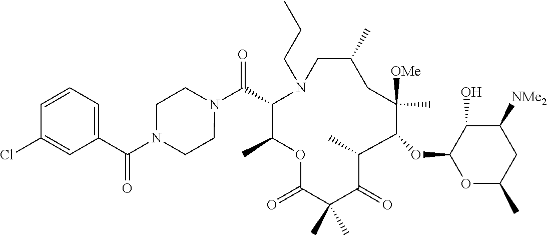 |
| 26 | 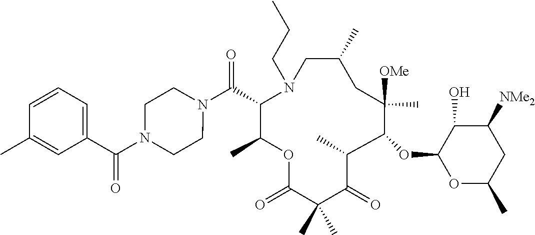 |
| 27 | 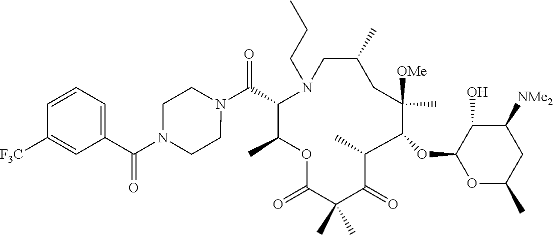 |
| 28 | 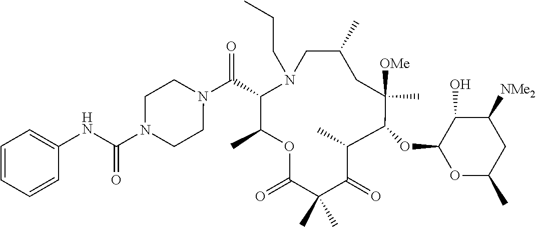 |
| 29 | 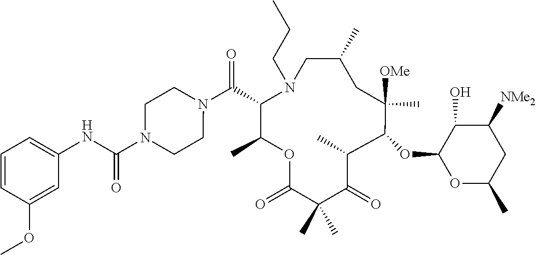 |
| 30 | 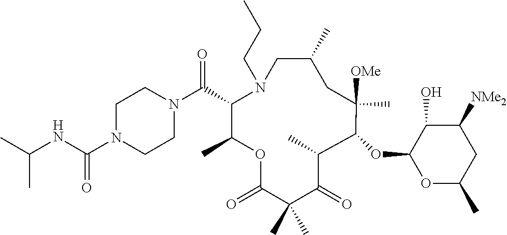 |
| 31 | 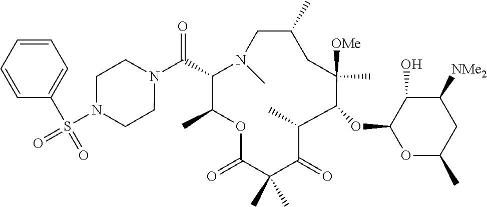 |
| 32 | 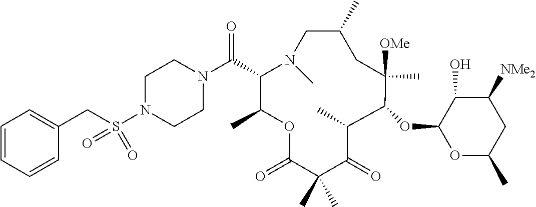 |
| 33 | 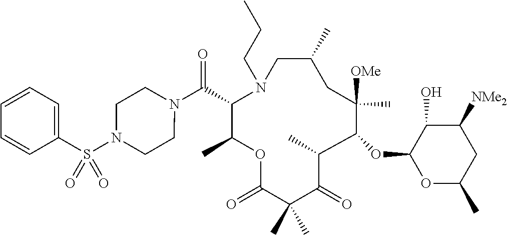 |
| 34 | 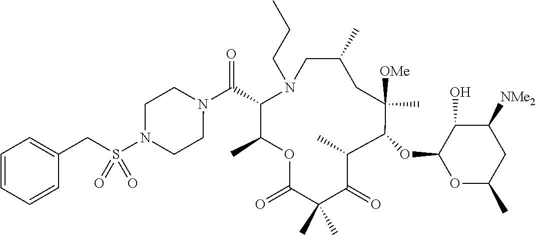 |
| 35 | 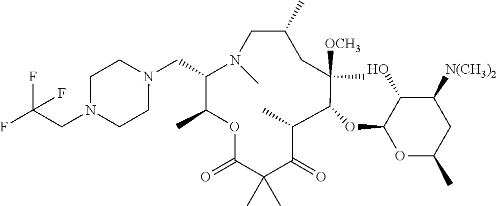 [SMILES: CO[C@]1(C)C[C@@H](C)CN(C)[C@@H](CN2CCN(CC(F)(F)F)CC2)[C@H](C)OC(=O)C(C)(C)C(=O)[C@H](C)[C@H]1O[C@@H]1O[C@H](C)C[C@H](N(C)C)[C@H]1O] |
| 36 | 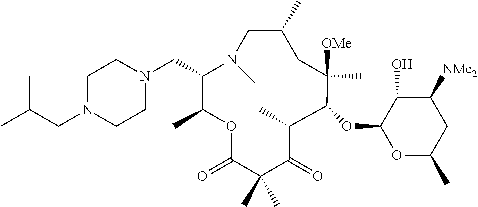 |
| 37 | 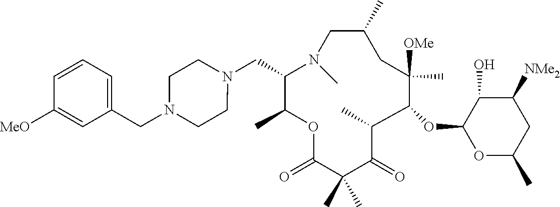 |
| 38 | 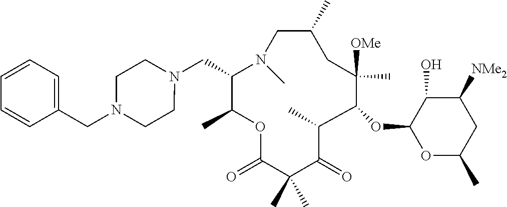 |
| 39 | 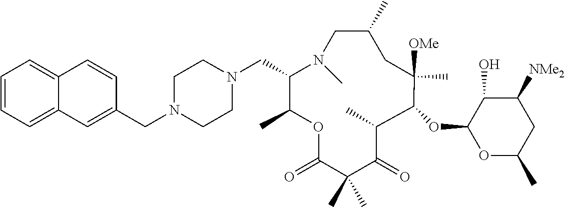 |
| 40 | 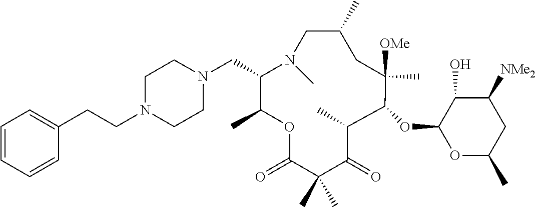 |
| 41 | 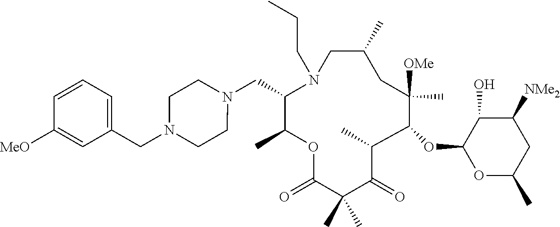 |
| 42 | 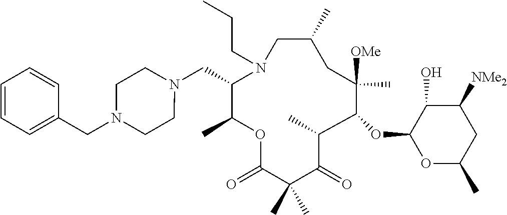 |
| 43 | 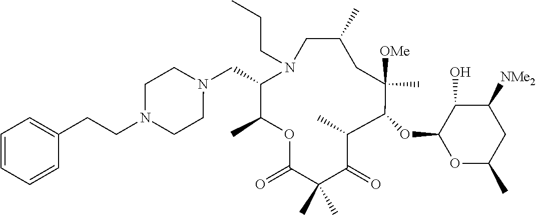 |
| 44 | 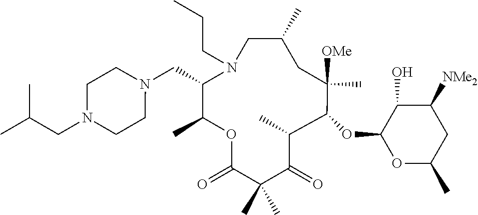 |
| 45 | 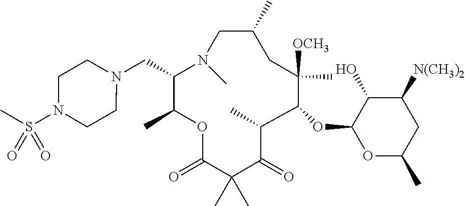 [SMILES: CO[C@]1(C)C[C@@H](C)CN(C)[C@@H](CN2CCN(S(C)(=O)=O)CC2)[C@H](C)OC(=O)C(C)(C)C(=O)[C@H](C)[C@H]1O[C@@H]1O[C@H](C)C[C@H](N(C)C)[C@H]1O] |
| 46 | 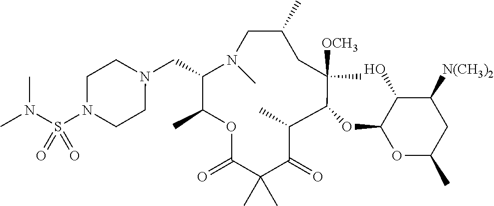 [SMILES: CO[C@]1(C)C[C@@H](C)CN(C)[C@@H](CN2CCN(S(=O)(=O)N(C)C)CC2)[C@H](C)OC(=O)C(C)(C)C(=O)[C@H](C)[C@H]1O[C@@H]1O[C@H](C)C[C@H](N(C)C)[C@H]1O] |
| 47 | 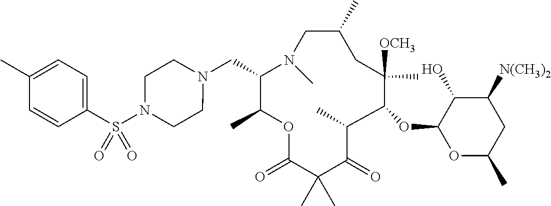 [SMILES: CO[C@]1(C)C[C@@H](C)CN(C)[C@@H](CN2CCN(S(=O)(=O)c3ccc(C)cc3)CC2)[C@H](C)OC(=O)C(C)(C)C(=O)[C@H](C)[C@H]1O[C@@H]1O[C@H](C)C[C@H](N(C)C)[C@H]1O] |
| 48 | 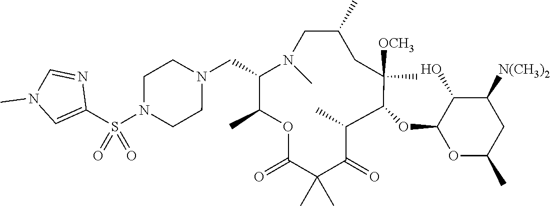 [SMILES: CO[C@]1(C)C[C@@H](C)CN(C)[C@@H](CN2CCN(S(=O)(=O)c3cn(C)cn3)CC2)[C@H](C)OC(=O)C(C)(C)C(=O)[C@H](C)[C@H]1O[C@@H]1O[C@H](C)C[C@H](N(C)C)[C@H]1O] |
| 49 | 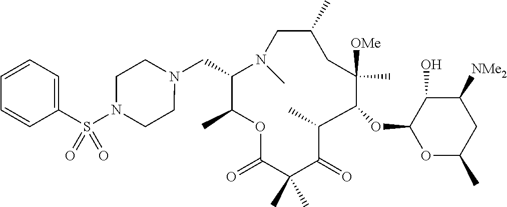 |
| 50 | 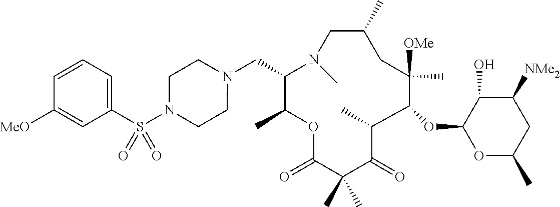 |
| 51 | 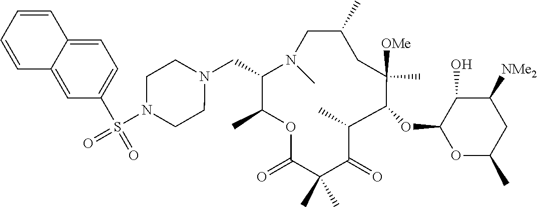 |
| 52 | 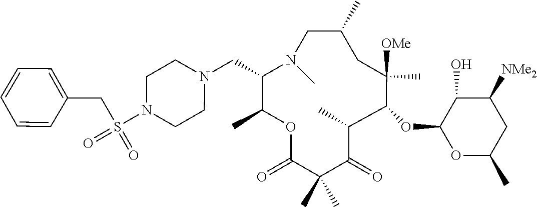 |
| 53 | 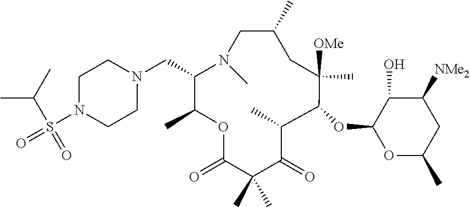 |
| 54 | 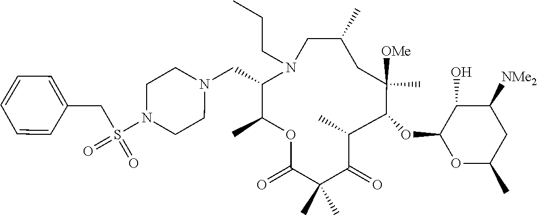 |
| 55 | 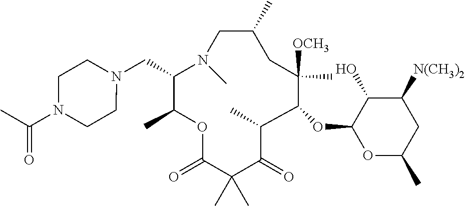 [SMILES: CO[C@]1(C)C[C@@H](C)CN(C)[C@@H](CN2CCN(C(C)=O)CC2)[C@H](C)OC(=O)C(C)(C)C(=O)[C@H](C)[C@H]1O[C@@H]1O[C@H](C)C[C@H](N(C)C)[C@H]1O] |
| 56 | 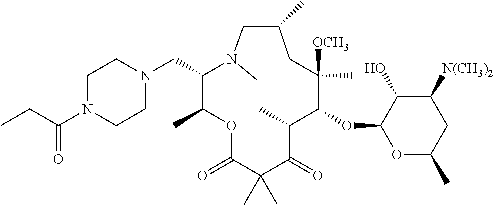 [SMILES: CCC(=O)N1CCN(C[C@H]2[C@H](C)OC(=O)C(C)(C)C(=O)[C@H](C)[C@@H](O[C@@H]3O[C@H](C)C[C@H](N(C)C)[C@H]3O)[C@](C)(OC)C[C@@H](C)CN2C)CC1] |
| 57 | 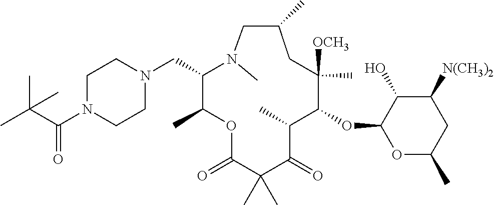 [SMILES: CO[C@]1(C)C[C@@H](C)CN(C)[C@@H](CN2CCN(C(=O)C(C)(C)C)CC2)[C@H](C)OC(=O)C(C)(C)C(=O)[C@H](C)[C@H]1O[C@@H]1O[C@H](C)C[C@H](N(C)C)[C@H]1O] |
| 59 | 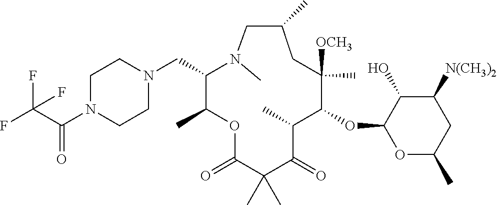 [SMILES: CO[C@]1(C)C[C@@H](C)CN(C)[C@@H](CN2CCN(C(=O)C(F)(F)F)CC2)[C@H](C)OC(=O)C(C)(C)C(=O)[C@H](C)[C@H]1O[C@@H]1O[C@H](C)C[C@H](N(C)C)[C@H]1O] |
| 60 | 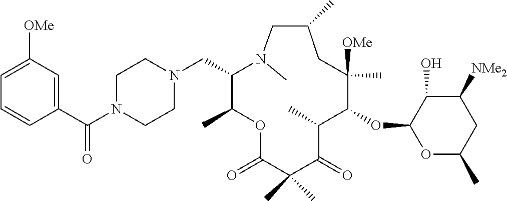 |
| 61 | 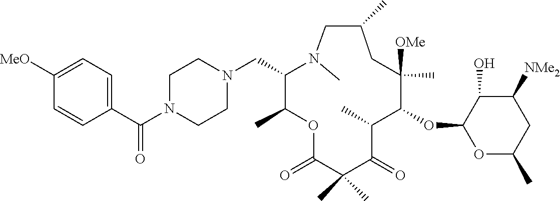 |
| 62 | 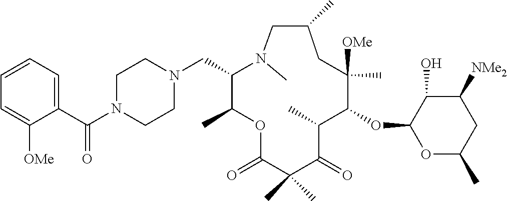 |
| 63 | 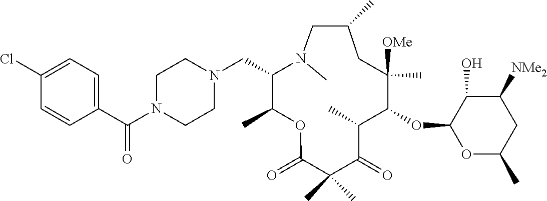 |
| 64 | 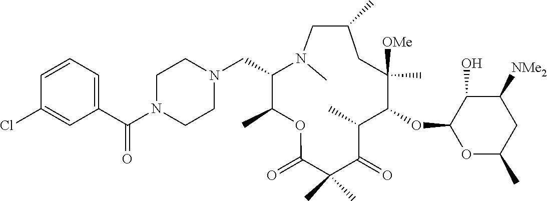 |
| 65 | 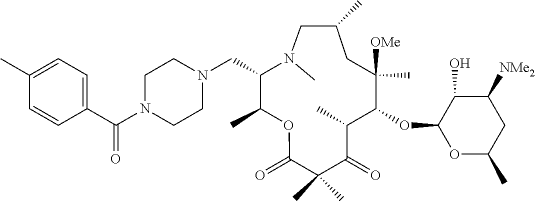 |
| 66 | 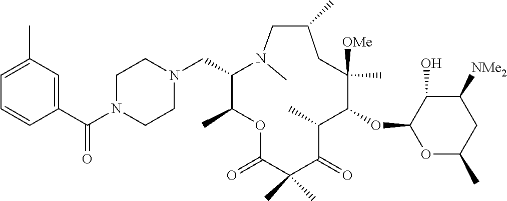 |
| 67 | 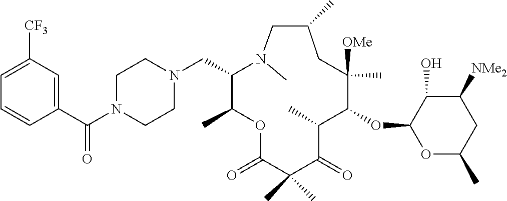 |
| 68 | 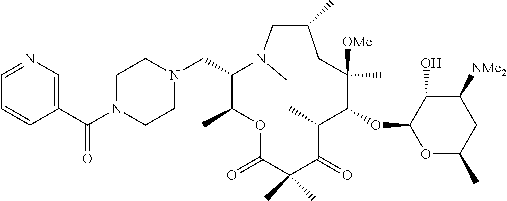 |
| 69 | 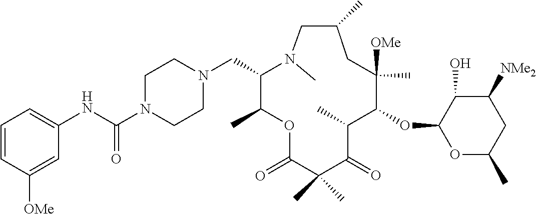 |
| 70 | 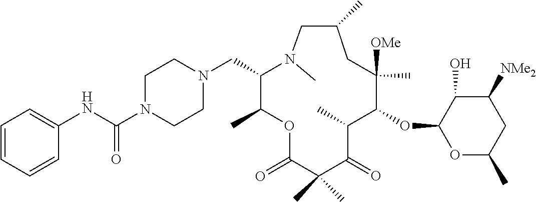 |
| 71 | 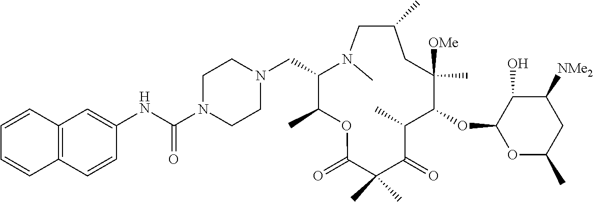 |
| 72 | 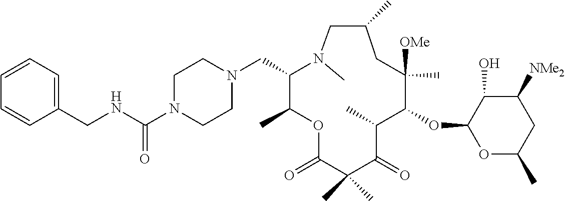 |
| 73 | 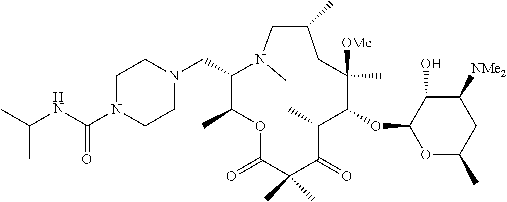 |
| 74 | 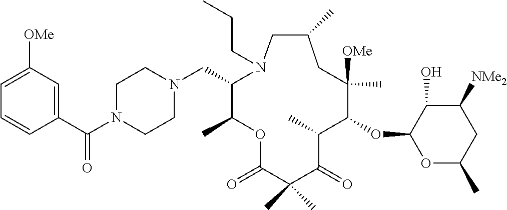 |
| 75 | 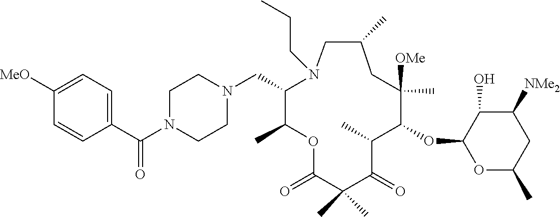 |
| 76 | 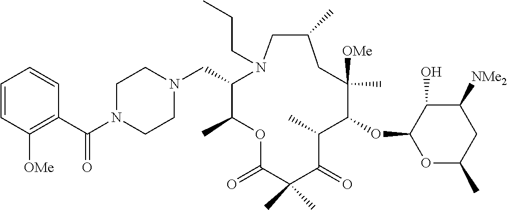 |
| 77 | 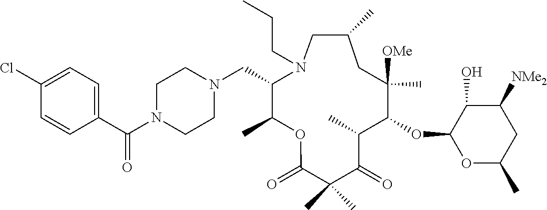 |
| 78 | 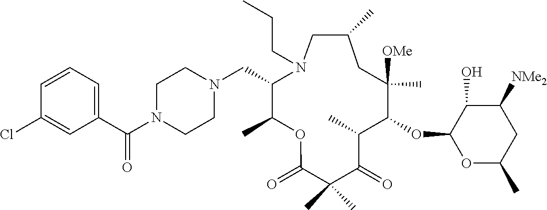 |
| 79 | 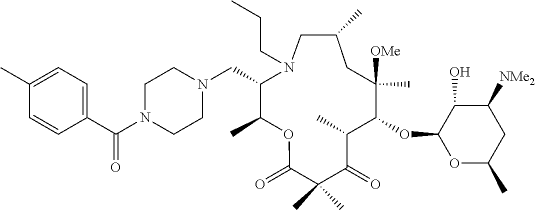 |
| 80 | 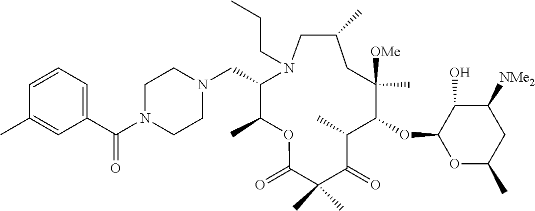 |
| 81 | 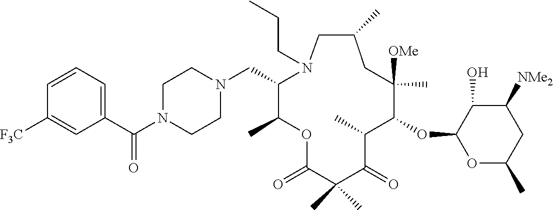 |
| 82 | 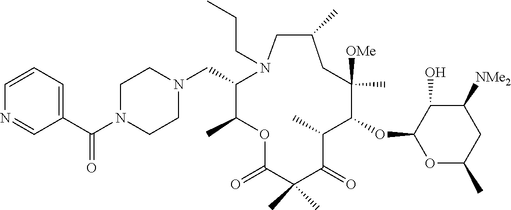 |
| 83 | 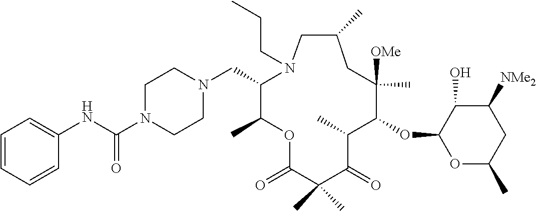 |
| 84 | 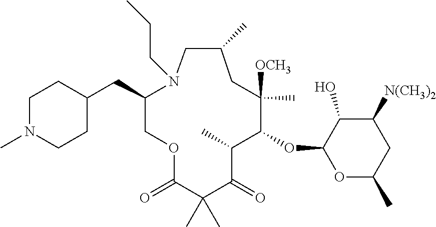 [SMILES: CCCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@H]1CC1CCN(C)CC1] |
| 85 | 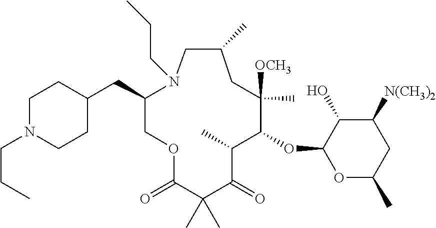 [SMILES: CCCN1CCC(C[C@@H]2COC(=O)C(C)(C)C(=O)[C@H](C)[C@@H](O[C@@H]3O[C@H](C)C[C@H](N(C)C)[C@H]3O)[C@](C)(OC)C[C@@H](C)CN2CCC)CC1] |
| 86 | 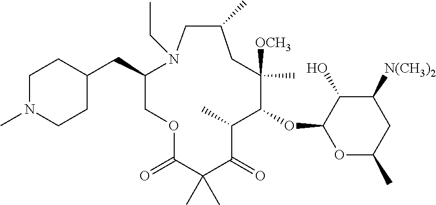 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@H]1CC1CCN(C)CC1] |
| 87 | 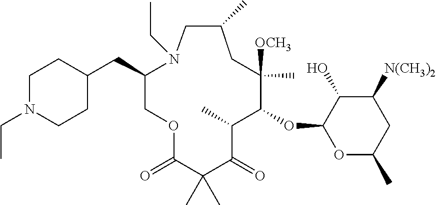 [SMILES: CCN1CCC(C[C@@H]2COC(=O)C(C)(C)C(=O)[C@H](C)[C@@H](O[C@@H]3O[C@H](C)C[C@H](N(C)C)[C@H]3O)[C@](C)(OC)C[C@@H](C)CN2CC)CC1] |
| 88 | 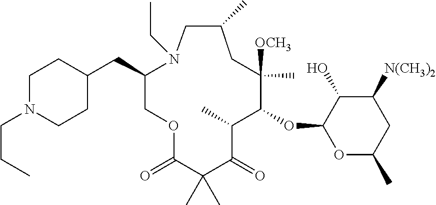 [SMILES: CCCN1CCC(C[C@@H]2COC(=O)C(C)(C)C(=O)[C@H](C)[C@@H](O[C@@H]3O[C@H](C)C[C@H](N(C)C)[C@H]3O)[C@](C)(OC)C[C@@H](C)CN2CC)CC1] |
| 89 | 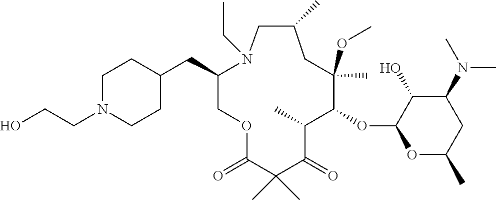 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@H]1CC1CCN(CCO)CC1] |
| 90 | 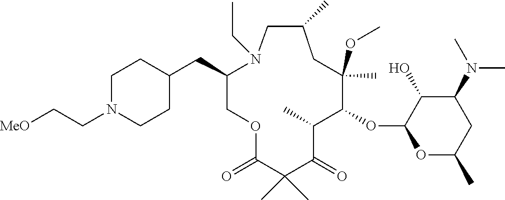 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@H]1CC1CCN(CCOC)CC1] |
| 91 | 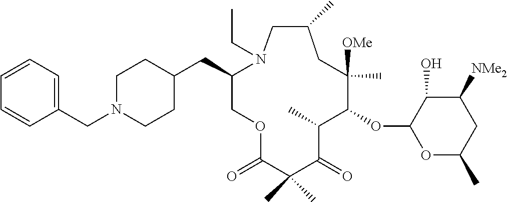 |
| 92 | 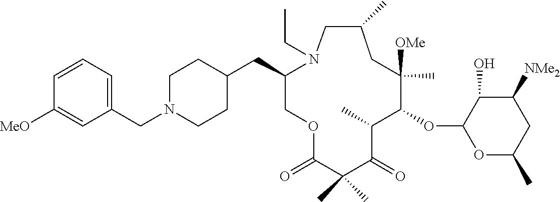 |
| 93 | 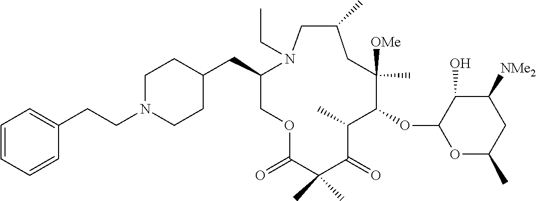 |
| 94 | 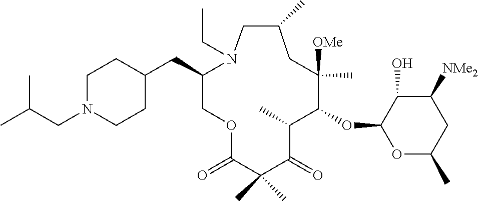 |
| 95 | 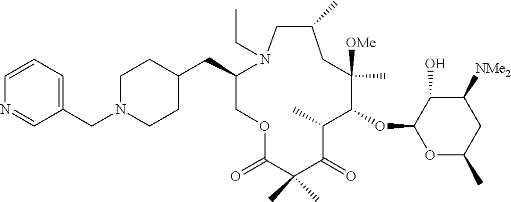 |
| 96 | 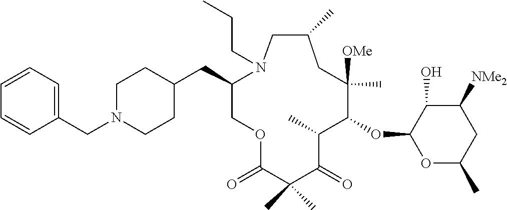 |
| 97 | 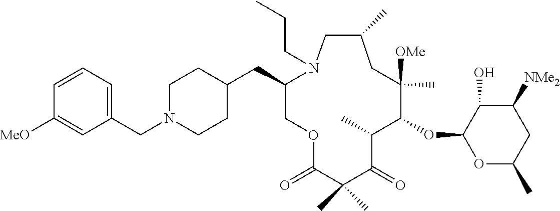 |
| 98 | 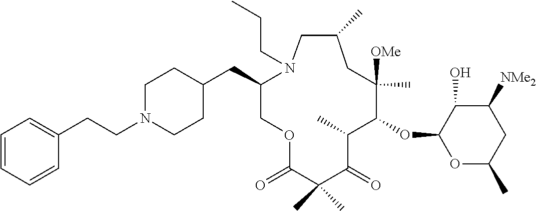 |
| 99 | 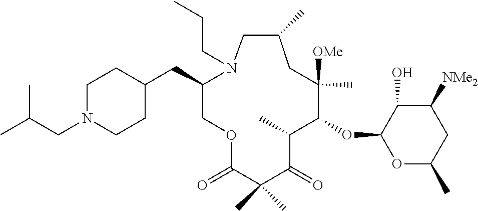 |
| 100 | 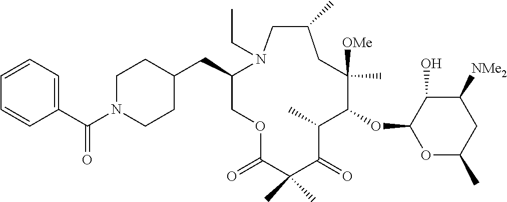 |
| 101 | 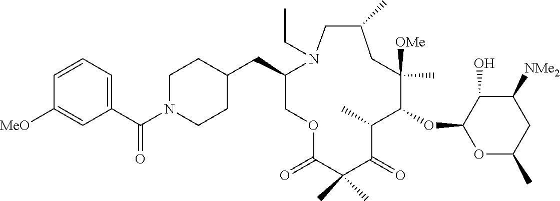 |
| 102 | 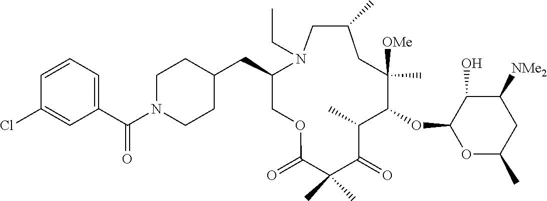 |
| 103 | 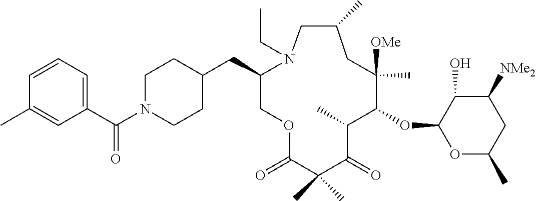 |
| 104 | 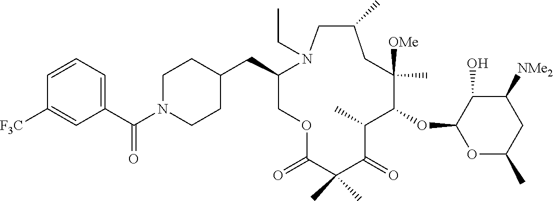 |
| 105 | 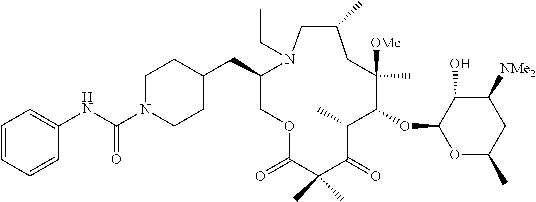 |
| 106 | 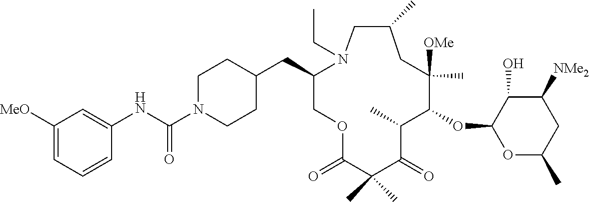 |
| 107 | 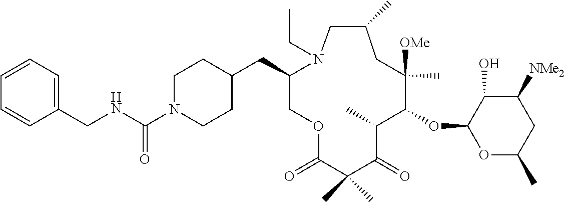 |
| 108 | 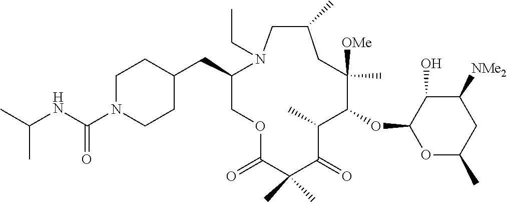 |
| 109 | 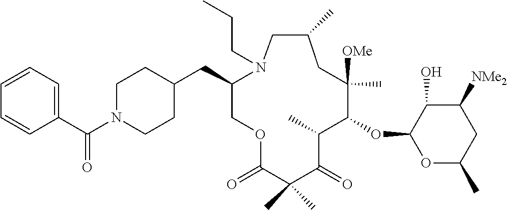 |
| 110 | 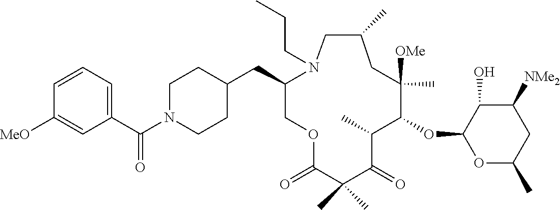 |
| 111 | 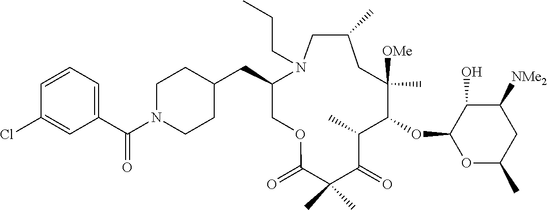 |
| 112 | 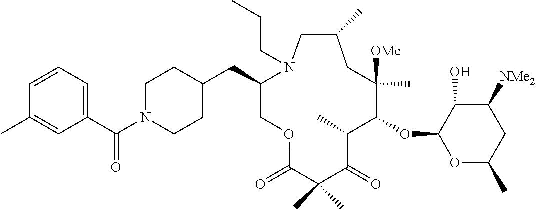 |
| 113 | 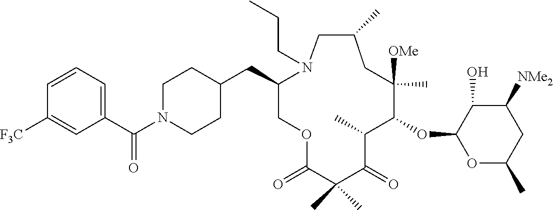 |
| 114 | 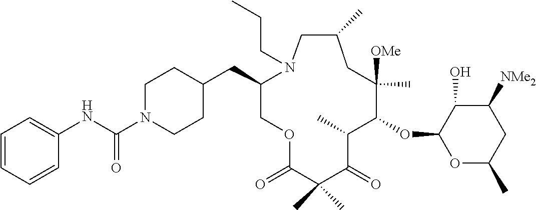 |
| 115 | 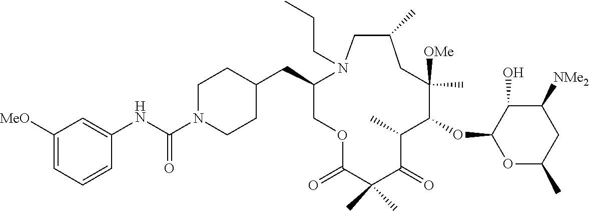 |
| 116 | 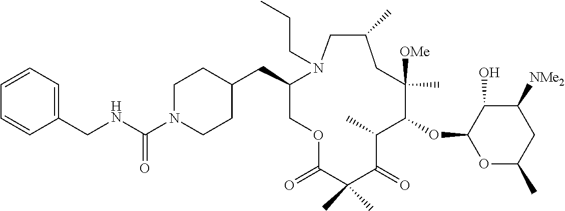 |
| 117 | 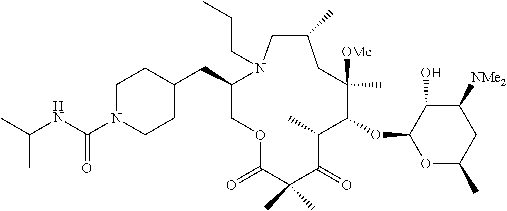 |
| 118 | 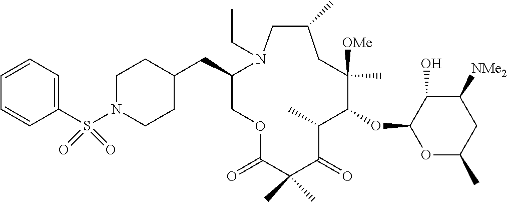 |
| 119 | 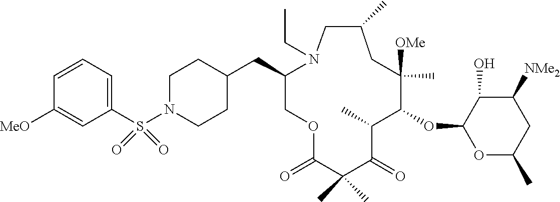 |
| 120 | 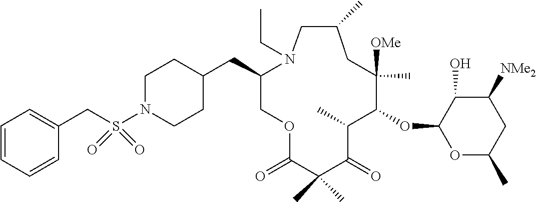 |
| 121 | 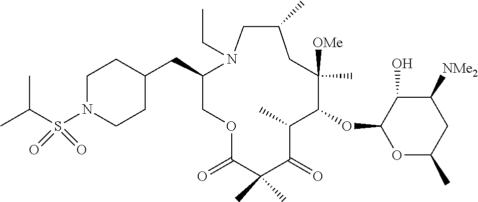 |
| 122 | 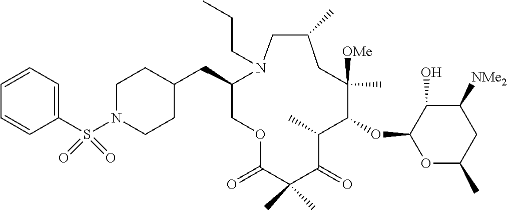 |
| 123 | 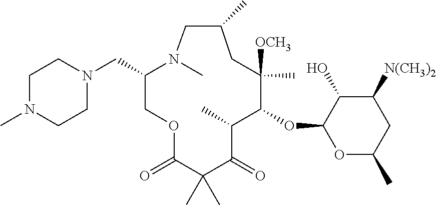 [SMILES: CO[C@]1(C)C[C@@H](C)CN(C)[C@@H](CN2CCN(C)CC2)COC(=O)C(C)(C)C(=O)[C@H](C)[C@H]1O[C@@H]1O[C@H](C)C[C@H](N(C)C)[C@H]1O] |
| 124 | 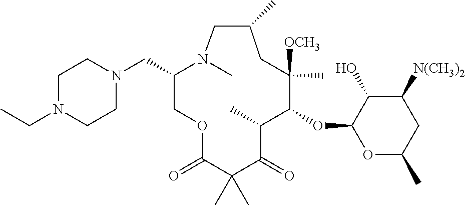 [SMILES: CCN1CCN(C[C@H]2COC(=O)C(C)(C)C(=O)[C@H](C)[C@@H](O[C@@H]3O[C@H](C)C[C@H](N(C)C)[C@H]3O)[C@](C)(OC)C[C@@H](C)CN2C)CC1] |
| 125 | 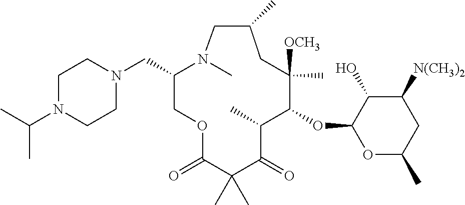 [SMILES: CO[C@]1(C)C[C@@H](C)CN(C)[C@@H](CN2CCN(C(C)C)CC2)COC(=O)C(C)(C)C(=O)[C@H](C)[C@H]1O[C@@H]1O[C@H](C)C[C@H](N(C)C)[C@H]1O] |
| 126 | 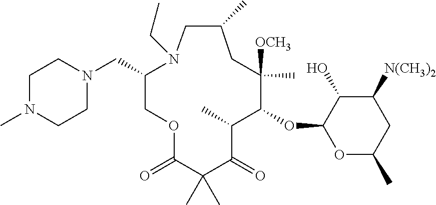 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CN1CCN(C)CC1] |
| 127 | 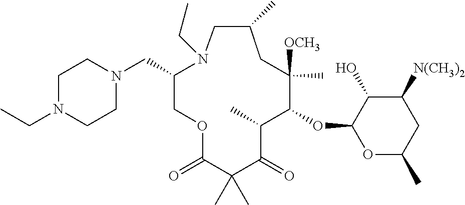 [SMILES: CCN1CCN(C[C@H]2COC(=O)C(C)(C)C(=O)[C@H](C)[C@@H](O[C@@H]3O[C@H](C)C[C@H](N(C)C)[C@H]3O)[C@](C)(OC)C[C@@H](C)CN2CC)CC1] |
| 128 | 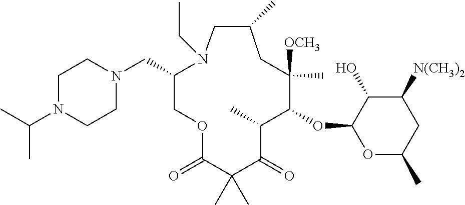 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CN1CCN(C(C)C)CC1] |
| 129 | 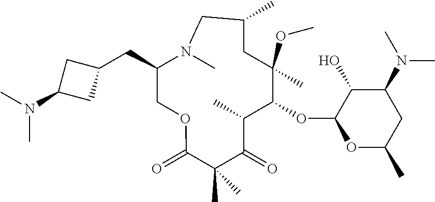 |
| 130 | 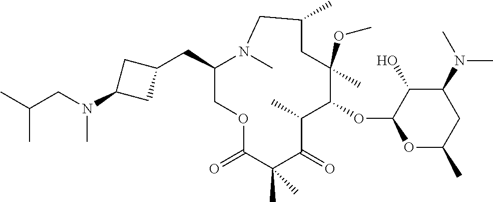 |
| 131 | 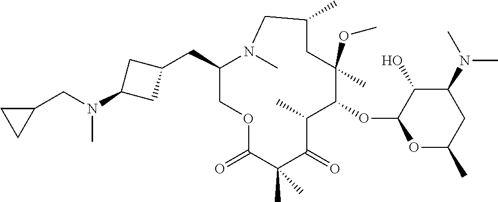 |
| 132 | 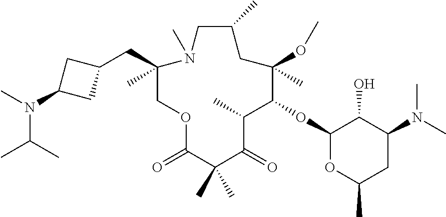 |
| 133 | 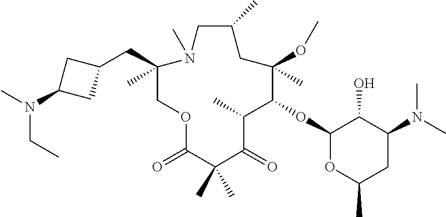 |
| 134 | 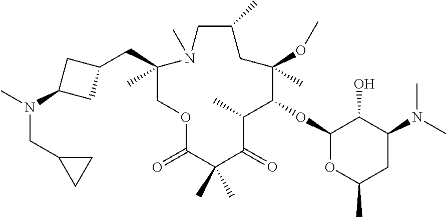 |
| 135 | 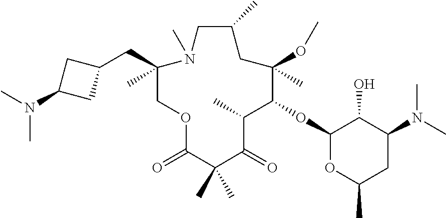 |
| 136 | 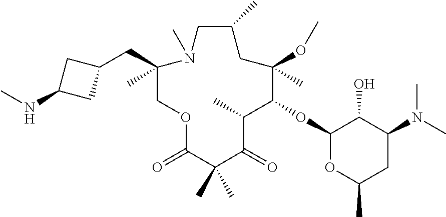 |
| 137 | 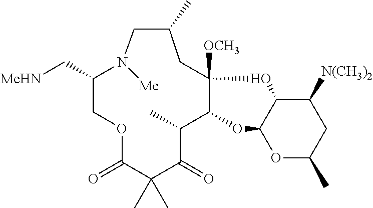 [SMILES: CNC[C@H]1COC(=O)C(C)(C)C(=O)[C@H](C)[C@H]2O[C@@H]3O[C@H](C)C[C@H](N(C)C)[C@H]3O[C@@]2(OC)C[C@@H](C)CN1C] |
| 138 | 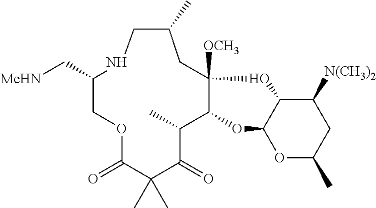 [SMILES: CNC[C@H]1COC(=O)C(C)(C)C(=O)[C@H](C)[C@H]2O[C@@H]3O[C@H](C)C[C@H](N(C)C)[C@H]3O[C@@]2(OC)C[C@@H](C)CN1] |
| 139 | 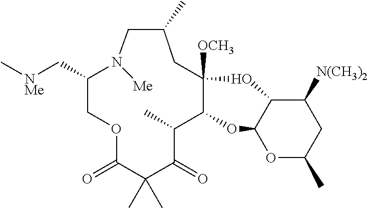 [SMILES: CO[C@]12C[C@@H](C)CN(C)[C@@H](CN(C)C)COC(=O)C(C)(C)C(=O)[C@H](C)[C@H]1O[C@@H]1O[C@H](C)C[C@H](N(C)C)[C@H]1O2] |
| 140 | 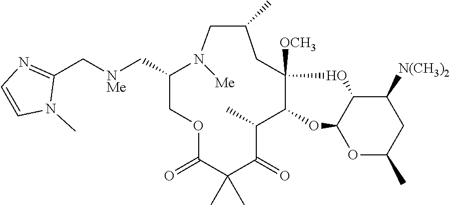 [SMILES: CO[C@]12C[C@@H](C)CN(C)[C@@H](CN(C)Cc3nccn3C)COC(=O)C(C)(C)C(=O)[C@H](C)[C@H]1O[C@@H]1O[C@H](C)C[C@H](N(C)C)[C@H]1O2] |
| 141 | 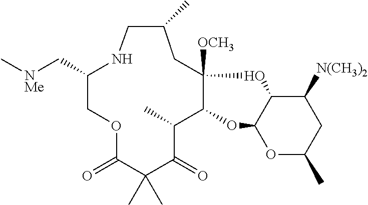 [SMILES: CO[C@]12C[C@@H](C)CN[C@@H](CN(C)C)COC(=O)C(C)(C)C(=O)[C@H](C)[C@H]1O[C@@H]1O[C@H](C)C[C@H](N(C)C)[C@H]1O2] |
| 142 | 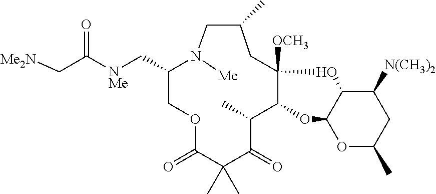 [SMILES: CO[C@]12C[C@@H](C)CN(C)[C@@H](CN(C)C(=O)CN(C)C)COC(=O)C(C)(C)C(=O)[C@H](C)[C@H]1O[C@@H]1O[C@H](C)C[C@H](N(C)C)[C@H]1O2] |
| 143 | 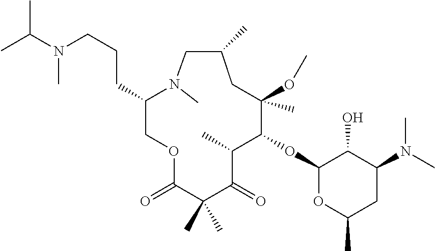 |
| 144 | 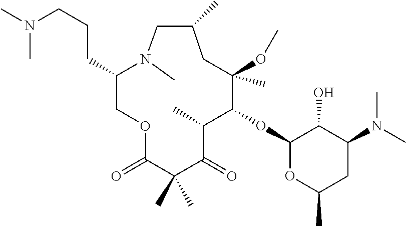 |
| 145 | 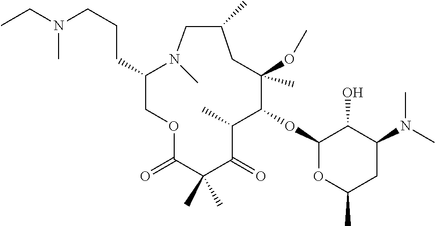 |
| 146 | 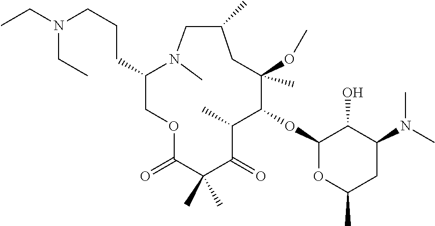 |
| 147 | 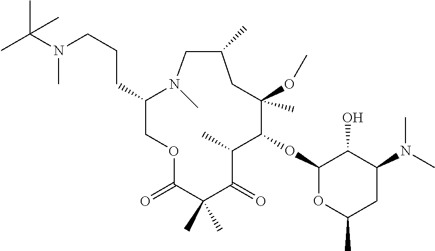 |
| 148 | 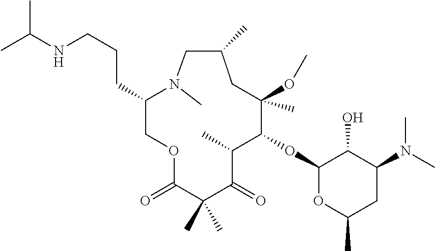 |
| 149 | 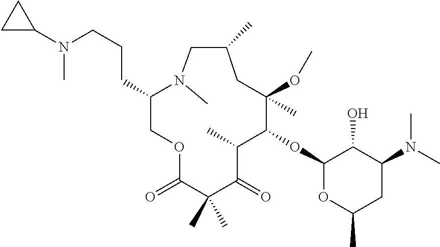 |
| 150 | 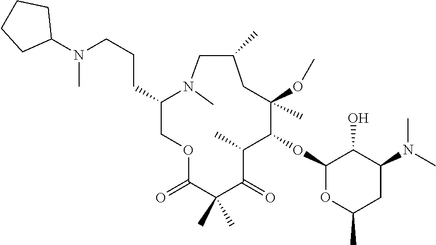 |
| 151 | 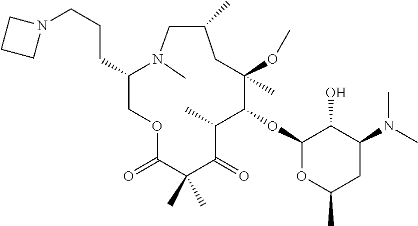 |
| 152 | 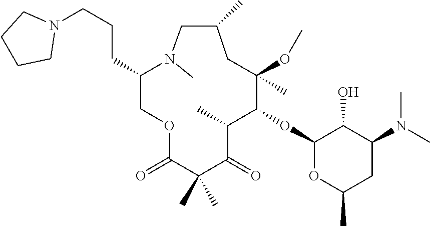 |
| 153 | 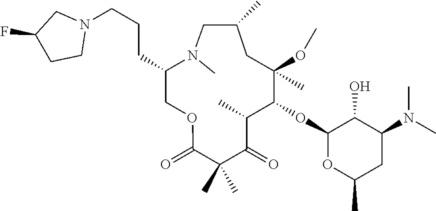 |
| 154 | 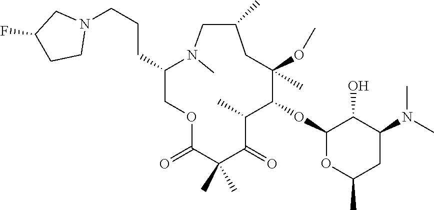 |
| 155 | 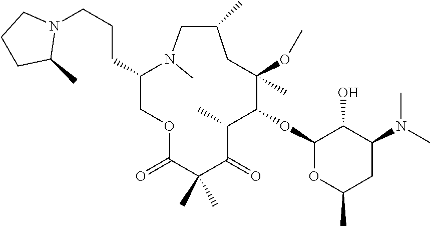 |
| 156 | 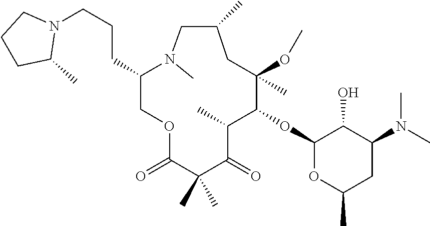 |
| 157 | 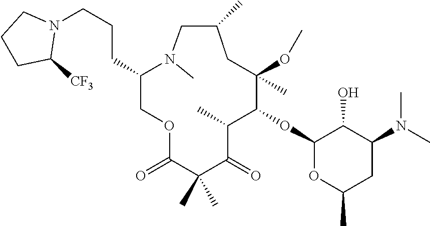 |
| 158 | 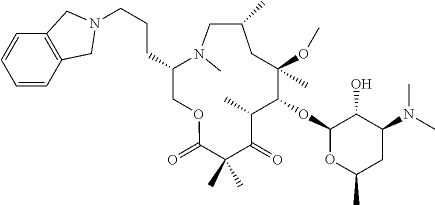 |
| 159 | 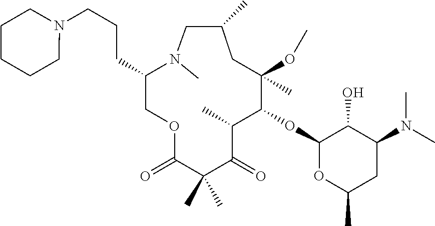 |
| 160 | 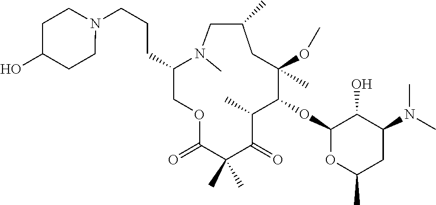 |
| 161 | 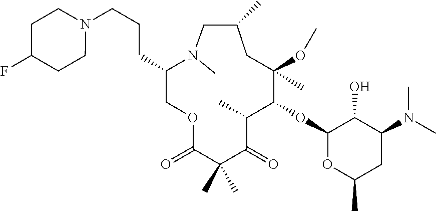 |
| 162 | 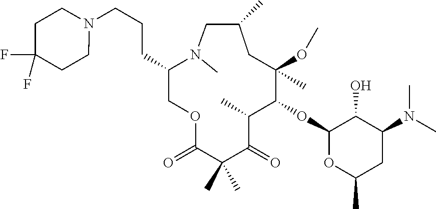 |
| 163 | 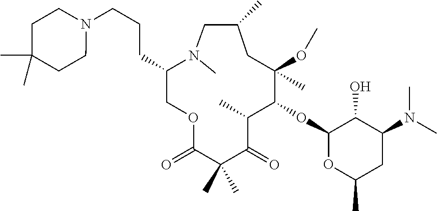 |
| 164 | 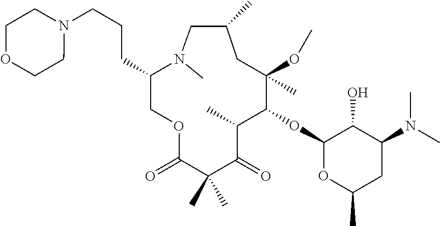 |
| 165 | 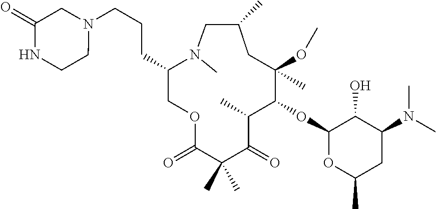 |
| 166 | 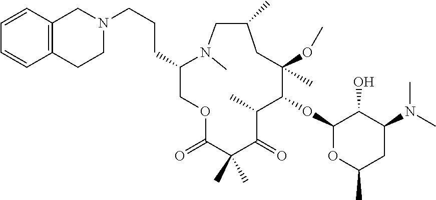 |
| 167 | 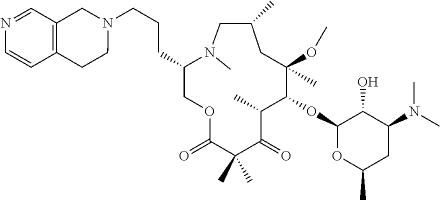 |
| 168 | 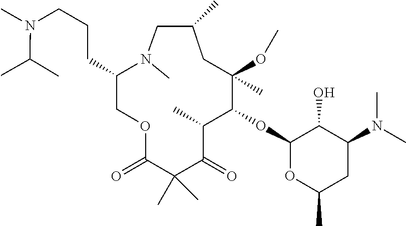 [SMILES: CO[C@]1(C)C[C@@H](C)CN(C)[C@@H](CCCN(C)C(C)C)COC(=O)C(C)(C)C(=O)[C@H](C)[C@H]1O[C@@H]1O[C@H](C)C[C@H](N(C)C)[C@H]1O] |
| 169 | 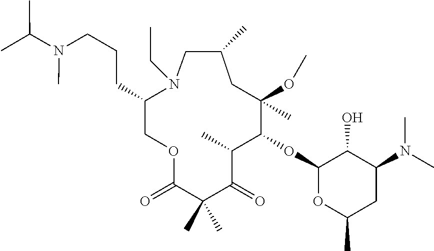 |
| 170 | 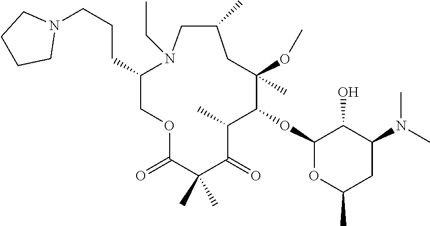 |
| 171 | 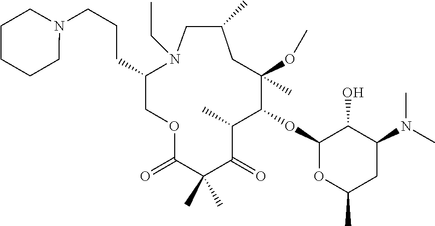 |
| 172 | 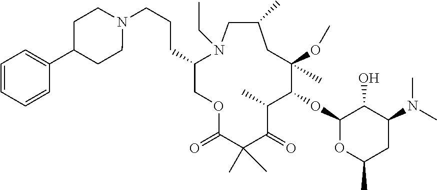 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCC(c2ccccc2)CC1] |
| 173 | 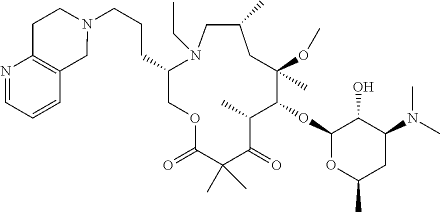 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCc2ncccc2C1] |
| 174 | 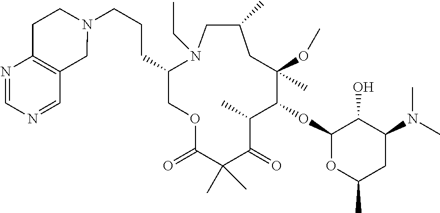 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCc2ncncc2C1] |
| 175 | 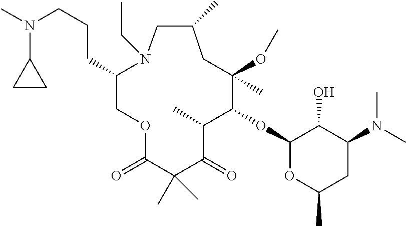 |
| 176 | 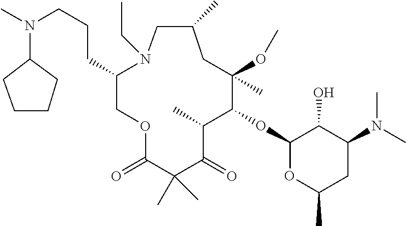 |
| 177 | 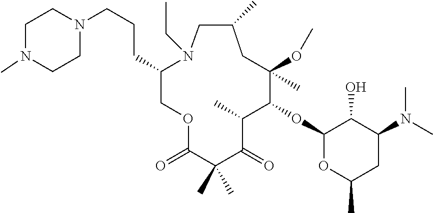 |
| 178 | 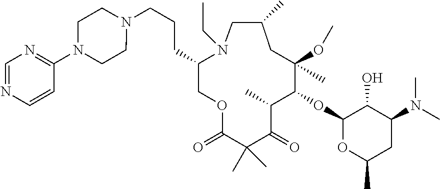 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCN(c2ccncn2)CC1] |
| 179 | 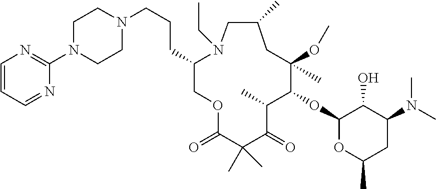 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCN(c2ncccn2)CC1] |
| 180 | 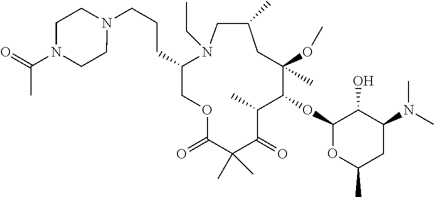 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCN(C(C)=O)CC1] |
| 181 | 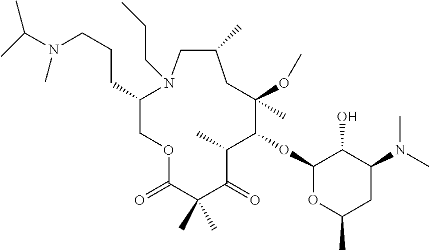 |
| 182 | 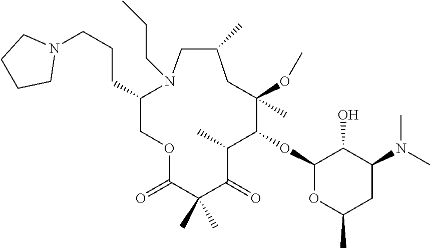 |
| 183 | 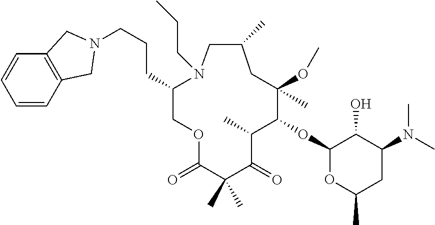 |
| 184 | 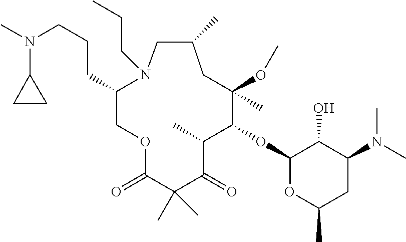 |
| 185 | 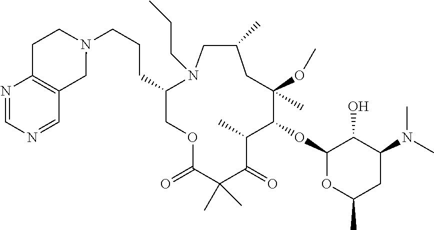 [SMILES: CCCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCc2ncncc2C1] |
| 186 | 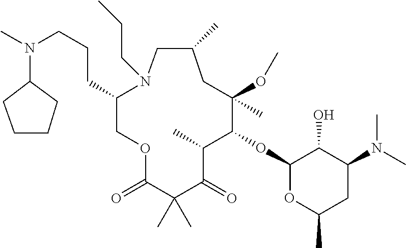 |
| 187 | 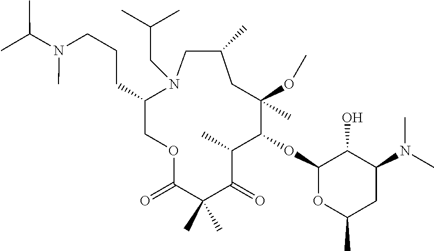 |
| 188 | 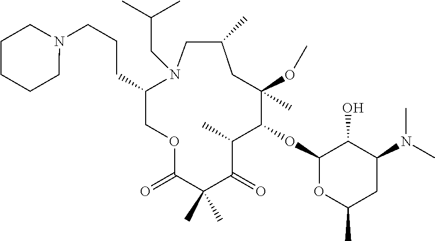 |
| 189 | 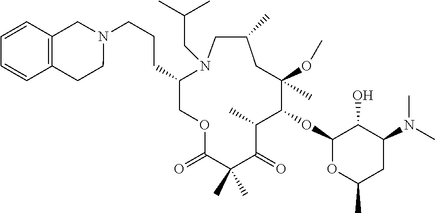 |
| 191 | 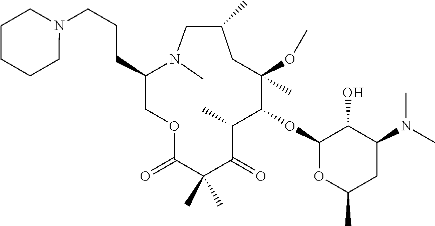 |
| 192 | 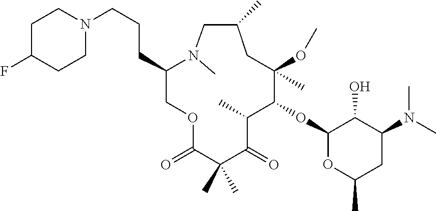 |
| 193 | 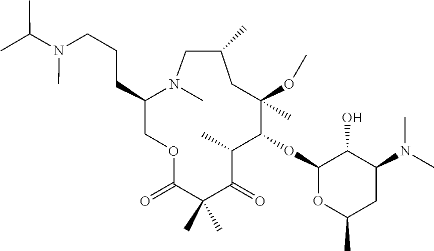 |
| 194 | 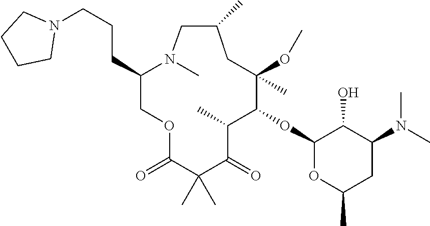 [SMILES: CO[C@]1(C)C[C@@H](C)CN(C)[C@H](CCCN2CCCC2)COC(=O)C(C)(C)C(=O)[C@H](C)[C@H]1O[C@@H]1O[C@H](C)C[C@H](N(C)C)[C@H]1O] |
| 195 | 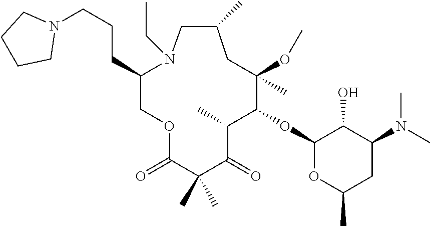 |
| 196 | 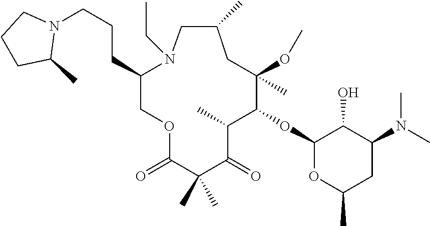 |
| 197 | 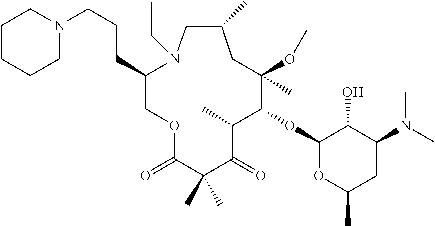 |
| 198 | 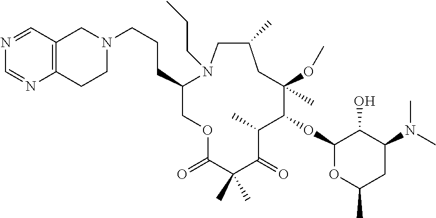 |
| 199 | 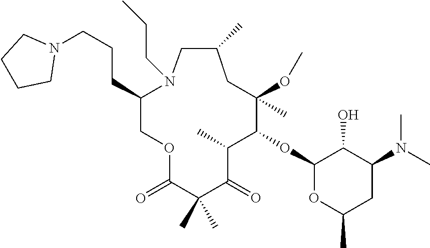 |
| 200 | 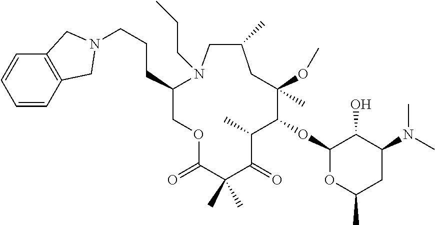 |
| 201 | 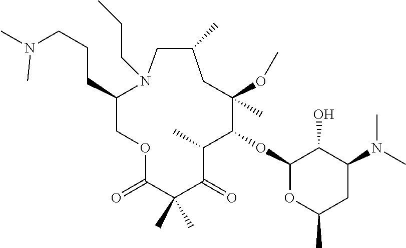 |
| 202 | 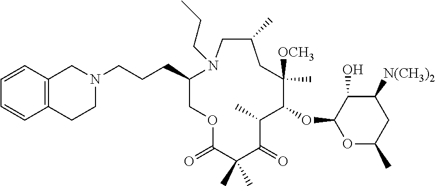 |
| 203 | 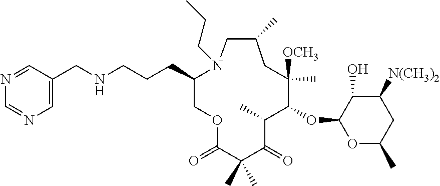 |
| 204 | 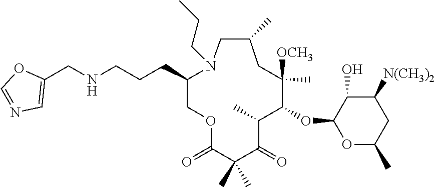 |
| 205 | 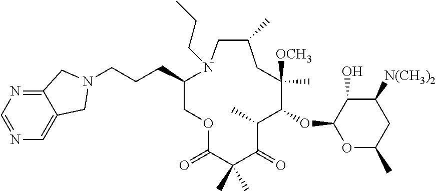 |
| 206 | 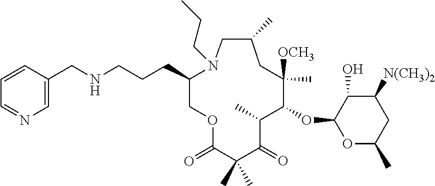 |
| 207 | 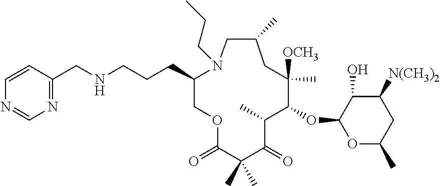 |
| 208 | 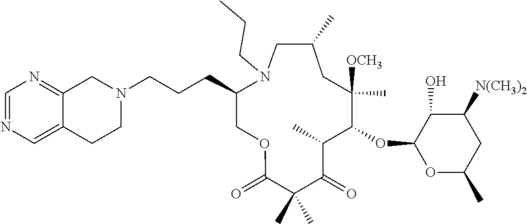 |
| 209 | 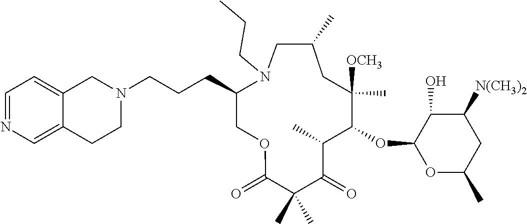 |
| 210 | 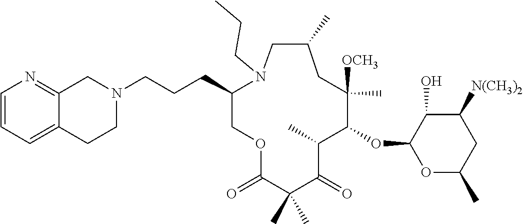 |
| 211 | 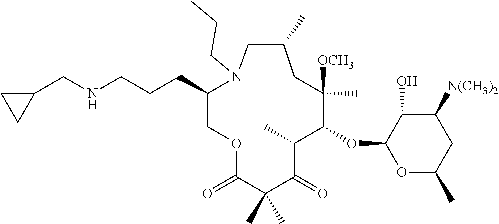 |
| 212 | 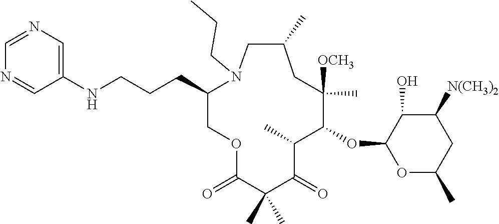 |
| 213 | 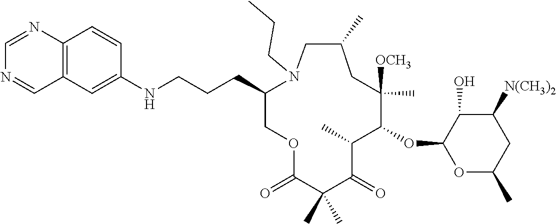 |
| 214 | 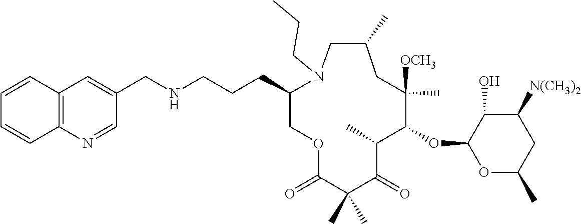 |
| 215 | 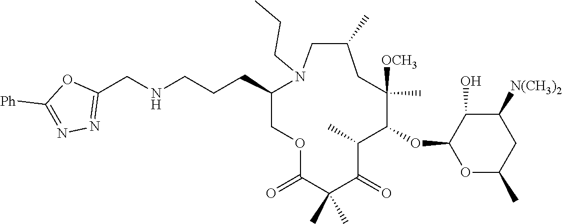 |
| 216 | 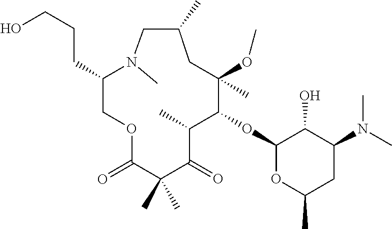 |
| 217 | 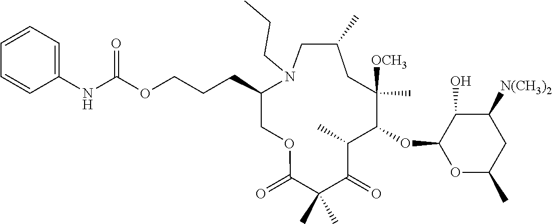 |
| 218 | 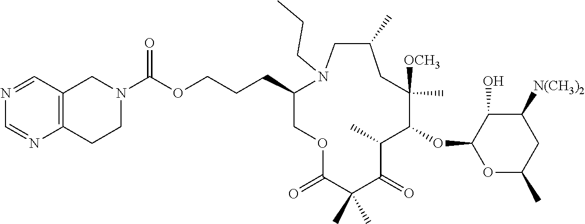 |
| 219 | 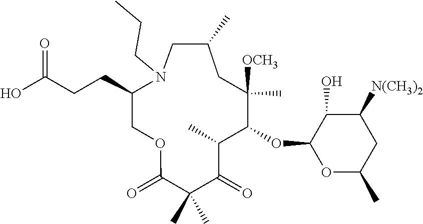 |
| 220 | 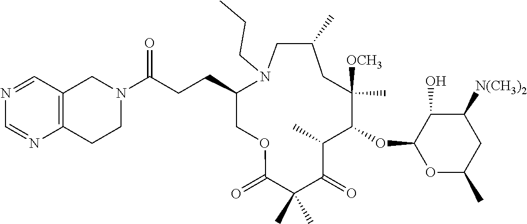 |
| 221 | 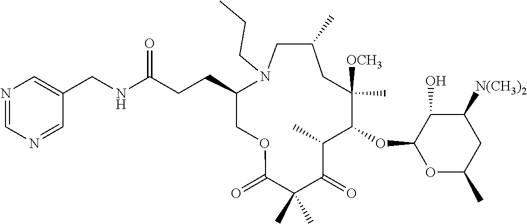 |
| 222 | 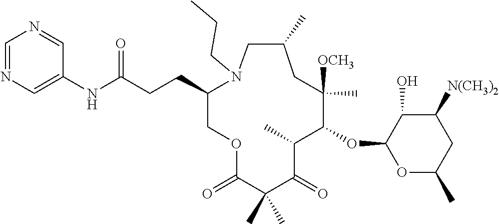 |
| 223 | 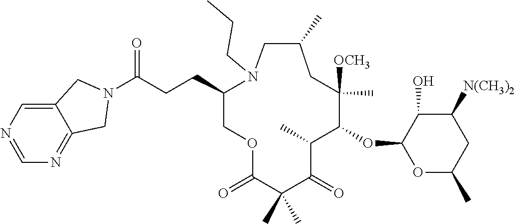 |
| 224 | 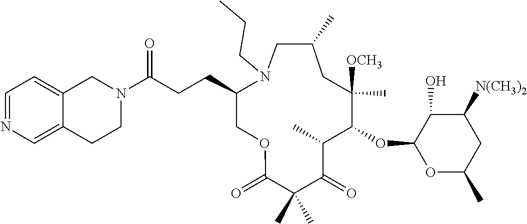 |
| 225 | 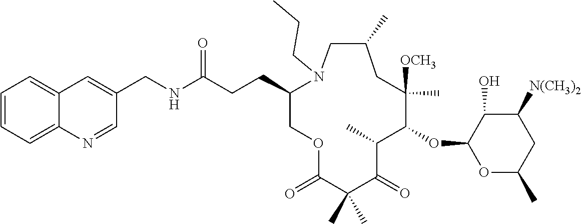 |
| 226 | 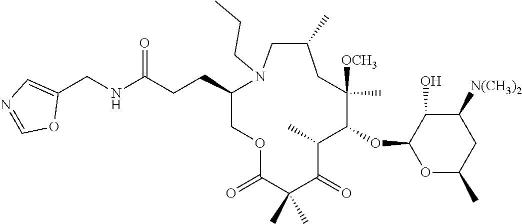 |
| 227 | 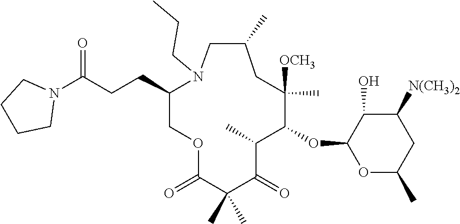 |
| 228 | 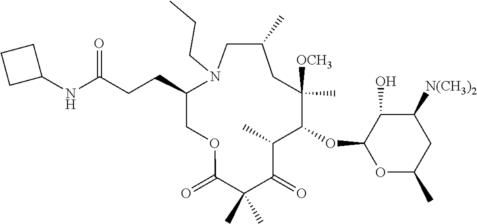 |
| 229 | 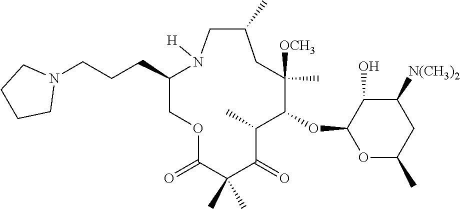 |
| 230 | 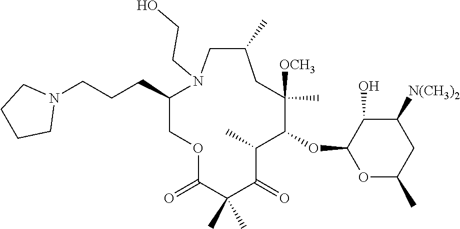 |
| 231 | 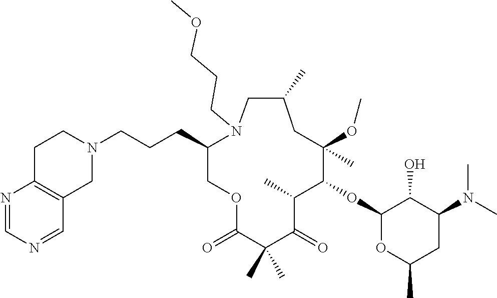 |
| 232 | 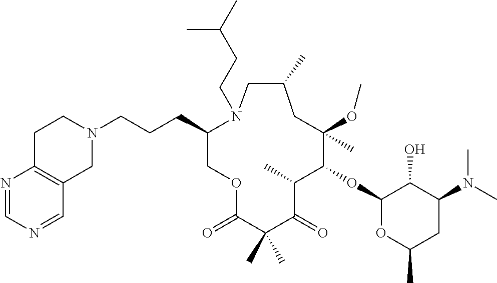 |
| 233 | 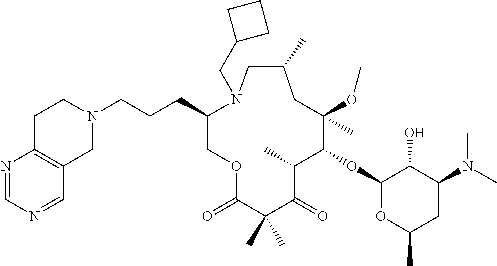 |
| 234 | 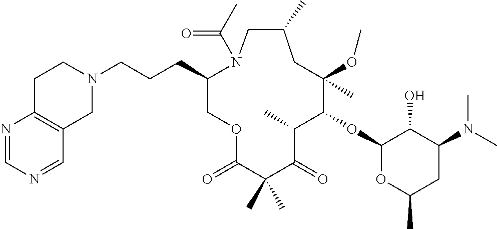 |
| 235 | 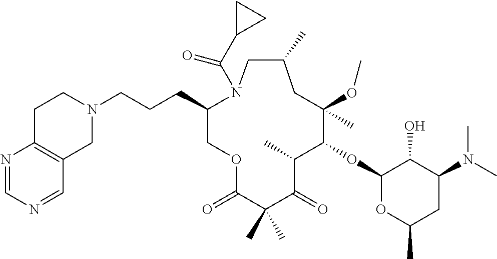 |
| 236 | 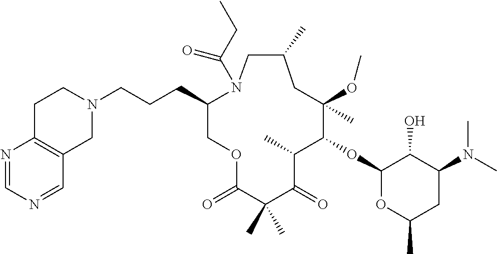 |
| 237 | 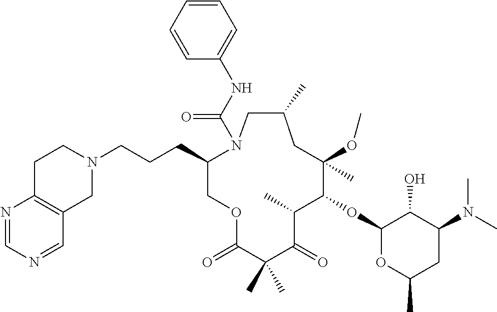 |
| 238 | 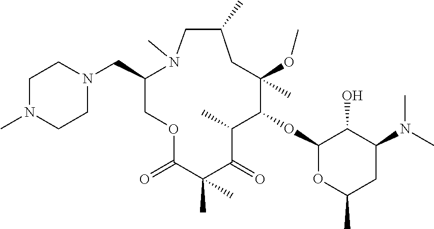 |
| 239 | 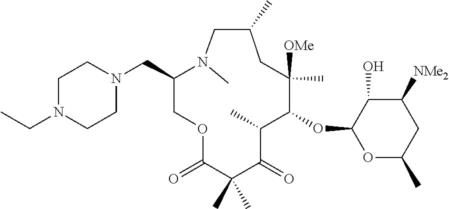 |
| 240 | 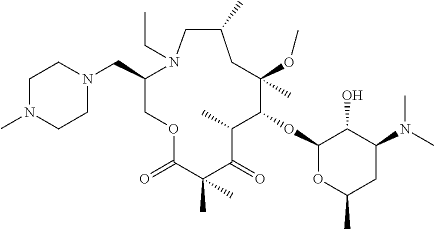 |
| 241 | 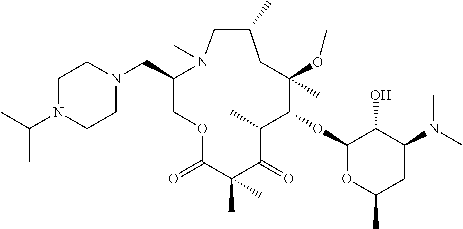 |
| 242 | 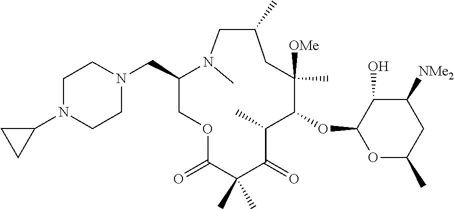 |
| 243 | 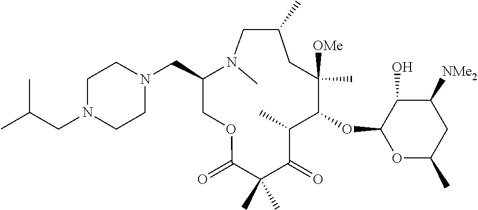 |
| 244 | 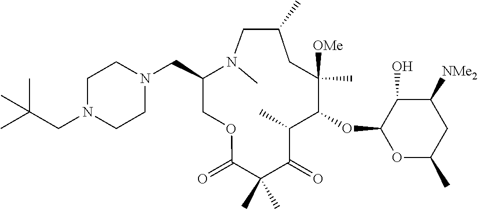 |
| 245 | 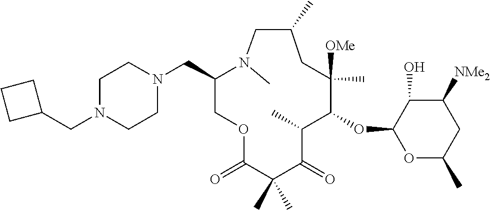 |
| 246 | 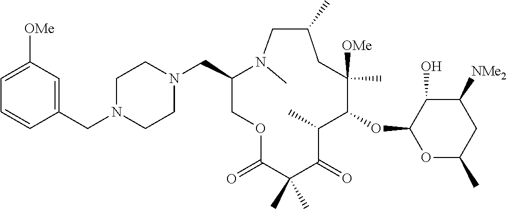 |
| 247 | 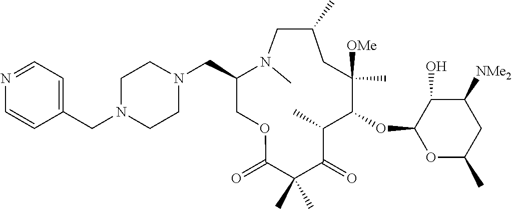 |
| 248 | 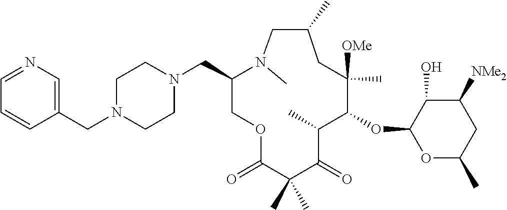 |
| 249 | 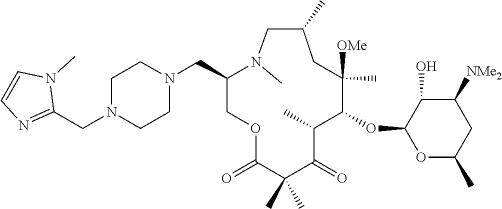 |
| 250 | 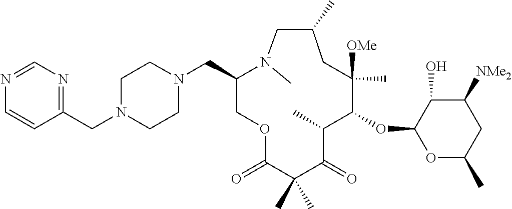 |
| 251 | 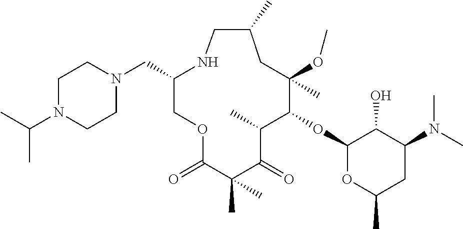 |
| 252 | 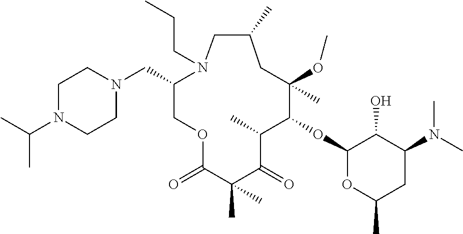 |
| 253 | 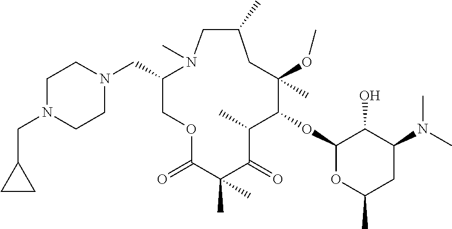 |
| 254 | 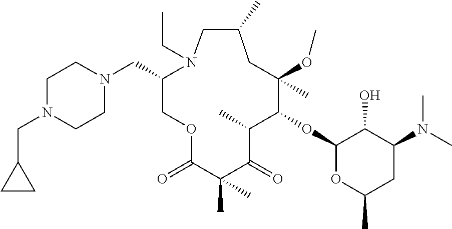 |
| 255 | 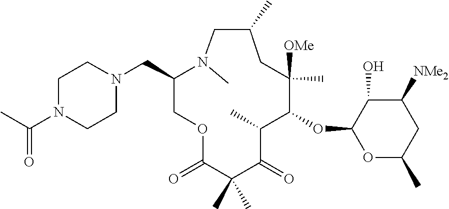 |
| 256 | 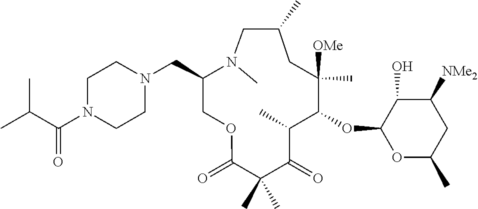 |
| 258 | 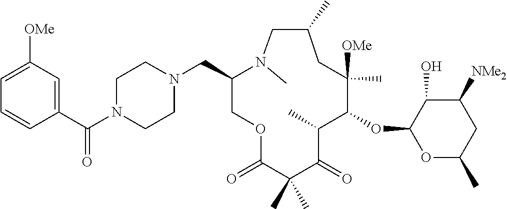 |
| 258 | 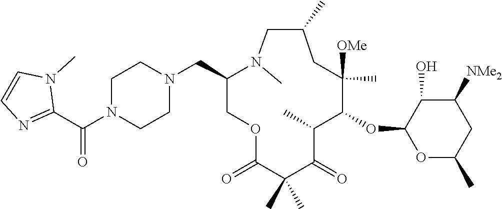 |
| 259 | 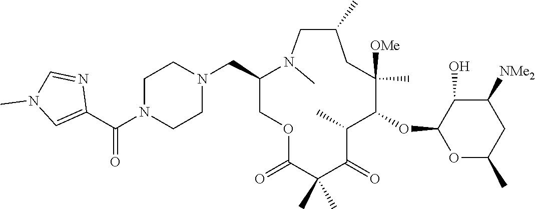 |
| 260 | 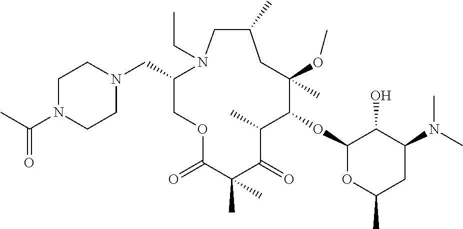 |
| 261 | 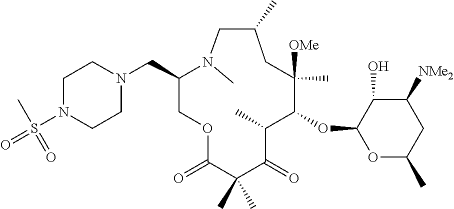 |
| 262 | 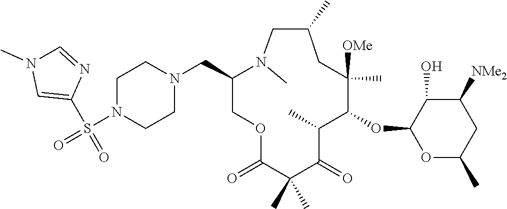 |
| 263 | 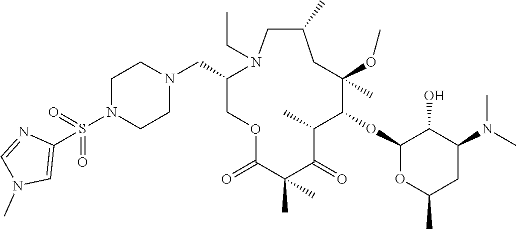 |
| 264 | 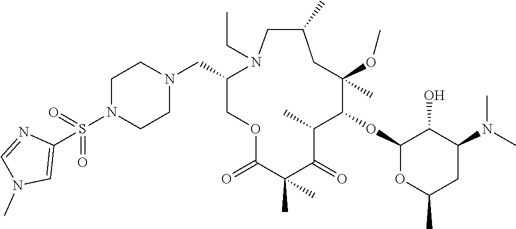 |
| 265 | 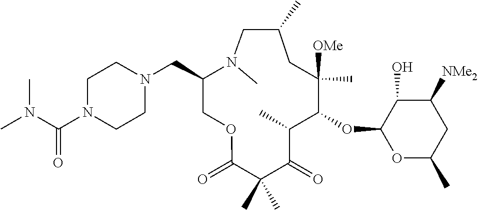 |
| 266 | 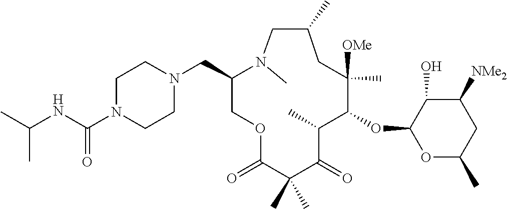 |
| 267 | 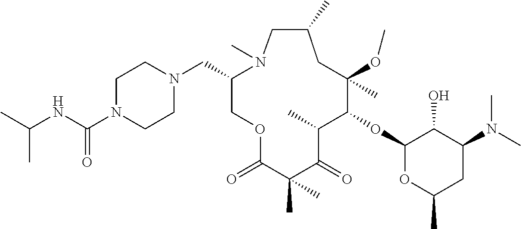 |
| 268 | 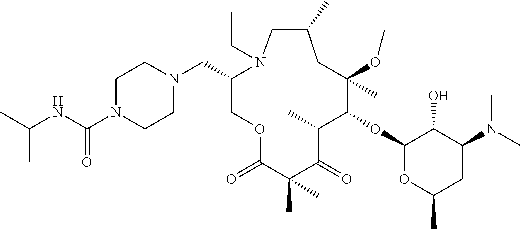 |
| 269 | 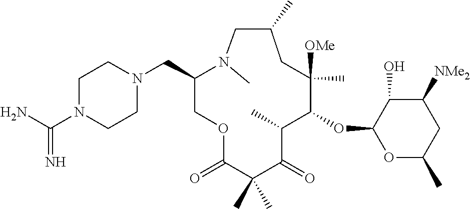 |
| 270 | 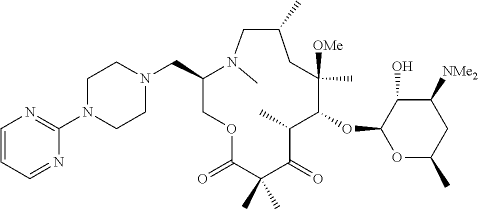 |
| 271 | 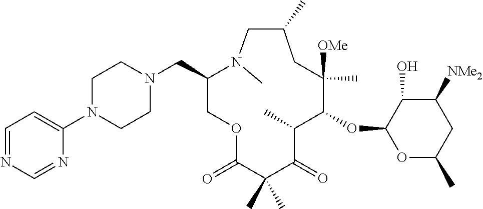 |
| 272 | 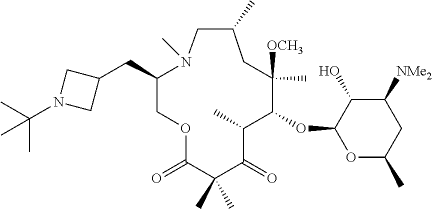 |
| 273 | 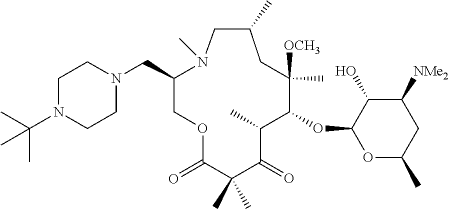 |
| 274 | 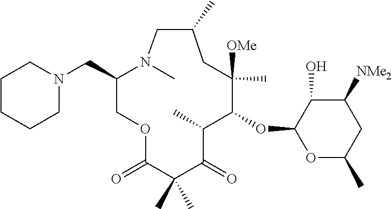 |
| 275 | 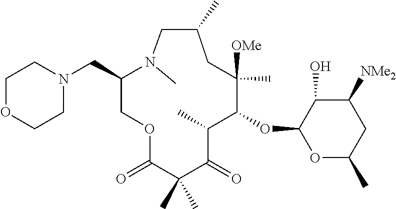 |
| 276 | 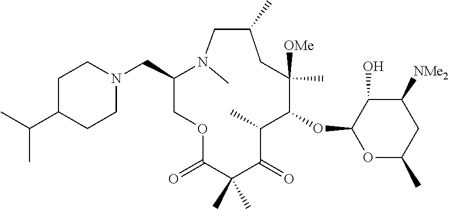 |
| 277 | 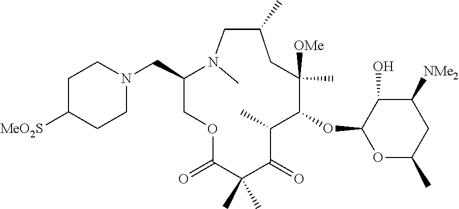 |
| 278 | 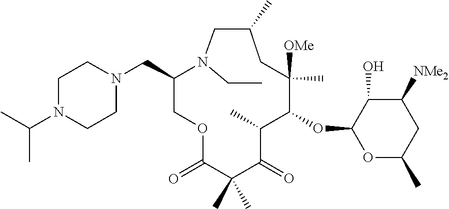 |
| 279 | 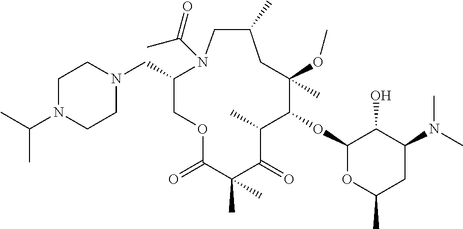 |
| 280 | 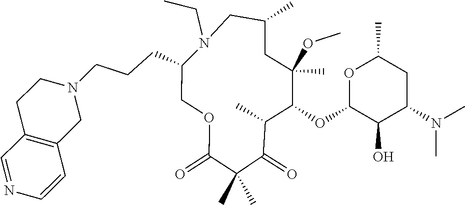 |
| 281 | 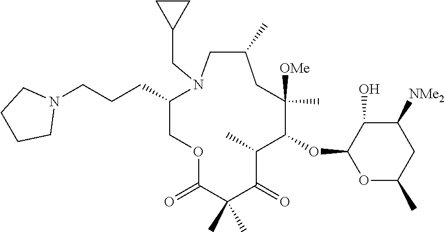 |
| 282 | 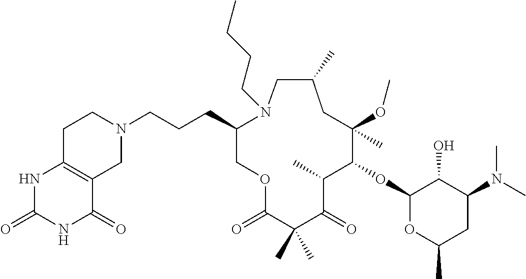 |

In another aspect, what is provided is a compound as depicted in Table B.

TABLE B
Compounds

| Cmpd # | Structure |
|---|---|
| 7 | 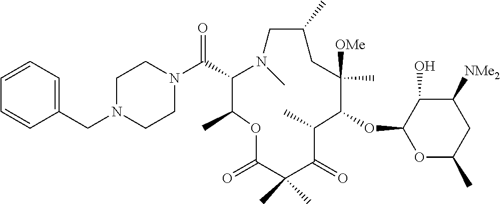 |
| 8 | 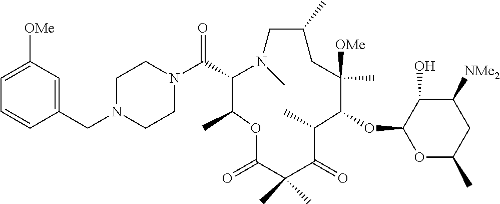 |
| 9 | 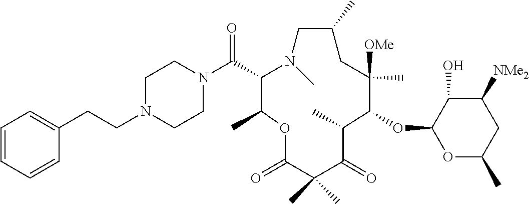 |
| 10 | 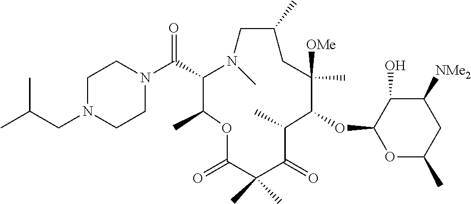 |
| 11 | 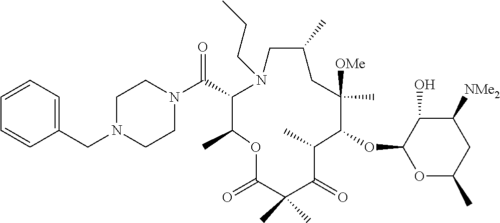 |
| 12 | 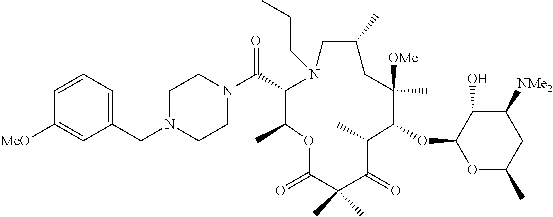 |
| 13 | 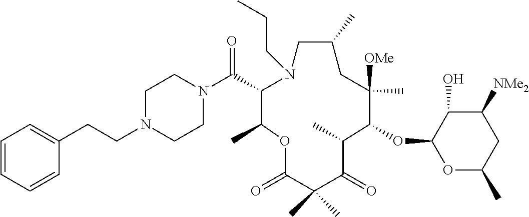 |
| 14 | 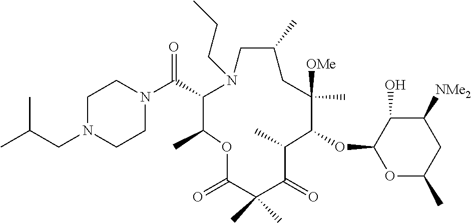 |
| 15 | 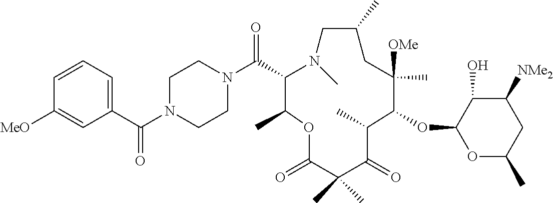 |
| 16 | 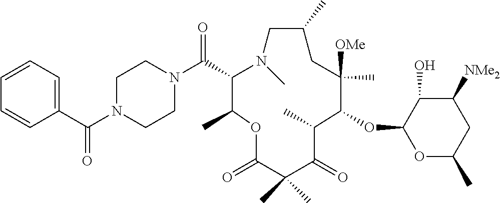 |
| 17 | 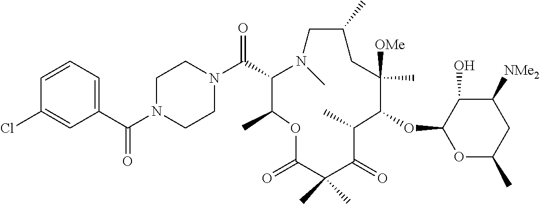 |
| 18 | 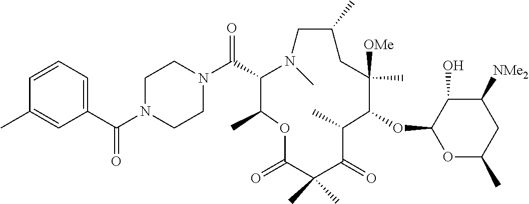 |
| 19 | 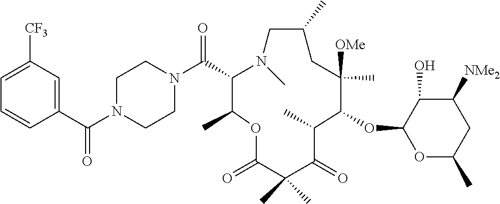 |
| 20 | 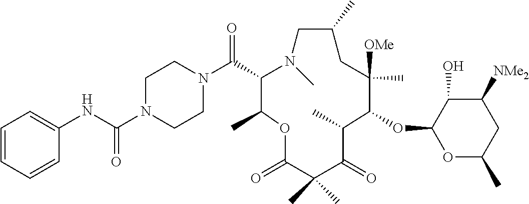 |
| 21 | 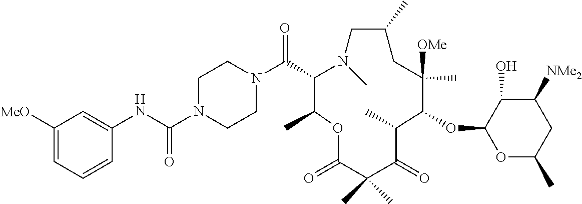 |
| 22 | 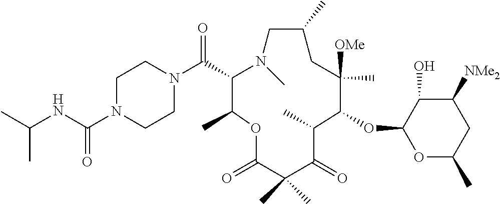 |
| 23 | 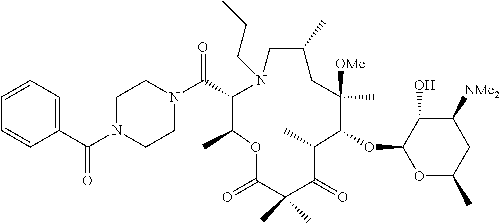 |
| 24 | 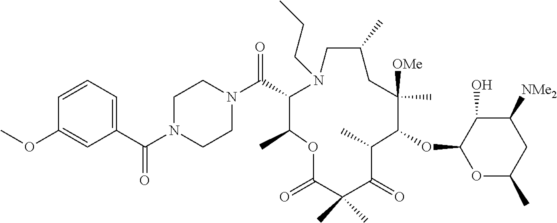 |
| 25 | 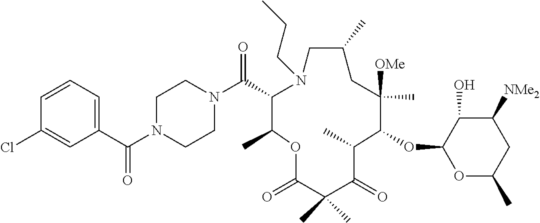 |
| 26 | 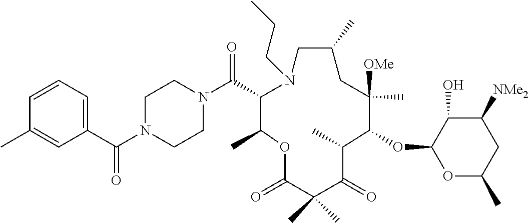 |
| 27 | 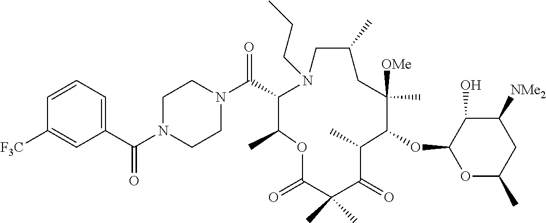 |
| 28 | 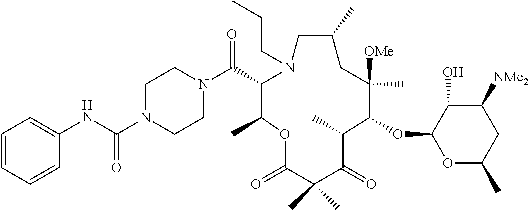 |
| 29 | 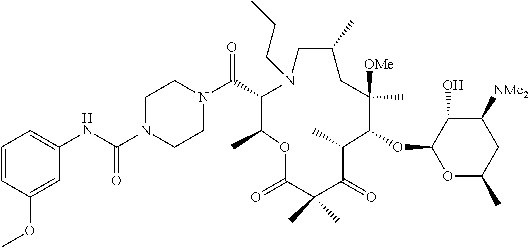 |
| 30 | 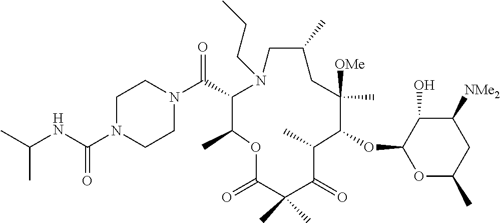 |
| 31 | 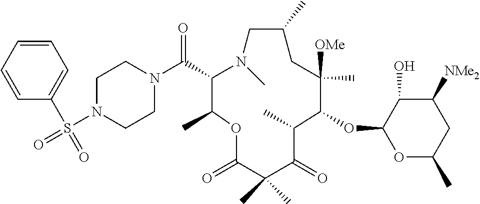 |
| 32 | 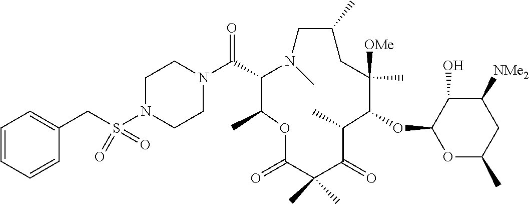 |
| 33 | 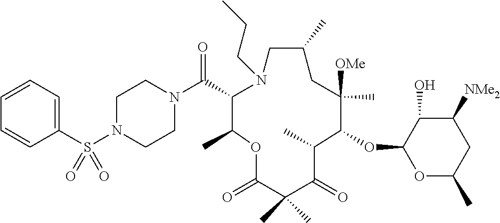 |
| 34 | 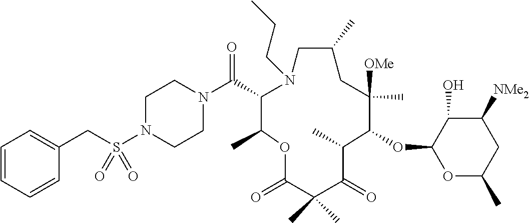 |
| 36 | 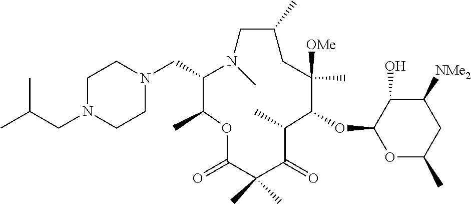 |
| 37 | 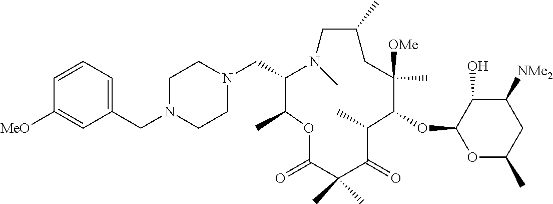 |
| 38 | 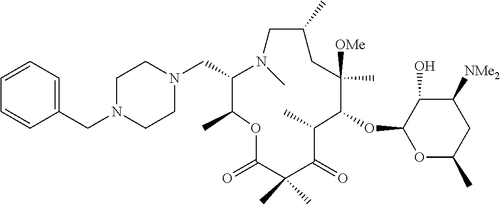 |
| 39 | 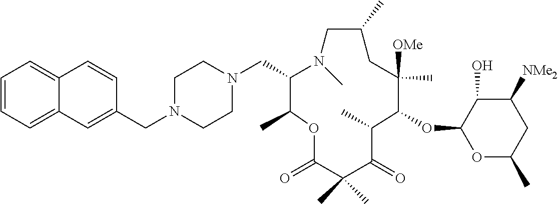 |
| 40 | 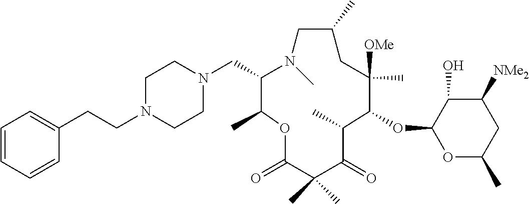 |
| 41 | 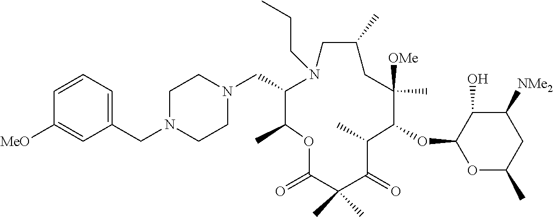 |
| 42 | 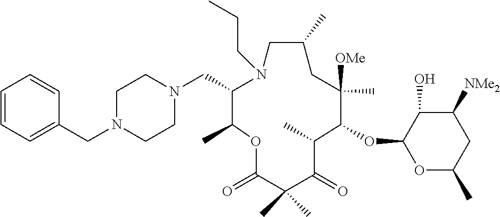 |
| 43 | 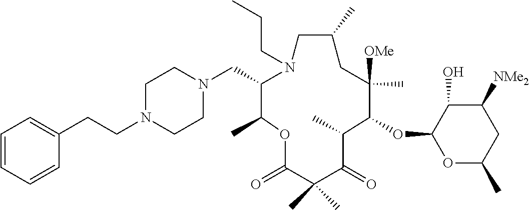 |
| 44 | 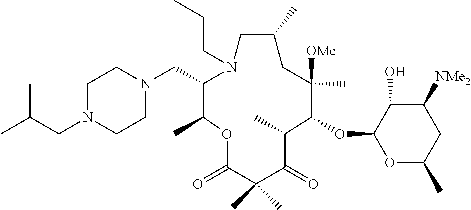 |
| 49 | 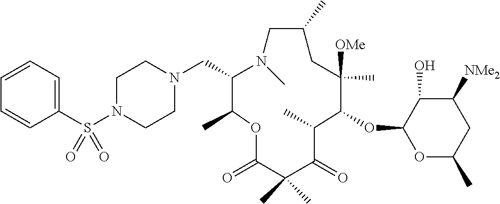 |
| 50 | 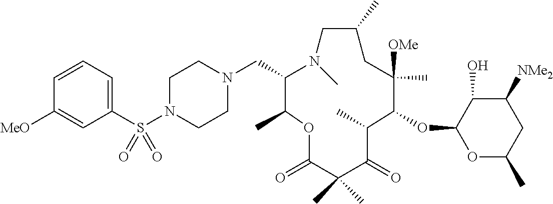 |
| 51 | 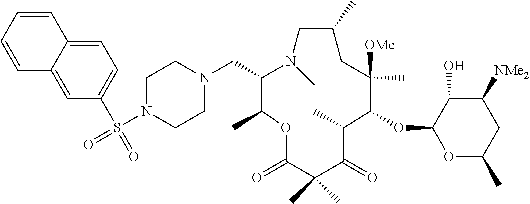 |
| 52 | 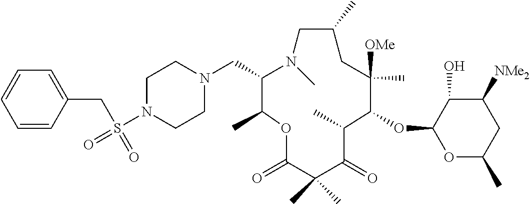 |
| 53 | 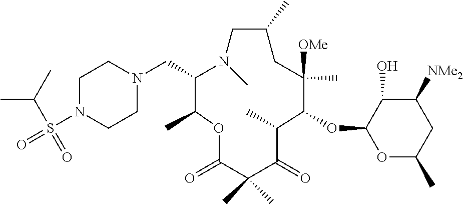 |
| 54 | 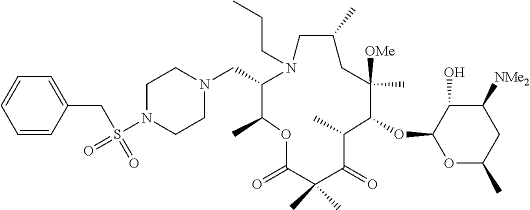 |
| 61 | 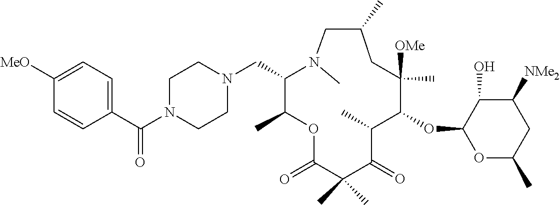 |
| 62 | 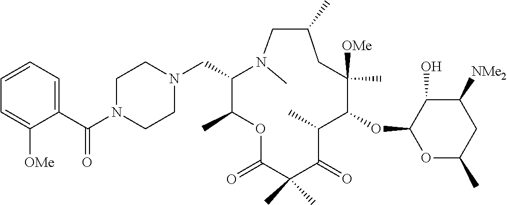 |
| 63 | 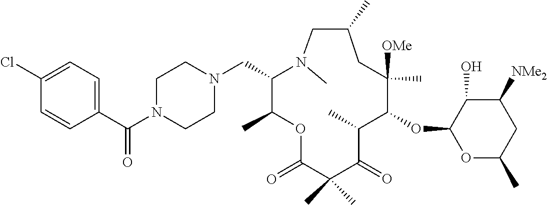 |
| 64 | 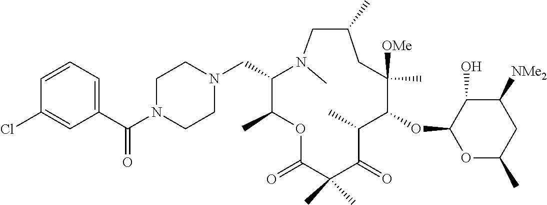 |
| 65 | 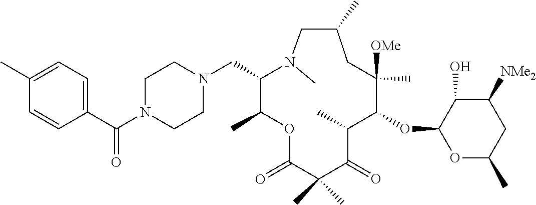 |
| 66 | 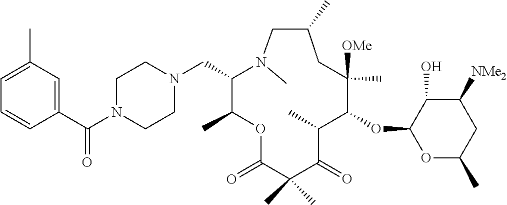 |
| 67 | 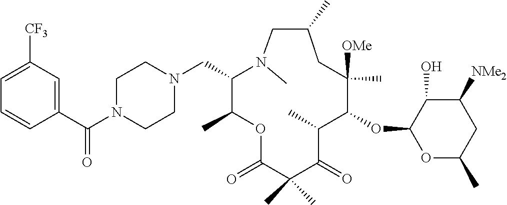 |
| 68 | 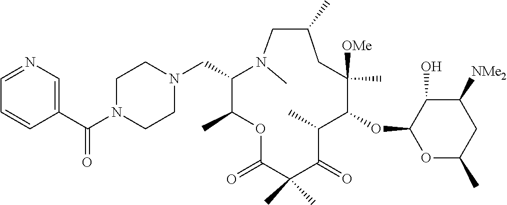 |
| 69 | 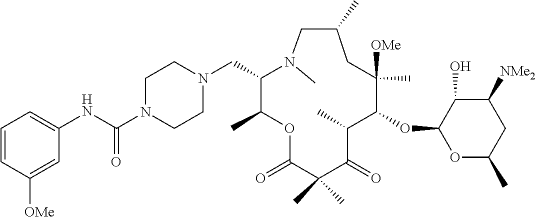 |
| 70 | 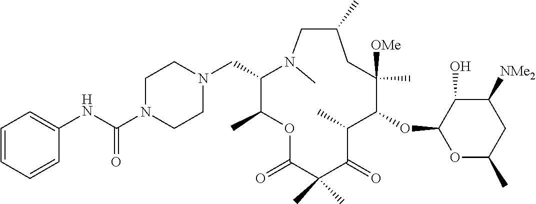 |
| 71 | 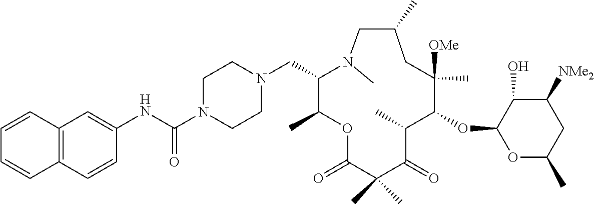 |
| 72 | 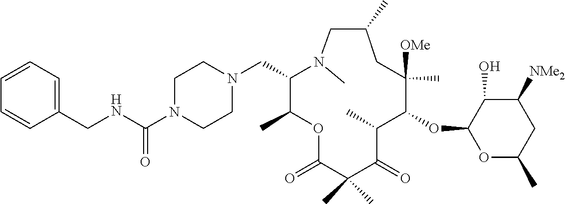 |
| 73 | 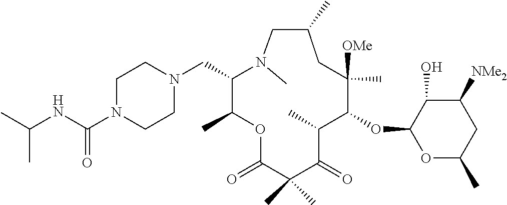 |
| 74 | 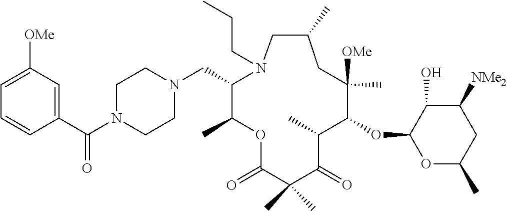 |
| 75 | 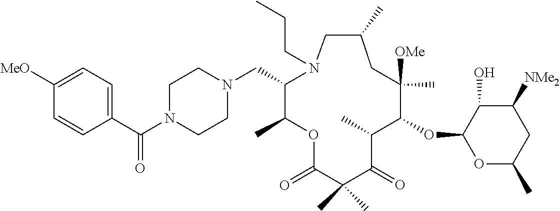 |
| 76 | 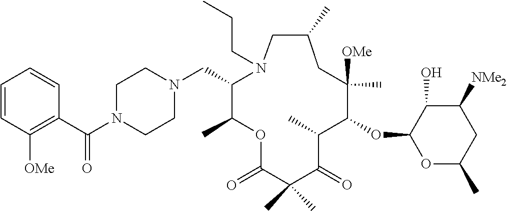 |
| 77 | 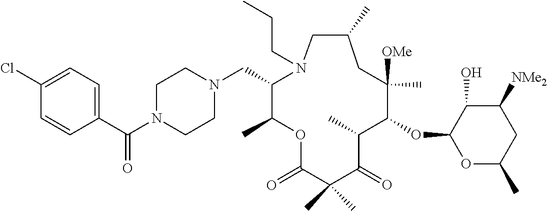 |
| 78 | 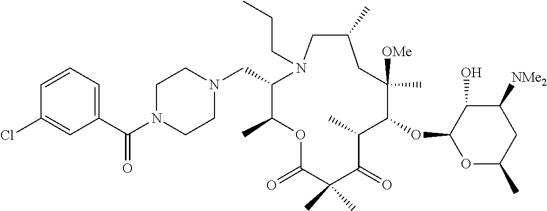 |
| 79 | 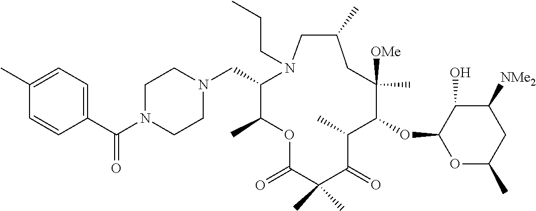 |
| 80 | 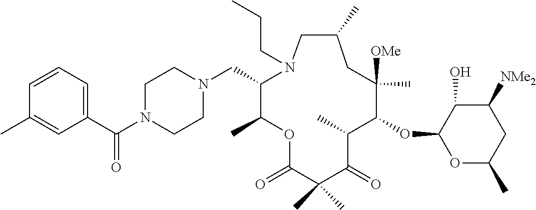 |
| 81 | 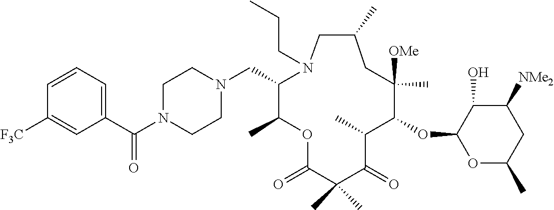 |
| 82 | 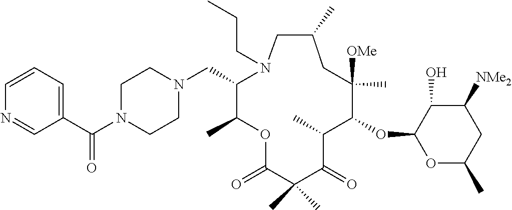 |
| 83 | 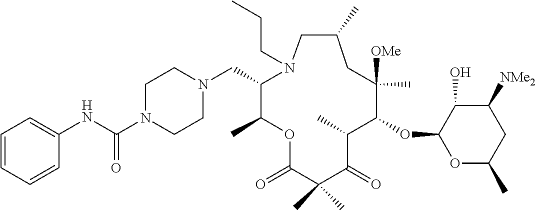 |
| 89 | 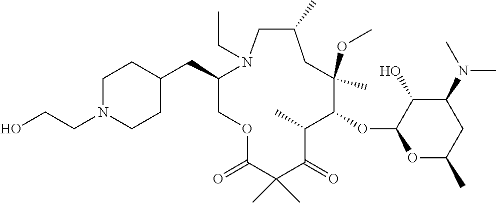 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@H]1CC1CCN(CCO)CC1] |
| 91 | 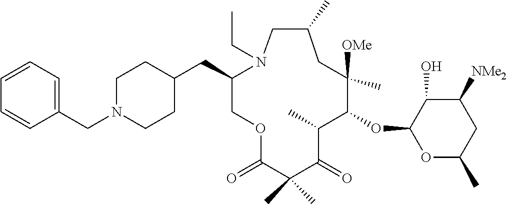 |
| 92 | 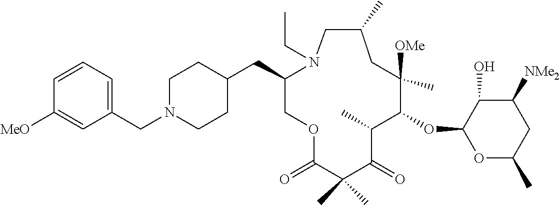 |
| 93 | 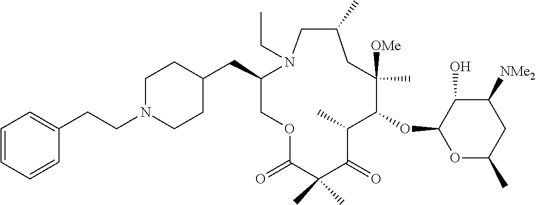 |
| 94 | 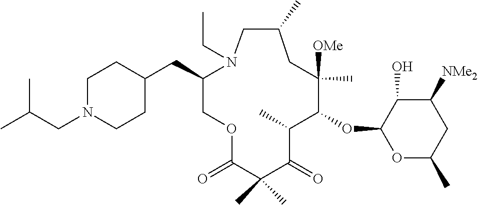 |
| 95 | 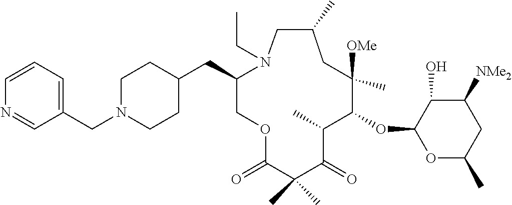 |
| 96 | 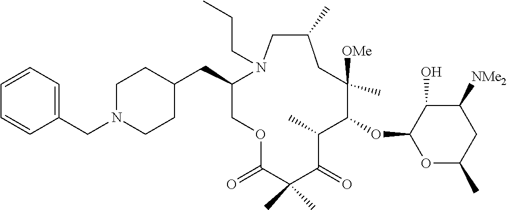 |
| 97 | 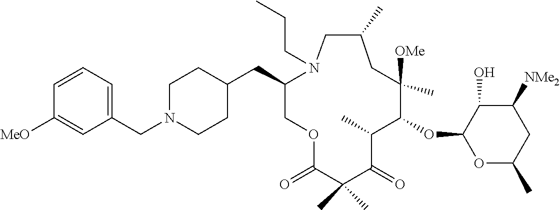 |
| 98 | 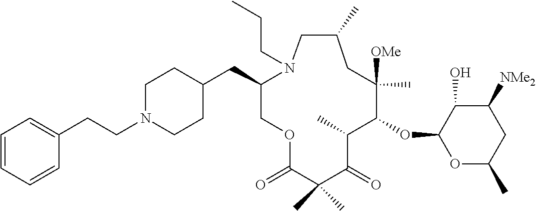 |
| 99 | 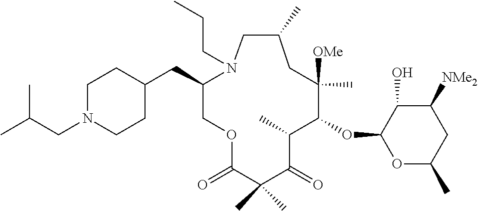 |
| 100 | 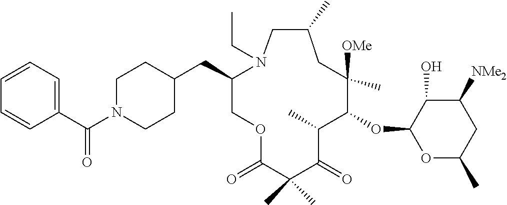 |
| 101 | 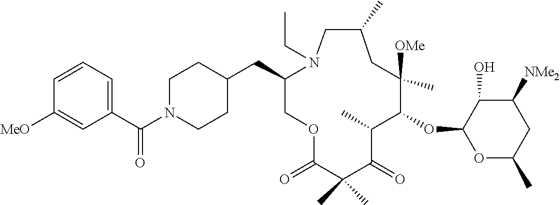 |
| 102 | 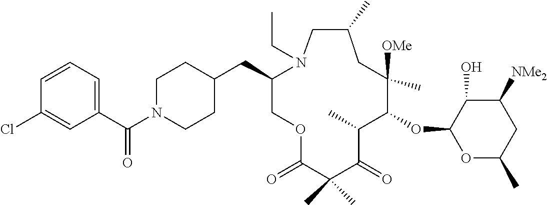 |
| 103 | 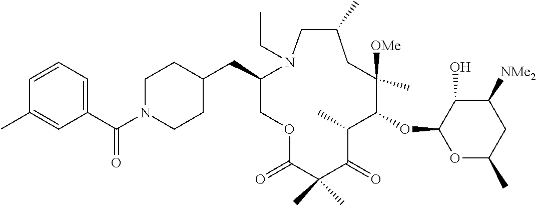 |
| 104 | 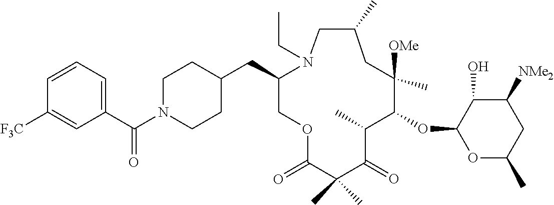 |
| 105 | 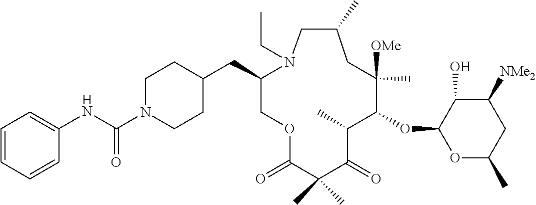 |
| 106 | 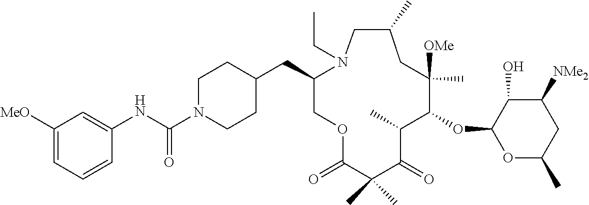 |
| 107 | 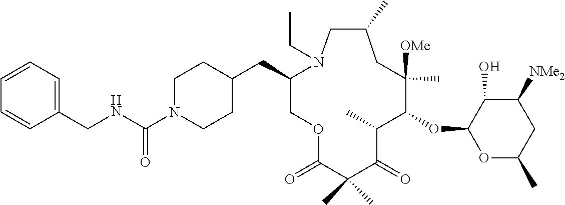 |
| 108 | 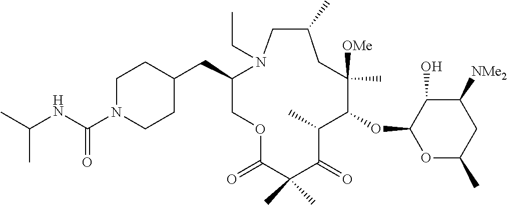 |
| 109 | 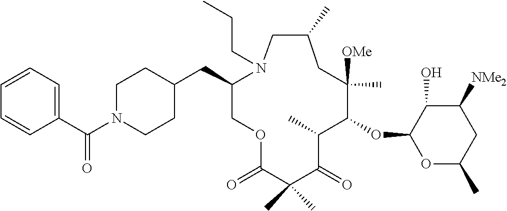 |
| 110 | 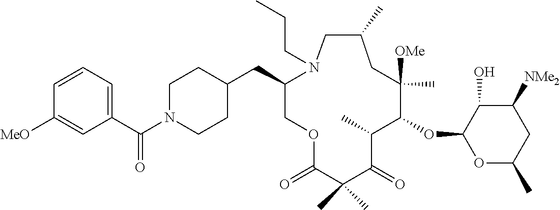 |
| 111 | 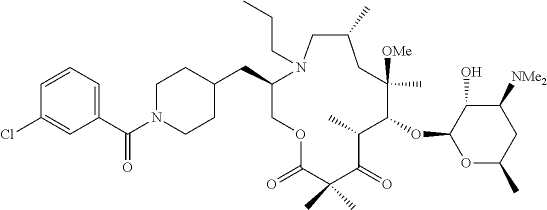 |
| 112 | 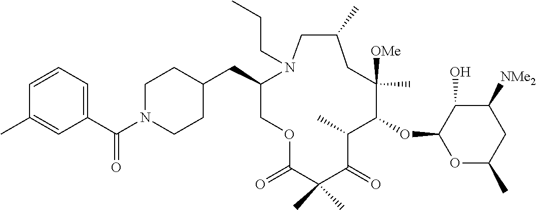 |
| 113 | 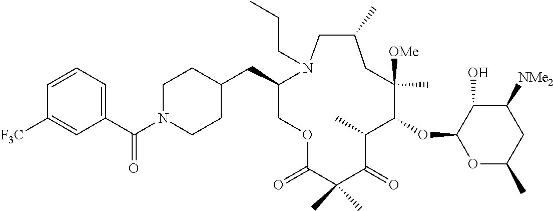 |
| 114 | 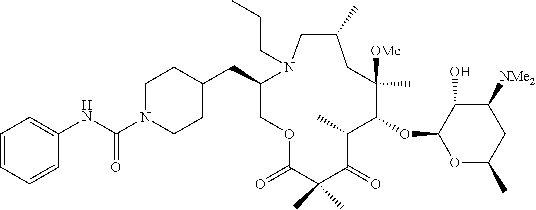 |
| 115 | 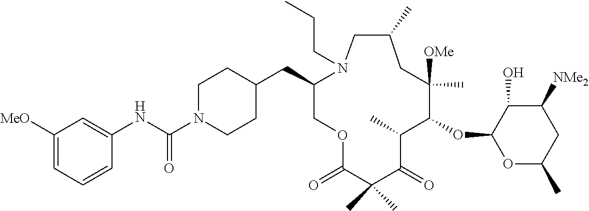 |
| 116 | 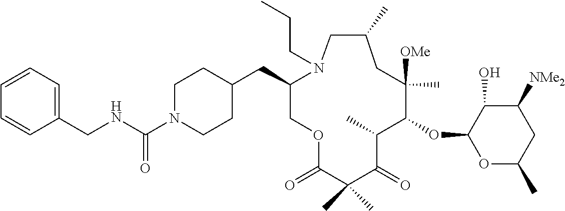 |
| 117 | 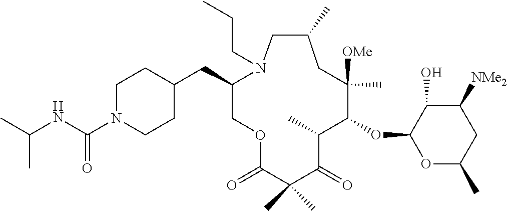 |
| 118 | 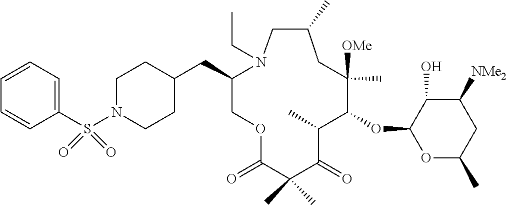 |
| 119 | 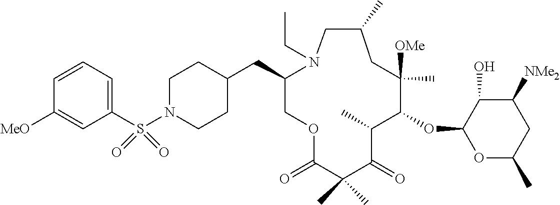 |
| 120 | 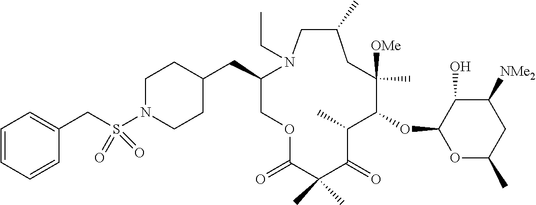 |
| 121 | 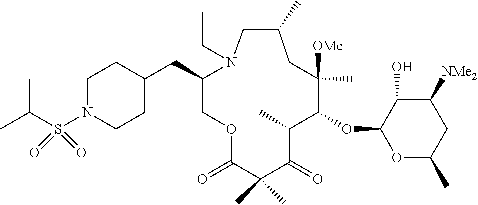 |
| 122 | 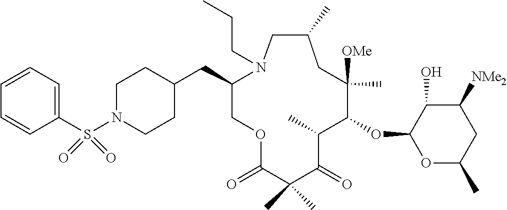 |
| 135 | 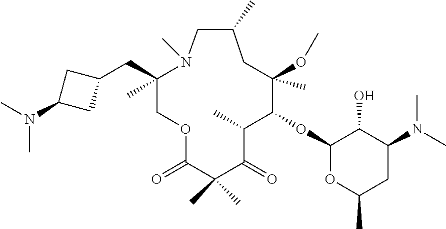 |
| 167 | 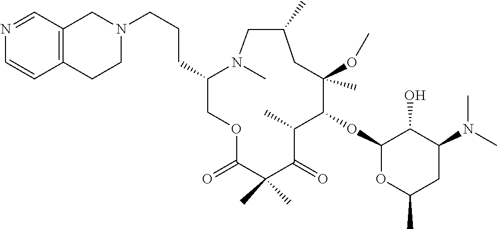 |
| 172 | 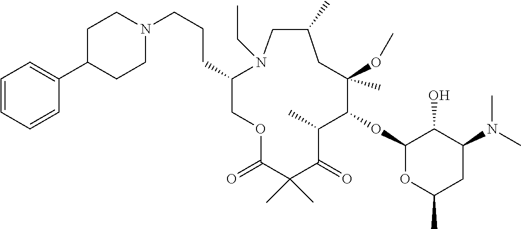 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCC(c2ccccc2)CC1] |
| 173 | 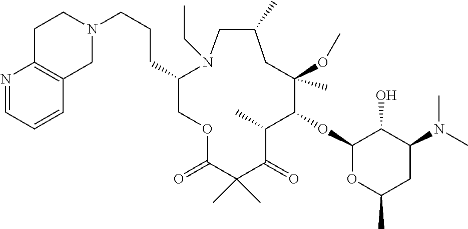 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCc2ncccc2C1] |
| 174 | 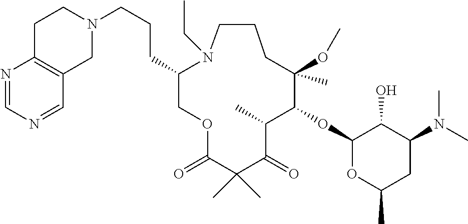 [SMILES: CCN1CCC[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCc2ncncc2C1] |
| 175 | 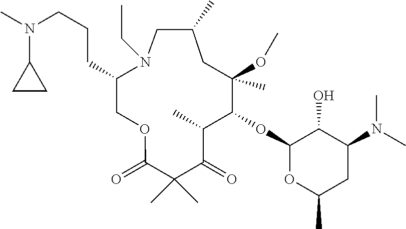 |
| 176 | 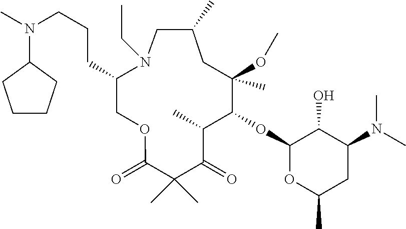 |
| 179 | 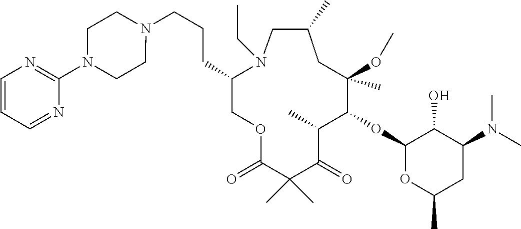 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCN(c2ncccn2)CC1] |
| 180 | 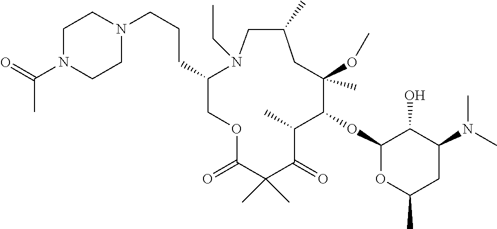 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCN(C(C)=O)CC1] |
| 184 | 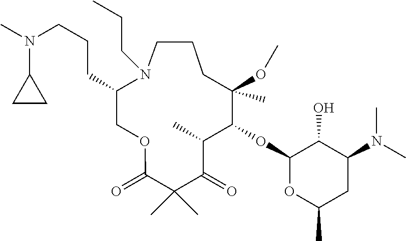 |
| 185 | 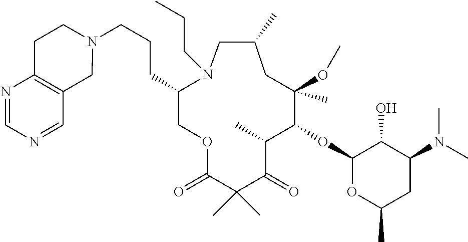 [SMILES: CCCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCc2ncncc2C1] |
| 186 | 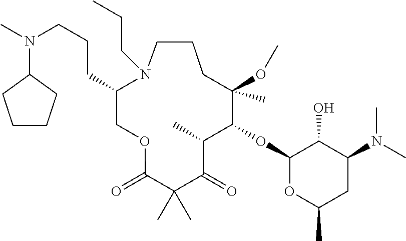 |
| 192 | 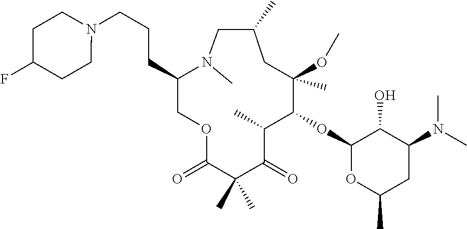 |
| 194 | 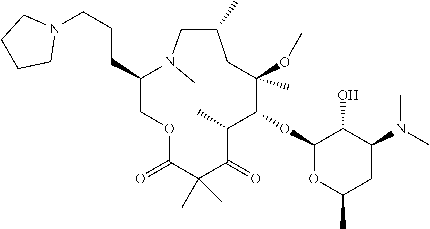 [SMILES: CO[C@]1(C)C[C@@H](C)CN(C)[C@H](CCCN2CCCC2)COC(=O)C(C)(C)C(=O)[C@H](C)[C@H]1O[C@@H]1O[C@H](C)C[C@H](N(C)C)[C@H]1O] |
| 202 | 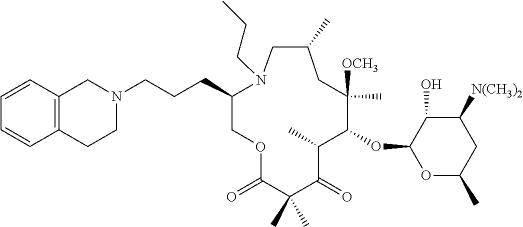 |
| 203 | 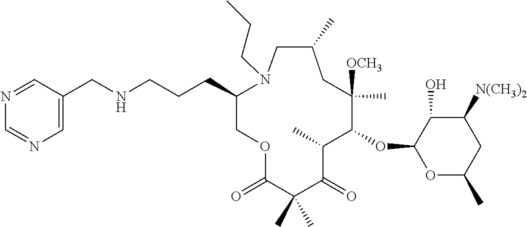 |
| 204 | 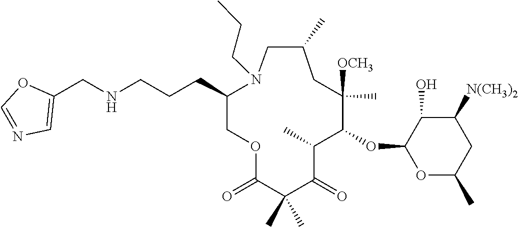 |
| 205 | 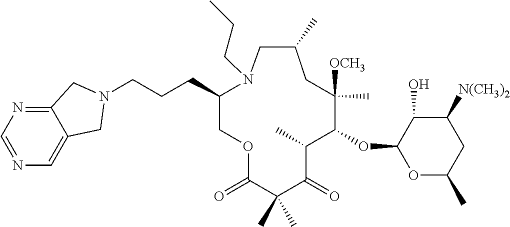 |
| 206 | 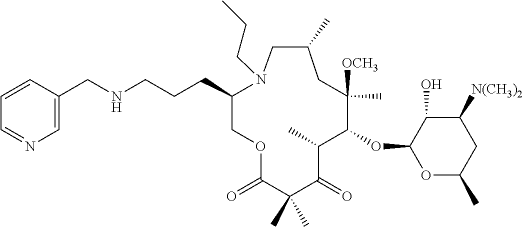 |
| 207 | 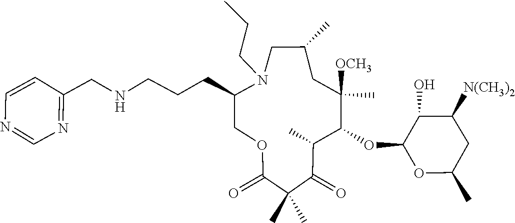 |
| 208 | 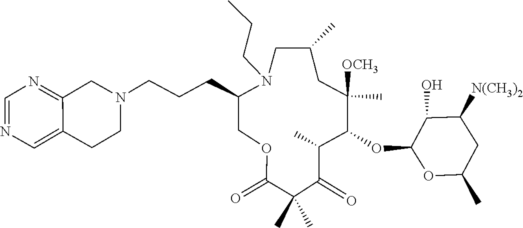 |
| 209 | 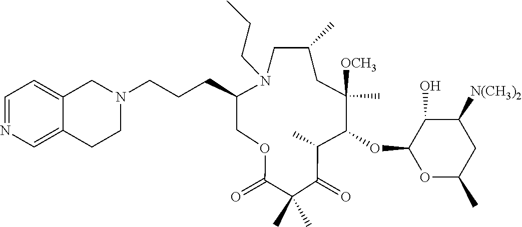 |
| 210 | 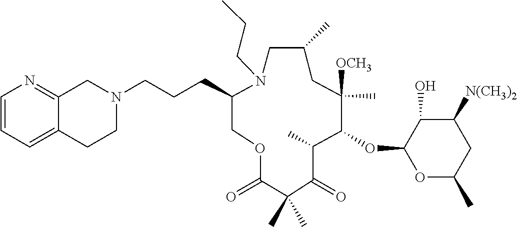 |
| 211 | 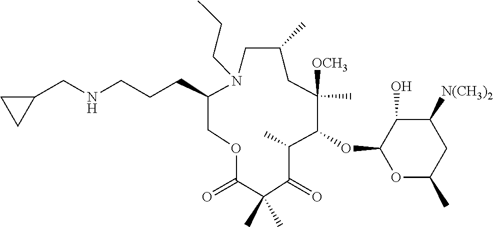 |
| 212 | 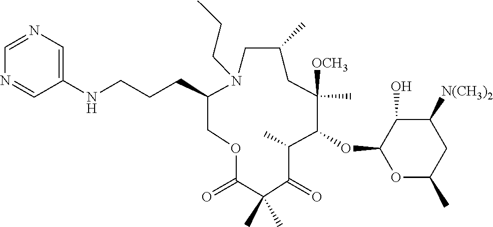 |
| 213 | 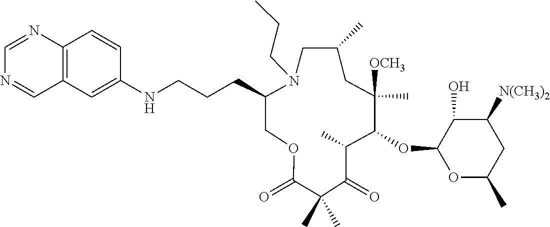 |
| 214 | 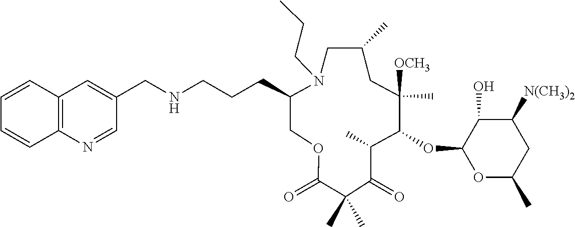 |
| 215 | 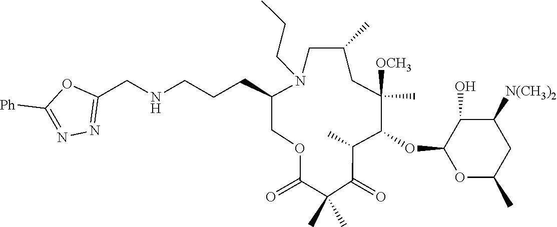 |
| 217 | 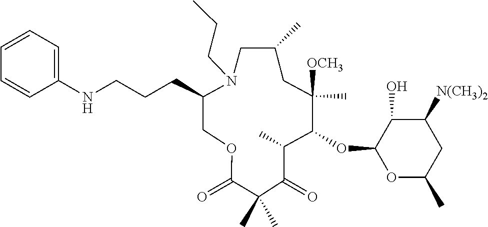 |
| 218 | 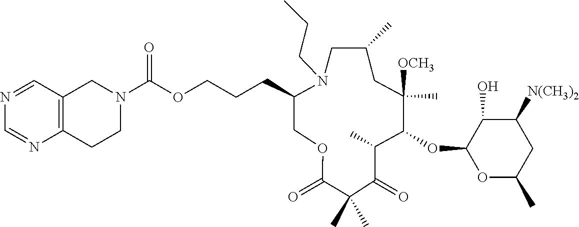 |
| 219 | 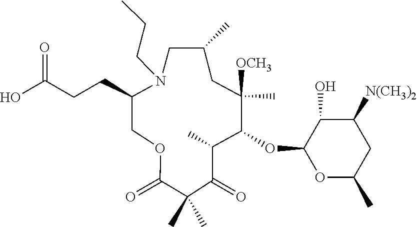 |
| 220 | 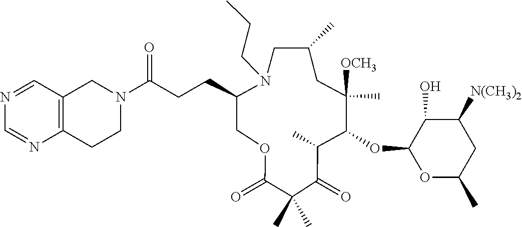 |
| 221 | 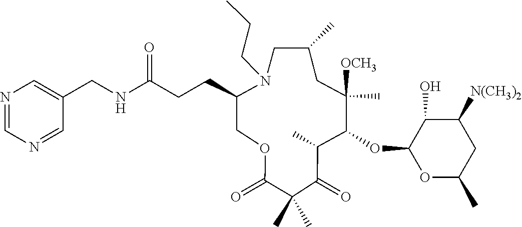 |
| 222 | 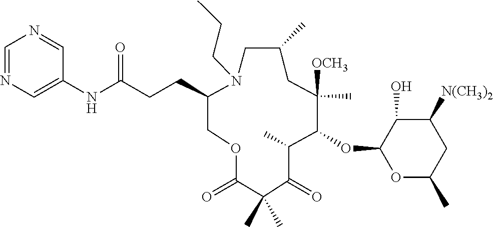 |
| 223 | 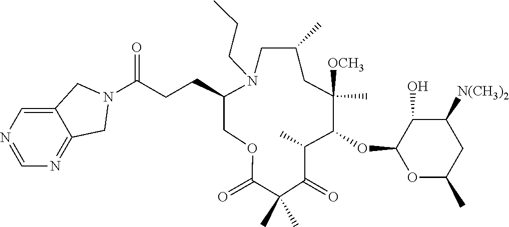 |
| 224 | 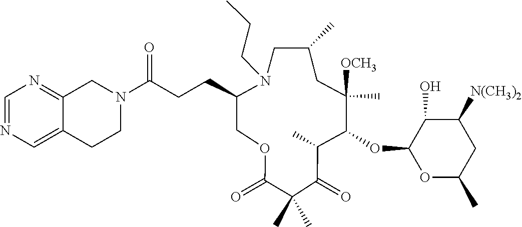 |
| 225 | 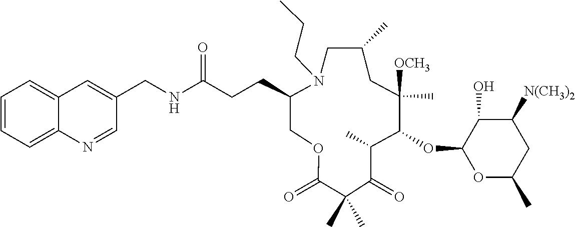 |
| 226 | 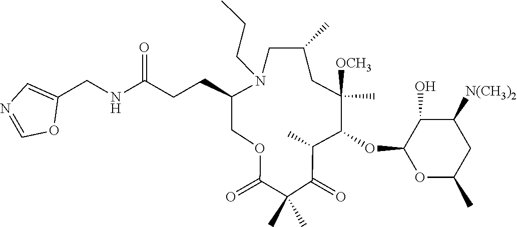 |
| 227 | 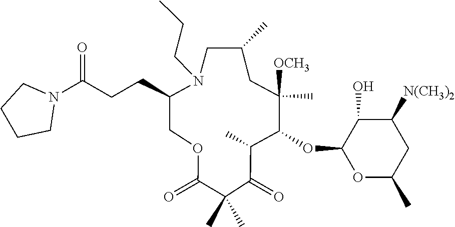 |
| 228 | 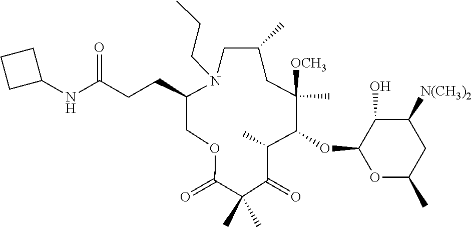 |
| 229 | 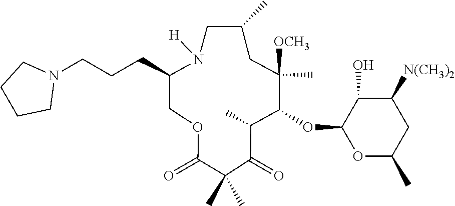 |
| 230 | 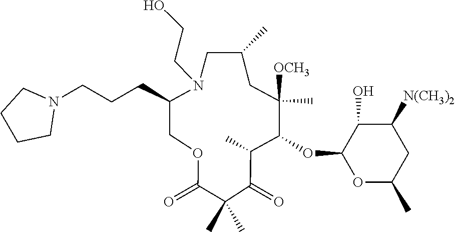 |
| 231 | 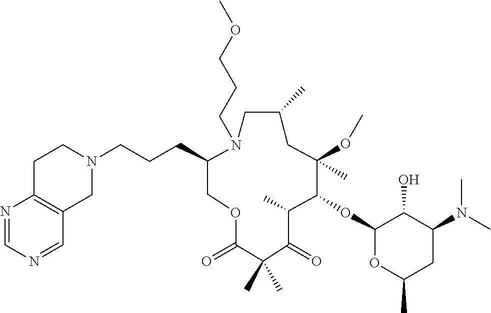 |
| 232 | 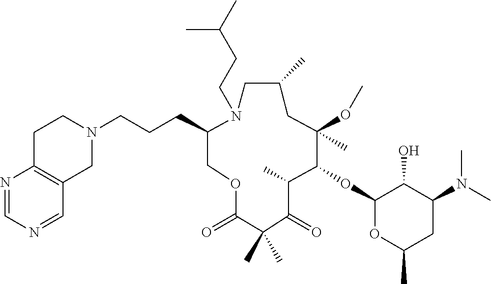 |
| 233 | 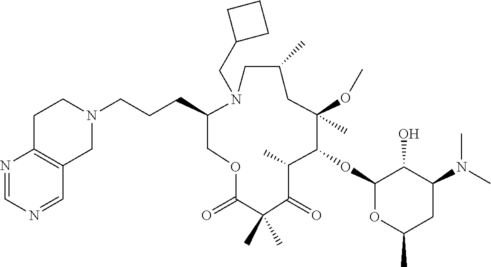 |
| 234 | 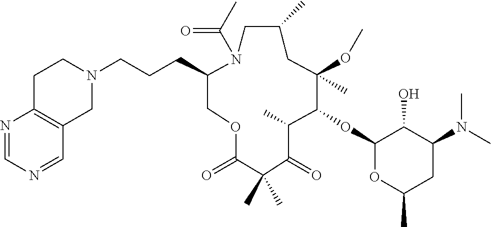 |
| 235 | 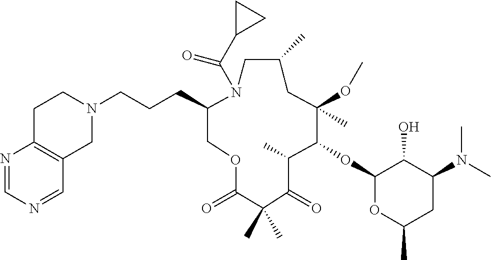 |
| 236 | 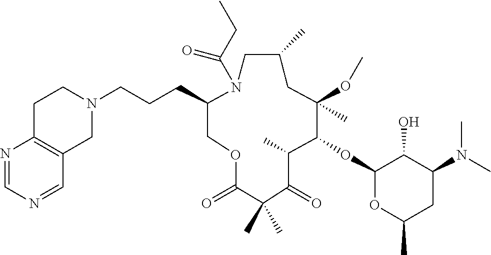 |
| 237 | 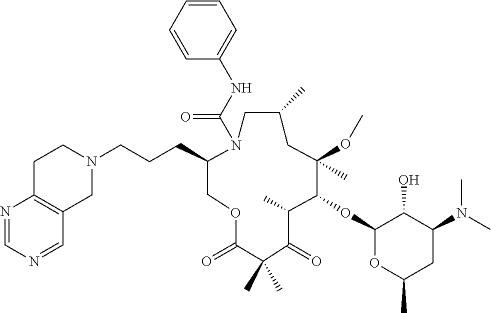 |
| 238 | 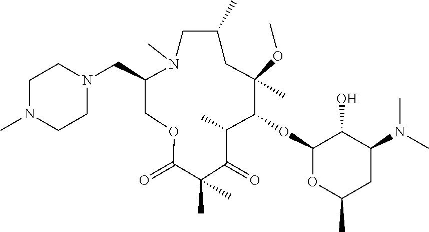 |
| 239 | 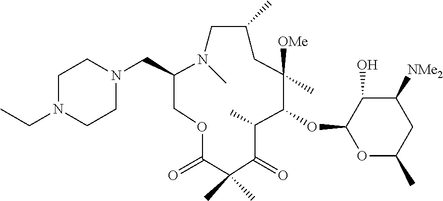 |
| 240 | 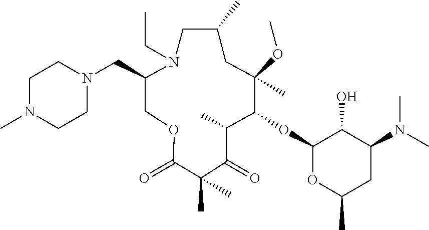 |
| 241 | 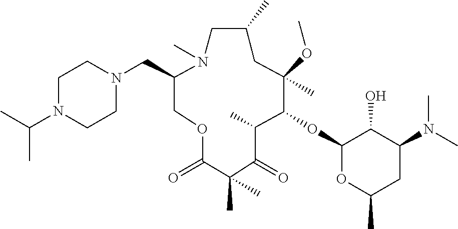 |
| 242 | 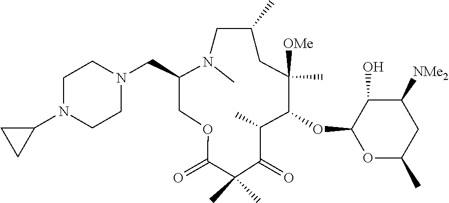 |
| 243 | 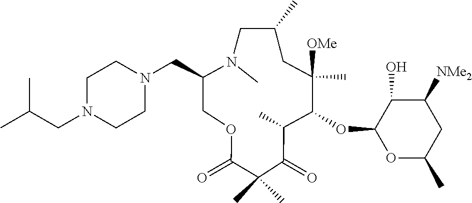 |
| 244 | 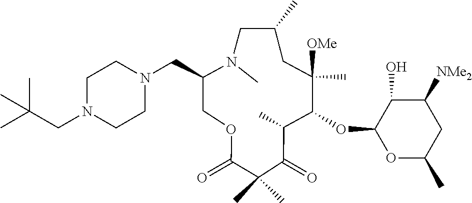 |
| 245 | 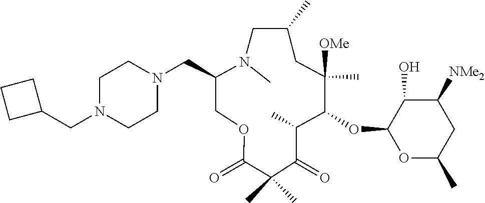 |
| 246 | 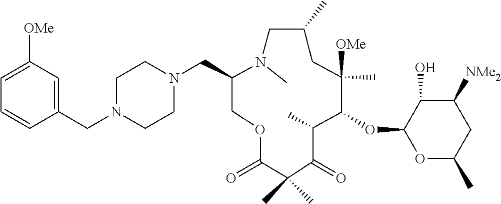 |
| 247 | 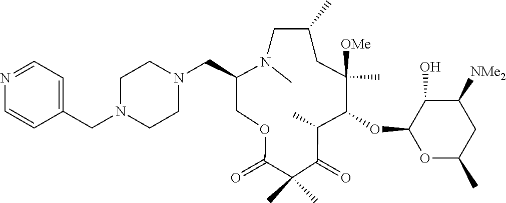 |
| 248 | 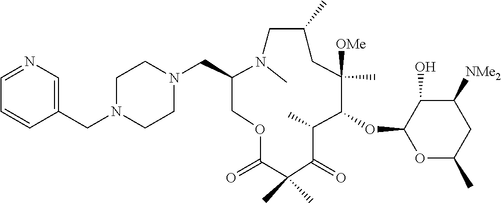 |
| 249 | 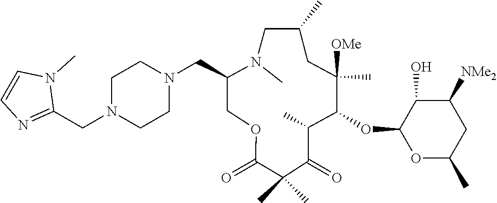 |
| 250 | 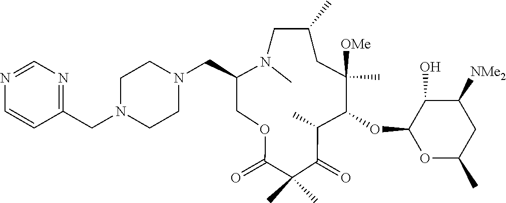 |
| 251 | 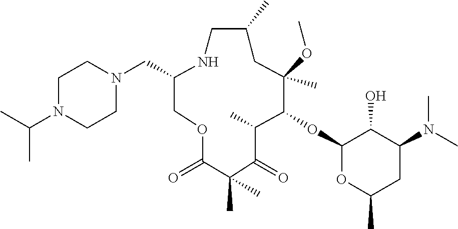 |
| 252 | 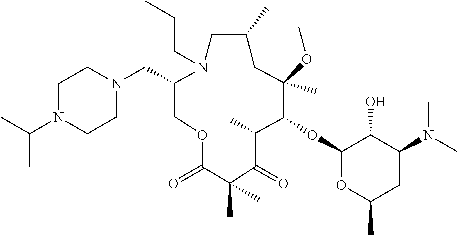 |
| 253 | 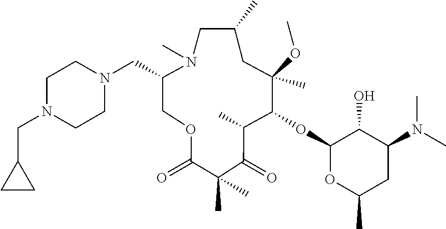 |
| 254 | 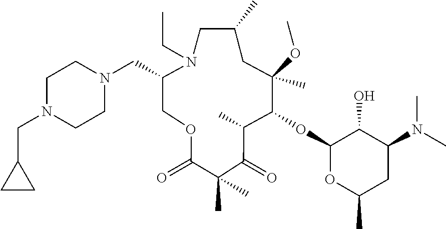 |
| 255 | 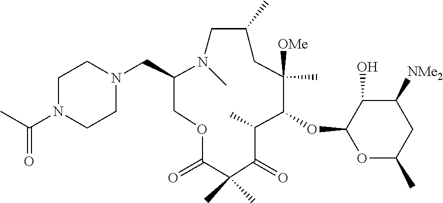 |
| 256 | 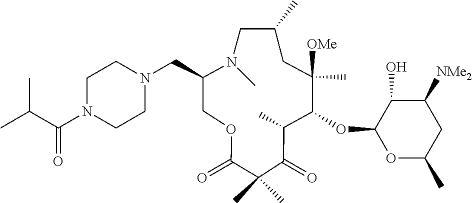 |
| 258 | 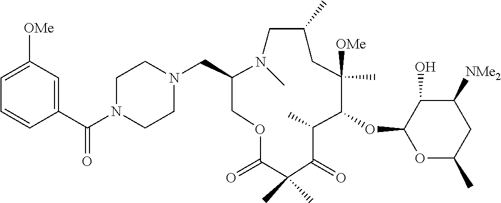 |
| 258 | 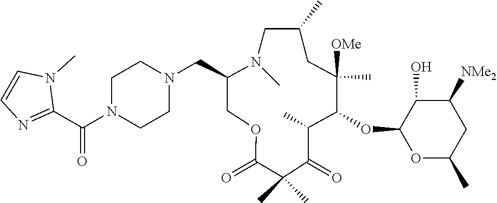 |
| 259 | 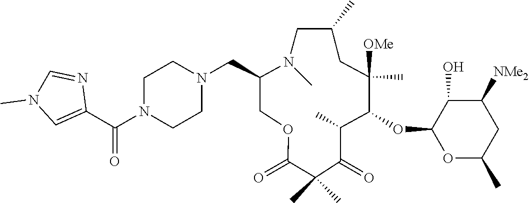 |
| 260 | 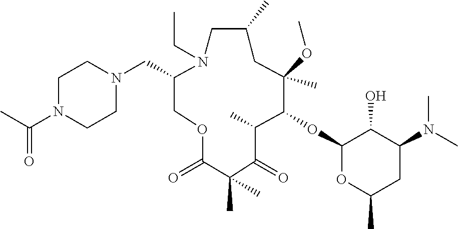 |
| 261 | 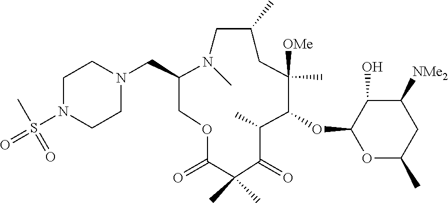 |
| 262 | 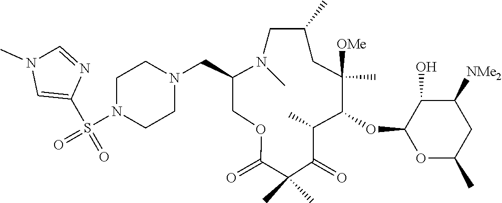 |
| 263 | 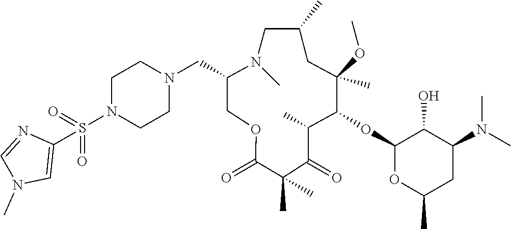 |
| 264 | 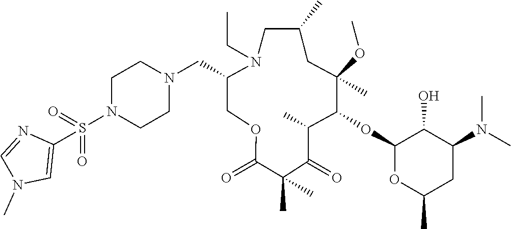 |
| 265 | 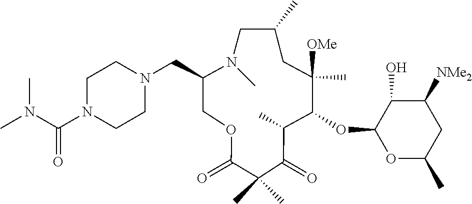 |
| 266 | 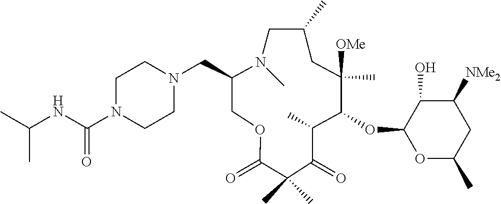 |
| 267 | 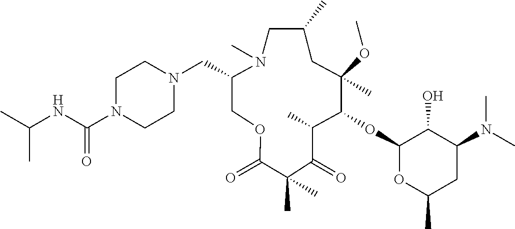 |
| 268 | 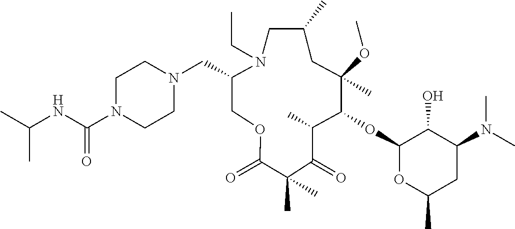 |
| 269 | 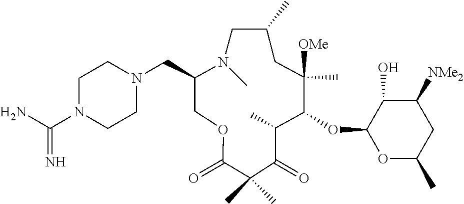 |
| 270 | 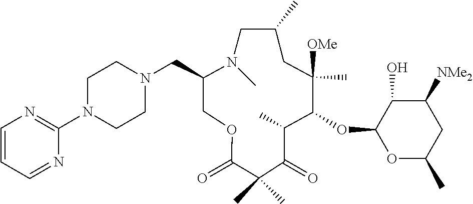 |
| 271 | 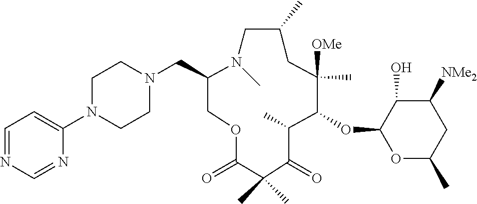 |
| 272 | 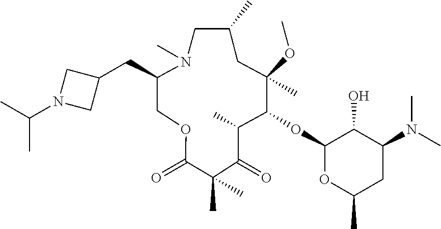 |
| 273 | 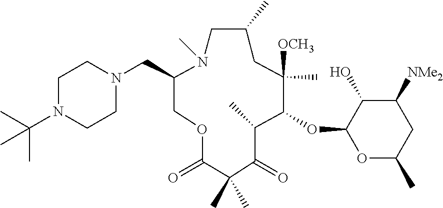 |
| 274 | 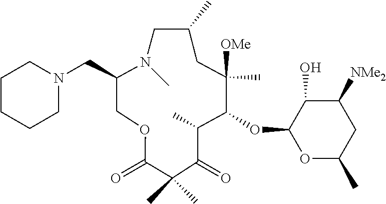 |
| 275 | 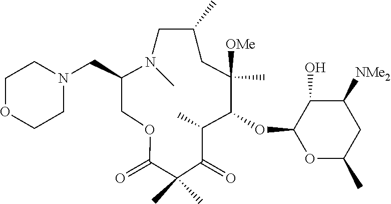 |
| 276 | 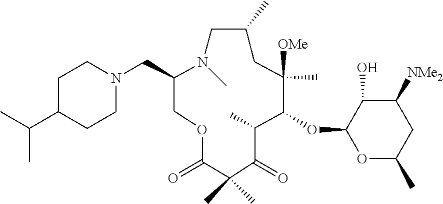 |
| 277 | 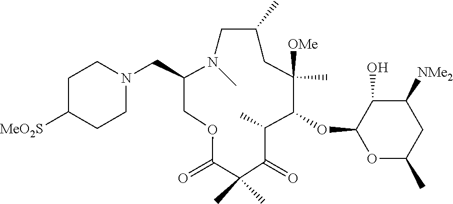 |
| 278 | 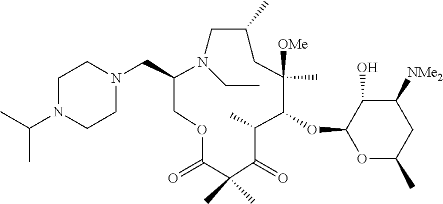 |
| 279 | 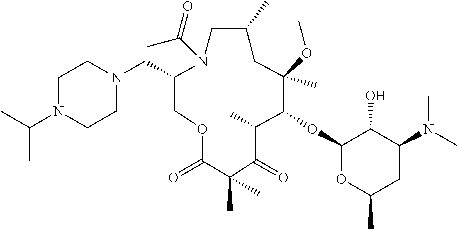 |
| 280 | 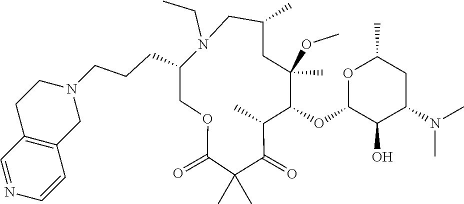 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCc2cnccc2C1] |
| 281 | 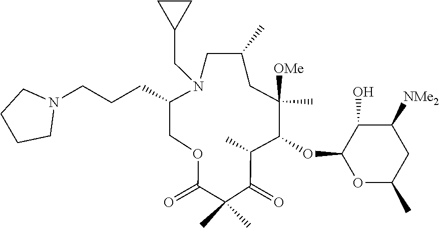 |
| 282 | 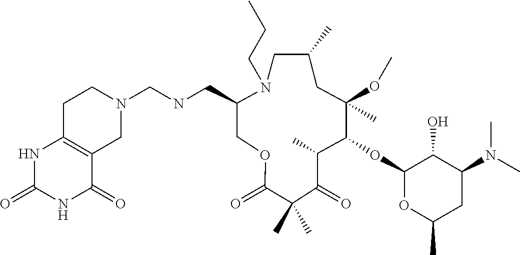 |

In these or other embodiments, the compound of formula I or a pharmaceutically acceptable salt thereof is administered to the subject alone or in any combination with an agent selected from the group consisting of aminoglycoside, potentiator, corrector, amplifier, and any combinations thereof.

### Processes for Preparing Compounds

Compounds disclosed herein can be prepared as described in the following paragraphs.

Compounds are prepared via two intermediates. The eastern half intermediate is a compound of formula P-1:

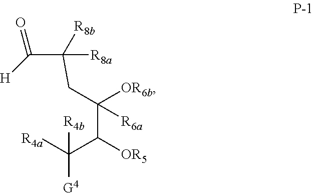

or salt thereof. In the compound of formula P-1, R³, R^{4a}, R^{4b}, R⁵, R^{6a}, R^{6b}, R^{8a}, and R^{8b} are as defined herein; and
G⁴ is of the formula:

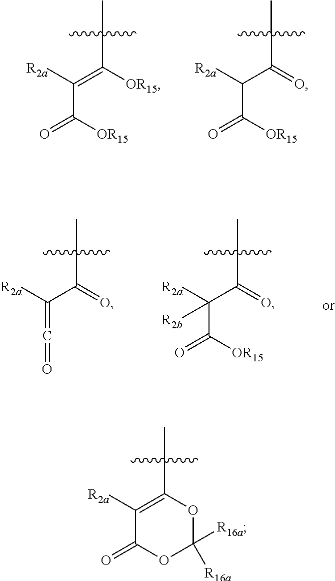

each instance of R¹⁵ is independently silyl, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R¹⁵ groups are joined to form an optionally substituted heterocyclyl or heteroaryl ring; and
each instance of R^{16a} is independently hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl.

The uncyclized eastern half intermediate is a compound of formula P-2:

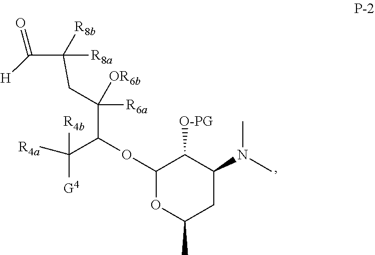

or salt thereof, wherein:
PG is a hydroxyl protecting group;
R₄ₐ, R_{4b}, R₅, R₆ₐ, R_{6b}, R₈ₐ, and R_{8b} are as defined herein;
G⁴ is of formula:

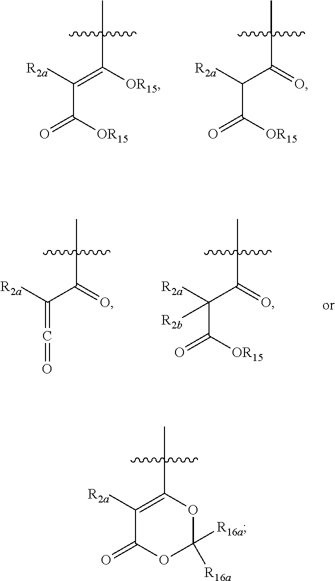

each instance of R¹⁵ is independently silyl, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R¹⁵ groups are joined to form an optionally substituted heterocyclyl or heteroaryl ring; and each instance of R^{16a} is independently hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl.

In some embodiments, —OPG is —OBz.

What is also disclosed is a compound of Formula P-3:

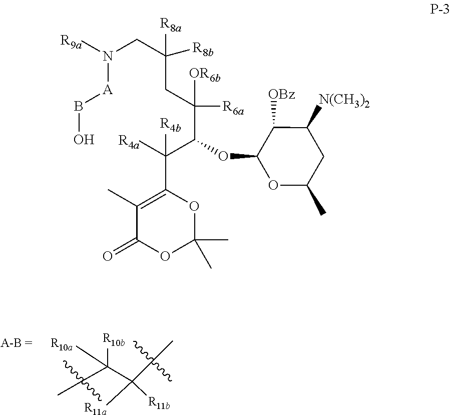

or salt thereof, wherein the variables are as defined herein.

### Coupling and Macrolactonization

In certain embodiments, compounds of the present disclosure are prepared by coupling a compound of Formula P1 (the eastern half) wherein R_{S} is a sugar residue

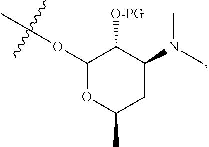

wherein PG is a hydroxyl protecting group and “ [symbol] ” indicates a point of attachment, and a compound of Formula P-4 (the western half) to provide an uncyclized compound precursor of Formula P-5 as depicted in the following Scheme.

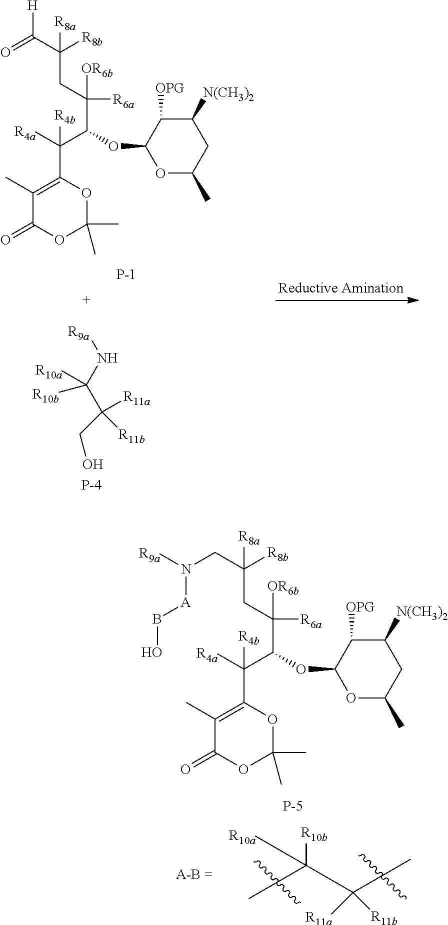

Formula P-5 is cyclized to give, after deprotection of the sugar residue a compound of Formula I as depicted in the following scheme.

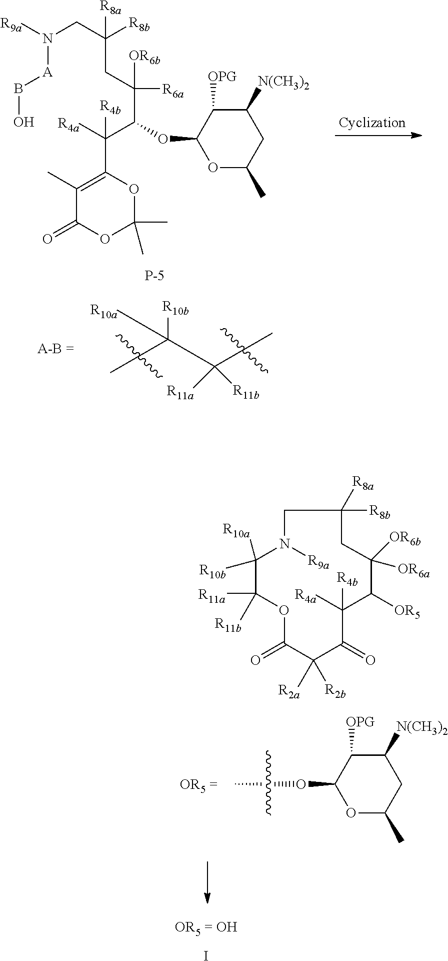

Alternatively, the compound precursor of Formula P-5 wherein R₉ₐ is hydrogen is cyclized to provide a compound of Formula I, which can undergo reductive amination to provide a compound of Formula I wherein R₉ₐ is other than H, as shown in the following Scheme.

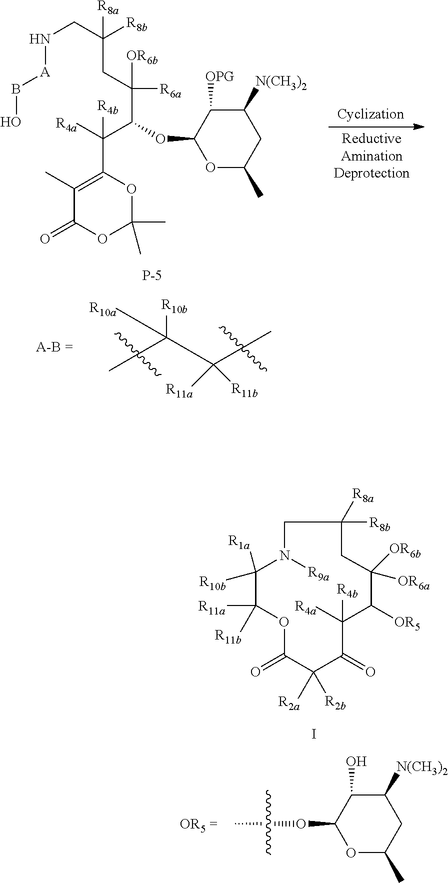

Late-stage installment of the R_{2b} group can be achieved via treatment of a compound of Formula P-6 prepared as provide above with a base and a suitable electrophile group (e.g., halogenating agent or R₂-LG, wherein LG is a leaving group) as depicted in the following Scheme. In this process, the sugar residue in P-6 is protected and R₉ₐ is H or alkyl.

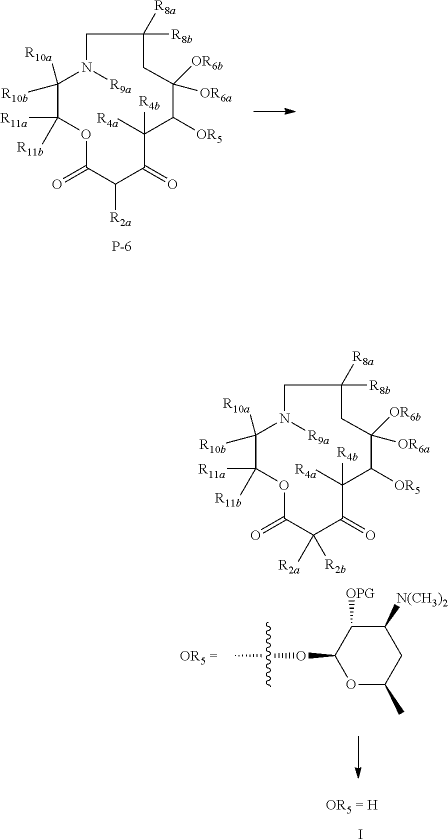

For all intermediates, the variables are as defined herein for a compound of Formula I.

Other variables depicted for intermediates and precursors are defined as follows:
LG is a leaving group;
G⁴ is of formula:

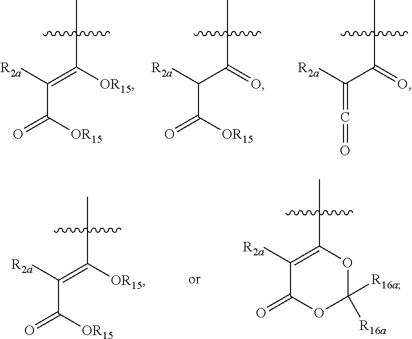

each instance of R¹⁵ is independently silyl, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R¹⁵ groups are joined to form an optionally substituted heterocyclyl or heteroaryl ring; and
each instance of R^{16a} is independently hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl.

As noted above, Rₛ is the sugar moiety

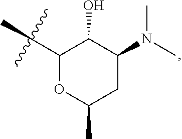 [SMILES: *C1O[C@H](C)C[C@H](N(C)C)[C@H]1O]

The sugar moiety is typically attached to the compound framework during synthesis of the eastern half, but may also be attached at other stages of the preparation. The sugar moiety may be attached by a chemical or enzymatic glycosylation reaction between the hydroxyl group at the C5 position and a glycosyl donor. In certain embodiments, the sugar moiety is attached to the compound framework as a thioglycoside. In certain embodiments, substituents of the sugar moiety are modified after the glycosylation of the compound or compound precursor (e.g., eastern half).

### Methods

In an aspect, what is provided herein is a method for treating a genetic disorder, comprising administering to a subject in need of such treatment a therapeutically effective amount of a compound of Formula I. What is also provided is a method of treating a genetic disorder characterized by a premature termination codon mutation, comprising administering to a subject in need thereof a therapeutically effective amount of to a subject in need of such treatment a therapeutically effective amount of a compound of Formula I.

A genetic disorder characterized by a premature termination codon mutation can be treated by inducing and/or promoting readthrough of the mutation in the complete but otherwise defective transcript (mRNA). That is, a genetic disorder characterized by a premature termination codon mutation can be treated by inducing and/or promoting suppression of the nonsense mutation (the premature termination codon mutation). Thus, as disclosed herein, a genetic disorder that can be treated according to the method disclosed and claimed herein is one that is responds to readthrough-inducing and/or promoting compounds.

Methods for identifying genetic disorders characterized by a premature termination codon mutation are available to the skilled practitioner, and may involve full or partial genome elucidation, genetic biomarker detection, phenotype classification and hereditary information analysis. These methods often result in pairs of mutant/wild type (WT) sequences. Pairs of WT sequences can be employed to identify whether the genetic disease is characterized by a premature termination codon mutation. Similarly, the ability to determine the ability of a compound or composition to induce or promote readthrough can also is also known in the art.

To that end, a plasmid comprising two reporter genes interrupted by a sequence of the mutated gene (the genetic disease-causing gene) is transected into a protein expression platform, either in full cells or in a cell-free systems, and the ratio between the expression level of the two genes in the presence of a tested compound is measured, typically in series of concentrations and duplications, and compared to the gene expression level ratio of the wild-type and/or to the expression level ratio measured in a control sample not containing the tested compound.

It is noted that the experimental model for readthrough activity (namely the nucleotide sequence of gene containing the premature stop-codon mutation) is a byproduct of the process of identifying a genetic disorder as associated with a premature stop-codon mutation and/or a protein truncation phenotype, and further noted that with the great advances in genomic data acquisition, this process is now well within the capability of the skilled prasctitioner.

Methodologies for testing readthrough of a premature termination codon mutation are known. Experimental methods are provided herein that are designed to guage the ability of compounds to induce or promote read through.

A number of in vitro methodologies can be used by the skilled practitioner to test the readthrough-inducing ability of compounds provided herein, as well as their safety as potential drugs.

Non-limiting examples of genetic diseases that are associated with the presence of at least one premature termination codon mutation or other nonsense mutations include cystic fibrosis (CF), muscular dystrophy (Duchenne (DMD), Becker (BMD), congenital, spinal muscular atrophy) ataxia-telangiectasia, mucopolysaccharidosis type I (Hurler syndrome), hemophilia (A & B), Usher syndrome (Retinitis pigmentosa, X-linked retinitis pigmentosa), Tay-Sachs, Factor VII deficiency, familial atrial fibrillation, Hailey disease, McArdle disease, mucopolysaccharidosis, nephropathic cystinosis, polycystic kidney disease, Rett syndrome, cystinosis, severe epidermolysis bullosa, Dravet syndrome, X-linked nephrogenic diabetes insipidus (XNDI), cancer, beta-thalassemia, epidermolysis bullosa (EB), familial adenomatous polyposis (FAP), and obesity.

In some embodiments, the genetic disorder is EB.

In some embodiments, the genetic disorder is severe EB.

In some embodiments, the genetic disorder is dystrophic epidermolysis bullosa (DEB), recessive dystrophic epidermolysis bullosa (RDEB), junctional epidermolysis bullosa (JEB), epidermolysis bullosa simplex (EBS), and/or kindler syndrome.

In some embodiments, the genetic disorder is RDEB, JEB, and/or FAP.

Additional genetic diseases that associated with the presence of at least one premature termination codon or other nonsense mutations are disclosed in, for example, “Suppression of nonsense mutations as a therapeutic approach to treat genetic diseases,” Kim M. Keeling, K. M Bedwell, D. M., Wiley Interdisciplinary Reviews: RNA, 2011, 2(6), p. 837-852; “Cancer syndromes and therapy by stop-codon readthrough,” Bordeira-Carrico, R. et al., Trends in Molecular Medicine, 2012, 18(11), p. 667-678, and references cited therein.

In an aspect what is provided is a compound or composition as disclosed herein for use in the treatment of a genetic disease associated with a premature termination codon mutation.

In another aspect, what is provided is a use of a compound or composition as disclosed herein in in the manufacture of a medicament for treating a genetic diseaase associated with a premature termination codon mutation. The genetic disease in this and other aspects and embodiments is selected from the group consisting of cystic fibrosis (CF), muscular dystrophy (Duchenne (DMD), Becker, congenital, spinal) ataxia-telangiectasia, Hurler syndrome, hemophilia (A & B), Usher syndrome (Retinitis pigmentosa, X-linked retinitis pigmentosa), Tay-Sachs, Factor VII deficiency, familial atrial fibrillation, Hailey disease, McArdle disease, mucopolysaccharidosis, nephropathic cystinosis, polycystic kidney disease, Rett syndrome, cystinosis, severe epidermolysis bullosa, Dravet syndrome, X-linked nephrogenic diabetes insipidus (XNDI), cancer, beta-thalassemia, EB, severe EB, DEB, RDEB, JEB, FAP, EBS, kindler syndrome, and obesity.

In another aspect, what is provided is a method of increasing the expression level of a gene having a premature termination codon mutation, the method comprising translating the gene into a protein in the presence of a compound or composition as disclosed herein in any of the respective embodiments and any combination thereof. In an embodiment what is provided is a compound or composition as disclosed herein for use in increasing the expression level of a gene having a premature termination codon mutation. In a further embodiment, what is a provided is the use of a compound or composition as disclosed in the manufacture of a medicament for increasing the expression level of a gene having a premature stop-codon mutation. According to these and other aspects and embodiments, the premature termination codon mutation has an RNA code selected from the group consisting of UGA, UAG and UAA.

According to these and other aspects and embodiments, the protein is translated in a cytoplasmic translation system. According to these and other aspects and embodiments, the compound or composition disclosed herein is used in a mutation suppression amount. According to these and other aspects and embodiments, an inhibition of translation IC₅₀ of the compound or composition in a eukaryotic cytoplasmic translation system is greater that an inhibition of translation IC₅₀ of the compound in a ribosomal translation system. According to these and other aspects and embodiments, an inhibition of translation IC₅₀ of the compound in a eukaryotic cytoplasmic translation system is greater that an inhibition of translation IC₅₀ of the compound in a prokaryotic translation system.

### Pharmaceutical Compositions and Administration

The present invention provides pharmaceutical compositions comprising a compound of the present invention and a pharmaceutically acceptable excipient. In certain embodiments, the compound of the present invention is provided in an effective amount in the pharmaceutical composition. In certain embodiments, the effective amount is a therapeutically effective amount. In certain embodiments, the effective amount is a prophylactically effective amount.

Pharmaceutically acceptable excipients include any and all solvents, diluents, or other liquid vehicles, dispersions, suspension aids, surface active agents, isotonic agents, thickening or emulsifying agents, preservatives, solid binders, lubricants and the like, as suited to the particular dosage form desired. General considerations in formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Pharmaceutical compositions described herein can be prepared by any method known in the art of pharmacology. In general, such preparatory methods include the steps of bringing the compound of the present invention (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, and/or mixtures thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof: malic acid and salts and hydrates thereof: phosphoric acid and salts and hydrates thereof: and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents such as Cremophor, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

A sterile injectable composition, e.g., a sterile injectable aqueous or oleaginous suspension, can be formulated according to techniques known in the art using suitable dispersing or wetting agents (such as Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are typically suppositories which can be prepared by mixing the conjugates of this invention with suitable non-irritating excipients or carriers such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the compounds presented herein may be dissolved or suspended in suitable liquids, such as fatty oils, liquid paraffin, or liquid polyethylene glycols. In addition, stabilizers may be added. All formulations for oral administration should be in dosages suitable for the chosen route of administration.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active aminoglycoside compounds doses.

The active ingredient can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active ingredient can be admixed with at least one inert diluent such as sucrose, lactose or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner Examples of embedding compositions which can be used include polymeric substances and waxes.

Dosage forms for topical and/or transdermal administration of a compound of this invention may include ointments, pastes, creams, lotions, gels, powders, solutions, sprays, inhalants and/or patches. Generally, the active ingredient is admixed under sterile conditions with a pharmaceutically acceptable carrier and/or any needed preservatives and/or buffers as can be required. Additionally, the present invention contemplates the use of transdermal patches, which often have the added advantage of providing controlled delivery of an active ingredient to the body. Such dosage forms can be prepared, for example, by dissolving and/or dispensing the active ingredient in the proper medium. Alternatively, or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662.

Intradermal compositions can be administered by devices which limit the effective penetration length of a needle into the skin, such as those described in PCT publication WO 99/34850 and functional equivalents thereof. Jet injection devices which deliver liquid vaccines to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively, or additionally, conventional syringes can be used in the classical mantoux method of intradermal administration.

A pharmaceutical composition of the invention can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions of the invention formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface-active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition of the invention. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) and as much as 100% (w/w) of the active ingredient, and may comprise one or more of the additional ingredients described herein. A pharmaceutical composition of the invention can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease, disorder, or condition being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

To practice the method of this invention, the above-described compound or its pharmaceutical composition can be administered intravenously, intravitreally, intradermally, transdermally, intrathecally, intraarterially, intraperitoneally, intranasally, intravaginally, intrarectally, intraosseously, periprosthetically, topically, intramuscularly, subcutaneously, mucosally, intraosseosly, periprosthetically, in utero, orally, topically, locally, via inhalation (e.g., aerosol inhalation), by injection, by infusion, by continuous infusion, by localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 2003, incorporated herein by reference). In general, the most appropriate route of administration will depend upon a variety of factors including the nature of the agent (e.g., its stability in the environment of the gastrointestinal tract), and/or the condition of the subject (e.g., whether the subject is able to tolerate oral administration).

In certain embodiments, the pharmaceutical composition and/or additional agent is formulated to be administered via an alimentary route. Alimentary routes include all possible routes of administration in which the composition is in direct contact with the alimentary tract.

Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsules, they may be compressed into tablets, or they may be incorporated directly with the food of the diet.

In further embodiments, a composition described herein may be administered via a parenteral route. As used herein, the term “parenteral” includes routes that bypass the alimentary tract. Specifically, the pharmaceutical compositions disclosed herein may be administered, for example but not limited to, intravenously, intradermally, intramuscularly, intraarterially, intrathecally, subcutaneous, or intraperitoneally

According to some embodiments, the administration is effected orally. For oral administration, the compounds presented herein can be formulated readily by combining the compounds with pharmaceutically acceptable carriers well known in the art. Such carriers enable the compounds presented herein to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

For buccal administration, the compositions may take the form of tablets or lozenges formulated in conventional manner.

For administration by inhalation, the compounds presented herein are conveniently delivered in the form of an aerosol spray presentation (which typically includes powdered, liquefied and/or gaseous carriers) from a pressurized pack or a nebulizer, with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compounds presented herein and a suitable powder base such as, but not limited to, lactose or starch.

For administration by injection, the compounds presented herein may be formulated in aqueous solutions, preferably in physiologically compatible buffers such as Hank's solution, Ringer's solution, or physiological saline buffer with or without organic solvents such as propylene glycol, polyethylene glycol.

Pharmaceutical compositions for topical administration may include the compositions formulated for a medicated application such as an ointment, paste, cream, or powder. Ointments include all oleaginous, adsorption, emulsion, and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the composition and provide for a homogenous mixture. Transdermal administration of the compositions may also comprise the use of a “patch.” For example, the patch may supply one or more compositions at a predetermined rate and in a continuous manner over a fixed period of time.

In certain embodiments, the compositions may be delivered by eye drops, intranasal sprays, inhalation, and/or other aerosol delivery vehicles. Methods for delivering compositions directly to the lungs via nasal aerosol sprays has been described in U.S. Pat. Nos. 5,756,353 and 5,804,212 (each specifically incorporated herein by reference in their entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts and could be employed to deliver the compositions described herein. Likewise, transmucosal drug delivery in the form of a polytetrafluoroethylene support matrix is described in U.S. Pat. No. 5,780,045 (specifically incorporated herein by reference in its entirety), and could be employed to deliver the compositions described herein.

It is further envisioned the compositions disclosed herein may be delivered via an aerosol. The term aerosol refers to a colloidal system of finely divided solid or liquid particles dispersed in a liquefied or pressurized gas propellant. The typical aerosol for inhalation consists of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

For transmucosal administration, penetrants are used in the formulation. Such penetrants are generally known in the art.

The compounds presented herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Alternatively, the compounds presented herein may be in powder form for constitution with a suitable vehicle, e.g., sterile, pyrogen-free water, before use.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the invention may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional therapeutically active agents. The compounds or compositions can be administered in combination with additional therapeutically active agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The compound or composition can be administered concurrently with, prior to, or subsequent to, one or more additional therapeutically active agents. In general, each agent will be administered at a dose and/or on a time schedule determined for that agent. In will further be appreciated that the additional therapeutically active agent utilized in this combination can be administered together in a single composition or administered separately in different compositions. The particular combination to employ in a regimen will take into account compatibility of the inventive compound with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually. Additional therapeutically active agents include antibiotic agents, e.g., antibiotics useful for treating tuberculosis. Exemplary antibiotics include, but are not limited to, isoniazid, rifampin, pyrazinamide, ethambutol, and streptomycin.

Also encompassed by the invention are kits (e.g., pharmaceutical packs). The kits provided may comprise an inventive pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or compound. In some embodiments, the inventive pharmaceutical composition or compound provided in the container and the second container are combined to form one-unit dosage form.

### EXAMPLES

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

Table 1 lists intermediates that were used in the preparation of example compounds.

TABLE 1

| Aminoalcohol | | |
|---|---|---|
| Number | Structure | Source |
| I1 | 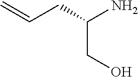 | Commercial |
| I2 | 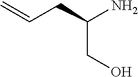 | Commercial |
| I3 | 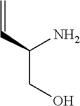 | Commercial |
| I4 | 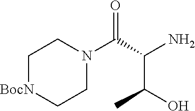 [SMILES: C[C@H](O)[C@@H](N)C(=O)N1CCN(C(=O)OC(C)(C)C)CC1] | Methods of Intermediate Scheme 1 |
| I5 | 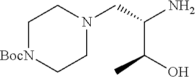 [SMILES: C[C@H](O)[C@@H](N)CN1CCN(C(=O)OC(C)(C)C)CC1] | Methods of Intermediate Scheme 1 |
| I6 | 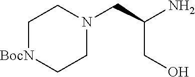 [SMILES: CC(C)(C)OC(=O)N1CCN(C[C@@H](N)CO)CC1] | Methods of Intermediate Scheme 1 |
| I7 | 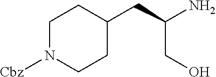 [SMILES: N[C@@H](CO)CC1CCN(C(=O)OCc2ccccc2)CC1] | Methods of Intermediate Scheme 2 |
| I8 | 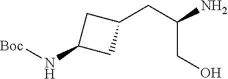 [SMILES: CC(C)(C)OC(=O)N[C@H]1C[C@H](C[C@@H](N)CO)C1] | Methods of Intermediate Scheme 3 |
| I9 | 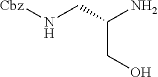 [SMILES: N[C@H](CO)CNC(=O)OCc1ccccc1] | Commercial |
| I10 | 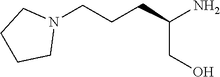 [SMILES: N[C@@H](CO)CCCN1CCCC1] | Methods of Intermediate Scheme 4 |
| I11 | 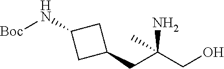 [SMILES: CC(C)(C)OC(=O)N[C@H]1C[C@H](C[C@@](C)(N)CO)C1] | Methods of Intermediate Scheme 5 |
| I12 | 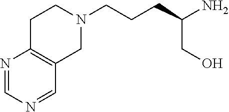 [SMILES: N[C@@H](CO)CCCN1CCc2ncncc2C1] | Methods of Intermediate Scheme 6 |
| I13 | 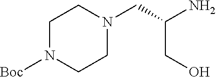 [SMILES: CC(C)(C)OC(=O)N1CCN(C[C@H](N)CO)CC1] | Methods of Intermediate Scheme 1 |
| I14 | 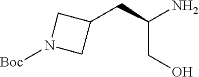 [SMILES: CC(C)(C)OC(=O)N1CC(C[C@@H](N)CO)C1] | Methods of Intermediate Scheme 5 |
| I15 | 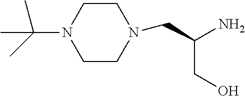 | Methods of Intermediate Scheme 7 |
| I16 | 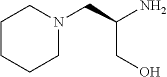 [SMILES: N[C@@H](CO)CN1CCCCC1] | Methods of Intermediate Scheme 1 |
| I17 | 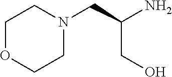 [SMILES: N[C@@H](CO)CN1CCOCC1] | Methods of Intermediate Scheme 1 |
| I18 | 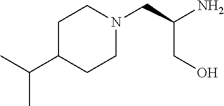 | Methods of Intermediate Scheme 1 |
| I19 | 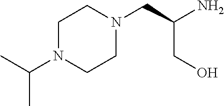 | Methods of Intermediate Scheme 1 |

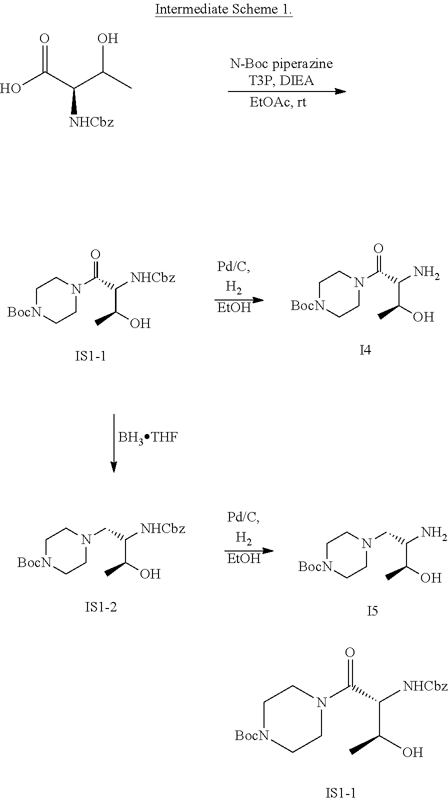

tert-Butyl 4-(((benzyloxy)carbonyl)-D-threonyl)piperazine-1-carboxylate (IS1-1)

To a solution of (2R,3S)-2-{[(benzyloxy)carbonyl]amino}-3-hydroxybutanoic acid (3 g, 11.8 mmol) in EtOAc (50 mL) was added DIEA (1.8 mL, 10.3 mmol) and tert-butyl piperazine (2 g, 10.7 mmol). 1-Propanephosphonic anhydride (T3P®) (8.63 g of a 50% w/w soln in dichloromethane) was added by pipet with stirring over 2 minutes (mins) and the reaction mixture was stirred for 6.5 hours (h). The reaction mixture was diluted with EtOAc (40 mL) and washed sequentially with 1 M HCl aqueous (aq.) (120 mL), water (50 mL), sat. NaHCO₃ (80 mL), dried over Na2SO4, filtered, concentrated, and re-concentrated from dichloromethane/MBTE to give an off-white foam. The crude product was purified by silica gel chromatography eluting with EtOAc/dichloromethane (0-70% gradient) to yield the title compound (1.70 g). 1H NMR (400 MHz, Chloroform-d) δ 7.38-7.25 (m, 5H), 6.03 (d, 1H), 5.09 (s, 2H), 4.49 (d, 1H), 4.15-3.99 (m, 2H), 3.77-3.58 (m, 2H), 3.45 (tt, 4H), 3.36-3.21 (m, 2H), 1.46 (s, 9H), 1.15 (d, 3H).

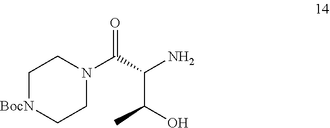 [SMILES: C[C@H](O)[C@@H](N)C(=O)N1CCN(C(=O)OC(C)(C)C)CC1]

tert-Butyl 4-(D-threonyl)piperazine-1-carboxylate (I4)

IS1-1 (766 mg, 1.03 mmol) was dissolved in absolute EtOH (12 mL) and the reaction mixture was evacuated and back-filled with nitrogen (3 times). 5% Pd/C (109 mg, 0.05 mmol) was added and the reaction mixture was evacuated and back-filled with nitrogen (3 times). The reaction mixture was then evacuated and back-filled with hydrogen (3 times) and stirred at room temperature (rt) under a hydrogen atmosphere (balloon) for 1.5 h. The reaction mixture was evacuated and back-filled with nitrogen (5 times). Diatomaceous earth (Celite®) was added to reaction mixture it was stirred for 5 min, and filtered through a MeOH wetted pad of Celite® rinsed with MeOH and concentrated. The crude material was dissolved in dichloromethane and filtered through a syringe filter to deliver the crude product. MS (ESI+) m/z: 288.03 [M+H]+, 1H NMR (400 MHz, Chloroform-d) δ 3.86 (td, 1H), 3.79 (s, 10H), 1.47 (s, 9H), 1.18 (d, 3H).

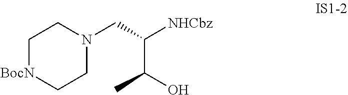 [SMILES: C[C@H](O)[C@H](CN1CCN(C(=O)OC(C)(C)C)CC1)NC(=O)OCc1ccccc1]

tert-Butyl 4-((2S,3S)-2-(((benzyloxy)carbonyl)amino)-3-hydroxybutyl)piperazine-1-carboxylate (IS1-2)

In an oven-dried 3-necked flask fitted with a reflux condenser, IS1-1 (1.25 g, 2.96 mmol) was dissolved in dry THE (29 mL, 0.1 M) and cooled to 0° C. under nitrogen. 1 M Borane·THF complex (8.8 mL, 8.8 mmol) was added dropwise over 11.5 min, keeping the temperature below 3.5° C. A slight evolution of gas was observed. The reaction mixture was stirred for 6 min, the ice-bath was removed, and then the reaction mixture was allowed to warm to 16.5° C. and then heated to 65° C. for 2 h. The reaction mixture was cooled in an ice-bath and slowly quenched by the addition of MeOH (7 mL). The reaction mixture was diluted with additional MeOH and concentrated (3 times). The residue was dissolved in MeOH (50 mL), heated to a gentle reflux for approximately 1 h and concentrated. The crude product was purified by silica gel chromatography eluting with 20% MeOH in dichloromethane+0.5% NH4OH/CH2Cl2 (0-60% gradient) to yield a white foam (766 mg). MS (ESI+) m/z: 408.13 [M+H]+, 1H NMR (400 MHz, Chloroform-d) δ 7.44-7.29 (m, 5H), 5.24 (d, 1H), 5.11 (s, 2H), 4.05 (qd, 1H), 3.65 (d, 1H), 3.48-3.31 (m, 4H), 2.71 (dd, 1H), 2.56-2.46 (m, 3H), 2.42 (dt, 2H), 1.45 (s, 9H), 1.18 (d, 3H).

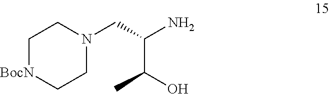 [SMILES: C[C@H](O)[C@@H](N)CN1CCN(C(=O)OC(C)(C)C)CC1]

tert-Butyl 4-((2S,3S)-2-amino-3-hydroxybutyl)piperazine-1-carboxylate (I5)

IS1-2 (766 mg, 1.87 mmol) was dissolved in absolute EtOH (20 mL) and the reaction mixture was evacuated and back-filled with nitrogen (3 times). 5% Pd/C (200 mg, 0.94) was added and the reaction mixture was evacuated and back-filled with nitrogen (3 times). The reaction mixture was evacuated and back-filled with hydrogen (3 times) and stirred at rt under a hydrogen atmosphere (balloon) for 1.5 h and heated to 45° C. for 1 h. The reaction mixture was cooled to rt, evacuated and back-filled with nitrogen (5 times). Celite® was added to reaction mixture it was stirred for 5 min, and filtered through a MeOH wetted pad of Celite® rinsed with MeOH and concentrated to yield the crude product. MS (ESI+) m/z: 274.08 [M+H]+, 1H NMR (400 MHz, Chloroform-d) δ 7.41-7.33 (m, OH), 3.56 (qd, 1H), 3.50-3.34 (m, 4H), 2.93-2.78 (m, 1H), 2.48 (d, 3H), 2.43-2.28 (m, 3H), 1.45 (s, 10H), 1.17 (d, 3H).

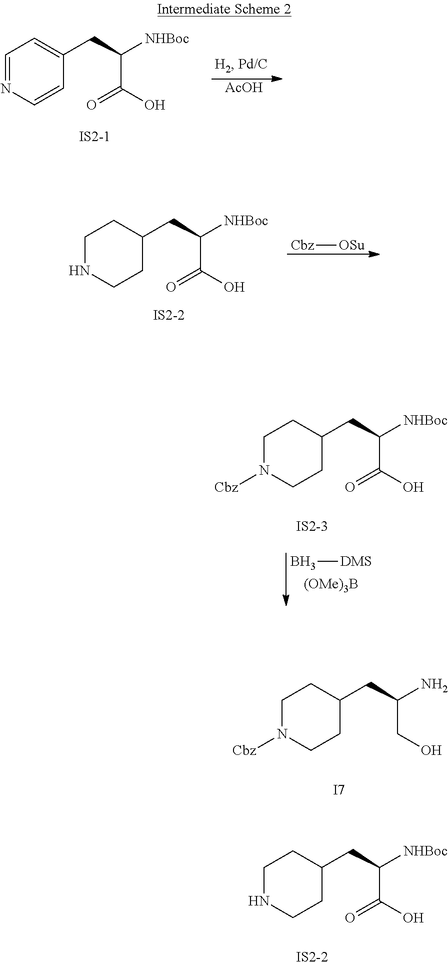

(R)-2-((tert-butoxycarbonyl)amino)-3-(piperidin-4-yl)propanoic acid (IS2-2)

An oven-dried flask was evacuated and back-filled with nitrogen (2 times) before cooling to rt. 10% Pd/C (50% wet, 7.96 g, 3.74 mmol) was added to the flask which was evacuated and back-filled with nitrogen (2 times). Glacial acetic acid (32 mL) was added to the reaction which was evacuated and back-filled with nitrogen (2 times). N-Boc-D-pyridylalanine (5 g, 18.7 mmol) was added followed by glacial acetic acid (5 mL). The reaction was evacuated and back-filled with nitrogen (2 times) and was then evacuated and back-filled with hydrogen (4 times). The reaction mixture was heated to 60° C. and was stirred under a hydrogen balloon for 15 h. The reaction mixture was cooled to rt and was evacuated and back-flushed with nitrogen (4 times). Celite® was added, and the reaction mixture was stirred for approximately 15 min and was then filtered through a pad of Celite® while rinsing with MeOH. The reaction mixture was concentrated and then re-concentrated from MTBE to give a clear gum. The material was used without further purification. MS (ESI+) m/z: 273.07 [M+H]+.

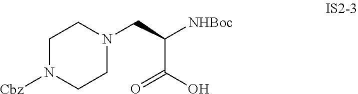 [SMILES: CC(C)(C)OC(=O)N[C@H](CN1CCN(C(=O)OCc2ccccc2)CC1)C(=O)O]

(R)-3-(1-((benzyloxy)carbonyl)piperidin-4-yl)-2-((tert-butoxycarbonyl)amino)propanoic acid (IS2-3)

Crude IS2-2 (4.15 g, 15.2 mmol) was dissolved in THE (30 mL) to which sat. aq. NaHCO3 (20 mL) was added. The reaction mixture was cooled to 0° C., and N-(benzyloxycarbonyloxy)succinimide (4.16 g, 16.7 mmol) was added. The reaction mixture was stirred for 11 min, the ice-bath was removed, and the reaction mixture was stirred at room temperature. Upon completion, the reaction mixture was cooled in an ice-bath, and N HCl (approximately 50 mL) was added slowly until bubbling ceased and the solution was pH 2-3. The reaction mixture was extracted with MTBE (25 mL×3). The combined extracts were washed with 1N HCl (20 mL×2), water (40 mL), and brine (40 mL) and were dried over MgSO4, were filtered, and were concentrated. The material was purified on 80 g silica gel (dichloromethane/EtOAc+1% AcOH Gradient: 0-100%) to give the title compound (2.3 g, 37%, 2 steps). MS (ESI+) m/z: 429.09 [M+Na]+. 1H NMR (400 MHz, Chloroform-d) δ 7.42-7.28 (m, 5H), 5.14 (s, 2H), 4.95 (d, 1H), 4.44-4.32 (m, 1H), 4.30-4.07 (m, 2H), 2.89-2.66 (m, 2H), 1.91-1.51 (m, 5H), 1.46 (s, 9H), 1.27-1.06 (m, 2H).

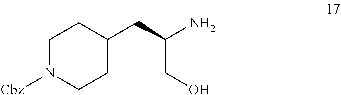 [SMILES: N[C@@H](CO)CC1CCN(C(=O)OCc2ccccc2)CC1]

Benzyl (R)-4-(2-amino-3-hydroxypropyl)piperidine-1-carboxylate (I7)

In an oven-dried flask, crude IS2-3 (2.3 g, 5.65 mmol) was concentrated from dry toluene (10 mL), was dissolved in dry THE (12 mL) under N2, and was cooled to 0° C. Trimethyl borate (1.37 mL, 12.4 mmol) was added, and the reaction mixture was stirred for approximately 7 min. Borane dimethylsulfide complex (0.80 mL, 8.47 mmol) was added dropwise by syringe over approximately 4 minutes such that the temperature did not exceed 3° C. The reaction mixture was stirred for 10 min, the ice-bath was removed, and the reaction mixture was stirred at rt for 5.5 h. The reaction mixture was cooled to 0° C. and additional trimethylborate (0.7 mL) and borane dimethylsulfide (0.4 mL) were added; the reaction mixture was allowed to slowly warm to rt over 1.5 h. The reaction mixture was cooled to 0° C. and methanol (10 mL) was added dropwise over 15 min, keeping the temperature below 10° C. The ice-bath was removed, and the reaction mixture was stirred for 30 min and was concentrated. The resulting clear oil was re-dissolved in methanol (approximately 50 mL) and was concentrated (2 times) before being placed on the high vac for approximately 20 min. The residue was partitioned between 1 N HCl (30 mL) and MTBE (25 mL). The aqueous layer was extracted with MTBE (25 mL×2). The aqueous layer was basified with sat, aq. NaHCO₃ (pH approximately 8.5) and was extracted with EtOAc (20 mL×3). The combined organic layers were dried over Na2SO4, were filtered, and were concentrated. MS (ESI+) m/z: 293.01 [M+H]+. 1H NMR (400 MHz, Chloroform-d) δ 7.31-7.24 (m, 2H), 7.24-7.20 (m, 1H), 7.20-7.11 (m, 1H), 7.11-7.01 (m, 1H), 5.03 (s, 2H), 4.18-3.96 (m, 2H), 3.47 (dd, 1H), 3.16 (ddd, 1H), 2.84 (tt, 1H), 2.78-2.57 (m, 2H), 1.67-1.41 (m, 3H), 1.27-0.88 (m, 4H).

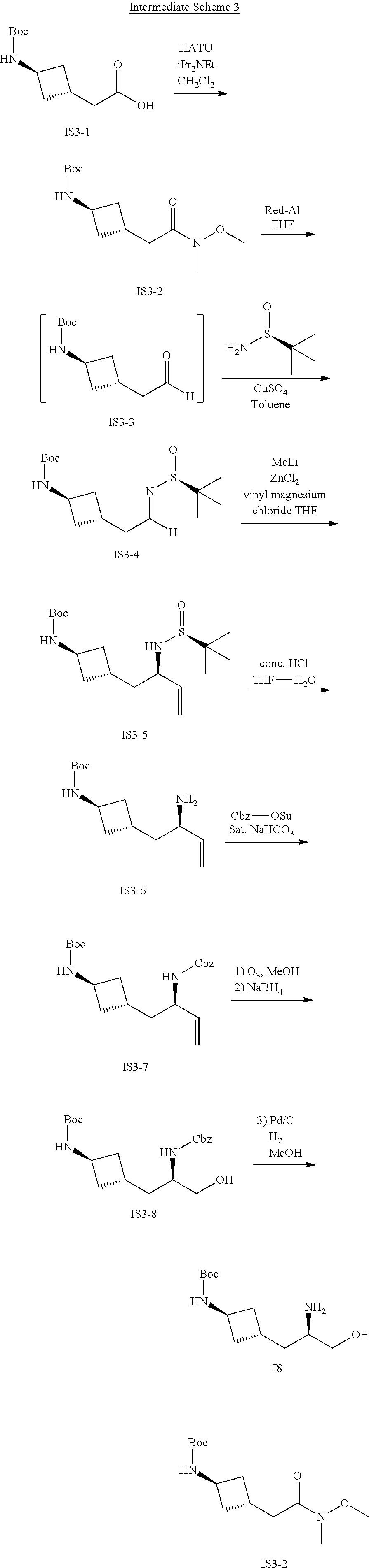

tert-Butyl ((1r,3r)-3-(2-(methoxy(methyl)amino)-2-oxoethyl)cyclobutyl)carbamate (IS3-2)

To a solution of 2-((1R,3R)-3-((tert-butoxycarbonyl)amino)cyclobutyl)acetic acid (IS3-1, 1.1 g, 4.8 mmol) in dichloromethane (20 mL) was added methyoxyl(methyl)amine hydrochloride (0.70 g, 7.2 mmol), N,N-diisopropylethylamine (4.15 mL, 24.0 mmol), and 1-[Bis(dimethylamino) methylene]-1H-1,2,3-triazolo[4,5-b]pyridinium 3-oxid hexafluorophosphate (HATU) (2.73 g, 7.2 mmol). The reaction mixture was stirred at room temperature for 16 h. The reaction mixture was poured into 1 M NaOH and was stirred vigorously for 10 min. The organic layer was separated and was washed with 2N HCl (2 times), water (1 time), and brine (1 time). The organic layer was dried over sodium sulfate and was concentrated in vacuo. The crude material was purified by column chromatography (80 g silica gel column, 0-50% EtOAC/Hex) to give the title compound as a white powder (1.19 g, 4.4 mmol, 92%). MS (ESI+) m/z: [M+Na]+295.2.

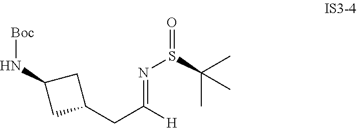

tert-Butyl ((1R,3r)-3-((E)-2-(((R)-tert-butylsulfinyl)imino)propyl)cyclobutyl)carbamate (IS3-4)

IS3-2 (1.19 g, 4.4 mmol) in THF (20 mL) was cooled to −40° C., and Red-Al (1.83 mL, 70 wt % in toluene, 5.7 mmol) was added. The reaction mixture was allowed to warm to room temperature and was stirred for 16 h. Ethyl acetate and sat. aq. potassium sodium tartrate (Rochelle salt) was added, and the mixture was stirred vigorously for 2 h. The organic layer was separated and was washed with brine (1 time), was dried over sodium sulfate, was filtered, and was concentrated to give aldehyde IS3-3 as a clear oil. IS3-3 (0.96 g, 4.5 mmol) was dissolved in toluene (9 mL), (S)-2-methylpropane-2-sulfinamide (0.545 g, 4.5 mmol) followed by copper(II) sulfate (2.15 g, 13.5 mmol) were added. The reaction mixture was stirred at rt for 18 h and was filtered through Celite®, eluting with ethyl acetate. The filtrate was concentrated and was purified by column chromatography on silica gel (24 g, 0-70% EtOAc/Hex) to give the title compound (0.53 g, 1.67 mmol, 37%). 1H NMR (400 MHz, Chloroform-d) δ 7.99 (t, 1H), 4.71 (s, 1H), 4.24 (s, 1H), 2.70 (dd, 2H), 2.65-2.51 (m, 1H), 2.23-1.98 (m, 4H), 1.43 (d, 10H), 1.18 (d, 9H).

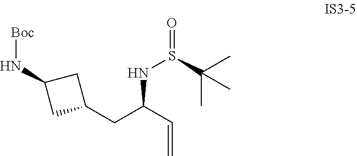 [SMILES: C=C[C@@H](C[C@H]1C[C@H](NC(=O)OC(C)(C)C)C1)N[S@@](=O)C(C)(C)C]

tert-Butyl ((1S,3r)-3-((R)-2-(((S)-tert-butylsulfinyl)amino)but-3-en-1-yl)cyclobutyl)carbamate (IS3-5)

A solution of ZnCl2 (2.63 mL, 1.9 M in MeTHF, 5.01 mmol) was added to dry THE (3.34 mL) and cooled to −78° C. A solution of methyllithium (3.22 mL, 3.1M in DME, 10 mmol) was added slowly, keeping the internal reaction temperature below −65° C. The mixture was stirred for 10 min, and a solution of vinylmagnesium chloride (3.22 mL, 1.6 M in THF, 3.13 mmol) was added slowly, keeping the internal reaction temperature below −65° C. The mixture was stirred for 5 min. A solution of IS3-4 (0.53 g, 1.67 mmol) in THE (1 mL) was added dropwise, and the reaction mixture was stirred for 30 min. Acetic acid (0.5 mL) was added slowly, the bath was removed, and the reaction mixture was allowed to warm to rt over 20 min. Half saturated (sat.) aq NH4Cl was added followed by MTBE. The layers were separated, the aqueous layer was extracted with MBTE (2 times), and the combined extracts were dried over Na2SO4, were filtered, and were concentrated. The crude material was purified by column chromatography (12 g silica gel column, 0-50% EtOAC/Hex) to give the title compound as a white powder (0.366 g, 1.06 mmol, 64%). 1H NMR (400 MHz, Chloroform-d) δ 5.61 (dddd, 1H), 5.22-5.08 (m, 2H), 4.73 (s, 1H), 4.10 (s, 1H), 3.76-3.67 (m, 1H), 3.06 (d, 1H), 2.41-2.26 (m, 1H), 2.13-1.92 (m, 4H), 1.74 (td, 2H), 1.42 (s, 9H), 1.19 (d, 9H).

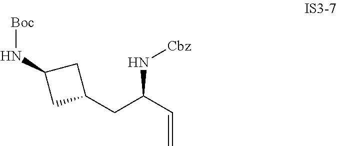 [SMILES: C=C[C@@H](C[C@H]1C[C@H](NC(=O)OC(C)(C)C)C1)NC(=O)OCc1ccccc1]

Benzyl ((R)-1-((1r,3S)-3-((tert-butoxycarbonyl)amino)cyclobutyl)but-3-en-2-yl)carbamate (IS3-7)

Concentrated HCl (0.1 mL, 1.27 mmol) was added to a solution of IS3-5 (0.366 g, 1.06 mmol) in THF/water (5:2, 2.8 mL), and the reaction mixture was stirred at room temperature for 18 h. Sat. aq. NaHCO₃ (3 mL) was added followed by N-(benzyloxycarbonyloxy)succinimide (0.276 g, 1.11 mmol). The reaction mixture was stirred at room temperature for 1 hour and was extracted with EtOAc (2 times). The combined extracts were washed with brine, were dried over sodium sulfate, were filtered, and were concentrated in vacuo. The material was purified by column chromatography on silica gel (12 g, 0-70% EtOAc/Hex) to give the title compound as a white powder (0.31 g, 0.83 mmol, 78%). MS (ESI+) m/z: 397.31 [M+Na]+; 1H NMR (400 MHz, Chloroform-d) δ 7.40-7.30 (m, 5H), 5.73 (ddd, 1H), 5.18-5.04 (m, 4H), 4.63 (d, 2H), 4.12 (s, 2H), 2.28 (s, 1H), 2.06 (s, 2H), 1.97 (s, 2H), 1.74-1.59 (m, 2H), 1.43 (s, 9H).

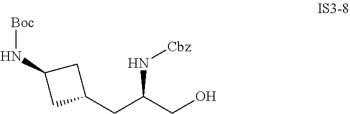 [SMILES: CC(C)(C)OC(=O)N[C@H]1C[C@H](C[C@H](CO)NC(=O)OCc2ccccc2)C1]

Benzyl ((R)-1-((1r,3S)-3-((tert-butoxycarbonyl)amino)cyclobutyl)-3-hydroxypropan-2-yl)carbamate (IS3-8)

IS3-7 (0.31 g, 0.827 mmol) was dissolved in methanol (16.5 mL) and was cooled to −78° C. A stream of ozone (7 PSI, 2 LPM) was bubbled through the reaction mixture for 8 min, at which point a slight blue coloration was observed. The ozone stream was removed, and nitrogen was then bubbled through the solution for 5 min (blue color disappeared). Sodium borohydride (77.1 mg, 2.04 mmol) was added, and the reaction mixture was removed from the bath and was allowed to warm to room temperature for 30 min. The reaction was quenched with sat. aq NH4C₁ and was extracted with dichloromethane (3 times). The combined extracts were dried over Na2SO₄, were filtered, and were concentrated in vacuo. The material was purified with column chromatography on silica gel (12 g, 0-70% EtOAc/Hex) to give the title compound as a white foam (0.265 g, 0.7 mmol, 85%). MS (ESI+) m/z: 401.09 [M+Na]+; 1H NMR (400 MHz, Chloroform-d) δ 7.44-7.30 (m, 5H), 5.09 (s, 2H), 4.82 (s, 1H), 4.69 (s, 1H), 4.22-4.09 (m, 1H), 3.67 (s, 2H), 3.55 (s, 1H), 2.28 (s, 1H), 2.06 (d, 2H), 1.99 (s, 3H), 1.74-1.60 (m, 2H), 1.43 (s, 9H).

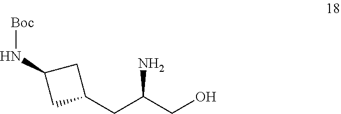 [SMILES: CC(C)(C)OC(=O)N[C@H]1C[C@H](C[C@@H](N)CO)C1]

tert-Butyl ((1S,3r)-3-((R)-2-amino-3-hydroxypropyl)cyclobutyl)carbamate (I8)

A solution of IS3-8 (265 mg, 0.7 mmol) was dissolved in methanol (3 mL) and Pd/C was added (74.3 mg, 5 wt % on charcoal, 0.5 mol %). A balloon of hydrogen was bubbled through the reaction mixture for 0.5 hr. The reaction mixture was filtered through Celite®, eluting with methanol, and the filtrate was concentrated in vacuo to give 18 as a clear oil (171 mg, 0.7 mmol, 100%). MS (ESI+) m/z: 245.08 [M+Na]+; 1H NMR (400 MHz, Methanol-d4) δ 4.13-4.01 (m, 1H), 3.64 (dd, 1H), 3.42 (dd, 1H), 2.97 (dt, 1H), 2.39-2.23 (m, 1H), 2.16-1.95 (m, 4H), 1.69 (ddt, 2H), 1.43 (s, 9H).

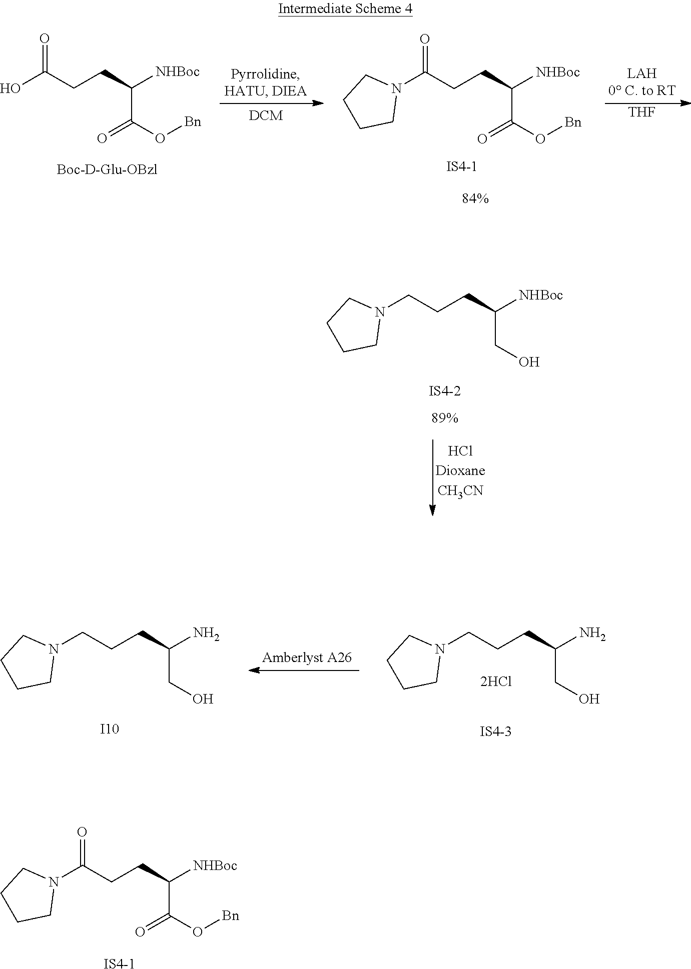

benzyl (R)-2-((tert-butoxycarbonyl)amino)-5-oxo-5-(pyrrolidin-1-yl)pentanoate (IS4-1): To a solution of Boc-D-glutamic acid 1-benzyl ester (20.0 g, 59.2 mmol, 1 equiv.) in dichloromethane (118 mL, 0.5M) was added pyrrolidine (7.3 mL, 76.9 mmol, 1.5 equiv.), Hunig's base (30.7 mL, 177 mmol, 3 equiv.), and HATU (29.2 g, 76.9 mmol, 1.3 equiv.). The reaction mixture was stirred at room temperature overnight, at which point UPLC showed complete conversion of the starting material to the desired mass. The reaction mixture was poured into 1 M NaOH (300 mL) and was stirred vigorously for 10 min. The organic layer was separated and was further washed with 2N HCl (2×300 mL), water (1×300 mL), and brine (1×300 mL). The washed solution was dried over magnesium sulfate and was concentrated in vacuo. The crude material was purified by silica gel chromatography (ISCO 330 g column), eluting with a gradient of 0 to 50% EtOAc in Hexanes. This gave 19.5 g (84%) of compound IS4-1. ¹H NMR (400 MHz, Chloroform-d) δ 7.41-7.28 (m, 5H), 5.51 (d, 1H), 5.27-5.05 (m, 2H), 4.40-4.26 (m, 1H), 3.43 (t, 2H), 3.27 (t, 2H), 2.38-2.12 (m, 3H), 2.12-1.95 (m, 1H), 1.95-1.75 (m, 4H), 1.42 (s, 9H).

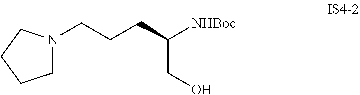 [SMILES: CC(C)(C)OC(=O)N[C@@H](CO)CCCN1CCCC1]

tert-butyl (R)-(1-hydroxy-5-(pyrrolidin-1-yl)pentan-2-yl)carbamate (IS4-2)

A solution of compound IS4-1 (4.5 g, 11.5 mmol, 1.0 equiv.) in anhydrous tetrahydrofuran (100 mL) was added drop-wise to a suspension of LiAlH₄ (1.0 M solution in THF, 48.3 mL, 48.3 mmol, 4.2 equiv.) in anhydrous THE (57.4 mL) at 0° C. Upon complete addition, the reaction mixture was allowed to stir at 0° C. for 0.5 h and was then warmed to rt for 1 h. The mixture was quenched by the cautious addition of water (2 mL), 3N NaOH solution (2.5 mL), and then water (5.5 mL). This mixture was dried with MgSO₄, and the precipitate was removed by filtration. The precipitate was washed with EtOAc, and the combined filtrate was concentrated under reduced pressure. The material was purified by silica gel chromatography, eluting with a gradient of 0 to 20% MeOH in DCM containing 0.5% NH4OH). This gave 2.81 g (89%) of compound IS4-2. ¹H NMR (400 MHz, Chloroform-d) δ 5.29 (s, 1H), 3.61-3.50 (m, 3H), 3.45 (t, 2H), 3.39 (t, 2H), 2.44-2.24 (m, 2H), 2.01-1.90 (m, 3H), 1.90-1.76 (m, 3H), 1.42 (s, 9H).

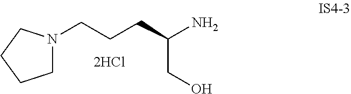 [SMILES: N[C@@H](CO)CCCN1CCCC1]

(R)-2-amino-5-(pyrrolidin-1-yl)pentan-1-ol hydrochloric acid salt (IS4-3)

Compound IS4-2 (2.98 g, 10.9 mmol, 1.0 equiv.) was dissolved in MeOH (20 mL) and HCl (4 M solution in dioxane, 8.15 mL, 32.6 mmol, 3.0 equiv.) was added at rt. The reaction mixture was stirred at rt for 2 h, at which point UPLC showed complete conversion. The reaction mixture was concentrated under reduced pressure, yielding 2.85 g (108% crude yield) of IS4-3. ¹H NMR (400 MHz, DMSO-d6) δ 10.68 (s, 1H), 7.99 (s, 2H), 5.33 (s, 1H), 3.63-3.54 (m, 1H), 3.54-3.41 (m, 3H), 3.15-3.02 (m, 3H), 3.02-2.86 (m, 2H), 2.04-1.93 (m, 2H), 1.93-1.81 (m, 2H), 1.81-1.68 (m, 2H), 1.67-1.47 (m, 2H).

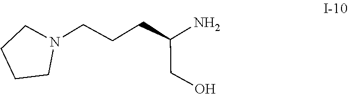 [SMILES: N[C@@H](CO)CCCN1CCCC1]

(R)-2-amino-5-(pyrrolidin-1-yl)pentan-1-ol (I-10)

Amberlyst A26(OH) (1 kg, >0.8 eq/L) was charged with MeOH (1 L) under mechanical stirrer and the mixture was allowed to stir for 30 min. The solvent was removed by filtration and the same sequence was repeated four times. Compound 3 (117.8 g, 483 mmol, 1.0 equiv.) was dissolved in MeOH (700 mL) and was added to the washed resin at rt. The reaction mixture was stirred at rt for 30 min. The solution was collected after filtration and the resin was washed with MeOH (700 mL) three times as before until UPLC shows no desired product was present in the solution. The organic solution was combined and concentrated under reduced pressure, yielding 81.1 g (97% crude yield) of I-10 free base as a yellow oil. ¹H NMR (400 MHz, CDCl₃-d) δ 3.57 (dd, 1H), 3.30 (dd, 1H), 2.84 (dtd, 1H), 2.58-2.41 (m, 6H), 1.78 (h, 5H), 1.70-1.41 (m, 3H), 1.40-1.26 (m, 1H).

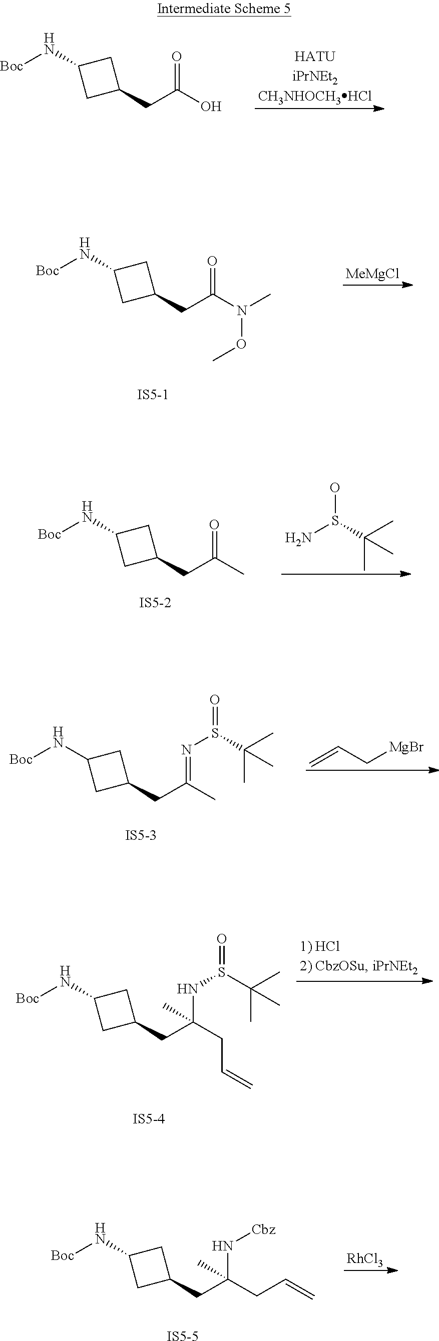

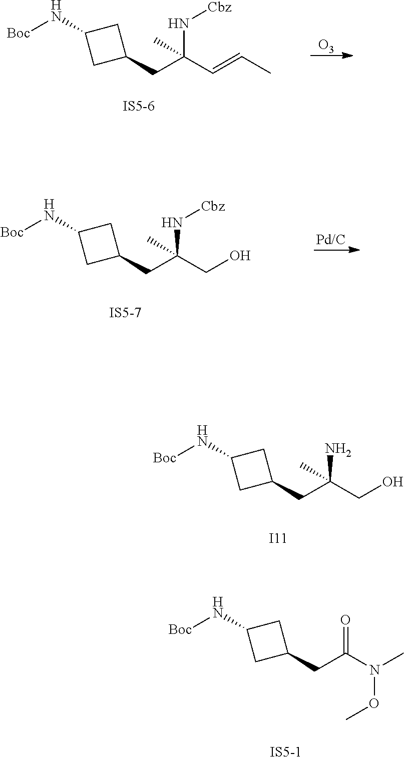

tert-butyl ((1r,3r)-3-(2-(methoxy(methyl)amino)-2-oxoethyl)cyclobutyl)carbamate (IS5-1)

To a solution of 2-((1r,3r)-3-((tert-butoxycarbonyl)amino)cyclobutyl)acetic acid (5 g, 21.8 mmol) in dichloromethane (109 mL) was added with methyoxyl(methyl)amine (3.17 g, 32.6 mmol), Hunig's base (11.3 mL, 65.3 mmol)) followed by HATU (12.3 g, 32.6 mmol). The reaction mixture was stirred at room temperature for 16 hrs, at which point UPLC showed complete conversion of the starting material to the desired mass. The reaction mixture was poured into 1 M NaOH and stirred vigorously for 10 mins, the organic layer was separated, and further washed with 2N HCl (2×), water (1×) and brine (1×). The washed solution was dried over sodium sulfate and concentrated. The crude is purified with ISCO to yield IS5-1 (5.9 g, 21.6 mmol, 99%). MS (ESI+) m/z: 295.2 [M+Na]+.

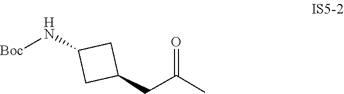 [SMILES: CC(=O)C[C@H]1C[C@H](NC(=O)OC(C)(C)C)C1]

tert-butyl ((1r,3r)-3-(2-oxopropyl)cyclobutyl)carbamate (IS5-2)

At −30° C., a solution of methyl magnesium chloride (1 M in THF, 17.1 mL, 51.4 mmol) was slowly added to a solution of Weinreb amide IS5-1 (5.9 g, 21.6 mmol) in THF (51.4 mL). The reaction mixture was stirred at −10 C for 30 mins. It was quenched with the NH₄C₁, and the crude mixture was extracted with EtOAc (3X). The combined organic layer was washed with brined, dried over MgSO₄, and concentrated. The crude mixture was purified by ISCO to yield IS5-2 (3.07 g, 13.5 mmol, 64%). MS (ESI+) m/z: 250.04 [M+Na]+; ¹H NMR (400 MHz, Chloroform-d) δ 4.71 (s, 1H), 4.19 (s, 1H), 2.64 (s, 2H), 2.59 (m, 1H), 2.11 (s, 3H), 2.06 (m, 4H), 1.43 (s, 9H).

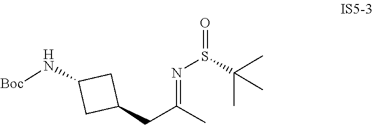 [SMILES: C/C(C[C@H]1C[C@H](NC(=O)OC(C)(C)C)C1)=N\[S@](=O)C(C)(C)C]

tert-butyl ((1R,3r)-3-((E)-2-(((R)-tert-butylsulfinyl)imino)propyl)cyclobutyl)carbamate (IS5-3)

To the ketone IS5-2 (2.9 g, 12.7 mmol) and (R)-2-methylpropane-2-sulfinamide (3.07 g, 25.4 mmol) in THF (25.4 mL) was added titanium ethoxide (5.32 mL, 25.4 mmol). The reaction mixture was heated to 70° C. for 24 h. THEED (8.97 g, 38 mmol) was added and the mixture was allowed to freely cool to 20° C. The reaction mixture was split between 1 N ammonium hydroxide (150 mL) and ethyl acetate (150 mL). Some solids were removed by filtration through a small pad of Celite®. The organic layer was dried over sodium sulfate and centrated. The crude was purified by ISCO to give IS5-3 (3 g, 9.07 mmol, 71%). MS (ESI+) m/z: 353.02 [M+Na]+.

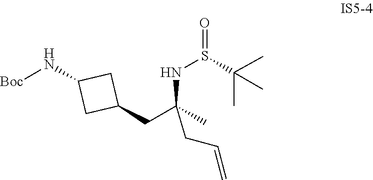 [SMILES: C=CC[C@@](C)(C[C@H]1C[C@H](NC(=O)OC(C)(C)C)C1)N[S@](=O)C(C)(C)C]

tert-butyl ((1R,3r)-3-((S)-2-(((R)-tert-butylsulfinyl)amino)-2-methylpent-4-en-1-yl)cyclobutyl)carbamate (IS5-4)

To imine IS5-3 (3 g, 9.07 mmol) in dichloromethane (18.1 mL) was added allyl magnesium bromide (1M in diethyl ether, 18.1 mL, 18.1 mmol) slowly at −20° C. at a rate such that precipitation of salts did not prevent stirring. The mixture was warmed to 0° C. for 1 h and then quenched with saturated aqueous ammonium chloride (50 mL). The organics were separated and concentrated. The residue was purified by ISCO to give IS5-4 (2.14 g, 5.74 mmol, 64%). MS (ESI+) m/z: 395.08 [M+Na]+. ¹H NMR (400 MHz, Chloroform-d) δ 5.80 (ddt, 1H), 5.18-5.05 (m, 2H), 4.74 (s, 1H), 3.17 (s, 1H), 2.52-2.39 (m, 1H), 2.35-2.23 (m, 2H), 2.06 (s, 2H), 1.73 (dd, 2H), 1.59 (s, 3H), 1.43 (s, 9H), 1.22 (s, 3H), 1.18 (s, 9H).

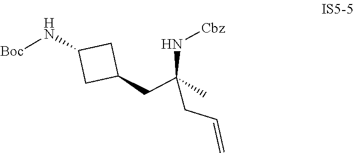 [SMILES: C=CC[C@@](C)(C[C@H]1C[C@H](NC(=O)OC(C)(C)C)C1)NC(=O)OCc1ccccc1]

tert-butyl ((1R,3r)-3-((S)-2-(((benzyloxy)carbonyl)amino)-2-methylpent-4-en-1-yl)cyclobutyl)carbamate (IS5-5)

A solution of the sulfinamide IS5-4 (2.14 g, 5.74 mmol) in THF/water (5:2 vol/vol, 14.5 mL) was added with conc. HCl (0.56 mL, 6.88 mmol), the reaction mixture was stirred at room temperature for 18 hours. Sat. aq. NaHCO₃ (10 mL) was added followed by N-(benzyloxycarbonyloxy)succinimide (1.5 g, 6.02 mmol). The reaction mixture was stirred at room temperature for 1 hour and extracted with EtOAc (2×). The combined organic extracts were washed with brine, dried over sodium sulfate, filtered and concentrated in vacuo. Crude was purified with ISCO to give IS5-5 (1.94 g, 84%, 4.81 mmol) as a white powder. MS (ESI+) m/z: 425.14 [M+Na]+; ¹H NMR (400 MHz, Chloroform-d) δ 7.42-7.29 (m, 5H), 5.75 (ddt, 1H), 5.10 (dd, 2H), 5.04 (s, 2H), 4.78-4.69 (m, 1H), 4.56 (s, 1H), 4.09 (s, 1H), 2.46 (m, 2H), 2.26 (dd, 1H), 2.07 (d, 4H), 1.78 (dd, 1H), 1.45 (s, 9H), 1.20 (s, 3H).

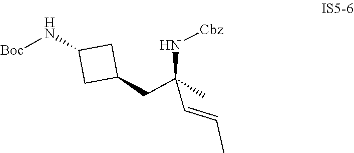 [SMILES: C/C=C/[C@@](C)(C[C@H]1C[C@H](NC(=O)OC(C)(C)C)C1)NC(=O)OCc1ccccc1]

tert-butyl ((1S,3r)-3-((R,E)-2-(((benzyloxy)carbonyl)amino)-2-methylpent-3-en-1-yl)cyclobutyl)carbamate (IS5-6)

To a solution of alkene IS5-5 (1.85 g, 4.59 mmol) in EtOH/water (9:1 vol/vol, 18.5 mL) was added with rhodium chloride hydrate (181 mg, 0.688 mmol). The reaction mixture is heated to 50° C. for 2.5 hr and 1H NMR confirmed complete conversion to the desired product. The reaction was concentrated and purified by ISCO to give IS5-6 (0.64 g, 1.58 mmol, 35%). MS (ESI+) m/z: 425.14 [M+Na]+; ¹H NMR (400 MHz, Chloroform-d) δ 7.44-7.29 (m, 5H), 5.49 (s, 1H), 5.05 (s, 2H), 4.72 (d, 2H), 4.07 (s, 1H), 2.36 (p, 1H), 2.09-1.87 (m, 4H), 1.69 (t, 2H), 1.60 (s, 3H), 1.45 (s, 9H), 1.34 (s, 3H).

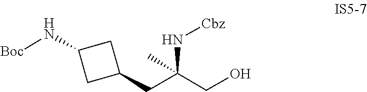 [SMILES: CC(C)(C)OC(=O)N[C@H]1C[C@H](C[C@](C)(CO)NC(=O)OCc2ccccc2)C1]

tert-butyl ((1S,3r)-3-((R)-2-(((benzyloxy)carbonyl)amino)-3-hydroxy-2-methylpropyl)cyclobutyl)carbamate (IS5-7)

To a stirred solution of alkene IS5-6 (0.64 g, 1.58 mmol) in methanol (32 mL) at −78° C. was passed ozone gas until the reaction color changed to blue. It was then purged with nitrogen for 5 mins until the color changed to colorless. Sodium borohydride (119 mg, 3.16 mmol) was added and the reaction stirred at −78° C. After 60 minutes, the reaction was deemed complete by TLC (aliquot quenched with NH4Cl and extracted with MTBE). The reaction was quenched with sat. aq NH₄Cl at ˜−70-50° C. MTBE was added and the reaction was warmed to room temperature. Organic layer was separated, washed with brine, dried over sodium sulfate and concentrated. Crude was purified with ISCO to provide IS5-7 (0.28 g, 45%). MS (ESI+) m/z: 415.12 [M+Na]+.

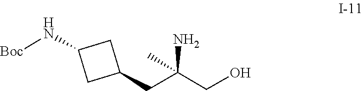 [SMILES: CC(C)(C)OC(=O)N[C@H]1C[C@H](C[C@@](C)(N)CO)C1]

tert-butyl ((1S,3r)-3-((R)-2-amino-3-hydroxy-2-methylpropyl)cyclobutyl)carbamate (I-11)

To A solution of Cbz-amino alcohol IS5-7 (278 mg, 0.71 mmol) in methanol (2 mL) and Pd/C (10% wt, 75.3 mg, 0.071 mmol), the reaction mixture was bubbled with hydrogen for 15 mins and stirred under an atmosphere of hydrogen for 1 hr. Upon completion, the mixture was filtered through Celite® with ethyl acetate and the filtrate was concentrated to give amino alcohol I-11 (180 mg, 0.7 mmol, 99%) as a clear oil. Crude material was used in the next step without further purification. MS (ESI+) m/z: 259.13 [M+H]+.

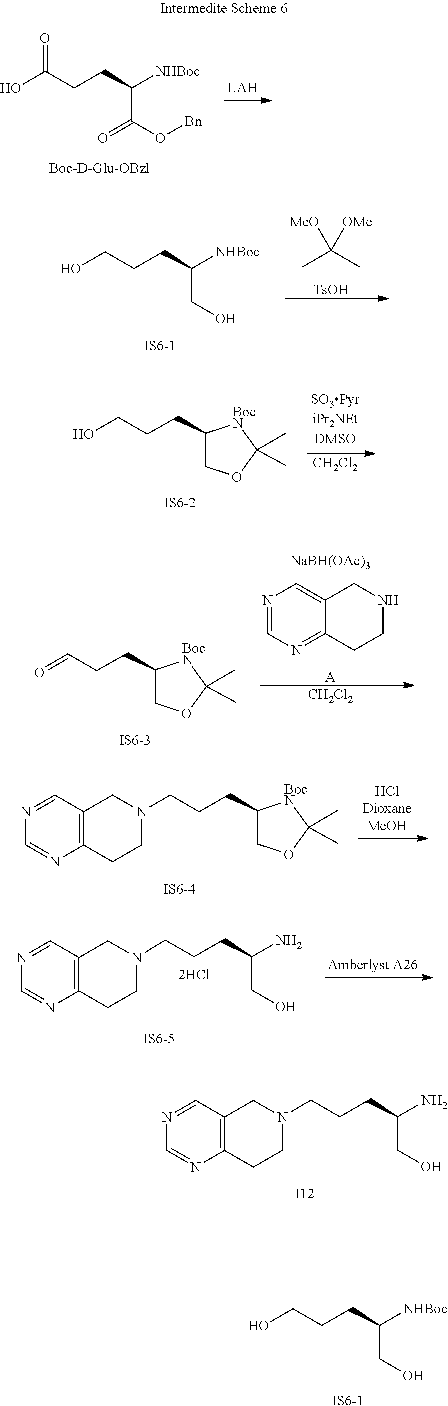

tert-butyl (R)-(1,5-dihydroxypentan-2-yl)carbamate (IS6-1)

A suspension of LiAlH₄ (1.0 M solution in THF, 800 mL, 800 mmol, 4.2 equiv.) was added drop-wise to a solution of Boc-D-Glu-OBzl (64.1 g, 190 mmol, 1.0 equiv.) in anhydrous tetrahydrofuran (474 mL) at 0° C. The internal temperature was kept under 10° C. Upon complete addition, the reaction mixture was allowed to stir at 0° C. for 0.5 h and was then warmed to rt for 1 h. The mixture was cooled to 0° C. and quenched by the cautious addition of water (30.4 mL), 3N NaOH solution (38.4 mL), and then water (84 mL). This mixture was dried with Na₂SO₄, and the precipitate was removed by filtration. The precipitate was washed with EtOAc, and the combined filtrate was concentrated under reduced pressure to give IS6-1. The material was used in the next step without further purification.

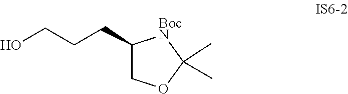

tert-butyl (R)-4-(3-hydroxypropyl)-2,2-dimethyloxazolidine-3-carboxylate (IS6-2)

A solution of compound IS6-1 (41.6 g, 189 mmol, 1.0 equiv.) in anhydrous methylene chloride (240 mL) was added 2,2-dimethoxypropane (231 mL, 1.89 mol, 10 equiv.) at 25° C. Then TsOH H₂O (3.59 g, 18.9 mmol, 0.1 equiv.) was added In an potion. The reaction mixture was allowed to stir at 25° C. for 4 h. The mixture was partitioned between EtOAc and sat. aq. NaHCO₃. The organic layer was washed with brine, dried over Na₂SO₄, and concentrated. The mixture was purified by silica gel chromatography (40% EtOAc in Heptane) to give 20.13 g (41% in two steps) compound IS6-2. ¹H NMR (400 MHz, Chloroform-d) δ 4.04-3.91 (m, 2H), 3.79-3.63 (m, 4H), 1.68-1.54 (m, 4H), 1.49 (s, 15H).

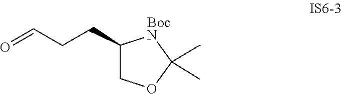

tert-butyl (R)-2,2-dimethyl-4-(3-oxopropyl)oxazolidine-3-carboxylate (IS6-3)

To a solution of compound IS6-2 (20.13 g, 77.5 mmol, 1.0 equiv) in DCM (155 mL) was added DMSO (44.0 mL, 620 mmol, 8.0 equiv) followed by hunig's base (53.9 mL, 310 mmol, 4.0 equiv) and cooled to 0° C. To this mixture was added SO₃·Pyr (24.6 g, 155 mmol, 2.0 equiv) in portions maintaining the internal temp below 5° C. The reaction mixture was allowed to stir at 0-5° C. for 1 h. To the batch was added MTBE and brine (500 mL+500 mL) and stirred for ˜10-15 mins. Organic layer was separated and washed with brine (4 times). Final organic layer was dried over sodium sulfate and concentrated to provide crude product IS6-3 and was used in next step without further purification.

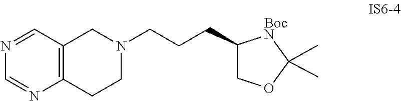

tert-butyl (R)-4-(3-(7,8-dihydropyrido[4,3-d]pyrimidin-6(5H)-yl)propyl)-2,2-dimethyloxazolidine-3-carboxylate (IS6-4)

To a solution of compound IS6-3 (19.9 g, 77.3 mmol, 1.0 equiv) in DCM (154 mL) was added Compound A (12.5 g, 92.7 mmol, 1.2 equiv) followed by AcOH (4.85 mL, 85.0 mmol, 1.1 equiv) and cooled to 0° C. To this mixture was added NaBH(OAc)₃ (24.3 g, 115 mmol, 1.5 equiv) in portions maintaining the internal temp below 5° C. The reaction mixture was allowed to stir at 25° C. for 16 h. Sat. aq. NaHCO₃ was added to the reaction mixture. The organic layer was separated and the aqueous layer was extracted with DCM (3 times). The combined organic layers were dried with sodium sulfate and concentrated in vacuo. The mixture was purified by silica gel chromatography (0-3-5% MeOH in DCM with 0.5% NH4OH) to gave 28.32 g (97.2% yield) of compound IS6-4.

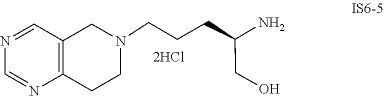 [SMILES: N[C@@H](CO)CCCN1CCc2ncncc2C1]

(R)-2-amino-5-(7,8-dihydropyrido[4,3-d]pyrimidin-6(5H)-yl)pentan-1-ol hydrochloric acid salt (IS6-5)

Compound IS6-4 (28.32 g, 75.1 mmol, 1.0 equiv.) was dissolved in MeOH (150 mL) and HCl (4 M solution in dioxane, 93.7 mL, 375 mmol, 5.0 equiv.) was added at rt. The reaction mixture was stirred at rt for 4 h, at which point UPLC showed complete conversion. The reaction mixture was concentrated under reduced pressure, yielding 22.9 g (100% crude yield) of Compound IS6-5.

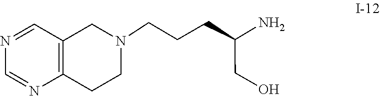 [SMILES: N[C@@H](CO)CCCN1CCc2ncncc2C1]

(R)-2-amino-5-(7,8-dihydropyrido[4,3-d]pyrimidin-6(5H)-yl)pentan-1-ol (I-12)

Amberlyst A26(OH) (200 g, >0.8 eq/L) was charged with MeOH (500 mL) under mechanical stirrer and the mixture was allowed to stir for 30 min. The solvent was removed by filtration and the same sequence was repeated four times. Compound 5 (22.9 g, 1.0 equiv.) was dissolved in MeOH (500 mL) and was added to the washed resin at rt. The reaction mixture was stirred at rt for 30 min. The solution was collected after filtration and the resin was washed with MeOH (500 mL) three times as before until UPLC shows no desired product was present in the solution. The organic solution was combined and concentrated under reduced pressure, yielding free base (16.97 g, 96.0% yield). ¹H NMR (400 MHz, CDCl₃) δ 8.99 (s, 1H), 8.41 (s, 1H), 3.66-3.57 (m, 3H), 3.51 (s, 2H), 3.32 (dd, 1H), 3.03 (t, 2H), 2.93-2.81 (m, 3H), 2.64-2.54 (m, 2H), 1.80-1.43 (m, 4H).

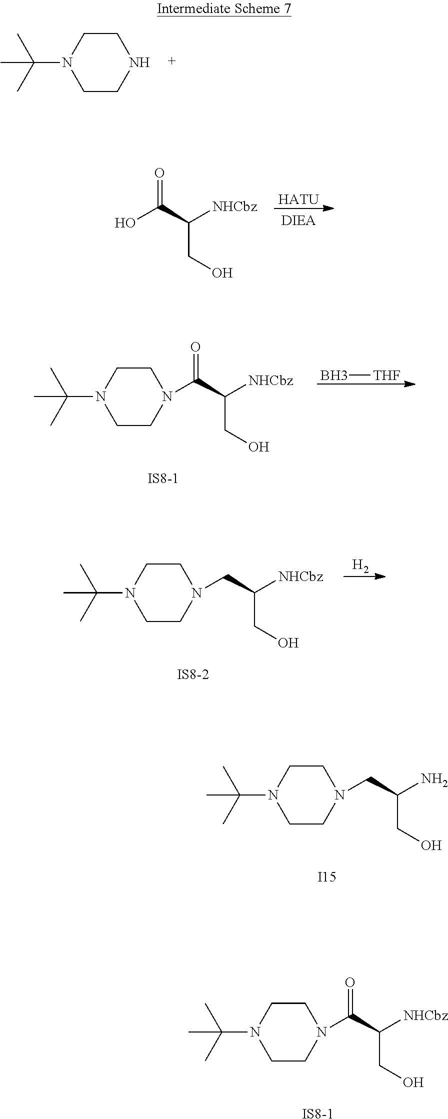

Benzyl (S)-(1-(4-(tert-butyl)piperazin-1-yl)-3-hydroxy-1-oxopropan-2-yl)carbamate (IS8-1)

To a solution of ((benzyloxy)carbonyl)-L-serine (3.1 g, 12.9 mmol, 1 eq) in DMF (16.5 mL) was added N,N-diisopropylethylamine (6.71 mL, 38.6 mmol, 3 eq) and 1-t-butyl-piperazine (2 g, 14.1 mmol, 1.1 eq). HATU (5.85 g, 15.4 mmol. 1.2 eq) was added in portions. The mixture was stirred at RT for 2 h. The mixture was diluted with DCM (40 mL) and was washed with water (4×80 mL) and brine (30 mL). The DCM solution was dried over Na₂SO₄ and was concentrated. The residue was purified by column chromatography on silica gel, eluting with a gradient of 100% DCM with 0.5% NH4OH to 20% MeOH in DCM with 0.5% NH4OH. This gave 3.9 g (83%) of the title compound as a white foam. MS (ESI+) m/z: 363.74 [M+H]+. ¹H NMR (400 MHz, Chloroform-d) δ 7.41-7.27 (m, 5H), 5.97 (d, J=8.3 Hz, 1H), 5.12 (q, J=12.2 Hz, 2H), 4.70 (dt, J=8.4, 4.2 Hz, 1H), 3.89-3.40 (m, 6H), 3.24 (s, 1H), 2.54 (qd, J=11.8, 11.2, 4.8 Hz, 4H), 1.06 (s, 9H).

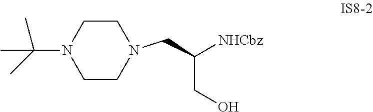 [SMILES: CC(C)(C)N1CCN(C[C@H](CO)NC(=O)OCc2ccccc2)CC1]

Benzyl (R)-(1-(4-(tert-butyl)piperazin-1-yl)-3-hydroxypropan-2-yl)carbamate (IS8-2)

A solution of benzyl (S)-(1-(4-(tert-butyl)piperazin-1-yl)-3-hydroxy-1-oxopropan-2-yl)carbamate (3.9 g, 10.7 mmol, 1 eq) in THF (22 mL) was cooled in an ice-water bath, and BH3-THF complex (33.3 mL, 1 M, 33.3 mmol, 3 eq) was added dropwise, maintaining an internal temperature below 10° C. The ice-water bath was removed, and the mixture was heated to reflux for 3 h. Upon cooling to rt, the mixture was quenched slowly (bubbling!) with MeOH (10 mL). After bubbling ceased, the solution was concentrated by rotovap. The residue was dissolved in MeOH (25 mL), and the solution was heated to reflux for 1.5 h. After cooling to rt and concentrating by rotovap, the residue was purified by column chromatography (40 g silica gel column, eluting with a gradient of 100% DCM with 0.5% NH₄OH to 20% MeOH in DCM with 0.5% NH4OH). This gave This gave 2 g (54%) of the title compound as a thick oil. MS (ESI+) m/z: 349.24 [M+H]+. ¹H NMR (400 MHz, Chloroform-d) δ 7.40-7.27 (m, 5H), 5.28 (d, J=13.2 Hz, 1H), 5.15-5.03 (m, 2H), 3.93-3.73 (m, 2H), 3.71-3.61 (m, 1H), 2.71-2.23 (m, 11H), 1.05 (s, 9H).

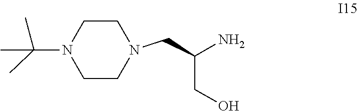

(R)-2-Amino-3-(4-(tert-butyl)piperazin-1-yl)propan-1-ol (115)

To a solution of benzyl (R)-(1-(4-(tert-butyl)piperazin-1-yl)-3-hydroxypropan-2-yl)carbamate (2 g,5.72 mmol) in MeOH (19 mL) was added 10% Pd/C (600 mg). After evacuating and back-filling with H₂, H₂ was bubbled through the mixture for 3 h. The mixture was filtered through celite, washing with MeOH, and was concentrated by rotovap. The residue was dried under vacuum to give 1.23 g (100%) of the title compound as a semi-solid. MS (ESI+) m/z: 201.18 [M+H]+. ¹H NMR (400 MHz, Chloroform-d) δ 3.69-3.54 (m, 2H), 3.43 (d, J=10.2 Hz, 4H), 3.19 (h, J=6.4, 5.9 Hz, 1H), 2.58 (ddd, J=31.2, 11.3, 5.9 Hz, 8H), 2.38 (dd, J=12.4, 7.3 Hz, 1H), 1.08 (s, 9H).

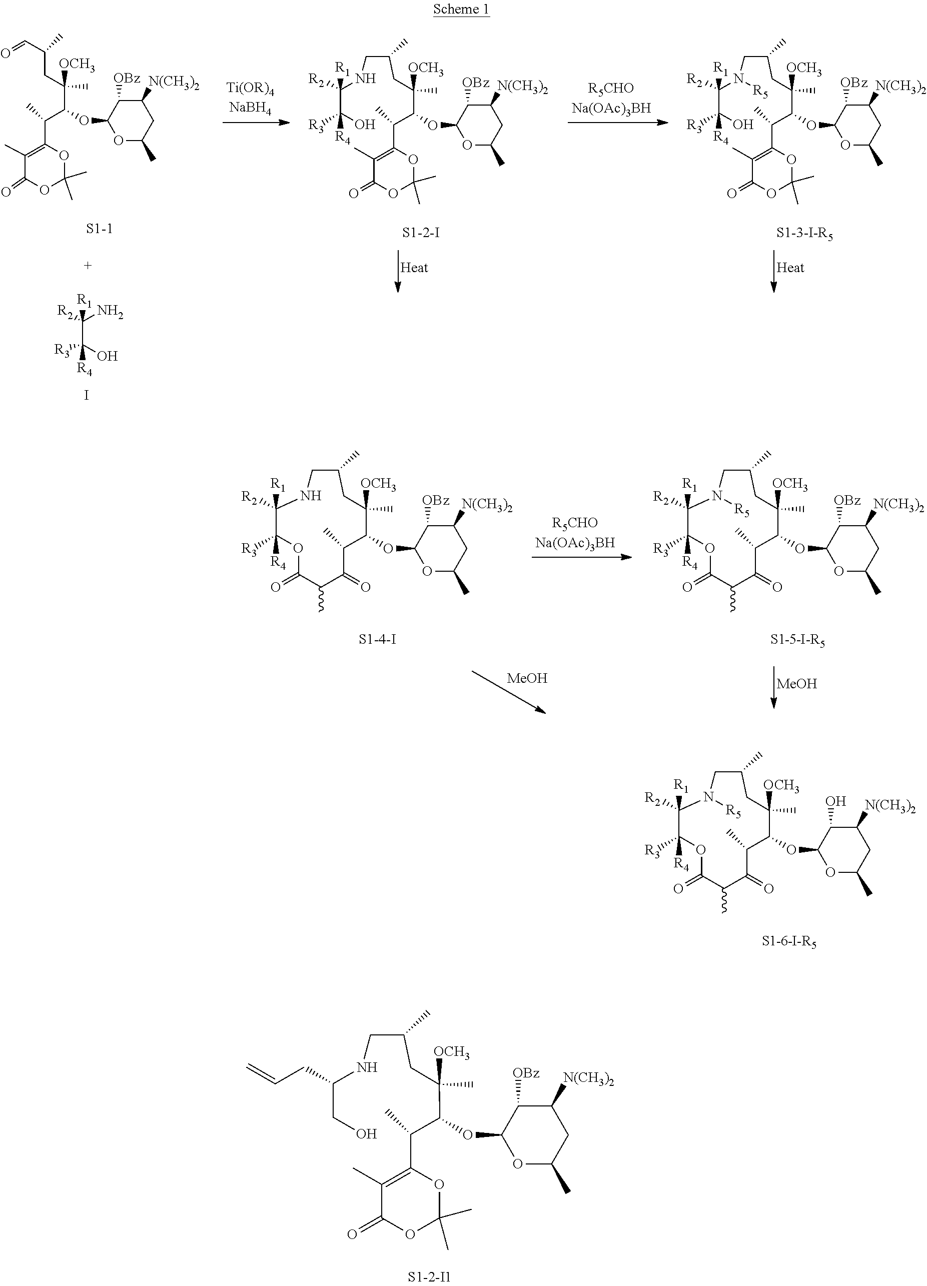

(2S,3R,4S,6R)-4-(Dimethylamino)-2-(((2R,3R,4R,6R)-7-(((S)-1-hydroxypent-4-en-2-yl)amino)-4-methoxy-4,6-dimethyl-2-(2,2,5-trimethyl-4-oxo-4H-1,3-dioxin-6-yl)heptan-3-yl)oxy)-6-methyltetrahydro-2H-pyran-3-yl benzoate (S1-2-I1)

(S)-2-aminopent-4-en-1-ol (343 mg, 3.40 mmol) and S1-1 (1.34 g, 2.27 mmol) were dissolved in EtOH (11.3 mL), and Ti(OEt)4 (0.946 mL, 4.54 mmol) was added. After 30 min, a small aliquot was removed from the reaction mixture and was added to a suspension of a small amount of NaBH4 in MeOH. LC/MS analysis showed approximately 90% conversion. Additional (S)-2-aminopent-4-en-1-ol (200 mg, 1.97 mmol) was added. After 30 min, a small aliquot was removed from the reaction mixture and was added to a suspension of a small amount of NaBH4 in MeOH. LC/MS analysis showed complete conversion. NaBH4 (171 mg, 4.54 mmol) was added. When gas evolution ceased, 30% aqueous NH4OH (6 mL) was added, and the mixture was filtered through a pad of Celite®, washing with EtOAc. The filtrate was washed with brine, was dried over Na2SO₄, was filtered, and was concentrated. The material was used without further purification. MS (ESI+) m/z: 675.25 [M+H]+.

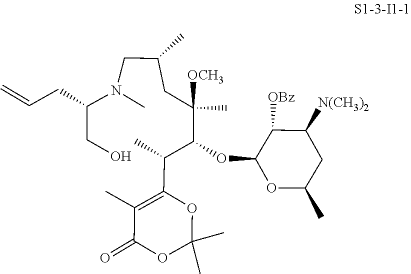

(2S,3R,4S,6R)-4-(Dimethylamino)-2-(((2R,3R,4R,6R)-7-(((S)-1-hydroxypent-4-en-2-yl)(methyl)amino)-4-methoxy-4,6-dimethyl-2-(2,2,5-trimethyl-4-oxo-4H-1,3-dioxin-6-yl)heptan-3-yl)oxy)-6-methyltetrahydro-2H-pyran-3-yl benzoate (S1-3-I1-1)

S1-2-I1 (1.53 g, 2.26 mmol) was dissolved in dichloromethane (10 mL) and Na(OAc)3BH (957 mg; 4.52 mmol) was added. Formaldehyde (37 wt % solution in water, 1.82 mL, 22.5 mmol) was added. After 15 min., additional Na(OAc)3BH (475 mg; 2.24 mmol) and formaldehyde (37 wt % solution in water, 0.30 mL, 3.7 mmol) were added. After 20 min., the reaction mixture was quenched by the addition of NaHCO₃(sat., aq. solution). The layers were separated, and the aqueous layer was extracted with dichloromethane (3 times). The combined dichloromethane extracts were dried over Na2SO4, were filtered, and were concentrated. The material was purified on 40 g of silica gel (elution with 2-10% MeOH-dichloromethane gradient containing 0.5% aqueous NH4OH) to give the title compound (1.20 g, 76%, 2 steps) as a thick oil. MS (ESI+) m/z: 689.26 [M+H]+. 1H NMR (400 MHz, Chloroform-d) δ 8.04 (dt, 2H), 7.61-7.51 (m, 1H), 7.44 (t, 2H), 5.82-5.66 (m, 1H), 5.16-4.95 (m, 3H), 4.70 (d, 1H), 3.87 (d, 1H), 3.55 (dq, 1H), 3.47 (dd, 1H), 3.33-3.18 (m, 2H), 3.06 (s, 3H), 2.88 (td, 1H), 2.81-2.66 (m, 1H), 2.49 (dd, 1H), 2.38-2.23 (m, 7H), 2.16 (s, 3H), 2.10 (dd, 1H), 1.92-1.75 (m, 5H), 1.73 (s, 3H), 1.68 (s, 3H), 1.64-1.55 (m, 1H), 1.55-1.42 (m, 1H), 1.38 (dd, 1H), 1.34-1.18 (m, 7H), 0.95 (d, 3H), 0.83 (d, 3H).

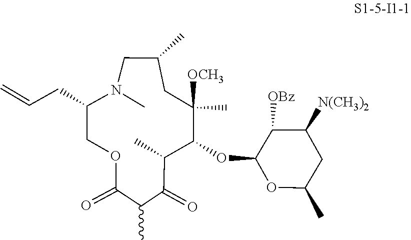

(2S,3R,4S,6R)-2-(((3S,6R,8R,9R,10R)-3-Allyl-8-methoxy-4,6,8,10,12-pentamethyl-11,13-dioxo-1-oxa-4-azacyclotridecan-9-yl)oxy)-4-(dimethylamino)-6-methyltetrahydro-2H-pyran-3-yl benzoate (S1-5-I1-1)

S1-3-I1-1 (1.19 g, 1.72 mmol) was concentrated three times from toluene. The material was dissolved in chlorobenzene (357 mL), and a stream of nitrogen was bubbled through the solution for 10 min. The mixture was heated at a bath temperature of 145° C. (approximately 130-135° C. internal temperature) overnight. The reaction was allowed to cool to rt and was concentrated. The residue was purified on 40 g of silica gel (elution with 2-10% MeOH-dichloromethane gradient containing 0.5% aqueous NH4OH) to give the title compound (835 mg, 77%). Mixture of C₂ epimers. MS (ESI+) m/z: 631.23 [M+H]+.

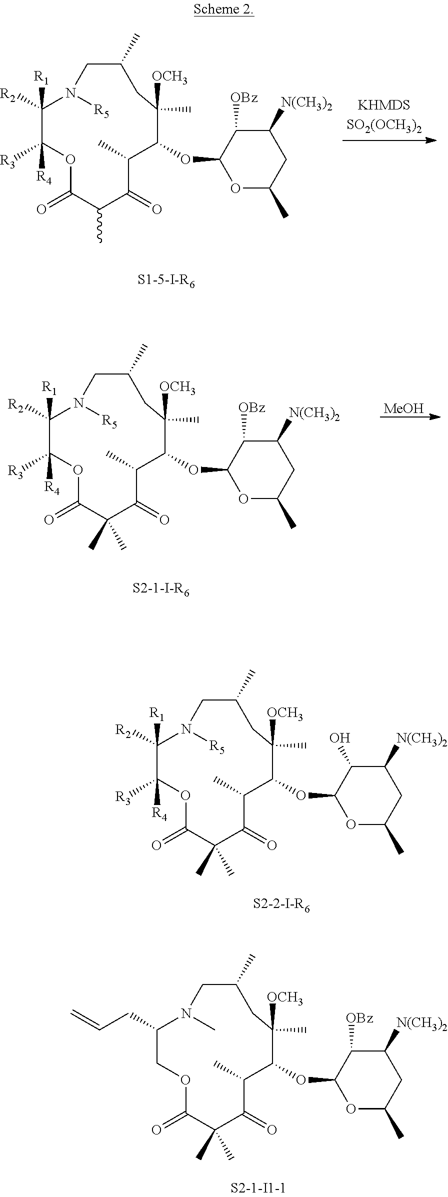

(2S,3R,4S,6R)-2-(((3S,6R,8R,9R,10R)-3-Allyl-8-methoxy-4,6,8,10,12,12-hexamethyl-11,13-dioxo-1-oxa-4-azacyclotridecan-9-yl)oxy)-4-(dimethylamino)-6-methyltetrahydro-2H-pyran-3-yl benzoate (S2-1-I1-1)

S1-5-I1-1 (834 mg, 1.32 mmol) was dissolved in 1,2-dimethoxyethane (6.6 mL), and the reaction mixture was cooled to −42° C. in a dry ice/acetonitrile bath. Potassium bis(trimethylsilyl)amide (1.0 M solution in THF; 1.71 mL, 1.71 mmol) was added. After 15 min, dimethyl sulfate (0.249 mL, 2.64 mmol) was added, and the bath was replaced with an ice/water batch. After 30 min., triethylamine (1.83 mL, 13.2 mmol) was added, and the reaction mixture was stirred at rt. After 20 min., the reaction was quenched by the addition of NH4C₁ (sat., aq. solution) and was extracted with dichloromethane (3 times). The combined extracts were over Na2SO₄, were filtered, and were concentrated. The residue was purified on 40 g of silica gel (elution with 2-10% MeOH-dichloromethane-0.5% NH4OH gradient) to give the title compound (551 mg, 64%). MS (ESI+) m/z: 645.24 [M+H]+. 1H NMR (400 MHz, Chloroform-d) δ 8.11-7.92 (m, 2H), 7.61-7.48 (m, 1H), 7.43 (t, 2H), 5.78 (dddd, 1H), 5.15-4.90 (m, 3H), 4.57 (d, 1H), 4.16 (dd, 1H), 4.01 (d, 1H), 3.96-3.80 (m, 1H), 3.58 (dtd, 1H), 3.41 (ddd, 1H), 3.08 (td, 1H), 2.99-2.86 (m, 1H), 2.81 (s, 3H), 2.36-2.22 (m, 7H), 2.19 (s, 3H), 2.01 (t, 2H), 1.75 (m, 7.1 Hz, 5H), 1.46-1.33 (m, 4H), 1.33-1.15 (m, 9H), 1.06-0.95 (d, 3H), 0.88 (d, 3H).

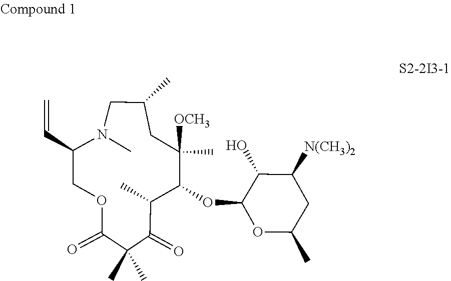

(3R,6R,8R,9R,10R)-9-(((2S,3R,4S,6R)-4-(Dimethylamino)-3-hydroxy-6-methyltetrahydro-2H-pyran-2-yl)oxy)-8-methoxy-4,6,8,10,12,12-hexamethyl-3-vinyl-1-oxa-4-azacyclotridecane-11,13-dione (S2-2-I3-1)

S2-1-I3-1 (18 mg, 0.029 mmol, prepared according to the methods of S2-1-I1-1) was dissolved in MeOH (2 mL), and the reaction mixture was heated to 65° C. (external temp.) for 3 h. The reaction mixture was cooled to rt and was concentrated under reduced pressure. The material was purified by HPLC (Atlantis T3 column, 5-30% MeCN-water-0.1% HCO2H) to give 6.35 mg of the title compound (6.35 mg) as a formate salt. MS (ESI+) m/z: 176.1 [M+3H]3+, 263.7 [M+2H]2+, 526.4 [M+H]+; 1H NMR (400 MHz, Methanol-d4) δ 8.54 (s, 2H), 5.97 (dt, 1H), 5.68 (s, 2H), 4.46 (d, 1H), 4.29-4.17 (m, 2H), 3.72 (dtt, 1H), 3.48-3.37 (m, 2H), 3.31 (tq, 2H), 3.06 (s, 3H), 2.95 (d, 1H), 2.82 (s, 1H), 2.75 (s, 6H), 2.00 (ddd, 1H), 1.53-1.25 (m, 16H), 1.05 (d, 3H).

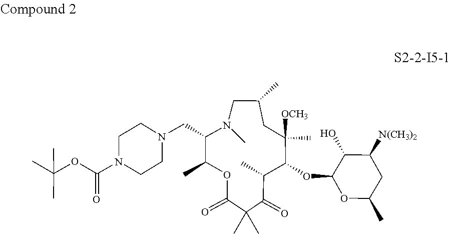 [SMILES: CO[C@]1(C)C[C@@H](C)CN(C)[C@@H](CN2CCN(C(=O)OC(C)(C)C)CC2)[C@H](C)OC(=O)C(C)(C)C(=O)[C@H](C)[C@H]1O[C@@H]1O[C@H](C)C[C@H](N(C)C)[C@H]1O]

tert-Butyl 4-(((2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-11,13-dioxo-1-oxa-4-azacyclotridecan-3-yl)methyl)piperazine-1-carboxylate (S2-2-I5-1)

Prepared according to the methods of S2-1-I1-1 and S2-2-I3-1 from I5 to provide the title compound as a formate salt. MS (ESI+) m/z: 713.6 [M+H]+; 1H NMR (400 MHz, Methanol-d) δ8.34 (s, 3H), 5.43 (dd, 1H), 4.49 (d, 1H), 4.10 (d, 1H), 3.87 (ddd, 1H), 3.73 (ddd, 1H), 3.55-3.37 (m, 7H), 3.17 (s, 3H), 3.11-3.03 (m, 1H), 3.02 (s, 3H), 2.96-2.85 (m, 2H), 2.82 (s, 6H), 2.62-2.52 (m, 3H), 2.52-2.40 (m, 2H), 2.26 (d, 1H), 2.08-1.97 (m, 1H), 1.82 (d, 1H), 1.57-1.48 (m, 4H), 1.44 (s, 9H), 1.38 (d, 4H), 1.37-1.33 (m, 9H), 1.31 (d, 3H), 1.06 (d, 3H).

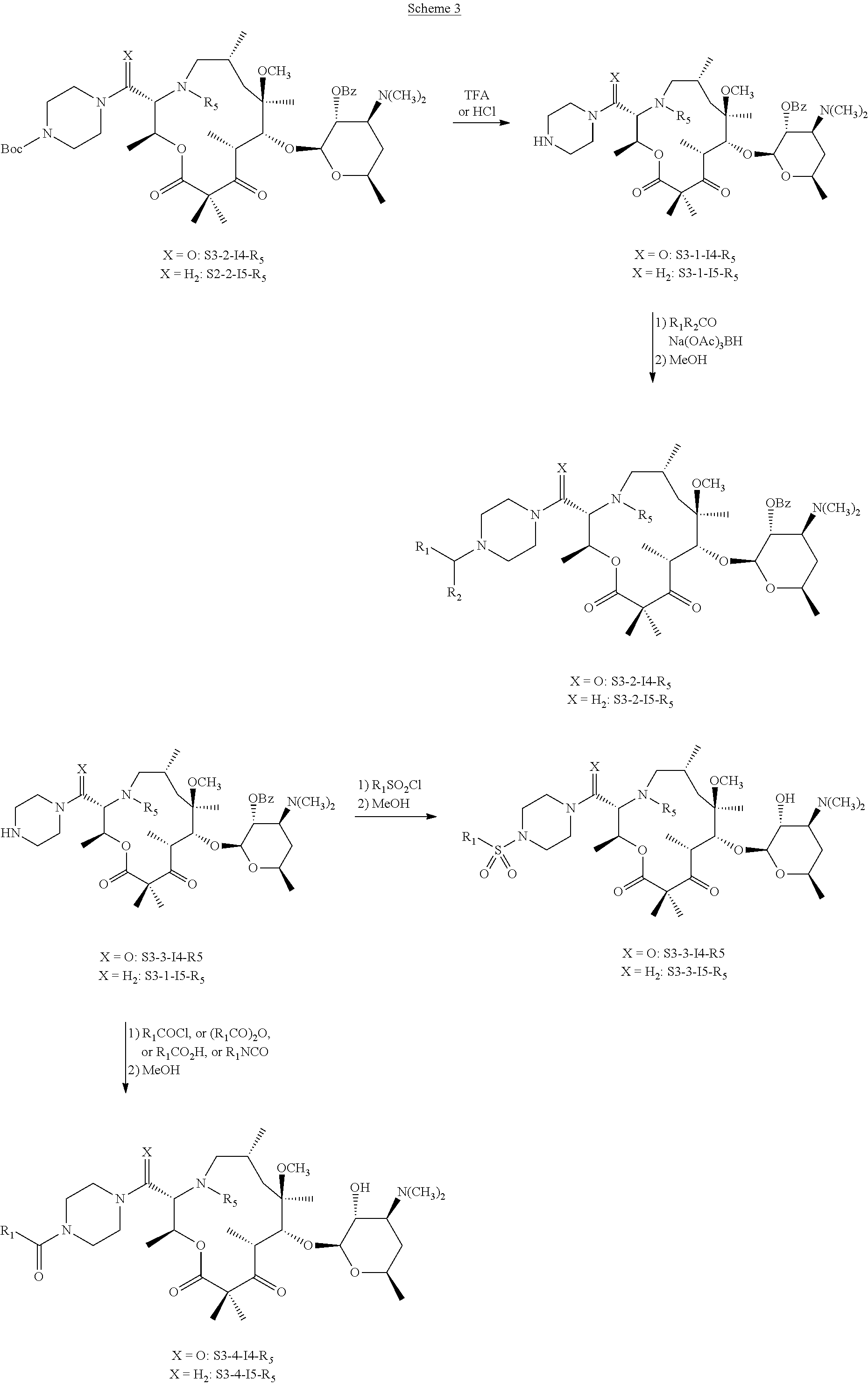

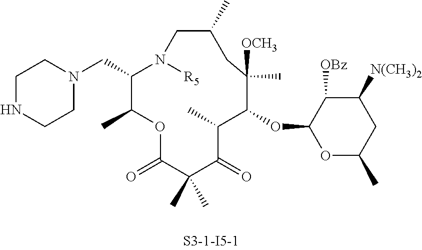

(2S,3R,4S,6R)-4-(dimethylamino)-2-(((2S,3S,6R,8R,9R,10R)-8-methoxy-2,4,6,8,10,12,12-heptamethyl-11,13-dioxo-3-(piperazin-1-ylmethyl)-1-oxa-4-azacyclotridecan-9-yl)oxy)-6-methyltetrahydro-2H-pyran-3-yl benzoate (S3-1-15-1)

S2-2-15-1 (430 mg, 0.526 mmol) was dissolved in dichloromethane (4.4 mL) and was cooled in an ice/water bath. Trifluoroacetic acid (0.5 mL, 6.52 mmol) was added, the ice/water bath was removed, and the reaction mixture was stirred at rt for 5.5 h. The reaction mixture was concentrated to a yellowish gum, was slowly treated with NaHCO₃(sat., aq., 10 mL), and was extracted with EtOAc (9 mL×4). The combined extracts were dried over Na2SO₄, were filtered, and were concentrated to give the crude title compound. MS (ESI+) m/z: 717.13 [M+H]+.

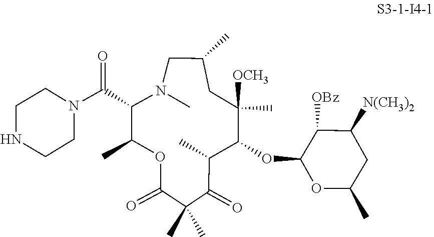

(2S,3R,4S,6R)-4-(dimethylamino)-2-(((2S,3R,6R,8R,9R,10R)-8-methoxy-2,4,6,8,10,12,12-heptamethyl-11,13-dioxo-3-(piperazine-1-carbonyl)-1-oxa-4-azacyclotridecan-9-yl)oxy)-6-methyltetrahydro-2H-pyran-3-yl benzoate (S3-1-14-1)

Prepared according to the methods of S3-1-15-1, substituting S2-2-14-1. This gave the title compound, which was used without further purification. MS (ESI+) m/z: 731.04 [M+H]+.

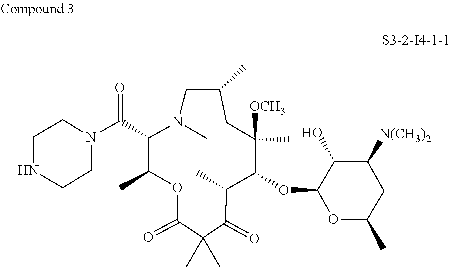 [SMILES: CO[C@]1(C)C[C@@H](C)CN(C)[C@@H](C(=O)N2CCNCC2)[C@H](C)OC(=O)C(C)(C)C(=O)[C@H](C)[C@H]1O[C@@H]1O[C@H](C)C[C@H](N(C)C)[C@H]1O]

(2S,3R,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-3-(piperazine-1-carbonyl)-1-oxa-4-azacyclotridecane-11,13-dione (S3-2-I4-1-1)

S3-1-I4-1 (35 mg, 0.048 mmol) was dissolved in MeOH (1 mL), and the reaction mixture was heated to 40° C. (external temp.) overnight. The reaction mixture was cooled to rt and was concentrated under reduced pressure. The material was purified by HPLC (Atlantis T3 column, 5-30% MeCN-water-0.1% HCO2H) to give 1.83 mg of the title compound as a formate salt. MS (ESI+) m/z: 627.42 [M+H]+; 1H NMR (400 MHz, Methanol-d) δ 8.29 (s, 4H), 5.27 (s, 1H), 4.46 (d, 1H), 4.30 (s, 1H), 4.14-3.87 (m, 4H), 3.81-3.63 (m, 2H), 3.62-3.50 (m, 2H), 3.46-3.35 (m, 2H), 3.31-3.12 (m, 3H), 2.92 (s, 3H), 2.74 (d, 10H), 2.35 (d, 1H), 2.08-1.84 (m, 2H), 1.62 (dd, 1H), 1.55-1.23 (m, 18H), 1.19 (d, 3H), 0.89 (d, 3H).

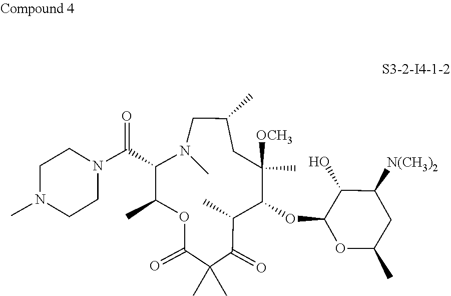 [SMILES: CO[C@]1(C)C[C@@H](C)CN(C)[C@@H](C(=O)N2CCN(C)CC2)[C@H](C)OC(=O)C(C)(C)C(=O)[C@H](C)[C@H]1O[C@@H]1O[C@H](C)C[C@H](N(C)C)[C@H]1O]

(2S,3R,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-3-(4-methylpiperazine-1-carbonyl)-1-oxa-4-azacyclotridecane-11,13-dione (S3-2-I4-1-2)

S3-1-14-1 (36.4 mg, 0.0497 mmol) was dissolved in dichloromethane (0.5 mL), and Na(OAc)3BH (20 mg, 0.094 mmol) followed by formaldehyde (37 wt % aqueous solution, 20.1 mg, 0.248 mmol) were added. After 14 h, the reaction mixture was quenched with NaHCO₃(sat., aq. solution) and was extracted with EtOAc (3 times). The combined extracts were dried over Na2SO4, were filtered, and were concentrated. The crude material was dissolved in methanol (1 mL), and the reaction mixture was heated to 40° C. external temperature overnight. The reaction was allowed to cool to rt and was concentrated. The residue was purified by HPLC (Atlantis T3 column, 5-50% MeCN-water-0.1% HCO2H) to give 9.45 mg of the title compound as a formate salt. MS (ESI+) m/z: 641.36 [M+H]+; 1H NMR (400 MHz, Methanol-d) δ8.33 (s, 3H), 5.30 (s, 1H), 4.48 (d, 1H), 4.31 (s, 1H), 4.03 (d, 1H), 3.94-3.80 (m, 2H), 3.80-3.68 (m, 2H), 3.67-3.54 (m, 2H), 3.49-3.36 (m, 2H), 2.95 (s, 3H), 2.82 (s, 6H), 2.76-2.65 (m, 6H), 2.65-2.52 (m, 2H), 2.44 (s, 3H), 2.42-2.31 (m, 1H), 2.06-1.98 (m, 1H), 1.92 (s, 1H), 1.71-1.62 (m, 1H), 1.52 (q, 1H), 1.47 (s, 3H), 1.42-1.35 (m, 4H), 1.35-1.29 (m, 9H), 1.20 (d, 3H), 0.92 (d, 3H).

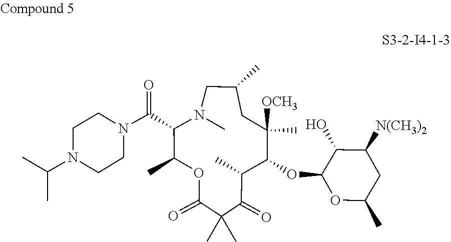 [SMILES: CO[C@]1(C)C[C@@H](C)CN(C)[C@@H](C(=O)N2CCN(C(C)C)CC2)[C@H](C)OC(=O)C(C)(C)C(=O)[C@H](C)[C@H]1O[C@@H]1O[C@H](C)C[C@H](N(C)C)[C@H]1O]

(2S,3R,6R,8R,9R,10R)-9-(((2S,3R,4S,6R)-4-(dimethylamino)-3-hydroxy-6-methyltetrahydro-2H-pyran-2-yl)oxy)-3-(4-isopropylpiperazine-1-carbonyl)-8-methoxy-2,4,6,8,10,12,12-heptamethyl-1-oxa-4-azacyclotridecane-11,13-dione (S3-2-I4-1-3)

Prepared according to the methods of S3-2-I4-1-2 from S3-1-I4-1 and acetone to provide the title compound as a formate salt. MS (ESI+) m/z: 669.44 [M+H]+; 1H NMR (400 MHz, Methanol-d) δ 8.50 (s, 2H), 5.08 (s, 1H), 4.48 (d, 1H), 4.09-3.89 (m, 2H), 3.87-3.54 (m, 6H), 3.50-3.35 (m, 3H), 2.91 (s, 3H), 2.80 (s, 6H), 2.79-2.72 (m, 1H), 2.71-2.44 (m, 8H), 2.15-1.93 (m, 2H), 1.75 (d, 2H), 1.52 (q, 1H), 1.42 (s, 3H), 1.39 (s, 3H), 1.31 (dd, 9H), 1.17 (d, 3H), 1.10 (d, 6H), 0.85 (d, 3H).

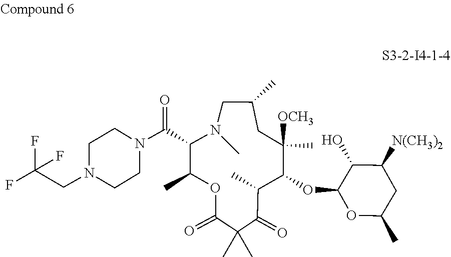 [SMILES: CO[C@]1(C)C[C@@H](C)CN(C)[C@@H](C(=O)N2CCN(CC(F)(F)F)CC2)[C@H](C)OC(=O)C(C)(C)C(=O)[C@H](C)[C@H]1O[C@@H]1O[C@H](C)C[C@H](N(C)C)[C@H]1O]

(2S,3R,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-3-(4-(2,2,2-trifluoroethyl)piperazine-1-carbonyl)-1-oxa-4-azacyclotridecane-11,13-dione (S3-2-I4-1-4)

Prepared according to the methods of S3-2-I4-1-1 from S3-1-I4-1 and 2,2,2-trifluoroacetaldehyde to provide the title compound as a formate salt. MS (ESI+) m/z: 709.29 [M+H]+; 1H NMR (400 MHz, Methanol-d) δ8.49 (s, 1H), 5.27 (s, 1H), 4.49 (d, 1H), 4.23 (s, 1H), 4.03 (d, 1H), 3.90-3.66 (m, 4H), 3.66-3.54 (m, 2H), 3.49-3.37 (m, 2H), 3.14 (q, 2H), 2.95 (s, 3H), 2.82 (s, 6H), 2.80-2.62 (m, 8H), 2.33 (s, 1H), 2.09-1.98 (m, 1H), 1.95-1.81 (m, 1H), 1.70 (d, 1H), 1.54 (q, 1H), 1.50-1.41 (m, 4H), 1.41-1.23 (m, 13H), 1.20 (d, 3H), 0.91 (d, 3H).

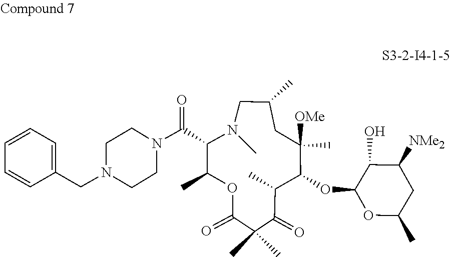

(2S,3R,6R,8R,9R,10R)-3-(4-benzylpiperazine-1-carbonyl)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-1-oxa-4-azacyclotridecane-11,13-dione (S3-2-I4-1-5)

Prepared according to the methods of S3-2-I4-1-1 from S3-1-I4-1 and benzaldehyde to provide the title compound as a formate salt. MS (ESI+) m/z: 717.5 [M+H]+; ¹H NMR (400 MHz, METHANOL-d4) δ=7.42-7.22 (m, 5H), 5.13-5.01 (m, 1H), 4.63-4.51 (m, 1H), 4.38 (d, 1H), 4.00 (d, 1H), 3.90 (d, 1H), 3.76-3.50 (m, 9H), 3.29-3.21 (m, 1H), 2.89 (s, 3H), 2.80-2.64 (m, 2H), 2.55-2.44 (m, 7H), 2.39 (s, 6H), 2.35-2.25 (m, 1H), 2.16 (s, 1H), 2.06 (br d, 1H), 1.85-1.6 in, 3H), 1.39 (d, 6H), 1.33-1.22 (m, 10H), 1.18-1.05 (m, 4H), 0.84 (d, 3H).

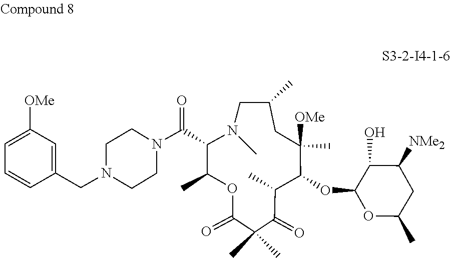

(2S,3R,6R,8R,9R,10R)-9-(((2S,3R,4S,6R)-4-(dimethylamino)-3-hydroxy-6-methyltetrahydro-2H-pyran-2-yl)oxy)-8-methoxy-3-(4-(3-methoxybenzyl)piperazine-1-carbonyl)-2,4,6,8,10,12,12-heptamethyl-1-oxa-4-azacyclotridecane-11,13-dione (S3-2-I4-1-6)

Prepared according to the methods of S3-2-I4-1-1 from S3-1-14-1 and 3-methoxybenzaldehyde to provide the title compound as a formate salt. MS (ESI+) m/z: 747.5 [M+H]+; ¹H NMR (400 MHz, METHANOL-d4) δ=8.55 (br s, 0.2H), 7.23 (t, 1H), 6.97-6.88 (m, 2H), 6.84 (br d, 1H), 5.12-5.00 (m, 1H), 4.39 (d, 1H), 4.04-3.96 (m, 1H), 3.89 (br d, 1H), 3.82-3.47 (m, 12H), 2.94-2.73 (m, 4H), 2.55-2.37 (m, 14H), 2.30 (br t, 1H), 2.04 (br d, 1H), 1.86-1.64 (m, 3H), 1.39 (d, 6H), 1.32-1.21 (m, 10H), 1.19-1.04 (m, 4H), 0.84 (br d, 3H).

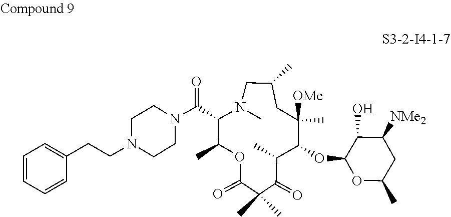

(2S,3R,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-3-(4-phenethylpiperazine-1-carbonyl)-1-oxa-4-azacyclotridecane-11,13-dione (S3-2-I4-1-7)

Prepared according to the methods of S3-2-I4-1-1 from S3-1-I4-1 and phenylacetaldehyde to provide the title compound as a formate salt. MS (ESI+) m/z: 731.5 [M+H]+; ¹H NMR (400 MHz, METHANOL-d4) δ=7.30-7.14 (m, 5H), 5.15-5.02 (m, 1H), 4.38 (d, 1H), 4.00 (d, 1H), 3.91 (d, 1H), 3.81-3.48 (m, 7H), 3.29-3.23 (m, 1H), 2.93-2.80 (m, 5H), 2.76-2.65 (m, 1H), 2.67-2.46 (m, 10H), 2.40 (s, 6H), 2.31 (s, 1H), 2.09-2.00 (m, 1H), 1.85-1.60 (m, 3H), 1.43-1.21 (m, 17H), 1.19-1.05 (m, 4H), 0.85 (d, 3H).

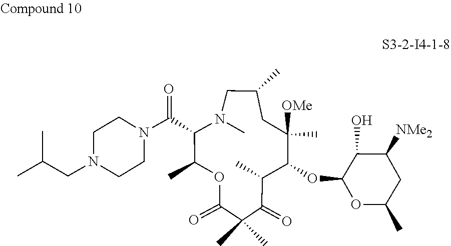

(2S,3R,6R,8R,9R,10R)-9-(((2S,3R,4S,6R)-4-(dimethylamino)-3-hydroxy-6-methyltetrahydro-2H-pyran-2-yl)oxy)-3-(4-isobutylpiperazine-1-carbonyl)-8-methoxy-2,4,6,8,10,12,12-heptamethyl-1-oxa-4-azacyclotridecane-11,13-dione (S3-2-I4-1-8)

Prepared according to the methods of S3-2-I4-1-1 from S3-1-14-1 and isobutyraldehyde to provide the title compound as a formate salt. MS (ESI+) m/z: 683.5 [M+H]+; ¹H NMR (400 MHz, METHANOL-d4) δ=5.07 (br dd, 1H), 4.40 (d, 1H), 4.04-3.84 (m, 2H), 3.75-3.49 (m, 6H), 2.90 (s, 4H), 2.57-2.44 (m, 11H), 2.43-2.24 (m, 4H), 2.18-1.99 (m, 3H), 1.90-1.63 (m, 4H), 1.39 (br d, 7H), 1.34-1.22 (m, 10H), 1.20-1.06 (m, 4H), 0.93 (d, 6H), 0.85 (br d, 3H).

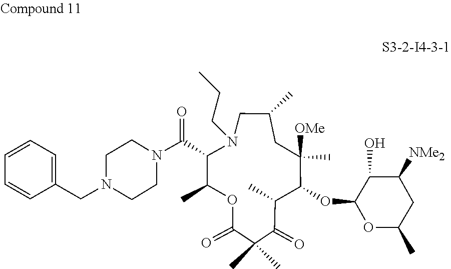

(2S,3R,6R,8R,9R,10R)-3-(4-benzylpiperazine-1-carbonyl)-9-(((2S,3R,4S,6R)-4-(dimethylamino)-3-hydroxy-6-methyltetrahydro-2H-pyran-2-yl)oxy)-8-methoxy-2,6,8,10,12,12-hexamethyl-4-propyl-1-oxa-4-azacyclotridecane-11,13-dione (S3-2-I4-3-1)

Prepared according to the methods of S3-2-I4-1-1 from S3-1-14-3 and benzaldehyde to provide the title compound as a formate salt. MS (ESI+) m/z: 745.5 [M+H]+; ¹H NMR (400 MHz, METHANOL-d4) δ=7.41-7.18 (m, 5H), 5.10 (br s, 1H), 4.35 (d, 1H), 4.03 (br d, 2H), 3.77-3.49 (m, 8H), 3.29-3.18 (m, 2H), 2.86 (s, 3H), 2.73-2.62 (m, 2H), 2.56-2.33 (m, 12H), 1.98 (br d, 1H), 1.85-1.73 (m, 1H), 1.64 (br d, 1H), 1.54-1.44 (m, 2H), 1.38 (d, 5H), 1.33-1.21 (m, 10H), 1.19-1.05 (m, 4H), 0.95-0.81 (m, 6H).

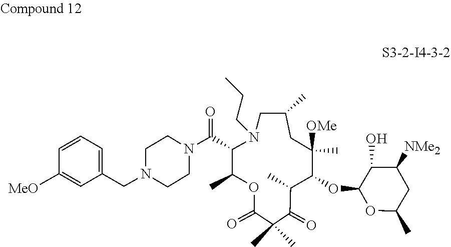

(2S,3R,6R,8R,9R,10R)-9-(((2S,3R,4S,6R)-4-(dimethylamino)-3-hydroxy-6-methyltetrahydro-2H-pyran-2-yl)oxy)-8-methoxy-3-(4-(3-methoxybenzyl)piperazine-1-carbonyl)-2,6,8,10,12,12-hexamethyl-4-propyl-1-oxa-4-azacyclotridecane-11,13-dione (S3-2-I4-3-2)

Prepared according to the methods of S3-2-I4-1-1 from S3-1-I4-3 and 3-methoxybenzaldehyde to provide the title compound as a formate salt. MS (ESI+) m/z: 775.5 [M+H]+; ¹H NMR (400 MHz, METHANOL-d4) δ=7.24 (t, 1H), 6.95-6.88 (m, 2H), 6.84 (dd, 1H), 5.09 (br s, 1H), 4.36 (d, 1H), 4.10-3.96 (m, 2H), 3.79 (s, 3H), 3.74-3.46 (m, 8H), 2.85 (s, 3H), 2.77-2.61 (m, 2H), 2.56-2.30 (m, 12H), 1.97 (br d, 1H), 1.78 (br dd, 2H), 1.63 (br s, 1H), 1.54-1.06 (m, 23H), 0.95-0.78 (m, 7H).

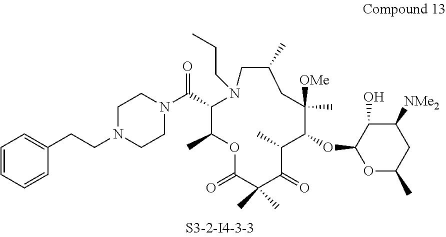

(2S,3R,6R,8R,9R,10R)-9-(((2S,3R,4S,6R)-4-(dimethylamino)-3-hydroxy-6-methyltetrahydro-2H-pyran-2-yl)oxy)-8-methoxy-2,6,8,10,12,12-hexamethyl-3-(4-phenethylpiperazine-1-carbonyl)-4-propyl-1-oxa-4-azacyclotridecane-11,13-dione (S3-2-I4-3-3)

Prepared according to the methods of S3-2-I4-1-1 from S3-1-I4-3 and phenylacetaldehyde to provide the title compound as a formate salt. MS (ESI+) m/z: 759.5 [M+H]+; ¹H NMR (400 MHz, METHANOL-d4) δ=7.29-7.15 (m, 5H), 5.11 (br s, 1H), 4.38-4.32 (m, 1H), 4.08-3.99 (m, 2H), 3.75-3.48 (m, 7H), 3.29-3.22 (m, 1H), 2.92-2.78 (m, 5H), 2.72-2.51 (m, 8H), 2.45-2.31 (m, 8H), 2.18-2.14 (m, 1H), 1.99 (br d, 1H), 1.84-1.71 (m, 2H), 1.71-1.59 (m, 1H), 1.57-1.43 (m, 3H), 1.39 (d, 6H), 1.33-1.09 (m, 16H), 0.95-0.83 (m, 7H).

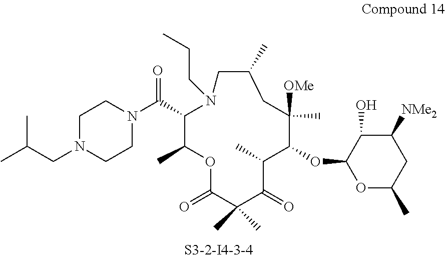

(2S,3R,6R,8R,9R,10R)-9-(((2S,3R,4S,6R)-4-(dimethylamino)-3-hydroxy-6-methyltetrahydro-2H-pyran-2-yl)oxy)-3-(4-isobutylpiperazine-1-carbonyl)-8-methoxy-2,6,8,10,12,12-hexamethyl-4-propyl-1-oxa-4-azacyclotridecane-11,13-dione (S3-2-I4-3-4)

Prepared according to the methods of S3-2-I4-1-1 from S3-1-14-3 and isobutyraldehyde to provide the title compound as a formate salt. MS (ESI+) m/z: 711.5 [M+H]+; ¹H NMR (400 MHz, METHANOL-d₄) δ=5.18-5.03 (m, 1H), 4.35 (d, 1H), 4.07-3.98 (m, 2H), 3.74-3.52 (m, 6H), 3.29-3.21 (m, 1H), 2.87 (s, 3H), 2.76-2.61 (m, 2H), 2.51-2.29 (m, 12H), 2.13 (d, 2H), 1.99 (br d, 1H), 1.91-1.73 (m, 3H), 1.71-1.59 (m, 1H), 1.56-1.43 (m, 3H), 1.39 (d, 6H), 1.33-1.21 (m, 10H), 1.15 (d, 3H), 0.93 (d, 7H), 0.90-0.82 (m, 5H).

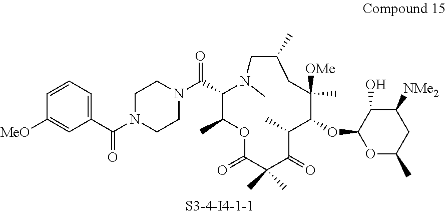

(2S,3R,6R,8R,9R,10R)-9-(((2S,3R,4S,6R)-4-(dimethylamino)-3-hydroxy-6-methyltetrahydro-2H-pyran-2-yl)oxy)-8-methoxy-3-(4-(3-methoxybenzoyl)piperazine-1-carbonyl)-2,4,6,8,10,12,12-heptamethyl-1-oxa-4-azacyclotridecane-11,13-dione (S3-4-I4-1-1)

S3-2-I4-1 (61 mg, 72.19 umol) in CH₂Cl₂ (2 mL) was added DMAP (3 mg, 21.66 umol), DIPEA (47 mg, 360.97 umol, 62.87 uL) and 3-methoxybenzoyl chloride (37 mg, 216.58 umol, 29.56 uL) at 20° C., the reaction mixture was stirred at same temperature for 2 h. The reaction was quenched with sat. aq. NaHCO₃ (2 mL), and extracted with CH₂Cl₂ (2 mL×3). The combined organic layers were dried (Na₂SO₄), filtered, and concentrated in vacuum to give the crude amide (75 mg, crude) as a yellow oil. It was dissolved in MeOH (8 mL) and heated to 55° C. for 16 h. The reaction mixture was concentrated. Crude residue was purified by Prep.-HPLC to give the title compound as a formate salt. MS (ESI+) m/z: 761.5 [M+H]+; ¹H NMR (400 MHz, METHANOL-d4) δ=8.41 (br d, 1H), 7.40 (t, 1H), 7.09-6.99 (m, 3H), 5.19-5.05 (m, 1H), 4.48 (d, 1H), 4.00 (br d, 2H), 3.94-3.53 (m, 13H), 3.49-3.34 (m, 4H), 2.95-2.87 (m, 3H), 2.86-2.78 (m, 6H), 2.66-2.33 (m, 4H), 2.02 (br d, 2H), 1.83-1.67 (m, 2H), 1.53 (br d, 1H), 1.44-1.12 (m, 20H), 0.86 (br d, 3H).

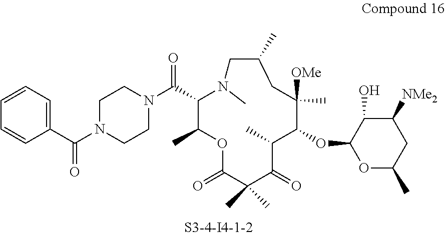

(2S,3R,6R,8R,9R,10R)-3-(4-benzoylpiperazine-1-carbonyl)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-1-oxa-4-azacyclotridecane-11,13-dione (S3-4-I4-1-2)

Prepared according to the methods of S3-4-I4-1-1 from S3-1-I4-1 and benzoyl chloride to provide the title compound as a formate salt. MS (ESI+) m/z: 731.5 [M+H]+; ¹H NMR (400 MHz, METHANOL-d4) δ=7.53-7.44 (m, 5H), 5.13-5.00 (m, 2H), 4.39 (br d, 1H), 4.09-3.38 (m, 13H), 2.96-2.75 (m, 4H), 2.53-2.42 (m, 9H), 2.34 (br t, 1H), 2.04 (br d, 1H), 1.86-1.66 (m, 3H), 1.42-1.23 (m, 16H), 1.20-1.08 (m, 4H), 0.85 (br d, 3H).

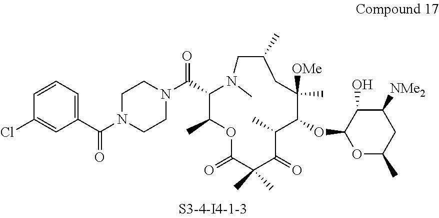

(2S,3R,6R,8R,9R,10R)-3-(4-(3-chlorobenzoyl)piperazine-1-carbonyl)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-1-oxa-4-azacyclotridecane-11,13-dione (S3-4-I4-1-3)

Prepared according to the methods of S3-4-I4-1-1 from S3-1-I4-1 and 3-chlorobenzoyl chloride to provide the title compound as a formate salt. MS (ESI+) m/z: 765.5 [M+H]+; ¹H NMR (400 MHz, METHANOL-d4) δ=8.44 (br s, 1H), 7.55-7.44 (m, 3H), 7.43-7.36 (m, 1H), 5.11 (br s, 1H), 4.48 (d, 1H), 4.00 (br d, 1H), 3.90-3.51 (m, 9H), 3.48-3.36 (m, 3H), 2.90 (br s, 3H), 2.81 (s, 6H), 2.55 (br s, 3H), 2.43 (br s, 1H), 2.20-2.14 (m, 1H), 2.02 (br d, 1H), 1.90-1.72 (s, 2H), 1.52 (q, 1H), 1.42 (s, 3H), 1.38 (br s, 3H), 1.34-1.22 (m, 10H), 1.19 (br d, 4H), 0.86 (br d, 3H).

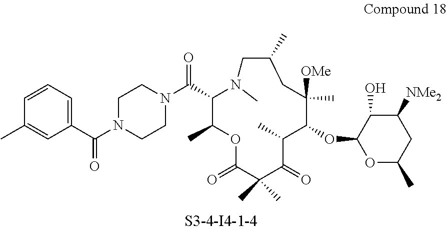

(2S,3R,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-3-(4-(3-methylbenzoyl)piperazine-1-carbonyl)-1-oxa-4-azacyclotridecane-11,13-dione (S3-4-I4-1-4)

Prepared according to the methods of S3-4-I4-1-1 from S3-1-I4-1 and 3-methylbenzoyl chloride to provide the title compound as a formate salt. MS (ESI+) m/z: 745.5 [M+H]+; ¹H NMR (400 MHz, METHANOL-d4) δ=8.56 (br s, 1H), 7.40-7.30 (m, 2H), 7.27 (s, 1H), 7.24 (br d, 1H), 5.07-5.20 (m, 1H), 4.47 (d, 1H), 4.05-3.34 (m, 15H), 2.89 (br s, 3H), 2.79 (s, 6H), 2.52 (br s, 3H), 2.39 (s, 4H), 2.15-2.01 (m, 2H), 1.73 (br d, 2H), 1.56-1.46 (m, 1H), 1.44-1.34 (m, 6H), 1.34-1.21 (m, 10H), 1.18 (br d, 5H), 0.84 (br d, 3H).

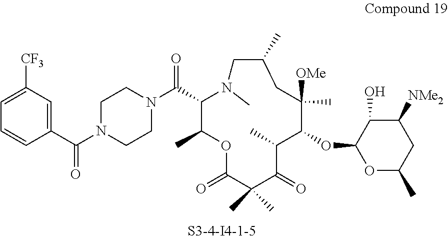

(2S,3R,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-3-(4-(3-(trifluoromethyl)benzoyl)piperazine-1-carbonyl)-1-oxa-4-azacyclotridecane-11,13-dione (S3-4-I4-1-5)

Prepared according to the methods of S3-4-I4-1-1 from S3-1-I4-1 and 3-trifluoromethylbenzoyl chloride to provide the title compound as a formate salt. MS (ESI+) m/z: 799.5 [M+H]+; ¹H NMR (400 MHz, METHANOL-d4) δ=8.54 (br s, 1H), 7.84-7.77 (m, 2H), 7.76-7.67 (m, 2H), 5.12-5.01 (m, 2H), 4.76-4.53 (m, 6H), 4.44 (br d, 1H), 4.04-3.43 (m, 12H), 3.43-3.33 (m, 1H), 3.21-3.10 (m, 1H), 2.88 (br s, 3H), 2.66 (s, 6H), 2.50 (br s, 3H), 2.45-2.29 (m, 1H), 2.04 (br d, 1H), 1.93 (br d, 1H), 1.82-1.63 (m, 2H), 1.48-1.34 (m, 7H), 1.33-1.23 (m, 9H), 1.22-1.07 (m, 5H), 0.90-0.80 (m, 3H).

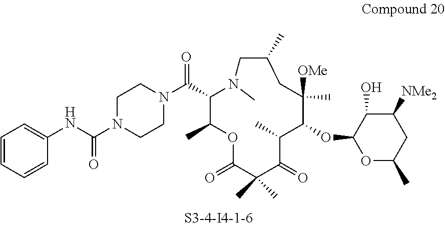

4-((2S,3R,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-11,13-dioxo-1-oxa-4-azacyclotridecane-3-carbonyl)-N-phenylpiperazine-1-carboxamide (S3-4-I4-1-6)

Prepared according to the methods of S3-4-I4-1-1 from S3-1-14-1 and phenyl isocyanate to provide the title compound as a formate salt. MS (ESI+) m/z: 746.5 [M+H]+; ¹H NMR (400 MHz, METHANOL-d4) δ=7.36 (br d, 2H), 7.27 (t, 2H), 7.03 (t, 1H), 5.16-5.04 (m, 1H), 4.62-4.52 (m, 1H), 4.40 (d, 1H), 4.04-3.90 (m, 2H), 3.82-3.54 (m, 10H), 2.95-2.83 (m, 4H), 2.50 (br d, 8H), 2.45-2.24 (m, 2H), 2.07 (br d, 1H), 1.91-1.66 (m, 3H), 1.56-1.23 (m, 17H), 1.23-1.08 (m, 5H), 0.86 (br d, 3H).

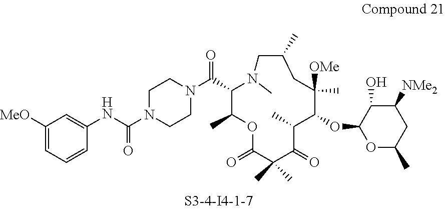

4-((2S,3R,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-11,13-dioxo-1-oxa-4-azacyclotridecane-3-carbonyl)-N-(3-methoxyphenyl)piperazine-1-carboxamide (S3-4-I4-1-7)

Prepared according to the methods of S3-4-I4-1-1 from S3-1-14-1 and 3-methoxyphenyl isocyanate to provide the title compound as a formate salt. MS (ESI+) m/z: 776.5 [M+H]+; ¹H NMR (400 MHz, METHANOL-d4) δ=8.54 (br s, 1H), 7.15 (t, 1H), 7.04 (t, 1H), 6.92 (dd, 1H), 6.60 (dd, 1H), 5.08 (br dd, 1H), 4.41 (d, 1H), 4.06-3.89 (m, 2H), 3.86-3.47 (m, 14H), 3.02-2.84 (m, 4H), 2.66-2.47 (m, 9H), 2.40-2.25 (m, 1H), 2.06 (br d, 1H), 1.92-1.65 (m, 3H), 1.51-1.06 (m, 22H), 0.85 (d, 3H).

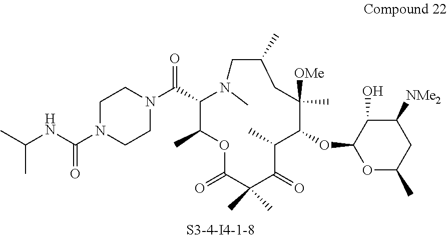

4-((2S,3R,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-11,13-dioxo-1-oxa-4-azacyclotridecane-3-carbonyl)-N-isopropylpiperazine-1-carboxamide (S3-4-I4-1-8)

Prepared according to the methods of S3-4-I4-1-1 from S3-1-14-1 and isopropyl isocyanate to provide the title compound as a formate salt. MS (ESI+) m/z: 712.5 [M+H]+; ¹H NMR (400 MHz, METHANOL-d4) δ=5.08 (br dd, 1H), 4.38 (d, 1H), 4.01 (d, 1H), 3.97-3.83 (m, 2H), 3.80-3.53 (m, 7H), 3.52-3.36 (m, 4H), 2.90 (s, 3H), 2.68 (br d, 1H), 2.50 (s, 3H), 2.44-2.27 (m, 7H), 2.11-2.02 (m, 1H), 1.85-1.66 (m, 3H), 1.47-1.04 (m, 28H), 0.86 (d, 3H).

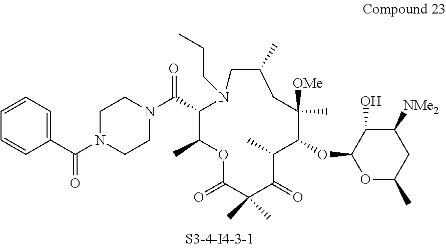

(2S,3R,6R,8R,9R,10R)-3-(4-benzoylpiperazine-1-carbonyl)-9-(((2S,3R,4S,6R)-4-(dimethylamino)-3-hydroxy-6-methyltetrahydro-2H-pyran-2-yl)oxy)-8-methoxy-2,6,8,10,12,12-hexamethyl-4-propyl-1-oxa-4-azacyclotridecane-11,13-dione (S3-4-I4-3-1)

Prepared according to the methods of S3-4-I4-1-1 from S3-1-I4-3 and benzoyl chloride to provide the title compound as a formate salt. MS (ESI+) m/z: 759.5 [M+H]+; ¹H NMR (400 MHz, METHANOL-d4) δ=7.57-7.37 (m, 5H), 4.37 (br d, 1H), 4.13-3.91 (m, 3H), 3.89-3.44 (m, 13H), 3.29-3.24 (m, 1H), 2.86 (br s, 3H), 2.81-2.61 (m, 3H), 2.48-2.35 (m, 7H), 1.99 (br d, 1H), 1.86-1.71 (m, 2H), 1.70-1.48 (m, 2H), 1.45-1.08 (m, 23H), 0.95-0.84 (m, 5H).

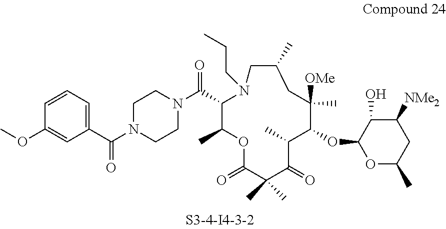

(2S,3R,6R,8R,9R,10R)-9-(((2S,3R,4S,6R)-4-(dimethylamino)-3-hydroxy-6-methyltetrahydro-2H-pyran-2-yl)oxy)-8-methoxy-3-(4-(3-methoxybenzoyl)piperazine-1-carbonyl)-2,6,8,10,12,12-hexamethyl-4-propyl-1-oxa-4-azacyclotridecane-11,13-dione (S3-4-I4-3-2)

Prepared according to the methods of S3-4-I4-1-1 from S3-1-I4-3 and 3-methoxybenzoyl chloride to provide the title compound as a formate salt. MS (ESI+) m/z: 789.5 [M+H]+; ¹H NMR (400 MHz, METHANOL-d4) δ=7.41 (t, 1H), 7.11-7.05 (m, 1H), 7.05-6.98 (m, 2H), 5.16-5.08 (m, 1H), 4.69-4.52 (m, 1H), 4.38 (d, 1H), 4.02 (br d, 1H), 3.95-3.81 (m, 5H), 3.80-3.45 (m, 9H), 3.30-3.25 (m, 1H), 2.98-2.63 (m, 6H), 2.40 (s, 8H), 2.31-2.10 (m, 1H), 1.98 (br s, 1H), 1.80 (br d, 1H), 1.72-1.61 (m, 1H), 1.53 (br s, 2H), 1.44-1.19 (m, 19H), 1.02-0.82 (m, 6H).

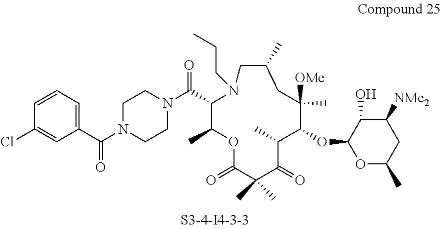

(2S,3R,6R,8R,9R,10R)-3-(4-(3-chlorobenzoyl)piperazine-1-carbonyl)-9-(((2S,3R,4S,6R)-4-(dimethylamino)-3-hydroxy-6-methyltetrahydro-2H-pyran-2-yl)oxy)-8-methoxy-2,6,8,10,12,12-hexamethyl-4-propyl-1-oxa-4-azacyclotridecane-11,13-dione (S3-4-I4-3-3)

Prepared according to the methods of S3-4-I4-1-1 from S3-1-I4-3 and 3-chlorobenzoyl chloride to provide the title compound as a formate salt. MS (ESI+) m/z: 793.4 [M+H]+; ¹H NMR (400 MHz, METHANOL-d4) δ=7.61-7.46 (m, 3H), 7.41 (br d, 1H), 5.11 (br d, 1H), 4.38 (br d, 1H), 4.17-4.05 (m, 1H), 4.03-3.40 (m, 12H), 3.29-3.23 (m, 1H), 2.88 (br s, 3H), 2.78-2.64 (m, 3H), 2.40 (s, 8H), 2.00 (br d, 1H), 1.79 (br d, 1H), 1.73-1.63 (m, 1H), 1.53 (br s, 3H), 1.45-0.98 (m, 23H), 0.96-0.78 (m, 6H).

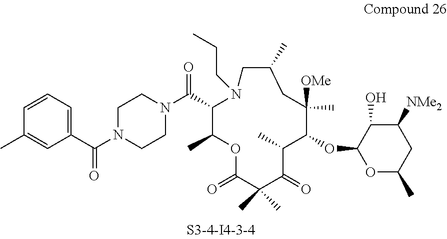

(2S,3R,6R,8R,9R,10R)-9-(((2S,3R,4S,6R)-4-(dimethylamino)-3-hydroxy-6-methyltetrahydro-2H-pyran-2-yl)oxy)-8-methoxy-2,6,8,10,12,12-hexamethyl-3-(4-(3-methylbenzoyl)piperazine-1-carbonyl)-4-propyl-1-oxa-4-azacyclotridecane-11,13-dione (S3-4-I4-3-4)

Prepared according to the methods of S3-4-I4-1-1 from S3-1-I4-3 and 3-methylbenzoyl chloride to provide the title compound as a formate salt. MS (ESI+) m/z: 773.5 [M+H]+; ¹H NMR (400 MHz, METHANOL-d4) δ=7.44-7.15 (m, 4H), 5.10 (br s, 1H), 4.35 (d, 1H), 4.19-3.93 (m, 2H), 3.91-3.40 (m, 11H), 3.25 (br dd, 1H), 2.85 (br s, 3H), 2.73-2.53 (m, 2H), 2.51-2.37 (m, 5H), 2.33 (s, 6H), 1.98 (br d, 1H), 1.84-1.59 (m, 3H), 1.58-1.33 (m, 8H), 1.33-1.08 (m, 15H), 0.97-0.80 (m, 6H).

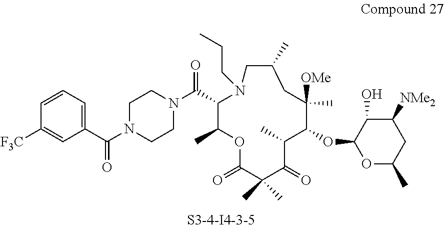

(2S,3R,6R,8R,9R,10R)-9-(((2S,3R,4S,6R)-4-(dimethylamino)-3-hydroxy-6-methyltetrahydro-2H-pyran-2-yl)oxy)-8-methoxy-2,6,8,10,12,12-hexamethyl-4-propyl-3-(4-(3-(trifluoromethyl)benzoyl)piperazine-1-carbonyl)-1-oxa-4-azacyclotridecane-11,13-dione (S3-4-I4-3-5)

Prepared according to the methods of S3-4-I4-1-1 from S3-1-I4-3 and 3-trifluoromethylbenzoyl chloride to provide the title compound as a formate salt. MS (ESI+) m/z: 827.5 [M+H]+; ¹H NMR (400 MHz, METHANOL-d4) δ=7.87-7.80 (m, 2H), 7.78-7.68 (m, 2H), 5.12 (br d, 1H), 4.38 (br d, 1H), 4.21-4.07 (m, 1H), 4.07-3.41 (m, 11H), 3.30-3.18 (m, 1H), 2.87 (br s, 3H), 2.77-2.65 (m, 2H), 2.63-2.26 (m, 8H), 2.01 (br d, 1H), 1.84-1.75 (m, 1H), 1.74-1.48 (m, 4H), 1.45-1.06 (m, 17H), 1.00-0.80 (m, 6H).

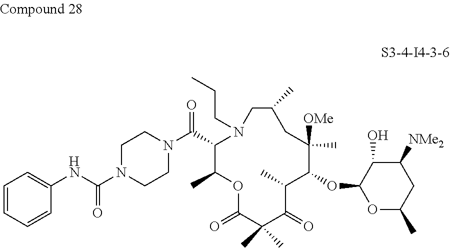

4-((2S,3R,6R,8R,9R,10R)-9-(((2S,3R,4S,6R)-4-(dimethylamino)-3-hydroxy-6-methyltetrahydro-2H-pyran-2-yl)oxy)-8-methoxy-2,6,8,10,12,12-hexamethyl-11,13-dioxo-4-propyl-1-oxa-4-azacyclotridecane-3-carbonyl)-N-phenylpiperazine-1-carboxamide (S3-4-I4-3-6)

Prepared according to the methods of S3-4-I4-1-1 from S3-1-14-3 and phenyl isocyanate to provide the title compound as a formate salt. MS (ESI+) m/z: 774.5 [M+H]+; ¹H NMR (400 MHz, METHANOL-d4) δ=8.66-8.48 (m, 0.3H), 7.41-7.35 (m, 2H), 7.29 (t, 2H), 7.05 (t, 1H), 5.14 (br s, 1H), 4.41 (d, 1H), 4.18-3.95 (m, 2H), 3.83-3.53 (m, 10H), 3.42-3.35 (m, 1H), 2.90 (s, 4H), 2.81-2.60 (m, 1H), 2.59-2.40 (m, 8H), 2.10-1.94 (m, 1H), 1.93-1.76 (m, 2H), 1.76-1.63 (m, 1H), 1.59-1.49 (m, 2H), 1.45-1.27 (m, 16H), 1.24-1.12 (m, 4H), 1.01-0.83 (m, 6H).

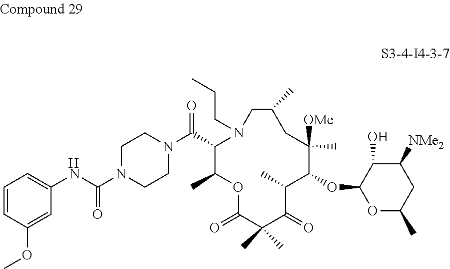

4-((2S,3R,6R,8R,9R,10R)-9-(((2S,3R,4S,6R)-4-(dimethylamino)-3-hydroxy-6-methyltetrahydro-2H-pyran-2-yl)oxy)-8-methoxy-2,6,8,10,12,12-hexamethyl-11,13-dioxo-4-propyl-1-oxa-4-azacyclotridecane-3-carbonyl)-N-(3-methoxyphenyl)piperazine-1-carboxamide (S3-4-I4-3-7)

Prepared according to the methods of S3-4-I4-1-1 from S3-1-I4-3 and 3-methoxyphenyl isocyanate to provide the title compound as a formate salt. MS (ESI+) m/z: 804.5 [M+H]+; ¹H NMR (400 MHz, METHANOL-d4) δ=6.70 (t, 1H), 6.59 (t, 1H), 6.46 (dd, 1H), 6.15 (dd, 1H), 4.67 (br s, 1H), 3.90 (d, 1H), 3.68-3.52 (m, 2H), 3.38-3.03 (m, 14H), 2.84-2.76 (m, 1H), 2.43 (s, 3H), 2.35-2.18 (m, 2H), 2.05-1.86 (m, 8H), 1.56 (br d, 1H), 1.39-1.28 (m, 2H), 1.26-1.16 (m, 1H), 1.12-1.02 (m, 2H), 0.94 (d, 6H), 0.88-0.76 (m, 10H), 0.75-0.62 (m, 4H), 0.52-0.35 (m, 6H).

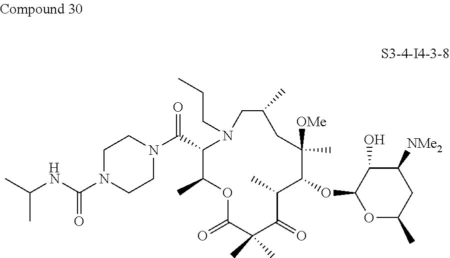

4-((2S,3R,6R,8R,9R,10R)-9-(((2S,3R,4S,6R)-4-(dimethylamino)-3-hydroxy-6-methyltetrahydro-2H-pyran-2-yl)oxy)-8-methoxy-2,6,8,10,12,12-hexamethyl-11,13-dioxo-4-propyl-1-oxa-4-azacyclotridecane-3-carbonyl)-N-isopropylpiperazine-1-carboxamide (S3-4-I4-3-8)

Prepared according to the methods of S3-4-I4-1-1 from S3-1-I4-3 and isopropyl isocyanate to provide the title compound as a formate salt. MS (ESI+) m/z: 740.5 [M+H]+; ¹H NMR (400 MHz, METHANOL-d4) δ=5.12 (br s, 1H), 4.36 (d, 1H), 4.13-3.98 (m, 2H), 3.89 (td, 1H), 3.77-3.54 (m, 7H), 3.47 (br d, 2H), 3.44-3.36 (m, 2H), 3.29-3.19 (m, 1H), 2.88 (s, 3H), 2.76-2.62 (m, 2H), 2.39-2.35 (m, 8H), 2.00 (br d, 1H), 1.93-1.81 (m, 1H), 1.79-1.59 (m, 3H), 1.56-1.43 (m, 3H), 1.32-1.20 (m, 12H), 1.19-1.08 (m, 10H), 0.99-0.80 (m, 7H).

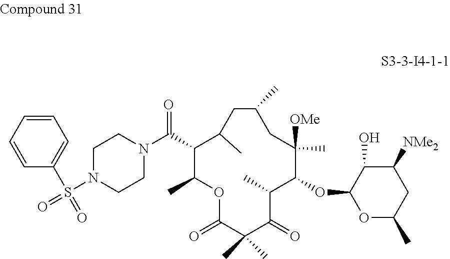

(2S,3R,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-3-(4-(phenylsulfonyl)piperazine-1-carbonyl)-1-oxa-4-azacyclotridecane-11,13-dione (S3-3-I4-1-1)

Prepared according to the methods of S3-4-I4-1-1 from S3-1-I4-1 and benzenesulfonyl chloride to provide the title compound as a formate salt. MS (ESI+) m/z: 767.4 [M+H]+; ¹H NMR (400 MHz, METHANOL-d4) δ=7.85-7.75 (m, 2H), 7.73-7.59 (m, 3H), 7.46-7.39 (m, 1H), 5.05-4.94 (m, 1H), 4.39 (d, 1H), 3.97 (br d, 1H), 3.89-3.41 (m, 8H), 3.14-2.90 (m, 5H), 2.87 (s, 3H), 2.50 (br s, 5H), 2.42 (s, 3H), 2.35-2.12 (m, 1H), 1.85 (br t, 2H), 1.77-1.54 (m, 2H), 1.44-1.17 (m, 16H), 1.12-0.96 (m, 4H), 0.75 (br d, 3H).

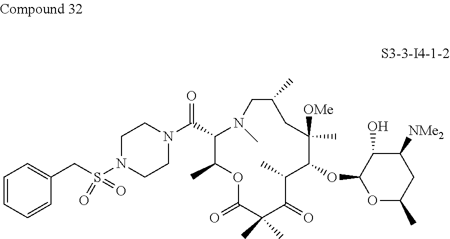

(2S,3R,6R,8R,9R,10R)-3-(4-(benzylsulfonyl)piperazine-1-carbonyl)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-1-oxa-4-azacyclotridecane-11,13-dione (S3-3-I4-1-2)

Prepared according to the methods of S3-4-I4-1-1 from S3-1-I4-1 and phenylmethanesulfonyl chloride to provide the title compound as a formate salt. MS (ESI+) m/z: 781.4 [M+H]+; ¹H NMR (400 MHz, METHANOL-d4) δ=7.45 (br dd, 2H), 7.40-7.33 (m, 3H), 5.03 (br d, 1H), 4.46-4.32 (m, 3H), 3.98 (d, 1H), 3.85 (br d, 1H), 3.73-3.49 (m, 7H), 3.24-3.02 (m, 4H), 2.87 (s, 3H), 2.75 (m, 1H), 2.50-2.20 (m, 10H), 1.98 (br d, 1H), 1.84-1.64 (m, 3H), 1.45-1.20 (m, 17H), 1.17-1.06 (m, 4H), 0.85 (d, 3H).

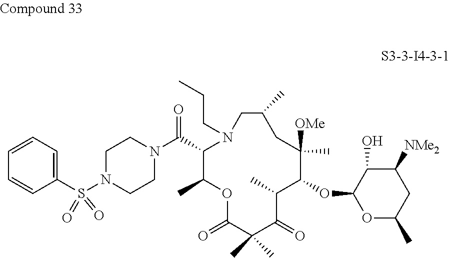

(2S,3R,6R,8R,9R,10R)-9-(((2S,3R,4S,6R)-4-(dimethylamino)-3-hydroxy-6-methyltetrahydro-2H-pyran-2-yl)oxy)-8-methoxy-2,6,8,10,12,12-hexamethyl-3-(4-(phenylsulfonyl)piperazine-1-carbonyl)-4-propyl-1-oxa-4-azacyclotridecane-11,13-dione (S3-3-I4-3-1)

Prepared according to the methods of S3-4-I4-1-1 from S3-1-I4-3 and benzenesulfonyl chloride to provide the title compound as a formate salt. MS (ESI+) m/z: 795.5 [M+H]+; ¹H NMR (400 MHz, METHANOL-d4) δ=8.57 (br s, 0.22H), 7.86-7.78 (m, 2H), 7.76-7.69 (m, 1H), 7.68-7.58 (m, 2H), 5.06 (br s, 1H), 4.37 (d, 1H), 4.11-3.92 (m, 2H), 3.87-3.68 (m, 4H), 3.66-3.48 (m, 3H), 3.29-3.23 (m, 1H), 3.04 (br d, 4H), 2.87 (s, 3H), 2.80-2.54 (m, 2H), 2.41 (s, 6H), 2.37-2.21 (m, 2H), 1.96-1.68 (m, 3H), 1.57 (br s, 1H), 1.52-1.34 (m, 9H), 1.33-1.21 (m, 10H), 1.16-1.08 (m, 1H), 1.04 (d, 3H), 0.87 (t, 3H), 0.78 (d, 3H).

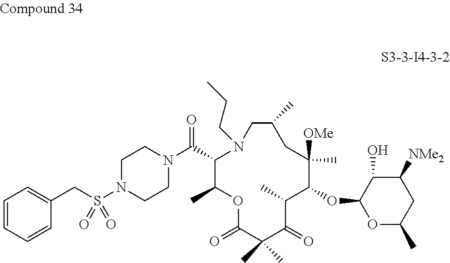

(2S,3R,6R,8R,9R,10R)-3-(4-(benzylsulfonyl)piperazine-1-carbonyl)-9-(((2S,3R,4S,6R)-4-(dimethylamino)-3-hydroxy-6-methyltetrahydro-2H-pyran-2-yl)oxy)-8-methoxy-2,6,8,10,12,12-hexamethyl-4-propyl-1-oxa-4-azacyclotridecane-11,13-dione (S3-3-I4-3-2)

Prepared according to the methods of S3-4-I4-1-1 from S3-1-I4-3 and phenylmethanesulfonyl chloride to provide the title compound as a formate salt. MS (ESI+) m/z: 809.5 [M+H]+; ¹H NMR (400 MHz, METHANOL-d4) δ=8.55 (br s, 0.24H), 7.51-7.43 (m, 2H), 7.42-7.34 (m, 3H), 5.07 (br s, 1H), 4.42 (s, 2H), 4.37 (d, 1H), 4.07-3.93 (m, 2H), 3.75-3.50 (m, 6H), 3.29-3.26 (m, 1H), 3.24-3.04 (m, 4H), 2.84 (s, 3H), 2.77 (br t, 1H), 2.69-2.56 (m, 1H), 2.42 (s, 6H), 2.37-2.28 (m, 1H), 1.93 (br d, 1H), 1.86-1.66 (m, 2H), 1.62 (br s, 1H), 1.53-1.43 (m, 2H), 1.40 (s, 3H), 1.36 (s, 3H), 1.30 (br d, 4H), 1.28-1.22 (m, 6H), 1.14-1.09 (d, 4H), 0.93-0.81 (m, 6H).

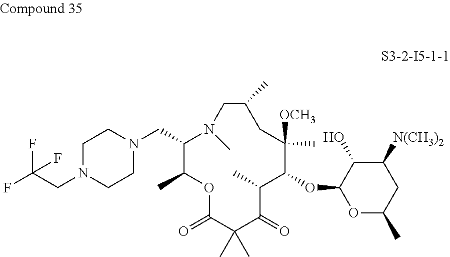 [SMILES: CO[C@]1(C)C[C@@H](C)CN(C)[C@@H](CN2CCN(CC(F)(F)F)CC2)[C@H](C)OC(=O)C(C)(C)C(=O)[C@H](C)[C@H]1O[C@@H]1O[C@H](C)C[C@H](N(C)C)[C@H]1O]

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-3-((4-(2,2,2-trifluoroethyl)piperazin-1-yl)methyl)-1-oxa-4-azacyclotridecane-11,13-dione (S3-2-I5-1-1)

S3-1-I5-1 (36.8 mg, 0.051 mmol) was dissolved in dry THE (0.6 mL) under nitrogen. Phenylsilane (12.5 μL, 1.020 mmol) was added followed by trifluoroacetic acid (6.8 μL, 0.090 mmol). The reaction mixture was placed in a pre-heated dry block at 70° C. and stirred for 6 h. The reaction was cooled, quenched through the addition of sat. NaHCO₃(1.5 mL) and extracted with EtOAc (1 mL×3). The combined extracts were dried over Na2SO4, were filtered, and were concentrated. The resulting crude material was dissolved in MeOH (1 mL), was heated at 40° C. overnight, and was concentrated. The residue was purified by HPLC (Atlantis T3 column, 5-50% MeCN-water-0.1% HCO2H) to give 8.31 mg of the title compound as a formate salt. MS (ESI+) m/z: 695.33 [M+H]+; 1H NMR (400 MHz, Methanol-d) δ 8.40 (s, 3H), 5.41 (dt, 1H), 4.50 (d, 1H), 4.11 (d, 1H), 3.91-3.80 (m, 1H), 3.73 (ddd, 1H), 3.57-3.47 (m, 1H), 3.47-3.36 (m, 2H), 3.18 (s, 3H), 3.07 (q, 3H), 3.02 (s, 3H), 2.95-2.84 (m, 2H), 2.82 (s, 6H), 2.77-2.61 (m, 6H), 2.61-2.51 (m, 2H), 2.27 (d, 1H), 2.08-1.99 (m, 1H), 1.83 (d, 1H), 1.58-1.47 (m, 4H), 1.42-1.30 (m, 17H), 1.07 (d, 3H).

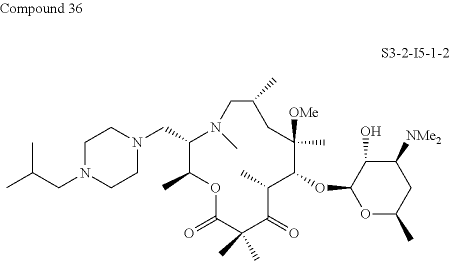

(2S,3S,6R,8R,9R,10R)-9-(((2S,3R,4S,6R)-4-(dimethylamino)-3-hydroxy-6-methyltetrahydro-2H-pyran-2-yl)oxy)-3-((4-isobutylpiperazin-1-yl)methyl)-8-methoxy-2,4,6,8,10,12,12-heptamethyl-1-oxa-4-azacyclotridecane-11,13-dione (S3-2-I5-1-2)

Prepared according to the methods of S3-2-I5-1-1, substituting isobutyraldehyde to provide the title compound as a formate salt. MS (ESI+) m/z: 669.4 [M+H]+; ¹H NMR (400 MHz, METHANOL-d4) δ=8.57 (s, 1H), 5.44 (br dd, 1H), 4.42 (dd, 1H), 4.26-3.99 (m, 1H), 3.98-3.74 (m, 1H), 3.65-3.38 (m, 3H), 3.31-3.19 (m, 2H), 3.15-3.00 (m, 2H), 2.96 (s, 2H), 2.94-2.74 (m, 2H), 2.71-2.42 (m, 8H), 2.42-2.24 (m, 9H), 2.21-2.11 (m, 3H), 1.92-1.65 (m, 4H), 1.59-1.48 (m, 2H), 1.46-1.18 (m, 17H), 1.10 (br d, 1H), 1.00-0.90 (m, 7H).

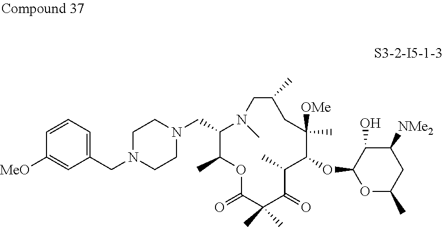

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-3-((4-(3-methoxybenzyl)piperazin-1-yl)methyl)-2,4,6,8,10,12,12-heptamethyl-1-oxa-4-azacyclotridecane-11,13-dione (S3-2-I5-1-3)

Prepared according to the methods of S3-2-15-1-1, substituting 3-methoxybenzaldehyde to provide the title compound as a formate salt. MS (ESI+) m/z: 733.5 [M+H]+; ¹H NMR (400 MHz, METHANOL-d₄) δ=8.55 (s, 0.3H), 7.22 (t, 1H), 6.95-6.86 (m, 2H), 6.83 (br d, 1H), 5.41 (br dd, 1H), 4.50-4.34 (m, 1H), 4.12-4.00 (m, 1H), 3.91-3.73 (m, 4H), 3.65-3.39 (m, 5H), 3.29-3.13 (m, 2H), 3.01 (s, 1H), 2.96-2.91 (m, 2H), 2.90-2.76 (m, 1H), 2.71-2.40 (m, 10H), 2.39-2.21 (m, 10H), 2.20-2.08 (m, 1H), 1.89-1.63 (m, 3H), 1.50 (s, 1H), 1.46-1.32 (m, 9H), 1.31-1.16 (m, 11H), 1.15-1.04 (m, 1H), 0.89 (br d, 2H).

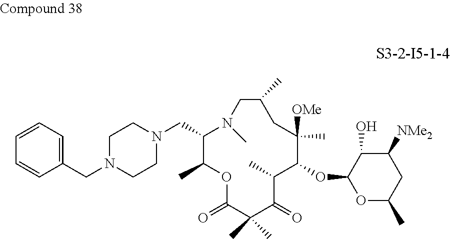

(2S,3S,6R,8R,9R,10R)-3-((4-benzylpiperazin-1-yl)methyl)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-1-oxa-4-azacyclotridecane-11,13-dione (S3-2-I5-1-4)

Prepared according to the methods of S3-2-I5-1-1, substituting benzaldehyde to provide the title compound as a formate salt. MS (ESI+) m/z: 703.5 [M+H]+; ¹H NMR (400 MHz, METHANOL-d4) δ=7.40-7.15 (m, 5H), 4.40 (br d, 1H), 4.15-4.01 (m, 1H), 3.91-3.78 (m, 1H), 3.65-3.46 (m, 4H), 3.28-3.14 (m, 2H), 3.10-2.75 (m, 6H), 2.70-2.09 (m, 21H), 1.93-1.62 (m, 3H), 1.55-1.47 (m, 1H), 1.44-1.16 (m, 19H), 1.11-1.00 (m, 2H), 0.96-0.82 (m, 2H).

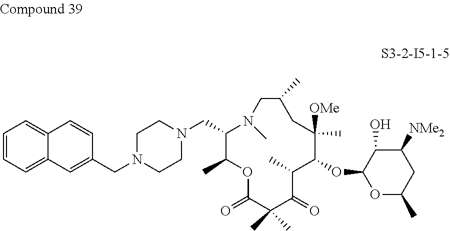

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-3-((4-(naphthalen-2-ylmethyl)piperazin-1-yl)methyl)-1-oxa-4-azacyclotridecane-11,13-dione (S3-2-I5-1-5)

Prepared according to the methods of S3-2-I5-1-1, substituting 2-naphthaldehyde to provide the title compound as a formate salt. MS (ESI+) m/z: 753.5 [M+H]+; ¹H NMR (400 MHz, METHANOL-d4) δ=8.63-8.48 (m, 1H), 7.85-7.75 (m, 4H), 7.51-7.43 (m, 3H), 5.52-5.31 (m, 1H), 4.39 (d, 1H), 4.11-4.03 (m, 1H), 3.91-3.77 (m, 1H), 3.72-3.45 (m, 5H), 3.29-3.12 (m, 2H), 3.10-2.97 (m, 1H), 2.94-2.75 (m, 4H), 2.72-2.51 (m, 6H), 2.45 (br dd, 3H), 2.38-2.20 (m, 11H), 2.18-2.10 (m, 1H), 1.89-1.66 (m, 3H), 1.65-1.47 (m, 1H), 1.44-1.31 (m, 8H), 1.29-1.02 (m, 14H), 0.88 (d, 2H).

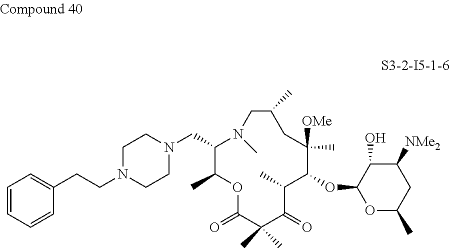

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-3-((4-phenethylpiperazin-1-yl)methyl)-1-oxa-4-azacyclotridecane-11,13-dione (S3-2-I5-1-6)

Prepared according to the methods of S3-2-I5-1-1, substituting phenylacetaldehyde to provide the title compound as a formate salt. MS (ESI+) m/z: 717.5 [M+H]+; ¹H NMR (400 MHz, METHANOL-d4) δ=8.61-8.47 (m, 1H), 7.29-7.15 (m, 5H), 4.40 (d, 1H), 4.12-4.03 (m, 1H), 3.64-3.45 (m, 3H), 3.28-3.17 (m, 1H), 3.15-2.96 (m, 1H), 2.94 (s, 3H), 2.85-2.75 (m, 3H), 2.70-2.53 (m, 9H), 2.47 (br dd, 3H), 2.38 (s, 3H), 2.36-2.29 (m, 8H), 2.25-2.06 (m, 1H), 1.94-1.59 (m, 3H), 1.54-1.33 (m, 8H), 1.31-1.03 (m, 15H), 0.90 (d, 3H).

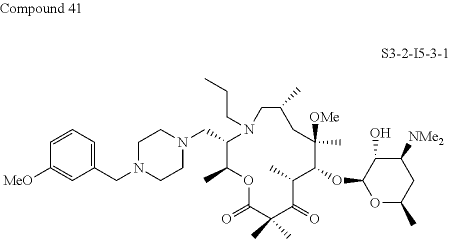

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-3-((4-(3-methoxybenzyl)piperazin-1-yl)methyl)-2,6,8,10,12,12-hexamethyl-4-propyl-1-oxa-4-azacyclotridecane-11,13-dione (S3-2-I5-3-1)

Prepared according to the methods of S3-2-I5-1-1, from S3-1-I5-3 and substituting 3-methoxybenzaldehyde to provide the title compound as a formate salt. MS (ESI+) m/z: 761.5 [M+H]+; ¹H NMR (400 MHz, METHANOL-d4) δ=8.68-8.48 (m, 0.3H), 7.24 (t, 1H), 6.95-6.82 (m, 3H), 4.50-4.36 (m, 1H), 4.19-4.06 (m, 1H), 4.05-3.92 (m, 1H), 3.81 (s, 3H), 3.65-3.38 (m, 5H), 3.29-3.08 (m, 1H), 3.04-2.88 (m, 4H), 2.83-2.66 (m, 3H), 2.62-2.34 (m, 17H), 2.28-2.07 (m, 2H), 1.93-1.72 (m, 2H), 1.67-1.59 (m, 1H), 1.51-1.17 (m, 23H), 1.17-1.04 (m, 2H), 0.96-0.83 (m, 5H).

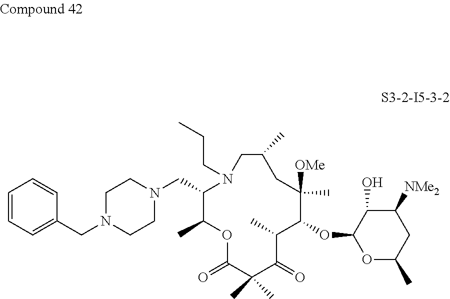

(2S,3S,6R,8R,9R,10R)-3-((4-benzylpiperazin-1-yl)methyl)-9-(((2S,3R,4S,6R)-4-(dimethylamino)-3-hydroxy-6-methyltetrahydro-2H-pyran-2-yl)oxy)-8-methoxy-2,6,8,10,12,12-hexamethyl-4-propyl-1-oxa-4-azacyclotridecane-11,13-dione (S3-2-I5-3-2)

Prepared according to the methods of S3-2-I5-1-1, from S3-1-I5-3 and substituting benzaldehyde to provide the title compound as a formate salt. MS (ESI+) m/z: 731.5 [M+H]+; ¹H NMR (400 MHz, METHANOL-d4) δ=7.37-7.19 (m, 5H), 4.40 (br d, 1H), 4.08 (br d, 1H), 3.62-3.43 (m, 4H), 3.00-2.86 (m, 3H), 2.82-2.01 (m, 23H), 1.82-0.96 (m, 27H), 0.93-0.71 (m, 6H).

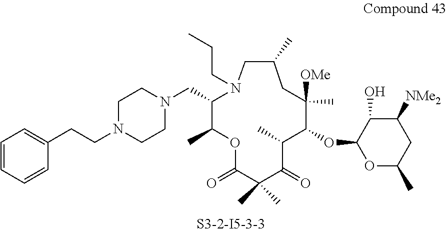

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,6,8,10,12,12-hexamethyl-3-((4-phenethylpiperazin-1-yl)methyl)-4-propyl-1-oxa-4-azacyclotridecane-11,13-dione (S3-2-I5-3-3)

Prepared according to the methods of S3-2-I5-1-1, from S3-1-I5-3 and substituting phenylacetaldehyde to provide the title compound as a formate salt. MS (ESI+) m/z: 745.6 [M+H]+; ¹H NMR (400 MHz, METHANOL-d4) δ=7.33-7.12 (m, 5H), 4.40 (br d, 1H), 4.08 (br d, 1H), 3.64-3.45 (m, 2H), 2.94 (s, 3H), 2.85-2.08 (m, 27H), 1.82-0.97 (m, 27H), 0.95-0.75 (m, 6H).

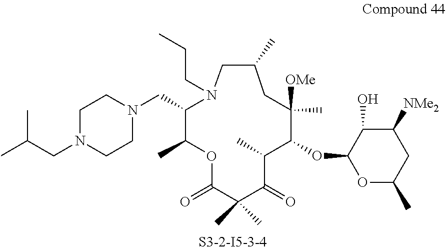

(2S,3S,6R,8R,9R,10R)-9-(((2S,3R,4S,6R)-4-(dimethylamino)-3-hydroxy-6-methyltetrahydro-2H-pyran-2-yl)oxy)-3-((4-isobutylpiperazin-1-yl)methyl)-8-methoxy-2,6,8,10,12,12-hexamethyl-4-propyl-1-oxa-4-azacyclotridecane-11,13-dione (S3-2-I5-3-4)

Prepared according to the methods of S3-2-15-1-1, from S3-1-15-3 and substituting isobutyraldehyde to provide the title compound as a formate salt. MS (ESI+) m/z: 697.6 [M+H]+; ¹H NMR (400 MHz, METHANOL-d4) δ=4.82-4.61 (m, 1H), 4.40 (br d, 1H), 4.08 (br d, 1H), 3.66-3.45 (m, 2H), 3.30-3.12 (m, 2H), 3.03-2.86 (m, 3H), 2.85-2.73 (m, 1H), 2.72-2.60 (m, 2H), 2.60-2.26 (m, 15H), 2.25-1.98 (m, 4H), 1.89-1.54 (m, 4H), 1.53-1.33 (m, 8H), 1.32-1.02 (m, 13H), 0.99-0.68 (m, 10H).

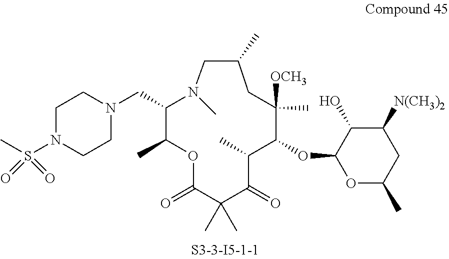 [SMILES: CO[C@]1(C)C[C@@H](C)CN(C)[C@@H](CN2CCN(S(C)(=O)=O)CC2)[C@H](C)OC(=O)C(C)(C)C(=O)[C@H](C)[C@H]1O[C@@H]1O[C@H](C)C[C@H](N(C)C)[C@H]1O]

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-3-((4-(methylsulfonyl)piperazin-1-yl)methyl)-1-oxa-4-azacyclotridecane-11,13-dione (S3-3-I5-1-1)

S3-1-I5-1 (37.4 mg, 0.0521 mmol) and 4-dimethylaminopyridine (1 mg, 0.008 mmol) were dissolved in dichloromethane (0.45 mL) and N,N-diisopropylethylamine (0.050 mL, 0.26 mmol). The solution was cooled to 0° C., methanesulfonyl chloride (0.012 mL, 0.156 mmol) was added, and the reaction mixture was allowed to warm to rt. After 3 h, the reaction was quenched through the addition of sat. NaHCO₃(1 mL) and was extracted with EtOAc (1 mL×3). The combined extracts were dried over Na2SO4, were filtered, and were concentrated. The resulting crude material was dissolved in MeOH (1 mL), was heated at 40° C. overnight, and was concentrated. The residue was purified by HPLC (Atlantis T3 column, 5-50% MeCN-water-0.1% HCO2H) to give 14.1 mg of the title compound as a formate salt. MS (ESI+) m/z: 691.30 [M+H]+; 1H NMR (400 MHz, Methanol-d) δ 8.33 (s, 3H), 5.44 (dq, 1H), 4.49 (d, 1H), 4.10 (d, 1H), 3.90-3.80 (m, 1H), 3.72 (ddd, 1H), 3.55-3.37 (m, 3H), 3.27-3.19 (m, 4H), 3.16 (s, 3H), 3.10-3.02 (m, 1H), 3.02 (s, 3H), 2.95-2.85 (m, 2H), 2.84 (s, 3H), 2.81 (s, 6H), 2.74-2.59 (m, 5H), 2.31-2.21 (m, 1H), 2.07-1.99 (m, 1H), 1.82 (d, 1H), 1.57-1.43 (m, 4H), 1.42-1.33 (m, 13H), 1.31 (d, 3H), 1.06 (d, 3H).

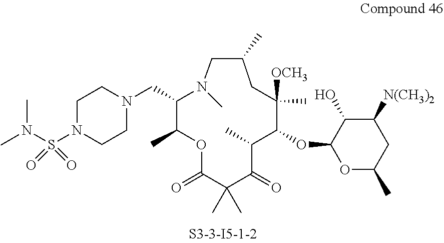 [SMILES: CO[C@]1(C)C[C@@H](C)CN(C)[C@@H](CN2CCN(S(=O)(=O)N(C)C)CC2)[C@H](C)OC(=O)C(C)(C)C(=O)[C@H](C)[C@H]1O[C@@H]1O[C@H](C)C[C@H](N(C)C)[C@H]1O]

4-(((2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-11,13-dioxo-1-oxa-4-azacyclotridecan-3-yl)methyl)-N,N-dimethylpiperazine-1-sulfonamide (S3-3-I5-1-2)

Prepared according to the methods of S3-3-I5-1-1 from S3-1-I5-1 and dimethylsulfamoyl chloride to provide the title compound as a formate salt. MS (ESI+) m/z: 720.24 [M+H]+; 1H NMR (400 MHz, Methanol-d) δ 8.25 (s, 1H), 5.45 (dq, 1H), 4.51 (d, 1H), 4.11 (d, 1H), 3.93-3.85 (m, 1H), 3.79-3.70 (m, 1H), 3.58-3.40 (m, 3H), 3.29-3.23 (m, 3H), 3.19 (s, 3H), 3.12-2.99 (m, 4H), 2.94 (dd, 1H), 2.89 (s, 3H), 2.87-2.81 (m, 9H), 2.68 (q, 1H), 2.64 (s, 6H), 2.63-2.56 (m, 2H), 2.37-2.20 (m, 1H), 2.10-2.02 (m, 1H), 1.84 (d, 1H), 1.61-1.47 (m, 4H), 1.44-1.26 (m, 15H), 1.08 (d, 3H).

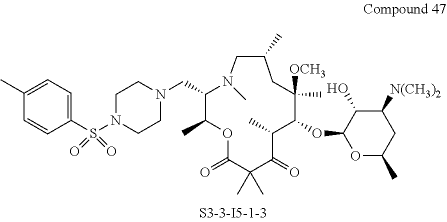 [SMILES: CO[C@]1(C)C[C@@H](C)CN(C)[C@@H](CN2CCN(S(=O)(=O)c3ccc(C)cc3)CC2)[C@H](C)OC(=O)C(C)(C)C(=O)[C@H](C)[C@H]1O[C@@H]1O[C@H](C)C[C@H](N(C)C)[C@H]1O]

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-3-((4-tosylpiperazin-1-yl)methyl)-1-oxa-4-azacyclotridecane-11,13-dione (S3-3-I5-1-3)

Prepared according to the methods of S3-3-I5-1-1 from S3-1-I5-1 and p-toluenesulfonyl chloride to provide the title compound as a formate salt. MS (ESI+) m/z: 767.38 [M+H]+; 1H NMR (400 MHz, Methanol-d) δ 8.36 (s, 3H), 7.63 (d, 2H), 7.41 (d, 2H), 5.38 (dt, 1H), 4.46 (d, 1H), 4.06 (d, 1H), 3.78 (ddd, 1H), 3.74-3.66 (m, 1H), 3.51-3.35 (m, 3H), 3.03 (s, 3H), 2.99 (d, 5H), 2.96 (s, 3H), 2.88-2.82 (m, 2H), 2.80 (s, 6H), 2.71-2.62 (m, 2H), 2.62-2.52 (m, 3H), 2.42 (d, 3H), 2.25-2.14 (m, 1H), 2.04-1.97 (m, 1H), 1.78 (d, 1H), 1.55-1.41 (m, 5H), 1.37-1.24 (m, 16H), 1.00 (d, 3H).

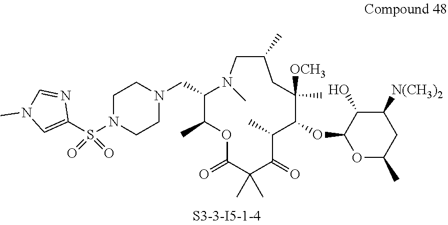 [SMILES: CO[C@]1(C)C[C@@H](C)CN(C)[C@@H](CN2CCN(S(=O)(=O)c3cn(C)cn3)CC2)[C@H](C)OC(=O)C(C)(C)C(=O)[C@H](C)[C@H]1O[C@@H]1O[C@H](C)C[C@H](N(C)C)[C@H]1O]

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-3-((4-((1-methyl-1H-imidazol-4-yl)sulfonyl)piperazin-1-yl)methyl)-1-oxa-4-azacyclotridecane-11,13-dione (S3-3-I5-1-4)

Prepared according to the methods of S3-3-I5-1-1 from S3-1-I5-1 and 1-methyl-1H-imidazole-4-sulfonyl chloride to provide the title compound as a formate salt. MS (ESI+) m/z: 757.32 [M+H]+; 1H NMR (400 MHz, Methanol-d)δ 8.35 (s, 3H), 7.76 (d, 1H), 7.71 (d, 1H), 5.39 (dt, 1H), 4.47 (d, 1H), 4.07 (d, 1H), 3.85-3.78 (m, 1H), 3.77 (s, 3H), 3.71 (ddd, 1H), 3.52-3.45 (m, 1H), 3.44-3.35 (m, 2H), 3.19-3.10 (m, 4H), 3.08 (s, 3H), 3.04-2.93 (m, 4H), 2.92-2.82 (m, 2H), 2.80 (s, 6H), 2.70-2.53 (m, 5H), 2.28-2.14 (m, 1H), 2.06-1.98 (m, 1H), 1.79 (d, 1H), 1.59-1.41 (m, 4H), 1.39-1.25 (m, 17H), 1.02 (d, 3H).

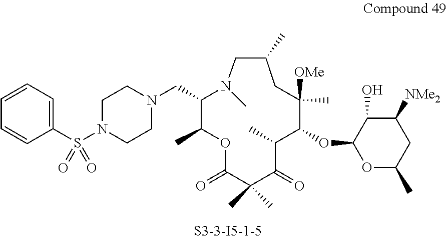

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-3-((4-(phenylsulfonyl)piperazin-1-yl)methyl)-1-oxa-4-azacyclotridecane-11,13-dione (S3-3-I5-1-5)

Prepared according to the methods of S3-3-I5-1-1 from S3-1-I5-1 and benzenesulfonyl chloride to provide the title compound as a formate salt. MS (ESI+) m/z: 753.4 [M+H]+; ¹H NMR (400 MHz, METHANOL-d₄) δ=8.56 (br s, 0.16H), 7.86-7.74 (m, 2H), 7.73-7.66 (m, 1H), 7.66-7.59 (m, 2H), 7.47-7.38 (m, 1H), 5.39 (br dd, 1H), 4.39 (br d, 1H), 4.14-3.95 (m, 1H), 3.88-3.69 (m, 1H), 3.66-3.39 (m, 2H), 3.28-3.10 (m, 1H), 3.09-2.94 (m, 6H), 2.91 (s, 2H), 2.87-2.41 (m, 8H), 2.34 (br d, 8H), 2.24-2.05 (m, 2H), 1.88-1.57 (m, 2H), 1.48 (s, 1H), 1.42-1.19 (m, 16H), 1.14 (br d, 2H), 1.04 (br d, 1H), 0.94-0.72 (m, 2H).

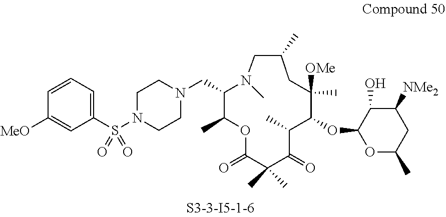

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-3-((4-((3-methoxyphenyl)sulfonyl)piperazin-1-yl)methyl)-2,4,6,8,10,12,12-heptamethyl-1-oxa-4-azacyclotridecane-11,13-dione (S3-3-I5-1-6)

Prepared according to the methods of S3-3-I5-1-1 from S3-1-I5-1 and 3-methoxybenzenesulfonyl chloride to provide the title compound as a formate salt. MS (ESI+) m/z: 783.4 [M+H]+; ¹H NMR (400 MHz, METHANOL-d4) δ=8.55 (s, 0.17H), 7.59-7.48 (m, 1H), 7.33 (br d, 1H), 7.28-7.21 (m, 2H), 5.39 (br dd, 1H), 4.73-4.45 (m, 1H), 4.39 (d, 1H), 4.08-3.98 (m, 1H), 3.88 (s, 3H), 3.63-3.43 (m, 2H), 3.29-3.21 (m, 1H), 3.11-2.94 (m, 5H), 2.91 (s, 2H), 2.87-2.73 (m, 1H), 2.71-2.41 (m, 7H), 2.40-2.25 (m, 10H), 2.23-2.05 (m, 2H), 1.74 (br d, 1H), 1.65 (br s, 1H), 1.45-1.00 (m, 21H), 0.85 (d, 3H).

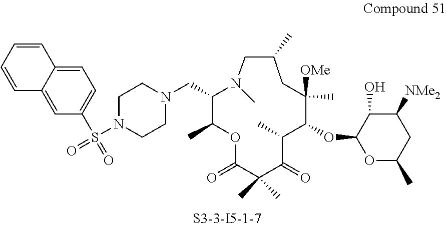

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-3-((4-(naphthalen-2-ylsulfonyl)piperazin-1-yl)methyl)-1-oxa-4-azacyclotridecane-11,13-dione (S3-3-I5-1-7)

Prepared according to the methods of S3-3-15-1-1 from S3-1-15-1 and 3-naphthalene-2-sulfonyl chloride to provide the title compound as a formate salt. MS (ESI+) m/z: 803.4 [M+H]+; ¹H NMR (400 MHz, METHANOL-d4) δ=8.56 (br s, 1H), 8.39 (br s, 1H), 8.09 (dd, 2H), 8.02 (d, 1H), 7.81-7.75 (m, 1H), 7.75-7.64 (m, 2H), 5.37 (br dd, 1H), 4.58 (br s, 1H), 4.44-4.33 (m, 1H), 4.09-4.01 (m, 1H), 3.83-3.74 (m, 1H), 3.58 (br dd, 1H), 3.52-3.34 (m, 1H), 3.25-3.18 (m, 1H), 3.18-3.03 (m, 4H), 3.03-2.77 (m, 8H), 2.77-2.53 (m, 6H), 2.52-2.38 (m, 4H), 2.35 (s, 4H), 2.32-2.21 (m, 2H), 2.21-2.03 (m, 1H), 1.85-1.71 (m, 2H), 1.69-1.54 (m, 1H), 1.47 (s, 2H), 1.40-1.19 (m, 18H), 1.16-1.10 (m, 1H), 1.05-0.97 (m, 2H), 0.85-0.78 (m, 1H).

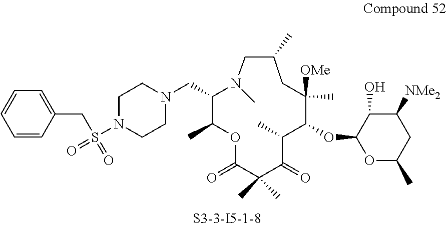

(2S,3S,6R,8R,9R,10R)-3-((4-(benzylsulfonyl)piperazin-1-yl)methyl)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-1-oxa-4-azacyclotridecane-11,13-dione (S3-3-I5-1-8)

Prepared according to the methods of S3-3-15-1-1 from S3-1-15-1 and phenylmethanesulfonyl chloride to provide the title compound as a formate salt. MS (ESI+) m/z: 767.4 [M+H]+; ¹H NMR (400 MHz, METHANOL-d4) δ=7.48-7.35 (m, 5H), 4.70-4.50 (m, 2H), 4.45-4.32 (m, 3H), 4.10-4.06 (m, 2H), 3.88-3.77 (m, 1H), 3.69-3.45 (m, 3H), 3.31-3.11 (m, 7H), 3.03 (s, 2H), 2.95 (s, 3H), 2.82-2.56 (m, 4H), 2.55-2.30 (m, 14H), 2.29-2.10 (m, 2H), 1.93-1.65 (m, 3H), 1.65-1.48 (m, 2H), 1.47-1.20 (m, 19H), 1.10 (br d, 1H), 0.92 (br d, 2H).

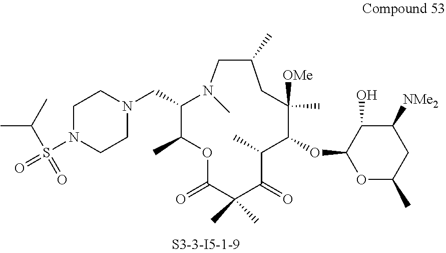

(2S,3S,6R,8R,9R,10R)-9-(((2S,3R,4S,6R)-4-(dimethylamino)-3-hydroxy-6-methyltetrahydro-2H-pyran-2-yl)oxy)-3-((4-(isopropylsulfonyl)piperazin-1-yl)methyl)-8-methoxy-2,4,6,8,10,12,12-heptamethyl-1-oxa-4-azacyclotridecane-11,13-dione (S3-3-I5-1-9)

Prepared according to the methods of S3-3-15-1-1 from S3-1-15-1 and 2-propanesulfonyl chloride to provide the title compound as a formate salt. MS (ESI+) m/z: 719.5 [M+H]+; ¹H NMR (400 MHz, METHANOL-d4) δ=5.56-5.38 (m, 1H), 4.77-4.58 (m, 3H), 4.41 (br d, 3H), 4.17-3.96 (m, 2H), 3.93-3.78 (m, 1H), 3.69-3.41 (m, 7H), 3.28-3.09 (m, 4H), 3.02 (s, 3H), 3.00-2.90 (m, 2H), 2.67-2.47 (m, 7H), 2.44-2.31 (m, 10H), 2.16 (s, 3H), 1.89-1.61 (m, 4H), 1.57-1.21 (m, 30H), 1.19-1.05 (m, 2H), 1.02-0.86 (m, 3H).

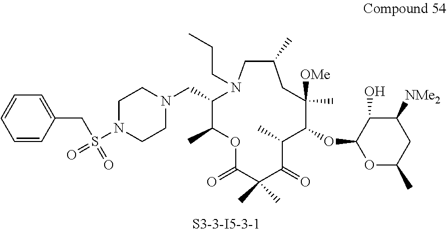

(2S,3S,6R,8R,9R,10R)-3-((4-(benzylsulfonyl)piperazin-1-yl)methyl)-9-(((2S,3R,4S,6R)-4-(dimethylamino)-3-hydroxy-6-methyltetrahydro-2H-pyran-2-yl)oxy)-8-methoxy-2,6,8,10,12,12-hexamethyl-4-propyl-1-oxa-4-azacyclotridecane-11,13-dione (S3-3-I5-3-1)

Prepared according to the methods of S3-3-I5-1-1 from S3-1-I5-3 and phenylmethanesulfonyl chloride to provide the title compound as a formate salt. MS (ESI+) m/z: 795.5 [M+H]+; ¹H NMR (400 MHz, METHANOL-d4) δ=7.48-7.33 (m, 4H), 7.32-7.16 (m, 1H), 4.95-4.82 (m, 1H), 4.47-4.29 (m, 3H), 4.18-3.86 (m, 2H), 3.66-3.54 (m, 1H), 3.53-3.40 (m, 1H), 3.39-3.31 (m, 1H), 3.18-3.06 (m, 4H), 3.01-2.81 (m, 4H), 2.76 (br d, 1H), 2.67-1.90 (m, 14H), 1.86-1.75 (m, 1H), 1.66 (br d, 1H), 1.59 (dt, 1H), 1.53-1.15 (m, 19H), 1.14-1.01 (m, 1H), 0.93-0.79 (m, 4H).

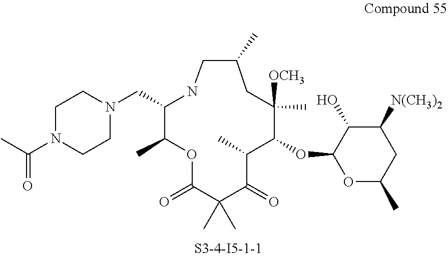 [SMILES: CO[C@]1(C)C[C@@H](C)CN[C@@H](CN2CCN(C(C)=O)CC2)[C@H](C)OC(=O)C(C)(C)C(=O)[C@H](C)[C@H]1O[C@@H]1O[C@H](C)C[C@H](N(C)C)[C@H]1O]

(2S,3S,6R,8R,9R,10R)-3-((4-acetylpiperazin-1-yl)methyl)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-1-oxa-4-azacyclotridecane-11,13-dione (S3-4-I5-1-1)

S3-1-15-1 (36.0 mg, 0.0502 mmol) and 4-dimethylaminopyridine (1 mg, 0.008 mmol) were dissolved in dichloromethane (0.45 mL) and N,N-diisopropylethylamine (0.050 mL, 0.26 mmol). The solution was cooled to 0° C., acetyl chloride (0.0106 mL, 0.150 mmol) was added, and the reaction mixture was allowed to warm to rt. After 2 h, the reaction was placed in a freezer overnight. After stirring for an additional 1 h at rt, the reaction was quenched through the addition of sat. NaHCO₃(1 mL) and was extracted with EtOAc (1 mL×3). The combined extracts were dried over Na2SO4, were filtered, and were concentrated. The resulting crude material was dissolved in MeOH (1 mL), was heated at 40° C. overnight, and was concentrated. The residue was purified by HPLC (Atlantis T3 column, 5-50% MeCN-water-0.1% HCO2H) to give 12.9 mg of the title compound as a formate salt. MS (ESI+) m/z: 655.39 [M+H]+; 1H NMR (400 MHz, Methanol-d) δ 8.35 (s, 3H), 5.43 (dq, 1H), 4.49 (d, 1H), 4.10 (d, 1H), 3.88 (ddt, 1H), 3.73 (ddd, 1H), 3.65-3.36 (m, 7H), 3.18 (d, 3H), 3.07 (t, 1H), 3.02 (d, 3H), 2.98-2.85 (m, 2H), 2.82 (d, 6H), 2.68-2.53 (m, 4H), 2.53-2.44 (m, 1H), 2.26 (d, 1H), 2.09 (s, 3H), 2.07-2.01 (m, 1H), 1.83 (d, 1H), 1.59-1.43 (m, 4H), 1.42-1.37 (m, 5H), 1.37-1.33 (m, 9H), 1.33-1.27 (m, 3H), 1.07 (d, 3H).

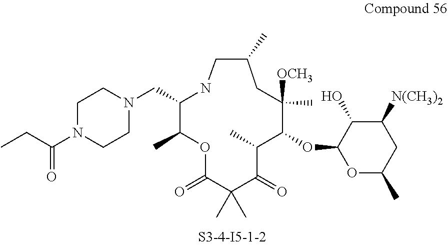 [SMILES: CCC(=O)N1CCN(C[C@@H]2NC[C@H](C)C[C@@](C)(OC)[C@H](O[C@@H]3O[C@H](C)C[C@H](N(C)C)[C@H]3O)[C@@H](C)C(=O)C(C)(C)C(=O)O[C@H]2C)CC1]

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-3-((4-propionylpiperazin-1-yl)methyl)-1-oxa-4-azacyclotridecane-11,13-dione (S3-4-I5-1-2)

Prepared according to the methods of S3-4-I5-1-1 from S3-1-I5-1 and propionyl chloride to provide the title compound as a formate salt. MS (ESI+) m/z: 669.43 [M+H]+; 1H NMR (400 MHz, Methanol-d) δ 8.43 (s, 3H), 5.44 (dt, 1H), 4.49 (d, 1H), 4.10 (d, 1H), 3.88 (ddd, 1H), 3.73 (ddd, 1H), 3.65-3.37 (m, 7H), 3.18 (s, 3H), 3.10-2.98 (m, 4H), 2.98-2.84 (m, 2H), 2.82 (s, 6H), 2.59 (dt, 3H), 2.53-2.45 (m, 1H), 2.40 (q, 2H), 2.27 (d, 1H), 2.03 (ddd, 1H), 1.83 (d, 1H), 1.61-1.52 (m, 1H), 1.50 (s, 3H), 1.43-1.33 (m, 14H), 1.33-1.27 (m, 3H), 1.14-1.03 (m, 6H).

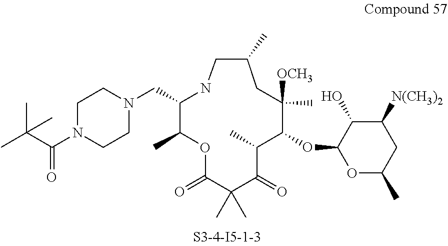 [SMILES: CO[C@]1(C)C[C@@H](C)CN[C@@H](CN2CCN(C(=O)C(C)(C)C)CC2)[C@H](C)OC(=O)C(C)(C)C(=O)[C@H](C)[C@H]1O[C@@H]1O[C@H](C)C[C@H](N(C)C)[C@H]1O]

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-3-((4-pivaloylpiperazin-1-yl)methyl)-1-oxa-4-azacyclotridecane-11,13-dione (S3-4-I5-1-3)

Prepared according to the methods of S3-4-15-1-1 from S3-1-15-1 and pivaloyl chloride to provide the title compound as a formate salt. MS (ESI+) m/z: 697.33 [M+H]+; 1H NMR (400 MHz, Methanol-d) δ 8.37 (s, 3H), 5.42 (dq, 1H), 4.49 (d, 1H), 4.10 (d, 1H), 3.93-3.81 (m, 1H), 3.78-3.59 (m, 5H), 3.54-3.46 (m, 1H), 3.46-3.36 (m, 2H), 3.18 (d, 3H), 3.07 (t, 1H), 3.02 (s, 3H), 2.97-2.83 (m, 2H), 2.81 (d, 6H), 2.65-2.55 (m, 3H), 2.55-2.47 (m, 2H), 2.33-2.20 (m, 1H), 2.02 (ddd, 1H), 1.82 (d, 1H), 1.59-1.44 (m, 4H), 1.42-1.33 (m, 13H), 1.31 (d, 3H), 1.26 (d, 9H), 1.10-1.03 (m, 3H).

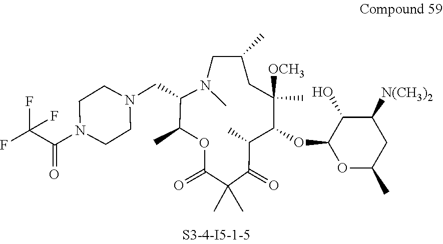 [SMILES: CO[C@]1(C)C[C@@H](C)CN(C)[C@@H](CN2CCN(C(=O)C(F)(F)F)CC2)[C@H](C)OC(=O)C(C)(C)C(=O)[C@H](C)[C@H]1O[C@@H]1O[C@H](C)C[C@H](N(C)C)[C@H]1O]

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-3-((4-(2,2,2-trifluoroacetyl)piperazin-1-yl)methyl)-1-oxa-4-azacyclotridecane-11,13-dione (S3-4-I5-1-5)

Prepared according to the methods of S3-4-15-1-1 from S3-1-15-1 and trifluoroacetic anhydride to provide the title compound as a formate salt. MS (ESI+) m/z: 709.27 [M+H]+; 1H NMR (400 MHz, Methanol-d) δ 8.49 (s, 3H), 5.46 (s, 1H), 4.51 (d, 1H), 4.12 (d, 1H), 3.89 (s, 1H), 3.80-3.65 (m, 5H), 3.53 (dd, 1H), 3.49-3.34 (m, 2H), 3.19 (s, 3H), 3.04 (s, 4H), 2.93 (dd, 2H), 2.80 (s, 6H), 2.74-2.58 (m, 5H), 2.28 (s, 1H), 2.06-1.99 (m, 1H), 1.85 (d, 1H), 1.52 (s, 4H), 1.45-1.35 (m, 13H), 1.33 (d, 3H), 1.09 (d, 3H).

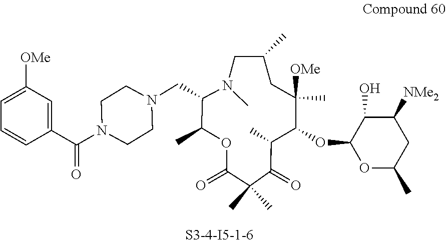

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-3-((4-(3-methoxybenzoyl)piperazin-1-yl)methyl)-2,4,6,8,10,12,12-heptamethyl-1-oxa-4-azacyclotridecane-11,13-dione (S3-4-I5-1-6)

Prepared according to the methods of S3-4-I5-1-1 from S3-1-I5-1 and 3-methoxybenzoyl chloride to provide the title compound as a free base. MS (ESI+) m/z: 747.3 [M+H]+; ¹H NMR (400 MHz, METHANOL-d4) δ=8.57 (br s, 2H), 7.40 (br t, 1H), 7.07 (br d, 1H), 7.02-6.95 (m, 2H), 5.47 (br s, 1H), 4.50 (br d, 1H), 4.13 (br d, 1H), 3.91 (br s, 1H), 3.87-3.83 (m, 3H), 3.83-3.63 (m, 3H), 3.61-3.48 (m, 3H), 3.48-3.38 (m, 2H), 3.25-3.19 (m, 3H), 3.04 (br s, 4H), 3.01-2.90 (m, 2H), 2.71 (br s, 7H), 2.68-2.45 (m, 5H), 2.43-2.21 (m, 1H), 1.99 (br d, 1H), 1.87 (br d, 1H), 1.56-1.48 (m, 4H), 1.44-1.36 (m, 12H), 1.35-1.30 (m, 3H), 1.14-1.06 (m, 3H).

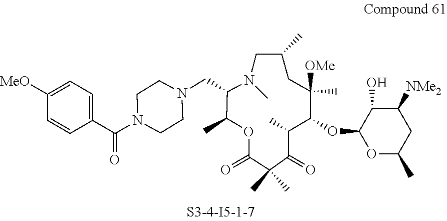

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-3-((4-(4-methoxybenzoyl)piperazin-1-yl)methyl)-2,4,6,8,10,12,12-heptamethyl-1-oxa-4-azacyclotridecane-11,13-dione (S3-4-I5-1-7)

Prepared according to the methods of S3-4-I5-1-1 from S3-1-I5-1 and 4-methoxybenzoyl chloride to provide the title compound as a formate salt. MS (ESI+) m/z: 747.5 [M+H]+; ¹H NMR (400 MHz, METHANOL-d4) δ=8.57 (br s, 2H), 7.40 (br t, 1H), 7.07 (br d, 1H), 7.02-6.95 (m, 2H), 5.47 (br s, 1H), 4.50 (br d, 1H), 4.13 (br d, 1H), 3.91 (br s, 1H), 3.87-3.83 (m, 3H), 3.83-3.63 (m, 3H), 3.61-3.48 (m, 3H), 3.48-3.38 (m, 2H), 3.25-3.19 (m, 3H), 3.04 (br s, 4H), 3.01-2.90 (m, 2H), 2.71 (br s, 7H), 2.68-2.45 (m, 5H), 2.43-2.21 (m, 1H), 1.99 (br d, 1H), 1.87 (br d, 1H), 1.56-1.48 (m, 4H), 1.44-1.36 (m, 12H), 1.35-1.30 (m, 3H), 1.14-1.06 (m, 3H).

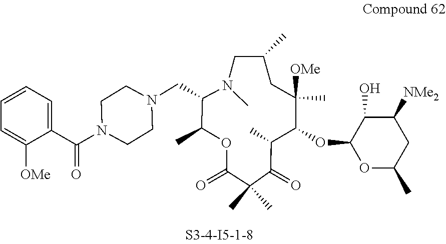

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-3-((4-(2-methoxybenzoyl)piperazin-1-yl)methyl)-2,4,6,8,10,12,12-heptamethyl-1-oxa-4-azacyclotridecane-11,13-dione (S3-4-I5-1-8)

Prepared according to the methods of S3-4-I5-1-1 from S3-1-I5-1 and 2-methoxybenzoyl chloride to provide the title compound as a formate salt. MS (ESI+) m/z: 747.5 [M+H]+; 1H NMR (400 MHz, METHANOL-d4) δ=8.53 (br s, 1H), 7.46-7.39 (m, 1H), 7.21 (br d, 1H), 7.11-6.99 (m, 2H), 5.48-5.39 (m, 1H), 4.51-4.44 (m, 1H), 4.11 (br d, 1H), 3.91-3.77 (m, 5H), 3.77-3.62 (m, 2H), 3.60-3.45 (m, 1H), 3.44-3.34 (m, 2H), 3.19 (br s, 4H), 3.12-2.50 (m, 15H), 2.48-2.37 (m, 1H), 2.36-2.15 (m, 1H), 1.96 (br d, 1H), 1.90-1.77 (m, 1H), 1.55-1.15 (m, 18H), 1.12-1.03 (m, 2H).

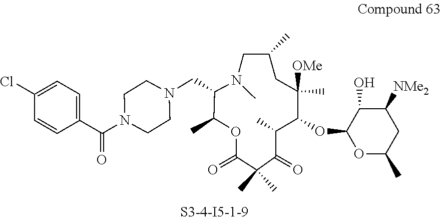

(2S,3S,6R,8R,9R,10R)-3-((4-(4-chlorobenzoyl)piperazin-1-yl)methyl)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-1-oxa-4-azacyclotridecane-11,13-dione (S3-4-I5-1-9)

Prepared according to the methods of S3-4-I5-1-1 from S3-1-I5-1 and 4-chlorobenzoyl chloride to provide the title compound as a formate salt. MS (ESI+) m/z: 751.2 [M+H]+; ¹H NMR (400 MHz, METHANOL-d4) δ=7.51-7.46 (m, 2H), 7.44-7.39 (m, 2H), 4.59-4.50 (m, 1H), 4.40 (d, 1H), 4.14-4.03 (m, 1H), 3.94-3.64 (m, 3H), 3.63-3.41 (m, 5H), 3.29-3.17 (m, 2H), 3.05-2.99 (m, 1H), 2.93 (s, 3H), 2.90-2.81 (m, 1H), 2.80-2.32 (m, 18H), 2.30-2.12 (m, 2H), 1.80-1.65 (m, 3H), 1.51 (s, 1H), 1.43-1.21 (m, 20H), 1.20-1.07 (m, 2H), 0.90 (d, 2H).

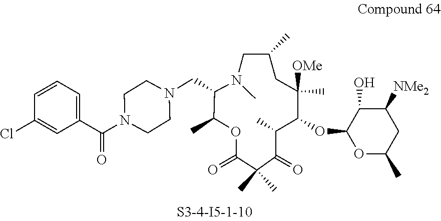

(2S,3S,6R,8R,9R,10R)-3-((4-(3-chlorobenzoyl)piperazin-1-yl)methyl)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-1-oxa-4-azacyclotridecane-11,13-dione (S3-4-I5-1-10)

Prepared according to the methods of S3-4-I5-1-1 from S3-1-I5-1 and 3-chlorobenzoyl chloride to provide the title compound as a formate salt. MS (ESI+) m/z: 751.4 [M+H]+; ¹H NMR (400 MHz, METHANOL-d4) δ=8.58-8.53 (m, 1H), 7.54-7.42 (m, 3H), 7.34 (br d, 1H), 5.52-5.40 (m, 1H), 4.41 (d, 1H), 4.15-3.98 (m, 1H), 3.95-3.65 (m, 2H), 3.63-3.34 (m, 4H), 3.29-3.14 (m, 2H), 3.11-2.99 (m, 1H), 2.99-2.83 (m, 3H), 2.76-2.59 (m, 3H), 2.59-2.30 (m, 12H), 2.28-2.09 (m, 1H), 1.95-1.63 (m, 3H), 1.51 (s, 1H), 1.44-1.14 (m, 17H), 1.13-1.08 (m, 1H), 0.91 (br d, 2H).

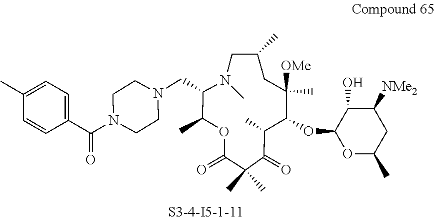

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-3-((4-(4-methylbenzoyl)piperazin-1-yl)methyl)-1-oxa-4-azacyclotridecane-11,13-dione (S3-4-I5-1-11)

Prepared according to the methods of S3-4-15-1-1 from S3-1-15-1 and 4-methylbenzoyl chloride to provide the title compound as a formate salt. MS (ESI+) m/z: 731.3 [M+H]+; ¹H NMR (400 MHz, METHANOL-d4) δ=7.35-7.27 (m, 4H), 5.47 (br dd, 0.4H), 4.72-4.52 (m, 1H), 4.42 (d, 1H), 4.17-4.05 (m, 1H), 3.95-3.73 (m, 2H), 3.64-3.46 (m, 5H), 3.31-3.26 (m, 1H), 3.25-3.19 (m, 1H), 2.95 (s, 3H), 2.91-2.83 (m, 1H), 2.70-2.50 (m, 6H), 2.42-2.34 (m, 14H), 2.27-2.14 (m, 1H), 1.82-1.68 (m, 3H), 1.45-1.25 (m, 21H), 1.22-1.07 (m, 2H), 0.92 (d, 3H).

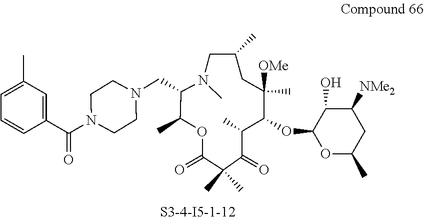

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-3-((4-(3-methylbenzoyl)piperazin-1-yl)methyl)-1-oxa-4-azacyclotridecane-11,13-dione (S3-4-I5-1-12)

Prepared according to the methods of S3-4-15-1-1 from S3-1-15-1 and 3-methylbenzoyl chloride to provide the title compound as a formate salt. MS (ESI+) m/z: 731.5 [M+H]+; ¹H NMR (400 MHz, METHANOL-d4) δ=8.53 (s, 1H), 7.42-7.27 (m, 2H), 7.26-7.17 (m, 2H), 5.51-5.39 (m, 1H), 4.54-4.45 (m, 1H), 4.12 (br d, 1H), 3.94-3.84 (m, 1H), 3.84-3.63 (m, 3H), 3.60-3.34 (m, 5H), 3.28-3.17 (m, 4H), 3.17-2.88 (m, 7H), 2.77-2.59 (m, 9H), 2.57-2.45 (m, 2H), 2.39 (s, 3H), 2.35-2.13 (m, 1H), 2.01-1.91 (m, 1H), 1.85 (br d, 1H), 1.56-1.44 (m, 4H), 1.44-1.25 (m, 16H), 1.09 (br d, 3H).

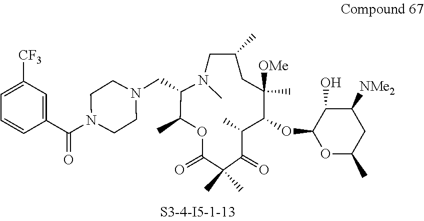

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-3-((4-(3-(trifluoromethyl)benzoyl)piperazin-1-yl)methyl)-1-oxa-4-azacyclotridecane-11,13-dione (S3-4-I51-13)

Prepared according to the methods of S3-4-I5-1-1 from S3-1-I5-1 and 3-trifluoromethylbenzoyl chloride to provide the title compound as a formate salt. MS (ESI+) m/z: 785.5 [M+H]+; ¹H NMR (400 MHz, METHANOL-d4) δ=7.83-7.77 (m, 1H), 7.75-7.66 (m, 3H), 5.52-5.39 (m, 1H), 4.41 (d, 1H), 4.17-4.01 (m, 1H), 3.78 (br s, 2H), 3.65-3.39 (m, 4H), 3.29-3.23 (m, 1H), 3.19 (s, 1H), 3.02 (s, 1H), 2.93 (s, 3H), 2.85 (br d, 1H), 2.70-2.43 (m, 6H), 2.43-2.25 (m, 10H), 2.19 (br d, 1H), 1.91-1.62 (m, 3H), 1.51 (s, 1H), 1.44-1.22 (m, 18H), 1.20-1.04 (m, 2H), 0.95-0.86 (m, 2H).

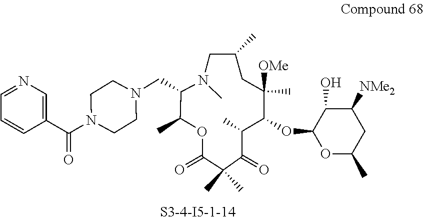

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-3-((4-nicotinoylpiperazin-1-yl)methyl)-1-oxa-4-azacyclotridecane-11,13-dione (S3-4-I5-1-14)

Prepared according to the methods of S3-4-I5-1-1 from S3-1-I5-1 and nicotinoyl chloride to provide the title compound as a formate salt. MS (ESI+) m/z: 718.5 [M+H]+; ¹H NMR (400 MHz, METHANOL-d4) δ=8.69-8.51 (m, 3H), 7.90 (br d, 1H), 7.58-7.49 (m, 1H), 5.45 (br dd, 0.4H), 4.66-4.50 (m, 1H), 4.47-4.34 (m, 1H), 4.17-4.01 (m, 1H), 3.97-3.67 (m, 3H), 3.64-3.38 (m, 4H), 3.27-3.14 (m, 2H), 3.12-3.01 (m, 2H), 2.98-2.81 (m, 3H), 2.79-2.14 (m, 17H), 1.93-1.59 (m, 3H), 1.54-1.19 (m, 20H), 1.17-1.01 (m, 2H), 0.90 (br d, 1H).

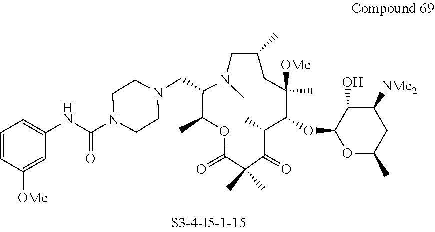

4-(((2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-11,13-dioxo-1-oxa-4-azacyclotridecan-3-yl)methyl)-N-(3-methoxyphenyl)piperazine-1-carboxamide (S3-4-I5-1-15)

Prepared according to the methods of S3-4-I5-1-1 from S3-1-I5-1 and 3-methoxyphenyl isocyante to provide the title compound as a formate salt. MS (ESI+) m/z: 762.5 [M+H]+; ¹H NMR (400 MHz, METHANOL-d4) δ=8.55 (br s, 1H), 7.18-7.11 (m, 1H), 7.04 (s, 1H), 6.91 (br d, 1H), 6.63-6.56 (m, 1H), 5.45 (br dd, 1H), 4.67-4.50 (m, 1H), 4.41 (br d, 1H), 4.10 (br t, 1H), 3.96-3.83 (m, 1H), 3.77 (s, 3H), 3.57 (br s, 3H), 3.52 (br s, 3H), 3.27-3.17 (m, 3H), 3.14-2.99 (m, 3H), 2.99-2.83 (m, 3H), 2.81-2.72 (m, 1H), 2.66 (br s, 2H), 2.62-2.46 (m, 4H), 2.41 (br s, 4H), 2.38-2.19 (m, 6H), 1.96-1.65 (m, 3H), 1.51 (br s, 2H), 1.46-1.21 (m, 19H), 1.14-1.07 (m, 2H), 0.99-0.84 (m, 1H).

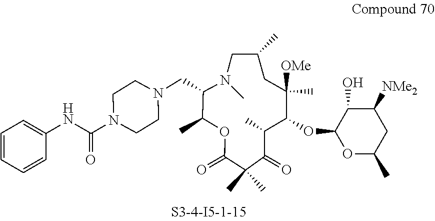

4-(((2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-11,13-dioxo-1-oxa-4-azacyclotridecan-3-yl)methyl)-N-phenylpiperazine-1-carboxamide (S3-4-I5-1-16)

Prepared according to the methods of S3-4-15-1-1 from S3-1-15-1 and phenyl isocyante to provide the title compound as a formate salt. MS (ESI+) m/z: 732.5 [M+H]+; 1H NMR (400 MHz, METHANOL-d4) δ=8.63-8.46 (m, 1H), 7.36-7.31 (m, 2H), 7.30-7.21 (m, 2H), 7.02 (q, 1H), 5.49-5.41 (m, 1H), 4.85-4.82 (m, 1H), 4.65-4.53 (m, 1H), 4.41 (br dd, 1H), 4.25-3.99 (m, 1H), 3.90 (br s, 1H), 3.69-3.42 (m, 7H), 3.28-3.17 (m, 3H), 3.14-3.00 (m, 2H), 3.00-2.85 (m, 3H), 2.69-2.46 (m, 6H), 2.43-2.28 (m, 8H), 2.25-2.09 (m, 1H), 1.95-1.82 (m, 1H), 1.81-1.62 (m, 2H), 1.51 (s, 1H), 1.45-1.20 (m, 18H), 1.10 (br d, 1H), 0.92 (br d, 1H).

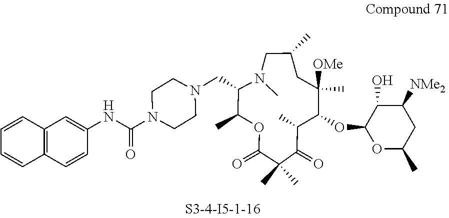

4-(((2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-11,13-dioxo-1-oxa-4-azacyclotridecan-3-yl)methyl)-N-(naphthalen-2-yl)piperazine-1-carboxamide (S3-4-I5-1-16)

Prepared according to the methods of S3-4-15-1-1 from S3-1-15-1 and 2-naphthyl isocyante to provide the title compound as a formate salt. MS (ESI+) m/z: 782.5 [M+H]+; ¹H NMR (400 MHz, METHANOL-d4) δ=8.57 (br s, 1H), 7.86 (d, 1H), 7.76 (dd, J 3H), 7.50 (dd, 1H), 7.46-7.40 (m, 1H), 7.39-7.33 (m, 1H), 5.53-5.41 (m, 1H), 4.48-4.37 (m, 1H), 4.17-4.03 (m, 1H), 3.93 (br d, 1H), 3.71-3.45 (m, 6H), 3.23 (s, 2H), 3.11-3.01 (m, 2H), 3.01-2.83 (m, 3H), 2.81-2.50 (m, 7H), 2.46-2.26 (m, 10H), 1.97-1.69 (m, 3H), 1.56-1.50 (m, 2H), 1.47-1.35 (m, 10H), 1.31-1.22 (m, 10H), 1.17-1.08 (m, 2H), 0.93 (br d, 1H).

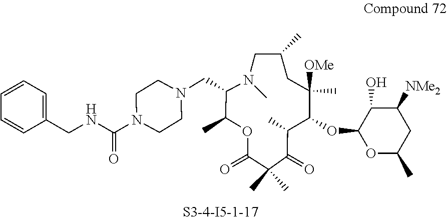

N-benzyl-4-(((2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-11,13-dioxo-1-oxa-4-azacyclotridecan-3-yl)methyl)piperazine-1-carboxamide (S3-4-I5-1-17)

Prepared according to the methods of S3-4-I5-1-1 from S3-1-I5-1 and benzyl isocyante to provide the title compound as a formate salt. MS (ESI+) m/z: 746.5 [M+H]+; ¹HNMR (400 MHz, METHANOL-d4) δ=8.63-8.52 (m, 0.3H), 7.34-7.22 (m, 5H), 5.46 (br dd, 1H), 4.61 (br s, 1H), 4.47-4.31 (m, 3H), 4.22-4.03 (m, 1H), 3.98-3.84 (m, 1H), 3.67-3.46 (m, 5H), 3.46-3.38 (m, 2H), 3.29-3.18 (m, 3H), 3.12-3.00 (m, 3H), 3.00-2.84 (m, 3H), 2.82-2.59 (m, 3H), 2.57-2.39 (m, 7H), 2.39-2.11 (m, 6H), 1.97-1.63 (m, 3H), 1.53 (s, 2H), 1.46-1.21 (m, 20H), 1.11 (br d, 2H), 1.01-0.84 (m, 1H).

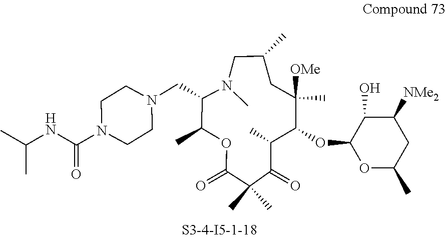

4-(((2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,4,6,8,10,12,12-heptamethyl-11,13-dioxo-1-oxa-4-azacyclotridecan-3-yl)methyl)-N-isopropylpiperazine-1-carboxamide (S3-4-I5-1-18)

Prepared according to the methods of S3-4-I5-1-1 from S3-1-I5-1 and isopropyl isocyante to provide the title compound as a formate salt. MS (ESI+) m/z: 698.5 [M+H]+; ¹H NMR (400 MHz, METHANOL-d4) δ=8.57-8.52 (m, 0.4H), 5.51-5.37 (m, 1H), 4.41 (br d, 1H), 4.13-4.05 (m, 1H), 3.93-3.83 (m, 2H), 3.63-3.45 (m, 3H), 3.44-3.33 (m, 5H), 3.29-3.13 (m, 3H), 3.02 (s, 2H), 2.94 (s, 2H), 2.86 (br d, 1H), 2.71-2.55 (m, 3H), 2.53-2.27 (m, 15H), 2.22-2.11 (m, 1H), 1.89-1.65 (m, 3H), 1.51 (s, 1H), 1.44-1.19 (m, 21H), 1.16-1.07 (m, 9H), 0.95-0.86 (m, 2H).

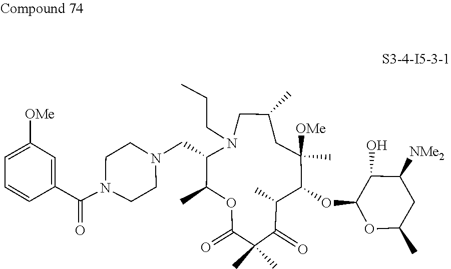

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-3-((4-(3-methoxybenzoyl)piperazin-1-yl)methyl)-2,6,8,10,12,12-hexamethyl-4-propyl-1-oxa-4-azacyclotridecane-11,13-dione (S3-4-I5-3-1)

Prepared according to the methods of S3-4-I5-1-1 from S3-1-I5-3 and 3-methoxybenzoyl chloride to provide the title compound as a formate salt. MS (ESI+) m/z: 775.5 [M+H]+; 1H NMR (400 MHz, METHANOL-d4) δ=7.39 (t, 1H), 7.05 (dd, 1H), 6.99-6.94 (m, 2H), 4.42 (br d, 1H), 4.10 (br d, 1H), 3.84 (s, 3H), 3.76 (br s, 2H), 3.69-3.48 (m, 4H), 3.48-3.37 (m, 3H), 3.30-3.13 (m, 1H), 2.95-2.72 (m, 5H), 2.70-2.51 (m, 4H), 2.53-2.33 (m, 13H), 2.18 (s, 2H), 1.90-1.64 (m, 3H), 1.52-1.36 (m, 9H), 1.34-1.18 (m, 16H), 0.97-0.84 (m, 6H).

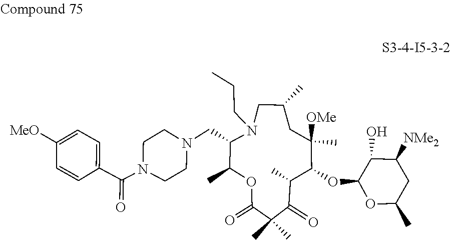

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-3-((4-(4-methoxybenzoyl)piperazin-1-yl)methyl)-2,6,8,10,12,12-hexamethyl-4-propyl-1-oxa-4-azacyclotridecane-11,13-dione (S3-4-I5-3-2)

Prepared according to the methods of S3-4-I5-1-1 from S3-1-I5-3 and 4-methoxybenzoyl chloride to provide the title compound as a formate salt. MS (ESI+) m/z: 775.5 [M+H]+; ¹H NMR (400 MHz, METHANOL-d4) δ=7.42-7.36 (m, 2H), 7.03-6.97 (m, 2H), 5.32 (br dd, 1H), 4.63-4.44 (m, 2H), 4.17-3.92 (m, 3H), 3.86-3.82 (m, 4H), 3.78-3.50 (m, 7H), 3.49-3.35 (m, 4H), 3.26-3.11 (m, 2H), 3.08-2.88 (m, 6H), 2.81 (br s, 2H), 2.76 (br s, 4H), 2.73-2.59 (m, 3H), 2.59-2.50 (m, 2H), 2.49-2.34 (m, 2H), 2.34-2.23 (m, 1H), 2.12 (td, 2H), 2.00 (br d, 1H), 1.83 (br d, 1H), 1.53-1.25 (m, 22H), 1.16-1.04 (m, 5H), 0.94-0.83 (m, 1H).

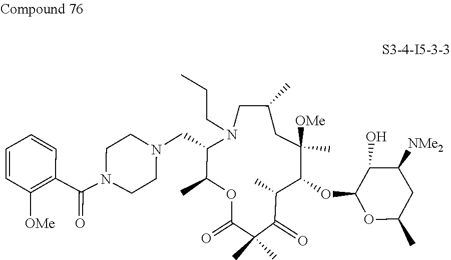

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-3-((4-(2-methoxybenzoyl)piperazin-1-yl)methyl)-2,6,8,10,12,12-hexamethyl-4-propyl-1-oxa-4-azacyclotridecane-11,13-dione (S3-4-I5-3-3)

Prepared according to the methods of S3-4-I5-1-1 from S3-1-I5-3 and 2-methoxybenzoyl chloride to provide the title compound as a formate salt. MS (ESI+) m/z: 775.5 [M+H]+; ¹H NMR (400 MHz, METHANOL-d4) δ=7.47-7.39 (m, 1H), 7.25-7.15 (m, 1H), 7.13-6.96 (m, 2H), 4.46-4.37 (m, 1H), 4.08 (d, 1H), 3.89-3.66 (m, 5H), 3.64-3.46 (m, 2H), 3.25-3.16 (m, 3H), 2.97-2.90 (m, 3H), 2.86-2.70 (m, 2H), 2.67-2.35 (m, 12H), 2.32-2.09 (m, 5H), 1.84-1.72 (m, 1H), 1.68-1.54 (m, 1H), 1.51-1.19 (m, 22H), 1.10 (br d, 3H), 0.96-0.78 (m, 6H).

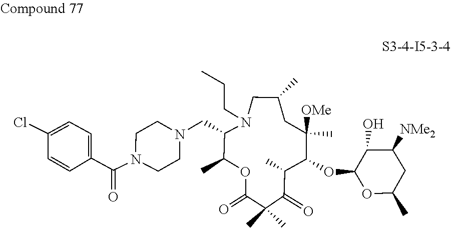

(2S,3S,6R,8R,9R,10R)-3-((4-(4-chlorobenzoyl)piperazin-1-yl)methyl)-9-(((2S,3R,4S,6R)-4-(dimethylamino)-3-hydroxy-6-methyltetrahydro-2H-pyran-2-yl)oxy)-8-methoxy-2,6,8,10,12,12-hexamethyl-4-propyl-1-oxa-4-azacyclotridecane-11,13-dione (S3-4-I5-3-4)

Prepared according to the methods of S3-4-I5-1-1 from S3-1-I5-3 and 4-chlorobenzoyl chloride to provide the title compound as a formate salt. MS (ESI+) m/z: 779.5 [M+H]+; ¹H NMR (400 MHz, METHANOL-d4) δ=7.50-7.38 (m, 4H), 4.63-4.51 (m, 1H), 4.40 (br d, 1H), 4.14-3.99 (m, 1H), 3.78-3.65 (m, 2H), 3.63-3.45 (m, 3H), 3.45-3.35 (m, 2H), 3.27-3.04 (m, 1H), 3.02-2.85 (m, 4H), 2.85-2.63 (m, 3H), 2.60-2.35 (m, 13H), 2.34-2.05 (m, 4H), 1.85-1.68 (m, 2H), 1.66-1.56 (m, 1H), 1.49-1.41 (m, 5H), 1.41-0.97 (m, 20H), 0.93-0.82 (m, 6H).

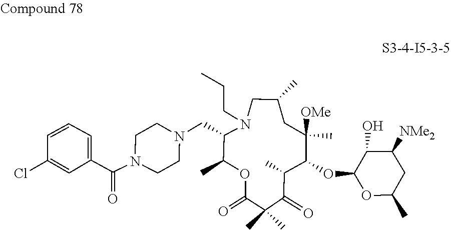

(2S,3S,6R,8R,9R,10R)-3-((4-(3-chlorobenzoyl)piperazin-1-yl)methyl)-9-(((2S,3R,4S,6R)-4-(dimethylamino)-3-hydroxy-6-methyltetrahydro-2H-pyran-2-yl)oxy)-8-methoxy-2,6,8,10,12,12-hexamethyl-4-propyl-1-oxa-4-azacyclotridecane-11,13-dione (S3-4-I5-3-5)

Prepared according to the methods of S3-4-I5-1-1 from S3-1-I5-3 and 3-chlorobenzoyl chloride to provide the title compound as a formate salt. MS (ESI+) m/z: 779.5 [M+H]+; ¹H NMR (400 MHz, METHANOL-d4) δ=7.55-7.39 (m, 3H), 7.33 (d, 1H), 4.40 (br d, 1H), 4.07 (d, 1H), 3.73 (br d, 2H), 3.61-3.47 (m, 3H), 3.40 (br s, 2H), 3.25-3.05 (m, 1H), 2.99-2.89 (m, 3H), 2.85-2.78 (m, 1H), 2.74-2.56 (m, 1H), 2.54-2.25 (m, 17H), 2.23-2.00 (m, 2H), 1.81-1.70 (m, 1H), 1.68-1.52 (m, 1H), 1.50-1.31 (m, 10H), 1.29-0.97 (m, 19H), 0.95-0.70 (m, 7H).

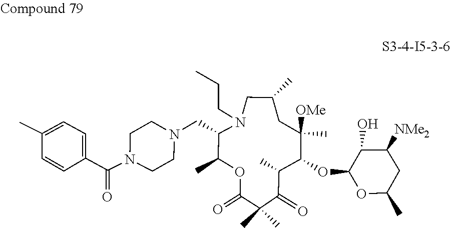

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,6,8,10,12,12-hexamethyl-3-((4-(4-methylbenzoyl)piperazin-1-yl)methyl)-4-propyl-1-oxa-4-azacyclotridecane-11,13-dione (S3-4-I5-3-6)

Prepared according to the methods of S3-4-I5-1-1 from S3-1-I5-3 and 4-methylbenzoyl chloride to provide the title compound as a formate salt. MS (ESI+) m/z: 759.5 [M+H]+; ¹H NMR (400 MHz, METHANOL-d4) δ=7.34-7.27 (m, 4H), 4.42 (br d, 1H), 4.10 (d, 1H), 3.75 (br s, 2H), 3.65-3.39 (m, 5H), 3.31-3.21 (m, 1H), 2.95 (s, 3H), 2.85 (br d, 1H), 2.76-2.52 (m, 4H), 2.49-2.32 (m, 16H), 2.28-2.12 (m, 2H), 1.90-1.56 (m, 3H), 1.52-1.36 (m, 9H), 1.34-1.22 (m, 14H), 1.22-1.16 (m, 1H), 1.13 (br s, 1H), 0.97-0.84 (m, 7H).

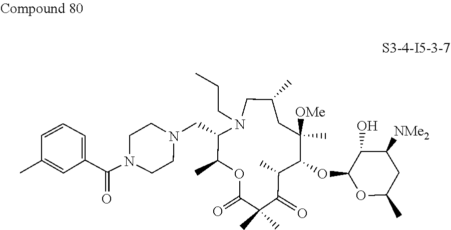

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,6,8,10,12,12-hexamethyl-3-((4-(3-methylbenzoyl)piperazin-1-yl)methyl)-4-propyl-1-oxa-4-azacyclotridecane-11,13-dione (S3-4-I5-3-7)

Prepared according to the methods of S3-4-15-1-1 from S3-1-I5-3 and 3-methylbenzoyl chloride to provide the title compound as a formate salt. MS (ESI+) m/z: 759.5 [M+H]+; ¹H NMR (400 MHz, METHANOL-d4) δ=7.32 (td, 2H), 7.24-7.11 (m, 2H), 4.41 (br d, 1H), 4.08 (br d, 1H), 3.81-3.66 (m, 2H), 3.61-3.36 (m, 4H), 3.25-3.13 (m, 1H), 2.93 (s, 3H), 2.87-2.78 (m, 1H), 2.74-2.54 (m, 2H), 2.53-2.26 (m, 19H), 2.24-2.03 (m, 2H), 1.79-1.72 (m, 1H), 1.70-1.55 (m, 1H), 1.50-1.40 (m, 6H), 1.36 (s, 4H), 1.29-0.99 (m, 19H), 0.93-0.74 (m, 8H).

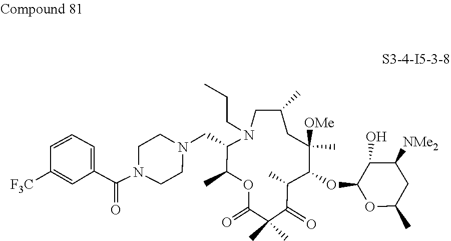

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,6,8,10,12,12-hexamethyl-4-propyl-3-((4-(3-(trifluoromethyl)benzoyl)piperazin-1-yl)methyl)-1-oxa-4-azacyclotridecane-11,13-dione (S3-4-I5-3-8)

Prepared according to the methods of S3-4-15-1-1 from S3-1-I5-3 and 3-trifluoromethylbenzoyl chloride to provide the title compound as a formate salt. MS (ESI+) m/z: 813.5 [M+H]+; ¹H NMR (400 MHz, METHANOL-d4) δ=7.82-7.77 (m, 1H), 7.74-7.65 (m, 3H), 4.40 (br d, 1H), 4.08 (d, 1H), 3.77 (br s, 2H), 3.68-3.45 (m, 3H), 3.41 (br s, 2H), 3.29-3.19 (m, 1H), 2.93 (s, 3H), 2.83 (br d, 1H), 2.73-2.56 (m, 3H), 2.52-2.32 (m, 13H), 2.25-2.06 (m, 2H), 1.88-1.53 (m, 3H), 1.50-1.34 (m, 9H), 1.32-1.18 (m, 14H), 0.94-0.82 (m, 6H).

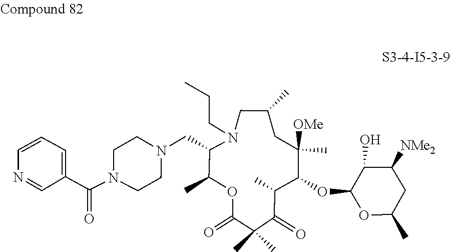

(2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,6,8,10,12,12-hexamethyl-3-((4-nicotinoylpiperazin-1-yl)methyl)-4-propyl-1-oxa-4-azacyclotridecane-11,13-dione (S3-4-I5-3-9)

Prepared according to the methods of S3-4-I5-1-1 from S3-1-I5-3 and nicotinoyl chloride to provide the title compound as a formate salt. MS (ESI+) m/z: 746.5 [M+H]+; ¹H NMR (400 MHz, METHANOL-d4) δ=8.64 (dd, 2H), 8.61 (d, 1H), 7.90 (td, 1H), 7.53 (dd, 1H), 5.02-4.89 (m, 1H), 4.41 (br d, 1H), 4.08 (d, 1H), 3.77 (br s, 2H), 3.64-3.35 (m, 5H), 3.28-3.13 (m, 1H), 2.94 (s, 3H), 2.83 (br d, 1H), 2.78-2.58 (m, 3H), 2.54-2.32 (m, 13H), 2.29-2.09 (m, 3H), 1.92-1.69 (m, 2H), 1.68-1.55 (m, 1H), 1.50-1.35 (m, 9H), 1.33-1.03 (m, 16H), 0.95-0.82 (m, 6H).

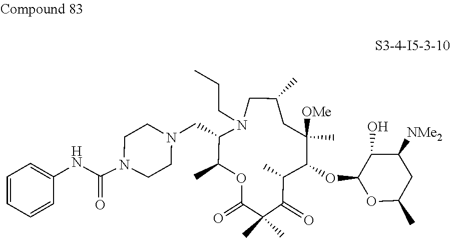

4-(((2S,3S,6R,8R,9R,10R)-9-(((2S,3R,4S,6R)-4-(dimethylamino)-3-hydroxy-6-methyltetrahydro-2H-pyran-2-yl)oxy)-8-methoxy-2,6,8,10,12,12-hexamethyl-11,13-dioxo-4-propyl-1-oxa-4-azacyclotridecan-3-yl)methyl)-N-phenylpiperazine-1-carboxamide (S3-4-I5-3-10)

Prepared according to the methods of S3-4-15-1-1 from S3-1-15-3 and phenyl isocyanate to provide the title compound as a formate salt. MS (ESI+) m/z: 760.5 [M+H]+; ¹H NMR (400 MHz, METHANOL-d4) δ=7.34 (br d, 2H), 7.25 (br t, 2H), 7.05-6.98 (m, 1H), 4.41 (br d, 1H), 4.13-4.07 (m, 1H), 3.67-3.46 (m, 8H), 3.02-2.91 (m, 4H), 2.90-2.81 (m, 1H), 2.77-2.64 (m, 2H), 2.62-2.51 (m, 3H), 2.49-2.35 (m, 12H), 2.26-2.11 (m, 2H), 1.77 (br d, 1H), 1.73-1.55 (m, 2H), 1.52-1.42 (m, 6H), 1.42-1.35 (m, 4H), 1.32-1.24 (m, 12H), 1.16-1.05 (m, 1H), 0.97-0.84 (m, 6H).

The following Examples were prepared according to the methods of Scheme 3, substituting the appropriate Intermediate from Table 1.

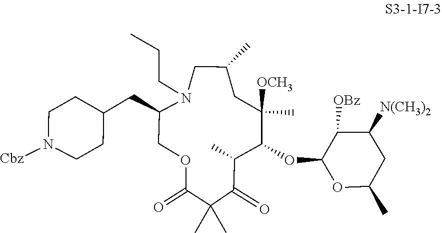

Benzyl 4-(((3R,6R,8R,9R,10R)-9-(((2S,3R,4S,6R)-3-(benzoyloxy)-4-(dimethylamino)-6-methyltetrahydro-2H-pyran-2-yl)oxy)-8-methoxy-6,8,10,12,12-pentamethyl-11,13-dioxo-4-propyl-1-oxa-4-azacyclotridecan-3-yl)methyl)piperidine-1-carboxylate (S3-1-I7-3)

An oven-dried flask was evacuated and back-filled with nitrogen (2 times) before cooling to rt. S2-1-I7-3 (196 mg, 0.226 mmol, prepared as described in Scheme 1 from 17 and propionaldehyde) in ethanol (4 mL) was added to the flask, which was then evacuated and back-filled with nitrogen (2 times). 10% Pd/C (50% wet, 40 mg, 0.0187 mmol) was added to the flask, and the reaction mixture was evacuated and back-filled with nitrogen (2 times) and was then evacuated and back-filled with hydrogen (4 times). The reaction mixture was stirred under a hydrogen balloon for 1.5 h. The reaction mixture was evacuated and back-filled with nitrogen (4 times). Celite® was added, and the reaction mixture was stirred for approximately 10 min and was filtered through Celite®. The wet pad was rinsed with EtOH (5 mL×2), and the combined organic layers were concentrated to give the crude title compound (166.4 mg, 100%), which was used without further purification. MS (ESI+) m/z: 730.26 [M+H]+, formate salt, 1H NMR (400 MHz, Methanol-d) δ 8.48 (s, 3H), 4.46 (dd, 1H), 4.34-4.06 (m, 2H), 3.80-3.67 (m, 1H), 3.55-3.34 (m, 5H), 3.25-3.05 (m, 2H), 3.04-2.86 (m, 6H), 2.86-2.74 (m, 10H), 2.18-1.90 (m, 4H), 1.85-1.57 (m, 7H), 1.57-1.46 (m, 6H), 1.43-1.23 (m, 13H), 1.09-0.90 (m, 6H).

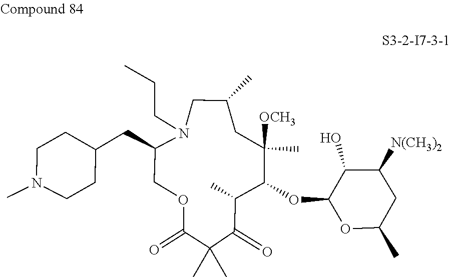

(3R,6R,8R,9R,10R)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-3-((1-methylpiperidin-4-yl)methyl)-4-propyl-1-oxa-4-azacyclotridecane-11,13-dione (S3-2-I7-3-1)

Prepared according to the methods of S3-2-I4-1-2 from S3-1-I7-3 and formaldehyde to provide the title compound as a formate salt. MS (ESI+) m/z: 640.33 [M+H]+; 1H NMR (400 MHz, Methanol-d) δ 8.48 (s, 3H), 4.46 (dd, 1H), 4.34-4.06 (m, 2H), 3.80-3.67 (m, 1H), 3.55-3.34 (m, 5H), 3.25-3.05 (m, 2H), 3.04-2.86 (m, 6H), 2.86-2.74 (m, 10H), 2.18-1.90 (m, 4H), 1.85-1.57 (m, 7H), 1.57-1.46 (m, 6H), 1.43-1.23 (m, 13H), 1.09-0.90 (m, 6H).

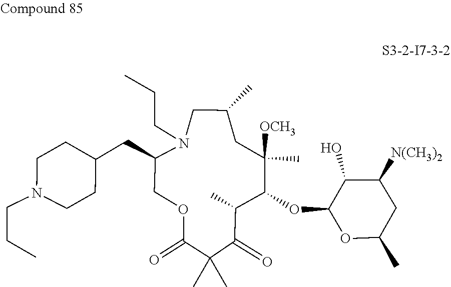 [SMILES: CCCN1CCC(C[C@@H]2COC(=O)C(C)(C)C(=O)[C@H](C)[C@@H](O[C@@H]3O[C@H](C)C[C@H](N(C)C)[C@H]3O)[C@](C)(OC)C[C@@H](C)CN2CCC)CC1]

(3R,6R,8R,9R,10R)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propyl-3-((1-propylpiperidin-4-yl)methyl)-1-oxa-4-azacyclotridecane-11,13-dione (S3-2-I7-3-2)

Prepared according to the methods of S3-2-I4-1-2 from S3-1-I7-3 and propionaldehyde to provide the title compound as a formate salt. MS (ESI+) m/z: 668.38 [M+H]+; 1H NMR (400 MHz, Methanol-d) δ8.51 (s, 3H), 4.46 (d, 1H), 4.26-3.96 (m, 2H), 3.79-3.66 (m, 1H), 3.62-3.49 (m, 3H), 3.49-3.34 (m, 3H), 3.05-2.96 (m, 3H), 2.96-2.83 (m, 5H), 2.83-2.73 (m, 7H), 2.15-1.91 (m, 4H), 1.82-1.70 (m, 3H), 1.70-1.43 (m, 11H), 1.41-1.22 (m, 13H), 1.07-0.83 (m, 9H).

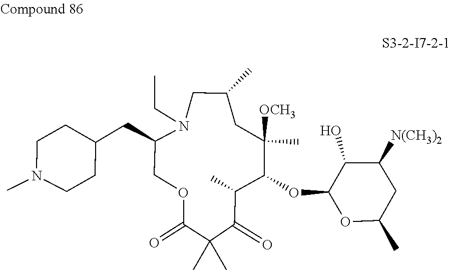

(3R,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-3-((1-methylpiperidin-4-yl)methyl)-1-oxa-4-azacyclotridecane-11,13-dione (S3-2-I7-2-1)

Prepared according to the methods of S3-2-I4-1-2 from S3-1-I7-2 and formaldehyde to provide the title compound as a formate salt. MS (ESI+) m/z: 209.5 [M+3H]3+, 313.8 [M+2H]2+, 626.5 [M+H]+; 1H NMR (400 MHz, Methanol-d4) δ 8.50 (s, 2H), 4.41 (d, 1H), 4.14 (s, 1H), 3.68 (m, 1H), 3.46-3.25 (m, 5H), 2.95-2.80 (m, 5H), 2.75 (d, 6H), 2.07-1.94 (m, 3H), 1.89 (d, 1H), 1.52 (s, 1H), 1.44 (d, 3H), 1.30 (m, 12H), 1.21 (s, 2H), 1.00-0.93 (m, 2H).

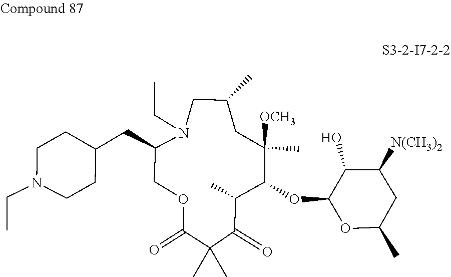 [SMILES: CCN1CCC(C[C@@H]2COC(=O)C(C)(C)C(=O)[C@H](C)[C@@H](O[C@@H]3O[C@H](C)C[C@H](N(C)C)[C@H]3O)[C@](C)(OC)C[C@@H](C)CN2CC)CC1]

(3R,6R,8R,9R,10R)-9-(((2S,3R,4S,6R)-4-(dimethylamino)-3-hydroxy-6-methyltetrahydro-2H-pyran-2-yl)oxy)-4-ethyl-3-((1-ethylpiperidin-4-yl)methyl)-8-methoxy-6,8,10,12,12-pentamethyl-1-oxa-4-azacyclotridecane-11,13-dione (S3-2-I7-2-2)

Prepared according to the methods of S3-2-I4-1-2 from S3-1-I7-2 and acetaldehyde to provide the title compound as a formate salt. MS (ESI+) m/z: 640.35 [M+H]+; 1H NMR (400 MHz, Methanol-d) δ8.54 (s, 3H), 4.46 (d, 1H), 4.37-3.97 (m, 2H), 3.79-3.65 (m, 1H), 3.64-3.40 (m, 5H), 3.40-3.33 (m, 1H), 3.16-3.03 (m, 3H), 3.03-2.81 (m, 6H), 2.81-2.70 (m, 8H), 2.16-1.84 (m, 5H), 1.57-1.41 (m, 8H), 1.41-1.08 (m, 20H), 1.08-0.81 (m, 4H).

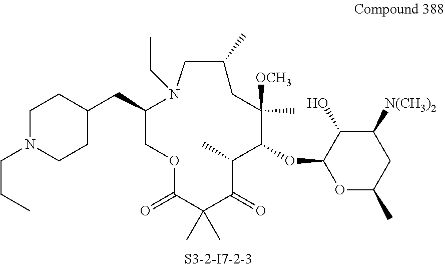 [SMILES: CCCN1CCC(C[C@@H]2COC(=O)C(C)(C)C(=O)[C@H](C)[C@@H](O[C@@H]3O[C@H](C)C[C@H](N(C)C)[C@H]3O)[C@](C)(OC)C[C@@H](C)CN2CC)CC1]

(3R,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-3-((1-propylpiperidin-4-yl)methyl)-1-oxa-4-azacyclotridecane-11,13-dione (S3-2-I7-2-3)

Prepared according to the methods of S3-2-I4-1-2 from S3-1-I7-2 and propionaldehyde to provide the title compound as a formate salt. MS (ESI+) m/z: 654.30 [M+H]+; 1H NMR (400 MHz, Methanol-d) δ8.51 (s, 2H), 4.46 (d, 1H), 4.36-3.90 (m, 2H), 3.78-3.65 (m, 1H), 3.62-3.39 (m, 5H), 3.39-3.33 (m, 1H), 3.07-2.82 (m, 8H), 2.82-2.67 (m, 8H), 2.14-1.87 (m, 4H), 1.82-1.60 (m, 5H), 1.60-1.41 (m, 8H), 1.41-1.24 (m, 14H), 1.24-1.07 (m, 3H), 1.07-0.81 (m, 6H).

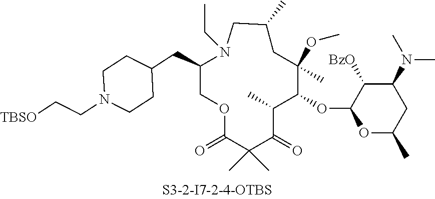

(2S,3R,4S,6R)-2-(((3R,6R,8R,9R,10R)-3-((1-(2-((tert-Butyldimethylsilyl)oxy)ethyl)piperidin-4-yl)methyl)-4-ethyl-8-methoxy-6,8,10,12,12-pentamethyl-11,13-dioxo-1-oxa-4-azacyclotridecan-9-yl)oxy)-4-(dimethylamino)-6-methyltetrahydro-2H-pyran-3-yl benzoate (S3-2-I7-2-4-OTBS)

S3-1-I7-2 (90 mg, 0.13 mmol) was dissolved in dry methylene chloride (2 mL). Acetic acid (0.021 mL, 0.38 mmol) and 2-((tert-butyldimethylsilyl)oxy)acetaldehyde (0.036 mL, 0.19 mmol) was added. Then NaBH(OAc)3 (53 mg, 0.25 mmol) was added to the reaction mixture In an portion. The reaction was allowed to stir at rt for 2 h at which point LC/MS showed full conversion. The reaction was quenched by adding saturated, aqueous NaHCO3(5 mL), and the aqueous layer was extracted with methylene chloride (3×10 mL). The combined extracts were dried over MgSO4, were filtered, and were concentrated. The residue was purified on 4 g of silica gel (elution with 0-10% MeOH— dichloromethane+0.5% of 30% aq NH4OH) to give the title compound (60 mg, 55%). MS (ESI+) m/z: 292.2 [M+3H]3+, 437.8 [M+2H]2+, 874.6 [M+H]+.

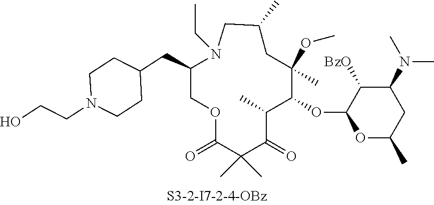

(2S,3R,4S,6R)-4-(Dimethylamino)-2-(((3R,6R,8R,9R,10R)-4-ethyl-3-((1-(2-hydroxyethyl)piperidin-4-yl)methyl)-8-methoxy-6,8,10,12,12-pentamethyl-11,13-dioxo-1-oxa-4-azacyclotridecan-9-yl)oxy)-6-methyltetrahydro-2H-pyran-3-yl benzoate (S3-2-I7-2-4-OBz)

S3-2-I7-2-4-OTBS (60 mg, 0.067 mmol) was dissolved in dry THE (2 mL) and TBAF (1M in THF, 0.20 mL, 0.020 mmol) was added at room temperature. The reaction mixture was stirred at rt for 2 h and was concentrated. The residue was purified on 4 g of silica gel (elution with 0-20% MeOH— dichloromethane+0.5% of 30% aq NH4OH) to give the title compound (46 mg, 88%). MS (ESI+) m/z: 254.2 [M+3H]3+, 380.8 [M+2H]2+, 760.5 [M+H]+.

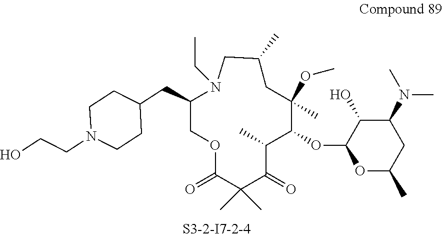 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@H]1CC1CCN(CCO)CC1]

(3R,6R,8R,9R,10R)-9-(((2S,3R,4S,6R)-4-(Dimethylamino)-3-hydroxy-6-methyltetrahydro-2H-pyran-2-yl)oxy)-4-ethyl-3-((1-(2-hydroxyethyl)piperidin-4-yl)methyl)-8-methoxy-6,8,10,12,12-pentamethyl-1-oxa-4-azacyclotridecane-11,13-dione (S3-2-I7-2-4)

Prepared by methanolysis of S3-2-I7-2-4-OTBS according to the methods of S3-2-I4-1-2. MS (ESI+) m/z: 219.5 [M+3H]3+, 328.8 [M+2H]2+, 656.5 [M+H]+; 1H NMR (400 MHz, Methanol-d4) δ 8.53 (s, 2H), 4.45 (d, 1H), 3.84 (t, 2H), 3.71 (ddd, 1H), 3.53-3.40 (m, 4H), 3.40-3.27 (m, 6H), 3.13 (d, 1H), 3.08 (s, 2H), 2.99-2.90 (m, 3H), 2.84 (s, 1H), 2.77 (s, 7H), 2.08-1.96 (m, 3H), 1.92 (d, 1H), 1.59 (s, 1H), 1.47 (dd, 5H), 1.39-1.27 (m, 12H), 0.97 (s, 2H).

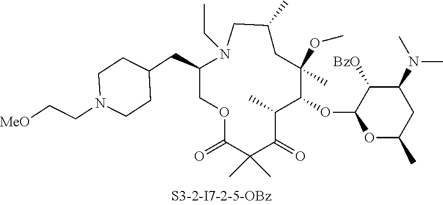

(2S,3R,4S,6R)-4-(Dimethylamino)-2-(((3R,6R,8R,9R,10R)-4-ethyl-8-methoxy-3-((1-(2-methoxyethyl)piperidin-4-yl)methyl)-6,8,10,12,12-pentamethyl-11,13-dioxo-1-oxa-4-azacyclotridecan-9-yl)oxy)-6-methyltetrahydro-2H-pyran-3-yl benzoate (S3-2-I7-2-5-OBz)

In a 8 mL vial was a solution of S3-2-I7-2-4-OBz in 1,2-dimethoxyethane (2 mL) precooled at −60° C. KHMDS (0.10 mL, 0.10 mmol) was added dropwise. The reaction mixture was stirred at −60° C. for 20 min. Then Me2SO4 (16 μL, 0.17 mmol) was added. The reaction mixture was allowed to warm to −15° C. LC/MS shows full conversion. The reaction was quenched by adding triethylamine (1 mL) and the resulting mixture was diluted with dichloromethane and saturated NaHCO3 was added. The aqueous layer was extracted with dichloromethane and the combined organic layers were dried over MgSO4, filtered and concentrated. The residue was purified on 4 g of silica gel (elution with 0-10% MeOH-dichloromethane+0.5% of 30% aq NH4OH) to give the title compound (22 mg, 82%). MS (ESI+) m/z: 258.8 [M+3H]3+, 387.8 [M+2H]2+, 774.5 [M+H]+.

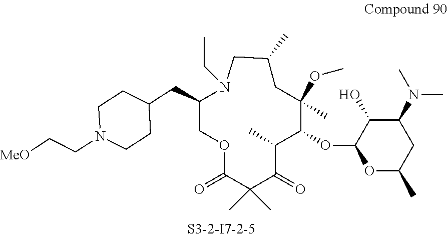 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@H]1CC1CCN(CCOC)CC1]

(3R,6R,8R,9R,10R)-9-(((2S,3R,4S,6R)-4-(Dimethylamino)-3-hydroxy-6-methyltetrahydro-2H-pyran-2-yl)oxy)-4-ethyl-8-methoxy-3-((1-(2-methoxyethyl)piperidin-4-yl)methyl)-6,8,10,12,12-pentamethyl-1-oxa-4-azacyclotridecane-11,13-dione (S3-2-I7-2-5)

Prepared by methanolysis of S3-2-I7-2-5-OBz according to the methods of S3-2-I4-1-2. MS (ESI+) m/z: 224.2 [M+3H]3+, 335.8 [M+2H]2+, 670.5 [M+H]+; 1H NMR (400 MHz, Methanol-d4) δ 8.53 (s, 2H), 4.45 (d, 1H), 4.14 (s, 1H), 3.69 (dt, 3H), 3.54-3.44 (m, 2H), 3.44-3.27 (m, 10H), 3.19 (d, 1H), 3.13 (s, 2H), 2.94 (s, 2H), 2.78 (d, 8H), 2.06-1.96 (m, 2H), 1.89 (d, 1H), 1.54 (s, 4H), 1.51-1.40 (m, 3H), 1.39-1.27 (m, 12H), 1.25-1.18 (m, 2H), 0.98 (s, 2H).

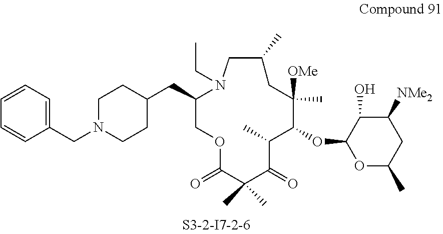

(3R,6R,8R,9R,10R)-3-((1-benzylpiperidin-4-yl)methyl)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-1-oxa-4-azacyclotridecane-11,13-dione (S3-2-I7-2-6)

Prepared according to the methods of S3-2-I4-1-2 from S3-1-I7-2 and benzaldehyde to provide the title compound as a formate salt. MS (ESI+) m/z: 702.5 [M+H]+; ¹H NMR (400 MHz, METHANOL-d4) δ=7.36 (br d, 5H), 4.42 (br s, 1H), 4.34-3.85 (m, 3H), 3.82-3.54 (m, 4H), 3.37 (br s, 2H), 3.19-2.95 (m, 3H), 2.83 (br s, 3H), 2.65 (br d, 7H), 2.39-2.07 (m, 3H), 2.04-1.69 (m, 5H), 1.60-1.21 (m, 22H), 1.07 (br s, 3H), 0.86 (br s, 2H).

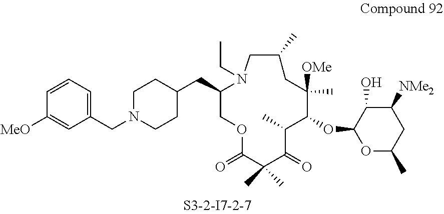

(3R,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-3-((1-(3-methoxybenzyl)piperidin-4-yl)methyl)-6,8,10,12,12-pentamethyl-1-oxa-4-azacyclotridecane-11,13-dione (S3-2-I7-2-7)

Prepared according to the methods of S3-2-I4-1-2 from S3-1-I7-2 and 3-methoxybenzaldehyde to provide the title compound as a formate salt. MS (ESI+) m/z: 732.5 [M+H]+; ¹H NMR (400 MHz, METHANOL-d₄) δ=8.55 (br s, 0.43H), 7.23 (t, 1H), 6.95-6.87 (m, 2H), 6.84 (dd, 1H), 4.44-4.30 (m, 2H), 4.09 (br d, 1H), 4.01-3.95 (m, 1H), 3.90 (br t, 1H), 3.79 (s, 3H), 3.74-3.65 (m, 1H), 3.60-3.56 (m, 1H), 3.53 (s, 2H), 3.11-3.04 (m, 1H), 2.95 (br t, 2H), 2.81 (s, 3H), 2.74-2.55 (m, 3H), 2.51-2.40 (m, 6H), 2.38-2.20 (m, 2H), 2.14-2.03 (m, 2H), 2.02-1.91 (m, 1H), 1.81 (br d, 2H), 1.76-1.65 (m, 2H), 1.53 (s, 3H), 1.46-1.20 (m, 20H), 1.14-0.95 (m, 5H), 0.84 (br d, 3H).

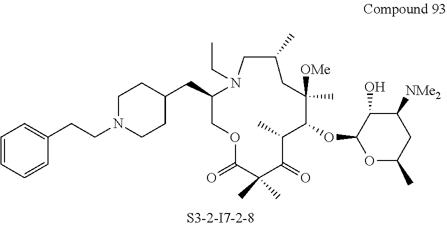

(3R,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-3-((1-phenethylpiperidin-4-yl)methyl)-1-oxa-4-azacyclotridecane-11,13-dione (S3-2-I7-2-8)

Prepared according to the methods of S3-2-I4-1-2 from S3-1-I7-2 and phenylacetaldehyde to provide the title compound as a formate salt. MS (ESI+) m/z: 716.5 [M+H]+; ¹H NMR (400 MHz, METHANOL-d₄) δ=8.55 (s, 1H), 7.33-7.26 (m, 2H), 7.25-7.16 (m, 3H), 4.60 (br s, 1H), 4.42 (br d, 1H), 4.29 (br s, 1H), 4.11 (br s, 1H), 4.04-3.88 (m, 2H), 3.77-3.59 (m, 2H), 3.39 (br s, 1H), 3.25-2.98 (m, 5H), 2.95-2.75 (m, 7H), 2.73-2.52 (m, 5H), 2.48-2.09 (m, 5H), 2.05-1.76 (m, 3H), 1.69 (br s, 1H), 1.54 (br s, 4H), 1.47-1.18 (m, 20H), 1.15-0.97 (m, 4H), 0.95-0.76 (m, 3H).

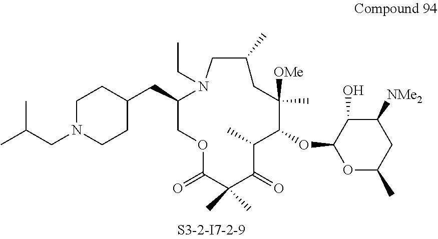

(3R,6R,8R,9R,10R)-9-(((2S,3R,4S,6R)-4-(dimethylamino)-3-hydroxy-6-methyltetrahydro-2H-pyran-2-yl)oxy)-4-ethyl-3-((1-isobutylpiperidin-4-yl)methyl)-8-methoxy-6,8,10,12,12-pentamethyl-1-oxa-4-azacyclotridecane-11,13-dione (S3-2-I7-2-9)

Prepared according to the methods of S3-2-I4-1-2 from S3-1-I7-2 and isobutyraldehyde to provide the title compound as a formate salt. MS (ESI+) m/z: 668.5 [M+H]+; ¹H NMR (400 MHz, METHANOL-d₄) δ=8.55 (br s, 1.33H), 4.56 (br s, 1H), 4.15-4.06 (m, 1H), 4.03-3.79 (m, 2H), 3.70 (br d, 1H), 3.65-3.61 (m, 1H), 3.52-3.43 (m, 1H), 3.12 (br d, 3H), 2.97-2.89 (m, 2H), 2.81 (br s, 2H), 2.67 (br dd, 3H), 2.52-2.20 (m, 12H), 2.07-1.61 (m, 8H), 1.53 (s, 3H), 1.50-1.21 (m, 22H), 1.06 (br d, 3H), 0.96 (br d, 7H), 0.85 (br d, 3H).

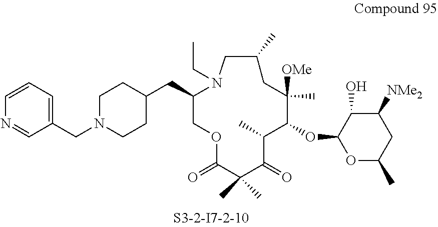

(3R,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-3-((1-(pyridin-3-ylmethyl)piperidin-4-yl)methyl)-1-oxa-4-azacyclotridecane-11,13-dione (S3-2-I7-2-10)

Prepared according to the methods of S3-2-I4-1-2 from S3-1-I7-2 and nicotinaldehyde to provide the title compound as a formate salt. MS (ESI+) m/z: 703.5 [M+H]+; ¹H NMR (400 MHz, METHANOL-d₄) δ=8.55 (s, 0.56H), 8.50 (d, 1H), 8.47-8.42 (m, 1H), 7.84 (br d, 1H), 7.42 (dd, 1H), 4.40 (br d, 1H), 4.09 (br d, 1H), 4.03-3.79 (m, 2H), 3.76-3.54 (m, 4H), 3.35 (br s, 1H), 3.15-2.86 (m, 5H), 2.81 (br s, 2H), 2.72-2.35 (m, 9H), 2.32-2.18 (m, 1H), 2.16-1.61 (m, 6H), 1.53 (s, 3H), 1.48-1.18 (m, 19H), 1.16-0.94 (m, 4H), 0.84 (br d, 2H).

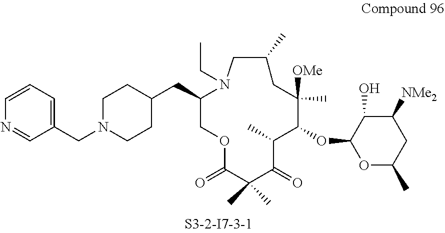

(3R,6R,8R,9R,10R)-3-((1-benzylpiperidin-4-yl)methyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propyl-1-oxa-4-azacyclotridecane-11,13-dione (S3-2-I7-3-1)

Prepared according to the methods of S3-2-I4-1-2 from S3-1-I7-3 and benzaldehyde to provide the title compound as a formate salt. MS (ESI+) m/z: 716.5 [M+H]+; ¹H NMR (400 MHz, METHANOL-d4) δ=8.53 (br s, 1H), 7.37 (br s, 5H), 4.43 (br d, 1H), 4.24-4.09 (m, 1H), 4.02-3.93 (m, 1H), 3.91-3.81 (m, 1H), 3.73-3.55 (m, 5H), 3.48-3.35 (m, 2H), 3.07 (br d, 6H), 2.80 (br s, 3H), 2.74-2.48 (m, 9H), 2.41-2.29 (m, 3H), 2.02-1.83 (m, 4H), 1.77 (br d, 3H), 1.56-1.20 (m, 26H), 1.04-0.70 (m, 7H).

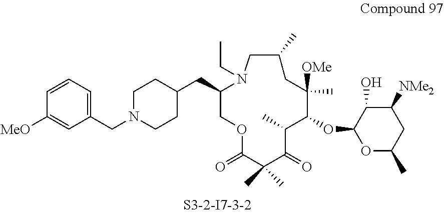

(3R,6R,8R,9R,10R)-9-(((2S,3R,4S,6R)-4-(dimethylamino)-3-hydroxy-6-methyltetrahydro-2H-pyran-2-yl)oxy)-8-methoxy-3-((1-(3-methoxybenzyl)piperidin-4-yl)methyl)-6,8,10,12,12-pentamethyl-4-propyl-1-oxa-4-azacyclotridecane-11,13-dione (S3-2-I7-3-2)

Prepared according to the methods of S3-2-I4-1-2 from S3-1-I7-3 and 3-methoxybenzaldehyde to provide the title compound as a formate salt. MS (ESI+) m/z: 746.5 [M+H]+; ¹H NMR (400 MHz, METHANOL-d₄) δ=8.55 (br s, 0.67H), 7.25 (t, 1H), 6.96-6.89 (m, 2H), 6.86 (dd, 1H), 4.45-4.35 (m, 1H), 4.18 (br d, 1H), 3.97 (br d, 1H), 3.87 (br t, 1H), 3.80 (s, 3H), 3.72-3.50 (m, 4H), 3.36 (br d, 1H), 3.12-3.03 (m, 1H), 2.98 (br t, 3H), 2.79 (s, 3H), 2.63-2.42 (m, 9H), 2.26-2.07 (m, 3H), 2.05-1.94 (m, 1H), 1.85 (br t, 2H), 1.78-1.59 (m, 3H), 1.51 (s, 3H), 1.49-1.38 (m, 4H), 1.37-1.20 (m, 18H), 1.05-0.88 (m, 5H), 0.83 (br d, 3H).

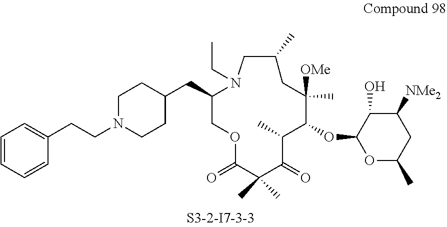

(3R,6R,8R,9R,10R)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-3-((1-phenethylpiperidin-4-yl)methyl)-4-propyl-1-oxa-4-azacyclotridecane-11,13-dione (S3-2-I7-3-3)

Prepared according to the methods of S3-2-I4-1-2 from S3-1-I7-3 and phenylacetaldehyde to provide the title compound as a formate salt. MS (ESI+) m/z: 730.5 [M+H]+; ¹H NMR (400 MHz, METHANOL-d₄) δ=8.56 (br s, 0.56H), 7.35-7.15 (m, 5H), 4.39 (br d, 1H), 4.20 (br d, 1H), 3.97 (br d, 1H), 3.88 (br t, 1H), 3.75-3.54 (m, 2H), 3.11 (br s, 3H), 2.99-2.75 (m, 7H), 2.72-2.37 (m, 11H), 2.24 (br d, 3H), 2.08-1.75 (m, 5H), 1.68 (br s, 2H), 1.52 (s, 3H), 1.47 (td, 4H), 1.40-1.21 (m, 16H), 1.01 (br dd, 1H), 0.97-0.90 (m, 3H), 0.84 (br d, 2H).

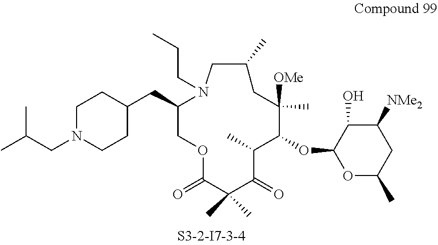

(3R,6R,8R,9R,10R)-9-(((2S,3R,4S,6R)-4-(dimethylamino)-3-hydroxy-6-methyltetrahydro-2H-pyran-2-yl)oxy)-3-((1-isobutylpiperidin-4-yl)methyl)-8-methoxy-6,8,10,12,12-pentamethyl-4-propyl-1-oxa-4-azacyclotridecane-11,13-dione (S3-2-I7-3-4)

Prepared according to the methods of S3-2-I4-1-2 from S3-1-I7-3 and isobutyraldehyde to provide the title compound as a formate salt. MS (ESI+) m/z: 682.5 [M+H]+; ¹H NMR (400 MHz, METHANOL-d₄) δ=8.55 (s, 1.28H), 4.42 (br d, 1H), 4.17 (br s, 1H), 3.98 (br d, 1H), 3.89 (br t, 1H), 3.67 (br d, 2H), 3.48 (br s, 2H), 3.27-3.17 (m, 1H), 3.08 (br s, 2H), 2.80 (br s, 6H), 2.69-2.33 (m, 9H), 2.23 (br s, 1H), 2.09-1.77 (m, 5H), 1.68 (br s, 1H), 1.59-1.21 (m, 23H), 1.10-0.76 (m, 13H).

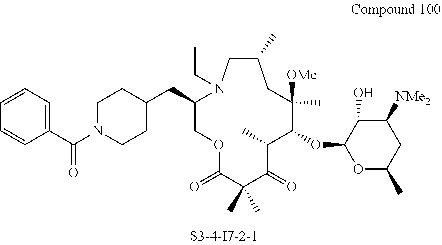

(3R,6R,8R,9R,10R)-3-((1-benzoylpiperidin-4-yl)methyl)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-1-oxa-4-azacyclotridecane-11,13-dione (S3-4-I7-2-1)

Prepared according to the methods of S3-4-I4-1-1 from S3-1-I7-2 and benzoyl chloride to provide the title compound as a formate salt. MS (ESI+) m/z: 716.5 [M+H]+; ¹H NMR (400 MHz, METHANOL-d4) δ=7.50-7.34 (m, 5H), 4.75-4.64 (m, 1H), 4.38 (br d, 1H), 4.12 (br d, 1H), 4.02-3.86 (m, 2H), 3.78-3.65 (m, 2H), 3.58 (br dd, 1H), 3.26-3.20 (m, 1H), 3.18-3.02 (m, 2H), 2.96-2.84 (m, 1H), 2.81 (s, 3H), 2.77-2.58 (m, 4H), 2.40 (s, 6H), 2.27 (br d, 1H), 2.04-1.93 (m, 1H), 1.90-1.74 (m, 3H), 1.73-1.58 (m, 3H), 1.55-1.50 (m, 3H), 1.48-1.41 (m, 1H), 1.38-1.17 (m, 16H), 1.11-0.93 (m, 5H), 0.85 (br d, 3H).

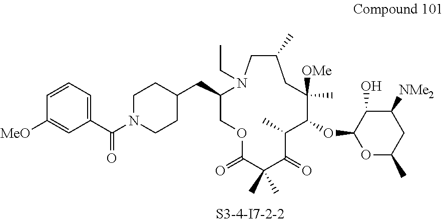

(3R,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-3-((1-(3-methoxybenzoyl)piperidin-4-yl)methyl)-6,8,10,12,12-pentamethyl-1-oxa-4-azacyclotridecane-11,13-dione (S3-4-I7-2-2)

Prepared according to the methods of S3-4-I4-1-1 from S3-1-I7-2 and 3-methoxybenzoyl chloride to provide the title compound as a formate salt. MS (ESI+) m/z: 746.5 [M+H]+; ¹H NMR (400 MHz, METHANOL-d₄) δ=8.55 (br s, 0.19H), 7.37 (t, 1H), 7.02 (br d, 1H), 6.98-6.89 (m, 2H), 4.62 (br s, 1H), 4.39 (br d, 1H), 4.12 (br d, 1H), 4.01-3.89 (m, 2H), 3.83 (s, 3H), 3.71 (br s, 2H), 3.60 (br dd, 1H), 3.19-3.03 (m, 2H), 2.95-2.74 (m, 5H), 2.73-2.57 (m, 3H), 2.47 (br s, 6H), 2.27 (br d, 1H), 2.06-1.91 (m, 1H), 1.82-1.57 (m, 5H), 1.53 (s, 3H), 1.47 (br s, 1H), 1.42-1.20 (m, 16H), 1.13-0.98 (m, 4H), 0.93-0.78 (m, 3H).

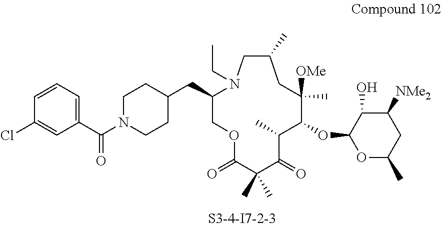

(3R,6R,8R,9R,10R)-3-((1-(3-chlorobenzoyl)piperidin-4-yl)methyl)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-1-oxa-4-azacyclotridecane-11,13-dione (S3-4-I7-2-3)

Prepared according to the methods of S3-4-I4-1-1 from S3-1-I7-2 and 3-chlorobenzoyl chloride to provide the title compound as a formate salt. MS (ESI+) m/z: 750.4 [M+H]+; ¹H NMR (400 MHz, METHANOL-d4) δ=8.56 (br s, 1H), 7.54-7.40 (m, 3H), 7.34 (br d, 1H), 4.60 (br s, 1H), 4.40 (br d, 1H), 4.20-4.04 (m, 1H), 4.02-3.86 (m, 2H), 3.79-3.53 (m, 3H), 3.22-3.01 (m, 3H), 2.96-2.77 (m, 5H), 2.75-2.56 (m, 3H), 2.46 (br s, 6H), 2.35-2.22 (m, 1H), 2.00 (br dd, 2H), 1.82 (br d, 2H), 1.74-1.60 (m, 3H), 1.59-1.43 (m, 1H), 1.42-1.15 (m, 17H), 1.15-1.05 (m, 3H), 1.03-0.96 (m, 1H), 0.86 (br d, 3H).

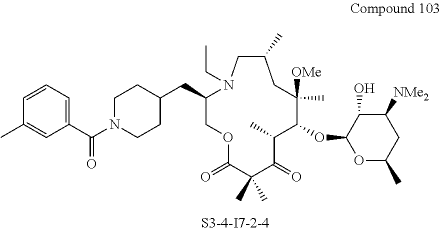

(3R,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-3-((1-(3-methylbenzoyl)piperidin-4-yl)methyl)-1-oxa-4-azacyclotridecane-11,13-dione (S3-4-I7-2-4)

Prepared according to the methods of S3-4-I4-1-1 from S3-1-I7-2 and 3-methylbenzoyl chloride to provide the title compound as a formate salt. MS (ESI+) m/z: 730.5 [M+H]+; ¹H NMR (400 MHz, METHANOL-d4) δ=7.40-7.25 (m, 2H), 7.24-7.12 (m, 2H), 4.71-4.53 (m, 2H), 4.38 (d, 1H), 4.11 (br d, 1H), 4.01-3.88 (m, 2H), 3.81-3.64 (m, 2H), 3.58 (td, 1H), 3.25-3.21 (m, 1H), 3.17-3.03 (m, 2H), 2.81 (s, 4H), 2.75-2.57 (m, 4H), 2.45-2.18 (m, 10H), 2.06-1.93 (m, 1H), 1.83-1.74 (m, 2H), 1.70-1.42 (m, 7H), 1.37-0.95 (m, 22H), 0.86 (br d, 3H).

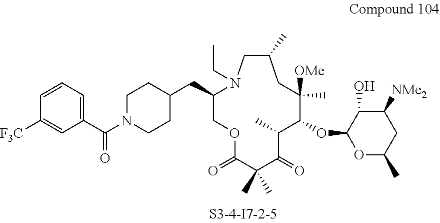

(3R,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-3-((1-(3-(trifluoromethyl)benzoyl)piperidin-4-yl)methyl)-1-oxa-4-azacyclotridecane-11,13-dione (S3-4-I7-2-5)

Prepared according to the methods of S3-4-I4-1-1 from S3-1-I7-2 and 3-trifluoromethylbenzoyl chloride to provide the title compound as a formate salt. MS (ESI+) m/z: 784.4 [M+H]+; ¹H NMR (400 MHz, METHANOL-d4) δ=7.78 (br s, 1H), 7.71 (s, 1H), 7.67 (d, 2H), 4.63 (br s, 1H), 4.37 (br d, 1H), 4.11 (br d, 1H), 4.01-3.87 (m, 2H), 3.76-3.51 (m, 3H), 3.27-3.23 (m, 1H), 3.20-3.04 (m, 2H), 3.0-2.85 (m, 1H), 2.80 (s, 3H), 2.42 (s, 6H), 2.27 (br d, 1H), 2.05-1.91 (m, 1H), 1.89-1.74 (m, 2H), 1.73-1.57 (m, 2H), 1.55-1.42 (m, 4H), 1.40-1.13 (m, 18H), 1.10-0.94 (m, 4H), 0.92-0.75 (m, 3H).

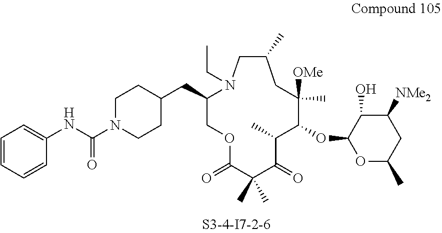

4-(((3R,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-11,13-dioxo-1-oxa-4-azacyclotridecan-3-yl)methyl)-N-phenylpiperidine-1-carboxamide (S3-4-I7-2-6)

Prepared according to the methods of S3-4-I4-1-1 from S3-1-I7-2 and phenyl isocyanate to provide the title compound as a formate salt. MS (ESI+) m/z: 731.5 [M+H]+; ¹H NMR (400 MHz, METHANOL-d₄) δ=8.56 (s, 0.31H), 7.36-7.31 (m, 2H), 7.29-7.21 (m, 2H), 7.01 (t, 1H), 4.39 (d, 1H), 4.26-4.07 (m, 3H), 4.03-3.89 (m, 2H), 3.79-3.53 (m, 3H), 3.20-3.09 (m, 1H), 2.97-2.85 (m, 3H), 2.82 (s, 3H), 2.75-2.57 (m, 3H), 2.45 (s, 6H), 2.40-2.34 (m, 1H), 2.28 (br d, 1H), 2.06-1.95 (m, 1H), 1.91-1.74 (m, 4H), 1.69 (br s, 1H), 1.60-1.43 (m, 6H), 1.41-1.20 (m, 18H), 1.13-0.96 (m, 5H), 0.85 (br d, 3H).

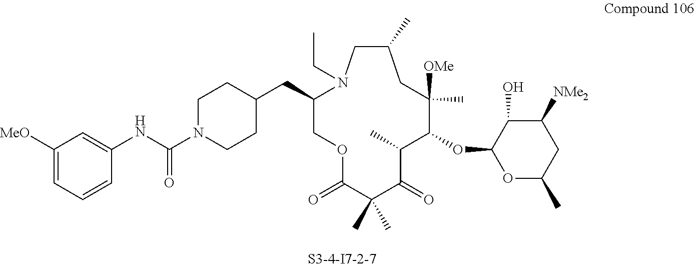

4-(((3R,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-11,13-dioxo-1-oxa-4-azacyclotridecan-3-yl)methyl)-N-(3-methoxyphenyl)piperidine-1-carboxamide (S3-4-I7-2-7)

Prepared according to the methods of S3-4-I4-1-1 from S3-1-I7-2 and 3-methoxyphenyl isocyanate to provide the title compound as a formate salt. MS (ESI+) m/z: 761.5 [M+H]+; ¹H NMR (399 MHz, METHANOL-d4) δ=7.15 (t, 1H), 7.04 (t, 1H), 6.91 (td, 1H), 6.59 (dd, 1H), 4.45-4.36 (m, 1H), 4.26-4.06 (m, 3H), 4.04-3.86 (m, 2H), 3.78 (s, 3H), 3.74-3.68 (m, 1H), 3.65-3.55 (m, 1H), 3.40-3.34 (m, 1H), 3.22-3.10 (m, 1H), 2.96-2.80 (m, 6H), 2.77-2.60 (m, 3H), 2.57-2.43 (m, 5H), 2.41-2.35 (m, 1H), 2.29 (br d, 1H), 2.07-1.96 (m, 1H), 1.93-1.73 (m, 3H), 1.72-1.60 (m, 1H), 1.58-1.44 (m, 5H), 1.41-1.16 (m, 17H), 1.11-0.98 (m, 4H), 0.86 (br d, 3H).

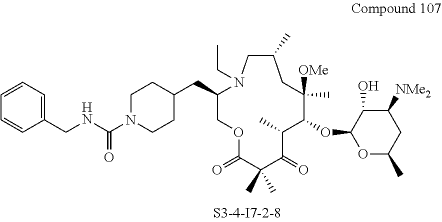

N-benzyl-4-(((3R,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-11,13-dioxo-1-oxa-4-azacyclotridecan-3-yl)methyl)piperidine-1-carboxamide (S3-4-I7-2-8)

Prepared according to the methods of S3-4-I4-1-1 from S3-1-I7-2 and benzyl isocyanate to provide the title compound as a formate salt. MS (ESI+) m/z: 745.5 [M+H]+; ¹H NMR (400 MHz, METHANOL-d4) δ=7.34-7.27 (m, 4H), 7.26-7.18 (m, 1H), 4.40 (s, 1H), 4.37 (s, 2H), 4.19-3.89 (m, 6H), 3.79-3.67 (m, 1H), 3.59 (br dd, 1H), 3.31-3.23 (m, 1H), 3.13 (td, 1H), 2.90-2.76 (m, 6H), 2.75-2.59 (m, 5H), 2.38 (s, 7H), 2.30 (br d, 1H), 2.00 (br dd, 1H), 1.90-1.62 (m, 5H), 1.56 (s, 4H), 1.51-1.41 (m, 3H), 1.36 (s, 3H), 1.32-1.22 (m, 13H), 1.20-1.13 (m, 2H), 1.08 (br t, 4H), 1.03-0.96 (m, 1H), 0.87 (d, 3H).

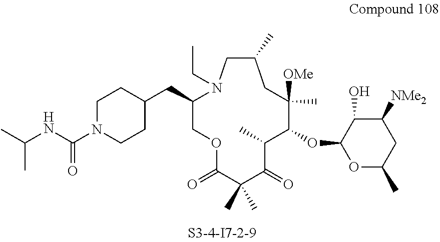

4-(((3R,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-11,13-dioxo-1-oxa-4-azacyclotridecan-3-yl)methyl)-N-isopropylpiperidine-1-carboxamide (S3-4-I7-2-9)

Prepared according to the methods of S3-4-I4-1-1 from S3-1-I7-2 and isopropyl isocyanate to provide the title compound as a formate salt. MS (ESI+) m/z: 697.5 [M+H]+; ¹H NMR (400 MHz, METHANOL-d4) δ=4.38 (d, 1H), 4.11 (br dd, 1H), 4.07-3.83 (m, 5H), 3.76-3.65 (m, 1H), 3.58 (br dd, 1H), 3.30-3.24 (m, 1H), 3.09 (br d, 1H), 2.81 (s, 3H), 2.78-2.55 (m, 7H), 2.38 (s, 6H), 2.28 (br d, 1H), 1.98 (br dd, 1H), 1.71 (br d, 4H), 1.58-1.51 (m, 3H), 1.50-1.38 (m, 2H), 1.34 (s, 3H), 1.31-1.20 (m, 11H), 1.19-1.10 (m, 8H), 1.09-1.03 (m, 4H), 1.02-0.97 (m, 1H), 0.85 (d, 3H).

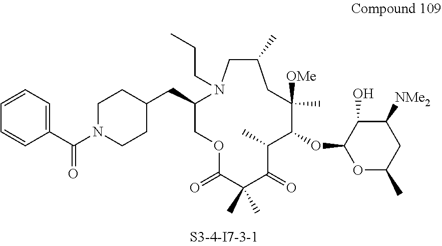

(3R,6R,8R,9R,10R)-3-((1-benzoylpiperidin-4-yl)methyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propyl-1-oxa-4-azacyclotridecane-11,13-dione (S3-4-I7-3-1)

Prepared according to the methods of S3-4-I4-1-1 from S3-1-I7-3 and benzoyl chloride to provide the title compound as a formate salt. MS (ESI+) m/z: 730.5 [M+H]+; ¹H NMR (400 MHz, METHANOL-d₄) δ=8.54 (s, 1.31H), 7.58-7.29 (m, 5H), 4.65 (br s, 1H), 4.45 (br d, 1H), 4.08 (br s, 2H), 3.85-3.64 (m, 3H), 3.62-3.38 (m, 2H), 3.34 (br s, 1H), 3.30-3.26 (m, 1H), 3.13 (br s, 2H), 3.01-2.81 (m, 4H), 2.76 (s, 6H), 2.10-1.95 (m, 3H), 1.93-1.13 (m, 27H), 0.98 (br s, 6H).

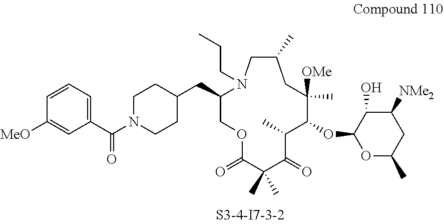

(3R,6R,8R,9R,10R)-9-(((2S,3R,4S,6R)-4-(dimethylamino)-3-hydroxy-6-methyltetrahydro-2H-pyran-2-yl)oxy)-8-methoxy-3-((1-(3-methoxybenzoyl)piperidin-4-yl)methyl)-6,8,10,12,12-pentamethyl-4-propyl-1-oxa-4-azacyclotridecane-11,13-dione (S3-4-I7-3-2)

Prepared according to the methods of S3-4-I4-1-1 from S3-1-I7-3 and 3-methoxybenzoyl chloride to provide the title compound as a formate salt. MS (ESI+) m/z: 760.5 [M+H]+; ¹H NMR (400 MHz, METHANOL-d₄) δ=8.55 (br s, 0.22H), 7.37 (t, 1H), 7.06-7.00 (m, 1H), 6.98-6.90 (m, 2H), 4.62 (br s, 1H), 4.38 (br d, 1H), 4.20 (br d, 1H), 4.02-3.94 (m, 1H), 3.89 (br t, 1H), 3.83 (s, 3H), 3.78-3.53 (m, 3H), 3.10 (br s, 2H), 2.79 (s, 5H), 2.65-2.49 (m, 3H), 2.43 (br s, 6H), 2.24 (br d, 1H), 2.09-1.73 (m, 4H), 1.73-1.55 (m, 3H), 1.54-1.41 (m, 6H), 1.38-1.06 (m, 16H), 1.06-0.97 (m, 1H), 0.94 (br t, 3H), 0.84 (br d, 3H).

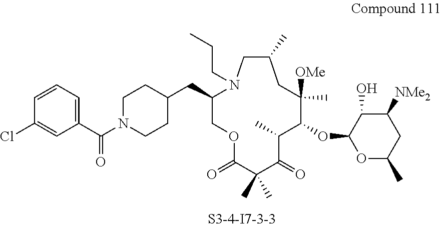

(3R,6R,8R,9R,10R)-3-((1-(3-chlorobenzoyl)piperidin-4-yl)methyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propyl-1-oxa-4-azacyclotridecane-11,13-dione (S3-4-I7-3-3)

Prepared according to the methods of S3-4-I4-1-1 from S3-1-I7-3 and 3-chlorobenzoyl chloride to provide the title compound as a formate salt. MS (ESI+) m/z: 764.4 [M+H]+; ¹H NMR (400 MHz, METHANOL-d₄) δ=8.54 (br s, 1.32H), 7.54-7.40 (m, 3H), 7.34 (br d, 1H), 4.63 (br s, 1H), 4.45 (br d, 1H), 4.07 (br s, 2H), 3.80-3.50 (m, 3H), 3.47-3.38 (m, 1H), 3.34 (br s, 1H), 3.30-3.26 (m, 1H), 3.15 (br s, 2H), 3.02-2.81 (m, 5H), 2.76 (s, 6H), 2.00 (br d, 2H), 1.85 (br s, 2H), 1.69 (br s, 2H), 1.61-1.12 (m, 23H), 0.98 (br s, 5H).

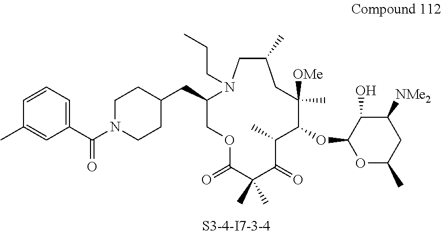

(3R,6R,8R,9R,10R)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-3-((1-(3-methylbenzoyl)piperidin-4-yl)methyl)-4-propyl-1-oxa-4-azacyclotridecane-11,13-dione (S3-4-I7-3-4)

Prepared according to the methods of S3-4-I4-1-1 from S3-1-I7-3 and 3-methylbenzoyl chloride to provide the title compound as a formate salt. MS (ESI+) m/z: 744.4 [M+H]+; ¹H NMR (400 MHz, METHANOL-d₄) δ=8.54 (br s, 1.18H), 7.38-7.27 (m, 2H), 7.25-7.13 (m, 2H), 4.71-4.56 (m, 1H), 4.45 (br d, 1H), 4.08 (br s, 2H), 3.85-3.49 (m, 3H), 3.47-3.39 (m, 1H), 3.49-3.38 (m, 1H), 3.34 (br s, 1H), 3.29-3.25 (m, 1H), 3.20-3.00 (m, 1H), 2.88 (br d, 4H), 2.81-2.67 (m, 6H), 2.39 (s, 3H), 2.00 (br d, 2H), 1.92-1.11 (m, 25H), 0.98 (br s, 6H).

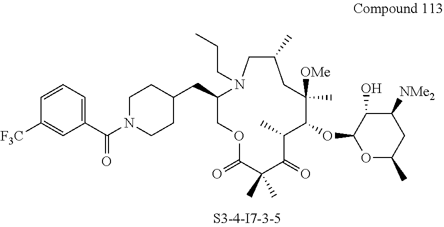

(3R,6R,8R,9R,10R)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propyl-3-((1-(3-(trifluoromethyl)benzoyl)piperidin-4-yl)methyl)-1-oxa-4-azacyclotridecane-11,13-dione (S3-4-I7-3-5)

Prepared according to the methods of S3-4-I4-1-1 from S3-1-I7-3 and 3-trifluoromethylbenzoyl chloride to provide the title compound as a formate salt. MS (ESI+) m/z: 798.5 [M+H]+; ¹H NMR (400 MHz, METHANOL-d₄) δ=8.54 (br s, 1.09H), 7.84-7.76 (m, 1H), 7.71 (s, 1H), 7.68 (d, 2H), 4.65 (br s, 1H), 4.44 (br d, 1H), 4.29-3.84 (m, 2H), 3.80-3.52 (m, 3H), 3.47-3.37 (m, 1H), 3.29-3.08 (m, 3H), 3.00-2.79 (m, 4H), 2.74 (s, 6H), 2.66-2.41 (m, 1H), 2.13 (br s, 1H), 2.07-1.95 (m, 2H), 1.94-1.13 (m, 27H), 1.09-0.61 (m, 6H).

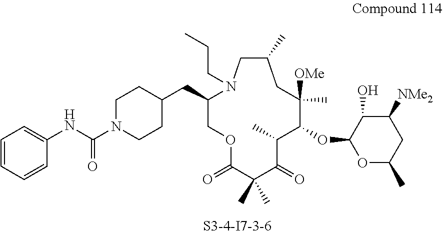

4-(((3R,6R,8R,9R,10R)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-11,13-dioxo-4-propyl-1-oxa-4-azacyclotridecan-3-yl)methyl)-N-phenylpiperidine-1-carboxamide (S3-4-I7-3-6)

Prepared according to the methods of S3-4-I4-1-1 from S3-1-I7-3 and phenyl isocyanate to provide the title compound as a formate salt. MS (ESI+) m/z: 745.5 [M+H]+; ¹H NMR (400 MHz, METHANOL-d4) δ=8.56 (s, 0.4H), 7.36-7.32 (m, 2H), 7.26 (t, 2H), 7.02 (t, 1H), 4.42 (br d, 1H), 4.31-4.11 (m, 3H), 4.07-3.82 (m, 2H), 3.76-3.57 (m, 2H), 3.50-3.34 (m, 2H), 3.20-3.06 (m, 1H), 3.04-2.84 (m, 4H), 2.81 (s, 3H), 2.67-2.43 (m, 10H), 2.25 (br d, 1H), 2.03 (br s, 1H), 1.94-1.83 (m, 2H), 1.79 (br d, 2H), 1.70 (br s, 1H), 1.59-1.39 (m, 9H), 1.39-1.26 (m, 15H), 1.25-1.11 (m, 3H), 1.09-0.90 (m, 5H), 0.85 (br d, 3H).

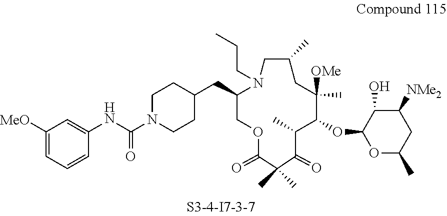

4-(((3R,6R,8R,9R,10R)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-11,13-dioxo-4-propyl-1-oxa-4-azacyclotridecan-3-yl)methyl)-N-(3-methoxyphenyl)piperidine-1-carboxamide (S3-4-I7-3-7)

Prepared according to the methods of S3-4-I4-1-1 from S3-1-I7-3 and 3-methoxyphenyl isocyanate to provide the title compound as a formate salt. MS (ESI+) m/z: 775.5 [M+H]+; ¹H NMR (400 MHz, METHANOL-d4) δ=7.14 (t, 1H), 7.03 (s, 1H), 6.91 (br d, 1H), 6.58 (dd, 1H), 4.38 (d, 1H), 4.29-4.09 (m, 3H), 3.98 (d, 1H), 3.90 (br t, 1H), 3.77 (s, 3H), 3.74-3.64 (m, 1H), 3.62-3.53 (m, 1H), 3.29-3.24 (m, 1H), 3.20-3.15 (m, 1H), 3.20-3.05 (m, 2H), 2.97-2.84 (m, 3H), 2.77-2.65 (m, 1H), 2.64-2.48 (m, 3H), 2.38 (s, 6H), 2.25 (br d, 1H), 2.10-1.97 (m, 1H), 1.88 (br d, 1H), 1.78 (br d, 2H), 1.69 (br s, 1H), 1.59-1.39 (m, 8H), 1.38-1.09 (m, 18H), 1.02 (br dd, 1H), 0.97-0.89 (m, 3H), 0.84 (d, 3H).

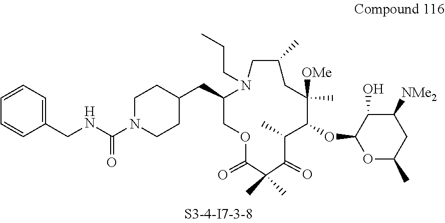

N-benzyl-4-(((3R,6R,8R,9R,10R)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-11,13-dioxo-4-propyl-1-oxa-4-azacyclotridecan-3-yl)methyl)piperidine-1-carboxamide (S3-4-I7-3-8)

Prepared according to the methods of S3-4-I4-1-1 from S3-1-I7-3 and benzyl isocyanate to provide the title compound as a formate salt. MS (ESI+) m/z: 759.5 [M+H]+; ¹H NMR (400 MHz, METHANOL-d4) δ=7.32-7.18 (m, 5H), 4.41-4.32 (m, 3H), 4.20 (br d, 1H), 4.11-4.00 (m, 2H), 4.00-3.94 (m, 1H), 3.93-3.84 (m, 1H), 3.68 (br dd, 1H), 3.58 (br dd, 1H), 3.29-3.21 (m, 1H), 3.20-3.05 (m, 1H), 2.85-2.76 (m, 5H), 2.75-2.47 (m, 5H), 2.38 (s, 6H), 2.31-2.18 (m, 1H), 2.07-1.97 (m, 1H), 1.86-1.61 (m, 5H), 1.56-1.39 (m, 8H), 1.36-1.08 (m, 17H), 1.05-0.91 (m, 4H), 0.88-0.82 (m, 3H).

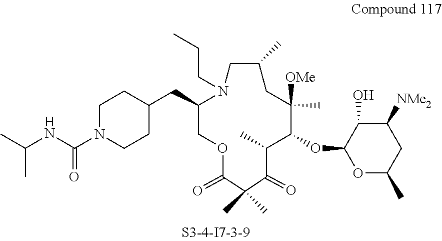

4-(((3R,6R,8R,9R,10R)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-11,13-dioxo-4-propyl-1-oxa-4-azacyclotridecan-3-yl)methyl)-N-isopropylpiperidine-1-carboxamide (S3-4-I7-3-9)

Prepared according to the methods of S3-4-I4-1-1 from S3-1-I7-3 and isopropyl isocyanate to provide the title compound as a formate salt. MS (ESI+) m/z: 711.5 [M+H]+; ¹H NMR (400 MHz, METHANOL-d₄) δ=4.37 (d, 1H), 4.19 (br d, 1H), 4.09-3.94 (m, 3H), 3.92-3.83 (m, 2H), 3.74-3.64 (m, 1H), 3.58 (br dd, 1H), 3.29-3.24 (m, 1H), 3.17-3.04 (m, 1H), 2.84-2.67 (m, 6H), 2.65-2.46 (m, 3H), 2.39 (s, 6H), 2.24 (br d, 1H), 2.08-1.95 (m, 1H), 1.87-1.61 (m, 4H), 1.52 (s, 3H), 1.50-1.37 (m, 4H), 1.34 (s, 3H), 1.32-1.20 (m, 11H), 1.14 (d, 7H), 1.09-0.97 (m, 2H), 0.94 (t, 3H), 0.84 (d, 3H).

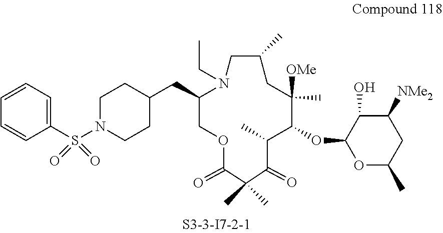

(3R,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-3-((1-(phenylsulfonyl)piperidin-4-yl)methyl)-1-oxa-4-azacyclotridecane-11,13-dione (S3-3-I7-2-1)

Prepared according to the methods of S3-4-I4-1-1 from S3-1-I7-2 and benzenesulfonyl chloride to provide the title compound as a formate salt. MS (ESI+) m/z: 752.5 [M+H]+; ¹H NMR (400 MHz, METHANOL-d4) δ=7.78 (br d, 2H), 7.72-7.65 (m, 1H), 7.65-7.57 (m, 2H), 4.63-4.53 (m, 0.4H), 4.45-4.34 (m, 1H), 4.31-4.16 (m, 1H), 4.13-4.01 (m, 1H), 3.96 (br d, 1H), 3.92-3.82 (m, 1H), 3.79-3.72 (m, 2H), 3.71-3.53 (m, 2H), 3.10-2.96 (m, 2H), 2.95-2.83 (m, 1H), 2.83-2.76 (m, 2H), 2.73-2.57 (m, 3H), 2.54-2.43 (m, 5H), 2.40-2.17 (m, 4H), 2.41-2.17 (m, 5H), 1.69-1.58 (m, 1H), 1.51 (s, 3H), 1.43-1.15 (m, 19H), 1.13-0.93 (m, 4H), 0.90-0.76 (m, 3H).

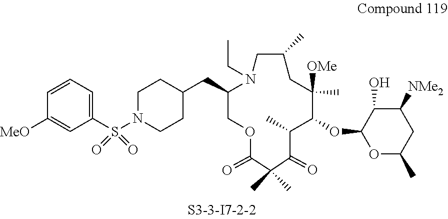

(3R,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-3-((1-((3-methoxyphenyl)sulfonyl)piperidin-4-yl)methyl)-6,8,10,12,12-pentamethyl-1-oxa-4-azacyclotridecane-11,13-dione (S3-3-I7-2-3)

Prepared according to the methods of S3-4-I4-1-1 from S3-1-I7-2 and 3-methoxybenzenesulfonyl chloride to provide the title compound as a formate salt. MS (ESI+) m/z: 782.4 [M+H]+; ¹H NMR (400 MHz, METHANOL-d4) δ=7.58-7.49 (m, 1H), 7.35 (d, 1H), 7.29-7.21 (m, 2H), 4.38 (d, 1H), 4.13-4.02 (m, 1H), 3.98 (d, 1H), 3.94-3.84 (m, 4H), 3.83-3.64 (m, 3H), 3.64-3.54 (m, 1H), 3.28 (br dd, 1H), 3.14-3.00 (m, 1H), 2.81 (s, 3H), 2.76-2.51 (m, 4H), 2.43-2.18 (m, 9H), 1.96 (br dd, 1H), 1.91-1.84 (m, 1H), 1.83-1.74 (m, 2H), 1.71-1.62 (m, 1H), 1.53 (s, 3H), 1.47-1.16 (m, 19H), 1.11-1.03 (m, 3H), 1.01-0.95 (m, 1H), 0.85 (d, 3H).

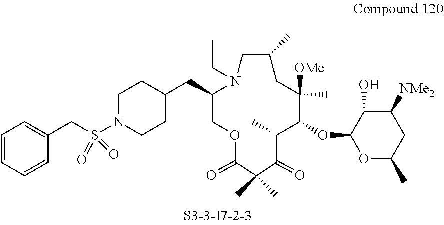

(3R,6R,8R,9R,10R)-3-((1-(benzylsulfonyl)piperidin-4-yl)methyl)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-1-oxa-4-azacyclotridecane-11,13-dione (S3-3-I7-2-3)

Prepared according to the methods of S3-4-I4-1-1 from S3-1-I7-2 and phenylmethanesulfonyl chloride to provide the title compound as a formate salt. MS (ESI+) m/z: 766.5 [M+H]+; METHANOL-d₄) δ=7.48-7.36 (m, 5H), 4.39 (d, 1H), 4.34 (s, 2H), 4.16-4.05 (m, 1H), 3.99 (d, 1H), 3.91 (t, 1H), 3.76-3.52 (m, 4H), 3.30-3.25 (m, 1H), 3.13-3.04 (m, 1H), 2.82 (s, 3H), 2.77-2.53 (m, 6H), 2.39 (s, 6H), 2.28 (br d, 1H), 1.98 (br dd, 1H), 1.85-1.63 (m, 4H), 1.55 (s, 3H), 1.49-1.22 (m, 17H), 1.16 (dt, 2H), 1.07 (t, 3H), 1.04-0.98 (m, 1H), 0.86 (d, 3H).

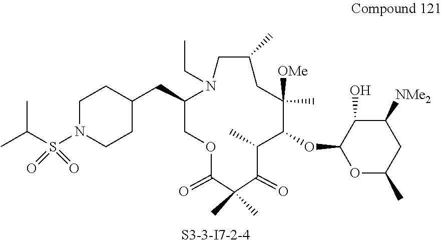

(3R,6R,8R,9R,10R)-9-(((2S,3R,4S,6R)-4-(dimethylamino)-3-hydroxy-6-methyltetrahydro-2H-pyran-2-yl)oxy)-4-ethyl-3-((1-(isopropylsulfonyl)piperidin-4-yl)methyl)-8-methoxy-6,8,10,12,12-pentamethyl-1-oxa-4-azacyclotridecane-11,13-dione (S3-3-I7-2-4)

Prepared according to the methods of S3-4-I4-1-1 from S3-1-I7-2 and isopropylsulfonyl chloride to provide the title compound as a formate salt. MS (ESI+) m/z: 718.5 [M+H]+; ¹H NMR (400 MHz, METHANOL-d4) δ=4.38 (d, 1H), 4.19-4.06 (m, 1H), 4.02-3.86 (m, 2H), 3.84-3.64 (m, 3H), 3.62-3.53 (m, 1H), 3.30-3.20 (m, 2H), 3.11 (br t, 1H), 2.92 (br t, 2H), 2.81 (s, 3H), 2.75-2.60 (m, 4H), 2.47-2.34 (m, 6H), 2.28 (br d, 1H), 2.07-1.93 (m, 1H), 1.92-1.83 (m, 1H), 1.78 (br d, 2H), 1.69 (br s, 1H), 1.54 (s, 3H), 1.50-1.41 (m, 2H), 1.40-1.14 (m, 22H), 1.07 (br t, 3H), 1.03-0.94 (m, 1H), 0.85 (br d, 3H).

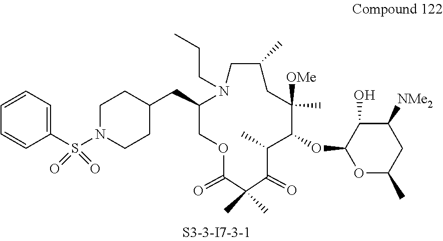

(3R,6R,8R,9R,10R)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-3-((1-(phenylsulfonyl)piperidin-4-yl)methyl)-4-propyl-1-oxa-4-azacyclotridecane-11,13-dione (S3-3-I7-3-1)

Prepared according to the methods of S3-4-I4-1-1 from S3-1-I7-3 and benzenesulfonyl chloride to provide the title compound as a formate salt. MS (ESI+) m/z: 766.4 [M+H]+; ¹H NMR (400 MHz, METHANOL-d4) δ=7.83-7.76 (m, 2H), 7.73-7.66 (m, 1H), 7.66-7.59 (m, 2H), 4.39 (d, 1H), 4.12 (br s, 1H), 3.97 (d, 1H), 3.90-3.72 (m, 3H), 3.70-3.53 (m, 2H), 3.30 (br d, 1H), 3.05 (br s, 1H), 2.79 (s, 3H), 2.64-2.52 (m, 2H), 2.50-2.39 (m, 7H), 2.38-2.25 (m, 2H), 2.21 (br d, 1H), 2.08-1.94 (m, 1H), 1.89 (br d, 1H), 1.84-1.72 (m, 2H), 1.66 (br s, 1H), 1.54-1.38 (m, 6H), 1.34 (s, 3H), 1.31-1.15 (m, 13H), 1.01 (br dd, 1H), 0.93 (t, 3H), 0.83 (d, 3H).

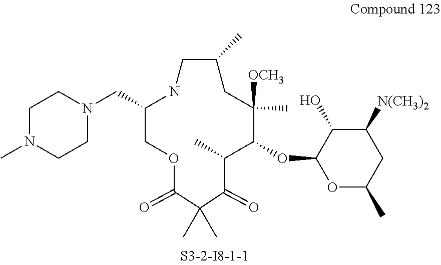 [SMILES: CO[C@]1(C)C[C@@H](C)CN[C@@H](CN2CCN(C)CC2)COC(=O)C(C)(C)C(=O)[C@H](C)[C@H]1O[C@@H]1O[C@H](C)C[C@H](N(C)C)[C@H]1O]

(3S,6R,8R,9R,10R)-9-(((2S,3R,4S,6R)-4-(Dimethylamino)-3-hydroxy-6-methyltetrahydro-2H-pyran-2-yl)oxy)-8-methoxy-4,6,8,10,12,12-hexamethyl-3-((4-methylpiperazin-1-yl)methyl)-1-oxa-4-azacyclotridecane-11,13-dione (S3-2-I8-1-1)

Prepared according to the methods of S3-2-I4-1-2 from S3-2-I8-1 and formaldehyde to provide 7.33 mg of the title compound as a formate salt. (ESI+) m/z: 205.04 [M+3H]3+, 307.01 [M+2H]2+, 613.01[M+H]+; 1H NMR (400 MHz, Methanol-d4) δ 8.53 (s, 2H), 4.58 (d, 1H), 4.45 (d, 1H), 4.34 (t, 1H), 4.26 (d, 1H), 3.90 (d, 1H), 3.77-3.67 (m, 1H), 3.54-3.33 (m, 3H), 3.19 (s, 1H), 3.02 (s, 3H), 2.82 (s, 3H), 2.76 (s, 9H), 2.73-2.54 (m, 7H), 2.45 (s, 3H), 2.18 (s, 1H), 2.04-1.95 (m, 1H), 1.57 (s, 3H), 1.53-1.43 (m, 1H), 1.39 (d, 6H), 1.33 (dd, 6H), 1.04 (d, 3H).

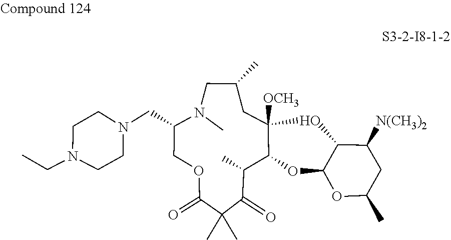 [SMILES: CCN1CCN(C[C@H]2COC(=O)C(C)(C)C(=O)[C@H](C)[C@H]3O[C@@H]4O[C@H](C)C[C@H](N(C)C)[C@H]4O[C@@]3(OC)C[C@@H](C)CN2C)CC1]

(3S,6R,8R,9R,10R)-9-(((2S,3R,4S,6R)-4-(dimethylamino)-3-hydroxy-6-methyltetrahydro-2H-pyran-2-yl)oxy)-3-((4-ethylpiperazin-1-yl)methyl)-8-methoxy-4,6,8,10,12,12-hexamethyl-1-oxa-4-azacyclotridecane-11,13-dione (S3-2-I8-1-2)

Prepared according to the methods of S3-2-I4-1-2 from S3-2-I8-1 and acetaldehyde to provide 6.82 mg of the title compound as a formate salt. (ESI+) m/z: 209.72 [M+3H]3+, 313.98 [M+2H]2+, 627.02 [M+H]+; 1H NMR (400 MHz, Methanol-d4) δ 8.53 (s, 2H), 4.58 (d, 1H), 4.46 (d, 1H), 4.34 (t, 1H), 4.25 (d, 1H), 3.83 (s, 1H), 3.77-3.68 (m, 1H), 3.54-3.36 (m, 2H), 3.14 (s, 1H), 3.02 (s, 3H), 2.92-2.78 (m, 6H), 2.76 (s, 8H), 2.74-2.64 (m, 5H), 2.60 (dd, 2H), 2.17 (s, 1H), 2.06-1.97 (m, 1H), 1.70 (s, 2H), 1.55 (s, 3H), 1.54-1.44 (m, 1H), 1.39 (d, 6H), 1.33 (dd, 6H), 1.20 (t, 3H), 1.03 (d, 3H).

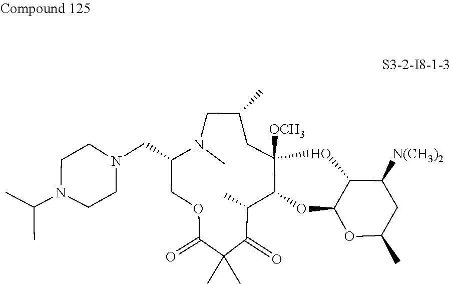 [SMILES: CO[C@]12C[C@@H](C)CN(C)[C@@H](CN3CCN(C(C)C)CC3)COC(=O)C(C)(C)C(=O)[C@H](C)[C@H]1O[C@@H]1O[C@H](C)C[C@H](N(C)C)[C@H]1O2]

(3S,6R,8R,9R,10R)-9-(((2S,3R,4S,6R)-4-(dimethylamino)-3-hydroxy-6-methyltetrahydro-2H-pyran-2-yl)oxy)-3-((4-isopropylpiperazin-1-yl)methyl)-8-methoxy-4,6,8,10,12,12-hexamethyl-1-oxa-4-azacyclotridecane-11,13-dione (S3-2-I8-1-3)

Prepared according to the methods of S3-2-I4-1-2 from S3-2-I8-1 and acetone to provide 11.7 mg of the title compound as a formate salt. (ESI+) m/z: 214.41 [M+3H]3+, 321.01 [M+2H]2+, 641.09 [M+H]+; 1H NMR (400 MHz, Methanol-d4) δ 8.59 (s, 2H), 4.64 (d, 1H), 4.51 (d, 1H), 4.39 (t, 1H), 4.29 (d, 1H), 3.85 (s, 1H), 3.82-3.73 (m, 1H), 3.58-3.43 (m, 2H), 3.27-3.12 (m, 2H), 3.05 (s, 7H), 2.82 (s, 14H), 2.66 (dd, 2H), 2.20 (s, 1H), 2.11-2.00 (m, 1H), 1.75 (s, 2H), 1.59 (s, 3H), 1.58-1.48 (m, 1H), 1.44 (s, 6H), 1.38 (dd, 6H), 1.30 (d, 6H), 1.08 (d, 3H).

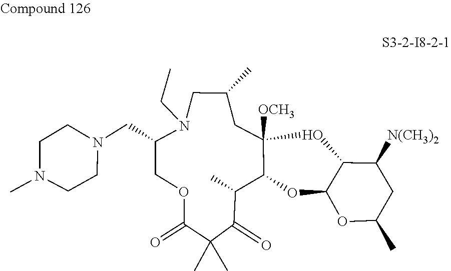

(3S,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-3-((4-methylpiperazin-1-yl)methyl)-1-oxa-4-azacyclotridecane-11,13-dione (S3-2-I8-2-1)

Prepared according to the methods of S3-2-I4-1-2 from S3-2-I8-2 and formaldehyde to provide 7.63 mg of the title compound as a formate salt. (ESI+) m/z: 209.74 [M+3H]3+, 314.01 [M+2H]2+, 627.11 [M+H]+; 1H NMR (400 MHz, Methanol-d4) δ 8.49 (s, 2H), 4.69 (s, 1H), 4.46 (d, 1H), 4.29 (s, 1H), 4.15 (d, 1H), 3.79-3.66 (m, 1H), 3.54 (s, 2H), 3.49-3.33 (m, 2H), 3.25-3.00 (m, 3H), 2.95 (s, 6H), 2.81 (s, 8H), 2.61 (s, 7H), 2.10-1.97 (m, 2H), 1.88 (s, 1H), 1.61-1.47 (s, 4H), 1.41-1.29 (m, 12H), 1.24 (t, 3H), 0.99 (d, 3H).

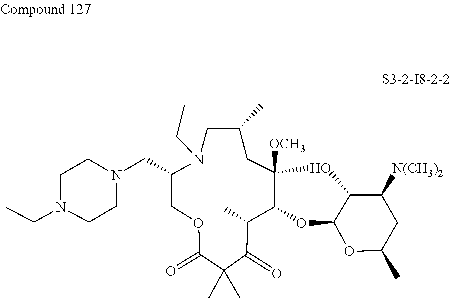 [SMILES: CCN1CCN(C[C@H]2COC(=O)C(C)(C)C(=O)[C@H](C)[C@H]3O[C@@H]4O[C@H](C)C[C@H](N(C)C)[C@H]4O[C@@]3(OC)C[C@@H](C)CN2CC)CC1]

(3S,6R,8R,9R,10R)-9-(((2S,3R,4S,6R)-4-(Dimethylamino)-3-hydroxy-6-methyltetrahydro-2H-pyran-2-yl)oxy)-4-ethyl-3-((4-ethylpiperazin-1-yl)methyl)-8-methoxy-6,8,10,12,12-pentamethyl-1-oxa-4-azacyclotridecane-11,13-dione (S3-2-I8-2-2)

Prepared according to the methods of S3-2-I4-1-2 from S3-2-I8-2 and acetaldehyde to provide 6.83 mg of the title compound as a formate salt. (ESI+) m/z: 214.41 [M+3H]3+, 321.03 [M+2H]2+, 641.14 [M+H]+; 1H NMR (400 MHz, Methanol-d4) δ 8.52 (s, 2H), 4.54 (s, 1H), 4.46 (d, 1H), 4.19 (s, 1H), 4.09 (d, 1H), 3.78-3.65 (m, 1H), 3.59 (s, 1H), 3.44 (dd, 2H), 3.35 (dd, 1H), 2.90 (s, 8H), 2.83 (d, 3H), 2.78 (s, 9H), 2.60 (s, 4H), 2.00 (ddd, 2H), 1.91 (s, 1H), 1.53 (d, 3H), 1.48 (dd, 1H), 1.36 (s, 4H), 1.32 (d, 9H), 1.23 (t, 4H), 1.19-1.13 (m, 2H), 0.94 (d, 3H).

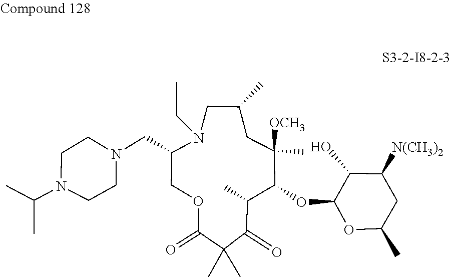 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CN1CCN(C(C)C)CC1]

(3S,6R,8R,9R,10R)-9-(((2S,3R,4S,6R)-4-(Dimethylamino)-3-hydroxy-6-methyltetrahydro-2H-pyran-2-yl)oxy)-3-((4-isopropylpiperazin-1-yl)methyl)-8-methoxy-4,6,8,10,12,12-hexamethyl-1-oxa-4-azacyclotridecane-11,13-dione (S3-2-I8-2-3)

Prepared according to the methods of S3-2-I4-1-2 from S3-2-I8-2 and acetone to provide 6.11 mg of the title compound as a formate salt. (ESI+) m/z: 219.05 [M+3H]3+, 328.02 [M+2H]2+, 655.08 [M+H]+; 1H NMR (400 MHz, Methanol-d4) δ 8.52 (s, 2H), 4.53 (s, 1H), 4.46 (d, 1H), 4.18 (s, 1H), 4.08 (d, 1H), 3.76-3.66 (m, 1H), 3.61 (s, 1H), 3.44 (dd, 1H), 3.35 (dd, 1H), 3.25 (d, 1H), 3.07 (s, 4H), 2.89 (s, 6H), 2.78 (s, 7H), 2.71-2.52 (m, 4H), 2.01 (dd, 2H), 1.88 (s, 1H), 1.52 (s, 3H), 1.51-1.44 (m, 1H), 1.36 (s, 3H), 1.34-1.22 (m, 15H), 1.18 (d, 3H), 0.93 (d, 3H).

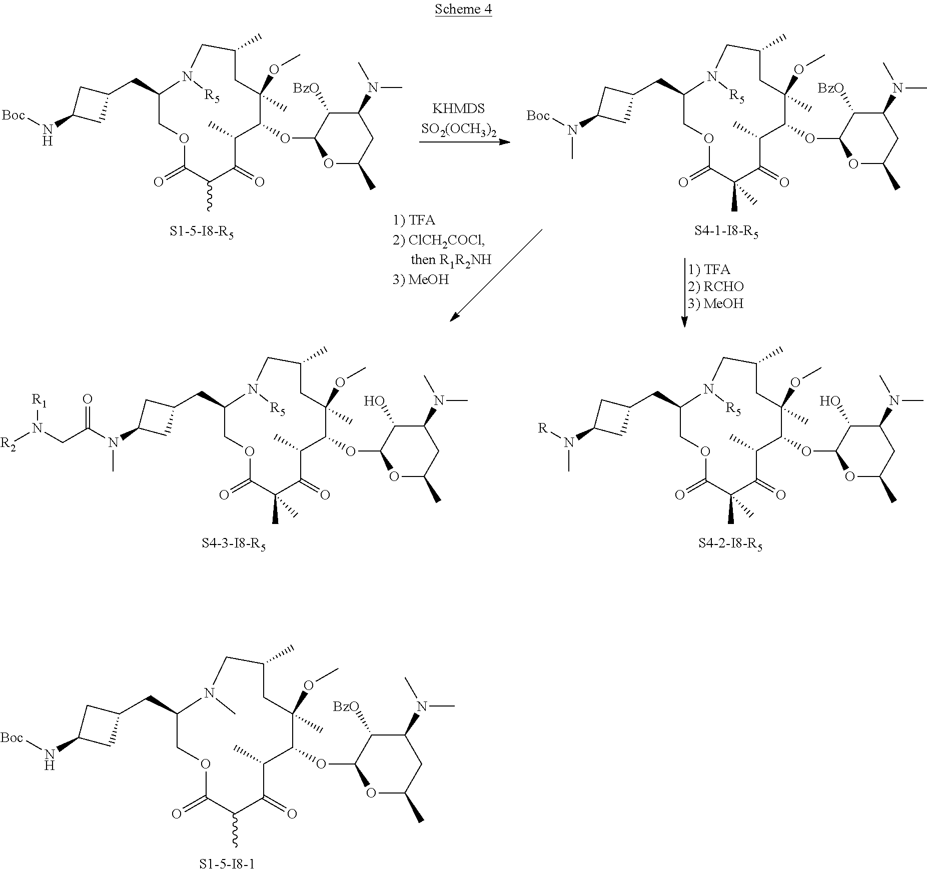

(2S,3R,4S,6R)-2-(((3R,6R,8R,9R,10R)-3-(((1r,3S)-3-((tert-Butoxycarbonyl)amino)cyclobutyl)methyl)-8-methoxy-4,6,8,10,12-pentamethyl-11,13-dioxo-1-oxa-4-azacyclotridecan-9-yl)oxy)-4-(dimethylamino)-6-methyltetrahydro-2H-pyran-3-yl benzoate (S1-5-I8-1)

Prepared according to the methods of S1-5-I1-1, substituting 18, to give 218 mg of the title compound. MS (ESI+) m/z: 387.72 [M+2H]2+, 774.25 [M+H]+; 1H NMR (400 MHz, Chloroform-d) δ 8.10-7.90 (m, 2H), 7.56 (t, 1H), 7.44 (t, 2H), 5.04 (dd, 1H), 4.76-4.61 (m, 1H), 4.55 (d, 1H), 4.35 (s, 1H), 4.23-4.09 (m, 1H), 4.06 (d, 1H), 3.72 (t, 1H), 3.62-3.44 (m, 2H), 3.44-3.19 (m, 1H), 2.91 (d, 1H), 2.87-2.78 (m, 1H), 2.77 (s, 2H), 2.70-2.58 (m, 1H), 2.46 (d, 1H), 2.37-2.29 (m, 1H), 2.26 (s, 4H), 2.16 (s, 2H), 2.09 (s, 1H), 2.07-1.86 (m, 5H), 1.80 (t, 2H), 1.43 (s, 9H), 1.34-1.30 (m, 1H), 1.27 (d, 3H), 1.22 (s, 3H), 1.18 (d, 3H), 0.99 (dd, 4H), 0.83 (dd, 3H).

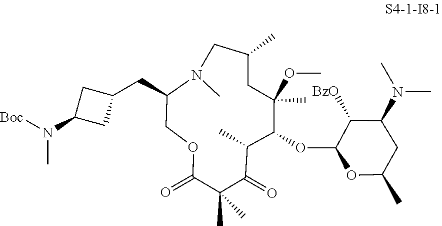

(2S,3R,4S,6R)-2-(((3R,6R,8R,9R,10R)-3-(((1r,3S)-3-((tert-Butoxycarbonyl)(methyl)amino)cyclobutyl)methyl)-8-methoxy-4,6,8,10,12,12-hexamethyl-11,13-dioxo-1-oxa-4-azacyclotridecan-9-yl)oxy)-4-(dimethylamino)-6-methyltetrahydro-2H-pyran-3-yl benzoate (S4-1-I8-1)

S1-5-I8-1 (212 mg, 0.273 mmol) was dissolved in 1,2-dimethoxyethane (2.72 mL), and the reaction mixture was cooled to −78° C. in a dry ice/acetone bath. Potassium bis(trimethylsilyl)amide (1.0 M solution in THF; 0.818 mL, 0.818 mmol) was added. After 5 min, dimethyl sulfate (0.128 mL, 1.36 mmol) was added. The dry ice was removed from the acetone bath, and the reaction mixture was allowed to slowly warm to −10° C. over 50 min. Triethylamine (0.378 mL, 2.27 mmol) was added and the reaction was warmed to room temperature over 30 min. The reaction was quenched by the addition of NH4Cl (sat., aq. solution) and was diluted with EtOAc. The EtOAc layer was washed with water (2 times) and brine (1 time), was dried over Na2SO4, filtered and concentrated. The residue was purified on 12 g of silica gel (elution with 0-12% MeOH-dichloromethane-0.5% NH4OH gradient) to give the 120 mg of the title compound. MS (ESI+) m/z: 401.77 [M+2H]2+, 802.19 [M+H]+; 1H NMR (400 MHz, Chloroform-d) δ 8.08-7.97 (m, 2H), 7.59-7.49 (m, 1H), 7.43 (t, 2H), 5.03 (dd, 1H), 4.60 (d, 2H), 4.04-3.86 (m, 3H), 3.58 (dd, 1H), 3.51-3.36 (m, 1H), 2.82 (d, 7H), 2.50-2.39 (m, 1H), 2.25 (s, 7H), 2.21 (s, 3H), 2.05-1.96 (s, 1H), 1.95-1.79 (m, 4H), 1.80-1.57 (m, 3H), 1.43 (s, 9H), 1.38 (s, 4H), 1.31 (s, 3H), 1.27 (d, 4H), 1.22 (s, 3H), 1.04 (d, 3H), 0.94 (dd, 1H), 0.83 (d, 4H).

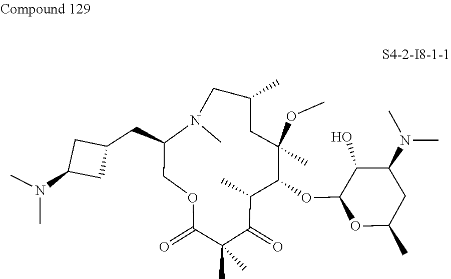

(3R,6R,8R,9R,10R)-9-(((2S,3R,4S,6R)-4-(Dimethylamino)-3-hydroxy-6-methyltetrahydro-2H-pyran-2-yl)oxy)-3-(((1r,3S)-3-(dimethylamino)cyclobutyl)methyl)-8-methoxy-4,6,8,10,12,12-hexamethyl-1-oxa-4-azacyclotridecane-11,13-dione (S4-2-I8-1-1)

A solution of S4-1-I8-1 (120 mg, 0.149 mmol) in dichloromethane (1 mL) and trifluoroacetic acid (0.25 mL) was stirred at room temperature for 2 hr and concentrated. The residue was suspended in ethyl acetate and washed with sat. aq. NaHCO₃(2 times), the washed solution was dried over sodium sulfate, filtered and concentrated in vacuo. The resulting secondary amine (25 mg, 0.0356 mmol) was dissolved in dichloromethane (1 mL), Na(OAc)3BH (15 mg, 0.0712 mmol) followed by formaldehyde (37 wt % aqueous solution, 0.0238 mL, 0.356 mmol) was added. After 15 min, the reaction mixture was quenched with sat. aq. NaHCO₃ and extracted with dichloromethane (3 times). The combined extracts were concentrated in vacuo. The residue was dissolved in methanol (1.5 mL), and the reaction mixture was heated to 45° C. external temperature for 16 hr. Solvent was removed in vacuo and the residue was purified by HPLC (Atlantis T3 column, 2-40% MeCN-water-0.1% HCO2H) to give the title compound as a formate salt (15.8 mg, 0.0236 mmol, 61%). MS (ESI+) m/z: 204.79 [M+3H]3+, 306.59 [M+2H]2+, 612.21 [M+H]+; 1H NMR (400 MHz, Methanol-d4) δ 8.55 (s, 2H), 4.69 (s, 1H), 4.42 (d, 1H), 4.36-4.01 (m, 2H), 3.68 (ddd, 2H), 3.36 (dd, 1H), 3.24-2.69 (m, 9H), 2.60 (s, 7H), 2.40-2.14 (m, 10H), 1.94 (dd, 4H), 1.88-1.69 (m, 1H), 1.52 (s, 3H), 1.47-1.25 (m, 13H), 1.03 (s, 3H).

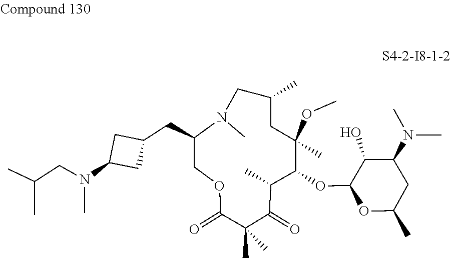

(3R,6R,8R,9R,10R)-9-(((2S,3R,4S,6R)-4-(dimethylamino)-3-hydroxy-6-methyltetrahydro-2H-pyran-2-yl)oxy)-3-(((1r,3S)-3-(isobutyl(methyl)amino)cyclobutyl)methyl)-8-methoxy-4,6,8,10,12,12-hexamethyl-1-oxa-4-azacyclotridecane-11,13-dione (S4-2-I8-1-2)

Prepared according to the methods of S4-2-I8-1-1, substituting isobutyraldehyde to provide 11.09 mg of the title compound as a formate salt. MS (ESI+) m/z: 218.78 [M+3H]3+, 327.61 [M+2H]2+, 654.31 [M+H]+; 1H NMR (400 MHz, Methanol-d4) δ 8.54 (s, 2.5H), 4.66 (s, 1H), 4.44 (d, 1H), 4.33-4.12 (m, 2H), 3.72 (ddd, 1H), 3.66 (s, 0.5H), 3.53 (s, 1H), 3.47-3.34 (m, 2H), 3.34-3.26 (m, 1H), 3.04 (s, 5H), 2.84 (s, 3H), 2.76 (s, 6H), 2.58-2.30 (m, 8H), 2.29-1.94 (m, 6H), 1.87-1.57 (m, 3H), 1.56-1.44 (m, 4H), 1.40 (d, 6H), 1.33 (dd, 6H), 1.05 (d, 3H), 1.01 (d, 6H).

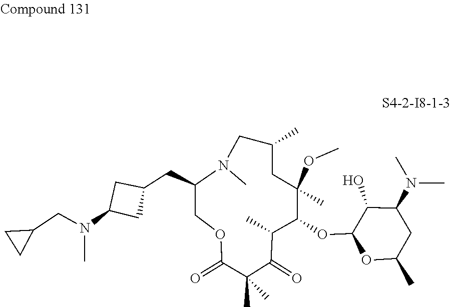

(3R,6R,8R,9R,10R)-3-(((1r,3S)-3-((cyclopropylmethyl)(methyl)amino)cyclobutyl)methyl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S4-2-I8-1-3)

Prepared according to the methods of S4-2-I8-1-1, substituting cyclopropanecarboxaldehyde to provide 16.73 mg of the title compound as a formate salt. MS (ESI+) m/z: 218.12 [M+3H]3+, 326.61 [M+2H]2+, 652.27 [M+H]+; 1H NMR (400 MHz, Methanol-d4) δ 8.55 (s, 2.6H), 4.65 (s, 1H), 4.44 (d, 1H), 4.32-4.13 (m, 2H), 3.78-3.67 (m, 2H), 3.60 (s, 0.4H), 3.48-3.34 (m, 2H), 3.35-3.25 (m, 1H), 3.04 (s, 4H), 2.90-2.77 (m, 4H), 2.76 (s, 7H), 2.69 (s, 3H), 2.59-2.34 (m, 3H), 2.27-1.95 (m, 5H), 1.87-1.58 (m, 3H), 1.56-1.44 (m, 4H), 1.39 (d, 6H), 1.33 (dd, 6H), 1.15-0.96 (m, 4H), 0.76-0.68 (m, 2H), 0.42-0.28 (m, 2H).

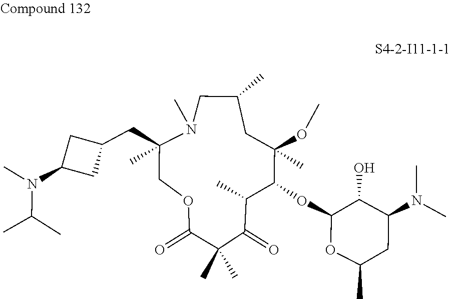

(3R,6R,8R,9R,10R)-9-(((2S,3R,4S,6R)-4-(dimethylamino)-3-hydroxy-6-methyltetrahydro-2H-pyran-2-yl)oxy)-3-(((1r,3S)-3-(isopropyl(methyl)amino)cyclobutyl)methyl)-8-methoxy-3,4,6,8,10,12,12-heptamethyl-1-oxa-4-azacyclotridecane-11,13-dione (S4-2-I11-1-1)

Prepared according to the methods of S4-2-I8-1-1, substituting intermediate I11 and acetone to provide 5.5 mg of the title compound as a formate salt. MS (ESI+) m/z: 218.82 [M+3H]3+, 327.69 [M+2H]2+, 654.32[M+H]+; ¹H NMR (400 MHz, Methanol-d) δ8.55 (s, 3H), 5.10 (d, 1H), 4.46 (d, 1H), 4.19 (d, 1H), 3.89 (d, 1H), 3.82 (m, 1H), 3.75-3.65 (m, 1H), 3.49-3.36 (m, 3H), 3.23-3.12 (m, 2H), 3.08 (s, 3H), 2.90 (s, 3H), 2.82 (d, 1H), 2.69 (s, 6H), 2.59-2.42 (m, 6H), 2.34-2.11 (m, 4H), 2.00-1.88 (m, 2H), 1.83 (d, 1H), 1.51 (s, 4H), 1.45 (d, 1H), 1.41 (s, 3H), 1.38 (t, 6H), 1.31 (d, 4H), 1.28 (q, 4H), 1.22 (dd, 5H), 1.09 (d, 3H).

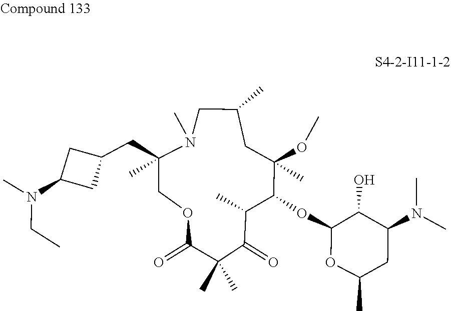

(3R,6R,8R,9R,10R)-9-(((2S,3R,4S,6R)-4-(dimethylamino)-3-hydroxy-6-methyltetrahydro-2H-pyran-2-yl)oxy)-3-(((1r,3S)-3-(ethyl(methyl)amino)cyclobutyl)methyl)-8-methoxy-3,4,6,8,10,12,12-heptamethyl-1-oxa-4-azacyclotridecane-11,13-dione (S4-2-I11-1-2)

Prepared according to the methods of S4-2-I8-1-1, substituting intermediate I11 and acetaldehyde to provide 7.3 mg of the title compound as a formate salt. MS (ESI+) m/z: MS (ESI+) m/z: 214.12 [M+3H]3+, 320.63 [M+2H]², 640.34 [M+H]+; ¹H NMR (400 MHz, Methanol-d) δ 8.54 (s, 3H), 5.08 (d, 1H), 4.47 (d, 1H), 4.20 (d, 1H), 3.89 (d, 1H), 3.75-3.67 (m, 1H), 3.57-3.49 (m, 1H), 3.45-3.46 (m, 2H), 3.29-3.23 (m, 1H), 3.19-3.13 (m, 1H), 3.07 (d, 3H), 2.89 (s, 3H), 2.85-2.78 (m, 3H), 2.73 (s, 6H), 2.52 (s, 3H), 2.50-2.39 (m, 3H), 2.30 (s, 1H), 2.20-2.12 (m, 3H), 1.99 (ddd, 1H), 1.90 (dd, 1H), 1.82 (d, 1H), 1.55-1.44 (m, 5H), 1.41 (s, 3H), 1.38 (t, 6H), 1.32 (d, 3H), 1.28 (s, 3H), 1.24 (t, 3H), 1.08 (d, 3H).

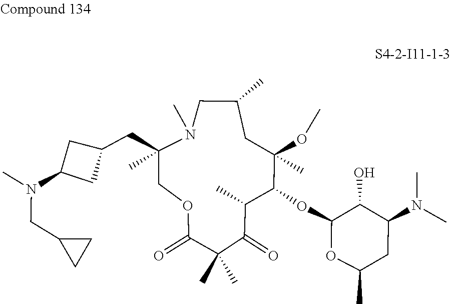

(3R,6R,8R,9R,10R)-3-(((1r,3S)-3-((cyclopropylmethyl)(methyl)amino)cyclobutyl)methyl)-9-(((2S,3R,4S,6R)-4-(dimethylamino)-3-hydroxy-6-methyltetrahydro-2H-pyran-2-yl)oxy)-8-methoxy-3,4,6,8,10,12,12-heptamethyl-1-oxa-4-azacyclotridecane-11,13-dione (S4-2-I11-1-3)

Prepared according to the methods of S4-2-I8-1-1, substituting intermediate I11 and cyclopropanecarbaldehyde to provide 10.24 mg of the title compound as a formate salt. MS (ESI+) m/z: 222.83 [M+3H]3+, 333.67 [M+2H]2+, 666.30 [M+H]+. ¹H NMR (400 MHz, Methanol-d) δ8.54 (s, 3H), 5.08 (d, 1H), 4.47 (d, 1H), 4.20 (d, 1H), 3.89 (d, 1H), 3.76-3.67 (m, 1H), 3.63-3.56 (m, 1H), 3.45-3.38 (m, 2H), 3.30-3.23 (m, 1H), 3.18 (t, 1H), 3.07 (s, 3H), 2.89 (s, 3H), 2.85-2.77 (m, 1H), 2.74 (s, 6H), 2.71 (d, 2H), 2.65 (s, 3H), 2.53-2.42 (m, 3H), 2.39-2.23 (m, 1H), 2.23-2.12 (m, 3H), 2.04-1.96 (m, 1H), 1.93-1.76 (m, 2H), 1.51 (s, 4H), 1.45 (d, 1H), 1.41 (s, 3H), 1.38 (t, 6H), 1.32 (d, 3H), 1.28 (s, 3H), 1.08 (d, 3H), 1.05-0.99 (m, 1H), 0.72-0.66 (m, 2H), 0.37-0.30 (m, 2H).

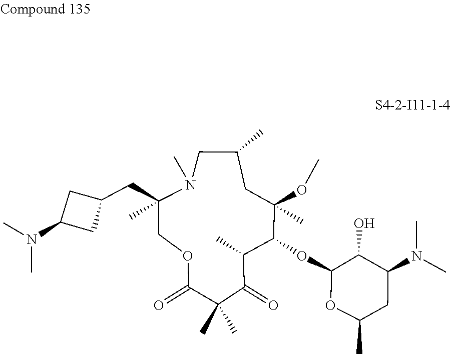

(3R,6R,8R,9R,10R)-9-(((2S,3R,4S,6R)-4-(dimethylamino)-3-hydroxy-6-methyltetrahydro-2H-pyran-2-yl)oxy)-3-(((1r,3S)-3-(dimethylamino)cyclobutyl)methyl)-8-methoxy-3,4,6,8,10,12,12-heptamethyl-1-oxa-4-azacyclotridecane-11,13-dione (S4-2-I11-1-4)

Prepared according to the methods of S4-2-I8-1-1, substituting intermediate I11 and formaldehyde to provide 9.34 mg of the title compound as a formate salt. MS (ESI+) m/z: 209.48 [M+3H]3+, 313.65 [M+2H]2+, 626.27 [M+H]+. ¹H NMR (400 MHz, Methanol-d) δ8.54 (s, 3H), 5.07 (d, 1H), 4.47 (d, 1H), 4.20 (d, 1H), 3.89 (d, 1H), 3.77-3.66 (m, 1H), 3.45-3.36 (m, 2H), 3.30-3.12 (m, 3H), 3.07 (s, 3H), 2.89 (s, 3H), 2.81 (d, 1H), 2.72 (s, 6H), 2.55-2.48 (m, 1H), 2.45 (s, 6H), 2.39-2.27 (m, 3H), 2.19-2.06 (m, 3H), 1.98 (ddd, 1H), 1.92-1.76 (m, 2H), 1.47 (d, 1H), 1.41 (s, 3H), 1.37 (d, 6H), 1.32 (d, 3H), 1.27 (s, 3H), 1.08 (d, 3H).

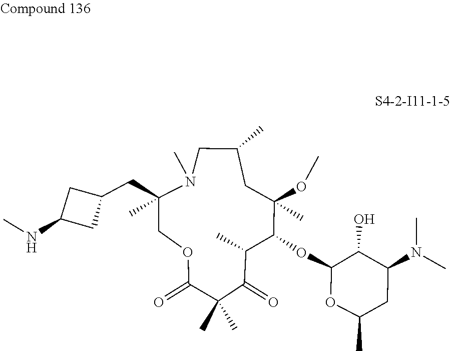

(3R,6R,8R,9R,10R)-9-(((2S,3R,4S,6R)-4-(dimethylamino)-3-hydroxy-6-methyltetrahydro-2H-pyran-2-yl)oxy)-8-methoxy-3,4,6,8,10,12,12-heptamethyl-3-(((1r,3S)-3-(methylamino)cyclobutyl)methyl)-1-oxa-4-azacyclotridecane-11,13-dione (S4-2-I11-1-5)

Prepared according to the methods of S4-2-I8-1-1, substituting intermediate I11 to provide 9.96 mg of the title compound as a formate salt. MS (ESI+) m/z: 204.82 [M+3H]3+, 306.65 [M+2H]2+, 612.22 [M+H]+. ¹H NMR (400 MHz, Methanol-d) δ8.55 (d, 3H), 4.46 (d, 1H), 4.20 (d, 1H), 3.88 (d, 1H), 3.73-3.67 (m, 1H), 3.65-3.58 (m, 1H), 3.40 (dd, 2H), 3.31 (q, 4H), 3.26-3.10 (m, 2H), 3.07 (s, 3H), 2.88 (s, 3H), 2.81 (d, 1H), 2.69 (s, 7H), 2.57 (d, 3H), 2.41-2.20 (m, 5H), 2.19-2.07 (m, 1H), 2.00-1.77 (m, 3H), 1.50 (s, 4H), 1.47-1.43 (m, 1H), 1.40 (s, 3H), 1.38 (s, 4H), 1.36 (s, 2H), 1.33-1.29 (m, 3H), 1.26 (s, 3H), 1.08 (d, 3H).

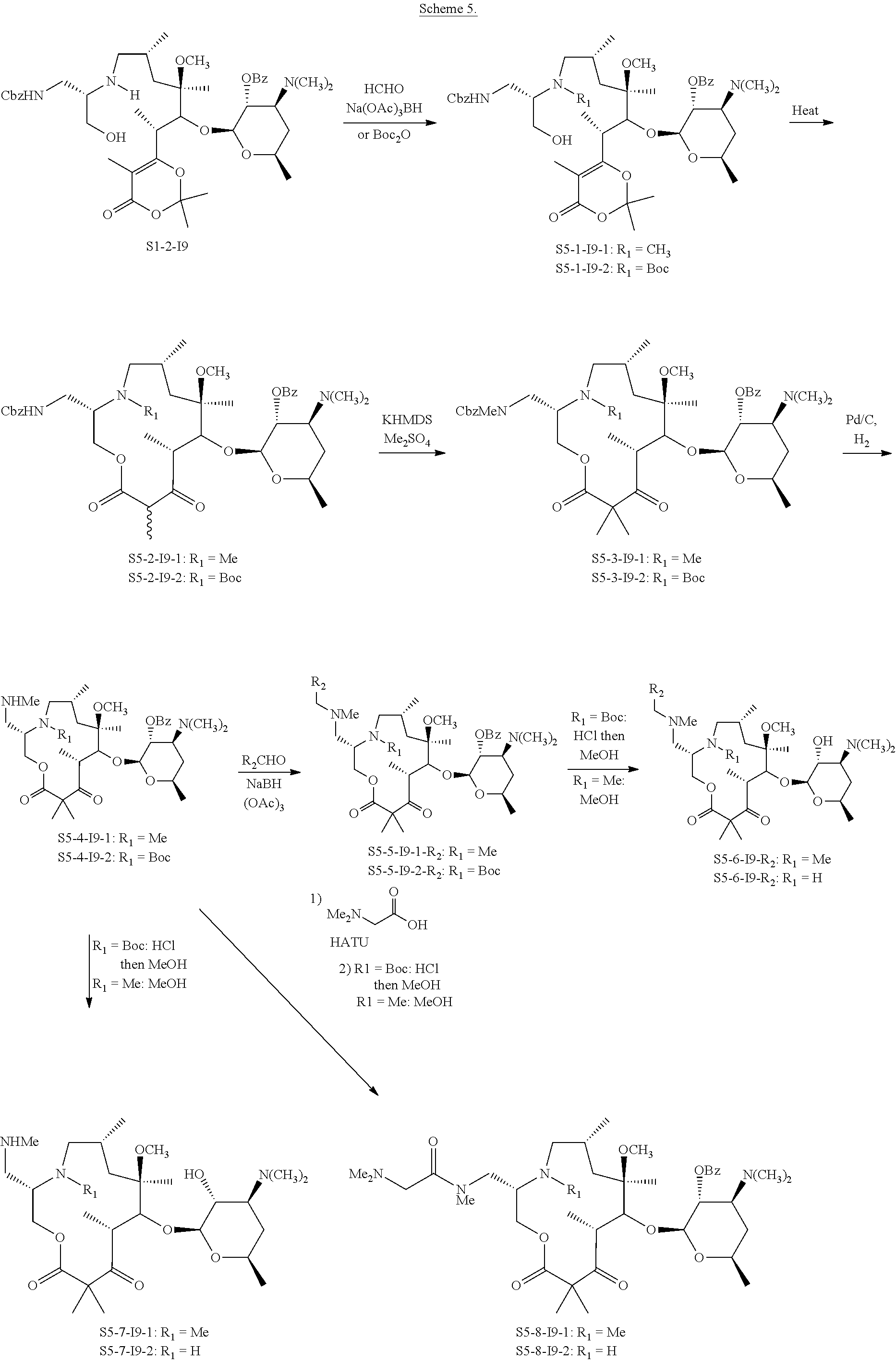

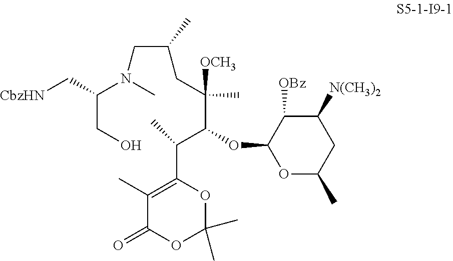

(2S,3R,4S,6R)-2-(((2R,3R,4R,6R)-7-(((S)-1-(((benzyloxy)carbonyl)amino)-3-hydroxypropan-2-yl)(methyl)amino)-4-methoxy-4,6-dimethyl-2-(2,2,5-trimethyl-4-oxo-4H-1,3-dioxin-6-yl)heptan-3-yl)oxy)-4-(dimethylamino)-6-methyltetrahydro-2H-pyran-3-yl benzoate (S5-1-I9-1)

S1-2-19 (380 mg, 0.48 mmol) was dissolved in dry methylene chloride (5 mL) and formaldehyde (0.38 mL, 4.8 mmol) was added. Then NaBH(OAc)3 (201 mg, 0.96 mmol) was added to the reaction mixture In an portion. The reaction was allowed to stir at rt for 10 min and LC/MS shows full conversion. The reaction was quenched by adding saturated NaHCO₃(5 mL) and the aqueous layer was extracted with methylene chloride three times (10 mL). The combined organic layers were dried over MgSO4, filtered and concentrated. The residue was purified on 24 g of silica gel (elution with 0-10% MeOH-dichloromethane+0.5% of 30% aq NH4OH) to give the title compound (310 mg, 80%). MS (ESI+) m/z: 406.8 [M+2H]2+, 812.5 [M+H]+.

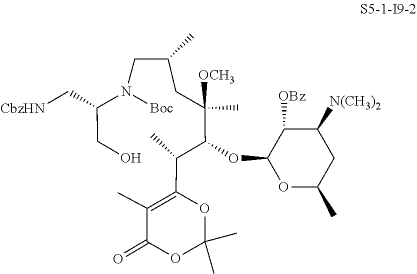

(2S,3R,4S,6R)-2-(((2R,3R,4R,6R)-7-(((S)-1-(((Benzyloxy)carbonyl)amino)-3-hydroxypropan-2-yl)(tert-butoxycarbonyl)amino)-4-methoxy-4,6-dimethyl-2-(2,2,5-trimethyl-4-oxo-4H-1,3-dioxin-6-yl)heptan-3-yl)oxy)-4-(dimethylamino)-6-methyltetrahydro-2H-pyran-3-yl benzoate (S5-1-I9-2)

In a 40 mL vial was a solution of S1-2-19 (410 mg, 0.51 mmol) in dichloromethane (5 mL) to give a yellow solution which was stirred at rt. Boc2O (0.12 mL, 0.51 mmol) was added In an portion and allowed to stir at rt for 2 hours. The reaction was diluted with dichloromethane and poured into satd aq NaHCO₃. The aqueous phase was extracted with dichloromethane and the combined organic phases were dried over MgSO4, filtered and concentrated. The residue was purified on 24 g silica gel (elution with 0-6% MeOH-dichloromethane) to give the title compound (360 mg, 78%). MS (ESI+) m/z: 898.5 [M+H]+.

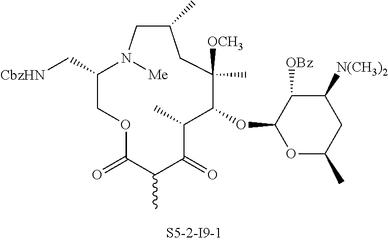

(2S,3R,4S,6R)-2-(((3S,6R,8R,9R,10R)-3-((((benzyloxy)carbonyl)amino)methyl)-8-methoxy-4,6,8,10,12-pentamethyl-11,13-dioxo-1-oxa-4-azacyclotridecan-9-yl)oxy)-4-(dimethylamino)-6-methyltetrahydro-2H-pyran-3-yl benzoate (S5-2-I9-1)

S5-1-I9-1 (310 mg, 0.38 mmol) was concentrated twice from toluene in a 250 mL flask. The flask was fitted with a reflux condenser and the condenser was flame dried under vacuum, allowed to cool and backfilled with nitrogen. Chlorobenzene (95 mL) was added via cannula and the flask was placed under mild vacuum and sonicated for 2 minutes, then backfilled with nitrogen. The degassing procedure was repeated, then the mixture was heated at a bath temperature of 155° C. for 16 hours and then at a bath temperature of 165° C. for 4 hours. The reaction was allowed to cool to rt and was concentrated. The residue was purified on 24 g of silica gel (elution with 0-10% MeOH-dichloromethane+0.5% of 30% aq NH4OH) to give the title compound (242 mg, 82%). MS (ESI+) m/z: 377.7 [M+2H]2+, 754.4 [M+H]+.

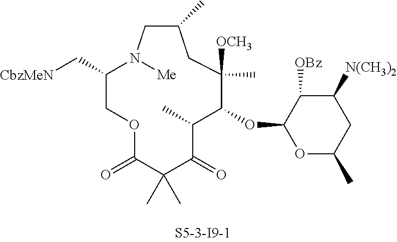

(2S,3R,4S,6R)-2-(((3S,6R,8R,9R,10R)-3-((((Benzyloxy)carbonyl)(methyl)amino)methyl)-8-methoxy-4,6,8,10,12,12-hexamethyl-11,13-dioxo-1-oxa-4-azacyclotridecan-9-yl)oxy)-4-(dimethylamino)-6-methyltetrahydro-2H-pyran-3-yl benzoate (S5-3-I9-1)

In a 20 mL vial was a solution of S5-2-I9-1 (242 mg, 0.32 mmol) in 1,2-dimethoxyethane (5 mL) precooled at −60° C. KHMDS (0.96 mL, 0.96 mmol) was added dropwise. The reaction mixture was stirred at −60° C. for 20 min. Then Me2SO4 (150 μL, 1.59 mmol) was added. The reaction mixture was allowed to warm to −15° C. LC/MS shows full conversion. The reaction was quenched by adding triethylamine (1 mL) and the resulting mixture was diluted with dichloromethane and saturated NaHCO₃ was added. The aqueous layer was extracted with dichloromethane and the combined organic layers were dried over MgSO4, filtered and concentrated. The residue was purified on 4 g of silica gel (elution with 0-10% MeOH-dichloromethane+0.5% of 30% aq NH4OH) to give the title compound (220 mg, 88%). MS (ESI+) m/z: 391.8 [M+2H]2+, 782.5 [M+H]+.

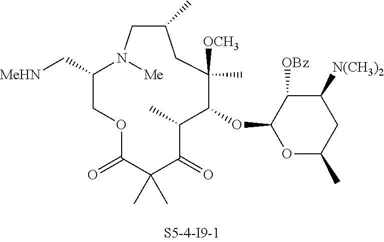

(2S,3R,4S,6R)-4-(Dimethylamino)-2-(((3S,6R,8R,9R,10R)-8-methoxy-4,6,8,10,12,12-hexamethyl-3-((methylamino)methyl)-11,13-dioxo-1-oxa-4-azacyclotridecan-9-yl)oxy)-6-methyltetrahydro-2H-pyran-3-yl benzoate (S5-4-I9-1)

S5-3-I9-1 (220 mg, 0.28 mmol) was dissolved in EtOAc (5 mL) and AcOH (32 μL, 0.56 mmol) was added. The reaction mixture was sonicated briefly under mild vacuum, then backfilled with nitrogen. Pd/C (60 mg, 0.028 mmol) was added and the mixture was stirred under streaming hydrogen for 10 minutes, then under static hydrogen until LC/MS indicated complete consumption of starting material. The reaction mixture was filtered through a syringe filter with the aid of EtOAc, and saturated NaHCO₃(5 mL) was added. The aqueous layer was extracted with methylene chloride three times (10 mL). The combined organic layers were dried over MgSO4, filtered and concentrated. The crude title compound (154 mg, 85%) was used in the next step without further purification. MS (ESI+) m/z: 216.8 [M+3H]3+, 324.7 [M+2H]2+, 648.4 [M+H]+.

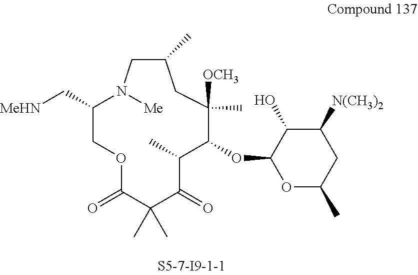 [SMILES: CNC[C@H]1COC(=O)C(C)(C)C(=O)[C@H](C)[C@@H](O[C@@H]2O[C@H](C)C[C@H](N(C)C)[C@H]2O)[C@](C)(OC)C[C@@H](C)CN1C]

(3S,6R,8R,9R,10R)-9-(((2S,3R,4S,6R)-4-(Dimethylamino)-3-hydroxy-6-methyltetrahydro-2H-pyran-2-yl)oxy)-8-methoxy-4,6,8,10,12,12-hexamethyl-3-((methylamino)methyl)-1-oxa-4-azacyclotridecane-11,13-dione (S5-7-I9-1-1)

S5-4-I9-1 (39 mg, 0.06 mmol) was dissolved in MeOH (0.5 mL) and heated at 60° C. until LC/MS indicated complete consumption of starting material (16 hours). The reaction mixture was filtered through a syringe filter with the aid of methanol and concentrated. The residue was purified by HPLC (MeCN-water-0.1% HCO2H) to yield 9.07 mg of the title compound as a formate salt. MS (ESI+) m/z: 182.1 [M+3H]3+, 272.7 [M+2H]2+, 544.4 [M+H]+; 1H NMR (400 MHz, Methanol-d4) δ 8.50 (s, 3H), 4.45 (dd, 1H), 4.23 (dd, 1H), 4.10 (d, 1H), 3.77-3.65 (m, 1H), 3.55-3.27 (m, 4H), 3.17-3.03 (m, 1H), 2.90 (d, 3H), 2.81 (d, 7H), 2.69 (d, 1H), 2.65 (s, 2H), 2.33 (s, 1H), 2.06-1.97 (m, 1H), 1.53 (s, 3H), 1.51-1.43 (m, 1H), 1.37-1.19 (m, 12H), 0.97 (dd, 3H).

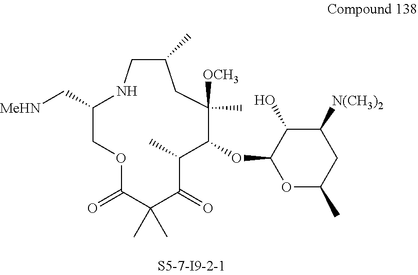 [SMILES: CNC[C@H]1COC(=O)C(C)(C)C(=O)[C@H](C)[C@@H](O[C@@H]2O[C@H](C)C[C@H](N(C)C)[C@H]2O)[C@](C)(OC)C[C@@H](C)CN1]

(3S,6R,8R,9R,10R)-9-(((2S,3R,4S,6R)-4-(Dimethylamino)-3-hydroxy-6-methyltetrahydro-2H-pyran-2-yl)oxy)-8-methoxy-6,8,10,12,12-pentamethyl-3-((methylamino)methyl)-1-oxa-4-azacyclotridecane-11,13-dione (S5-7-I9-2-1)

S5-4-I9-2 (39 mg, 0.06 mmol) was dissolved in MeOH (0.5 mL) and heated at 60° C. until LC/MS indicated complete consumption of starting material (16 hours). The reaction mixture was cooled, and aqueous HCl (4 M, 52 μL, 4 equiv) was added. The reaction mixture was allowed to stir at rt until LC/MS indicated complete consumption of starting material. The reaction mixture was filtered through a syringe filter with the aid of methanol and concentrated. The residue was purified by HPLC (MeCN-water-0.1% HCO2H) to yield 2.35 mg of the title compound as a formate salt. MS (ESI+) m/z: 177.5 [M+3H]3+, 265.7 [M+2H]2+, 530.4 [M+H]+; 1H NMR (400 MHz, Methanol-d4) δ 8.50 (s, 3H), 4.57 (dd, 1H), 4.47 (d, 1H), 4.07-3.95 (m, 2H), 3.82-3.67 (m, 2H), 3.50-3.37 (m, 2H), 3.32 (h, 3H), 3.21 (d, 1H), 3.10-2.86 (m, 3H), 2.86-2.78 (m, 8H), 2.73-2.52 (m, 5H), 2.02 (dt, 1H), 1.92 (s, 1H), 1.63 (dd, 1H), 1.49 (ddd, 4H), 1.37-1.18 (m, 12H), 1.01 (dd, 3H).

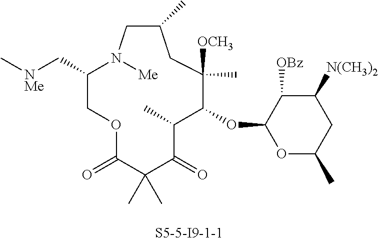

(2S,3R,4S,6R)-4-(Dimethylamino)-2-(((3S,6R,8R,9R,10R)-3-((dimethylamino)methyl)-8-methoxy-4,6,8,10,12,12-hexamethyl-11,13-dioxo-1-oxa-4-azacyclotridecan-9-yl)oxy)-6-methyltetrahydro-2H-pyran-3-yl benzoate (S5-5-I9-1-1)

S5-4-I9-1 (37 mg, 0.057 mmol) was dissolved in dry methylene chloride (1 mL) and formaldehyde (0.046 mL, 0.57 mmol) was added. Then NaBH(OAc)3 (24 mg, 0.12 mmol) was added to the reaction mixture In an portion. The reaction was allowed to stir at rt for 10 min and LC/MS shows full conversion. The reaction was quenched by adding saturated NaHCO₃(5 mL) and the aqueous layer was extracted with methylene chloride three times (10 mL). The combined organic layers were dried over MgSO4, filtered and concentrated. The residue was purified on 4 g of silica gel (elution with 0-10% MeOH-dichloromethane+0.5% of 30% aq NH4OH) to give 37 mg of the title compound. MS (ESI+) m/z: 221.5 [M+3H]3+, 331.7 [M+2H]2+, 662.4 [M+H]+.

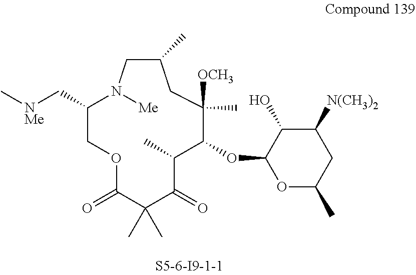 [SMILES: CO[C@]1(C)C[C@@H](C)CN(C)[C@@H](CN(C)C)COC(=O)C(C)(C)C(=O)[C@H](C)[C@H]1O[C@@H]1O[C@H](C)C[C@H](N(C)C)[C@H]1O]

(3S,6R,8R,9R,10R)-9-(((2S,3R,4S,6R)-4-(dimethylamino)-3-hydroxy-6-methyltetrahydro-2H-pyran-2-yl)oxy)-3-((dimethylamino)methyl)-8-methoxy-4,6,8,10,12,12-hexamethyl-1-oxa-4-azacyclotridecane-11,13-dione (S5-6-I9-1-1)

S5-5-I9-1-1 (37 mg, 0.06 mmol) was dissolved in MeOH (0.5 mL) and heated at 60° C. until LC/MS indicated complete consumption of starting material (16 hours). The reaction mixture was filtered through a syringe filter with the aid of methanol and concentrated. The residue was purified by HPLC (MeCN-water-0.1% HCO2H) to yield 9.07 mg of the title compound as a formate salt. MS (ESI+) m/z: 186.8 [M+3H]3+, 279.7 [M+2H]2+, 558.4 [M+H]+; 1H NMR (400 MHz, Methanol-d4) δ 8.55 (s, 3H), 4.42 (d, 2H), 4.14 (d, 3H), 3.68 (dtt, 2H), 3.49 (t, 1H), 3.44-3.28 (m, 4H), 3.13 (s, 2H), 3.02 (s, 1H), 2.80 (s, 1H), 2.67 (d, 11H), 2.44 (dd, 3H), 2.33 (d, 11H), 1.94 (ddd, 2H), 1.44 (t, 5H), 1.38 (s, 6H), 1.36-1.27 (m, 12H), 1.23 (d, 2H), 1.03 (s, 2H), 0.95 (d, 1H).

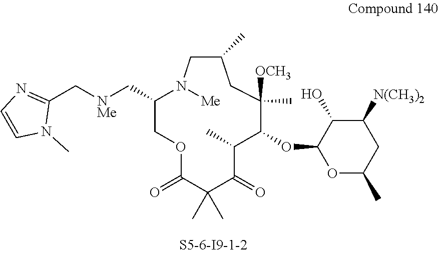 [SMILES: CO[C@]1(C)C[C@@H](C)CN(C)[C@@H](CN(C)Cc2nccn2C)COC(=O)C(C)(C)C(=O)[C@H](C)[C@H]1O[C@@H]1O[C@H](C)C[C@H](N(C)C)[C@H]1O]

(3S,6R,8R,9R,10R)-9-(((2S,3R,4S,6R)-4-(Dimethylamino)-3-hydroxy-6-methyltetrahydro-2H-pyran-2-yl)oxy)-8-methoxy-4,6,8,10,12,12-hexamethyl-3-((methyl((1-methyl-1H-imidazol-2-yl)methyl)amino)methyl)-1-oxa-4-azacyclotridecane-11,13-dione (S5-6-I9-1-2)

Prepared according to the methods of S5-6-I9-1-1, substituting 1-methyl-1H-imidazole-2-carbaldehyde to provide 14.35 mg of the title compound as a formate salt. MS (ESI+) m/z: 240.5 [M+3H]3+, 360.3 [M+2H]2+, 719.5 [M+H]+; 1H NMR (400 MHz, Methanol-d4) δ 8.45 (s, 3H), 7.12 (d, 1H), 6.93 (d, 1H), 4.43 (d, 1H), 4.20 (d, 1H), 4.11 (d, 1H), 3.78-3.66 (m, 5H), 3.66-3.56 (m, 1H), 3.49-3.34 (m, 3H), 3.30 (q, 1H), 3.04 (d, 4H), 2.80 (d, 7H), 2.50 (dt, 1H), 2.39 (s, 3H), 2.07-1.97 (m, 1H), 1.58-1.41 (m, 5H), 1.41-1.21 (m, 13H), 0.98 (d, 3H).

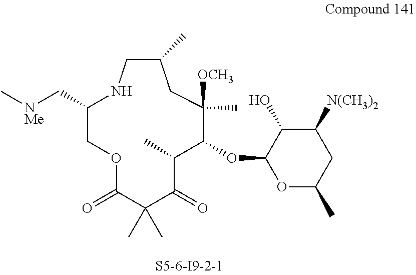 [SMILES: CO[C@]1(C)C[C@@H](C)CN[C@@H](CN(C)C)COC(=O)C(C)(C)C(=O)[C@H](C)[C@H]1O[C@@H]1O[C@H](C)C[C@H](N(C)C)[C@H]1O]

(3S,6R,8R,9R,10R)-9-(((2S,3R,4S,6R)-4-(dimethylamino)-3-hydroxy-6-methyltetrahydro-2H-pyran-2-yl)oxy)-3-((dimethylamino)methyl)-8-methoxy-6,8,10,12,12-pentamethyl-1-oxa-4-azacyclotridecane-11,13-dione (S5-6-I9-2-1)

Prepared by the methods of S5-5-I9-1-1 from S5-5-I9-2-1, (39 mg, 0.06 mmol) was dissolved in MeOH (0.5 mL) and heated at 60° C. until LC/MS indicated complete consumption of starting material (16 hours). The reaction mixture was cooled, and aqueous HCl (4 M, 52 μL, 4 equiv) was added. The reaction mixture was allowed to stir at rt until LC/MS indicated complete consumption of starting material. The reaction mixture was filtered through a syringe filter with the aid of methanol and concentrated. The residue was purified by HPLC (MeCN-water-0.1% HCO2H) to yield 2.33 mg of the title compound as a formate salt. MS (ESI+) m/z: 182.1 [M+3H]3+, 272.7 [M+2H]2+, 544.4 [M+H]+; 1H NMR (400 MHz, Methanol-d4) δ 8.52 (s, 3H), 4.47 (d, 1H), 4.11 (d, 1H), 3.97 (dd, 1H), 3.77-3.61 (m, 3H), 3.44 (dd, 1H), 3.31 (dt, 4H), 2.94 (s, 3H), 2.83 (d, 2H), 2.77 (s, 6H), 2.70-2.58 (m, 2H), 2.58-2.46 (m, 1H), 2.46-2.38 (m, 2H), 2.37 (s, 4H), 2.03 (d, 2H), 1.72 (dd, 1H), 1.63 (dd, 1H), 1.50 (d, 3H), 1.34 (dd, 13H), 1.06 (d, 3H).

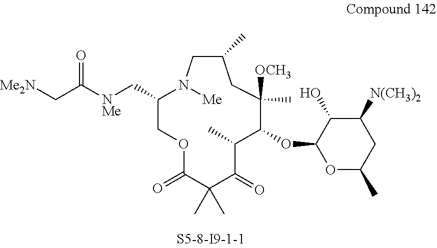 [SMILES: CO[C@]1(C)C[C@@H](C)CN(C)[C@@H](CN(C)C(=O)CN(C)C)COC(=O)C(C)(C)C(=O)[C@H](C)[C@H]1O[C@@H]1O[C@H](C)C[C@H](N(C)C)[C@H]1O]

2-(dimethylamino)-N-(((3S,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-11,13-dioxo-1-oxa-4-azacyclotridecan-3-yl)methyl)-N-methylacetamide (S5-8-I9-1-1)

S5-4-I9-1 (43 mg, 0.066 mmol) was dissolved in DMF (0.5 mL). DIEA (34 μL, 0.20 mmol), dimethylglycine (10.2 mg, 0.10 mmol) and HATU (33 mg, 0.086 mmol) were added at rt. The reaction mixture was allowed to stirred at rt for 2 h. LC/MS indicated complete consumption of starting material. The reaction mixture was diluted with dichloromethane and quenched by adding aqueous NaHCO₃(10 mL). The aqueous layer was extracted with dichloromethane and the combined organic layers were dried over MgSO4, filtered and concentrated. The residue was purified on 4 g of silica gel (elution with 0-10% MeOH-dichloromethane+0.5% of 30% aq NH4OH) to give the crude product (39 mg, 80%). MS (ESI+) m/z: 245.2 [M+3H]3+, 367.3 [M+2H]2+, 733.5 [M+H]+. The material (39 mg, 0.06 mmol) was dissolved in MeOH (0.5 mL) and heated at 60° C. until LC/MS indicated complete consumption of starting material (16 hours). The reaction mixture was filtered through a syringe filter with the aid of methanol and concentrated. The residue was purified by HPLC (MeCN-water-0.1% HCO2H) to yield 5.49 mg of the title compound as a formate salt. MS (ESI+) m/z: 210.3 [M+3H]3+, 314.7 [M+2H]2+, 628.4 [M+H]+; 1H NMR (400 MHz, Methanol-d4) δ 8.27 (s, 3H), 4.57 (dd, 1H), 4.36 (d, 1H), 4.23-4.01 (m, 4H), 3.96-3.83 (m, 3H), 3.60 (q, 2H), 3.49 (dd, 2H), 3.32 (p, 2H), 3.17 (dd, 2H), 3.01 (d, 3H), 2.94 (s, 4H), 2.85 (d, 8H), 2.75 (d, 1H), 2.71 (s, 2H), 2.62 (ddd, 1H), 2.11 (d, 1H), 1.79 (d, 1H), 1.69 (s, 3H), 1.59 (s, 1H), 1.41-1.16 (m, 12H), 0.99 (qd, 7H), 0.84-0.74 (m, 2H), 0.69 (dd, 2H).

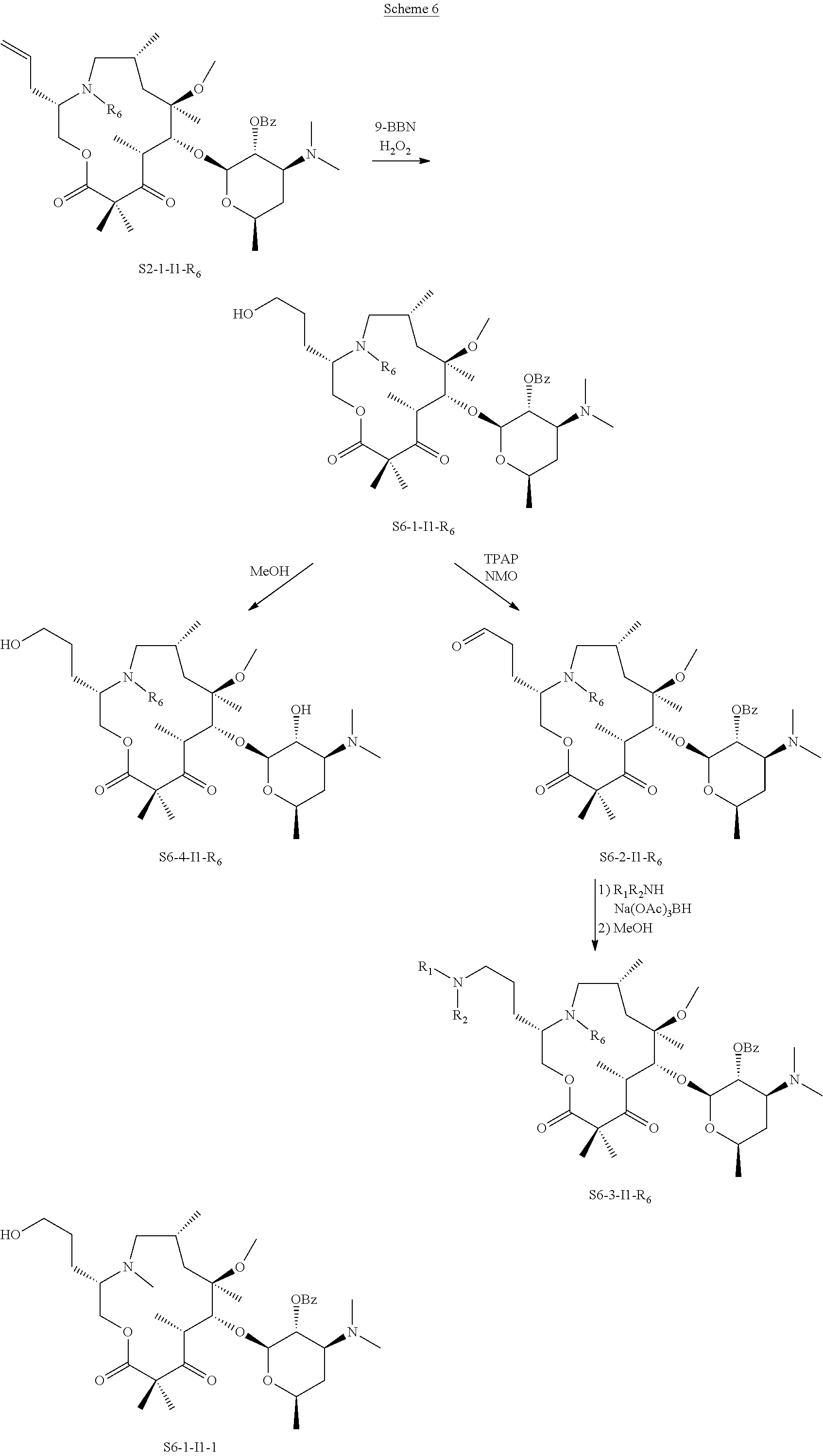

(2S,3R,4S,6R)-4-(dimethylamino)-2-(((3S,6R,8R,9R,10R)-3-(3-hydroxypropyl)-8-methoxy-4,6,8,10,12,12-hexamethyl-11,13-dioxo-1-oxa-4-azacyclotridecan-9-yl)oxy)-6-methyltetrahydro-2H-pyran-3-yl benzoate (S6-1-I1-1)

To S2-1-I1-1 (240 mg, 0.372 mmol) in dry THE (3.71 mL) was added 9-BBN (0.5M solution in THF, 2.22 mL, 1.11 mmol). After 30 min at rt, the mixture was cooled to 0° C. and NaOH (6 N aqueous solution, 371 μL, 2.23 mmol) and H₂O₂ (30% aqueous solution, 252 μL, 2.23 mmol) were added. After 15 min, the mixture was extracted with t-butylmethylether/EtOAc (2:1) three times. The organic layer was washed with water (1 time) and brine (1 time) and was dried over Na2SO4. After the solvent was removed, the residue was purified on 4 g of silica gel (elution with 0-20% MeOH— dichloromethane/0.5% NH4OH gradient) to give the title compound (145 mg, 59%). MS (ESI+) m/z: 663.37 [M+H]+; 1H NMR (400 MHz, Chloroform-d) δ 8.08-7.94 (m, 2H), 7.55 (dd, 1H), 7.44 (t, 2H), 5.03 (dd, 1H), 4.57 (d, 1H), 4.10 (dd, 1H), 4.01 (d, 1H), 3.95 (dd, 1H), 3.72-3.50 (m, 3H), 3.41 (dt, 1H), 3.04 (s, 1H), 2.87-2.81 (m, 1H), 2.80 (s, 3H), 2.32 (dd, 1H), 2.26 (s, 6H), 2.10 (t, 1H), 1.93 (d, 1H), 1.83-1.47 (m, 10H), 1.40 (s, 4H), 1.31-1.22 (m, 9H), 1.16-1.07 (m, 1H), 1.03 (d, 3H), 0.91 (d, 3H).

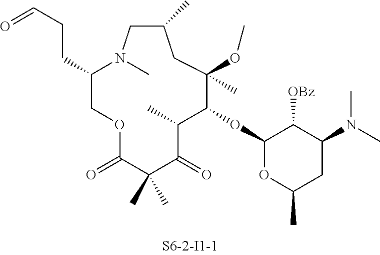

(2S,3R,4S,6R)-4-(dimethylamino)-2-(((3S,6R,8R,9R,10R)-8-methoxy-4,6,8,10,12,12-hexamethyl-11,13-dioxo-3-(3-oxopropyl)-1-oxa-4-azacyclotridecan-9-yl)oxy)-6-methyltetrahydro-2H-pyran-3-yl benzoate (S6-2-I1-1)

To S6-1-I1-1 (145 mg, 218 mmol) in dry dichloromethane/CH3CN (9:1, 2.9 mL) was added activated 4 A molecular sieves (100 mg, powdered), N-methylmorpholine N-oxide (33 mg, 283 mmol), and tetrapropylammonium perruthenate (4 mg, 10.9 mmol). After 1 h at RT, the solvent was removed. The dried residue was dissolved in t-butylmethylether/Hexane (1:1) and was filtered through Celite® (3 times). After the solvent was removed, the residue was dried under vacuum to give the aldehyde as a white foam. MS (ESI+) m/z: 661.35 [M+H]+. Used directly in the next step.

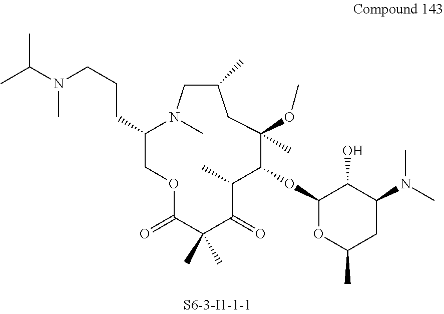

(3S,6R,8R,9R,10R)-9-(((2S,3R,4S,6R)-4-(dimethylamino)-3-hydroxy-6-methyltetrahydro-2H-pyran-2-yl)oxy)-3-(3-(isopropyl(methyl)amino)propyl)-8-methoxy-4,6,8,10,12,12-hexamethyl-1-oxa-4-azacyclotridecane-11,13-dione (S6-3-I1-1-1)

A mixture of S6-2-I1-1 (25 mg, 37.8 mmol) and methylisopropyamine (8 mg, 113 mmol) in dichloromethane (2 mL) was stirred for 30 min, then NaBH(OAc)₃ (12 mg, 56.7 mmol) was added. After 20 min, the solvent was removed, and the residue was dissolved in MeOH (2 mL) and was heated at 50° C. overnight. The reaction was allowed to cool to rt and was concentrated. The residue was purified by HPLC (Atlantis T3 column, 5-50% MeCN-water-0.1% HCO2H) to give 8.6 mg of the title compound as a formate salt. MS (ESI+) m/z: 614.48 [M+H]+; 1H NMR (400 MHz, Methanol-d4) δ 4.45 (d, 1H), 4.28 (d, 1H), 4.20 (d, 1H), 3.88-3.65 (m, 2H), 3.56 (hept, 1H), 3.51-3.23 (m, 4H), 3.05 (t, 7H), 2.79 (s, 8H), 2.72 (s, 3H), 2.17 (s, 1H), 2.02 (ddd, 1H), 1.84 (d, 4H), 1.67-1.41 (m, 7H), 1.45-1.19 (m, 19H), 1.05 (d, 3H).

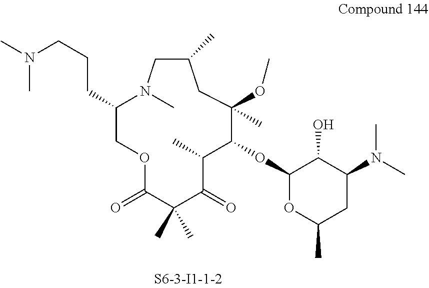

(3S,6R,8R,9R,10R)-9-(((2S,3R,4S,6R)-4-(dimethylamino)-3-hydroxy-6-methyltetrahydro-2H-pyran-2-yl)oxy)-3-(3-(dimethylamino)propyl)-8-methoxy-4,6,8,10,12,12-hexamethyl-1-oxa-4-azacyclotridecane-11,13-dione (S6-3-I1-1-2)

Prepared according to the methods of S6-3-I1-1-1 from dimethylamine to give the title compound as a formate salt. MS (ESI+) m/z: 586.35 [M+H]+; 1H NMR (400 MHz, Methanol-d4) δ 4.44 (d, 1H), 4.27 (s, 3H), 3.71 (q, 2H), 3.41 (t, 2H), 3.32 (p, 6H), 3.22 (s, 1H), 3.06 (s, 4H), 2.70 (s, 10H), 2.47 (s, 5H), 2.07-1.89 (m, 2H), 1.69 (s, 4H), 1.60-1.21 (m, 16H), 1.06 (s, 3H).

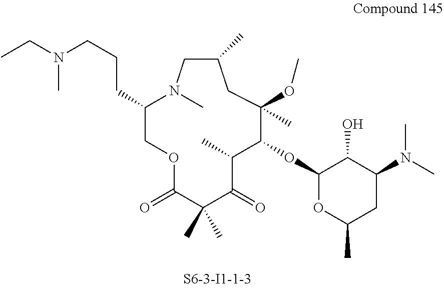

(3S,6R,8R,9R,10R)-9-(((2S,3R,4S,6R)-4-(dimethylamino)-3-hydroxy-6-methyltetrahydro-2H-pyran-2-yl)oxy)-3-(3-(ethyl(methyl)amino)propyl)-8-methoxy-4,6,8,10,12,12-hexamethyl-1-oxa-4-azacyclotridecane-11,13-dione (S6-3-I1-1-3)

Prepared according to the methods of S6-3-I1-1-1 from ethylmethylamine to give the title compound as a formate salt. MS (ESI+) m/z: 600.41 [M+H]+; 1H NMR (400 MHz, Methanol-d4) δ 4.45 (d, 1H), 4.40-4.15 (m, 2H), 3.82-3.61 (m, 2H), 3.53-3.25 (m, 4H), 3.04 (q, 10H), 2.88-2.61 (m, 12H), 2.18 (d, 1H), 2.06-1.97 (m, 1H), 1.86 (t, 4H), 1.62-1.44 (m, 5H), 1.45-1.23 (m, 16H), 1.10-0.95 (m, 3H).

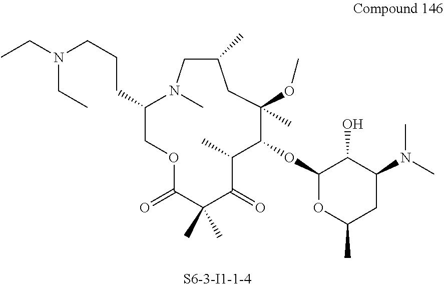

(3S,6R,8R,9R,10R)-3-(3-(diethylamino)propyl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S6-3-I1-1-4)

Prepared according to the methods of S6-3-I1-1-1 from ethylmethylamine to give the title compound as a formate salt. MS (ESI+) m/z: 614.46 [M+H]+; 1H NMR (400 MHz, Methanol-d4) δ 4.45 (d, 1H), 4.38-4.08 (m, 2H), 3.78-3.66 (m, 2H), 3.61 (q, 1H), 3.44 (dd, 2H), 3.37-3.25 (m, 2H), 3.24-2.86 (m, 11H), 2.76 (s, 8H), 2.35-2.09 (m, 1H), 2.04-1.96 (m, 1H), 1.76 (s, 4H), 1.50 (d, 5H), 1.44-1.22 (m, 17H), 1.16 (dt, 3H), 1.03 (s, 3H).

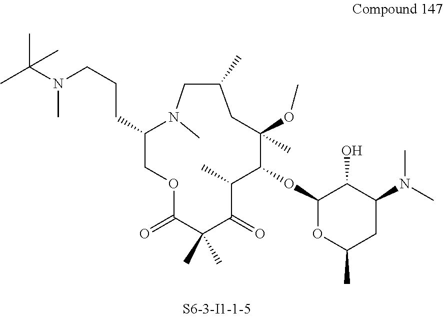

(3S,6R,8R,9R,10R)-3-(3-(tert-butyl(methyl)amino)propyl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S6-3-I1-1-5)

Prepared according to the methods of S6-3-I1-1-1 from t-butylmethylamine to give the title compound as a formate salt. MS (ESI+) m/z: 628.50 [M+H]+; 1H NMR (400 MHz, Methanol-d4) δ 4.44 (d, 1H), 4.38-4.02 (m, 2H), 3.72 (dt, 2H), 3.43 (dd, 2H), 3.38-3.25 (m, 6H), 3.21-2.86 (m, 7H), 2.77 (d, 10H), 2.08-1.95 (m, 2H), 1.87 (s, 4H), 1.51 (s, 5H), 1.46-1.20 (m, 21H), 1.03 (s, 3H).

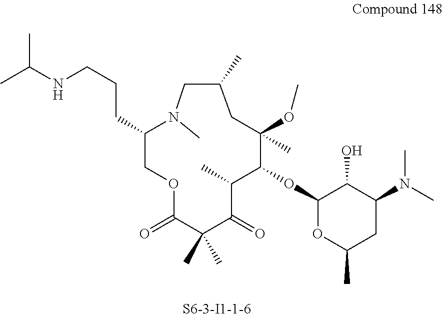

(3S,6R,8R,9R,10R)-9-(((2S,3R,4S,6R)-4-(dimethylamino)-3-hydroxy-6-methyltetrahydro-2H-pyran-2-yl)oxy)-3-(3-(isopropylamino)propyl)-8-methoxy-4,6,8,10,12,12-hexamethyl-1-oxa-4-azacyclotridecane-11,13-dione (S6-3-I1-1-6)

Prepared according to the methods of S6-3-I1-1-1 from isopropylamine to give the title compound as a formate salt. MS (ESI+) m/z: 600.38 [M+H]+; 1H NMR (400 MHz, Methanol-d4) δ 4.49 (d, 1H), 4.27 (s, 1H), 4.15 (d, 1H), 3.72 (ddt, 1H), 3.61-3.34 (m, 5H), 3.31 (dt, 1H), 3.27-3.09 (m, 1H), 2.95 (s, 4H), 2.82 (d, 13H), 2.59 (d, 3H), 2.41-2.12 (m, 2H), 2.10-1.91 (m, 3H), 1.91-1.66 (m, 3H), 1.64-1.45 (m, 5H), 1.43-1.19 (m, 14H), 0.94 (d, 3H).

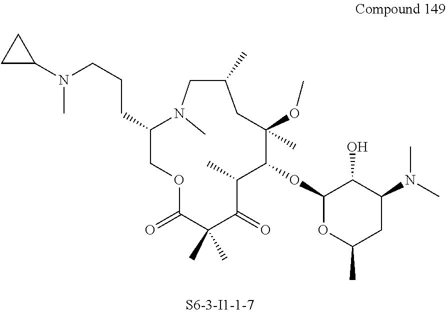

(3S,6R,8R,9R,10R)-3-(3-(cyclopropyl(methyl)amino)propyl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S6-3-I1-1-7)

Prepared according to the methods of S6-3-I1-1-1 from N-methylcyclopropylamine to give the title compound as a formate salt. MS (ESI+) m/z: 612.25 [M+H]+; 1H NMR (400 MHz, Methanol-d4) δ 4.41 (d, 1H), 4.24 (s, 2H), 3.68 (tt, 2H), 3.49-3.26 (m, 2H), 3.04 (s, 6H), 2.79 (s, 1H), 2.61 (d, 9H), 2.37 (d, 4H), 2.20 (s, 1H), 1.91 (d, 3H), 1.71 (tt, 3H), 1.66-1.18 (m, 20H), 1.06 (s, 3H), 0.55 (h, 2H), 0.44 (q, 2H).

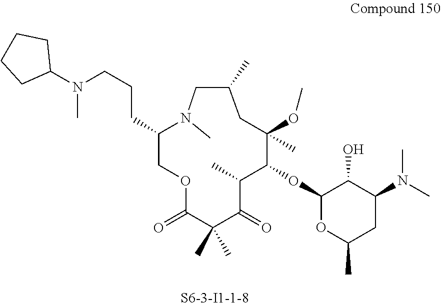

(3S,6R,8R,9R,10R)-3-(3-(cyclopentyl(methyl)amino)propyl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S6-3-I1-1-8)

Prepared according to the methods of S6-3-I1-1-1 from N-methylcyclopentylamine to give the title compound as a formate salt. MS (ESI+) m/z: 640.30 [M+H]+; 1H NMR (400 MHz, Methanol-d4) δ 4.46 (d, 1H), 4.25 (dd, 2H), 3.86-3.65 (m, 2H), 3.54 (q, 1H), 3.42 (dtd, 3H), 3.09 (d, 9H), 2.80 (d, 10H), 2.31-1.98 (m, 4H), 1.98-1.62 (m, 11H), 1.61-1.45 (m, 6H), 1.46-1.25 (m, 13H), 1.06 (d, 3H).

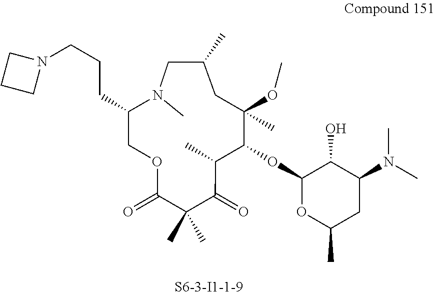

(3S,6R,8R,9R,10R)-3-(3-(azetidin-1-yl)propyl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S6-3-I1-1-9)

Prepared according to the methods of S6-3-I1-1-1 from azetidine to give the title compound as a formate salt. MS (ESI+) m/z: 598.42 [M+H]+; 1H NMR (400 MHz, Methanol-d4) δ 4.44 (d, 1H), 4.25 (d, 2H), 4.02 (t, 4H), 3.83-3.57 (m, 2H), 3.51-3.24 (m, 4H), 3.22-2.84 (m, 8H), 2.78 (s, 8H), 2.44 (p, 2H), 2.18 (s, 1H), 2.08-1.95 (m, 1H), 1.89 (s, 1H), 1.66 (s, 3H), 1.49 (tt, 6H), 1.44-1.24 (m, 13H), 1.04 (d, 3H).

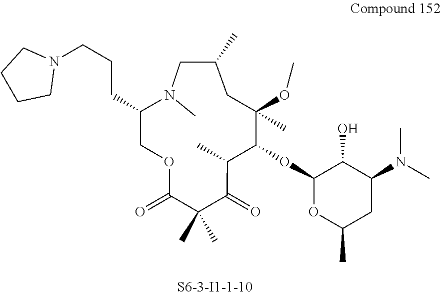

(3S,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(3-(pyrrolidin-1-yl)propyl)-1-oxa-4-azacyclotridecane-11,13-dione (S6-3-I1-1-10)

Prepared according to the methods of S6-3-I1-1-1 from pyrrolidine to give the title compound as a formate salt. MS (ESI+) m/z: 612.40 [M+H]+; 1H NMR (400 MHz, Methanol-d4) δ 4.43 (d, 1H), 4.23 (dd, 2H), 3.83-3.57 (m, 2H), 3.51-3.20 (m, 8H), 3.13 (qd, 3H), 2.98 (d, 5H), 2.77 (s, 8H), 2.15 (s, 1H), 2.10-1.95 (m, 6H), 1.83 (d, 4H), 1.62-1.43 (m, 6H), 1.44-1.22 (m, 12H), 1.03 (d, 3H).

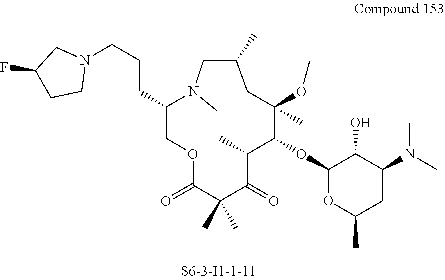

(3S,6R,8R,9R,10R)-9-(((2S,3R,4S,6R)-4-(dimethylamino)-3-hydroxy-6-methyltetrahydro-2H-pyran-2-yl)oxy)-3-(3-((R)-3-fluoropyrrolidin-1-yl)propyl)-8-methoxy-4,6,8,10,12,12-hexamethyl-1-oxa-4-azacyclotridecane-11,13-dione (S6-3-I1-1-11)

Prepared according to the methods of S6-3-I1-1-1 from (R)-3-fluoropyrrolidine to give the title compound as a formate salt. MS (ESI+) m/z: 630.28 [M+H]+; 1H NMR (400 MHz, Methanol-d4) δ 5.25 (dt, 1H), 4.45 (d, 1H), 4.24 (dd, 2H), 3.88-3.61 (m, 2H), 3.41 (dtd, 3H), 3.31 (p, 5H), 3.26-2.93 (m, 9H), 2.80 (s, 9H), 2.44-2.07 (m, 2H), 2.03 (ddd, 1H), 1.94 (s, 1H), 1.86-1.62 (m, 3H), 1.50 (d, 6H), 1.40 (d, 6H), 1.34 (dd, 6H), 1.06 (d, 3H).

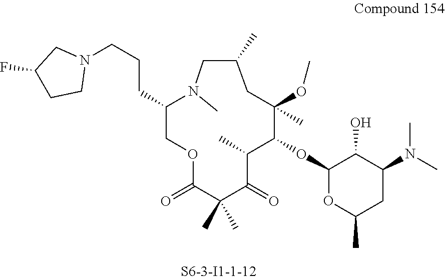

(3S,6R,8R,9R,10R)-9-(((2S,3R,4S,6R)-4-(dimethylamino)-3-hydroxy-6-methyltetrahydro-2H-pyran-2-yl)oxy)-3-(3-((S)-3-fluoropyrrolidin-1-yl)propyl)-8-methoxy-4,6,8,10,12,12-hexamethyl-1-oxa-4-azacyclotridecane-11,13-dione (S6-3-I1-1-12)

Prepared according to the methods of S6-3-I1-1-1 from (S)-3-fluoropyrrolidine to give the title compound as a formate salt. MS (ESI+) m/z: 630.27 [M+H]+; 1H NMR (400 MHz, Methanol-d4) δ 5.24 (dt, 1H), 4.45 (d, 1H), 4.24 (t, 2H), 3.88-3.61 (m, 2H), 3.41 (ddd, 3H), 3.31 (p, 5H), 3.23-2.92 (m, 8H), 2.79 m, 10H), 2.37-2.11 (m, 2H), 2.08-1.99 (m, 1H), 1.94 (s, 1H), 1.86-1.59 (m, 3H), 1.50 (d, 6H), 1.40 (d, 6H), 1.34 (dd, 6H), 1.06 (d, 3H).

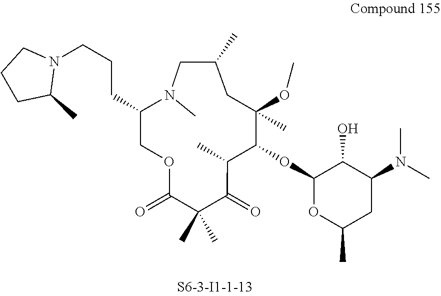

(3S,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(3-((S)-2-methylpyrrolidin-1-yl)propyl)-1-oxa-4-azacyclotridecane-11,13-dione (S6-3-I1-1-13)

Prepared according to the methods of S6-3-I1-1-1 from (S)-2-methylpyrrolidine to give the title compound as a formate salt. MS (ESI+) m/z: 626.29 [M+H]+; 1H NMR (400 MHz, Methanol-d4) δ 4.43 (d, 1H), 4.37-3.99 (m, 2H), 3.70 (ddt, 1H), 3.55 (s, 1H), 3.42 (dd, 2H), 3.35-3.14 (m, 8H), 2.99 (s, 7H), 2.73 (s, 7H), 2.23 (dt, 2H), 2.10-1.92 (m, 4H), 1.91-1.61 (m, 5H), 1.60-1.43 (m, 5H), 1.43-1.20 (m, 15H), 1.02 (s, 3H).

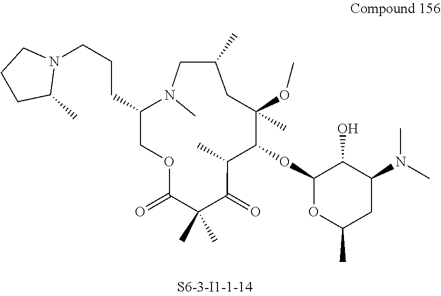

(3S,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(3-((R)-2-methylpyrrolidin-1-yl)propyl)-1-oxa-4-azacyclotridecane-11,13-dione (S6-3-I1-1-14)

Prepared according to the methods of S6-3-I1-1-1 from (R)-2-methylpyrrolidine to give the title compound as a formate salt. MS (ESI+) m/z: 626.29 [M+H]+; 1H NMR (400 MHz, Methanol-d4) δ 4.44 (d, 1H), 4.38-4.02 (m, 2H), 3.70 (ddt, 1H), 3.56 (q, 1H), 3.42 (dd, 2H), 3.36-3.18 (m, 7H), 3.00 (s, 6H), 2.74 (s, 9H), 2.25 (dq, 2H), 2.09-1.94 (m, 4H), 1.87 (s, 2H), 1.70 (dq, 3H), 1.48 (d, 5H), 1.44-1.18 (m, 15H), 1.02 (s, 3H).

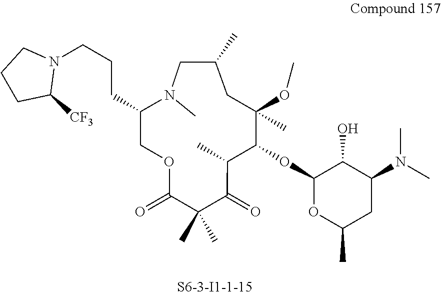

(3S,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(3-((R)-2-(trifluoromethyl)pyrrolidin-1-yl)propyl)-1-oxa-4-azacyclotridecane-11,13-dione (S6-3-I1-1-15)

Prepared according to the methods of S6-3-I1-1-1 from (R)-2-trifluoromethylpyrrolidine to give the title compound as a formate salt. MS (ESI+) m/z: 680.24 [M+H]+; 1H NMR (400 MHz, Methanol-d4) δ 4.40 (d, 1H), 4.25 (d, 2H), 3.86-3.55 (m, 2H), 3.46-3.28 (m, 2H), 3.26-3.14 (m, 2H), 2.97 (dd, 8H), 2.79 (s, 1H), 2.70-2.47 (m, 8H), 2.42 (td, 2H), 2.21 (s, 1H), 2.12-1.97 (m, 2H), 1.96-1.76 (m, 5H), 1.71 (t, 1H), 1.65-1.17 (m, 20H), 1.06 (s, 3H).

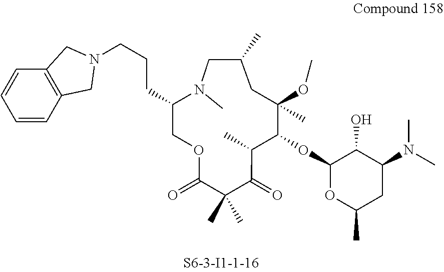

(3S,6R,8R,9R,10R)-9-(((2S,3R,4S,6R)-4-(dimethylamino)-3-hydroxy-6-methyltetrahydro-2H-pyran-2-yl)oxy)-3-(3-(isoindolin-2-yl)propyl)-8-methoxy-4,6,8,10,12,12-hexamethyl-1-oxa-4-azacyclotridecane-11,13-dione (S6-3-I1-1-16)

Prepared according to the methods of S6-3-I1-1-1 from isoindoline to give the title compound as a formate salt. (ESI+) m/z: 660.29 [M+H]+; 1H NMR (400 MHz, Methanol-d4) δ 7.27 (p, 4H), 4.46 (d, 1H), 4.26 (dd, 2H), 4.11 (s, 4H), 3.89-3.64 (m, 2H), 3.53-3.27 (m, 5H), 3.18-2.91 (m, 9H), 2.80 (s, 7H), 2.20 (s, 1H), 2.10-1.92 (m, 2H), 1.75 (ddd, 3H), 1.47 (d, 6H), 1.44-1.29 (m, 12H), 1.06 (d, 3H).

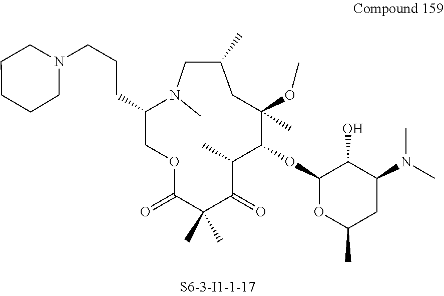

(3S,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(3-(piperidin-1-yl)propyl)-1-oxa-4-azacyclotridecane-11,13-dione (S6-3-I1-1-17)

Prepared according to the methods of S6-3-I1-1-1 from piperidine to give the title compound as a formate salt. MS (ESI+) m/z: 626.56 [M+H]+; 1H NMR (400 MHz, Methanol-d4) δ 4.45 (d, 1H), 4.36-4.04 (m, 2H), 3.82-3.62 (m, 2H), 3.43 (dd, 2H), 3.31 (dt, 3H), 2.97 (d, 11H), 2.76 (s, 8H), 2.09-1.95 (m, 2H), 1.95-1.69 (m, 8H), 1.63 (s, 3H), 1.48 (t, 5H), 1.45-1.19 (m, 13H), 1.04 (s, 3H).

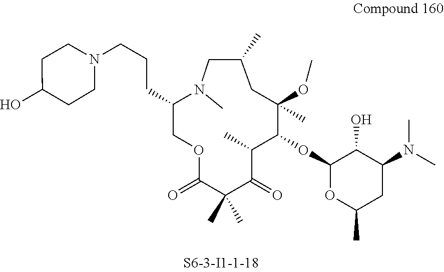

(3S,6R,8R,9R,10R)-9-(((2S,3R,4S,6R)-4-(dimethylamino)-3-hydroxy-6-methyltetrahydro-2H-pyran-2-yl)oxy)-3-(3-(4-hydroxypiperidin-1-yl)propyl)-8-methoxy-4,6,8,10,12,12-hexamethyl-1-oxa-4-azacyclotridecane-11,13-dione (S6-3-I1-1-18)

Prepared according to the methods of S6-3-I1-1-1 from 4-hydroxypiperidine to give the title compound as a formate salt. MS (ESI+) m/z: 642.36 [M+H]+, 1H NMR (400 MHz, Methanol-d4) δ 4.44 (d, 1H), 4.23 (t, 2H), 3.93 (s, 1H), 3.85-3.65 (m, 2H), 3.54-3.22 (m, 9H), 3.03 (d, 10H), 2.81 (s, 7H), 2.20 (s, 1H), 2.13-1.98 (m, 3H), 1.98-1.63 (m, 6H), 1.45 (s, 6H), 1.44-1.23 (m, 12H), 1.05 (d, 3H).

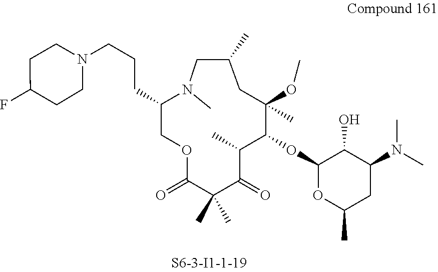

(3S,6R,8R,9R,10R)-9-(((2S,3R,4S,6R)-4-(dimethylamino)-3-hydroxy-6-methyltetrahydro-2H-pyran-2-yl)oxy)-3-(3-(4-fluoropiperidin-1-yl)propyl)-8-methoxy-4,6,8,10,12,12-hexamethyl-1-oxa-4-azacyclotridecane-11,13-dione (S6-3-I1-1-19)

Prepared according to the methods of S6-3-I1-1-1 from 4-fluoropiperidine to give the title compound as a formate salt. MS (ESI+) m/z: 644.36 [M+H]+; 1H NMR (400 MHz, Methanol-d4) δ 4.79 (d, 1H), 4.45 (d, 1H), 4.24 (t, 2H), 3.87-3.63 (m, 2H), 3.42 (dtd, 3H), 3.31 (p, 5H), 3.12-2.90 (m, 10H), 2.81 (s, 9H), 2.26-1.94 (m, 6H), 1.77 (d, 3H), 1.48 (s, 5H), 1.44-1.26 (m, 12H), 1.06 (d, 3H).

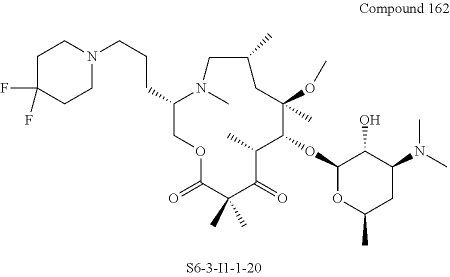

(3S,6R,8R,9R,10R)-3-(3-(4,4-difluoropiperidin-1-yl)propyl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S6-3-I1-1-20)

Prepared according to the methods of S6-3-I1-1-1 from 4-difluoropiperidine to give the title compound as a formate salt. MS (ESI+) m/z: 662.38 [M+H]+; 1H NMR (400 MHz, Methanol-d4) δ 4.45 (d, 1H), 4.24 (t, 2H), 3.88-3.64 (m, 2H), 3.56-3.34 (m, 3H), 3.30 (p, 4H), 3.04 (d, 6H), 2.81 (s, 7H), 2.73-2.61 (m, 4H), 2.55 (t, 2H), 2.20 (s, 1H), 2.02 (dp, 6H), 1.75 (dd, 2H), 1.50 (d, 6H), 1.45-1.24 (m, 12H), 1.06 (d, 3H).

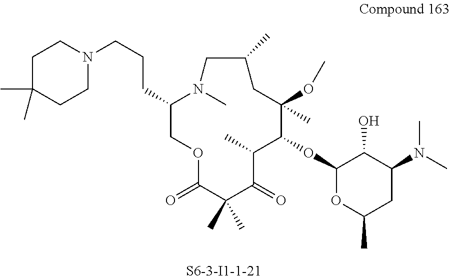

(3S,6R,8R,9R,10R)-9-(((2S,3R,4S,6R)-4-(dimethylamino)-3-hydroxy-6-methyltetrahydro-2H-pyran-2-yl)oxy)-3-(3-(4,4-dimethylpiperidin-1-yl)propyl)-8-methoxy-4,6,8,10,12,12-hexamethyl-1-oxa-4-azacyclotridecane-11,13-dione (S6-3-I1-1-21)

Prepared according to the methods of S6-3-I1-1-1 from 4-dimethylpiperidine to give the title compound as a formate salt. MS (ESI+) m/z: 654.44 [M+H]+; 1H NMR (400 MHz, Methanol-d4) δ 4.45 (d, 1H), 4.38-4.05 (m, 2H), 3.83-3.58 (m, 2H), 3.44 (dd, 2H), 3.36-3.27 (m, 12H), 3.04 (s, 9H), 2.78 (s, 6H), 2.18 (s, 1H), 2.01 (ddd, 1H), 1.97-1.69 (m, 3H), 1.64 (t, 4H), 1.51 (d, 5H), 1.45-1.25 (m, 11H), 1.05 (s, 8H).

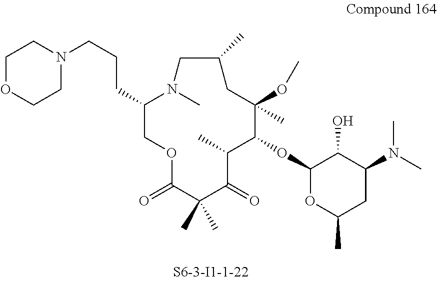

(3S,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(3-morpholinopropyl)-1-oxa-4-azacyclotridecane-11,13-dione (S6-3-I1-1-22)

Prepared according to the methods of S6-3-I1-1-1 from morpholine to give the title compound as a formate salt. MS (ESI+) m/z: 628.40 [M+H]+; 1H NMR (400 MHz, Methanol-d4) δ 4.46 (d, 1H), 4.25 (t, 2H), 3.72 (t, 6H), 3.42 (ddd, 5H), 3.05 (d, 7H), 2.80 (s, 7H), 2.51 (dt, 6H), 2.22 (s, 1H), 2.10-1.98 (m, 1H), 1.94 (s, 1H), 1.73 (d, 2H), 1.65-1.45 (m, 7H), 1.45-1.26 (m, 12H), 1.07 (d, 3H).

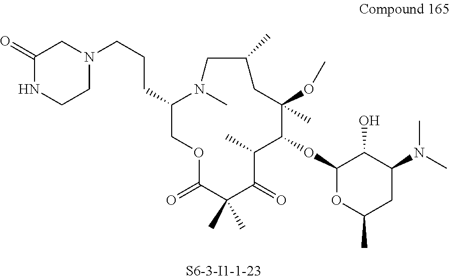

(3S,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(3-(3-oxopiperazin-1-yl)propyl)-1-oxa-4-azacyclotridecane-11,13-dione (S6-3-I1-1-23)

Prepared according to the methods of S6-3-I1-1-1 from piperazin-2-one to give the title compound as a formate salt. MS (ESI+) m/z: 641.40 [M+H]+; 1H NMR (400 MHz, Methanol-d4) δ 4.45 (d, 1H), 4.25 (t, 2H), 3.88-3.63 (m, 2H), 3.53-3.31 (m, 8H), 3.18-2.93 (m, 9H), 2.81 (s, 7H), 2.76-2.62 (m, 2H), 2.53 (q, 2H), 2.20 (s, 1H), 2.10-1.88 (m, 2H), 1.88-1.66 (m, 2H), 1.66-1.44 (m, 7H), 1.44-1.25 (m, 12H), 1.06 (d, 3H).

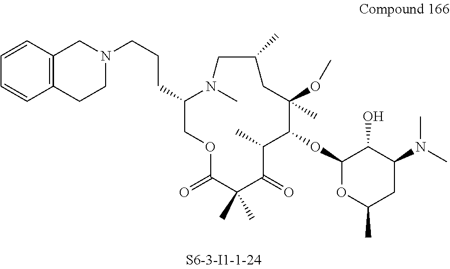

(3S,6R,8R,9R,10R)-3-(3-(3,4-dihydroisoquinolin-2(1H)-yl)propyl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S6-3-I1-1-24)

Prepared according to the methods of S6-3-I1-1-1 from 1,2,3,4-tetrahydroisoquinoline to give the title compound as a formate salt. MS (ESI+) m/z: 674.33 [M+H]+; 1H NMR (400 MHz, Methanol-d4) δ 7.28-6.98 (m, 4H), 4.45 (d, 1H), 4.27 (dd, 2H), 3.98-3.62 (m, 4H), 3.55-3.34 (m, 4H), 3.19-2.85 (m, 11H), 2.77 (s, 10H), 2.18 (d, 1H), 2.01 (ddd, 2H), 1.91-1.65 (m, 3H), 1.65-1.44 (m, 6H), 1.44-1.25 (m, 12H), 1.05 (d, 3H).

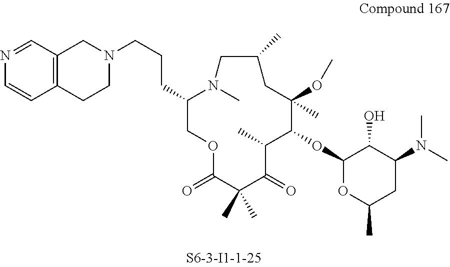

(3S,6R,8R,9R,10R)-3-(3-(3,4-dihydro-2,7-naphthyridin-2(1H)-yl)propyl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione (S6-3-I1-1-25)

Prepared according to the methods of S6-3-I1-1-1 from 1,2,3,4-tetrahydro-2,7-naphthyridine to give the title compound as a formate salt. MS (ESI+) m/z: 675.28 [M+H]+; 1H NMR (400 MHz, Methanol-d4) δ 8.32 (s, 1H), 8.26 (d, 1H), 7.16 (s, 1H), 4.45 (d, 1H), 4.27 (dd, 2H), 3.89-3.64 (m, 4H), 3.52-3.32 (m, 4H), 2.99 (dd, 9H), 2.89-2.75 (m, 9H), 2.66 (t, 2H), 2.20 (s, 1H), 2.03 (ddd, 2H), 1.89-1.64 (m, 3H), 1.63-1.45 (m, 6H), 1.45-1.26 (m, 12H), 1.05 (d, 3H).

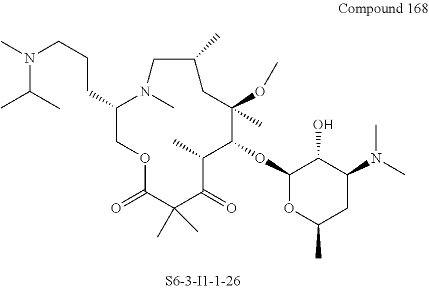

(3S,6R,8R,9R,10R)-9-(((2S,3R,4S,6R)-4-(dimethylamino)-3-hydroxy-6-methyltetrahydro-2H-pyran-2-yl)oxy)-3-(3-(isopropyl(methyl)amino)propyl)-8-methoxy-4,6,8,10,12,12-hexamethyl-1-oxa-4-azacyclotridecane-11,13-dione (S6-3-I1-1-26)

Prepared according to the methods of S6-3-I1-1-1 from N-methylisopropylamine to give the title compound as a formate salt. MS (ESI+) m/z: 614.48 [M+H]+; ¹H NMR (400 MHz, MeOD-d4) δ 4.45 (d, 1H), 4.28 (d, 1H), 4.20 (d, 1H), 3.88-3.65 (m, 2H), 3.56 (hept, 1H), 3.51-3.23 (m, 4H), 3.05 (t, 7H), 2.79 (s, 8H), 2.72 (s, 3H), 2.17 (s, 1H), 2.02 (ddd, 1H), 1.84 (d, 4H), 1.67-1.41 (m, 7H), 1.45-1.19 (m, 19H), 1.05 (d, 3H).

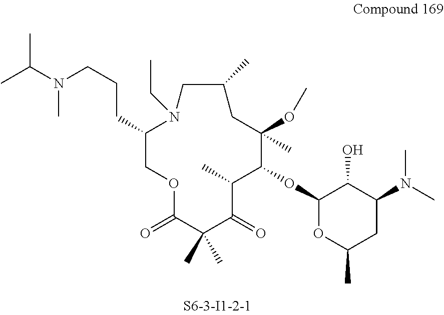

(3S,6R,8R,9R,10R)-9-(((2S,3R,4S,6R)-4-(dimethylamino)-3-hydroxy-6-methyltetrahydro-2H-pyran-2-yl)oxy)-4-ethyl-3-(3-(isopropyl(methyl)amino)propyl)-8-methoxy-6,8,10,12,12-pentamethyl-1-oxa-4-azacyclotridecane-11,13-dione (S6-3-I1-2-1)

Prepared according to the methods of S6-3-I1-1-1 from S2-1-I1-2 and N-methylisopropylamine to give the title compound as a formate salt. MS (ESI+) m/z: 628.50 [M+H]+; 1HNMR (400 MHz, Methanol-d4) δ 4.51-4.65 (s, 1H), 4.42 (d, 1H), 4.25 (s, 1H), 4.01 (t, 2H), 3.69 (ddt, 1H), 3.53 (s, 1H), 3.41 (dd, 2H), 3.22 (d, 2H), 3.02 (s, 4H), 2.82 (d, 2H), 2.72 (s, 10H), 2.34 (s, 1H), 2.27-2.03 (m, 2H), 1.97 (ddd, 1H), 1.69 (d, 4H), 1.56-1.17 (m, 24H), 1.16-0.74 (m, 6H).

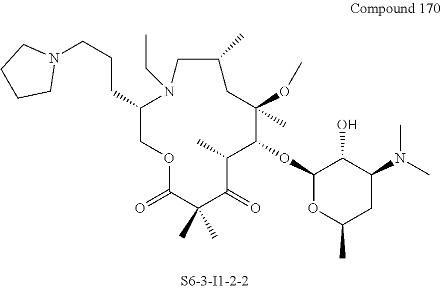

(3S,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-3-(3-(pyrrolidin-1-yl)propyl)-1-oxa-4-azacyclotridecane-11,13-dione (S6-3-I1-2-2)

Prepared according to the methods of S6-3-I1-1-1 from S2-1-I1-2 and pyrrolidine to give the title compound as a formate salt. MS (ESI+) m/z: 626.41 [M+H]+; 1H NMR (400 MHz, Methanol-d4) δ 4.51-4.65 (s, 1H), 4.44 (d, 1H), 4.26 (s, 1H), 3.95 (s, 1H), 3.81-3.52 (m, 2H), 3.50-3.19 (m, 13H), 3.21-2.88 (m, 6H), 2.78 (s, 7H), 2.20-1.94 (m, 6H), 1.84 (s, 4H), 1.58-1.23 (m, 18H), 1.04 (s, 3H).

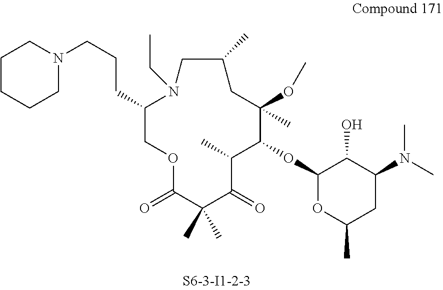

(3S,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-3-(3-(piperidin-1-yl)propyl)-1-oxa-4-azacyclotridecane-11,13-dione (S6-3-I1-2-3)

Prepared according to the methods of S6-3-I1-1-1 from S2-1-I1-2 and piperidine to give the title compound as a formate salt. MS (ESI+) m/z: 640.46 [M+H]+; 1H NMR (400 MHz, Methanol-d4) δ 4.51-4.65 (s, 1H), 4.44 (d, 1H), 4.26 (s, 1H), 3.95 (s, 1H), 3.83-3.52 (m, 2H), 3.52-3.27 (m, 12H), 3.28-2.88 (m, 7H), 2.79 (s, 7H), 2.19 (s, 1H), 2.02 (ddd, 1H), 1.83 (p, 7H), 1.59-1.17 (m, 20H), 1.05 (s, 4H).

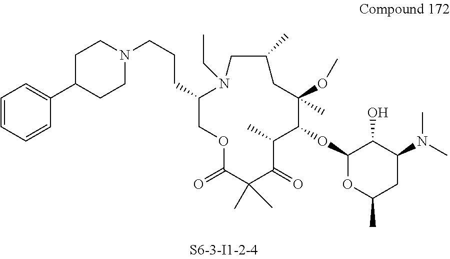 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCC(c2ccccc2)CC1]

(3S,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-3-(3-(4-phenylpiperidin-1-yl)propyl)-1-oxa-4-azacyclotridecane-11,13-dione (S6-3-I1-2-4)

Prepared according to the methods of S6-3-I1-1-1 from S2-1-I1-2 and 4-phenylpiperidine to give the title compound as a formate salt. MS (ESI+) m/z: 716.34 [M+H]+; ¹H NMR (400 MHz, MeOD-d4): δ 7.24 (td, 5H), 4.62 (s, 1H), 4.41 (s, 2H), 4.27 (s, 1H), 4.01 (s, 1H), 3.74-3.50 (m, 2H), 3.44 (d, 3H), 3.15 (d, 3H), 3.05 (s, 2H), 2.84 (s, 3H), 2.63 (d, 10H), 2.24 (d, 3H), 1.56-2.00 (m, 9H), 1.54-1.22 (m, 20H), 1.09 (s, 3H), 0.90 (s, 1H).

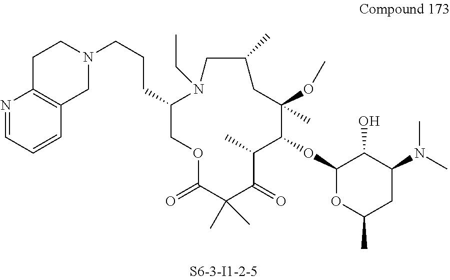 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCc2ncccc2C1]

(3S,6R,8R,9R,10R)-3-(3-(7,8-dihydro-1,6-naphthyridin-6(5H)-yl)propyl)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-1-oxa-4-azacyclotridecane-11,13-dione (S6-3-I1-2-5)

Prepared according to the methods of S6-3-I1-1-1 from S2-1-I1-2 and 5,6,7,8-tetrahydro-1,6-naphthyridine to give the title compound as a formate salt. MS (ESI+) m/z: 689.36 [M+H]+; 1H NMR (400 MHz, MeOD-d4): δ 8.42 (s, 3H), 8.33 (dd, 1H), 7.67-7.46 (m, 1H), 7.30-7.06 (m, 1H), 4.59 (t, 1H), 4.51-4.37 (m, 2H), 4.25 (d, 1H), 4.00 (s, 1H), 3.77 (s, 2H), 3.75-3.65 (m, 1H), 3.55 (s, 1H), 3.47-3.32 (m, 3H), 3.12 (d, 2H), 2.99 (td, 7H), 2.89-2.82 (m, 1H), 2.81 (d, 6H), 2.76-2.66 (m, 2H), 2.14 (s, 1H), 2.07-1.98 (m, 1H), 1.94 (s, 1H), 1.87-1.69 (m, 3H), 1.56-1.47 (m, 1H), 1.44 (s, 3H), 1.40-1.23 (m, 16H), 1.04 (d, 3H).

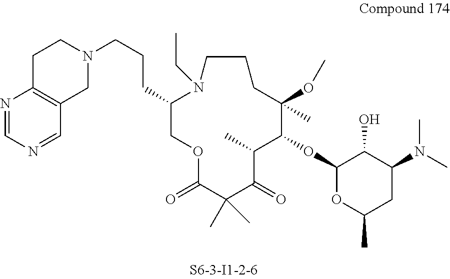 [SMILES: CCN1CCC[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCc2ncncc2C1]

(3S,8R,9R,10R)-3-(3-(7,8-dihydropyrido[4,3-d]pyrimidin-6(5H)-yl)propyl)-9-(((2S,3R,4S,6R)-4-(dimethylamino)-3-hydroxy-6-methyltetrahydro-2H-pyran-2-yl)oxy)-4-ethyl-8-methoxy-8,10,12,12-tetramethyl-1-oxa-4-azacyclotridecane-11,13-dione (S6-3-I1-2-6)

Prepared according to the methods of S6-3-I1-1-1 from S2-1-I1-2 and 5,6,7,8-tetrahydropyrido[4,3-d]pyrimidine to give the title compound as a formate salt. MS (ESI+) m/z: 690.33 [M+H]+; 1H NMR (400 MHz, MeOD-d4): δ 8.90 (s, 1H), 8.51 (s, 1H), 8.46 (s, 2H), 4.60 (t, 1H), 4.45 (dd, 2H), 4.26 (d, 1H), 3.99 (s, 1H), 3.71 (s, 3H), 3.55 (s, 1H), 3.50-3.33 (m, 3H), 3.25-3.06 (m, 2H), 3.00 (d, 5H), 2.95-2.83 (m, 3H), 2.81 (s, 6H), 2.68 (tt, 2H), 2.18 (d, 1H), 2.08-1.99 (m, 1H), 1.95 (s, 1H), 1.88-1.68 (m, 3H), 1.58-1.49 (m, 1H), 1.45 (s, 3H), 1.42-1.20 (m, 16H), 1.04 (d, 3H).

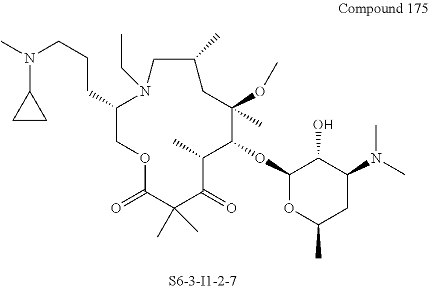

(3S,6R,8R,9R,10R)-3-(3-(cyclopropyl(methyl)amino)propyl)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-1-oxa-4-azacyclotridecane-11,13-dione (S6-3-I1-2-7)

Prepared according to the methods of S6-3-I1-1-1 from S2-1-I1-2 and N-methylcyclopropaneamine to give the title compound as a formate salt. MS (ESI+) m/z: 626.01 [M+H]+; ¹H NMR (400 MHz, MeOD-d4): δ 4.59 (s, 1H), 4.50-4.35 (m, 2H), 4.26 (d, 1H), 3.91 (s, 1H), 3.79-3.68 (m, 1H), 3.57 (s, 1H), 3.42 (dtd, 3H), 3.20 (s, 1H), 3.03 (s, 3H), 2.82 (s, 9H), 2.57 (s, 3H), 2.22-1.99 (m, 3H), 1.92-1.69 (m, 4H), 1.58-1.30 (m, 23H), 1.07 (d, 3H), 0.69 (d, 4H).

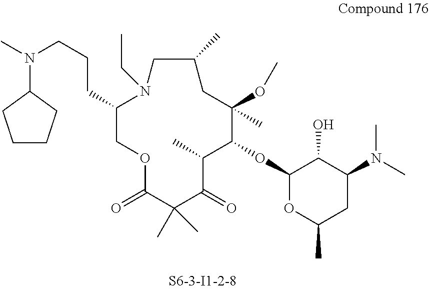

(3S,6R,8R,9R,10R)-3-(3-(cyclopentyl(methyl)amino)propyl)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-1-oxa-4-azacyclotridecane-11,13-dione (S6-3-I1-2-8)

Prepared according to the methods of S6-3-I1-1-1 from S2-1-I1-2 and N-methylcyclopentanamine to give the title compound as a formate salt. MS (ESI+) m/z: 654.08 [M+H]+; ¹H NMR (400 MHz, Methanol-d₄): δ4.59 (s, 1H), 4.44 (d, 2H), 4.25 (s, 1H), 3.94 (s, 1H), 3.73 (dtd, 1H), 3.54 (p, 2H), 3.45 (dd, 1H), 3.37 (ddd, 2H), 3.25-2.86 (m, 7H), 2.79 (d, 10H), 2.30-1.95 (m, 4H), 1.96-1.58 (m, 11H), 1.60-1.14 (m, 21H), 1.05 (s, 3H).

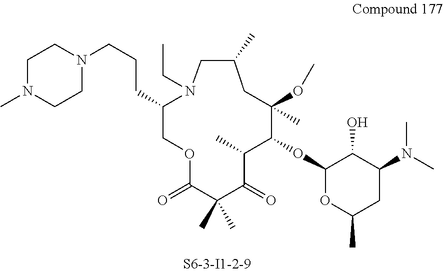

(3S,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-3-(3-(4-methylpiperazin-1-yl)propyl)-1-oxa-4-azacyclotridecane-11,13-dione (S6-3-I1-2-9)

Prepared according to the methods of S6-3-I1-1-1 from S2-1-I1-2 and N-methylpiperazine to give the title compound as a formate salt. MS (ESI+) m/z: 655.34 [M+H]+; 1H NMR (400 MHz, Methanol-d4) δ 4.60 (s, 1H), 4.42 (t, 2H), 4.26 (s, 1H), 3.93 (s, 1H), 3.80-3.66 (m, 1H), 3.58 (s, 1H), 3.49-3.27 (m, 4H), 3.27-2.36 (m, 25H), 2.17 (s, 1H), 2.07-1.96 (m, 1H), 1.97-1.19 (m, 25H), 1.06 (d, 3H).

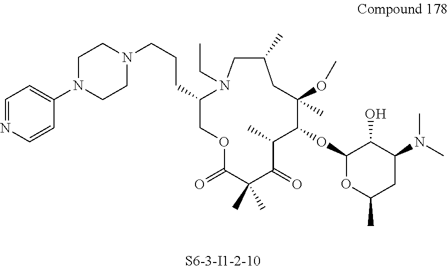

(3S,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-3-(3-(4-(pyridin-4-yl)piperazin-1-yl)propyl)-1-oxa-4-azacyclotridecane-11,13-dione (S6-3-I1-2-10)

Prepared according to the methods of S6-3-I1-1-1 from S2-1-I1-2 and 1-(pyridin-4-yl)piperazine to give the title compound as a formate salt. MS (ESI+) m/z: 718.31 [M+H]+; 1H NMR (400 MHz, Methanol-d4) δ 8.13 (d, 7.11 (s, 2H), 4.61 (s, 1H), 4.43 (q, 2H), 4.25 (s, 1H), 3.91 (d, 1H), 3.79-3.49 (m, 6H), 3.50-3.31 (m, 3H), 3.14 (d, 2H), 3.02 (s, 3H), 2.79 (d, 7H), 2.62 (q, 4H), 2.50 (hept, 2H), 2.15 (d, 1H), 2.07-1.85 (m, 2H), 1.84-1.18 (m, 25H), 1.06 (d, 3H).

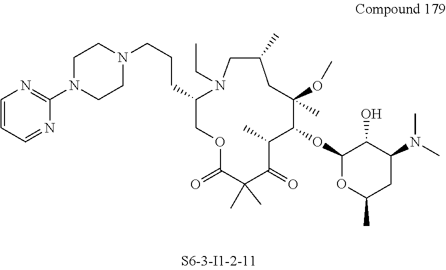 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCN(c2ncccn2)CC1]

(3S,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-3-(3-(4-(pyrimidin-2-yl)piperazin-1-yl)propyl)-1-oxa-4-azacyclotridecane-11,13-dione (S6-3-I1-2-11)

Prepared according to the methods of S6-3-I1-1-1 from S2-1-I1-2 and 2-(piperazin-1-yl)pyrimidine to give the title compound as a formate salt. MS (ESI+) m/z: 719.35 [M+H]+; ¹H NMR (400 MHz, MeOH-d4): δ 8.33 (d, 2H), 6.62 (t, 1H), 4.62 (s, 1H), 4.44 (d, 2H), 4.28 (d, 1H), 4.01 (s, 1H), 3.85 (t, 4H), 3.73 (ddt, 1H), 3.59 (s, 1H), 3.42 (ddd, 3H), 3.16 (d, 2H), 3.03 (s, 3H), 2.80 (s, 7H), 2.60 (dt, 6H), 2.19 (s, 1H), 2.03 (ddd, 1H), 1.93 (s, 1H), 1.85-1.63 (m, 3H), 1.63-1.20 (m, 21H), 1.07 (d, 3H).

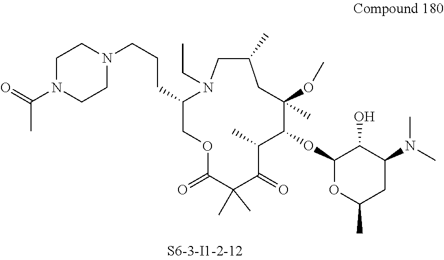 [SMILES: CCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCN(C(C)=O)CC1]

(3S,6R,8R,9R,10R)-3-(3-(4-acetylpiperazin-1-yl)propyl)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-1-oxa-4-azacyclotridecane-11,13-dione (S6-3-I1-2-12)

Prepared according to the methods of S6-3-I1-1-1 from S2-1-I1-2 and 1-acetylpiperizine to give the title compound as a formate salt. MS (ESI+) m/z: 683.3 [M+H]+; ¹H NMR (400 MHz, MeOH-d4) δ 4.60 (d, 1H), 4.43 (t, 2H), 4.27 (d, 1H), 3.92 (d, 1H), 3.82-3.67 (m, 1H), 3.66-3.51 (m, 5H), 3.41 (dtd, 3H), 0.27-3.07 (m, 2H), 3.03 (s, 3H), 2.81 (s, 7H), 2.51 (dq, 6H), 2.22 (d, 1H), 2.10 (s, 3H), 2.07-1.97 (m, 1H), 1.97-1.84 (m, 1H), 1.84-1.49 (m, 5H), 1.48-1.25 (m, 19H), 1.07 (d, 3H).

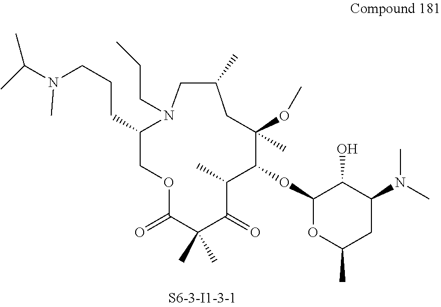

(3S,6R,8R,9R,10R)-9-(((2S,3R,4S,6R)-4-(dimethylamino)-3-hydroxy-6-methyltetrahydro-2H-pyran-2-yl)oxy)-3-(3-(isopropyl(methyl)amino)propyl)-8-methoxy-6,8,10,12,12-pentamethyl-4-propyl-1-oxa-4-azacyclotridecane-11,13-dione (S6-3-I1-3-1)

Prepared according to the methods of S6-3-I1-1-1 from S2-1-I1-3 and propionaldehdye to give 10.6 mg of the title compound as a formate salt. MS (ESI+) m/z: 214.8 [M+3H]3+, 321.7 [M+2H]2+, 642.3 [M+H]+; 1H NMR (400 MHz, Methanol-d4) δ 8.52 (s, 2H), 4.44 (d, 1H), 4.40-4.25 (m, 1H), 4.12-4.00 (m, 1H), 4.00-3.84 (m, 1H), 3.79-3.66 (m, 1H), 3.66-3.51 (m, 2H), 3.44 (t, 2H), 3.19-2.91 (m, 4H), 2.91-2.67 (m, 11H), 2.66-2.48 (m, 1H), 2.45-2.08 (m, 3H), 2.00 (dd, 1H), 1.94-1.57 (m, 5H), 1.57-1.41 (m, 7H), 1.39-1.23 (m, 17H), 1.23-0.82 (m, 7H).

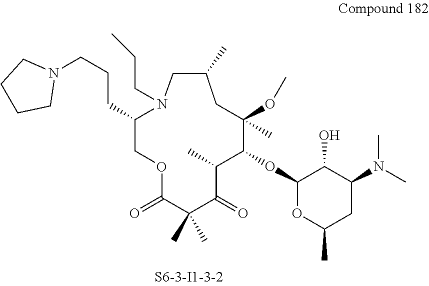

(3S,6R,8R,9R,10R)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propyl-3-(3-(pyrrolidin-1-yl)propyl)-1-oxa-4-azacyclotridecane-11,13-dione (S6-3-I1-3-2)

Prepared according to the methods of S6-3-I1-1-1 from S2-1-I1-3 and pyrrolidine to give 12.7 mg of the title compound as a formate salt. MS (ESI+) m/z: 214.2 [M+3H]3+, 320.7 [M+2H]2+, 640.3 [M+H]+; 1H NMR (400 MHz, Methanol-d4) δ 8.52 (s, 3H), 4.44 (d, 1H), 4.40-4.21 (m, 1H), 4.10-3.80 (m, 2H), 3.71 (dd, 1H), 3.66-3.52 (m, 1H), 3.44 (t, 2H), 3.21-2.90 (m, 6H), 2.80 (s, 9H), 2.65-2.45 (m, 1H), 2.45-2.12 (m, 3H), 2.12-1.90 (m, 6H), 1.91-1.59 (m, 5H), 1.43 (s, 7H), 1.42-1.22 (m, 14H), 1.22-0.72 (m, 8H).

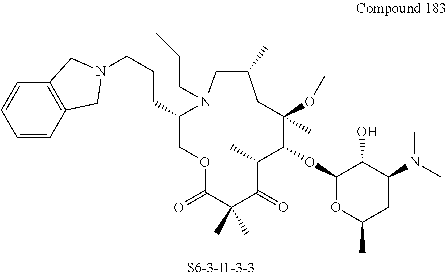

(3S,6R,8R,9R,10R)-9-(((2S,3R,4S,6R)-4-(dimethylamino)-3-hydroxy-6-methyltetrahydro-2H-pyran-2-yl)oxy)-3-(3-(isoindolin-2-yl)propyl)-8-methoxy-6,8,10,12,12-pentamethyl-4-propyl-1-oxa-4-azacyclotridecane-11,13-dione (S6-3-I1-3-3)

Prepared according to the methods of S6-3-I1-1-1 from S2-1-I1-3 and isoindoline to give 11.9 mg of the title compound as a formate salt. MS (ESI+) m/z: 230.1 [M+3H]3+, 344.7 [M+2H]2+, 688.3 [M+H]+; 1H NMR (400 MHz, Methanol-d4) δ 8.54 (s, 2H), 7.26 (s, 4H), 4.69-4.20 (m, 3H), 4.20-3.86 (m, 6H), 3.69 (dd, 1H), 3.65-3.51 (m, 1H), 3.48-3.34 (m, 2H), 3.28-3.09 (m, 2H), 3.00 (s, 2H), 2.96-2.78 (m, 4H), 2.78-2.51 (m, 7H), 2.51-2.10 (m, 2H), 2.00-1.89 (m, 2H), 1.87-1.58 (m, 5H), 1.58-1.19 (m, 19H), 1.14-0.82 (m, 7H).

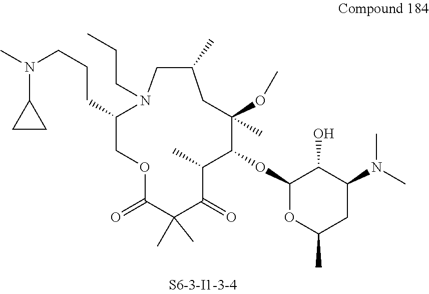

(3S,8R,9R,10R)-3-(3-(cyclopropyl(methyl)amino)propyl)-9-(((2S,3R,4S,6R)-4-(dimethylamino)-3-hydroxy-6-methyltetrahydro-2H-pyran-2-yl)oxy)-8-methoxy-8,10,12,12-tetramethyl-4-propyl-1-oxa-4-azacyclotridecane-11,13-dione (S6-3-I1-3-4)

Prepared according to the methods of S6-3-I1-1-1 from S2-1-I1-3 and N-methylcyclopropaneamine to give the title compound as a formate salt. MS (ESI+) m/z: 640.03 [M+H]+; ¹H NMR (400 MHz, Methanol-d₄): δ 4.57 (s, 1H), 4.42 (dd, 2H), 4.25 (s, 1H), 3.90 (s, 1H), 3.78-3.64 (m, 1H), 3.53-3.32 (m, 3H), 3.22 (d, 2H), 3.08-2.91 (m, 3H), 2.81 (s, 9H), 2.53 (s, 4H), 2.18 (s, 1H), 2.11-1.88 (m, 3H), 1.73 (s, 4H), 1.61-1.20 (m, 19H), 1.06 (t, 6H), 0.78-0.46 (m, 4H).

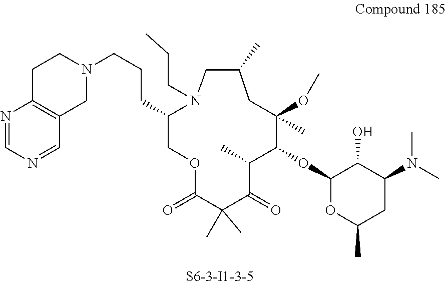 [SMILES: CCCN1C[C@H](C)C[C@@](C)(OC)[C@H](O[C@@H]2O[C@H](C)C[C@H](N(C)C)[C@H]2O)[C@@H](C)C(=O)C(C)(C)C(=O)OC[C@@H]1CCCN1CCc2ncncc2C1]

(3S,6R,8R,9R,10R)-3-(3-(7,8-dihydropyrido[4,3-d]pyrimidin-6(5H)-yl)propyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propyl-1-oxa-4-azacyclotridecane-11,13-dione (S6-3-I1-3-5)

Prepared according to the methods of S6-3-I1-1-1 from S2-1-I1-3 and 5,6,7,8-tetrahydropyrido[4,3-d]pyrimidine to give the title compound as a formate salt. MS (ESI+) m/z: 703.98 [M+H]+; ¹H NMR (400 MHz, Methanol-d₄): δ 8.91 (s, 1H), 8.52 (s, 1H), 4.57 (d, 1H), 4.46 (dd, 2H), 4.25 (s, 1H), 3.98 (s, 1H), 3.81-3.64 (m, 3H), 3.51-3.33 (m, 3H), 3.17 (s, 2H), 3.01 (d, 5H), 2.94-2.85 (m, 3H), 2.82 (s, 7H), 2.69 (hept, 2H), 2.18 (s, 1H), 2.09-1.89 (m, 3H), 1.76 (ddt, 4H), 1.57-1.25 (m, 19H), 1.09-0.92 (m, 6H).

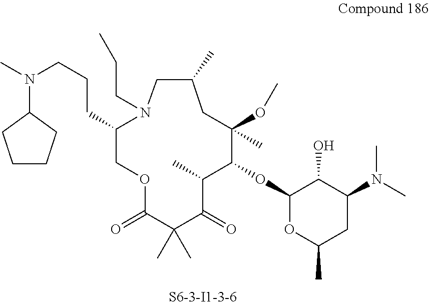

(3S,8R,9R,10R)-3-(3-(cyclopentyl(methyl)amino)propyl)-9-(((2S,3R,4S,6R)-4-(dimethylamino)-3-hydroxy-6-methyltetrahydro-2H-pyran-2-yl)oxy)-8-methoxy-8,10,12,12-tetramethyl-4-propyl-1-oxa-4-azacyclotridecane-11,13-dione (S6-3-I1-3-6)

Prepared according to the methods of S6-3-I1-1-1 from S2-1-I1-3 and N-methylcyclopentanamine to give the title compound as a formate salt. MS (ESI+) m/z: 668.06 [M+H]+; ¹H NMR (400 MHz, Methanol-d₄): δ 4.58 (s, 1H), 4.44 (d, 2H), 3.95 (s, 1H), 3.82-3.64 (m, 1H), 3.65-3.33 (m, 4H), 3.19-2.91 (m, 5H), 2.80 (d, 11H), 2.40-1.93 (m, 6H), 1.93-1.61 (m, 11H), 1.61-1.17 (m, 20H), 1.02 (s, 7H).

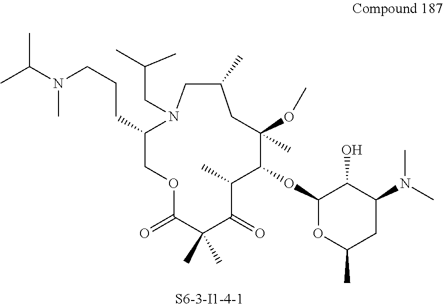

(3S,6R,8R,9R,10R)-9-(((2S,3R,4S,6R)-4-(dimethylamino)-3-hydroxy-6-methyltetrahydro-2H-pyran-2-yl)oxy)-4-isobutyl-3-(3-(isopropyl(methyl)amino)propyl)-8-methoxy-6,8,10,12,12-pentamethyl-1-oxa-4-azacyclotridecane-11,13-dione (S6-3-I1-4-1)

Prepared according to the methods of S6-3-I1-1-1 from S2-1-I1-4 and N-methylisopropylamine to give the title compound as a formate salt. MS (ESI+) m/z: 656.34 [M+H]+; 1H NMR (400 MHz, Methanol-d4) δ 4.45 (d, 2H), 4.05 (d, 1H), 3.85 (t, 1H), 3.77-3.51 (m, 3H), 3.52-3.42 (m, 1H), 3.17-3.01 (m, 3H), 2.89-2.67 (m, 12H), 2.46-2.19 (m, 3H), 2.19-1.94 (m, 3H), 1.92-1.46 (m, 10H), 1.38-1.23 (m, 18H), 1.07 (dd, 6H), 0.90 (t, 6H).

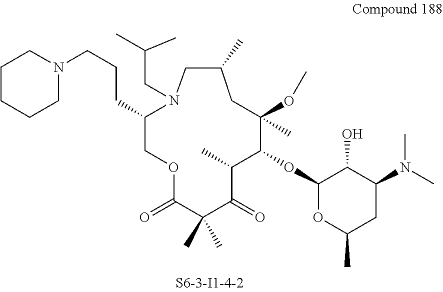

(3S,6R,8R,9R,10R)-9-(((2S,3R,4S,6R)-4-(dimethylamino)-3-hydroxy-6-methyltetrahydro-2H-pyran-2-yl)oxy)-4-isobutyl-8-methoxy-6,8,10,12,12-pentamethyl-3-(3-(piperidin-1-yl)propyl)-1-oxa-4-azacyclotridecane-11,13-dione (S6-3-I1-4-2)

Prepared according to the methods of S6-3-I1-1-1 from S2-1-I1-4 and piperidine to give the title compound as a formate salt. MS (ESI+) m/z: 668.29 [M+H]+; 1H NMR (400 MHz, Acetonitrile-d3) δ 4.82 (m, 1H), 4.55-4.34 (m, 2H), 4.05 (d, 1H), 3.85 (t, 1H), 3.79-3.56 (m, 2H), 3.45 (dd, 1H), 3.41-3.33 (m, 1H), 3.29-2.89 (m, 6H), 2.79 (s, 9H), 2.44-2.19 (m, 3H), 2.19-1.96 (m, 3H), 1.94-1.78 (m, 5H), 1.78-1.60 (m, 5H), 1.53 (d, 5H), 1.43-1.17 (m, 12H), 1.05 (dd, 6H), 0.90 (dd, 6H).

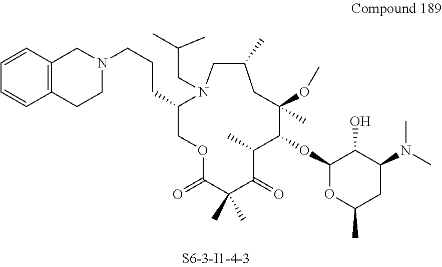

(3S,6R,8R,9R,10R)-3-(3-(3,4-dihydroisoquinolin-2(1H)-yl)propyl)-9-(((2S,3R,4S,6R)-4-(dimethylamino)-3-hydroxy-6-methyltetrahydro-2H-pyran-2-yl)oxy)-4-isobutyl-8-methoxy-6,8,10,12,12-pentamethyl-1-oxa-4-azacyclotridecane-11,13-dione (S6-3-I1-4-3)

Prepared according to the methods of S6-3-I1-1-1 from S2-1-I1-4 and 1,2,3,4-tetrahydroisoquinoline to give the title compound as a formate salt. MS (ESI+) m/z: 716.32 [M+H]+; 1H NMR (400 MHz, Methanol-d4) δ 7.41-6.89 (m, 4H), 4.66-4.40 (m, 2H), 4.27-4.01 (m, 3H), 4.01-3.62 (m, 3H), 3.62-3.30 (m, 3H), 3.15 (dd, 5H), 3.05-2.67 (m, 11H), 2.66-2.01 (m, 6H), 2.01-1.49 (m, 10H), 1.49-1.25 (m, 12H), 1.25-1.02 (m, 5H), 1.02-0.77 (m, 5H).

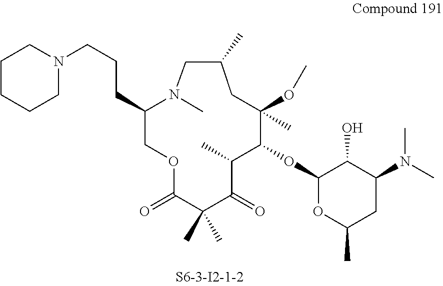

(3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(3-(piperidin-1-yl)propyl)-1-oxa-4-azacyclotridecane-11,13-dione (S6-3-I2-1-2)

Prepared according to the methods of S6-3-I1-1-1 from S2-1-I2-1 and piperidine to give the title compound as a formate salt. MS (ESI+) m/z: 626.48 [M+H]+; 1H NMR (400 MHz, Methanol-d4) δ 4.45 (d, 1H), 4.26 (t, 2H), 3.84-3.63 (m, 2H), 3.51-3.33 (m, 3H), 3.21-2.91 (m, 11H), 2.78 (s, 9H), 2.20 (s, 1H), 2.08-1.96 (m, 1H), 1.83 (p, 8H), 1.74-1.57 (m, 5H), 1.51 (q, 5H), 1.40 (d, 6H), 1.34 (t, 6H), 1.05 (d, 3H).

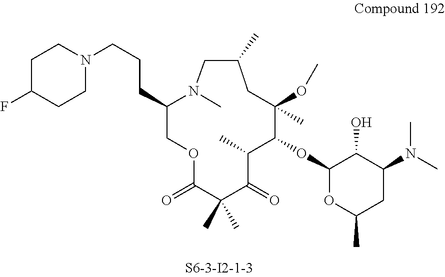

(3R,6R,8R,9R,10R)-9-(((2S,3R,4S,6R)-4-(dimethylamino)-3-hydroxy-6-methyltetrahydro-2H-pyran-2-yl)oxy)-3-(3-(4-fluoropiperidin-1-yl)propyl)-8-methoxy-4,6,8,10,12,12-hexamethyl-1-oxa-4-azacyclotridecane-11,13-dione (S6-3-I2-1-3) (Compound 23)

Prepared according to the methods of S6-3-I1-1-1 from S2-1-I2-1 and 4-fluoropiperidine to give the title compound as a formate salt. MS (ESI+) m/z: 644.36 [M+H]+; 1H NMR (400 MHz, Methanol-d4) δ 4.75 (d, 1H), 4.45 (d, 1H), 4.29 (dd, 2H), 3.90-3.64 (m, 2H), 3.54-3.32 (m, 4H), 3.07 (s, 5H), 2.95-2.59 (m, 15H), 2.24 (s, 1H), 2.00 (d, 6H), 1.78 (s, 3H), 1.69-1.46 (m, 7H), 1.46-1.25 (m, 12H), 1.07 (d, 3H).

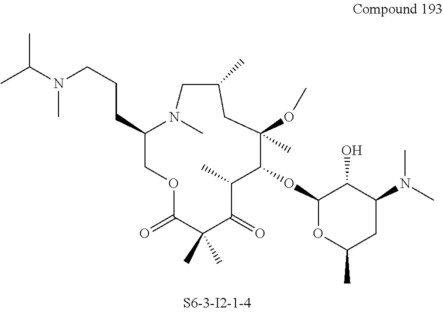

(3R,6R,8R,9R,10R)-9-(((2S,3R,4S,6R)-4-(dimethylamino)-3-hydroxy-6-methyltetrahydro-2H-pyran-2-yl)oxy)-3-(3-(isopropyl(methyl)amino)propyl)-8-methoxy-4,6,8,10,12,12-hexamethyl-1-oxa-4-azacyclotridecane-11,13-dione (S6-3-I2-1-4)

Prepared according to the methods of S6-3-I1-1-1 from S2-1-I2-1 and N-methylisopropylamine to give the title compound as a formate salt. MS (ESI+) m/z: 614.43 [M+H]+; 1H NMR (400 MHz, Methanol-d4) δ 4.71 (s, 1H), 4.45 (d, 1H), 4.27 (d, 2H), 3.87-3.63 (m, 2H), 3.53 (p, 1H), 3.49-3.33 (m, 3H), 3.05 (d, 7H), 2.77 (s, 9H), 2.70 (s, 4H), 2.17 (s, 1H), 2.01 (ddd, 1H), 1.85 (s, 3H), 1.75-1.43 (m, 7H), 1.43-1.21 (m, 18H), 1.04 (d, 3H).

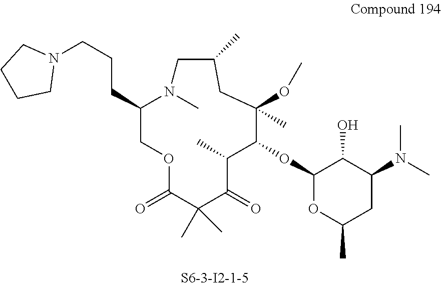 [SMILES: CO[C@]1(C)C[C@@H](C)CN(C)[C@H](CCCN2CCCC2)COC(=O)C(C)(C)C(=O)[C@H](C)[C@H]1O[C@@H]1O[C@H](C)C[C@H](N(C)C)[C@H]1O]

(3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(3-(pyrrolidin-1-yl)propyl)-1-oxa-4-azacyclotridecane-11,13-dione (S6-3-I2-1-5)

Prepared according to the methods of S6-3-I1-1-1 from S2-1-I2-1 and pyrrolidine to give the title compound as a formate salt. MS (ESI+) m/z: 612.37 [M+H]+; ¹H NMR (400 MHz, MeOD-d4): δ 4.45 (d, 1H), 4.26 (t, 2H), 3.85-3.63 (m, 2H), 3.41 (ddd, 3H), 3.31-3.21 (m, 5H), 3.15 (t, 2H), 3.05 (s, 5H), 2.78 (s, 9H), 2.19 (s, 1H), 2.13-1.97 (m, 6H), 1.86 (d, 3H), 1.60 (s, 3H), 1.54 (s, 4H), 1.45-1.23 (m, 12H), 1.05 (d, 3H).

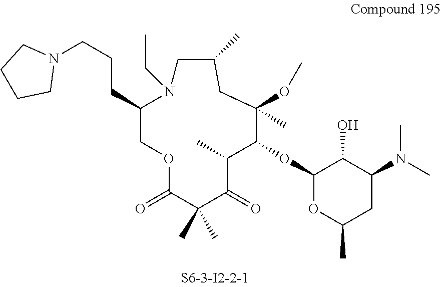

(3R,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-3-(3-(pyrrolidin-1-yl)propyl)-1-oxa-4-azacyclotridecane-11,13-dione (S6-3-I2-2-1)

Prepared according to the methods of S6-3-I1-1-1 from S2-1-I2-2 and pyrrolidine to give the title compound as a formate salt. MS (ESI+) m/z: 626.31 [M+H]+; 1H NMR (400 MHz, Methanol-d4) δ 4.46 (d, 1H), 4.13 (s, 2H), 3.82-3.49 (m, 3H), 3.49-3.20 (m, 6H), 3.21-3.04 (m, 3H), 2.92 (s, 4H), 2.77 (s, 7H), 2.17-1.94 (m, 6H), 1.94-1.68 (m, 5H), 1.68-1.41 (m, 7H), 1.43-1.26 (m, 13H), 1.21 (s, 3H), 0.97 (s, 3H).

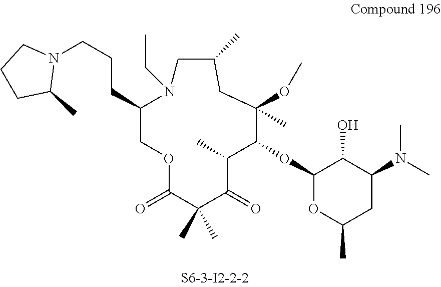

(3R,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-3-(3-((S)-2-methylpyrrolidin-1-yl)propyl)-1-oxa-4-azacyclotridecane-11,13-dione (S6-3-I2-2-2)

Prepared according to the methods of S6-3-I1-1-1 from S2-1-I2-2 and (S)-2-methylpyrrolidine to give the title compound as a formate salt. MS (ESI+) m/z: 640.32 [M+H]+; 1H NMR (400 MHz, Methanol-d4) δ 4.46 (d, 1H), 4.11 (s, 2H), 3.80-3.51 (m, 4H), 3.49-3.22 (m, 4H), 2.97 (d, 7H), 2.77 (s, 7H), 2.28 (dq, 2H), 2.14-1.92 (m, 4H), 1.92-1.65 (m, 6H), 1.66-1.26 (m, 22H), 1.19 (s, 3H), 0.95 (s, 3H).

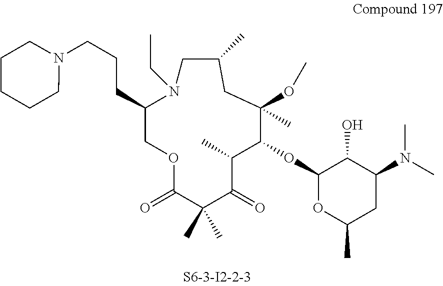

(3R,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-3-(3-(piperidin-1-yl)propyl)-1-oxa-4-azacyclotridecane-11,13-dione (S6-3-I2-2-3)

Prepared according to the methods of S6-3-I1-1-1 from S2-1-I2-2 and piperidine to give the title compound as a formate salt. MS (ESI+) m/z: 640.32 [M+H]+; 1H NMR (400 MHz, Methanol-d4) δ 4.46 (d, 1H), 4.13 (s, 2H), 3.83-3.39 (m, 4H), 3.38-3.24 (m, 2H), 3.24-2.83 (m, 10H), 2.77 (s, 7H), 2.11-1.93 (m, 2H), 1.84 (dt, 9H), 1.71-1.42 (m, 9H), 1.42-1.26 (m, 13H), 1.21 (s, 3H), 0.97 (s, 3H).

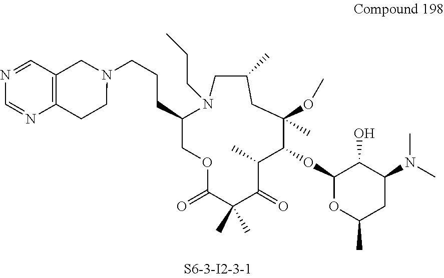

(3R,6R,8R,9R,10R)-3-(3-(7,8-dihydropyrido[4,3-d]pyrimidin-6(5H)-yl)propyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propyl-1-oxa-4-azacyclotridecane-11,13-dione (S6-3-I2-3-1)

Prepared according to the methods of S6-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and 5,6,7,8-tetrahydropyrido[4,3-d]pyrimidine to give the title compound as a formate salt. MS (ESI+) m/z: 704.04 [M+H]+; 1H NMR (400 MHz, Methanol-d4) δ 8.93 (s, 1H), 8.54 (s, 1H), 4.45 (d, 1H), 4.41-4.10 (m, 2H), 3.73 (q, 4H), 3.57-3.16 (m, 6H), 3.08-2.87 (m, 9H), 2.84 (s, 6H), 2.80-2.63 (m, 2H), 2.18-1.60 (m, 8H), 1.61-1.44 (m, 6H), 1.42-1.22 (m, 13H), 1.01-0.85 (m, 6H).

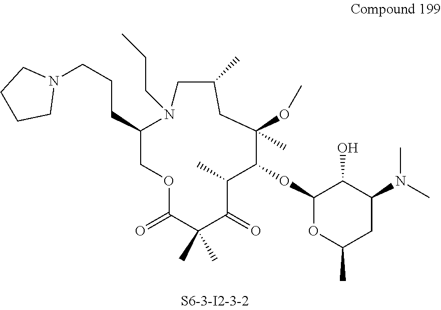

(3R,6R,8R,9R,10R)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propyl-3-(3-(pyrrolidin-1-yl)propyl)-1-oxa-4-azacyclotridecane-11,13-dione (S6-3-I2-3-2)

Prepared according to the methods of S6-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and pyrrolidine to give the title compound as a formate salt. MS (ESI+) m/z: 640.32 [M+H]+; 1H NMR (400 MHz, Methanol-d4) δ 4.45 (d, 1H), 4.09 (s, 2H), 3.79-3.64 (m, 1H), 3.57 (s, 1H), 3.44 (dd, 1H), 3.41-3.22 (m, 7H), 3.15 (qt, 3H), 2.87 (s, 4H), 2.79 (s, 8H), 2.14-1.96 (m, 6H), 1.80 (s, 4H), 1.66-1.41 (m, 8H), 1.40-1.20 (m, 13H), 0.96 (dd, 6H).

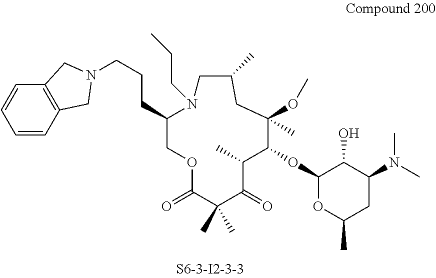

(3R,6R,8R,9R,10R)-9-(((2S,3R,4S,6R)-4-(dimethylamino)-3-hydroxy-6-methyltetrahydro-2H-pyran-2-yl)oxy)-3-(3-(isoindolin-2-yl)propyl)-8-methoxy-6,8,10,12,12-pentamethyl-4-propyl-1-oxa-4-azacyclotridecane-11,13-dione (S6-3-I2-3-3)

Prepared according to the methods of S6-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and isoindoline to give the title compound as a formate salt. MS (ESI+) m/z: 688.27 [M+H]+; 1H NMR (400 MHz, Methanol-d4) δ 7.29 (t, 4H), 4.46 (d, 1H), 4.18 (s, 6H), 3.72 (ddt, 1H), 3.61-3.31 (m, 5H), 2.98 (d, 8H), 2.79 (s, 7H), 2.02 (ddd, 2H), 1.82 (s, 7H), 1.51 (d, 5H), 1.44-1.23 (m, 13H), 1.00 (t, 6H).

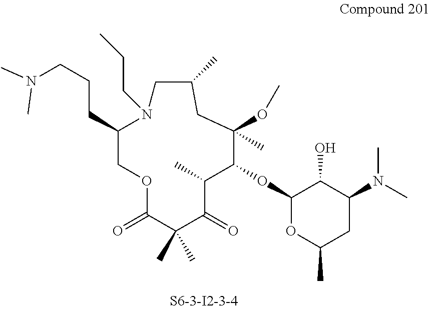

(3R,6R,8R,9R,10R)-9-(((2S,3R,4S,6R)-4-(dimethylamino)-3-hydroxy-6-methyltetrahydro-2H-pyran-2-yl)oxy)-3-(3-(dimethylamino)propyl)-8-methoxy-6,8,10,12,12-pentamethyl-4-propyl-1-oxa-4-azacyclotridecane-11,13-dione (S6-3-I2-3-4)

Prepared according to the methods of S6-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and dimethylamine to give the title compound as a formate salt. MS (ESI+) m/z: 614.26 [M+H]+; 1H NMR (400 MHz, Methanol-d4) δ 4.45 (d, 1H), 4.08 (s, 2H), 3.78-3.52 (m, 2H), 3.44 (dd, 1H), 3.38-3.32 (m, 1H), 3.14-2.52 (m, 22H), 2.21-1.93 (m, 2H), 1.75 (s, 4H), 1.64-1.40 (m, 8H), 1.39-1.18 (m, 14H), 0.96 (dd, 6H).

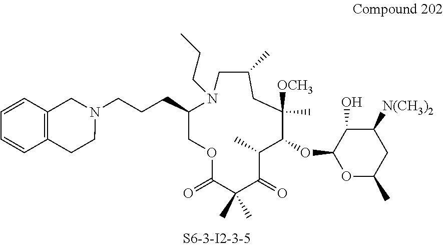

(3R,6R,8R,9R,10R)-3-(3-(3,4-Dihydroisoquinolin-2(1H)-yl)propyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propyl-1-oxa-4-azacyclotridecane-11,13-dione (S6-3-I2-3-5)

Prepared according to the methods of S6-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and 1,2,3,4-tetrahydroisoquinoline to give the title compound as a formate salt. MS (ESI+) m/z: 234.8 [M+3H]3+, 351.8 [M+2H]2+, 702.5 [M+H]+; ¹H NMR (400 MHz, Methanol-d₄) δ 6.91-6.82 (m, 1H), 6.76 (dd, 1H), 6.48 (d, 1H), 6.38 (td, 1H), 4.25 (d, 1H), 4.08 (dd, 1H), 3.87 (d, 1H), 3.77 (t, 1H), 3.55 (ddt, 1H), 3.46 (dtt, 1H), 3.16 (dt, 5H), 2.88 (d, 1H), 2.65 (d, 3H), 2.62 (s, 1H), 2.62-2.41 (m, 3H), 2.24 (s, 6H), 2.17 (d, 1H), 1.95 (dd, 1H), 1.88-1.78 (m, 2H), 1.65 (ddd, 1H), 1.55 (s, 1H), 1.54-1.47 (m, 3H), 1.41 (s, 3H), 1.39-1.31 (m, 1H), 1.23 (s, 3H), 1.21-1.09 (m, 10H), 0.93-0.77 (m, 4H), 0.72 (d, 3H).

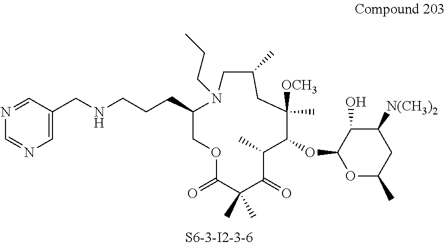

(3R,6R,8R,9R,10R)-9-(((2S,3R,4S,6R)-4-(Dimethylamino)-3-hydroxy-6-methyltetrahydro-2H-pyran-2-yl)oxy)-8-methoxy-6,8,10,12,12-pentamethyl-4-propyl-3-(3-((pyrimidin-5-ylmethyl)amino)propyl)-1-oxa-4-azacyclotridecane-11,13-dione (S6-3-I2-3-6)

Prepared according to the methods of S6-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and pyrimidin-5-ylmethanamine to give the title compound as a formate salt. MS (ESI+) m/z: 226.8 [M+3H]3+, 339.8 [M+2H]2+, 678.5 [M+H]+; ¹H NMR (400 MHz, Methanol-d₄) δ 9.16 (s, 1H), 8.90 (s, 2H), 8.49 (s, 2H), 4.47 (d, 1H), 4.24 (d, 1H), 4.12 (s, 2H), 3.53-3.37 (m, 3H), 2.98 (d, 5H), 2.84 (s, 6H), 2.06 (tq, 2H), 1.68 (s, 7H), 1.53 (s, 3H), 1.41-1.31 (m, 12H), 1.03 (t, 6H).

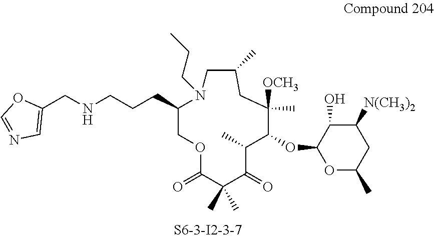

(3R,6R,8R,9R,10R)-9-(((2S,3R,4S,6R)-4-(Dimethylamino)-3-hydroxy-6-methyltetrahydro-2H-pyran-2-yl)oxy)-8-methoxy-6,8,10,12,12-pentamethyl-3-(3-((oxazol-5-ylmethyl)amino)propyl)-4-propyl-1-oxa-4-azacyclotridecane-11,13-dione (S6-3-I2-3-7)

Prepared according to the methods of S6-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and oxazol-5-ylmethanamine to give the title compound as a formate salt. MS (ESI+) m/z: 223.2 [M+3H]3+, 334.3 [M+2H]2+, 667.5 [M+H]+; ¹H NMR (400 MHz, Methanol-d₄) δ 8.46 (s, 2H), 8.25 (s, 1H), 7.18 (s, 1H), 4.47 (d, 1H), 4.24 (s, 1H), 4.09 (s, 2H), 3.75 (ddt, 1H), 3.48 (dd, 2H), 3.45-3.35 (m, 2H), 2.98 (s, 3H), 2.86 (s, 2H), 2.83 (s, 7H), 2.05 (ddd, 2H), 1.74 (s, 2H), 1.60-1.47 (m, 6H), 1.41-1.31 (m, 13H), 1.04 (d, 6H).

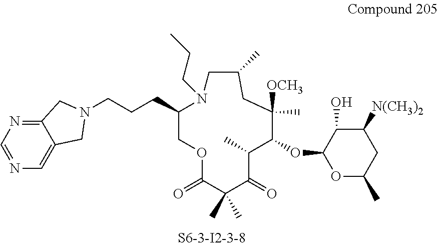

(3R,6R,8R,9R,10R)-3-(3-(5,7-Dihydro-6H-pyrrolo[3,4-d]pyrimidin-6-yl)propyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propyl-1-oxa-4-azacyclotridecane-11,13-dione (S6-3-I2-3-8): Prepared according to the methods of S6-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and 6,7-dihydro-5H-pyrrolo[3,4-d]pyrimidine to give the title compound as a formate salt. MS (ESI+) m/z: 230.8 [M+3H]3+, 345.8 [M+2H]2+, 690.5 [M+H]+; ¹H NMR (400 MHz, Methanol-d₄) δ 9.03 (s, 1H), 8.66 (s, 1H), 8.50 (s, 2H), 4.46 (d, 1H), 4.21 (dd, 2H), 4.06-3.98 (m, 3H), 3.74 (ddt, 1H), 3.51 (dd, 1H), 3.42 (ddd, 2H), 3.00 (s, 2H), 2.95 (t, 2H), 2.84 (s, 3H), 2.10-2.01 (m, 1H), 1.61-1.47 (m, 5H), 1.44-1.30 (m, 12H), 1.02 (t, 5H).

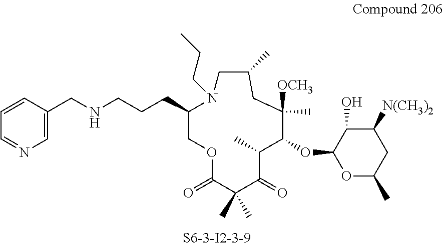

(3R,6R,8R,9R,10R)-9-(((2S,3R,4S,6R)-4-(Dimethylamino)-3-hydroxy-6-methyltetrahydro-2H-pyran-2-yl)oxy)-8-methoxy-6,8,10,12,12-pentamethyl-4-propyl-3-(3-((pyridin-3-ylmethyl)amino)propyl)-1-oxa-4-azacyclotridecane-11,13-dione (S6-3-I2-3-9)

Prepared according to the methods of S6-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and pyridin-3-ylmethanamine to give the title compound as a formate salt. MS (ESI+) m/z: 226.5 [M+3H]3+, 339.3 [M+2H]2+, 677.5 [M+H]+; ¹H NMR (400 MHz, Methanol-d₄) δ 8.67 (d, 1H), 8.59 (dd, 1H), 8.51 (s, 2H), 8.00 (dt, 1H), 7.52 (dd, 1H), 4.47 (d, 1H), 4.19 (s, 2H), 3.74 (tdd, 1H), 3.52-3.35 (m, 3H), 3.03 (q, 3H), 2.95 (s, 2H), 2.83 (s, 6H), 2.09-2.00 (m, 2H), 1.83 (s, 2H), 1.63 (s, 1H), 1.60-1.47 (m, 5H), 1.35 (dd, 12H), 1.01 (t, 5H).

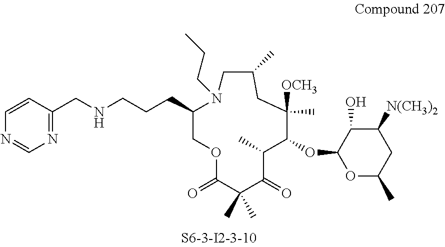

(3R,6R,8R,9R,10R)-9-(((2S,3R,4S,6R)-4-(Dimethylamino)-3-hydroxy-6-methyltetrahydro-2H-pyran-2-yl)oxy)-8-methoxy-6,8,10,12,12-pentamethyl-4-propyl-3-(3-((pyrimidin-4-ylmethyl)amino)propyl)-1-oxa-4-azacyclotridecane-11,13-dione (S6-3-I2-3-10)

Prepared according to the methods of S6-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and pyrimidin-4-ylmethanamine to give the title compound as a formate salt. MS (ESI+) m/z: 226.8 [M+3H]3+, 339.8 [M+2H]2+, 678.5 [M+H]+; ¹H NMR (400 MHz, Methanol-d₄) δ 9.19 (d, 1H), 8.79 (d, 1H), 8.51 (s, 2H), 7.59 (dd, 1H), 4.47 (d, 1H), 4.24 (s, 2H), 4.21 (s, 1H), 3.74 (ddt, 1H), 3.48 (dd, 2H), 3.40 (ddd, 2H), 3.04-2.93 (m, 5H), 2.83 (s, 6H), 2.09-2.00 (m, 2H), 1.64 (s, 1H), 1.53 (d, 5H), 1.41-1.31 (m, 12H), 1.02 (t, 5H).

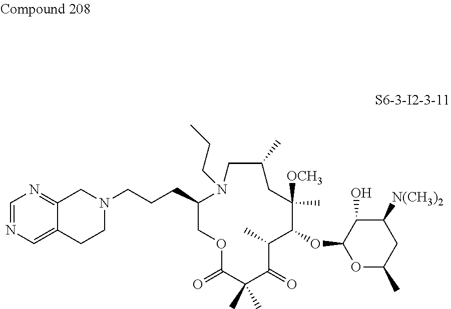

(3R,6R,8R,9R,10R)-3-(3-(5,8-dihydropyrido[3,4-d]pyrimidin-7(6H)-yl)propyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propyl-1-oxa-4-azacyclotridecane-11,13-dione (S6-3-I2-3-11)

Prepared according to the methods of S6-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and 5,6,7,8-tetrahydropyrido[3,4-d]pyrimidine to give the title compound as a formate salt. MS (ESI+) m/z: 235.5 [M+3H]3+, 352.8 [M+2H]2+, 704.5 [M+H]+; ¹H NMR (400 MHz, Methanol-d₄) δ 8.93 (s, 1H), 8.61 (s, 1H), 8.47 (s, 2H), 4.46 (d, 1H), 4.35 (s, 1H), 4.25 (d, 1H), 3.74 (s, 3H), 3.51 (dd, 1H), 3.42 (ddd, 1H), 3.29 (s, 2H), 3.00 (d, 5H), 2.92 (q, 1H), 2.85 (s, 7H), 2.74 (qd, 2H), 2.06 (ddd, 1H), 1.96 (s, 1H), 1.87-1.77 (m, 2H), 1.77-1.72 (m, 1H), 1.61-1.55 (m, 1H), 1.53 (s, 4H), 1.41-1.28 (m, 13H), 1.04-0.96 (m, 6H).

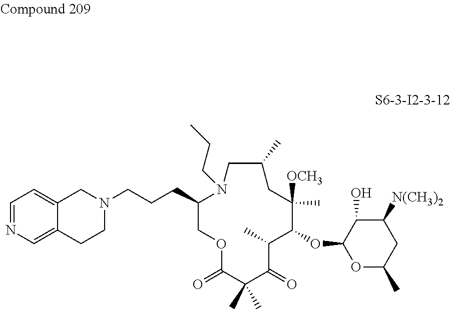

(3R,6R,8R,9R,10R)-3-(3-(3,4-dihydro-2,6-naphthyridin-2(1H)-yl)propyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propyl-1-oxa-4-azacyclotridecane-11,13-dione (S6-3-I2-3-12)

Prepared according to the methods of S6-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and 1,2,3,4-tetrahydro-2,6-naphthyridine to give the title compound as a formate salt. MS (ESI+) m/z: 235.2 [M+3H]3+, 352.3 [M+2H]2+, 703.5 [M+H]+; ¹H NMR (400 MHz, Methanol-d₄) δ 8.48 (s, 2H), 8.36-8.17 (m, 2H), 7.26 (d, 1H), 4.46 (d, 1H), 4.35 (s, 1H), 4.25 (d, 1H), 3.87-3.66 (m, 4H), 3.55-3.38 (m, 2H), 3.28 (s, 1H), 3.01 (s, 6H), 2.85 (s, 6H), 2.06 (ddd, 2H), 1.95 (s, 1H), 1.75 (s, 1H), 1.54 (s, 3H), 1.37 (dt, 14H), 1.00 (t, 3H), 0.91 (d, 3H).

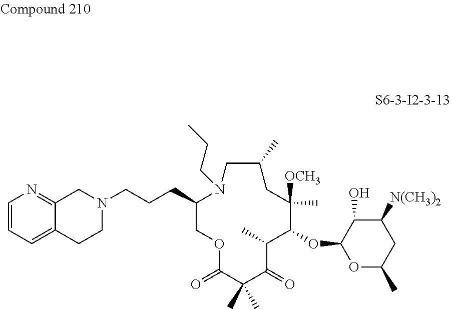

(3R,6R,8R,9R,10R)-3-(3-(5,8-Dihydro-1,7-naphthyridin-7(6H)-yl)propyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propyl-1-oxa-4-azacyclotridecane-11,13-dione (S6-3-I2-3-13)

Prepared according to the methods of S6-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and 5,6,7,8-tetrahydro-1,7-naphthyridine to give the title compound as a formate salt. MS (ESI+) m/z: 235.2 [M+3H]3+, 352.3 [M+2H]2+, 703.5 [M+H]+; ¹H NMR (400 MHz, Methanol-d₄) δ 8.46 (s, 2H), 8.36 (dd, 1H), 7.62 (dd, 1H), 7.27 (dd, 1H), 4.46 (d, 1H), 4.36 (s, 1H), 4.27 (d, 1H), 3.86-3.75 (m, 3H), 3.75-3.71 (m, 1H), 3.55-3.39 (m, 2H), 3.11-2.97 (m, 8H), 2.92 (d, 1H), 2.85 (s, 6H), 2.77 (t, 2H), 2.06 (dt, 1H), 1.95 (s, 1H), 1.84 (dt, 2H), 1.77 (s, 1H), 1.62-1.49 (m, 6H), 1.42-1.31 (m, 13H), 1.00 (t, 3H), 0.92 (d, 3H).

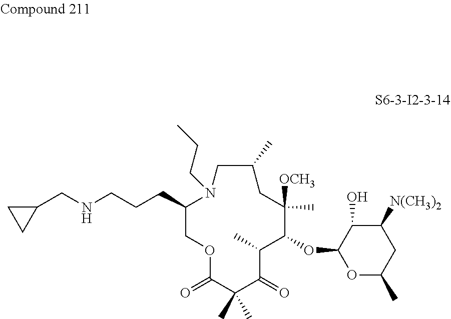

(3R,6R,8R,9R,10R)-3-(3-((cyclopropylmethyl)amino)propyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propyl-1-oxa-4-azacyclotridecane-11,13-dione (S6-3-I2-3-14)

Prepared according to the methods of S6-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and cyclopropylmethanamine to give the title compound as a formate salt. MS (ESI+) m/z: 214.2 [M+3H]3+, 320.8 [M+2H]2+, 640.5 [M+H]+; ¹H NMR (400 MHz, Methanol-d₄) δ 8.53 (s, 2H), 4.47 (d, 1H), 4.17 (s, 1H), 3.73 (ddd, 1H), 3.55-3.43 (m, 2H), 3.39 (td, 1H), 3.12 (dt, 2H), 2.93 (s, 2H), 2.82 (s, 6H), 2.80 (s, 1H), 2.66 (q, 1H), 2.09-2.00 (m, 1H), 1.81 (s, 2H), 1.62 (s, 1H), 1.54 (s, 3H), 1.53-1.46 (m, 1H), 1.42-1.32 (m, 11H), 1.30 (s, 1H), 1.00 (q, 5H), 0.85 (d, 3H).

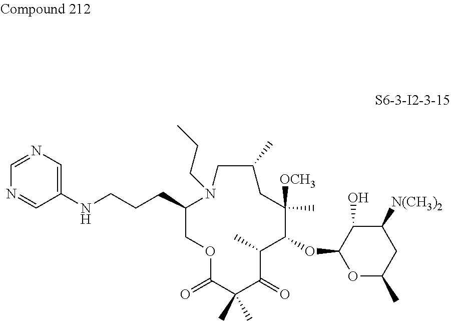

(3R,6R,8R,9R,10R)-9-(((2S,3R,4S,6R)-4-(Dimethylamino)-3-hydroxy-6-methyltetrahydro-2H-pyran-2-yl)oxy)-8-methoxy-6,8,10,12,12-pentamethyl-4-propyl-3-(3-(pyrimidin-5-ylamino)propyl)-1-oxa-4-azacyclotridecane-11,13-dione (S6-3-I2-3-15)

Prepared according to the methods of S6-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and pyrimidin-5-amine to give the title compound as a formate salt. MS (ESI+) m/z: 222.2 [M+3H]3+, 332.8 [M+2H]2+, 664.5 [M+H]+; ¹H NMR (400 MHz, Methanol-d₄) δ 8.54 (s, 1H), 8.40 (s, 1H), 8.16 (s, 2H), 4.46 (d, 1H), 3.72 (tq, 1H), 3.47 (dd, 1H), 3.26 (s, 2H), 2.90 (s, 1H), 2.79 (s, 6H), 2.02 (ddd, 1H), 1.77 (s, 1H), 1.58-1.45 (m, 5H), 1.41-1.30 (m, 11H), 0.98 (s, 2H).

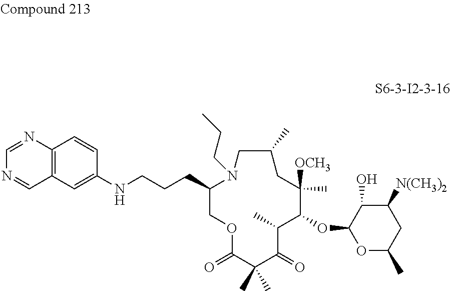

(3R,6R,8R,9R,10R)-9-(((2S,3R,4S,6R)-4-(Dimethylamino)-3-hydroxy-6-methyltetrahydro-2H-pyran-2-yl)oxy)-8-methoxy-6,8,10,12,12-pentamethyl-4-propyl-3-(3-(quinazolin-6-ylamino)propyl)-1-oxa-4-azacyclotridecane-11,13-dione (S6-3-I2-3-16)

Prepared according to the methods of S6-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and quinazolin-6-amine to give the title compound as a formate salt. MS (ESI+) m/z: 238.8 [M+3H]3+, 357.8 [M+2H]2+, 714.5 [M+H]+; ¹H NMR (400 MHz, Methanol-d₄) δ 9.09 (s, 1H), 8.75 (s, 1H), 8.45 (s, 1H), 7.64 (d, 1H), 7.39 (dd, 1H), 6.78 (d, 1H), 4.30 (d, 1H), 4.18 (d, 1H), 3.86 (d, 1H), 3.78 (t, 1H), 3.52 (ddt, 1H), 3.30-3.23 (m, 2H), 3.17 (s, 1H), 2.98 (s, 1H), 2.86 (s, 1H), 2.67 (s, 2H), 2.45 (s, 4H), 2.42 (s, 3H), 1.86 (t, 1H), 1.76 (d, 1H), 1.64 (s, 2H), 1.50 (s, 1H), 1.41 (s, 3H), 1.37-1.25 (m, 4H), 1.25-1.10 (m, 13H), 0.84-0.66 (m, 4H), 0.60 (d, 2H).

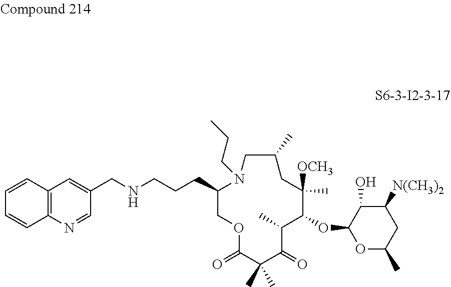

(3R,6R,8R,9R,10R)-9-(((2S,3R,4S,6R)-4-(Dimethylamino)-3-hydroxy-6-methyltetrahydro-2H-pyran-2-yl)oxy)-8-methoxy-6,8,10,12,12-pentamethyl-4-propyl-3-(3-((quinolin-3-ylmethyl)amino)propyl)-1-oxa-4-azacyclotridecane-11,13-dione (S6-3-I2-3-17)

Prepared according to the methods of S6-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and quinolin-3-ylmethanamine to give the title compound as a formate salt. MS (ESI+) m/z: 243.2 [M+3H]3+, 364.3 [M+2H]2+, 727.5 [M+H]+; ¹H NMR (400 MHz, Methanol-d₄) δ 8.95 (d, 1H), 8.53 (s, 2H), 8.48 (d, 1H), 8.08 (d, 1H), 8.00 (dd, 1H), 7.84 (ddd, 1H), 7.69 (ddd, 1H), 4.46 (d, 1H), 4.31 (s, 1H), 4.14 (s, 1H), 3.77-3.68 (m, 1H), 3.51-3.34 (m, 2H), 3.01 (q, 3H), 2.91 (s, 2H), 2.81 (s, 6H), 2.03 (ddd, 1H), 1.83-1.77 (m, 2H), 1.59-1.45 (m, 5H), 1.39-1.29 (m, 11H), 0.99 (d, 2H), 0.96 (s, 2H).

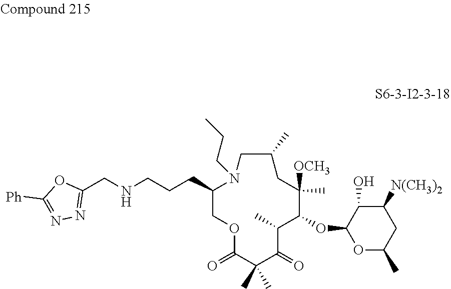

(3R,6R,8R,9R,10R)-9-(((2S,3R,4S,6R)-4-(Dimethylamino)-3-hydroxy-6-methyltetrahydro-2H-pyran-2-yl)oxy)-8-methoxy-6,8,10,12,12-pentamethyl-3-(3-(((5-phenyl-1,3,4-oxadiazol-2-yl)methyl)amino)propyl)-4-propyl-1-oxa-4-azacyclotridecane-11,13-dione (S6-3-I2-3-18)

Prepared according to the methods of 56-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and (5-phenyl-1,3,4-oxadiazol-2-yl)methanamine to give the title compound as a formate salt. MS (ESI+) m/z: 248.9 [M+3H]3+, 372.8 [M+2H]2+, 744.5 [M+H]+; ¹H NMR (400 MHz, Methanol-d₄) δ 8.53 (s, 1H), 8.08 (ddd, 2H), 7.68-7.54 (m, 3H), 4.46 (d, 1H), 4.15 (s, 2H), 3.78-3.68 (m, 1H), 3.51-3.42 (m, 2H), 3.42-3.33 (m, 1H), 2.97 (s, 2H), 2.80 (s, 6H), 2.03 (ddd, 1H), 1.68 (s, 2H), 1.59-1.45 (m, 5H), 1.40-1.33 (m, 9H), 1.31 (s, 2H), 1.01 (s, 3H).

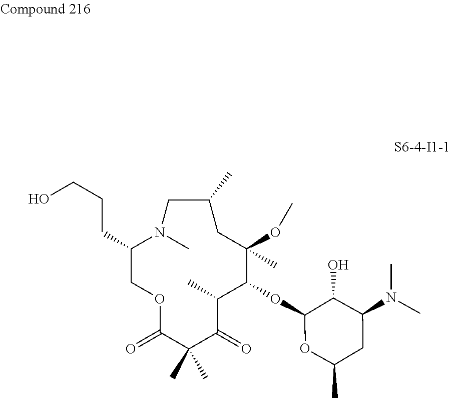

(3S,6R,8R,9R,10R)-9-(((2S,3R,4S,6R)-4-(dimethylamino)-3-hydroxy-6-methyltetrahydro-2H-pyran-2-yl)oxy)-3-(3-hydroxypropyl)-8-methoxy-4,6,8,10,12,12-hexamethyl-1-oxa-4-azacyclotridecane-11,13-dione (S6-4-I1-1)

Prepared from S6-1-I1-1 according to the methods of S2-2-13-1 to give the title compound as a formate salt. MS (ESI+) m/z: 559.35 [M+H]+; 1H NMR (400 MHz, Methanol-d4) δ 4.44 (d, 1H), 4.24 (t, 2H), 3.86-3.67 (m, 2H), 3.64 (t, 2H), 3.49-3.37 (m, 2H), 3.33 (d, 3H), 3.04 (d, 7H), 2.82 (dd, 1H), 2.75 (s, 6H), 2.21 (s, 1H), 2.07-1.90 (m, 2H), 1.89-1.54 (m, 4H), 1.49 (d, 5H), 1.45-1.24 (m, 13H), 1.06 (d, 3H).

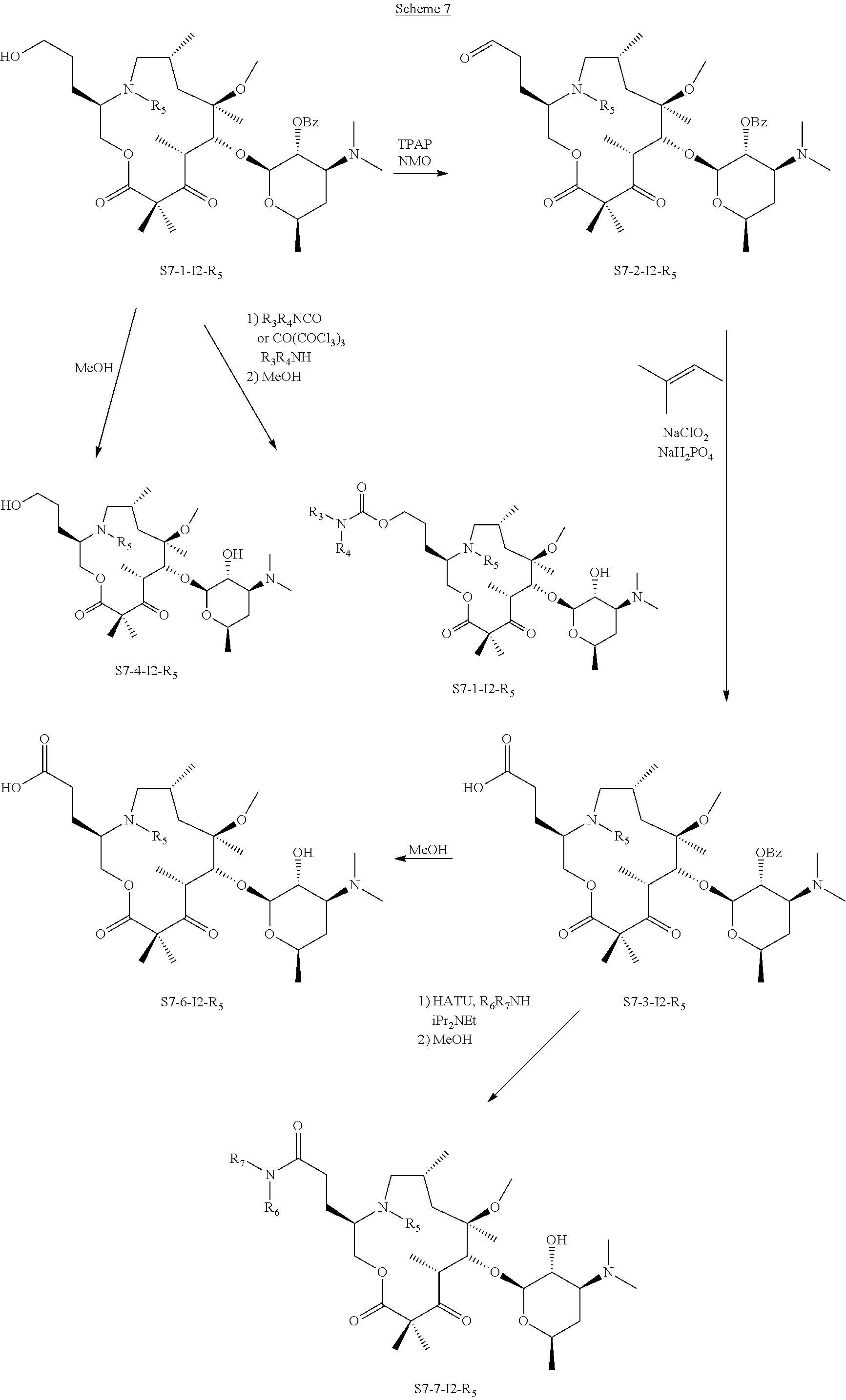

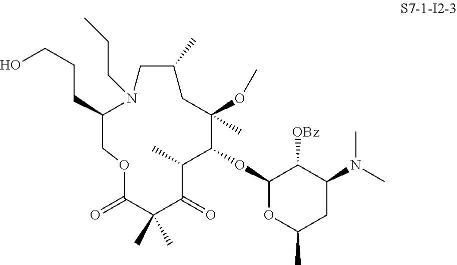

(2S,3R,4S,6R)-4-(dimethylamino)-2-(((3R,6R,8R,9R,10R)-3-(3-hydroxypropyl)-8-methoxy-6,8,10,12,12-pentamethyl-11,13-dioxo-4-propyl-1-oxa-4-azacyclotridecan-9-yl)oxy)-6-methyltetrahydro-2H-pyran-3-yl benzoate (S7-1-12-3): Prepared according to the methods of S6-1-I1-1, substituting intermediate 12 and propanal to give S7-1-12-3 as a formate salt. MS (ESI+) m/z: 346.3 [M+2H]²⁺, 691.5 [M+H]⁺.

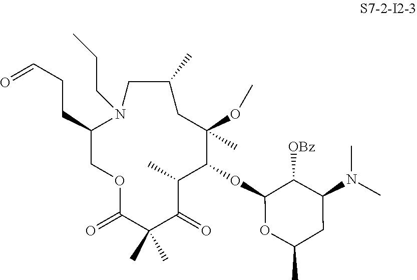

(2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate (S7-2-I2-3): Prepared according to the methods of S6-2-I1-1, substituting intermediate 12 and propanal to give S7-2-I2-3. MS (ESI+) m/z: 345.3 [M+2H]²⁺, 689.5 [M+H]⁺.

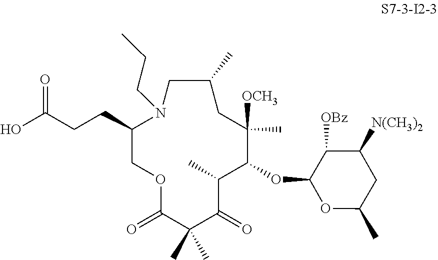

3-((3R,6R,8R,9R,10R)-9-(((2S,3R,4S,6R)-3-(Benzoyloxy)-4-(dimethylamino)-6-methyltetrahydro-2H-pyran-2-yl)oxy)-8-methoxy-6,8,10,12,12-pentamethyl-11,13-dioxo-4-propyl-1-oxa-4-azacyclotridecan-3-yl)propanoic acid (S7-3-I2-3)

In a 40 mL vial was a solution of aldehyde S7-2-I2-3 (357 mg, 0.52 mmol) and 2-methyl-2-butene (1.36 mL, 12.9 mmol) in 5 mL of tBuOH at rt. A solution of NaH2PO4 (704 mg, 5.18 mmol) and sodium chlorite (175 mg, 1.55 mmol) in 5 mL of water was added and the biphasic mixture was stirred vigourously at room temperature. After 30 min, UPLC indicated complete conversion to the desire mass. The mixture was diluted with EtOAc and poured into satd sodium sulfite and stirred for 15 minutes. The aqueous phase was saturated by adding NaCl solid and extracted with EtOAc (3×), then the combined organic phases were washed with brine, dried over Na2SO4, filtered and concentrated to give S7-3-I2-3. The material was used in the next step without further purification. MS (ESI+) m/z: 353.2 [M+2H]2+, 705.4 [M+H]+.

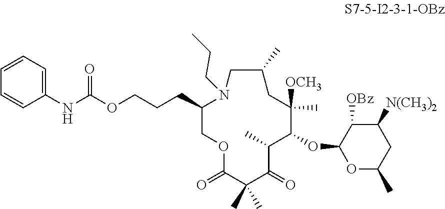

(2S,3R,4S,6R)-4-(Dimethylamino)-2-(((3R,6R,8R,9R,10R)-8-methoxy-6,8,10,12,12-pentamethyl-11,13-dioxo-3-(3-((phenylcarbamoyl)oxy)propyl)-4-propyl-1-oxa-4-azacyclotridecan-9-yl)oxy)-6-methyltetrahydro-2H-pyran-3-yl benzoate (S6-4-I2-3-1-OBz)

The alcohol S7-1-I2-3 (40 mg, 0.058 mmol) was dissolved in THE (1 mL). Phenyl isocyanate (0.015 mL, 0.14 mmol) was slowly added dropwise. The reaction mixture was heated at 70° C. for 3 days. The mixture was concentrated in vacuo. The residue was purified on 4 g of silica gel (elution with 0-10% MeOH-dichloromethane+0.5% of 30% aq NH4OH) to give the S7-5-I2-3-1-OBz (45 mg, 96%). MS (ESI+) m/z: 405.8 [M+2H]2+, 810.5 [M+H]+.

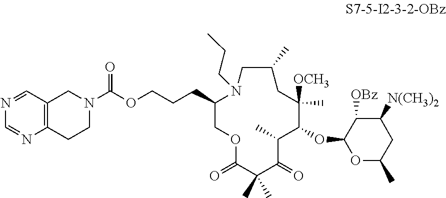

3-((3R,6R,8R,9R,10R)-9-(((2S,3R,4S,6R)-3-(Benzoyloxy)-4-(dimethylamino)-6-methyltetrahydro-2H-pyran-2-yl)oxy)-8-methoxy-6,8,10,12,12-pentamethyl-11,13-dioxo-4-propyl-1-oxa-4-azacyclotridecan-3-yl)propyl 7,8-dihydropyrido[4,3-d]pyrimidine-6(5H)-carboxylate (S7-5-I2-3-2-OBz)

The alcohol S7-1-12-3 (35 mg, 0.051 mmol) was dissolved in DCM (1 mL). Triphosgene (1.5 equiv, 22.5 mg, 0.076 mmol) was added. After 1 h at rt, UPLC shows the intermediate formed. The amine (11.0 mg, 0.010 mmol) was added and the reaction was heated at 40° C. for 1 h. UPLC shows the desired product appeared. The reaction mixture was diluted with DCM and water, extracted with DCM and dried over Na2SO4. The mixture was purified on 4 g of silica gel (elution with 0-10% MeOH-dichloromethane+0.5% of 30% aq NH4OH) to give S7-5-I2-3-2-OBz (30 mg, 70%). MS (ESI+) m/z: 426.8 [M+2H]²⁺, 852.5 [M+H]⁺.

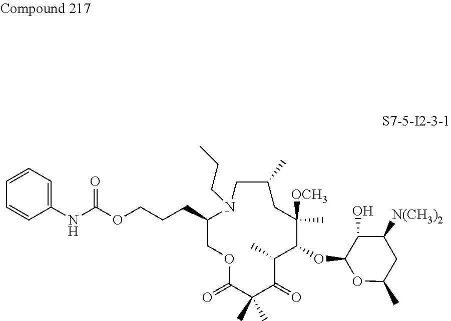

3-((3R,6R,8R,9R,10R)-9-(((2S,3R,4S,6R)-4-(Dimethylamino)-3-hydroxy-6-methyltetrahydro-2H-pyran-2-yl)oxy)-8-methoxy-6,8,10,12,12-pentamethyl-11,13-dioxo-4-propyl-1-oxa-4-azacyclotridecan-3-yl)propyl phenylcarbamate (S6-4-I2-3-1)

Compound S6-4-I2-3-1-OBz (45 mg, 0.056 mmol) was dissolved in MeOH (1 mL), and the reaction mixture was heated to 40° C. (external temp.) overnight. The reaction mixture was cooled to rt and was concentrated under reduced pressure. The material was purified by HPLC (Atlantis T3 column, 5-30% MeCN-water-0.1% HCO2H) to give 6.52 mg of S6-4-I2-3-1 as a formate salt. MS (ESI+) m/z: 236.2 [M+3H]3+, 353.8 [M+2H]2+, 706.5 [M+H]+; ¹H NMR (400 MHz, Methanol-d₄) δ 7.32 (d, 2H), 7.16 (t, 2H), 6.91 (t, 1H), 4.28 (d, 1H), 4.15-4.01 (m, 3H), 3.87 (d, 1H), 3.79 (t, 1H), 3.58 (d, 1H), 3.55-3.44 (m, 1H), 3.21 (d, 3H), 2.93 (s, 1H), 2.74 (t, 1H), 2.68 (s, 2H), 2.55 (d, 1H), 2.48 (d, 2H), 2.36 (s, 5H), 2.22-2.15 (m, 1H), 1.97 (t, 1H), 1.76-1.67 (m, 1H), 1.67-1.54 (m, 4H), 1.42 (s, 3H), 1.36 (dd, 3H), 1.24 (s, 4H), 1.22-1.12 (m, 10H), 0.91 (d, 1H), 0.82 (t, 3H), 0.73 (d, 3H).

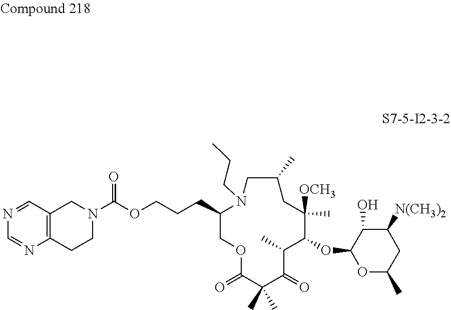

3-((3R,6R,8R,9R,10R)-9-(((2S,3R,4S,6R)-4-(Dimethylamino)-3-hydroxy-6-methyltetrahydro-2H-pyran-2-yl)oxy)-8-methoxy-6,8,10,12,12-pentamethyl-11,13-dioxo-4-propyl-1-oxa-4-azacyclotridecan-3-yl)propyl 7,8-dihydropyrido[4,3-d]pyrimidine-6(5H)-carboxylate (S7-5-I2-3-2)

Compound S7-5-I2-3-2-OBz (30 mg, 0.035 mmol) was dissolved in MeOH (1 mL), and the reaction mixture was heated to 40° C. (external temp.) overnight. The reaction mixture was cooled to rt and was concentrated under reduced pressure. The material was purified by HPLC (Atlantis T3 column, 5-30% MeCN-water-0.1% HCO2H) to give 2.35 mg of the title compound as a formate salt. MS (ESI+) m/z: 236.2 [M+3H]3+, 353.8 [M+2H]2+, 706.5 [M+H]+; ¹H NMR (400 MHz, Methanol-d₄) δ 8.93 (s, 1H), 8.60 (s, 1H), 8.52 (s, 1H), 4.44 (d, 1H), 4.19 (s, 1H), 3.85 (s, 2H), 3.69 (s, 1H), 3.41 (s, 1H), 2.98 (t, 3H), 2.74 (s, 3H), 1.97 (d, 2H), 1.72 (s, 1H), 1.51 (s, 3H), 1.46 (s, 2H), 1.40-1.28 (m, 12H), 0.92 (s, 2H), 0.81 (s, 1H).

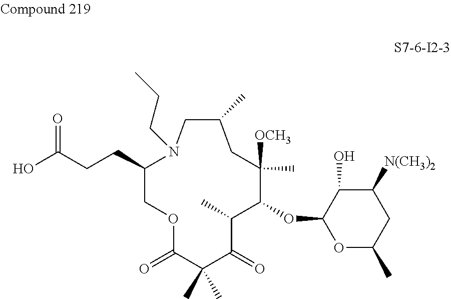

3-((3R,6R,8R,9R,10R)-9-(((2S,3R,4S,6R)-4-(Dimethylamino)-3-hydroxy-6-methyltetrahydro-2H-pyran-2-yl)oxy)-8-methoxy-6,8,10,12,12-pentamethyl-11,13-dioxo-4-propyl-1-oxa-4-azacyclotridecan-3-yl)propanoic acid (S7-6-I2-3)

Compound S7-3-12-3 (17 mg, 0.024 mmol) was dissolved in MeOH (1 mL), and the reaction mixture was heated to 40° C. (external temp.) overnight. The reaction mixture was cooled to rt and was concentrated under reduced pressure. The material was purified by HPLC (Atlantis T3 column, 5-30% MeCN-water-0.1% HCO2H) to give 4.56 mg of S7-6-I2-3 as a formate salt. MS (ESI+) m/z: 301.2 [M+2H]2+, 601.4 [M+H]+; ¹H NMR (400 MHz, Methanol-d₄) δ 8.51 (s, 1H), 4.45 (d, 1H), 4.34 (d, 1H), 4.27 (d, 1H), 3.74 (ddd, 2H), 3.51-3.33 (m, 4H), 3.02 (s, 3H), 2.81 (s, 6H), 2.45 (dt, 1H), 2.33 (ddd, 1H), 2.14 (s, 1H), 2.03 (ddd, 1H), 1.67 (s, 1H), 1.52 (s, 4H), 1.42-1.31 (m, 11H), 1.05 (t, 6H).

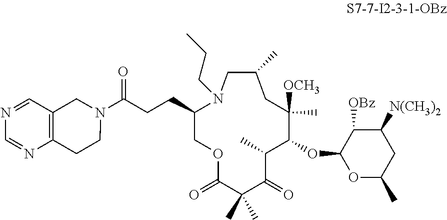

(2S,3R,4S,6R)-2-(((3R,6R,8R,9R,10R)-3-(3-(7,8-Dihydropyrido[4,3-d]pyrimidin-6(5H)-yl)-3-oxopropyl)-8-methoxy-6,8,10,12,12-pentamethyl-11,13-dioxo-4-propyl-1-oxa-4-azacyclotridecan-9-yl)oxy)-4-(dimethylamino)-6-methyltetrahydro-2H-pyran-3-yl benzoate (S7-7-I2-3-1-OBz)

In a 4 mL vial was a solution of S7-3-12-3 (30 mg, 0.043 mmol) and 5,6,7,8-tetrahydropyrido[4,3-d]pyrimidine (8.6 mg, 0.064 mmol) in 1.2 mL of DCM at rt. DIEA (0.018 mL, 0.11 mmol) was added followed by HATU (17.7 mg, 0.047 mmol) and the resulting mixture was stirred at rt for 16 h. UPLC shows full conversion. The reaction mixture was partitioned between MTBE and satd aq NaHCO3. The aqueous phase was extracted twice with ethyl acetate and the combined organic phases were dried over Na2SO4, filtered and concentrated. The mixture was purified on 4 g of silica gel (elution with 0-10% MeOH-dichloromethane+0.5% of 30% aq NH4OH) to give S7-7-I2-3-1-OBz (22 mg, 63%). MS (ESI+) m/z: 411.8 [M+2H]²⁺, 822.5 [M+H]⁺.

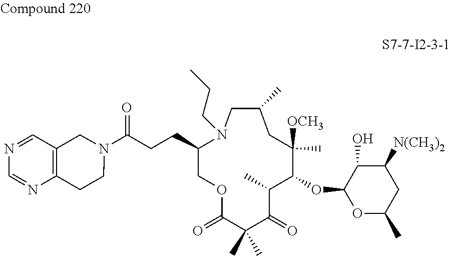

(3R,6R,8R,9R,10R)-3-(3-(7,8-Dihydropyrido[4,3-d]pyrimidin-6(5H)-yl)-3-oxopropyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propyl-1-oxa-4-azacyclotridecane-11,13-dione (S7-7-I2-3-1)

Compound S7-7-I2-3-1-OBz (22 mg, 0.027 mmol) was dissolved in MeOH (1 mL), and the reaction mixture was heated to 40° C. (external temp.) overnight. The reaction mixture was cooled to rt and was concentrated under reduced pressure. The material was purified by HPLC (Atlantis T3 column, 5-30% MeCN-water-0.1% HCO2H) to give 6.67 mg of S7-7-I2-3-1 as a formate salt. MS (ESI+) m/z: 359.8 [M+2H]2+, 718.5 [M+H]+; ¹H NMR (400 MHz, Methanol-d₄) δ 8.96 (d, 1H), 8.63 (d, 1H), 8.54 (s, 1H), 4.48-4.41 (m, 1H), 3.97 (s, 1H), 3.72 (s, 1H), 3.45 (ddd, 1H), 3.28 (s, 1H), 2.99 (t, 2H), 2.91 (s, 1H), 2.77 (s, 5H), 2.01 (dt, 1H), 1.75 (s, 1H), 1.52 (dd, 2H), 1.47 (s, 2H), 1.39-1.21 (m, 11H), 0.99 (s, 2H).

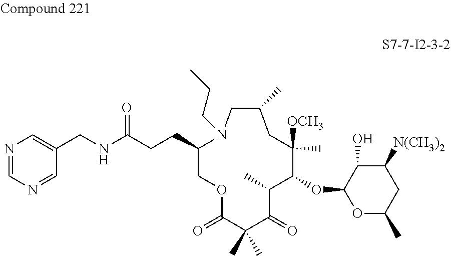

3-((3R,6R,8R,9R,10R)-9-(((2S,3R,4S,6R)-4-(Dimethylamino)-3-hydroxy-6-methyltetrahydro-2H-pyran-2-yl)oxy)-8-methoxy-6,8,10,12,12-pentamethyl-11,13-dioxo-4-propyl-1-oxa-4-azacyclotridecan-3-yl)-N-(pyrimidin-5-ylmethyl)propanamide (S7-7-I2-3-2)

Prepared according to the methods of S7-7-I2-3-1, substituting pyrimidin-5-ylmethanamine to give S7-7-I2-3-2 as a formate salt. MS (ESI+) m/z: 346.8 [M+2H]2+, 692.5 [M+H]+; ¹H NMR (400 MHz, Methanol-d₄) δ 9.07 (s, 1H), 8.78 (s, 2H), 8.49 (s, 1H), 4.50-4.39 (m, 2H), 3.73 (ddd, 1H), 3.47 (dd, 1H), 3.38 (ddd, 2H), 3.26 (s, 1H), 2.95 (s, 1H), 2.80 (s, 5H), 2.45 (s, 1H), 2.02 (ddd, 1H), 1.50 (s, 3H), 1.38-1.28 (m, 10H), 0.98 (s, 2H).

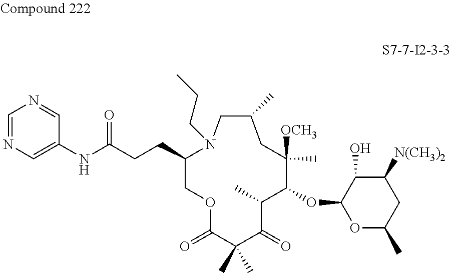

3-((3R,6R,8R,9R,10R)-9-(((2S,3R,4S,6R)-4-(Dimethylamino)-3-hydroxy-6-methyltetrahydro-2H-pyran-2-yl)oxy)-8-methoxy-6,8,10,12,12-pentamethyl-11,13-dioxo-4-propyl-1-oxa-4-azacyclotridecan-3-yl)-N-(pyrimidin-5-yl)propanamide (S7-7-I2-3-3)

Prepared according to the methods of S7-7-I2-3-1, substituting pyrimidin-5-amine to give S7-7-I2-3-3 as a formate salt. MS (ESI+) m/z: 339.7 [M+2H]2+, 678.4 [M+H]+; ¹H NMR (400 MHz, Methanol-d₄) δ 9.03 (s, 2H), 8.86 (s, 1H), 8.52 (s, 1H), 4.43 (d, 1H), 3.97 (s, 1H), 3.73-3.65 (m, 1H), 3.48-3.39 (m, 1H), 2.82 (s, 1H), 2.75 (s, 5H), 1.98 (d, 1H), 1.69 (s, 1H), 1.49 (s, 5H), 1.36 (s, 3H), 1.29 (dd, 8H), 0.95 (s, 2H), 0.84 (s, 1H).

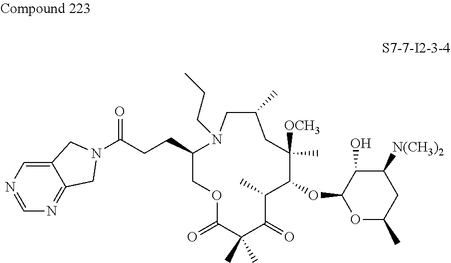

(3R,6R,8R,9R,10R)-3-(3-(5,7-Dihydro-6H-pyrrolo[3,4-d]pyrimidin-6-yl)-3-oxopropyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propyl-1-oxa-4-azacyclotridecane-11,13-dione (S7-7-I2-3-4)

Prepared according to the methods of S7-7-I2-3-1, substituting 6,7-dihydro-5H-pyrrolo[3,4-d]pyrimidine to give S7-7-I2-3-4 as a formate salt. MS (ESI+) m/z: 352.8 [M+2H]2+, 704.5 [M+H]+; ¹H NMR (400 MHz, Methanol-d₄) δ 9.09 (s, 1H), 8.78 (d, 1H), 8.51 (s, 1H), 5.08-4.98 (m, 1H), 4.98-4.92 (m, 1H), 4.44 (d, 1H), 3.76-3.67 (m, 1H), 3.44 (dd, 2H), 3.39-3.31 (m, 3H), 2.95 (s, 1H), 2.78 (s, 5H), 2.73 (s, 1H), 2.64 (s, 1H), 2.01 (ddd, 1H), 1.79 (s, 1H), 1.62 (s, 1H), 1.53 (d, 1H), 1.49 (s, 3H), 1.32 (td, 11H), 1.01 (s, 2H).

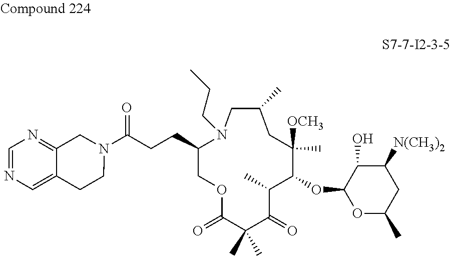

(3R,6R,8R,9R,10R)-3-(3-(5,8-Dihydropyrido[3,4-d]pyrimidin-7(6H)-yl)-3-oxopropyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propyl-1-oxa-4-azacyclotridecane-11,13-dione (S7-7-I2-3-5)

Prepared according to the methods of S7-7-I2-3-1, substituting 5,6,7,8-tetrahydropyrido[3,4-d]pyrimidine to give S7-7-I2-3-5 as a formate salt. MS (ESI+) m/z: 359.8 [M+2H]2+, 718.5 [M+H]+; ¹H NMR (400 MHz, Methanol-d₄) δ 8.95 (d, 1H), 8.61 (d, 1H), 8.53 (s, 1H), 4.41 (s, 1H), 3.89 (dt, 3H), 3.69-3.63 (m, 2H), 3.40 (s, 1H), 2.79 (s, 2H), 2.70 (s, 4H), 2.19 (s, 1H), 1.94 (d, 2H), 1.66 (s, 1H), 1.48 (s, 4H), 1.36-1.22 (m, 15H), 0.92 (s, 2H), 0.81 (s, 1H).

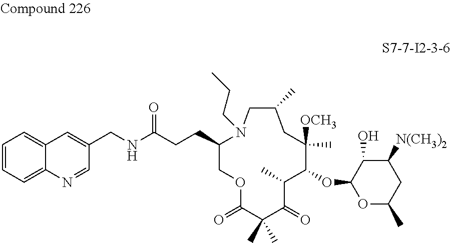

3-((3R,6R,8R,9R,10R)-9-(((2S,3R,4S,6R)-4-(Dimethylamino)-3-hydroxy-6-methyltetrahydro-2H-pyran-2-yl)oxy)-8-methoxy-6,8,10,12,12-pentamethyl-11,13-dioxo-4-propyl-1-oxa-4-azacyclotridecan-3-yl)-N-(quinolin-3-ylmethyl)propanamide (S7-7-I2-3-6)

Prepared according to the methods of S7-7-I2-3-1, substituting quinolin-3-ylmethanamine to give the title compound as a formate salt. MS (ESI+) m/z: 371.3 [M+2H]2+, 741.5 [M+H]+; ¹H NMR (400 MHz, Methanol-d₄) δ 8.83 (d, 1H), 8.53 (s, 1H), 8.30-8.25 (m, 1H), 8.02 (dd, 1H), 7.94 (dd, 1H), 7.76 (ddd, 1H), 7.62 (ddd, 1H), 4.60 (s, 2H), 4.43 (d, 1H), 3.68 (td, 1H), 3.42 (dd, 1H), 3.27 (s, 1H), 2.80 (s, 1H), 2.73 (s, 5H), 2.55 (s, 1H), 2.35 (s, 1H), 1.97 (ddd, 2H), 1.48 (s, 3H), 1.44 (d, 2H), 1.33 (s, 3H), 1.28 (dd, 9H), 0.87 (s, 2H).

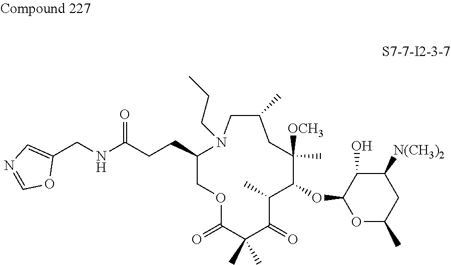

3-((3R,6R,8R,9R,10R)-9-(((2S,3R,4S,6R)-4-(Dimethylamino)-3-hydroxy-6-methyltetrahydro-2H-pyran-2-yl)oxy)-8-methoxy-6,8,10,12,12-pentamethyl-11,13-dioxo-4-propyl-1-oxa-4-azacyclotridecan-3-yl)-N-(oxazol-5-ylmethyl)propanamide (S7-7-I2-3-7)

Prepared according to the methods of S7-7-I2-3-1, substituting oxazol-5-ylmethanamine to give S7-7-I2-3-7 as a formate salt. MS (ESI+) m/z: 341.2 [M+2H]2+, 681.4 [M+H]+; ¹H NMR (400 MHz, Methanol-d₄) δ 8.50 (s, 2H), 8.16 (s, 1H), 7.03 (s, 1H), 4.51 (d, 1H), 4.48-4.38 (m, 3H), 3.76-3.68 (m, 1H), 3.47 (dd, 2H), 3.42-3.31 (m, 3H), 2.92 (s, 1H), 2.80 (s, 7H), 2.75 (s, 1H), 2.39 (s, 1H), 2.02 (ddd, 2H), 1.58-1.51 (m, 2H), 1.50 (s, 4H), 1.40-1.28 (m, 14H), 0.98 (s, 3H).

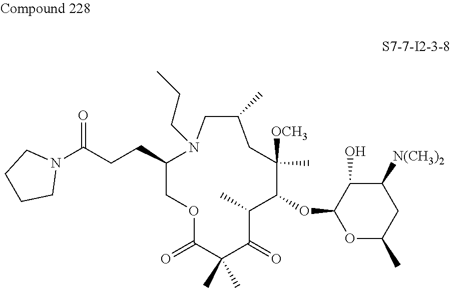

(3R,6R,8R,9R,10R)-9-(((2S,3R,4S,6R)-4-(Dimethylamino)-3-hydroxy-6-methyltetrahydro-2H-pyran-2-yl)oxy)-8-methoxy-6,8,10,12,12-pentamethyl-3-(3-oxo-3-(pyrrolidin-1-yl)propyl)-4-propyl-1-oxa-4-azacyclotridecane-11,13-dione (S7-7-I2-3-8)

Prepared according to the methods of S7-7-I2-3-1, substituting pyrrolidine to give S7-7-I2-3-8 as a formate salt. MS (ESI+) m/z: 327.8 [M+2H]2+, 654.5 [M+H]+; ¹H NMR (400 MHz, Methanol-d₄) δ 8.54 (s, 1H), 4.42 (d, 1H), 4.21 (d, 1H), 3.71-3.62 (m, 2H), 3.51 (td, 2H), 3.40 (dt, 4H), 3.16 (s, 1H), 3.04 (s, 1H), 2.81 (s, 1H), 2.67 (s, 4H), 2.38 (s, 1H), 1.94 (dq, 6H), 1.51 (s, 3H), 1.43 (dd, 3H), 1.35 (s, 3H), 1.30 (s, 3H), 1.28 (s, 5H), 0.94 (s, 2H), 0.84 (s, 1H).

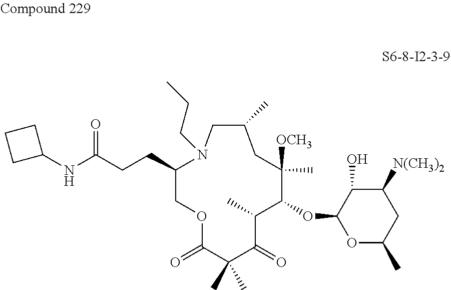

N-Cyclobutyl-3-((3R,6R,8R,9R,10R)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-11,13-dioxo-4-propyl-1-oxa-4-azacyclotridecan-3-yl)propanamide (S7-7-I2-3-9)

Prepared according to the methods of S7-7-I2-3-1, substituting cyclobutanamine to give S7-7-I2-3-9 as a formate salt. MS (ESI+) m/z: 341.2 [M+2H]2+, 681.4 [M+H]+; ¹H NMR (400 MHz, Methanol-d₄) δ 8.54 (s, 1H), 4.41 (d, 1H), 4.28 (p, 1H), 3.97 (d, 1H), 3.68-3.60 (m, 2H), 3.36 (t, 2H), 3.09 (s, 1H), 2.96 (s, 1H), 2.78 (s, 2H), 2.59 (d, 7H), 2.33-2.21 (m, 3H), 2.21-2.13 (m, 1H), 1.99-1.87 (m, 4H), 1.73 (dt, 3H), 1.52 (s, 3H), 1.34 (s, 3H), 1.30 (s, 1H), 1.28 (d, 7H), 0.93 (q, 3H), 0.84 (s, 2H).

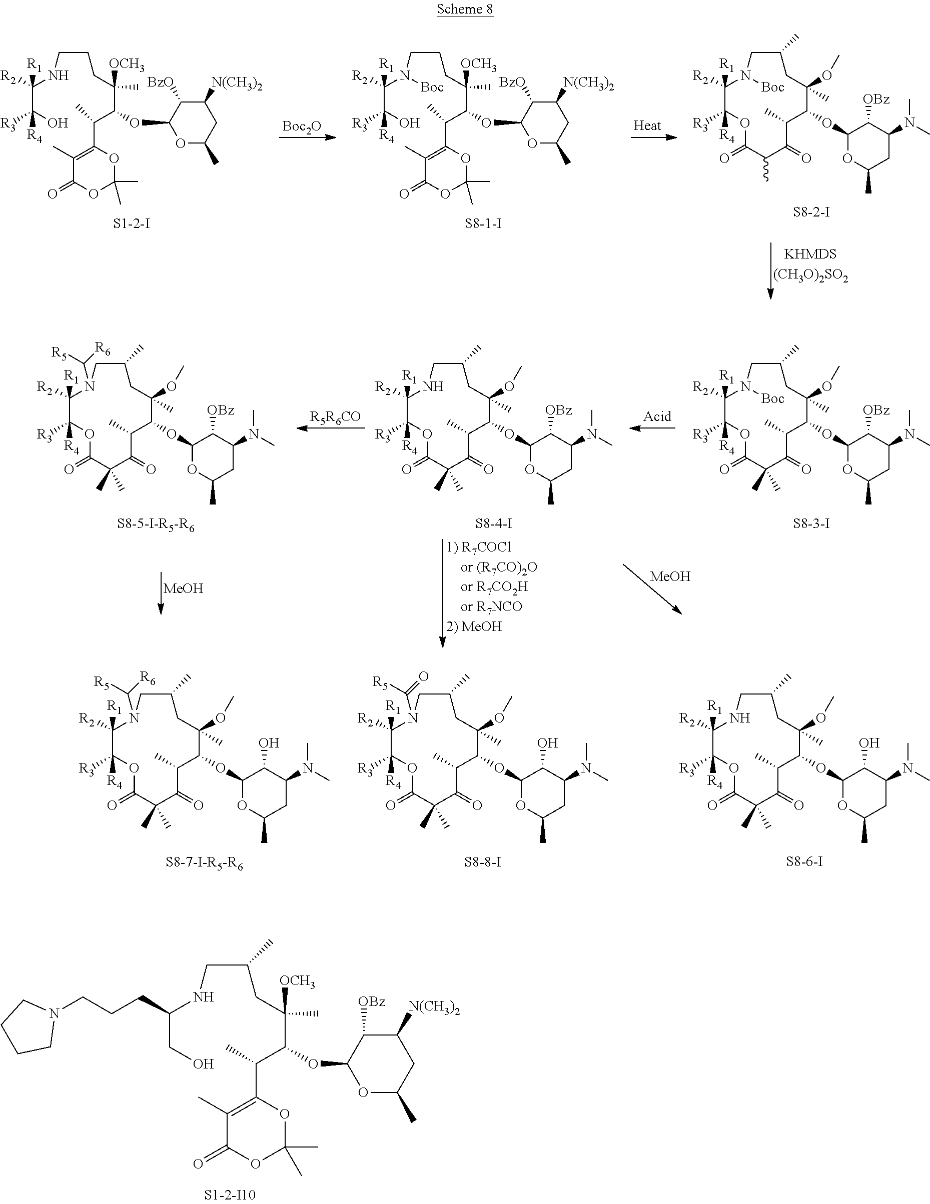

(2S,3R,4S,6R)-4-(Dimethylamino)-2-(((2R,3R,4R,6R)-7-(((R)-1-hydroxy-5-(pyrrolidin-1-yl)pentan-2-yl)amino)-4-methoxy-4,6-dimethyl-2-(2,2,5-trimethyl-4-oxo-4H-1,3-dioxin-6-yl)heptan-3-yl)oxy)-6-methyltetrahydro-2H-pyran-3-yl benzoate (S1-2-I10)

S1-1 (0.675 g, 1.14 mmol) and (R)-2-amino-5-(pyrrolidin-1-yl)pentan-1-ol (I10, 234 mg, 1.36 mmol) were dissolved in EtOH (5 mL), and Ti(OEt)4 (0.72 mL, 2.96 mmol) was added. After 30 min, a small aliquot was removed from the reaction mixture and was added to a suspension of a small amount of NaBH₄ in MeOH. LC/MS analysis showed complete conversion. NaBH₄ (107 mg, 3.42 mmol) was added. When gas evolution ceased, 30% aqueous NH₄OH (3 mL) was added, and the mixture was filtered through a pad of Celite®, washing with EtOAc. The filtrate was washed with brine, was dried over Na₂SO₄, was filtered, and was concentrated to give S1-2-I10. The material was used without further purification. MS (ESI+) m/z: 746.49 [M+H]⁺, 374.00 [M+2H]²⁺, 249.66 [M+3H]³⁺.

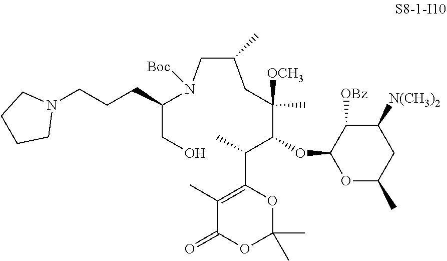

(2S,3R,4S,6R)-2-(((2R,3R,4R,6R)-7-((tert-Butoxycarbonyl)((R)-1-hydroxy-5-(pyrrolidin-1-yl)pentan-2-yl)amino)-4-methoxy-4,6-dimethyl-2-(2,2,5-trimethyl-4-oxo-4H-1,3-dioxin-6-yl)heptan-3-yl)oxy)-4-(dimethylamino)-6-methyltetrahydro-2H-pyran-3-yl benzoate (S8-1-I10)

In a 40 mL vial was a solution of S1-2-I10 (850 mg, 1.13 mmol) in dichloromethane (5 mL) to give a yellow solution which was stirred at rt. Boc2O (0.33 mL, 1.46 mmol) was added In an portion and allowed to stir at rt for 2 hours. The reaction was diluted with dichloromethane and poured into satd aq NaHCO3. The aqueous phase was extracted with dichloromethane and the combined organic phases were dried over MgSO4, filtered and concentrated. The residue was purified on 24 g silica gel (elution with 0-6% MeOH-dichloromethane) to give S8-1-I10 (520 mg, 54% in two steps). MS (ESI+) m/z: 846.5 [M+H]+, 423.8 [M+2H]2+.

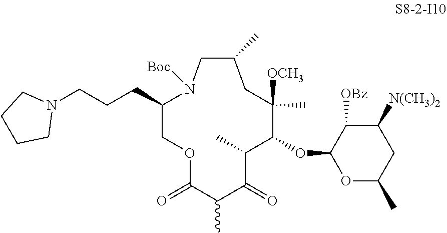

tert-Butyl (3R,6R,8R,9R,10R)-9-(((2S,3R,4S,6R)-3-(benzoyloxy)-4-(dimethylamino)-6-methyltetrahydro-2H-pyran-2-yl)oxy)-8-methoxy-6,8,10,12-tetramethyl-11,13-dioxo-3-(3-(pyrrolidin-1-yl)propyl)-1-oxa-4-azacyclotridecane-4-carboxylate (S8-2-I10): The flask was fitted with a reflux condenser and the condenser was flame dried under vacuum, allowed to cool and backfilled with nitrogen. The solution of S8-2-I10 (520 mg, 0.614 mmol) in chlorobenzene (150 mL) was added via cannula and the flask was placed under mild vacuum and sonicated for 2 minutes, then backfilled with nitrogen. The degassing procedure was repeated, then the mixture was heated at a bath temperature of 155° C. for 16 hours. The reaction was allowed to cool to rt and was concentrated. The residue was purified on 24 g of silica gel (elution with 0-10% MeOH-dichloromethane+0.5% of 30% aq NH4OH) to give S8-2-I10 (395 mg, 82%). MS (ESI+) m/z: 394.8 [M+2H]²⁺, 788.5 [M+H]⁺.

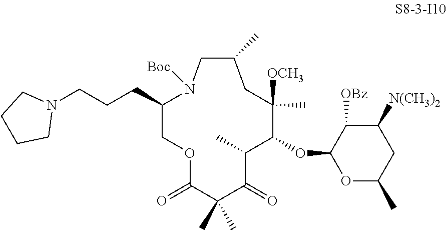

tert-Butyl (3R,6R,8R,9R,10R)-9-(((2S,3R,4S,6R)-3-(benzoyloxy)-4-(dimethylamino)-6-methyltetrahydro-2H-pyran-2-yl)oxy)-8-methoxy-6,8,10,12,12-pentamethyl-11,13-dioxo-3-(3-(pyrrolidin-1-yl)propyl)-1-oxa-4-azacyclotridecane-4-carboxylate (S8-3-I10)

In a 20 mL vial was a solution of S8-2-I10 (360 mg, 0.46 mmol) in 1,2-dimethoxyethane (5 mL) precooled at −60° C. KHMDS (0.68 mL, 0.68 mmol) was added dropwise. The reaction mixture was stirred at −60° C. for 20 min. Then Me₂SO₄ (65 μL, 0.68 mmol) was added. The reaction mixture was allowed to warm to −15° C. LC/MS shows full conversion. The reaction was quenched by adding trimethylamine (0.88 mL) and the resulting mixture was diluted with dichloromethane and saturated NaHCO3 was added. The aqueous layer was extracted with dichloromethane and the combined organic layers were dried over MgSO4, filtered and concentrated. The residue was purified on 4 g of silica gel (elution with 0-10% MeOH-dichloromethane+0.5% of 30% aq NH4OH) to give S8-3-I10 (145 mg, 40%). MS (ESI+) m/z: 401.8 [M+2H]2+, 802.5 [M+H]+.

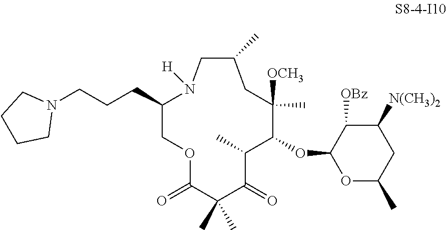

(2S,3R,4S,6R)-4-(Dimethylamino)-2-(((3R,6R,8R,9R,10R)-8-methoxy-6,8,10,12,12-pentamethyl-11,13-dioxo-3-(3-(pyrrolidin-1-yl)propyl)-1-oxa-4-azacyclotridecan-9-yl)oxy)-6-methyltetrahydro-2H-pyran-3-yl benzoate (S8-4-I10)

In a 20 mL flask was a solution of S8-3-I10 (135 mg, 0.17 mmol) in 1.5 mL of DCM at rt. TFA (0.52 mL, 6.74 mmol) was added and the mixture was stirred at rt. The reaction was complete by UPLC. The mixture was diluted with 30 mL of DCM and 30 mL of satd aq NaHCO3 was added. The aqueous phase was extracted 3× w/DCM and the combined organic phases were dried over MgSO4, filtered and concentrated to give S8-4-I10. The product was used as is without further purification. MS (ESI+) m/z: 234.8 [M+3H]3+, 351.8 [M+2H]2+, 702.5 [M+H]+.

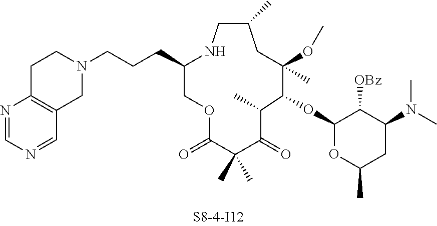

(2S,3R,4S,6R)-2-(((3R,6R,8R,9R,10R)-3-(3-(7,8-dihydropyrido[4,3-d]pyrimidin-6(5H)-yl)propyl)-8-methoxy-6,8,10,12,12-pentamethyl-11,13-dioxo-1-oxa-4-azacyclotridecan-9-yl)oxy)-4-(dimethylamino)-6-methyltetrahydro-2H-pyran-3-yl benzoate (S8-4-I12) Prepared according to the methods of S8-4-I10, substituting intermediate 112 to give S8-4-I12. MS (ESI+) m/z: 383.58 [M+2H]2+, 766.00 [M+H]+.

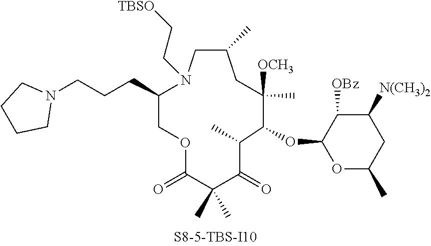

(2S,3R,4S,6R)-2-(((3R,6R,8R,9R,10R)-4-(2-((tert-Butyldimethylsilyl)oxy)ethyl)-8-methoxy-6,8,10,12,12-pentamethyl-11,13-dioxo-3-(3-(pyrrolidin-1-yl)propyl)-1-oxa-4-azacyclotridecan-9-yl)oxy)-4-(dimethylamino)-6-methyltetrahydro-2H-pyran-3-yl benzoate (S8-5-TBS-I10)

Compound S8-4-I10 (105 mg, 0.15 mmol) was dissolved in dry methylene chloride (1 mL) and 2-((tert-butyldimethylsilyl)oxy)acetaldehyde (0.042 mL, 0.223 mmol) and AcOH (0.026 mL, 0.45 mmol) was added. Then NaBH(OAc)₃ (63 mg, 0.30 mmol) was added to the reaction mixture In an portion. The reaction was allowed to stir at rt for 2 h and LC/MS shows full conversion. The reaction was quenched by adding saturated NaHCO₃(5 mL) and the aqueous layer was extracted with methylene chloride three times (10 mL). The combined organic layers were dried over MgSO4, filtered and concentrated. The residue was purified on 4 g of silica gel (elution with 0-10% MeOH-dichloromethane+0.5% of 30% aq NH4OH) to give 61 mg of S8-5-TBS-I10 (48% yield). MS (ESI+) m/z: 287.5 [M+3H]3+, 430.8 [M+2H]2+, 860.6 [M+H]+.

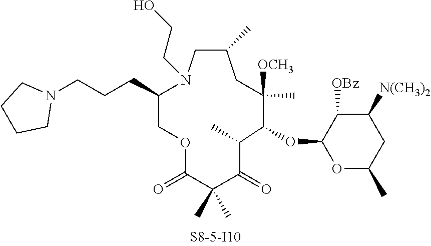

(2S,3R,4S,6R)-4-(Dimethylamino)-2-(((3R,6R,8R,9R,10R)-4-(2-hydroxyethyl)-8-methoxy-6,8,10,12,12-pentamethyl-11,13-dioxo-3-(3-(pyrrolidin-1-yl)propyl)-1-oxa-4-azacyclotridecan-9-yl)oxy)-6-methyltetrahydro-2H-pyran-3-yl benzoate (S8-5-I10)

S8-5-TBS-I10 (61 mg, 0.071 mmol) was dissolved in dry THF (2 mL) and TBAF (1M in THF, 0.21 mL, 0.021 mmol) was added at room temperature. The reaction mixture was stirred at rt for 2 h and was concentrated. The residue was purified on 4 g of silica gel (elution with 0-20% MeOH— dichloromethane+0.5% of 30% aq NH4OH) to give S8-5-I10 (46 mg, 87%). MS (ESI+) m/z: 249.5 [M+3H]3+, 373.8 [M+2H]2+, 746.5 [M+H]+.

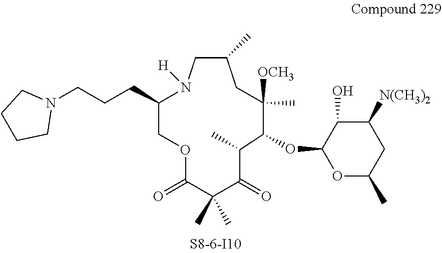

(3R,6R,8R,9R,10R)-9-(((2S,3R,4S,6R)-4-(Dimethylamino)-3-hydroxy-6-methyltetrahydro-2H1-pyran-2-yl)oxy)-8-methoxy-6,8,10,12,12-pentamethyl-3-(3-(pyrrolidin-1-yl)propyl)-1-oxa-4-azacyclotridecane-11,13-dione (S8-6-I10)

S8-4-I10 (25 mg, 0.036 mmol) was dissolved in MeOH (1 mL), and the reaction mixture was heated to 40° C. (external temp.) overnight. The reaction mixture was cooled to rt and was concentrated under reduced pressure. The material was purified by HIPLC (Atlantis T3 column, 5-30% MeCN-water-0.1% HCO2H) to give 6.52 mg of S58-6-I10 as a formate salt. MS (ESI+) m/z: 200.1 [M+3H]3+, 299.7 [M+2H]2+, 598.4 [M+H]+; 1H NMR (400 MHz, Methanol-d) δ8.56 (s, 1H), 4.39 (d, 1H), 4.13 (d, 1H), 3.87 (dd, 1H), 3.63 (ddt, 1H), 3.49 (dt, 1H), 3.35-3.27 (m, 3H), 3.11 (d, 1H), 3.10-2.96 (m, 3H), 2.94 (d, 5H), 2.86 (s, 1H), 2.69 (dd, 1H), 2.50 (s, 4H), 2.44 (t, 3H), 2.04-1.96 (m, 4H), 1.91-1.78 (m, 4H), 1.71-1.57 (m, 3H), 1.48 (s, 3H), 1.44 (d, 1H), 1.41-1.32 (m, 8H), 1.27 (d, 5H), 1.02 (d, 3H).

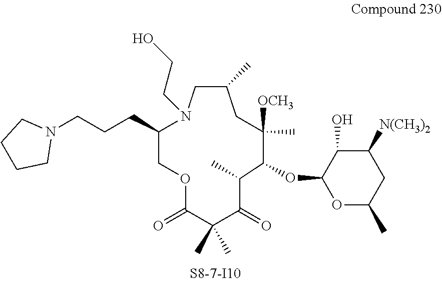

(3R,6R,8R,9R,10R)-9-(((2S,3R,4S,6R)-4-(Dimethylamino)-3-hydroxy-6-methyltetrahydro-2H-pyran-2-yl)oxy)-4-(2-hydroxyethyl)-8-methoxy-6,8,10,12,12-pentamethyl-3-(3-(pyrrolidin-1-yl)propyl)-1-oxa-4-azacyclotridecane-11,13-dione (S7-7-I10)

Prepared according to the method of S8-6-I10 and starting from S8-5-I10 to provide the title compound as a formate salt. MS (ESI+) m/z: 214.8 [M+3H]3+, 321.8 [M+2H]2+, 642.5 [M+H]+; 1H NMR (400 MHz, Methanol-d) δ8.49 (s, 2H), 4.52 (s, 1H), 4.44 (d, 1H), 4.04 (d, 1H), 3.84 (s, 1H), 3.70 (ddd, 1H), 3.55 (d, 3H), 3.49-3.33 (m, 2H), 3.33 (s, 3H), 3.30 (p, 1H), 3.22-3.08 (m, 2H), 3.03 (s, 1H), 2.81 (s, 6H), 2.55 (s, 2H), 2.26 (s, 1H), 2.08 (d, 1H), 2.09-2.00 (m, 3H), 2.03-1.91 (m, 1H), 1.83-1.74 (m, 2H), 1.58-1.45 (m, 1H), 1.45 (s, 2H), 1.39 (s, 1H), 1.35 (s, 2H), 1.33-1.24 (m, 8H), 1.15 (s, 1H), 0.84 (d, 3H).

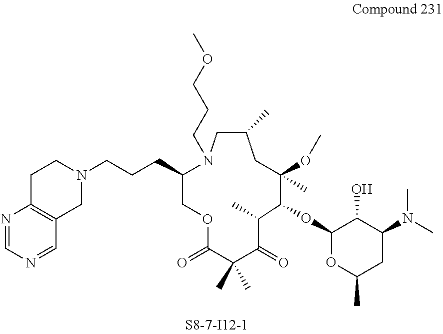

(3R,6R,8R,9R,10R)-3-(3-(7,8-dihydropyrido[4,3-d]pyrimidin-6(5H)-yl)propyl)-9-(((2S,3R,4S,6R)-4-(dimethylamino)-3-hydroxy-6-methyltetrahydro-2H-pyran-2-yl)oxy)-8-methoxy-4-(3-methoxypropyl)-6,8,10,12,12-pentamethyl-1-oxa-4-azacyclotridecane-11,13-dione (S8-7-I12-1)

Prepared according to the methods of S8-7-I10, starting from S8-4-I12, and substituting 3-methoxypropanal to provide 20.03 mg of S8-7-I12-1 as a formate salt. MS (ESI+) m/z: 367.57 [M+2H]2+, 734.03 [M+H]+; ¹H NMR (400 MHz, Methanol-d₄) δ 8.93 (s, 1H), 8.54 (s, 1H), 8.50 (s, 2H), 4.47 (dd, 1H), 4.32-4.06 (m, 2H), 3.80-3.65 (m, 4H), 3.58-3.36 (m, 6H), 3.04 (t, 3H), 3.02-2.88 (m, 6H), 2.84 (s, 7H), 2.77-2.62 (m, 3H), 2.11-2.00 (m, 2H), 2.00-1.63 (m, 7H), 1.60-1.54 (m, 1H), 1.52 (s, 4H), 1.38 (d, 5H), 1.36-1.30 (m, 8H), 1.10-0.80 (m, 3H).

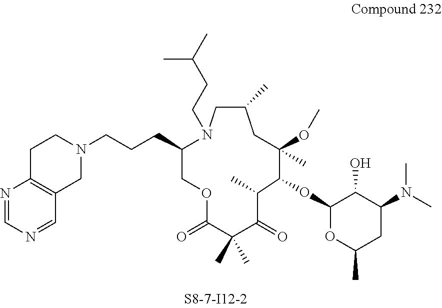

(3R,6R,8R,9R,10R)-3-(3-(7,8-dihydropyrido[4,3-d]pyrimidin-6(5H)-yl)propyl)-9-(((2S,3R,4S,6R)-4-(dimethylamino)-3-hydroxy-6-methyltetrahydro-2H-pyran-2-yl)oxy)-4-isopentyl-8-methoxy-6,8,10,12,12-pentamethyl-1-oxa-4-azacyclotridecane-11,13-dione (S8-7-I12-2)

Prepared according to the methods of S8-7-I10, starting from S8-4-I12, and substituting 3-methylbutanal to provide 16.47 mg of S8-7-I12-2 as a formate salt. MS (ESI+) m/z: 366.51 [M+2H]2+, 731.94 [M+H]+; ¹H NMR (400 MHz, Methanol-d₄) δ 8.94 (s, 1H), 8.55 (s, 1H), 8.51 (s, 2H), 4.47 (d, 1H), 4.22 (s, 1H), 3.74 (q, 4H), 3.51 (dd, 1H), 3.41 (ddd, 2H), 3.04 (t, 3H), 3.01-2.89 (m, 6H), 2.84 (s, 7H), 2.78-2.61 (m, 3H), 2.05 (d, 3H), 1.87-1.62 (m, 5H), 1.60-1.54 (m, 2H), 1.52 (s, 6H), 1.38 (d, 5H), 1.34 (t, 8H), 0.94 (t, 9H).

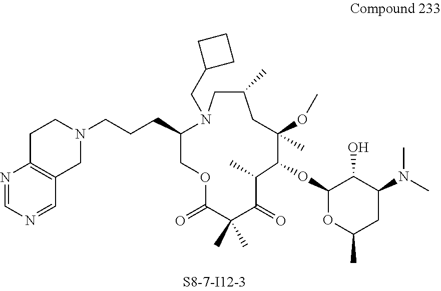

(3R,6R,8R,9R,10R)-4-(cyclobutylmethyl)-3-(3-(7,8-dihydropyrido[4,3-d]pyrimidin-6(5H)-yl)propyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-1-oxa-4-azacyclotridecane-11,13-dione (S8-7-112-3)

Prepared according to the methods of S8-7-I10, starting from S8-4-112, and substituting cyclobutanecarboxaldehyde to provide 16.89 mg of the S8-7-112-3 as a formate salt. MS (ESI+) m/z: 365.56 [M+2H]2+, 730.09 [M+H]+; ¹H NMR (400 MHz, Methanol-d₄) δ 8.94 (s, 1H), 8.55 (s, 1H), 8.51 (s, 2H), 4.47 (d, 1H), 3.81-3.67 (m, 4H), 3.51 (dd, 1H), 3.41 (ddd, 2H), 3.11-2.87 (m, 9H), 2.84 (s, 7H), 2.77-2.55 (m, 4H), 2.16 (s, 1H), 2.11-2.00 (m, 3H), 1.96-1.80 (m, 6H), 1.78-1.63 (m, 3H), 1.60-1.54 (m, 1H), 1.52 (s, 4H), 1.37-1.29 (m, 13H), 0.89 (s, 3H).

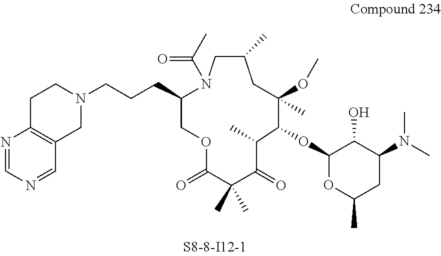

(3R,6R,8R,9R,10R)-4-acetyl-3-(3-(7,8-dihydropyrido[4,3-d]pyrimidin-6(5H)-yl)propyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-1-oxa-4-azacyclotridecane-11,13-dione (S8-8-I12-1): To a solution of S8-4-I12 (42 mg, 0.055 mmol) in dichloromethane (1 mL) was added the acetic anhydride (10.2 μL, 0.1 mmol). The reaction mixture was stirred at room temperature for 1 hr. It was quenched with NaHCO₃ and extracted with dichloromethane. The organic extracts were combined, dried over sodium sulfate, and filtered through a small pad of silica (washed with 10% MeOH/DCM). The filtered solution was concentrated. The crude residue was dissolved in methanol, and heated to 45C overnight. UPLC showed complete conversion to the desired product. Solvent was removed and the crude residue was purified with prep-HPLC, eluting with 5-20% MeCN—H2O-0.5% formic acid to provide 8.02 mg of S8-8-I12-1 as a formate salt. MS (ESI+) m/z: 352.46 [M+2H]2+, 703.91 [M+H]+; ¹H NMR (400 MHz, Methanol-d₄) δ 8.91 (s, 1H), 8.53 (s, 1H), 8.47 (s, 1H), 4.44 (d, 1H), 4.41-4.33 (m, 1H), 4.09 (dd, 1H), 3.98 (dd, 2H), 3.87 (dd, 1H), 3.76-3.67 (m, 3H), 3.64-3.54 (m, 1H), 3.50-3.36 (m, 2H), 3.03 (t, 2H), 2.95-2.88 (m, 2H), 2.76 (dd, 1H), 2.72-2.61 (m, 5H), 2.38 (s, 2H), 2.07-1.99 (m, 1H), 1.94-1.84 (m, 1H), 1.80 (d, 2H), 1.71-1.56 (m, 6H), 1.56-1.44 (m, 1H), 1.32 (dd, 4H), 1.29 (s, 5H), 1.24 (d, 3H), 1.10 (dd, 1H), 0.94 (d, 3H).

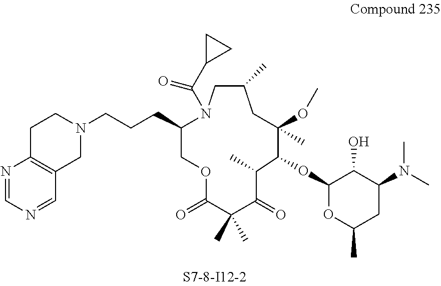

(3R,6R,8R,9R,10R)-4-(cyclopropanecarbonyl)-3-(3-(7,8-dihydropyrido[4,3-d]pyrimidin-6(5H)-yl)propyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-1-oxa-4-azacyclotridecane-11,13-dione (S8-8-I12-2)

Prepared according to the methods of S8-8-I12-1 substituting cyclopropanecarbonyl chloride to provide 5.02 mg of S8-8-I12-2 as a formate salt. MS (ESI+) m/z: 365.56 [M+2H]2+, 730.14 [M+H]+; ¹H NMR (400 MHz, Methanol-d₄) δ 8.91 (s, 1H), 8.55 (s, 1H), 8.52 (s, 1H), 4.41 (d, 1H), 4.13-4.05 (m, 1H), 4.05-3.96 (m, 2H), 3.91 (t, 1H), 3.70 (s, 2H), 3.68-3.61 (m, 2H), 3.42 (dd, 1H), 3.02 (t, 2H), 2.97-2.79 (m, 3H), 2.76 (s, 5H), 2.70-2.61 (m, 7H), 2.39-2.30 (m, 1H), 2.01-1.82 (m, 3H), 1.80 (d, 1H), 1.77-1.68 (m, 1H), 1.65 (s, 5H), 1.57-1.43 (m, 2H), 1.35-1.26 (m, 10H), 1.24 (d, 4H), 1.21-1.10 (m, 1H), 1.09-0.99 (m, 2H), 0.94 (d, 4H), 0.88-0.78 (m, 2H).

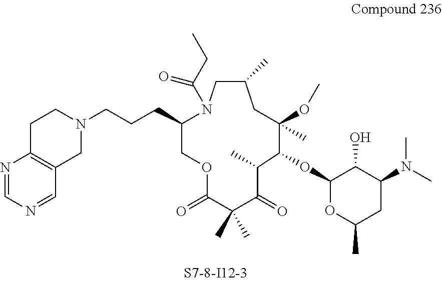

(3R,6R,8R,9R,10R)-3-(3-(7,8-dihydropyrido[4,3-d]pyrimidin-6(5H)-yl)propyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-4-propionyl-1-oxa-4-azacyclotridecane-11,13-dione (S7-8-I12-3)

Prepared according to the methods of S8-8-112-1 substituting acetyl chloride to provide 5.38 mg of S8-8-112-3 as a formate salt. MS (ESI+) m/z: 359.56 [M+2H]2+, 718.05 [M+H]+; ¹H NMR (400 MHz, Methanol-d₄) δ 8.80 (s, 1H), 8.41 (s, 2H), 4.39-4.34 (m, 1H), 4.32 (d, 1H), 3.97 (dd, 1H), 3.87 (t, 2H), 3.77 (t, 1H), 3.60 (s, 3H), 3.50-3.39 (m, 1H), 3.37-3.24 (m, 2H), 2.91 (t, 2H), 2.84-2.75 (m, 2H), 2.71 (s, 6H), 2.67 (s, 1H), 2.67-2.63 (m, 1H), 2.60-2.50 (m, 6H), 1.90 (d, 1H), 1.81-1.74 (m, 1H), 1.67 (d, 2H), 1.60-1.49 (m, 3H), 1.48 (s, 3H), 1.46-1.36 (m, 2H), 1.21 (d, 4H), 1.18 (s, 6H), 1.09 (q, 6H), 0.98 (dd, 1H), 0.82 (d, 3H).

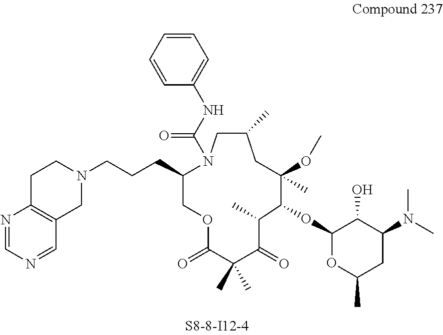

(3R,6R,8R,9R,10R)-3-(3-(7,8-dihydropyrido[4,3-d]pyrimidin-6(5H)-yl)propyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-11,13-dioxo-N-phenyl-1-oxa-4-azacyclotridecane-4-carboxamide (S8-8-I12-4)

Prepared according to the methods of S8-8-I12-1 substituting phenyl isocyanate to provide 9.05 mg of the title compound as a formate salt. MS (ESI+) m/z: 391.09 [M+2H]2+, 780.89 [M+H]+; ¹H NMR (400 MHz, Methanol-d₄) δ 8.89 (s, 1H), 8.55 (s, 1H), 8.48 (s, 1H), 7.44-7.33 (m, 2H), 7.33-7.23 (m, 2H), 7.09-7.00 (m, 1H), 4.57 (s, 1H), 4.40 (d, 1H), 4.14 (dd, 1H), 3.98 (d, 2H), 3.87 (s, 1H), 3.76-3.60 (m, 4H), 3.43-3.37 (m, 1H), 3.25 (d, 1H), 3.01 (t, 2H), 2.96-2.84 (m, 3H), 2.74 (s, 6H), 2.70-2.63 (m, 5H), 2.05-1.94 (m, 2H), 1.91-1.82 (m, 2H), 1.80-1.68 (m, 2H), 1.65 (s, 4H), 1.48 (q, 1H), 1.31 (s, 6H), 1.29 (s, 3H), 1.17 (d, 3H), 1.12 (d, 1H), 0.95 (d, 3H).

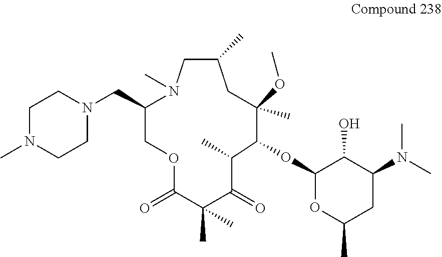

(3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-((4-methylpiperazin-1-yl)methyl)-1-oxa-4-azacyclotridecane-11,13-dione

Prepared according to the methods of S3-2-I4-1-2 from S3-2-I7-1 to provide the title compound as a formate salt. MS (ESI+) m/z: 613.01 [M+H]+; ¹H NMR (400 MHz, Methanol-d4) δ 8.55 (S, 1H), 4.55-4.36 (m, 2H), 4.31-4.09 (m, 2H), 3.66 (m, 2H), 3.58-3.44 (m, 1H), 2.97 (br s, 6H), 2.80-2.41 (m, 19H), 2.33 (s, 4H), 1.90 (br d, J=11.5 Hz, 2H), 1.84-1.68 (m, 1H), 1.55 (br s, 3H), 1.44-1.21 (m, 14H), 0.99 (br s, 3H).

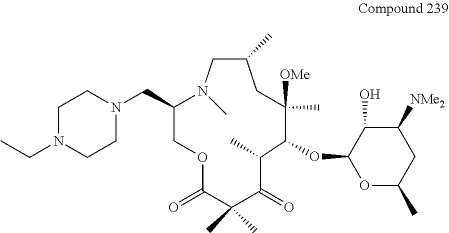

(3R,6R,8R,9R,10R)-9-(((2S,3R,4S,6R)-4-(dimethylamino)-3-hydroxy-6-methyltetrahydro-2H-pyran-2-yl)oxy)-3-((4-ethylpiperazin-1-yl)methyl)-8-methoxy-4,6,8,10,12,12-hexamethyl-1-oxa-4-azacyclotridecane-11,13-dione

Prepared according to the methods of S3-2-I4-1-2 from S3-2-I7-1 to provide the title compound as a formate salt. MS (ESI+) m/z: 627.4 [M+H]+; ¹H NMR (400 MHz, Methanol-d4) δ 8.55 (s, 0.4H), 4.52-4.24 (m, 2H), 4.20-3.95 (m, 2H), 3.71-3.52 (m, 2H), 3.29-3.16 (m, 2H), 3.01-2.83 (s, 3H), 2.82-2.19 (m, 24H), 2.17-1.87 (m, 2H), 1.86-1.69 (m, 2H), 1.60-1.44 (3, 3H), 1.41-1.20 (m, 14H), 1.12 (t, J=7.2 Hz, 4H), 1.00-0.81 (m, 3H).

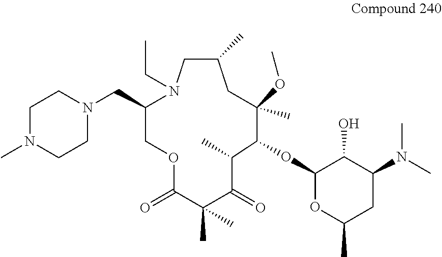

(3R,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-3-((4-methylpiperazin-1-yl)methyl)-1-oxa-4-azacyclotridecane-11,13-dione

Prepared according to the methods of S3-2-I4-1-2 from S3-2-17-2 and acetaldehyde to provide the title compound as a formate salt. MS (ESI+) m/z: 627.12 [M+H]+; ¹H NMR (400 MHz, CDCl₃) δ 8.49 (s, 2H), 4.69 (s, 1H), 4.46 (d, 1H), 4.29 (s, 1H), 4.15 (d, 1H), 3.73 (m, 1H), 3.54 (s, 1H), 3.51-3.33 (m, 2H), 3.04 (s, 3H), 2.95 (s, 6H), 2.81 (s, 8H), 2.61 (s, 7H), 2.09-1.96 (m, 2H), 1.53 (d, 4H), 1.42-1.28 (m, 12H), 1.25 (t, 3H), 0.99 (d, 3H).

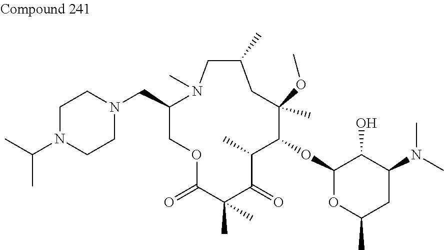

(3R,6R,8R,9R,10R)-9-(((2S,3R,4S,6R)-4-(dimethylamino)-3-hydroxy-6-methyltetrahydro-2H-pyran-2-yl)oxy)-3-((4-isopropylpiperazin-1-yl)methyl)-8-methoxy-4,6,8,10,12,12-hexamethyl-1-oxa-4-azacyclotridecane-11,13-dione

Prepared according to the methods of S3-2-I4-1-2 from S3-2-I7-1 and acetone to provide the title compound as a free base. MS (ESI+) m/z: 641.09 [M+H]+; ¹H NMR (400 MHz, CDCl₃) δ 8.54 (s, 2H), 4.59 (d, J=1H), 4.46 (d, 1H), 4.34 (t, 1H), 4.24 (d, 1H), 3.86-3.66 (m, 2H), 3.46 (m, 2H), 3.12 (m, 2H), 3.00 (s, 7H), 2.77 (d, 14H), 2.61 (dd, 2H), 2.15 (s, 1H), 2.06-1.95 (m, 1H), 1.70 (s, 2H), 1.54 (s, 3H), 1.53-1.44 (m, 1H), 1.39 (s, 6H), 1.33 (dd, 6H), 1.25 (d, 6H), 1.03 (d, 3H).

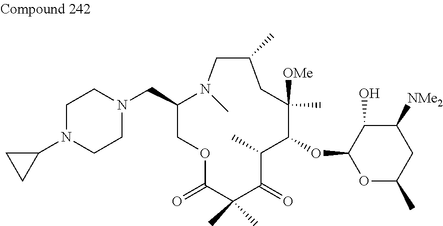

(3R,6R,8R,9R,10R)-3-((4-cyclopropylpiperazin-1-yl)methyl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione

Prepared according to the methods of S3-2-I4-1-2 from S3-2-I7-1 and cyclopropanone to provide the title compound as a formate salt. MS (ESI+) m/z: 719.3 [M+H]+; ¹H NMR (400 MHz, Methanol-d4) δ 8.55 (s, 0.8H), 4.67-4.36 (m, 2H), 4.34-4.05 (m, 2H), 3.84-3.60 (m, 2H), 3.59-3.45 (m, 1H), 3.42-3.33 (m, 1H), 3.21-2.87 (m, 5H), 2.84-2.30 (m, 20H), 2.26-1.95 (m, 1H), 1.94-1.62 (m, 3H), 1.61-1.48 (m, 3H), 1.46-1.19 (m, 13H), 1.18-0.81 (m, 4H), 0.56-0.34 (m, 3H).

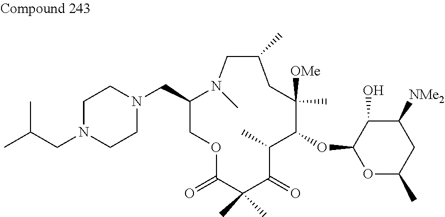

(3R,6R,8R,9R,10R)-9-(((2S,3R,4S,6R)-4-(dimethylamino)-3-hydroxy-6-methyltetrahydro-2H-pyran-2-yl)oxy)-3-((4-isobutylpiperazin-1-yl)methyl)-8-methoxy-4,6,8,10,12,12-hexamethyl-1-oxa-4-azacyclotridecane-11,13-dione

Prepared according to the methods of S3-2-I4-1-2 from S3-2-I7-1 and t-butylcarboxaldehyde to provide the title compound as a formate salt. MS (ESI+) m/z: 655.4 [M+H]+; ¹H NMR (400 MHz, Methanol-d4) δ 8.55 (s, 0.4H), 4.48-4.31 (m, 2H), 4.24-4.01 (m, 2H), 3.61 (m, 2H), 3.29 (m, 1H), 2.92 (br s, 3H), 2.82-2.71 (m, 2H), 2.68-2.27 (m, 20H), 2.14 (d, J=7.3 Hz, 3H), 1.89-1.74 (m, 3H), 1.52 (br s, 3H), 1.43-1.18 (m, 15H), 0.99-0.83 (m, 9H).

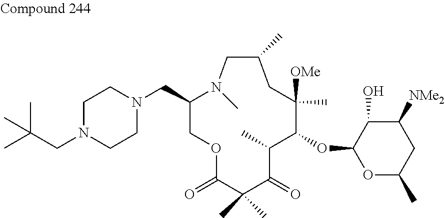

(3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-((4-neopentylpiperazin-1-yl)methyl)-1-oxa-4-azacyclotridecane-11,13-dione

Prepared according to the methods of S3-2-I4-1-2 from S3-2-I7-1 and isobutyraldehyde to provide the title compound as a formate salt. MS (ESI+) m/z: 669.3 [M+H]+; ¹H NMR (400 MHz, Methanol-d4) δ 8.55 (s, 0.2H), 4.90 (m, 5H), 4.66-4.51 (m, 1H), 4.38 (m, 2H), 4.22-3.86 (m, 2H), 3.79-3.44 (m, 3H), 3.35 (m, 2H), 3.27 (m, 1H), 2.99-2.82 (m, 3H), 2.76-2.26 (m, 19H), 2.19-1.97 (m, 4H), 1.85-1.67 (m, 2H), 1.57-1.44 (m, 3H), 1.41-1.20 (m, 12H), 1.00-0.81 (m, 10H).

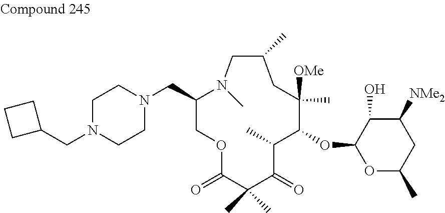

(3R,6R,8R,9R,10R)-3-((4-(cyclobutylmethyl)piperazin-1-yl)methyl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione

Prepared according to the methods of S3-2-I4-1-2 from S3-2-I7-1 and cyclobutanecarboxaldehyde to provide the title compound as a formate salt. MS (ESI+) m/z: 667.3 [M+H]+; ¹H NMR (400 MHz, Methanol-d4) δ 4.44-4.24 (m, 2H), 4.14-3.91 (m, 2H), 3.76-3.52 (m, 2H), 3.29-3.10 (m, 2H), 2.96-2.80 (m, 3H), 2.71-2.23 (m, 24H), 2.13-1.66 (m, 10H), 1.48 (s, 3H), 1.38-1.18 (m, 13H), 1.15-1.04 (m, 1H), 1.00-0.80 (m, 3H).

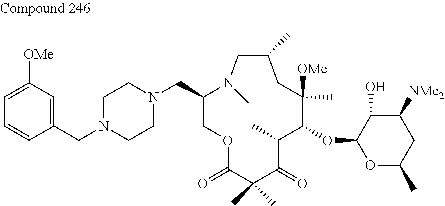

(3R,6R,8R,9R,10R)-9-(((2S,3R,4S,6R)-4-(dimethylamino)-3-hydroxy-6-methyltetrahydro-2H-pyran-2-yl)oxy)-8-methoxy-3-((4-(3-methoxybenzyl)piperazin-1-yl)methyl)-4,6,8,10,12,12-hexamethyl-1-oxa-4-azacyclotridecane-11,13-dione

Prepared according to the methods of S3-2-I4-1-2 from S3-2-I7-1 and 3-methoxybenzaldehyde to provide the title compound as a formate salt. MS (ESI+) m/z: 719.3 [M+H]+; ¹H NMR (400 MHz, Methanol-d4) δ 8.55 (br s, 0.3H), 7.23 (t, 1H), 6.93-6.87 (m, 2H), 6.83 (dd, 1H), 4.38 (br d, 2H), 4.22-3.89 (m, 2H), 3.79 (s, 3H), 3.59 (br dd, 2H), 3.50 (s, 2H), 3.29-3.11 (m, 2H), 2.88 (br s, 3H), 2.76-2.18 (m, 22H), 2.01 (br s, 1H), 1.85-1.63 (m, 2H), 1.50 (br s, 3H), 1.40-1.17 (m, 14H), 1.09 (br s, 1H), 0.94-0.83 (m, 2H).

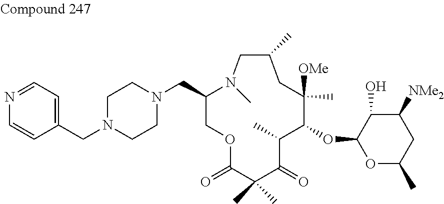

(3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-((4-(pyridin-4-ylmethyl)piperazin-1-yl)methyl)-1-oxa-4-azacyclotridecane-11,13-dione

Prepared according to the methods of S3-2-I4-1-2 from S3-2-I7-1 and 4-pyridinecarboxaldehyde to provide the title compound as a formate salt. MS (ESI+) m/z: 690.3 [M+H]+; ¹H NMR (400 MHz, Methanol-d4) δ 8.58-8.44 (m, 2H), 7.43 (d, J=5.7 Hz, 2H), 4.39 (br d, J=7.3 Hz, 2H), 4.09 (m, 2H), 3.80-3.39 (m, 5H), 3.29-3.23 (m, 1H), 3.07-2.10 (m, 25H), 1.97-1.62 (m, 3H), 1.62-1.44 (m, 3H), 1.43-1.11 (m, 14H), 1.03-0.76 (m, 3H).

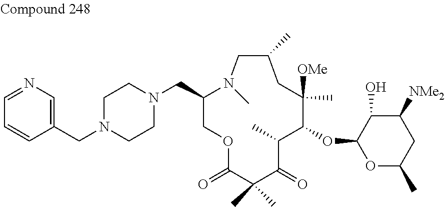

(3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-((4-(pyridin-3-ylmethyl)piperazin-1-yl)methyl)-1-oxa-4-azacyclotridecane-11,13-dione

Prepared according to the methods of S3-2-I4-1-2 from S3-2-I7-1 and 3-pyridinecarboxaldehyde to provide the title compound as a formate salt. MS (ESI+) m/z: 693.3 [M+H]+; ¹H NMR (400 MHz, Methanol-d4) δ 8.55 (s, 1H), 8.50 (d, J=1.3 Hz, 1H), 8.45 (dd, J=1.3, 4.9 Hz, 1H), 7.84 (br d, J=7.8 Hz, 1H), 7.42 (dd, J=5.0, 7.8 Hz, 1H), 4.51-4.41 (m, 2H), 4.31-4.15 (m, 2H), 3.70-3.64 (m, 1H), 3.59 (s, 2H), 3.51 (br s, 1H), 3.39-3.32 (m, 2H), 3.08-2.92 (m, 5H), 2.88-2.32 (m, 22H), 2.03 (br s, 1H), 1.90 (br d, J=12.2 Hz, 1H), 1.76 (br s, 1H), 1.55 (s, 3H), 1.45-1.40 (m, 6H), 1.40-1.34 (m, 6H), 1.05-0.92 (m, 3H).

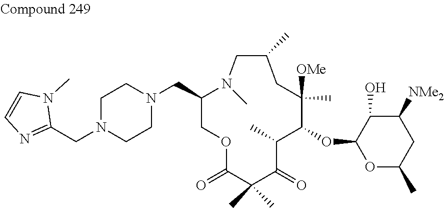

(3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-((4-((1-methyl-1H-imidazol-2-yl)methyl)piperazin-1-yl)methyl)-1-oxa-4-azacyclotridecane-11,13-dione

Prepared according to the methods of S3-2-I4-1-2 from S3-2-I7-1 and 1-methyl-1H-imidazole-2-carbaldehyde to provide the title compound as a formate salt. MS (ESI+) m/z: 690.3 [M+H]+; ¹H NMR (400 MHz, Methanol-d4) δ 8.55 (s, 0.3H), 7.06-7.01 (m, 1H), 6.85 (d, 1H), 4.49-4.22 (m, 2H), 4.20-3.91 (m, 2H), 3.78-3.67 (m, 3H), 3.64-3.54 (m, 4H), 3.42-3.33 (m, 1H), 3.28-3.22 (m, 1H), 3.22-3.06 (m, 1H), 3.03-2.80 (m, 4H), 2.77-2.18 (m, 22H), 2.02 (br s, 2H), 1.90-1.66 (m, 2H), 1.53-1.45 (m, 2H), 1.42-1.16 (m, 14H), 1.16-0.72 (m, 4H).

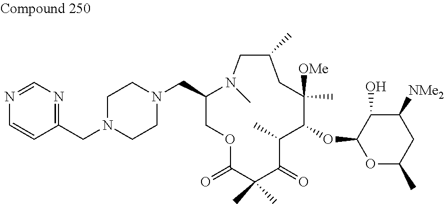

(3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-((4-(pyrimidin-4-ylmethyl)piperazin-1-yl)methyl)-1-oxa-4-azacyclotridecane-11,13-dione

Prepared according to the methods of S3-2-I4-1-2 from S3-2-I7-1 and pyrimidine-4-carbaldehyde to provide the title compound as a formate salt. MS (ESI+) m/z: 691.3 [M+H]+; ¹H NMR (400 MHz, Methanol-d4) δ 9.08 (s, 1H), 8.74 (d, 1H), 8.55 (s, 0.1H), 7.65 (d, J=5.0 Hz, 1H), 4.46-4.24 (m, 3H), 4.18-3.93 (m, 3H), 3.75-3.46 (m, 5H), 3.29-3.10 (m, 3H), 3.00-2.79 (m, 4H), 2.71-2.24 (m, 26H), 2.03 (br d, J=12.0 Hz, 2H), 1.76 (m, 3H), 1.58-1.43 (m, 4H), 1.42-1.17 (m, 16H), 1.16-1.04 (m, 1H), 1.01-0.76 (m, 4H).

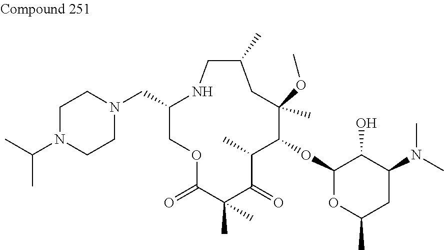

(3S,6R,8R,9R,10R)-9-(((2S,3R,4S,6R)-4-(dimethylamino)-3-hydroxy-6-methyltetrahydro-2H-pyran-2-yl)oxy)-3-((4-isopropylpiperazin-1-yl)methyl)-8-methoxy-6,8,10,12,12-pentamethyl-1-oxa-4-azacyclotridecane-11,13-dione

Prepared according to the methods of S3-2-I4-1-2 from S3-2-I13-1 to provide the title compound as a formate salt. MS (ESI+) m/z: 626.92 [M+H]+; ¹H NMR (400 MHz, Methanol-d4) δ 8.54 (s, 1H), 4.41 (d, 1H), 4.13-3.98 (m, 2H), 3.66 (m, 3H), 2.88 (s, 3H), 2.73 (m, 3H), 2.66 (m, 3H), 2.60-2.4 (m, 7H), 1.92 (m, 2H), 1.59 (m, 2H), 1.51 (s, 3H), 1.37-1.25 (m, 9H), 1.14 (d, 5H), 1.01 (d, 3H).

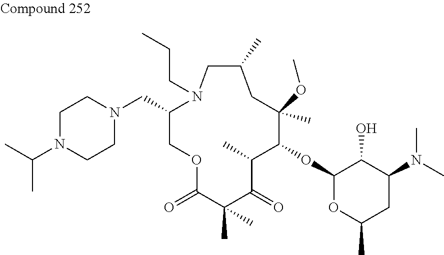

(3S,6R,8R,9R,10R)-9-(((2S,3R,4S,6R)-4-(dimethylamino)-3-hydroxy-6-methyltetrahydro-2H-pyran-2-yl)oxy)-3-((4-isopropylpiperazin-1-yl)methyl)-8-methoxy-6,8,10,12,12-pentamethyl-4-propyl-1-oxa-4-azacyclotridecane-11,13-dione

Prepared according to the methods of S3-2-I4-1-2 from S3-2-113-2 and acetone to provide the title compound as a free base. MS (ESI+) m/z: 669.27 [M+H]+; ¹H NMR (400 MHz, Methanol-d4) δ 4.58 (d, 1H), 4.36 (d, 1H), 4.03 (d, 1H), 3.87 (t, 1H), 3.64-3.51 (m, 2H), 3.27-3.22 (m, 1H), 2.70-2.53 (m, 3H), 2.63 (m, 7H), 2.36 (s, 9H), 2.16 (m, 3H), 1.77 (m, 2H), 1.53 (s, 3H), 1.47 (q, J=7.3 Hz, 2H), 1.35 (s, 3H), 1.31-1.21 (m, 9H), 1.10 (d, 5H), 0.96 (t, 3H), 0.90 (d, 3H).

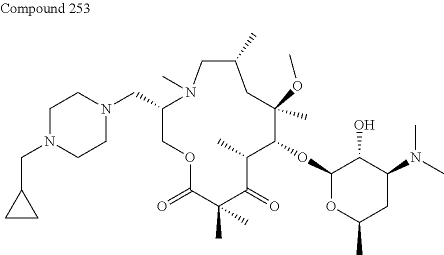

(3S,6R,8R,9R,10R)-3-((4-(cyclopropylmethyl)piperazin-1-yl)methyl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione

Prepared according to the methods of S3-2-I4-1-2 from S3-2-I13-2 and cyclopropanecarbaldehyde to provide the title compound as a formate salt. MS (ESI+) m/z: 653.5 [M+H]+; ¹H NMR (400 MHz, Methanol-d4) δ 8.52 (s, 2H), 4.45 (d, 1H), 4.20 (d, 1H), 3.72 (ddt, 1H), 3.53-3.34 (m, 3H), 3.02 (m, 8H), 2.80 (s, 14H), 2.54 (s, 1H), 2.07-1.98 (m, 1H), 1.58-1.44 (m, 4H), 1.39 (s, 5H), 1.33 (dd, 7H), 1.04 (m, 4H), 0.72-0.61 (m, 2H), 0.31 (t, J=5.1 Hz, H).

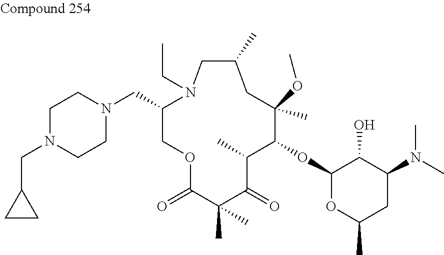

(3S,6R,8R,9R,10R)-3-((4-(cyclopropylmethyl)piperazin-1-yl)methyl)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-1-oxa-4-azacyclotridecane-11,13-dione

Prepared according to the methods S3-2-I4-1-2 from S3-1-I13-2 and cyclopropanecarbaldehyde to provide the title compound as a free base. MS (ESI+) m/z: 666.98 [M+H]+; ¹H NMR (400 MHz, Methanol-d4) δ 4.42 (d, 1H), 4.36 (d, 1H), 4.01 (d, 1H), 3.94 (d, 1H), 3.58 (m, 1H), 3.36 (d, 1H), 2.81 (s, 3H), 2.63 (s, 6H), 2.41 (s, 8H), 2.30 (d, 2H), 2.23 (d, 2H), 2.13 (s, 2H), 1.79 (d, 1H), 1.67 (s, 1H), 1.49 (s, 2H), 1.41 (s, 1H), 1.35 (s, 3H), 1.30-1.21 (m, 7H), 1.06 (q, 3H), 0.88 (d, 3H), 0.55 (d, 2H), 0.15 (d, 2H).

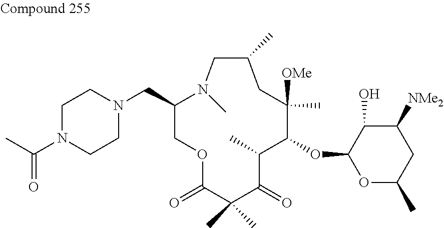

(3R,6R,8R,9R,10R)-3-((4-acetylpiperazin-1-yl)methyl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione

Prepared according to the methods of S3-4-I4-1-1 from S3-2-I7-1 and acetic anhydride to provide the title compound as a formate salt. MS (ESI+) m/z: 641.3 [M+H]+; ¹H NMR (400 MHz, Methanol-d4) δ 8.54 (br s, 1H), 4.47-4.39 (m, 1H), 3.75-3.49 (m, 6H), 3.38-3.34 (m, 1H), 3.18-2.74 (m, 8H), 2.71-2.33 (m, 16H), 2.09 (s, 3H), 1.91 (br dd, J=1.8, 9.2 Hz, 2H), 1.54 (br s, 3H), 1.47-1.18 (m, 16H), 1.14-0.86 (m, 4H).

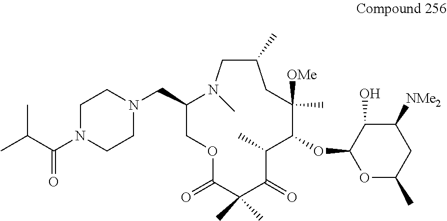

(3R,6R,8R,9R,10R)-9-(((2S,3R,4S,6R)-4-(dimethylamino)-3-hydroxy-6-methyltetrahydro-2H-pyran-2-yl)oxy)-3-((4-isobutyrylpiperazin-1-yl)methyl)-8-methoxy-4,6,8,10,12,12-hexamethyl-1-oxa-4-azacyclotridecane-11,13-dione

Prepared according to the methods of S3-4-I4-1-1 from S3-2-I7-1 and isobutyryl chloride to provide the title compound as a formate salt. MS (ESI+) m/z: 669.3 [M+H]+; ¹H NMR (400 MHz, Methanol-d4) δ 8.55 (br s, 0.3H), 4.69-4.47 (m, 1H), 4.44-4.28 (m, 2H), 4.15-4.05 (m, 1H), 4.04-3.95 (m, 1H), 3.74-3.51 (m, 6H), 3.28-3.16 (m, 2H), 2.99-2.79 (m, 4H), 2.71 (m, 1H), 2.65-2.27 (m, 16H), 2.03 (br d, 2H), 1.83-1.64 (m, 2H), 1.49 (br s, 3H), 1.40-1.19 (m, 14H), 1.09 (d, 7H), 0.92-0.81 (m, 3H).

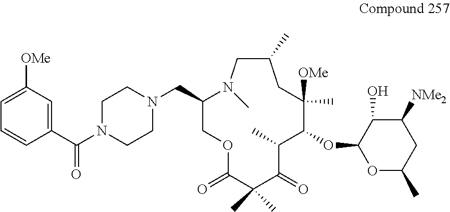

(3R,6R,8R,9R,10R)-9-(((2S,3R,4S,6R)-4-(dimethylamino)-3-hydroxy-6-methyltetrahydro-2H-pyran-2-yl)oxy)-8-methoxy-3-((4-(3-methoxybenzoyl)piperazin-1-yl)methyl)-4,6,8,10,12,12-hexamethyl-1-oxa-4-azacyclotridecane-11,13-dione

Prepared according to the methods of S3-4-I4-1-1 from S3-2-I7-1 and 3-methoxybenzoyl chloride to provide the title compound as a formate salt. MS (ESI+) m/z: 733.3 [M+H]+; ¹H NMR (400 MHz, Methanol-d4) δ 7.49-7.25 (m, 1H), 7.06 (br d, 1H), 7.00-6.92 (m, 2H), 4.43-4.31 (m, 2H), 4.11 (br t, 1H), 4.01 (br d, 1H), 3.84 (s, 3H), 3.78 (br d, 2H), 3.68 (m, 1H), 3.59 (m, 1H), 3.47 (br s, 2H), 3.29-3.12 (m, 2H), 2.87 (s, 3H), 2.71-2.59 (m, 3H), 2.51 (m, 3H), 2.42-2.24 (m, 10H), 2.12-1.99 (m, 2H), 1.76 (m, 2H), 1.50 (s, 2H), 1.36 (s, 3H), 1.33-1.18 (m, 10H), 1.12 (m, 2H), 0.88 (br d, 3H).

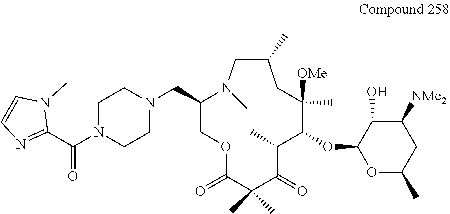

(3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-((4-(1-methyl-1H-imidazole-2-carbonyl)piperazin-1-yl)methyl)-1-oxa-4-azacyclotridecane-11,13-dione

Prepared according to the methods of S3-4-I4-1-1 from S3-2-I7-1 and 1-methyl-1H-imidazole-2-carbonyl chloride to provide the title compound as a formate salt. MS (ESI+) m/z: 707.3 [M+H]+; ¹H NMR (400 MHz, Methanol-d4) δ 7.22 (s, 1H), 7.03 (s, 1H), 4.65-4.26 (m, 4H), 4.23-4.05 (m, 2H), 4.04-3.93 (m, 1H), 3.87-3.69 (m, 6H), 3.63 (dt, J=5.5, 10.7 Hz, 3H), 3.24-3.07 (m, 2H), 3.04-2.76 (m, 5H), 2.75-2.55 (m, 4H), 2.54-2.18 (m, 10H), 2.04 (br d, 2H), 1.91-1.67 (m, 3H), 1.63-1.2 (m, 14H), 1.17-0.79 (m, 4H).

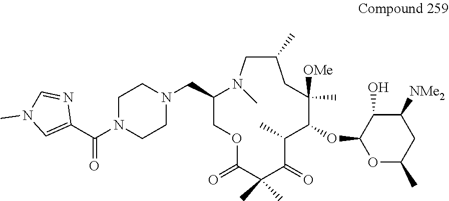

(3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-((4-(1-methyl-1H-imidazole-4-carbonyl)piperazin-1-yl)methyl)-1-oxa-4-azacyclotridecane-11,13-dione

Prepared according to the methods of S3-4-I4-1-1 from S3-2-I7-1 and 1-methyl-1H-imidazole-4-carbonyl chloride to provide the title compound as a formate salt. MS (ESI+) m/z: 707.3 [M+H]+; ¹H NMR (400 MHz, Methanol-d4) δ 8.55 (s, 0.4H), 7.64 (s, 1H), 7.54 (s, 1H), 4.40 (m, 2H), 4.17-3.55 (m, 11H), 3.24-3.12 (m, 1H), 3.11-2.72 (m, 5H), 2.70-2.17 (m, 16H), 2.03 (m, 2H), 1.80 (m, 2H), 1.64-1.18 (m, 17H), 1.17-0.75 (m, 4H).

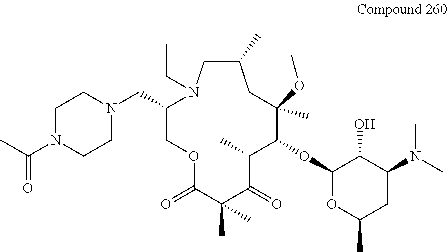

(3S,6R,8R,9R,10R)-3-((4-acetylpiperazin-1-yl)methyl)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-1-oxa-4-azacyclotridecane-11,13-dione

Prepared according to the methods of S3-4-I4-1-1 from S3-2-I13-2 and acetic anhydride to provide the title compound as a free base. MS (ESI+) m/z: 655.08 [M+H]+, 676.98 [M+Na]+; ¹H NMR (400 MHz, Methanol-d4) δ 4.49 (d, 1H), 4.37 (d, 1H), 4.03 (d, 1H), 3.95 (t, 1H), 3.56 (m, 6H), 2.82 (s, 3H), 2.75 (m, 2H), 2.58 (m, 1H), 2.55-2.47 (m, 1H), 2.42 (s, 7H), 2.32 (m, 2H), 2.25 (d, 1H), 2.19 (d, 1H), 2.15 (s, 1H), 2.09 (s, 3H), 1.79 (m, 1H), 1.68 (s, 1H), 1.51 (s, 3H), 1.43 (s, 1H), 1.37 (s, 3H), 1.28 (dt, 8H), 1.06 (t, 3H), 0.89 (d, 3H).

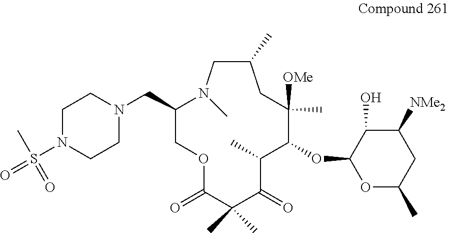

(3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-((4-(methylsulfonyl)piperazin-1-yl)methyl)-1-oxa-4-azacyclotridecane-11,13-dione

Prepared according to the methods of S3-4-I4-1-1 from S3-2-I7-1 and methanesulfonyl chloride to provide the title compound as a formate salt. MS (ESI+) m/z: 677.2 [M+H]+; ¹H NMR (400 MHz, Methanol-d4) δ 8.55 (br s, 0.5H), 4.46-4.27 (m, 2H), 4.17-3.94 (m, 2H), 3.72-3.52 (m, 2H), 3.27-3.08 (m, 5H), 2.99-2.76 (m, 7H), 2.25 (br d, 17H), 2.11-1.95 (m, 2H), 1.88-1.66 (m, 2H), 1.57-1.44 (m, 3H), 1.42-1.19 (m, 13H), 1.17-1.03 (m, 2H), 0.96-0.78 (m, 3H).

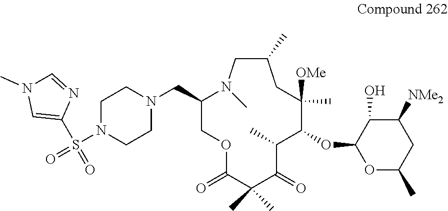

(3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-((4-((1-methyl-1H-imidazol-4-yl)sulfonyl)piperazin-1-yl)methyl)-1-oxa-4-azacyclotridecane-11,13-dione

Prepared according to the methods of S3-4-I4-1-1 from S3-2-I7-1 and propane-2-sulfonyl chloride to provide the title compound as a formate salt. MS (ESI+) m/z: 743.3 [M+H]+; ¹H NMR (400 MHz, Methanol-d4) δ 8.55 (br s, 0.3H), 7.79 (s, 1H), 7.72 (s, 1H), 4.48-4.16 (m, 2H), 4.11-3.91 (m, 2H), 3.81 (s, 3H), 3.70-3.52 (m, 2H), 3.25-2.98 (m, 6H), 2.96-2.74 (m, 4H), 2.71-2.52 (m, 4H), 2.50-2.19 (m, 12H), 2.01 (br d, J=11.0 Hz, 2H), 1.88-1.61 (m, 2H), 1.59-1.16 (m, 16H), 1.15-0.74 (m, 4H).

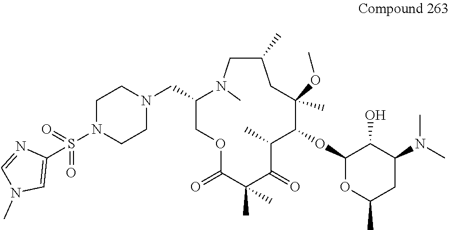

(3S,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-((4-((1-methyl-1H-imidazol-4-yl)sulfonyl)piperazin-1-yl)methyl)-1-oxa-4-azacyclotridecane-11,13-dione

Prepared according to the methods of S3-4-I4-1-1 from S3-2-I13-1 and 1-methyl-1H-imidazole-4-sulfonyl chloride to provide the title compound as a formate salt. MS (ESI+) m/z: 742.96 [M+H]+, 765.9 [M+Na]+; ¹H NMR (400 MHz, Methanol-d4) δ 8.54 (s, 1H), 7.78 (d, 1H), 7.72 (d, 1H), 4.63-4.47 (m, 1H), 4.39 (d, 1H), 3.80 (s, 3H), 3.65 (m, 1H), 3.11 (s, 5H), 2.77 (s, 2H), 2.61 (s, 9H), 1.90 (m, 1H), 1.52 (s, 3H), 1.28 (m, 13H).

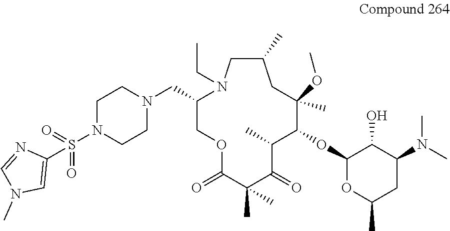

(3S,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-3-((4-((1-methyl-1H-imidazol-4-yl)sulfonyl)piperazin-1-yl)methyl)-1-oxa-4-azacyclotridecane-11,13-dione

Prepared according to the methods of S3-4-I4-1-1 from S3-2-I13-2 and 1-methyl-1H-imidazole-4-sulfonyl chloride to provide the title compound as a free base. MS (ESI+) m/z: 757.03 [M+H]+, 778.83 [M+Na]+; ¹H NMR (400 MHz, Methanol-d4) δ 7.78 (d, 1H), 7.71 (d, 1H), 4.39 (s, 1H), 4.34 (d, 1H), 4.00 (d, 1H), 3.87 (t, 1H), 3.80 (s, 3H), 3.61-3.42 (m, 2H), 3.26-3.15 (m, 2H), 3.09 (s, 4H), 2.78 (s, 3H), 2.73-2.54 (m, 4H), 2.44-2.37 (m, 3H), 2.35 (s, 6H), 2.23-2.04 (m, 3H), 1.76 (d, 1H), 1.64 (s, 1H), 1.48 (s, 3H), 1.32 (s, 3H), 1.29-1.19 (m, 9H), 1.03 (t, 4H), 0.86 (d, 3H).

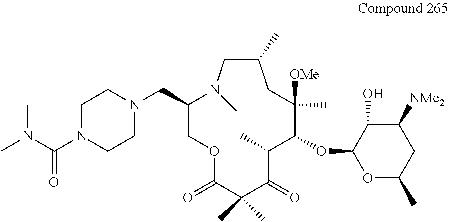

4-(((3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-11,13-dioxo-1-oxa-4-azacyclotridecan-3-yl)methyl)-N,N-dimethylpiperazine-1-carboxamide

Prepared according to the methods of S3-4-I4-1-1 from S3-2-I7-1 to provide the title compound as a formate salt. MS (ESI+) m/z: 670.3 [M+H]+; ¹H NMR (400 MHz, Methanol-d4) δ 8.55 (br s, 0.5H), 4.41 (br d, J=7.1 Hz, 1H), 4.10 (br s, 1H), 3.70-3.56 (m, 2H), 3.25 (br t, 5H), 2.93-2.84 (m, 12H), 2.70-2.17 (m, 18H), 2.14-1.90 (m, 1H), 1.85-1.60 (m, 2H), 1.52 (br s, 3H), 1.37-1.26 (m, 14H), 0.92 (br s, 3H)=8.55 (br s, 1H), 4.41 (br d, 1H), 4.10 (br s, 1H), 3.70-3.56 (m, 2H), 3.25 (br t, 5H), 2.93-2.84 (m, 12H), 2.70-2.17 (m, 18H), 2.14-1.90 (m, 1H), 1.85-1.60 (m, 2H), 1.52 (br s, 3H), 1.37-1.26 (m, 14H), 0.92 (br s, 3H).

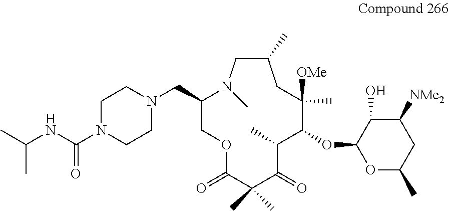

4-(((3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-11,13-dioxo-1-oxa-4-azacyclotridecan-3-yl)methyl)-N-isopropylpiperazine-1-carboxamide

Prepared according to the methods of S3-4-I4-1-1 from S3-2-17-1 and 2-isocyanatopropane to provide the title compound as a formate salt. MS (ESI+) m/z: 684.3 [M+H]+; ¹H NMR (400 MHz, Methanol-d4) δ 8.55 (br s, 0.2H), 4.46-4.26 (m, 2H), 4.22-3.96 (m, 2H), 3.88 (td, 1H), 3.82-3.52 (m, 2H), 3.38 (br s, 4H), 3.28-3.11 (m, 2H), 3.08-2.65 (m, 5H), 2.65-2.18 (m, 16H), 2.03 (br dd, 2H), 1.86-1.64 (m, 2H), 1.61-1.43 (m, 3H), 1.42-1.21 (m, 13H), 1.19-1.04 (m, 8H), 1.02-0.76 (m, 3H).

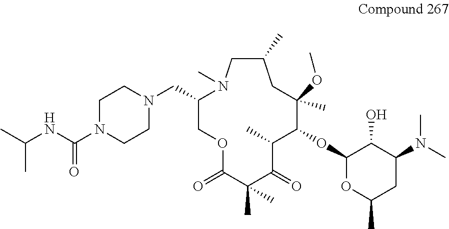

4-(((3S,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-11,13-dioxo-1-oxa-4-azacyclotridecan-3-yl)methyl)-N-isopropylpiperazine-1-carboxamide

Prepared according to the methods of S3-4-I4-1-1 from S3-1-I13-1 and 2-isocyanatopropane to provide the title compound as a formate salt. MS (ESI+) m/z: 684.04 [M+H]+; ¹H NMR (400 MHz, Methanol-d4) δ 8.53 (s, 1H), 4.44 (d, 1H), 4.21 (s, 2H), 3.88 (p, 1H), 3.71 (m, 1H), 3.49-3.35 (m, 5H), 3.03 (s, 3H), 2.81 (d, 2H), 2.75 (s, 6H), 2.52 (s, 4H), 1.99 (ddd, 1H), 1.49 (m, 4H), 1.39 (s, 5H), 1.33 (t, 6H), 1.13 (d, 6H), 1.05 (s, 2H).

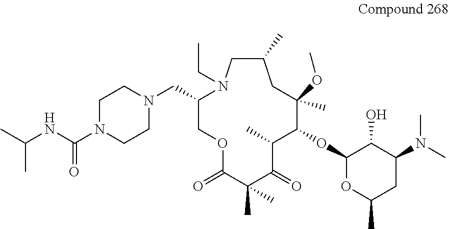

4-(((3S,6R,8R,9R,10R)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-11,13-dioxo-1-oxa-4-azacyclotridecan-3-yl)methyl)-N-isopropylpiperazine-1-carboxamide

Prepared according to the methods of S3-4-I4-1-1 from S3-1-I13-2 and 2-isocyanatopropane to provide the title compound as a free base. MS (ESI+) m/z: 697.98 [M+H]+; ¹H NMR (400 MHz, Methanol-d4) δ 4.48 (d, 1H), 4.37 (d, 1H), 4.03 (d, 1H), 3.99-3.84 (m, 2H), 3.67-3.47 (m, 2H), 3.38 (t, 4H), 3.26 (m, 1H), 2.82 (s, 3H), 2.71 (m, 2H), 2.51 (m, 2H), 2.38 (s, 7H), 2.37-2.28 (m, 4H), 2.28-2.18 (m, 1H), 2.18-2.07 (m, 2H), 1.78 (d, 1H), 1.68 (m, 1H), 1.51 (s, 3H), 1.36 (s, 3H), 1.34-1.23 (m, 9H), 1.14 (d, 6H), 1.06 (t, 4H), 0.89 (d, 3H).

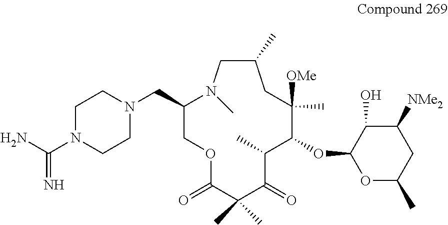

4-(((3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-11,13-dioxo-1-oxa-4-azacyclotridecan-3-yl)methyl)piperazine-1-carboximidamide

A solution of S3-2-I7-1 (55 mg, 78 μmol) in DMF was treated with triethylamine, N,N′-Bis(tert-butoxycarbonyl)-S-methylisothiourea and mercuric chloride at rt for 2.5 h. The reaction mixture was filtered, concentrated and purified by column chromatography on silica gel to give 25 mg (34%) of the Boc-protected intermediate. This material was dissolved in DCM and was treated with TFA at rt for 30 min. The resulting solution was partitioned between DCM and satd aq NaHCO3 and the aqueous phase was extracted with DCM. The combined organic phases were dried, filtered and concentrated. The residue was dissolved in MeOH and was heated at 55° C. for 13 h, then concentrated and purified by HPLC (elution with MeCN-water-0.1% formic acid) to give 4.7 mg (26%) of the title compound as a formate salt. MS (ESI+) m/z: 641.3 [M+H]+; ¹H NMR (400 MHz, Methanol-d4) δ 8.56 (br s, 3H), 4.59 (br s, 2H), 4.45 (br d, 2H), 4.28-4.02 (m, 2H), 3.72-3.65 (m, 1H), 3.54-3.46 (m, 5H), 3.41-3.34 (m, 2H), 3.13 (m, 2H), 3.03-2.87 (m, 4H), 2.65 (br d, 17H), 1.97-1.90 (m, 2H), 1.53 (br s, 3H), 1.40-1.26 (m, 15H), 0.95 (br s, 4H).

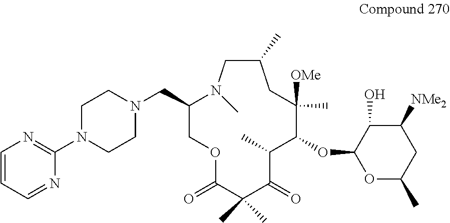

(3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-((4-(pyrimidin-2-yl)piperazin-1-yl)methyl)-1-oxa-4-azacyclotridecane-11,13-dione

A solution of S3-2-I7-1 (38 mg, 54 μmol) in DMSO was treated with diisopropylethylamine and 2-chloropyrimidine and heated at 50° C. for 4 h, then allowed to cool. The resulting solution was partitioned between DCM and satd aq NaHCO₃ and the aqueous phase was extracted with DCM. The combined organic phases were dried, filtered and concentrated to give 45 mg of crude product. The residue was dissolved in MeOH and was heated at 55° C. for 13 h, then concentrated and purified by HPLC (elution with MeCN-water-0.1% formic acid) to give 8.1 mg (21%) of the title compound as a formate salt. MS (ESI+) m/z: 677.4 [M+H]+; ¹H NMR (400 MHz, Methanol-d4) δ 8.56 (s, 0.3H), 8.39-8.27 (m, 2H), 6.59 (br s, 1H), 4.40 (br d, J=7.1 Hz, 2H), 4.23-3.94 (m, 2H), 3.80 (br s, 8H), 2.97-2.21 (m, 22H), 2.14-1.97 (m, 2H), 1.85-1.65 (m, 2H), 1.56-1.20 (m, 17H), 1.16-0.76 (m, 4H).

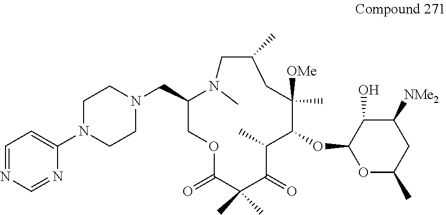

(3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-((4-(pyrimidin-4-yl)piperazin-1-yl)methyl)-1-oxa-4-azacyclotridecane-11,13-dione

Prepared as described for compound 270 from S3-2-17-1 and 4-chloropyrimidine to give the title compound as a formate salt. MS (ESI+) m/z: 677.4 [M+H]+; ¹H NMR (400 MHz, Methanol-d4) δ 8.55 (m, 0.2H), 8.45 (s, 1H), 8.11 (br d, 1H), 6.77 (br d, 1H), 4.41 (br d, 2H), 4.18-3.93 (m, 2H), 3.80-3.53 (m, 7H), 3.02-2.23 (m, 22H), 2.06 (br s, 1H), 1.88-1.65 (m, 2H), 1.60-0.75 (m, 22H).

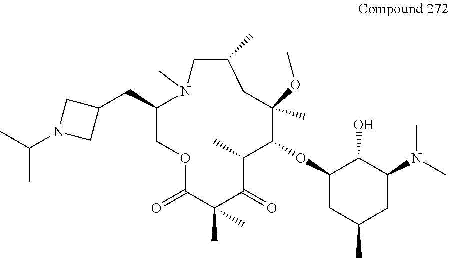

(3R,6R,8R,9R,10R)-9-(((2S,3R,4S,6R)-4-(dimethylamino)-3-hydroxy-6-methyltetrahydro-2H-pyran-2-yl)oxy)-3-((1-isopropylazetidin-3-yl)methyl)-8-methoxy-4,6,8,10,12,12-hexamethyl-1-oxa-4-azacyclotridecane-11,13-dione

Prepared according to the methods S3-2-I4-1-2 from 114 and acetone to provide the title compound. MS (ESI+) m/z: 612.41 [M+H]+; ¹H NMR (400 MHz, CDCl₃) δ 8.53 (s, 4H), 4.62 (dd, 1H), 4.55-4.45 (m, 2H), 4.44 (d, 1H), 4.12 (d, 1H), 3.85 (m, 2H), 3.78-3.64 (m, 2H), 3.43 (dd, 2H), 3.39-3.33 (m, 1H), 3.25 (t, 1H), 3.21-3.11 (m, 4H), 3.11-2.93 (m, 4H), 2.86 (s, 3H), 2.78 (s, 7H), 2.74-2.65 (m, 1H), 2.37 (m, 1H), 2.07-1.96 (m, 1H), 1.79 (dt, 2H), 1.56 (s, 3H), 1.50 (m, 2H), 1.40 (d, 7H), 1.36-1.23 (m, 14H), 1.11 (d, 3H).

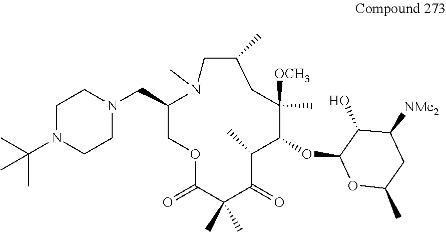

(3R,6R,8R,9R,10R)-3-((4-(tert-butyl)piperazin-1-yl)methyl)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-1-oxa-4-azacyclotridecane-11,13-dione

Prepared according to the methods of S2-2-I3-1 from 115 to provide the title compound as a formate salt. MS (ESI+) m/z: 655.02 [M+H]+. ¹H NMR (400 MHz, Methanol-d₄) δ 8.52 (s, 2.97H, HCO2H), 4.65 (d, 1H), 4.46 (d, 1H), 4.36 (dd, 1H), 4.25 (d, 1H), 3.84 (s, 1H), 3.73 (dtd, 1H), 3.46 (dd, 2H), 3.37 (ddd, 1H), 3.17 (d, 5H), 3.03 (s, 4H), 2.80 (s, 14H), 2.66 (dd, 1H), 2.18 (s, 1H), 2.07-1.96 (m, 1H), 1.70 (s, 2H), 1.58-1.46 (m, 4H), 1.44-1.26 (m, 22H), 1.04 (d, 3H).

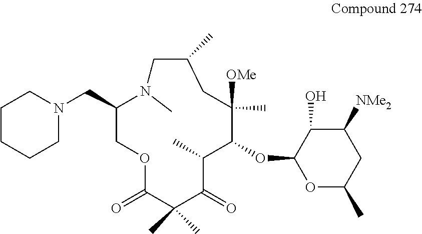

(3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(piperidin-1-ylmethyl)-1-oxa-4-azacyclotridecane-11,13-dione

Prepared according to the methods of S2-2-I3-1 from 116. White solid, formic acid salt. MS (ESI+) m/z: 597.93 [M+H]+. ¹H NMR (400 MHz, Methanol-d₄) δ 8.51 (s, 2H), 4.55-4.41 (m, 2H), 4.37 (dd, 1H), 4.27 (d, 1H), 3.91 (d, 1H), 3.78-3.66 (m, 1H), 3.55-3.39 (m, 2H), 3.40-3.33 (m, 1H), 3.24 (dd, 1H), 3.06 (d, 1H), 3.02 (s, 3H), 2.67-2.43 (m, 5H), 2.21 (s, 1H), 2.07-1.98 (m, 1H), 1.78 (d, 1H), 1.71-1.56 (m, 8H), 1.56-1.46 (m, 3H), 1.40 (s, 3H), 1.39 (s, 3H), 1.34 (d, 3H), 1.33 (d, 3H), 1.05 (d, 3H).

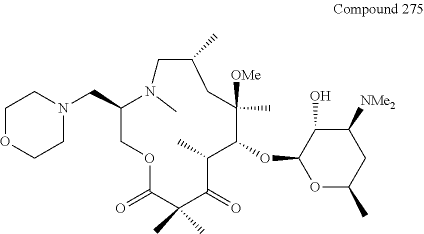

(3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-(morpholinomethyl)-1-oxa-4-azacyclotridecane-11,13-dione

Prepared according to the methods of S2-2-I3-1 from 117 to provide the title compound as a formate salt. MS (ESI+) m/z: 599.91 [M+H]+. ¹H NMR (400 MHz, Methanol-d₄) δ 8.53 (s, 2H), 4.59 (d, 1H), 4.45 (d, 1H), 4.35 (d, 1H), 4.26 (d, 1H), 3.88 (s, 1H), 3.77-3.60 (m, 5H), 3.55-3.34 (m, 2H), 3.11-2.96 (m, 4H), 2.84 (s, 3H), 2.79-2.63 (m, 8H), 2.63-2.42 (m, 5H), 2.20 (s, 1H), 2.07-1.92 (m, 1H), 1.70 (s, 2H), 1.58 (s, 3H), 1.49 (td, 1H), 1.40 (d, 6H), 1.33 (t, 6H), 1.05 (d, 3H).

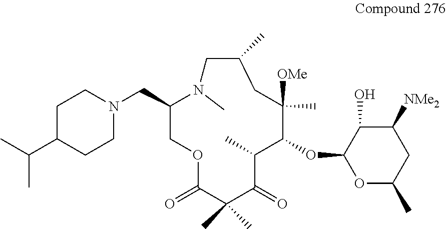

(3R,6R,8R,9R,10R)-9-(((2S,3R,4S,6R)-4-(dimethylamino)-3-hydroxy-6-methyltetrahydro-2H-pyran-2-yl)oxy)-3-((4-isopropylpiperidin-1-yl)methyl)-8-methoxy-4,6,8,10,12,12-hexamethyl-1-oxa-4-azacyclotridecane-11,13-dione

Prepared according to the methods of S2-2-I3-1 from 118 to provide the title compound as a formate salt. MS (ESI+) m/z: 640.01 [M+H]+. ¹H NMR (400 MHz, Methanol-d₄) δ 8.56 (s, 1H), 4.52-4.34 (m, 2H), 4.34-4.21 (m, 1H), 4.18 (d, 1H), 3.65 (ddd, 2H), 3.54 (q, 1H), 3.42-3.33 (m, 1H), 3.09-2.87 (m, 7H), 2.80-2.60 (m, 5H), 2.54 (s, 6H), 2.47 (dd, 2H), 2.17-1.94 (m, 3H), 1.88 (ddd, 1H), 1.83-1.64 (m, 3H), 1.56 (s, 3H), 1.49-1.40 (m, 1H), 1.37 (d, 7H), 1.32 (d, 3H), 1.28 (d, 6H), 1.05 (d, 2H), 0.99 (d, 3H), 0.90 (d, 6H).

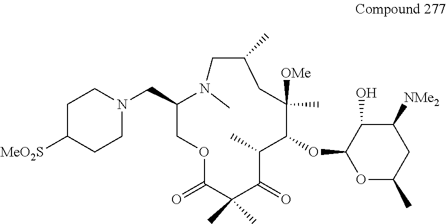

(3R,6R,8R,9R,10R)-9-(((2S,3R,4S,6R)-4-(dimethylamino)-3-hydroxy-6-methyltetrahydro-2H-pyran-2-yl)oxy)-8-methoxy-4,6,8,10,12,12-hexamethyl-3-((4-(methylsulfonyl)piperidin-1-yl)methyl)-1-oxa-4-azacyclotridecane-11,13-dione. Prepared according to the methods of S2-2-I3-1 from 119 to provide the title compound as a formate salt. MS (ESI+) m/z: 675.92 [M+H]+. ¹H NMR (400 MHz, Methanol-d₄) δ 8.58 (s, 1H), 4.64-4.48 (m, 1H), 4.44 (d, 1H), 4.40-4.26 (m, 1H), 4.27-4.14 (m, 1H), 3.75-3.60 (m, 1H), 3.59-3.43 (m, 1H), 3.44-3.35 (m, 1H), 3.25-3.03 (m, 5H), 3.00 (s, 3H), 2.93 (s, 3H), 2.86-2.71 (m, OH), 2.68 (s, 6H), 2.61-2.45 (m, 1H), 2.29-2.03 (m, 5H), 2.02-1.89 (m, 1H), 1.78 (ddt, 4H), 1.57 (s, 3H), 1.52-1.42 (m, 1H), 1.39 (d, 5H), 1.36-1.24 (m, 8H), 1.02 (d, 3H).

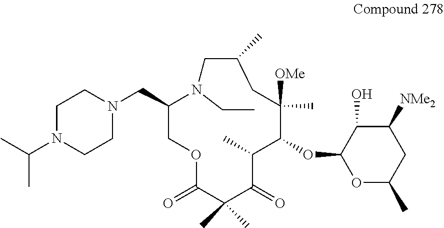

(3R,6R,8R,9R,10R)-9-(((2S,3R,4S,6R)-4-(dimethylamino)-3-hydroxy-6-methyltetrahydro-2H-pyran-2-yl)oxy)-4-ethyl-3-((4-isopropylpiperazin-1-yl)methyl)-8-methoxy-6,8,10,12,12-pentamethyl-1-oxa-4-azacyclotridecane-11,13-dione

Prepared according to the methods of S2-2-I3-1 from 120 to provide the title compound as a formate salt. MS (ESI+) m/z: 654.98 [M+H]+. ¹H NMR (400 MHz, Methanol-d₄) δ 4.36 (d, J=7.3 Hz, 1H), 4.30 (d, J=11.5 Hz, 1H), 4.01-3.93 (m, 2H), 3.69 (p, J=6.9 Hz, 1H), 3.63-3.50 (m, 1H), 3.26 (dd, J=10.2, 7.3 Hz, 2H), 3.21-3.11 (m, 1H), 2.80 (s, 3H), 2.75-2.52 (m, 11H), 2.51-2.36 (m, 4H), 2.33 (s, 6H), 2.28 (d, J=14.2 Hz, 2H), 2.07-1.98 (m, 1H), 1.75 (ddd, J=12.7, 4.3, 1.9 Hz, 1H), 1.52 (s, 3H), 1.34 (s, 3H), 1.32-1.25 (m, 7H), 1.23 (d, J=6.2 Hz, 3H), 1.14-1.03 (m, 9H), 1.00 (dd, J=13.8, 8.8 Hz, 2H), 0.85 (d, J=6.5 Hz, 3H).

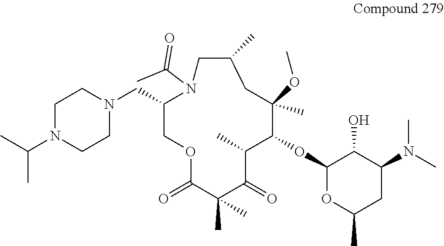

(3S,6R,8R,9R,10R)-4-acetyl-9-(((2S,3R,4S,6R)-4-(dimethylamino)-3-hydroxy-6-methyltetrahydro-2H-pyran-2-yl)oxy)-3-((4-isopropylpiperazin-1-yl)methyl)-8-methoxy-6,8,10,12,12-pentamethyl-1-oxa-4-azacyclotridecane-11,13-dione

Prepared according to the methods of S8-8-I12-1 from 113 and acetic anhydride to provide the title compound as a formate salt. MS (ESI+) m/z: 669.08 [M+H]+, 691.08 [M+Na]+; ¹H NMR (400 MHz, Methanol-d4) δ 8.56 (s, 1H), 4.34 (d, 2H), 4.34 (m, 1H), 3.97 (dd, 2H), 3.57 (m, 1H), 3.25-3.18 (m, 2H), 3.05 (m, 1H), 2.86 (s, 1H), 2.78 (s, 3H), 2.68-2.43 (m, 12H), 2.34 (s, 6H), 2.13 (d, 4H), 1.76 (d, 2H), 1.45-1.35 (m, 7H), 1.32 (d, 2H), 1.29-1.18 (m, 10H), 1.09 (dd, 6H), 0.99 (d, 2H), 0.92 (d, 2H).

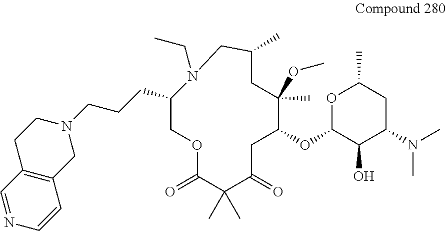 [SMILES: CCN1C[C@H](C)C[C@@](C)(OC)[C@H](O[C@@H]2O[C@H](C)C[C@H](N(C)C)[C@H]2O)CC(=O)C(C)(C)C(=O)OC[C@@H]1CCCN1CCc2cnccc2C1]

(3S,6R,8R,9R,10R)-3-(3-(3,4-dihydro-2,6-naphthyridin-2(1H)-yl)propyl)-9-(((2S,3R,4S,6R)-4-(dimethylamino)-3-hydroxy-6-methyltetrahydro-2H-pyran-2-yl)oxy)-4-ethyl-8-methoxy-6,8,10,12,12-pentamethyl-1-oxa-4-azacyclotridecane-11,13-dione

Prepared according to the methods of S6-3-I1-1 from S2-1-I1-2 and 1,2,3,4-tetrahydro-2,6-naphthyridine to provide the title compound as a formate salt. MS (ESI+) m/z: 689.3 [M+H]+; ¹H NMR (400 MHz, Methanol-d4) δ 8.40 (s, 2.6H), 8.33 (s, 1H), 8.26 (d, 1H), 7.17 (d, 1H), 4.66-4.57 (m, 1H), 4.52-4.40 (m, 2H), 4.27 (br d, 1H), 4.03 (br d, 1H), 3.81-3.67 (m, 3H), 3.63-3.50 (m, 1H), 3.49-3.34 (m, 3H), 3.25-3.07 (m, 2H), 3.07-2.94 (m, 5H), 2.92-2.77 (m, 9H), 2.69 (br t, 2H), 2.17 (br d, 1H), 2.08-2.01 (m, 1H), 1.95 (br d, 1H), 1.88-1.70 (m, 3H), 1.63 (br d, 1H), 1.58-1.50 (m, 1H), 1.49-1.44 (m, 3H), 1.44-1.27 (m, 16H), 1.06 (br d, 3H).

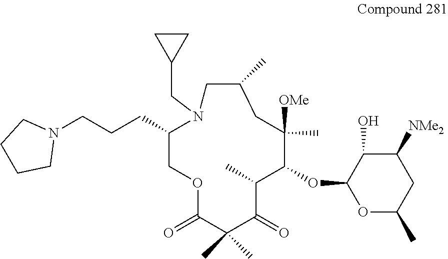

(3S,6R,8R,9R,10R)-4-(cyclopropylmethyl)-9-(((2S,3R,4S,6R)-4-(dimethylamino)-3-hydroxy-6-methyltetrahydro-2H-pyran-2-yl)oxy)-8-methoxy-6,8,10,12,12-pentamethyl-3-(3-(pyrrolidin-1-yl)propyl)-1-oxa-4-azacyclotridecane-11,13-dione

Prepared according to the methods of S6-3-I1-1 from S2-1-I1-5 and pyrrolidine to provide the title compound. MS (ESI+) m/z: 652.29 [M+H]+; ¹H NMR (400 MHz, Methanol-d4) δ 8.52 (s, 3H), 4.44 (d, 1H), 4.35-4.21 (m, 1H), 3.76-3.68 (m, 1H), 3.44 (dd, 1H), 3.36 (dd, 1H), 3.29-3.20 (m, 3H), 3.19-2.98 (m, 5H), 2.78 (s, 6H), 2.09-1.97 (m, 5H), 1.49 (s, 3H), 1.41-1.36 (m, 4H), 1.35-1.29 (m, 7H), 1.11-0.82 (m, 5H), 0.56-0.33 (m, 2H).

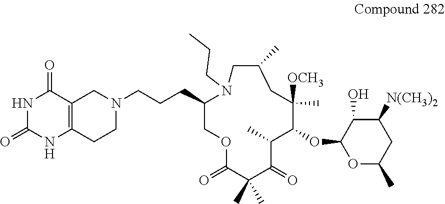

(3R,6R,8R,9R,10R)-9-(((2S,3R,4S,6R)-4-(dimethylamino)-3-hydroxy-6-methyltetrahydro-2H-pyran-2-yl)oxy)-3-(3-(2,4-dioxo-1,3,4,5,7,8-hexahydropyrido[4,3-d]pyrimidin-6(2H)-yl)propyl)-8-methoxy-6,8,10,12,12-pentamethyl-4-propyl-1-oxa-4-azacyclotridecane-11,13-dione

Prepared according to the methods of S6-3-I1-1-1 from (2S,3R,4S,6R)-4-(dimethylamino)-2-(((3R,6R,8R,9R,10R)-8-methoxy-6,8,10,12,12-pentamethyl-11,13-dioxo-3-(3-oxopropyl)-4-propyl-1-oxa-4-azacyclotridecan-9-yl)oxy)-6-methyltetrahydro-2H-pyran-3-yl benzoate and hexahydropyrido[4,3-d]pyrimidine-2,4(1H,3H)-dione.

### Biological Activity

The biological activity of the compounds disclosed herein was tested by the Cystic Fibrosis Foundation.

Example 1: Nanoluc Assay

FRT cells expressing CFTR β-globin fusion proteins (W134X) are cultured in Coon's F-12 Medium supplemented with 5% fetal bovine serum and 100 units/mL penicillin-streptomycin. Cells are kept in a humidified, 5% CO₂ atmosphere at 37° C. Cells are transfected with 0.5 ug/mL of the described construct, and 48 hours later, the levels of luciferase are measured with the luciferase assay lit (Promega, USA), according to the manufacturer's instructions.

A NanoLuc luciferase reporter plasmid was developed to target the identification of both readthrough and NMD modulators by using the addition of a nonsense mutation (W134X) in the Nanoluc region to test for readthrough as well as upstream of a β-globin intron to test for NMD attenuation (Figure X). The G418 used in experiments was commercial G418-sulfate (Gibco, #10131-027). Following normalization, the differences in luciferase activities reflect the frequency of stop codon readthrough.
Panel 1: Readthrough was observed when cells were treated with various amounts of Compounds 95, 101, 113, 139, and 161, alone or in combination with 100 uM G418.
Panel 2: Readthrough was observed when cells were treated with various amounts of Compounds 95, 101, 113, 139, and 161, alone or in combination with alone or in combination with 100 uM G418 alone or in combination with 50 uM G418.

### Full Method

### Cell Culture

FRT cells expressing CFTR β-globin fusion proteins (W134X) are cultured at 37° C. and 5% CO2 in BioChrom Coon's F-12 Medium (Cedar Lane Labs, #F0855) with 5% fetal bovine serum (Gibco, #26140-079), 1% penicillin-streptomycin (Gibco, #15140-122), L-glutamine (Gibco, #25030) and 200 μg/mL Zeocin (Life Technology, #R25005).

### HTS

Cells are removed from −80° C. freezers, thawed, and pelleted at 1000 rpm for 5 min at room temperature. The cells are resuspended in FRT growth media. A multidrop reagent dispenser (Thermo Fisher Scientific, #22-387-053) is used to plate cells (25,000 cells/well, 50 μL/well) into 384-well assay plates (Corning, #3570BC). The plates are centrifuged at 500 rpm for 2 min at room temperature and incubated for 48 h at 37° C. and 5% CO₂. before compound administration of 5 μL/well, to yield a final assay volume of 55 μL/well. The cells are incubated for 48 h at 37° C. and 5% CO₂.

For the W134X screening, the medium is aspirated and replaced with 16 μL of Opti-MEM medium (Life Technology, #11058), without FBS, buffered with HEPES (Gibco, #15630-080). 4 μL of Nano-Glo Live Cell Reagent (Promega, #N2013) is added and luminescence was read using an integration time of 0.1-2 seconds.

Example 2: RDEB Assay Rational, Methodology and Results

### 1. Collagen Type VII Immunoblotting Assay

### Rationale

To assess the ability of compounds in inducing the expression of full-length Collagen VII protein in two RDEB patient Fibroblasts (R578X/R578X or R613X/R1683X) or keratinocytes (R2814X/R2814X, R2610X/R2610X or Q251X/Q251X) after 48 hours of treatment.

### Methodology

This method has been described in detail by Cogan and colleagues (Cogan et al., Molecular therapy, vol. 22,10 (2014): 1741-52). Cells were plated at 70-80% confluency on 6-well plates. On the following day, media were replaced with fresh media and cells were untreated or treated with test compounds (tested range: 10-60 μM, see table 3 for details) or gentamicin (positive control; 200 μg/ml or 400 μg/ml). At 24 hours, the media was replaced with fresh media and compound. After 48 hours of treatment, cells were lysed and subjected to immunoblot analysis using a polyclonal antibody to type VII collagen and a monoclonal antibody to β-Actin (loading control). ImageJ was used to quantify the intensity of the specific bands and all samples were normalized to β-Actin. Resultant densitometry values were expressed relative to β-Actin and then expressed as a percentage relative to non-treated cells.

The ability of the test compounds in inducing readthrough of Collagen VII protein was assessed in 4 different RDEB derived patient cells using two different cell types (fibroblasts and keratinocytes). The average readthrough activity is expressed as a percentage of activity relative to gentamicin: +=0-33% of gentamicin, ++=34-66% of gentamicin, and +++=>66% of gentamicin.

| | Conc. at | Cell type, Mutation and Readthrough Level | | | | |
|---|---|---|---|---|---|---|
| | max | Keratinocyte | | | Fibroblast | |
| Cmpd | response | R2814X/ | Q251X/ | R2610X/ | R578X/ | R613X/ |
| No. | (μM) | R2814X | Q251X | R2610X | R578X | R1683X |
| 198 | 60 | +++ | +++ | +++ | ++ | +++ |
| 60 | 50 | | | | ++ | +++ |
| 179 | 25 | | +++ | | + | +++ |
| 241 | 60 | +++ | +++ | +++ | + | +++ |

### 2. Fibroblast Migration Assay

### Rationale

RDEB patient fibroblasts have a hypermotility phenotype relative to fibroblasts derived from normal subjects. This cellular phenotype is thought to be linked to the inability of RDEB fibroblasts to attach to the growth substrate due to a lack of Collagen VII (Chen et al., Nature genetics vol 32,4 (2002): 670-5; Cogan et al., 2014). We therefore test the effect of test compounds in reducing/rescuing the hypermotility phenotype by induction of Collagen VII nonsense mutation readthrough.

### Methodology

This method has been described in detail by Cogan and colleagues (Woodley et al., Journal of celular physiology vol. 136,1 (1988): 140-6; Chen et al., 2002; and Cogan et al., 2014). Briefly, R578X/R578X, R613X/R1683X and normal human dermal fibroblasts were plated at a density of 300,000 cells per well of a 6-well plates. On the following day, cells were untreated or treated with test compounds for 24 hours (tested range: 10-60 μM, see table 4 for details). Media and compound were then replaced with fresh media containing indicated doses of compounds. After 48 hours of treatment, cells were sub-cultured onto coverslips and subjected to a well-established fibroblast migration assay as follows: Colloidal gold salts were immobilized on coverslips and covered with type I collagen (15 mg/ml). Fibroblast cultures were suspended, plated on the coverslips, and allowed to migrate for 16-20 hours. The cells were fixed in 0.1% formaldehyde in phosphate-buffered saline and examined under dark field optics. 15-20 non-overlapping fields in each experimental condition were analyzed with NIH Image 1.6 and the percentage area of each field consumed by cell migration tracks was determined (termed Migration Index, MI). In this assay migration indexes of treated and untreated RDEB cells were compared to normal human fibroblast (NHF) cells as well as RDEB cells treated with gentamicin (previously shown to rescue hypermotility phenotype by Cogan et al., 2014; positive control).

The ability of the test compounds in inducing the expression of full-length Collagen VII protein in two RDEB patient fibroblast cell lines with R578X/R578X and R613X/R1683X mutations was assessed after 48 hours of treatment. Compounds were ranked by considering the MI of compound treated fibroblasts relative to untreated and NHF cells. In this assay untreated cells are most motile, (with little/no readthrough; marked as +) while NHFs are least motile. Compounds that induce a high level of readthrough have a similar motility level (and therefore MI) to NHFs (marked as +++).

| | Full rescue | Readthrough Activity as Measured |
|---|---|---|
| Cmpd. | at tested | by Fibroblast Hypermotility |
| No. | Conc. (μM) | Phenotype Rescue |
| 198 | 20 | +++ |
| 60 | 50 | +++ |
| 179 | 25 | +++ |
| 241 | 50 | +++ |

Example 3: CF Assay Rational, Methodology and Results

### Automated Equivalent Current (IEQ) Assay

### Rationale

The efficacy of compounds in increasing chloride ion transport by inducing readthrough of Cystic Fibrosis Transmembrane Conductance Regulator (CFTR) nonsense mutation was assessed in primary CF human Bronchial Epithelial (hBE) cultures after 96 hours of treatment using a TECC24 assay setup.

### Methodology

This method has previously been described in detail (Vu et al., Journal of medicinal chemistry vol. 60,1 (2017): 458-473). Cell culture: primary CF hBE cells from a donor with the G542X/F508del-CFTR mutation (patient code KK34J) were cultured as monolayers on Transwell 24-well filter plates at an Air Liquid Interface (ALI) for 4-6 weeks until fully differentiated. Treatment procedure: cultures were basolaterally treated with media containing test compounds (diluted in DMSO) at a final concentration of 20 μM and 60 μM. After 2 days of incubation, basolateral media was replaced with fresh media containing test compounds and the apical mucus was washed with media for at least 30 minutes and incubated for a further 2 days (total of 96 hours). 125 μg/mL ELX-02 was used as a positive control and a CFTR activity standard for readthrough ranking. I_{EQ} measurements: custom software was used to measure transepithelial voltage (V_{T}) and conductance (G_{T}) with a 24-channel current clamp circuit and electrode manifold (TECC24; EP Design BVBA, Bertem, Belgium) coupled to a cartesian robot. Measurements were made every ˜5 minutes. Electrodes were washed between each plate read cycle. Baseline reads were measured for ˜20 minutes. After this period for each epithelium, 25 μL of 100 μM benzamil (10 μM final concentration) was added apically to block ENaC currents, and readings were taken for an additional 20 minutes. After this period, forskolin (10 μM final concentration) and the potentiator VX-770 (100 nM final concentration) were added apically (27.8 μL of combined 100 μM forskolin, 1 μM VX-770 stock). After an additional ˜20 minutes, 30.9 μL of 200 μM CFTRinh-172 (20 μM final concentration) was added to the apical solution to terminate the run by inhibiting the CFTR-dependent current. Data analysis: The Ohm's law equation I_{EQ}=V_{T}G_{T} was used to calculate current using Microsoft Excel software. I_{EQ} responses to the above experimental reagents were calculated at each step. The change in current from the baseline that is resultant from the simultaneous addition of forskolin and VX-770 is termed activation current and was used to determine the level of CFTR activity (and hence readthrough) in these studies.

Activity data is expressed as the percent activation relative to vehicle treated (00%) and ELX-02 (100%) treated cells: ++≤10%, ++=11-50%, +++=51-100%, ++++=>100%.

| Compound No. | Activation/Readthrough Level |
|---|---|
| 5 | + |
| 6 | + |
| 38 | + |
| 48 | +++ |
| 55 | ++ |
| 60 | ++ |
| 84 | ++ |
| 89 | ++ |
| 91 | +++ |
| 95 | + |
| 97 | ++ |
| 99 | + |
| 112 | + |
| 132 | +++ |
| 134 | ++ |
| 151 | ++ |
| 158 | +++ |
| 195 | + |
| 198 | ++ |
| 204 | + |
| 210 | +++ |
| 215 | +++ |
| 220 | ++ |
| 225 | ++ |
| 230 | + |
| 241 | +++ |
| 242 | ++ |
| 246 | +++ |
| 247 | + |
| 248 | +++ |
| 260 | + |

Example 4: In Vivo APCmin Study

ApcMin mice (C57BL/6J-ApcMin/J)JAX, Jackson laboratory, Bar Harbor ME) harboring the L850X mutation are randomized and dosed with either test agent or vehicle control starting at 10 weeks of age. The mice are dosed daily for a period of 8 weeks at which time the mice are sacrificed, and small intestine, large intestine and spleen are harvested. Spleen weights are measured and recorded. Photographs of the whole small intestine and partial large intestine (˜300-400 mm) are captured. The middle section of each small intestine is fixed in 10% neutral buffered formalin (NBF, RT) for 24-36 h and transferred to ethanol. Subsequently, all samples are processed to FFPE blocks for histology analysis.

During the dosing phase of the study animal body weights are measured and recorded daily. During the dosing phase, any mice showing anemia, >20% body weight loss or clinical signs of pain are euthanized, and small intestine, large intestine and spleen are harvested. Efficacy of test agent is assessed by comparing Polyp counts, spleen weights and histopathology assessments between the vehicle treat mice and the test agent treated mice.

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set in verbatim herein herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method for treating a subject having a genetic disease comprising:
administering a therapeutically effective amount of a compound of Formula I: 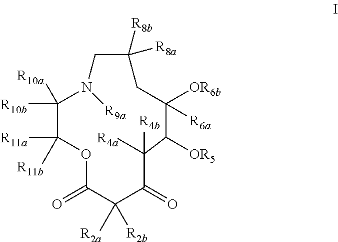
or a pharmaceutically acceptable salt thereof, wherein:
one of R₂ₐ and R_{2b} is selected from the group consisting of H, halo, optionally substituted C₁₋₁₀ alkyl, optionally substituted C₁₋₁₀ alkoxy, and optionally substituted C₂₋₁₀ alkenyl, wherein C₁₋₁₀ alkyl, C₁₋₁₀ alkoxy, and C₂₋₁₀ alkenyl are optionally substituted with one or more groups selected from the group consisting of halo, aryl, amino, alkyl, heteroalkyl, heteroalkenyl, heterocycloalkyl, and heteroaryl; and the other of R₂ₐ and R_{2b} is selected from the group consisting of halo, optionally substituted C₁₋₁₀ alkyl, optionally substituted C₁₋₁₀ alkoxy, and optionally substituted C₂₋₁₀ alkenyl, wherein C₁₋₁₀ alkyl, C₁₋₁₀ alkoxy, and C₂₋₁₀ alkenyl are optionally substituted with one or more groups selected from the group consisting of halo, aryl, amino, alkyl, heteroalkyl, heteroalkenyl, heterocycloalkyl, and heteroaryl;
each of R₄ₐ and R_{4b} is independently selected from the group consisting of H and optionally substituted C₁₋₁₀ alkyl;
R₅ is selected from the group consisting of H, a hydroxyl protecting group, and 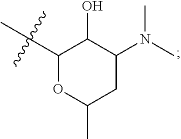 [SMILES: *C1OC(C)CC(N(C)C)C1O]
R₆ₐ is optionally substituted C₁₋₁₀ alkyl;
R_{6b} is H, C₁₋₁₀ alkyl, C₁₋₁₀ hydroxyalkyl, allyl, haloalkyl, aryl, heteroalkenyl, heterocycloalkyl, or heteroaryl, any of which can be optionally substituted with one or more groups selected from the group consisting of halo, aryl, amino, heteroalkyl, heteroalkenyl, heterocycloalkyl, and heteroaryl;
R₈ₐ and R_{8b} are each independently selected from the group consisting of H and optionally substituted C₁₋₁₀ alkyl;
R₉ₐ is selected from the group consisting of H, optionally substituted C₁₋₁₀ alkyl, C(═O)C₁₋₆ alkyl, C₁₋₆ alkylene-OH, C₁₋₆ alkylene-O—C₁₋₆ alkyl, C₁₋₆ alkylene-cyclolkyl, C(═O)C₁₋₆ alkylene-cycloalkyl, C(═O)cycloalkyl, C(═O)heterocycloalkyl, C(═O)aryl, C(═O)heteroaryl or C(═O)NH-aryl;
one of R₁₀ₐ and R_{10b} is selected from the group consisting of H and optionally substituted C₁₋₁₀ alkyl, and the other of R₁₀ₐ and R_{10b} is
Lₐ-L_{b}-L_{c}-L_{d} wherein:
Lₐ is C₂₋₆ alkyenylene or C₁₋₆ alkylene, wherein one methylene unit of C₁₋₆ alkylene may be replaced by oxo 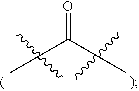 [SMILES: *C(*)=O]
L_{b} is absent, or is optionally substituted cycloalkyl or optionally substituted heterocycloalkyl;
L_{c} is absent or is C₁₋₆ alkylene, C₁₋₆ alkylene-N(C₁₋₆ alkyl), NH, N—C₁₋₆ alkyl, N—C₁₋₆ cycloalkyl, —NH—C₁₋₆ alkylene, —N(C₁₋₆ alkyl)-C₁₋₆ alkylene, —NH—C₁₋₆ alkylene-heteroarylene, —N—C₁₋₆ alkylene-cycloalkyl, N—C₁₋₆ alkylene-heterocycloalkyl, N—C₁₋₆ alkylene-aryl, N—C₁₋₆ alkylene-heteroaryl, C(═O), C(═O)O—, OC(O)—, C(═O)NH, C(═O)N-alkyl, C(═O)NH—C₁₋₆ alkylene, C(═O)N(C₁₋₆ alkyl)-C₁₋₆ alkylene, N(C₁₋₆ alkyl)-C(═O)—C₁₋₆ alkylene, SO₂, SO₂NH, SO₂N—C₁₋₆ alkyl, SO₂N—(C₁₋₆ alkyl)-(C₁₋₆ alkylene), OC(═O)—NH, OC(═O)—N-alkyl, SO₂, SO₂C₁₋₆ alkylene, SO₂NH, SO₂N(C₁₋₆ alkyl), SO₂NH—(C₁₋₆ alkylene), SO₂N(C₁₋₆ alkyl)-(C₁₋₆ alkylene), SO₂N(C₁₋₆ alkylene-aryl), SO₂N(C₁₋₆ alkylene-heteroaryl), 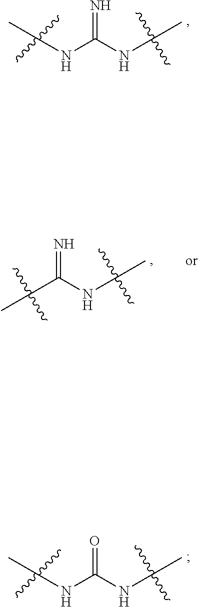 [SMILES: *NC(=N)N* ; *NC(*)=N ; *NC(=O)N*]
L_{d} is H, C₁₋₆ alkyl, OH, alkoxy, NH₂, NHC₁₋₆ alkyl, N(C₁₋₆ alkyl)₂, C₁₋₆ alkyl, C₁₋₆ haloalkyl, optionally substituted cycloalkyl, optionally substituted heterocycloalkyl optionally substituted aryl, optionally substituted heteroaryl, optionally substituted benzyl; and
R₁₁ₐ and R_{11b} are each independently selected from the group consisting of —H and optionally substituted C₁₋₁₀ alkyl;
wherein “ [symbol] ” indicates a point of attachment.

2. The method of claim 1, wherein the compound of formula I is a compound of formula IH 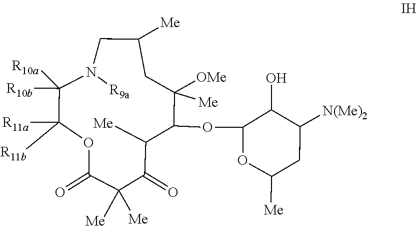
or a pharmaceutically acceptable salt thereof.

3. The method of claim 2, wherein R_{10b}, R₁₁ₐ, and R_{11b} are each independently H or methyl.

4. The method of claim 2, wherein R₉ₐ is —H, methyl, ethyl, propyl, isopropyl, butyl, isobutyl, pentyl, isopentyl, acetyl, C(═O)—NH-phenyl, C(═O)-ethyl, C(═O)-cyclopropyl, CH₂-cyclobutyl, CH₂CH₂CH₂OH, CH₂CH₂CH₂OMe, or CH₂CH₂OH,

5. The compound of claim 2, wherein Lₐ of -Lₐ-L_{b}-L_{c}-L_{d} is C₂₋₆ alkenylene; L_{b} and L_{c} are absent, and L_{d} is H.

6. The method of claim 5, wherein Lₐ is —CH₂═CH₂—, L_{b} and L_{c} are absent, and L_{d} is H.

7. The method of claim 2, wherein Lₐ is C₁₋₆ alkylene selected from the group consisting of —CH₂—, —CH₂CH₂—, —CH₂CH₂CH₂—, and —CH₂CH₂CH₂CH₂—, wherein one methylene unit of —CH₂—, —CH₂CH₂—, —CH₂CH₂CH₂—, or —CH₂CH₂CH₂CH₂— can optionally be replaced by oxo (C═O).

8. The method of claim 7, wherein Lₐ is CH₂ or oxo (C═O); L_{b} is absent; L_{c} is CO, C(═O)NH—C₁₋₆ alkylene, —NH—C₁₋₆ alkylene, or N(C₁₋₆ alkyl)-C₁₋₆ alkylene; and L_{d} is H, optionally substituted cycloalkyl, optionally substituted heterocycloalkyl optionally substituted aryl, optionally substituted heteroaryl, or N(C₁₋₆ alkyl)₂.

9. The method of claim 2, wherein:
Lₐ is CH₂ or oxo;
L_{b} is optionally substituted cycloalkyl or heterocycloalkyl selected from the group consisting of cyclobutyl, cyclopentyl, cyclohexyl, azetidinyl, pyrrolidinyl, piperidinyl, piperazinyl, and morpholinyl, any of which may be optionally substituted;
L_{c} is absent or is C₁₋₆ alkylene, C₁₋₆ alkylene-N(C₁₋₆ alkyl), NH, N—C₁₋₆ alkyl, N—C₁₋₆ cycloalkyl, —NH—C₁₋₆ alkylene, —N(C₁₋₆ alkyl)-C₁₋₆ alkylene, —N—C₁₋₆ alkylene-cycloalkyl, N—C₁₋₆ alkylene-heterocycloalkyl, N—C₁₋₆ alkylene-aryl, N—C₁₋₆ alkylene-heteroaryl, C(═O), C(═O)O—, OC(O)—, C(═O)NH, C(═O)N-alkyl, C(═O)NH—C₁₋₆ alkylene, C(═O)N(C₁₋₆ alkyl)-C₁₋₆ alkylene, SO₂, SO₂NH, SO₂N—C₁₋₆ alkyl, OC(═O)—NH, OC(═O)—N-alkyl, N(C₁₋₆ alkyl)-C(═O)—C₁₋₆ alkylene, SO₂, SO₂C₁₋₆ alkylene, SO₂NH, SO₂N(C₁₋₆ alkyl), SO₂NH—(C₁₋₆ alkylene), SO₂N(C₁₋₆ alkyl)-(C₁₋₆ alkylene), SO₂N(C₁₋₆ alkylene-aryl), SO₂N(C₁₋₆ alkylene-heteroaryl), 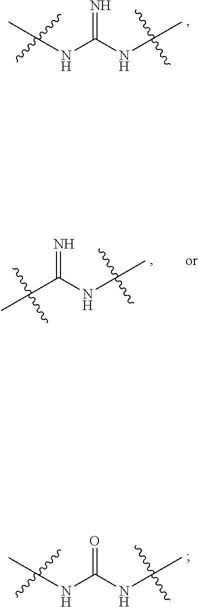 [SMILES: *NC(=N)N* ; *NC(*)=N ; *NC(=O)N*]
and
L_{d} is H, —OH, C₁₋₆ alkyl, C₁₋₆ haloalkyl, optionally substituted cycloalkyl, optionally substituted heterocycloalkyl optionally substituted aryl, or optionally substituted heteroaryl.

10. The method of claim 9, wherein:
Lₐ is CH₂ or C(═O);
L_{b} is azetidinyl, piperidinyl, piperazinyl, pyrrolidinyl, or morpholinyl;
L_{c} is absent or is C₁₋₆ alkylene, C(═O), C(═O)O—, C(═O)NH, C(═O)N-alkyl, C(═O)NH—C₁₋₆ alkylene, C(═O)N(C₁₋₆ alkyl)-C₁₋₆ alkylene; and
L_{d} is H, —OH, C₁₋₆ alkyl, C₁₋₆ haloalkyl, optionally substituted cycloalkyl, optionally substituted heterocycloalkyl, optionally substituted aryl, or optionally substituted heteroaryl.

11. The method of claim 9, wherein:
Lₐ is CH₂ or oxo;
L_{b} is piperidinyl, piperazinyl, pyrrolidinyl, or morpholinyl;
L_{c} is C(═O), C(═O)O—, C(═O)NH, C(═O)N-alkyl, C(═O)NH—C₁₋₆ alkylene, C(═O)N(C₁₋₆ alkyl)-C₁₋₆ alkylene; and
L_{d} is H, methyl, ethyl, isopropyl, isobutyl, t-butyl, triofluoromethyl, hydroxy, methoxy, phenyl, 2-methoxyphenyl, 3-methoxyphenyl, 4-methoxyphenyl, 2-chlorophenyl, 3-chlorophenyl, 4-chlorophenyl, 2-toluyl, 3-toluyl, 4-toluyl, 2-trifluoromethylphenyl, 3-trifluoromethylphenyl, 4-trifluoromethylphenyl, 3-trifluoromethylphenyl, 4-trifluoromethylphenyl, naphthyl, imidazolyl, 2-pyridyl, 3-pyridyl, 4-pyridyl,

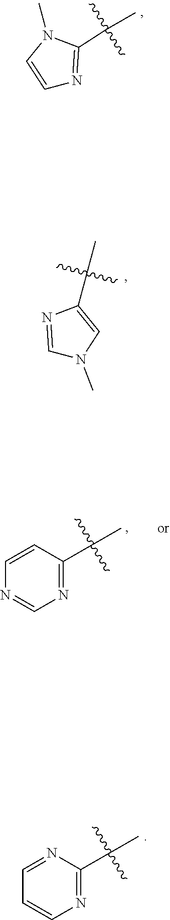

12. The method of claim 9, wherein:
LₐL_{b} is 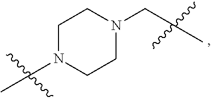 [SMILES: *CN1CCN(*)CC1]
L_{c} is absent, and L_{d} is H;
LₐL_{b} is 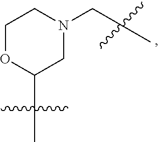 [SMILES: *CN1CCOC(*)C1]
L_{c} is absent, and L_{d} is H; or
LₐL_{b} is 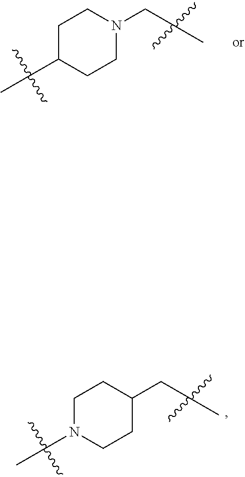 [SMILES: *CN1CCC(*)CC1 ; *CC1CCN(*)CC1]
L_{c} is absent, and L_{d} is H.

13. The method of claim 9, wherein:
LₐL_{b} is 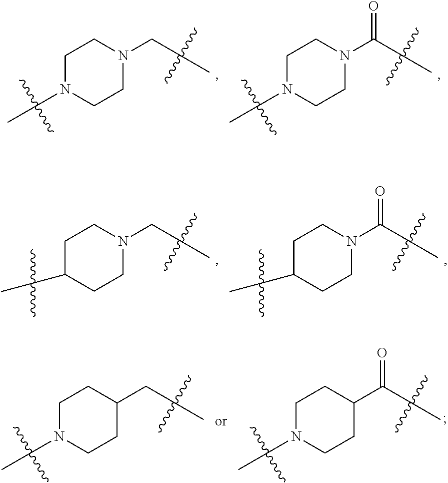 [SMILES: *CN1CCN(*)CC1 ; *C(=O)N1CCN(*)CC1 ; *CN1CCC(*)CC1 ; *C(=O)N1CCC(*)CC1 ; *CC1CCN(*)CC1 ; *C(=O)C1CCN(*)CC1]
L_{c} is absent or is CH₂, CH₂CH₂, CHCH₃, CH₂CHCH₃, C(CH₃)₂; and
L_{d} is H, —OH, —OMe, methyl, ethyl, isopropyl, isobutyl, tert-butyl, trifluoromethyl, cyclopropyl, cyclobutyl, phenyl, trifluormethyl, 2-methoxyphenyl, 3-methoxyphenyl, 4-methoxyphenyl, 2-chlorophenyl, 3-chlorophenyl, 4-chlorophenyl, 2-toluyl, 3-toluyl, 4-toluyl, 2-trifluoromethylphenyl, 3-trifluoromethylphenyl, 4-trifluoromethylphenyl, 3-trifluoromethylphenyl, 4-trifluoromethylphenyl, naphthyl, imidazolyl, 2-pyridyl, 3-pyridyl, 4-pyridyl,

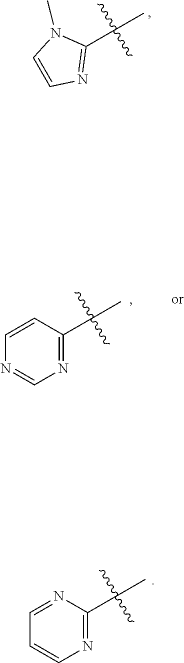

14. The method of claim 9, wherein:
LₐL_{b} is 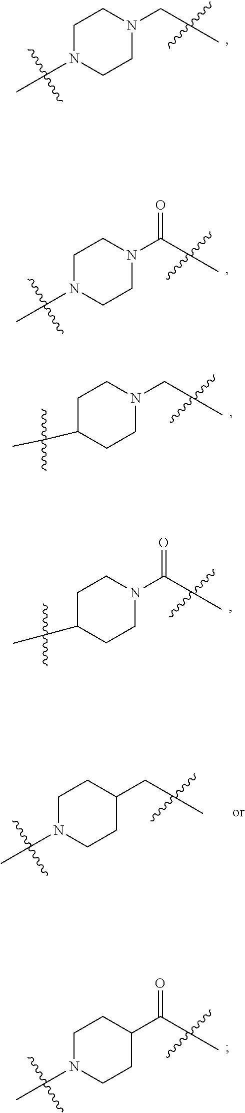 [SMILES: *CN1CCN(*)CC1 ; *C(=O)N1CCN(*)CC1 ; *CN1CCC(*)CC1 ; *C(=O)N1CCC(*)CC1 ; *CC1CCN(*)CC1 ; *C(=O)C1CCN(*)CC1]
L_{c} is oxo; and
L_{d} is H, methyl,ethyl, isopropyl, isobutyl, tert-butyl, cyclopropyl, phenyl, trifluormethyl, 2-methoxyphenyl, 3-methoxyphenyl, 4-methoxyphenyl, 2-chlorophenyl, 3-chlorophenyl, 4-chlorophenyl, 2-toluyl, 3-toluyl, 4-toluyl, 2-trifluoromethylphenyl, 3-trifluoromethylphenyl, 4-trifluoromethylphenyl, 3-trifluoromethylphenyl, 4-trifluoromethylphenyl, naphthyl, imidazolyl, 2-pyridyl, 3-pyridyl, 4-pyridyl,

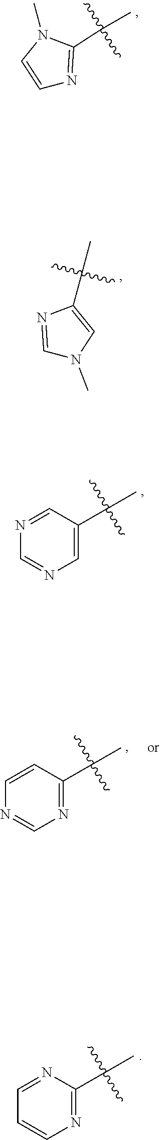

15. The method of claim 9, wherein:
LₐL_{b} is 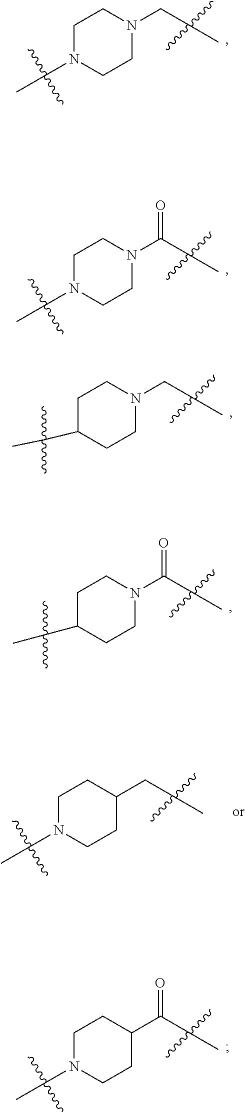 [SMILES: *CN1CCN(*)CC1 ; *C(=O)N1CCN(*)CC1 ; *CN1CCC(*)CC1 ; *C(=O)N1CCC(*)CC1 ; *CC1CCN(*)CC1 ; *C(=O)C1CCN(*)CC1]
L_{c} is SO₂, SO₂C₁₋₆ alkylene, SO₂NH, SO₂N—C₁₋₆ alkyl, SO₂N—(C₁₋₆ alkyl)(C₁₋₆ alkylene); and
L_{d} is H, methyl, ethyl, propyl, isopropyl, phenyl, 2-methoxyphenyl, 3-methoxyphenyl, 4-methoxyphenyl, 2-chlorophenyl, 3-chlorophenyl, 4-chlorophenyl, 2-toluyl, 3-toluyl, 4-toluyl, 2-trifluoromethylphenyl, 3-trifluoromethylphenyl, 4-trifluoromethylphenyl, 3-trifluoromethylphenyl, 4-trifluoromethylphenyl, naphthyl,

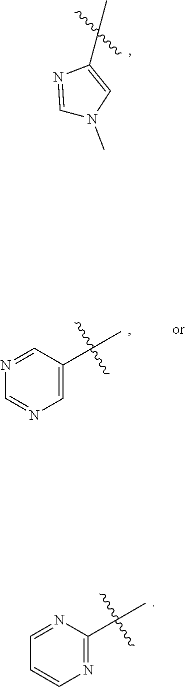

16. The method of claim 9, wherein:
LₐL_{b} is 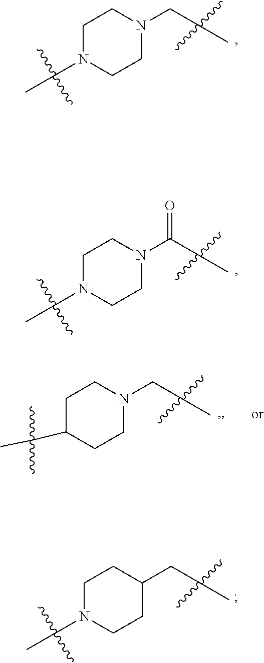 [SMILES: *CN1CCN(*)CC1 ; *C(=O)N1CCN(*)CC1 ; *CN1CCC(*)CC1 ; *CC1CCN(*)CC1]
L_{c} is absent or is C₁₋₆ alkylene, C(═O), C(═O)O, C(═O)NH, C(═O)N-alkyl, C(═O)NH—C₁₋₆ alkylene, C(═O)N(C₁₋₆ alkyl)-C₁₋₆ alkylene; and
L_{d} is H, hydroxyl, C₁₋₆ alkyl, alkoxy, C₁₋₆ haloalkyl, optionally substituted cycloalkyl, optionally substituted heterocycloalkyl optionally substituted aryl, optionally substituted heteroaryl.

17. The method of claim 9, wherein:
Lₐ is CH₂ or oxo;
L_{b} is cyclobutyl;
L_{c} is absent or is —NH—C₁₋₆ alkylene, or N(C₁₋₆ alkyl)-C₁₋₆ alkylene; and
L_{d} is H, methyl, ethyl, isopropyl, cyclopropyl, imidazolyl, 2-pyridyl, 3-pyridyl, 4-pyridyl,

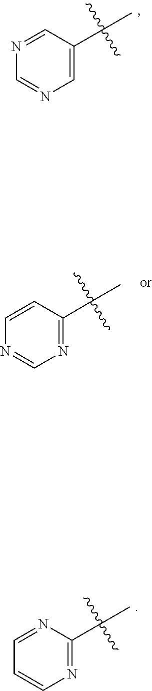 [SMILES: *c1cncnc1 ; *c1ccncn1 ; *c1ncccn1]

18. The compound of claim 9, wherein:
LₐL_{b} is 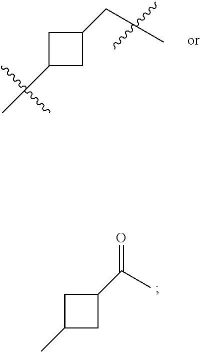
L_{c} is NHCH₂—, N(Me)CH₂—, N(Et)CH₂—, N(iPr)CH₂—, N(isobutyl)CH₂—,; and
L_{d} is H or cyclopropyl.

19. The method of claim 1 using a compound of formula A: 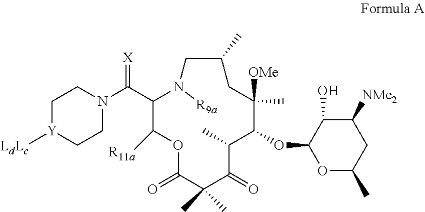
or a pharmaceutically acceptable salt thereof, wherein:
X is H₂ or O;
Y is N, CH, C—(C₁₋₆ alkyl), or O;
R₉ₐ is selected from the group consisting of H, optionally substituted C₁₋₆ alkyl, C₁₋₆ alkylene-OH, C₁₋₆ alkylene-O—C₁₋₆ alkyl, C(═O)C₁₋₆ alkyl, C₁₋₆ alkylene-cyclolkyl, or C(═O)cycloalkyl;
L_{c} is absent or is C₁₋₄ alkylene, C₁₋₄ alkylene-N(C₁₋₄ alkyl), NH, N—C₁₋₄ alkyl, N—C₁₋₄ cycloalkyl, —NH—C₁₋₄ alkylene, —N(C₁₋₄ alkyl)-C₁₋₄ alkylene, —N—C₁₋₄ alkylene-cycloalkyl, N—C₁₋₄ alkylene-heterocycloalkyl, N—C₁₋₄ alkylene-aryl, N—C₁₋₄ alkylene-heteroaryl, C(═O), C(═O)O—, C(═O)NH, C(═O)N-alkyl, C(═O)NH—C₁₋₄ alkylene, C(═O)N(C₁₋₄ alkyl)-C₁₋₄ alkylene SO₂, SO₂NH, SO₂N—C₁₋₄ alkyl, SO₂N—(C₁₋₄ alkyl)-(C₁₋₄ alkylene), OC(═O)—NH, OC(═O)—N—C₁₋₄ alkyl, SO₂, SO₂C₁₋₄ alkylene, SO₂NH, SO₂N(C₁₋₄ alkyl), SO₂NH—(C₁₋₄ alkylene, SO₂N(C₁₋₄ alkyl)-(C₁₋₄ alkylene), SO₂N(C₁₋₄ alkylene-aryl), SO₂N(C₁₋₄ alkylene-heteroaryl), 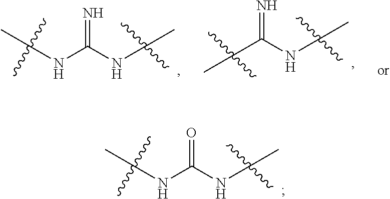 [SMILES: *NC(=N)N* ; *NC(*)=N ; *NC(=O)N*]
L_{d} is H, C₁₋₆ alkyl, OH, alkoxy, NH₂, NHC₁₋₆ alkyl, N(C₁₋₆ alkyl)₂, C₁₋₆ haloalkyl, optionally substituted cycloalkyl, optionally substituted heterocycloalkyl optionally substituted aryl, optionally substituted heteroaryl, optionally substituted benzyl; and
R₁₁ₐ is H or C₁₋₆ alkyl.

20. The method of claim 19, wherein:
R₉ₐ is —H, methyl, ethyl, propyl, isopropyl, butyl, isobutyl, acetyl, C(═O)-ethyl, C(═O)-cyclopropyl, CH₂-cyclobutyl, CH₂CH₂CH₂OH, CH₂CH₂CH₂OMe, or CH₂CH₂OH;
R₁₁ₐ is H or methyl;
L_{c} is absent or is C₁₋₄ alkylene, NH, N(C₁₋₄ alkyl), N(C₁₋₄ alkyl)-C₁₋₄ alkylene, C(═O), C(═O)O—, C(═O)NH, C(═O)N-alkyl, C(═O)NH—C₁₋₄ alkylene, OC(═O)—NH, SO₂, SO₂C₁₋₄ alkylene, SO₂NH, SO₂N(C₁₋₄ alkyl), or 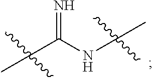 [SMILES: *NC(*)=N]
L_{d} is H, NH₂, NH(C₁₋₄ alkyl), N(C₁₋₄ alkyl)₂, trifluoromethyl, methyl, ethyl, isopropyl, isobutyl, t-butyl, hydroxy, methoxy, cyclopropyl, cyclobutyl, phenyl, 2-methoxyphenyl, 3-methoxyphenyl, 4-methoxyphenyl, 2-chlorophenyl, 3-chlorophenyl, 4-chlorophenyl, 2-toluyl, 3-toluyl, 4-toluyl, 2-trifluoromethylphenyl, 3-trifluoromethylphenyl, 4-trifluoromethylphenyl, 3-trifluoromethylphenyl 4-trifluoromethylphenyl, naphthyl, imidazolyl, 2-pyridyl, 3-pyridyl, 4-pyridyl,

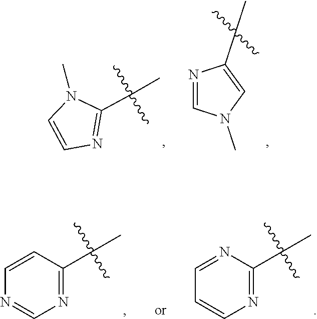

21. The method of claim 19, wherein:
X is H₂ or O;
Y is Nor CH,; and
R₁₁ₐ is H or methyl.

22. The method of claim 1 using a compound of formula A1: 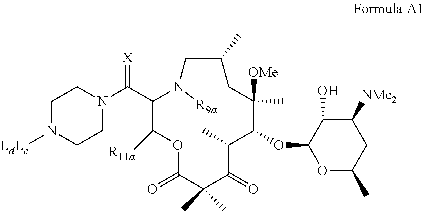
or a pharmaceutically acceptable salt thereof, wherein:
X is H₂ or O;
R₉ₐ is H or C₁₋₄alkyl;
R₁₁ₐ is H or C₁₋₄alkyl;
L_{c} is absent or is C₁₋₃ alkylene, C(═O), C(═O)NH, C(═O)NH—C₁₋₃ alkylene, or SO₂C₁₋₃alkylene; and
L_{d} is NH(C₁₋₄ alkyl), N(C₁₋₄ alkyl)₂, C₃₋₆ cycloalkyl, 6-10 membered aryl, or 5-10 membered heteroaryl, wherein the cycloalkyl, aryl, and heteroaryl are each independently and optionally substituted with halo, C₁₋₄alkyl, C₁₋₄haloalkyl, or C₁₋₄alkoxy.

23. The method of claim 22, wherein:
R₉ₐ is H, Me, Et, Pr, butyl, or isopropyl;
R₁₁ₐ is H or Me;
L_{c} is CH₂, CH₂CH₂, C(═O)NH, C(═O), C(═O)NHCH₂, or SO₂CH₂; and
L_{d} is N(CH₃)₂, NHCH(CH₃)₂, cyclopropyl, cyclobutyl, phenyl, naphthalenyl, imidazolyl, pyrimidinyl, or pyridinyl, wherein phenyl, imidazolyl, pyrimidinyl, and pyridinyl are each independently and optionally substituted with F, Cl, Br, Me, OMe, or CF₃.

24. The method of claim 1 using a compound of formula A2: 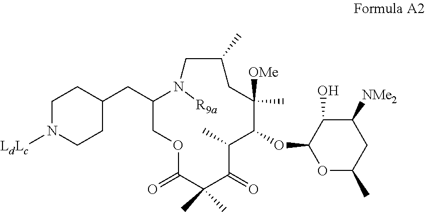
or a pharmaceutically acceptable salt thereof, wherein:
R₉ₐ is H or C₁₋₄alkyl;
L_{c} is C₁₋₃ alkylene, C(═O), C(═O)NH, C(═O)NH—C₁₋₃ alkylene, SO₂, or SO₂C₁₋₃alkylene; and
L_{d} is OH, NH(C₁₋₄ alkyl), N(C₁₋₄ alkyl)₂, 6-10 membered aryl or 5-10 membered heteroaryl, wherein the aryl and heteroaryl are each independently and optionally substituted with halo, C₁₋₄alkyl, C₁₋₄haloalkyl, or C₁₋₄alkoxy.

25. The method of claim 24, wherein
R₉ₐ is H, Me, Et, Pr, butyl, or isopropyl;
L_{c} is CH₂, CH₂CH₂, C(═O), C(═O)NH, C(═O)NHCH₂, SO₂, or SO₂CH₂; and
L_{d} is OH, N(CH₃)₂, NHCH(CH₃)₂, phenyl, or pyridinyl, wherein phenyl and pyridinyl are each independently and optionally substituted with F, Cl, Br, Me, OMe, or CF₃.

26. The method of claim 1 using a compound of formula B: 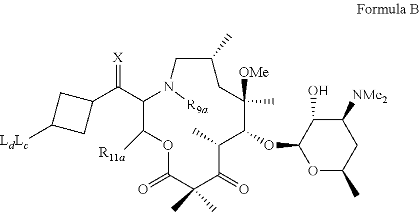
or a pharmaceutically acceptable salt thereof, wherein:
X is H₂ or O;
R₉ₐ is selected from the group consisting of H, optionally substituted C₁₋₆ alkyl, C₁₋₆ alkylene-OH, C₁₋₆ alkylene-O—C₁₋₆ alkyl, C(═O)C₁₋₆ alkyl, C₁₋₆ alkylene-cyclolkyl, C(═O)cycloalkyl, or C(═O)NH-aryl;
L_{c} is absent or is C₁₋₄ alkylene, C₁₋₄ alkylene-N(C₁₋₄ alkyl), NH, N—C₁₋₄ alkyl, N—C₁₋₄ cycloalkyl, —NH—C₁₋₄ alkylene, —N(C₁₋₄ alkyl)-C₁₋₄ alkylene, —N—C₁₋₄ alkylene-cycloalkyl, N—C₁₋₄ alkylene-heterocycloalkyl, N—C₁₋₄ alkylene-aryl, N—C₁₋₄ alkylene-heteroaryl, C(═O), C(═O)O—C(═O)NH, C(═O)N-alkyl, C(═O)NH—C₁₋₄ alkylene, C(═O)N(C₁₋₄ alkyl)-C₁₋₄ alkylene SO₂, SO₂NH, SO₂N—C₁₋₄ alkyl, SO₂N—(C₁₋₄ alkyl)-(C₁₋₄ alkylene), OC(═O)—NH, OC(═O)—N—C₁₋₄ alkyl, SO₂, SO₂C₁₋₄ alkylene, SO₂NH, SO₂N(C₁₋₄ alkyl), SO₂NH—(C₁₋₄ alkylene, SO₂N(C₁₋₄ alkyl)-(C₁₋₄ alkylene), SO₂N(C₁₋₄ alkylene-aryl), SO₂N(C₁₋₄ alkylene-heteroaryl), 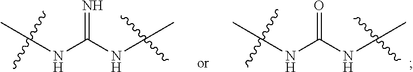 [SMILES: *NC(=N)N* ; *NC(=O)N*]
L_{d} is H, C₁₋₆ alkyl, OH, alkoxy, NH₂, NHC₁₋₆ alkyl, N(C₁₋₆ alkyl)₂, C₁₋₆ haloalkyl, optionally substituted cycloalkyl, optionally substituted heterocycloalkyl optionally substituted aryl, optionally substituted heteroaryl, optionally substituted benzyl; and
R₁₁ₐ is H or C₁₋₆ alkyl.

27. The method of claim 26, wherein:
R₉ₐ is —H, methyl, ethyl, propyl, isopropyl, butyl, isobutyl, acetyl, C(═O)-ethyl, C(═O)-cyclopropyl, CH₂-cyclobutyl, CH₂CH₂CH₂OH, CH₂CH₂CH₂OMe, or CH₂CH₂OH;
R₁₁ₐ is H or methyl;
L_{c} is absent or is C₁₋₄ alkylene, NH, N—C₁₋₄ alkyl, or —N(C₁₋₄ alkyl)-C₁₋₄ alkylene; and
L_{d} is H, C₁₋₆ alkyl, or C₁₋₄ cycloalkyl.

28. The method of claim 26, wherein:
X is H₂ or O;
R₉ₐ is —H or methyl; and
R₁₁ₐ is H or methyl.

29. The method of claim 9, wherein Lₐ is CH₂CH₂ or CH₂CH₂CH₂, wherein one methylene unit of Lₐ can optionally be replaced by oxo.

30. The method of claim 29, wherein:
Lₐ is CH₂CH₂, CH₂CH₂CH₂, COCH₂, COCH₂CH₂, CH₂COCH₂;
L_{b} is absent, or is optionally substituted cycloalkyl or optionally substituted heterocycloalkyl;
L_{c} is absent or is C₁₋₆ alkylene, C₁₋₆ alkylene-N(C₁₋₆ alkyl), NH, N—C₁₋₆ alkyl, N—C₁₋₆ cycloalkyl, —NH—C₁₋₆ alkylene, —NH—C₁₋₆ alkylene-heteroarylene, —N(C₁₋₆ alkyl)-C₁₋₆ alkylene, —N—C₁₋₆ alkylene-cycloalkyl, N—C₁₋₆ alkylene-heterocycloalkyl, N—C₁₋₆ alkylene-aryl, N—C₁₋₆ alkylene-heteroaryl, C(═O), C(═O)O—, OC(O)—, C(═O)NH, C(═O)N-alkyl, C(═O)NH—C₁₋₆ alkylene, C(═O)N(C₁₋₆ alkyl)-C₁₋₆ alkylene SO₂, SO₂ C₁₋₆ alkylene, SO₂NH, SO₂N—C₁₋₆ alkyl, OC(═O)—NH, OC(═O)—N-alkyl, SO₂, SO₂C₁₋₆ alkylene, SO₂NH, SO₂N(C₁₋₆ alkyl), SO₂NH—(C₁₋₆ alkylene, SO₂N(C₁₋₆ alkyl)-(C₁₋₆ alkylene), SO₂N(C₁₋₆ alkylene-aryl), SO₂N(C₁₋₆ alkylene-heteroaryl), 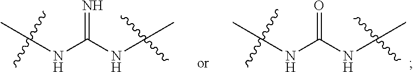 [SMILES: *NC(=N)N* ; *NC(=O)N*]
and
L_{d} is H, C₁₋₆ alkyl, OH, alkoxy, NH₂, NHC₁₋₆ alkyl, N(C₁₋₆ alkyl)₂, C₁₋₆ alkyl, C₁₋₆ haloalkyl, optionally substituted cycloalkyl, optionally substituted heterocycloalkyl, optionally substituted aryl, or optionally substituted heteroaryl.

31. The method of claim 29, wherein:
Lₐ is CH₂CH₂, CH₂CH₂CH₂, COCH₂, COCH₂CH₂, CH₂COCH₂;
L_{b} is C₃₋₆cycloalkyl or 5-6 membered heterocycloalkyl;
L_{c} is absent; and
L_{d} is H, OH, C₁₋₆ alkyl, OH, alkoxy, NH₂, NHC₁₋₆ alkyl, N(C₁₋₆ alkyl)₂, C₁₋₆ alkyl, C₁₋₆ haloalkyl, optionally substituted C₃₋₆cycloalkyl, optionally substituted 3-6 membered heterocycloalkyl, optionally substituted phenyl, or optionally substituted 5-10 membered heteroaryl.

32. The method of claim 29, wherein:
Lₐ is CH₂CH₂, CH₂CH₂CH₂, COCH₂, COCH₂CH₂, CH₂COCH₂;
L_{b} is absent;
L_{c} is absent; and
L_{d} is pyrrolidinyl, oxazolyl, 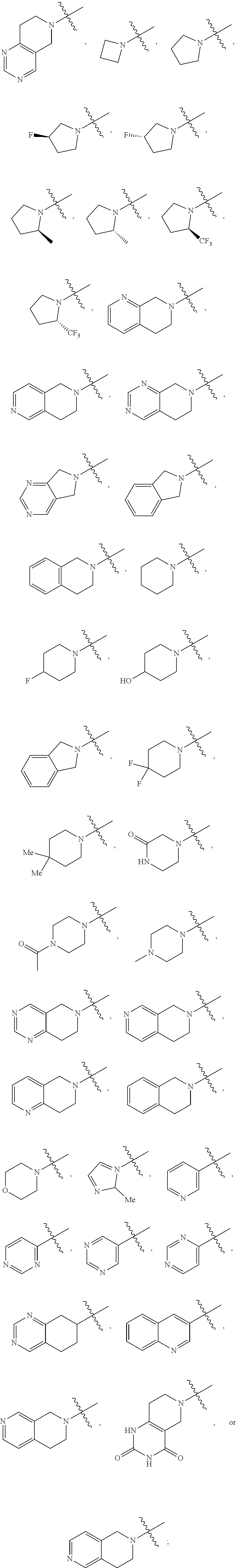
where [symbol] indicates a point of attachment.

33. The method of claim 29, wherein:
Lₐ is CH₂CH₂, CH₂CH₂CH₂, COCH₂, COCH₂CH₂, CH₂COCH₂;
L_{b} is absent,
L_{c} is N(C₁₋₆ alkyl), NH, N—C₁₋₆ alkyl, N—C₁₋₆ cycloalkyl, —NH—C₁₋₆ alkylene, —NH—C₁₋₆ alkylene-heteroarylene, —N(C₁₋₆ alkyl)-C₁₋₆ alkylene, —N—C₁₋₆ alkylene-cycloalkyl, N—C₁₋₆ alkylene-heterocycloalkyl, N—C₁₋₆ alkylene-aryl, or N—C₁₋₆ alkylene-heteroaryl; and
L_{d} is H, OH, C₁₋₆ alkyl, OH, alkoxy, NH₂, NHC₁₋₆ alkyl, N(C₁₋₆ alkyl)₂, C₁₋₆ alkyl, C₁₋₆ haloalkyl, optionally substituted cycloalkyl, optionally substituted heterocycloalkyl, optionally substituted aryl, or optionally substituted heteroaryl.

34. The method of claim 33, wherein:
LₐL_{b} is CH₂CH₂CH₂; and
L_{c}L_{d} is NH₂, NH-Me, NH-Et, NH-isopropyl, NH-cyclopropyl, NH-cyclobutyl, NH-cyclopentyl, N(Me)₂, N(Et)₂, N(Me)(Et), N(Me)-cyclopropyl, N(Me)-cyclobutyl, N(Me)-cyclopentyl, N(Me)CH₂-imidazolyl, N(Me)(iPr), N(Me)(tBu), NH-cyclopropyl, NH-oxazolyl, NH-pyrimidinyl, NH-pyridyl, NHCH₂-cyclopropyl, NHCH₂-oxazolyl, NHCH₂-pyrimidinyl, NHCH₂-pyridyl, NHCH₂-quinazolinyl, NHCH₂-quinolinyl, or NHCH₂-oxadiazolene-phenyl.

35. The method of claim 29, wherein:
Lₐ is CH₂CH₂CH₂;
L_{b} is absent;
L_{c} is absent; and
L_{d} is OH or alkoxy.

36. The method of claim 29, wherein:
Lₐ is CH₂CH₂CH₂,
L_{b} is absent; L_{c} is N(Me), N(Et), N(Me)(CH₂), or NH; and
L_{d} is H, methyl, ethyl, isopropyl, tert-butyl, cyclopropyl, cyclobutyl, cyclopentyl, cyclohexyl, pyrrolidinyl, oxazolyl, imidazolyl.

37. The method of claim 29, wherein:
Lₐ is CH₂CH₂CH₂;
L_{b} is absent;
L_{c} is CO, C(═O)O—, OC(═O)—NH, C(═O)NH, or C(═O)NHCH₂; and
L_{d} is H, C₁₋₆ alkyl, or optionally substuted aryl or heteroaryl.

38. The method of claim 29, wherein:
LₐL_{b} is CH₂CH₂CH₂; and
L_{c}L_{d} is O—C(═O)NH-phenyl, or

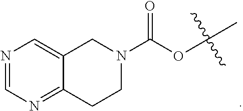 [SMILES: *OC(=O)N1CCc2ncncc2C1]

39. The method of claim 29, wherein:
Lₐ is CH₂CH₂CH₂:
L_{b} is optionally substituted cycloalkyl or heterocycloalkyl;
L_{c} is absent; and
L_{d} is H, OH, C₁₋₆ alkyl, C₁₋₆ haloalkyl, optionally substituted aryl or optionally substituted heteroaryl.

40. The method of claim 29, wherein:
Lₐ is CH₂CH₂CH₂;
L_{b} is absent;
L_{c} is absent; and
L_{d} is selected from the group consisting of, pyrrolidinyl, piperidinyl, piperazinyl,

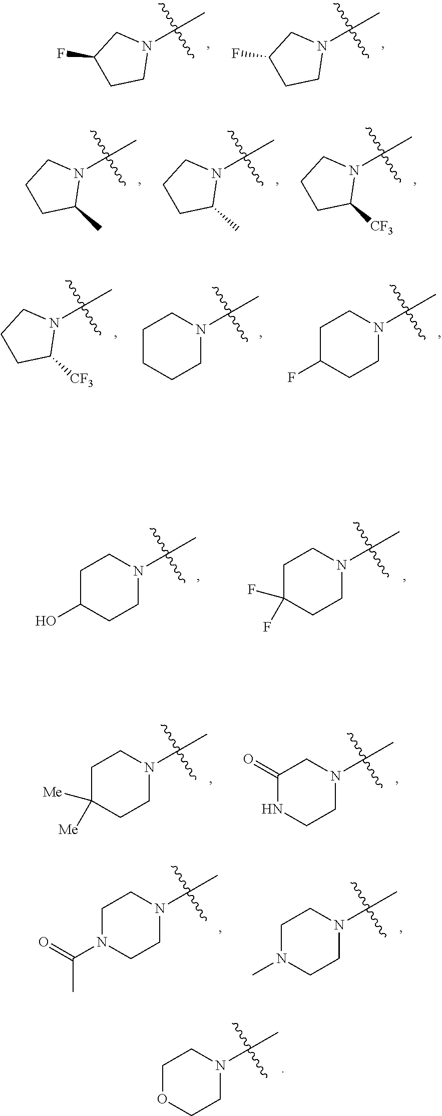

41. The method of claim 29, wherein:
Lₐ is CH₂CH₂CH₂;
L_{b} is absent;
L_{c} is absent; and
L_{d} is H, methyl, trifluoromethyl, phenyl, pyridyl, pyrimidinyl, OH, 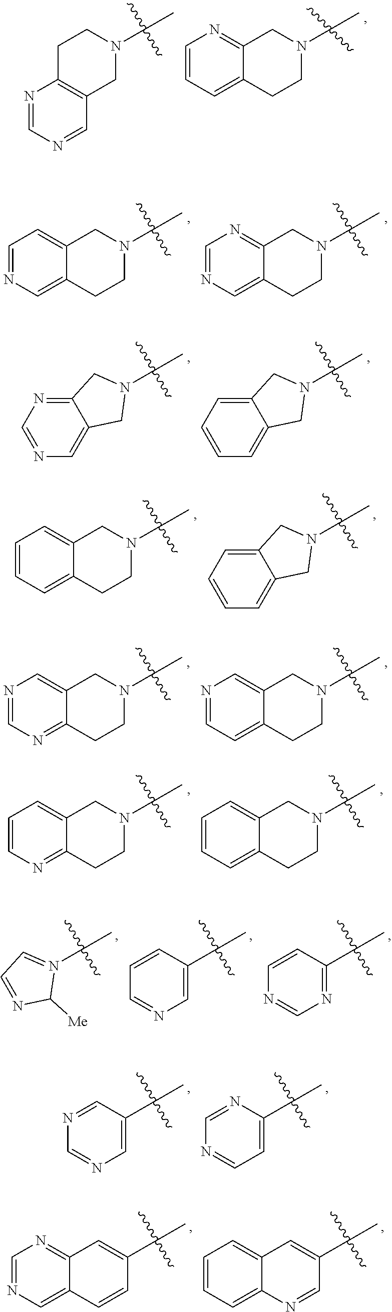 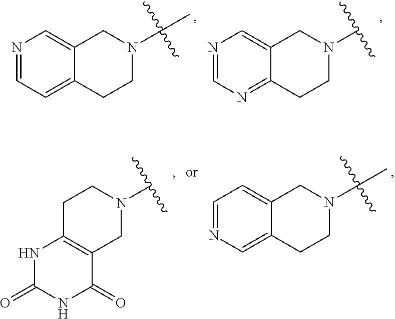 [SMILES: *N1CCc2ccncc2C1 ; *N1CCc2ncncc2C1 ; *N1CCc2[nH]c(=O)[nH]c(=O)c2C1 ; *N1CCc2cnccc2C1]
where [symbol] indicates a point of attachment.

42. The method of claim 29, wherein:
Lₐ is COCH₂CH₂;
L_{b} is absent;
L_{c} is absent; and
L_{d} is phenyl, pyridyl, pyrimidinyl, OH, 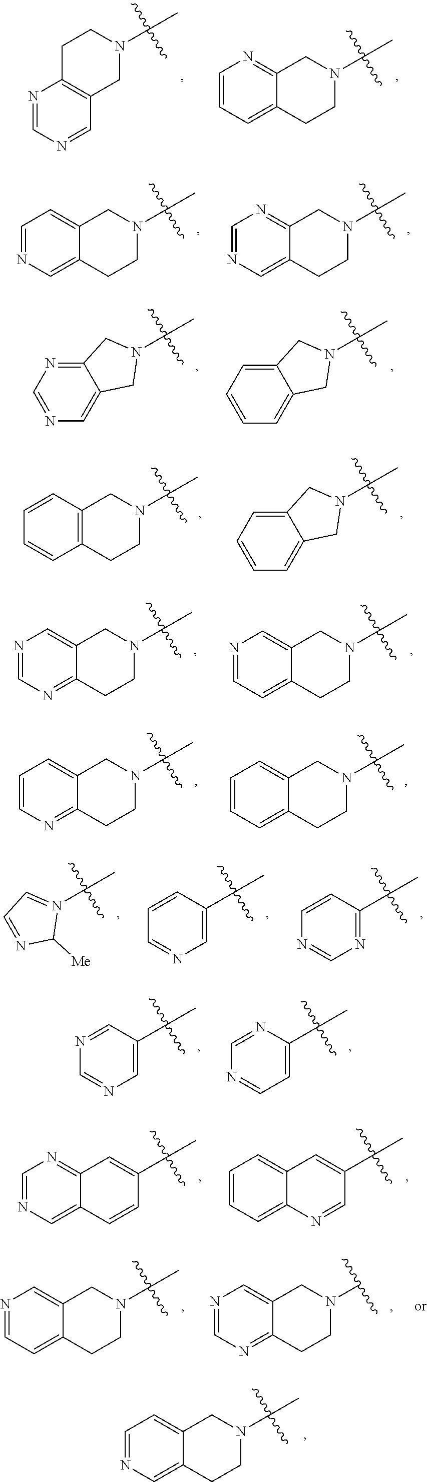
where [symbol] indicates a point of attachment.

43. The method of claim 29, wherein:
Lₐ is COCH₂CH₂;
L_{b} is absent;
L_{c} is C₁₋₆ alkylene, C₁₋₆ alkylene-N(C₁₋₆ alkyl), NH, N—C₁₋₆ alkyl, N—C₁₋₆ cycloalkyl, —NH—C₁₋₆ alkylene, —N(C₁₋₆ alkyl)-C₁₋₆ alkylene, —N—C₁₋₆ alkylene-cycloalkyl, N—C₁₋₆ alkylene-heterocycloalkyl, N—C₁₋₆ alkylene-aryl, N—C₁₋₆ alkylene-heteroaryl, C(═O), C(═O)O—, C(═O)NH, C(═O)N-alkyl, C(═O)NH—C₁₋₆ alkylene, C(═O)N(C₁₋₆ alkyl)-C₁₋₆ alkylene SO₂, SO₂ C₁₋₆ alkylene, SO₂NH, SO₂N—C₁₋₆ alkyl, OC(═O)—NH, OC(═O)—N-alkyl, SO₂, SO₂C₁₋₆ alkylene, SO₂NH, SO₂N(C₁₋₆ alkyl), SO₂NH—(C₁₋₆ alkylene, SO₂N(C₁₋₆ alkyl)-(C₁₋₆ alkylene), SO₂N(C₁₋₆ alkylene-aryl), SO₂N(C₁₋₆ alkylene-heteroaryl), 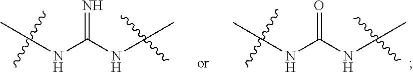 [SMILES: *NC(=N)N* ; *NC(=O)N*]
and
L_{d} is H, C₁₋₆ alkyl, OH, alkoxy, NH₂, NHC₁₋₆ alkyl, N(C₁₋₆ alkyl)₂, C₁₋₆ alkyl, C₁₋₆ haloalkyl, optionally substituted cycloalkyl, optionally substituted heterocycloalkyl optionally substituted aryl, optionally substituted heteroaryl.

44. The method of claim 29, wherein:
Lₐ is COCH₂CH₂;
L_{b} is absent;
L_{c} is NH or NHCH₂; and
L_{d} is optionally substituted cycloalkyl, optionally substituted heterocycloalkyl optionally substituted aryl, optionally substituted heteroaryl.

45. The method of claim 29, wherein:
Lₐ is COCH₂CH₂;
L_{b} is absent;
L_{c} is NH or NHCH₂; and
L_{d} is optionally substituted pyrimidinyl, optionally substituted quinolinyl, optionally substituted oxazolyl, optionally substituted cyclobutyl.

46. The method of claim 1 using a compound of formula C: 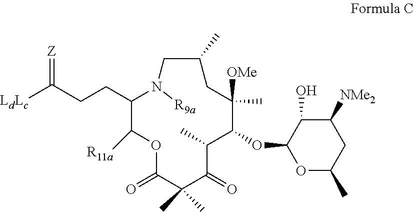
or a pharmaceutically acceptable salt thereof, wherein:
Z is H₂ or O;
R₉ₐ is selected from the group consisting of H, optionally substituted C₁₋₆ alkyl, C₁₋₆ alkylene-OH, C₁₋₆ alkylene-O—C₁₋₆ alkyl, C(═O)C₁₋₆ alkyl, C₁₋₆ alkylene-cyclolkyl, C(═O)cycloalkyl, or C(═O)NH-aryl;
L_{c} is absent or is C₁₋₄ alkylene, C₁₋₄ alkylene-N(C₁₋₄ alkyl), NH, N—C₁₋₄ alkyl, N—C₁₋₄ cycloalkyl, —NH—C₁₋₄ alkylene, —NH—C₁₋₆ alkylene-heteroarylene, —N(C₁₋₄ alkyl)-C₁₋₄ alkylene, —N—C₁₋₄ alkylene-cycloalkyl, N—C₁₋₄ alkylene-heterocycloalkyl, N—C₁₋₄ alkylene-aryl, N—C₁₋₄ alkylene-heteroaryl, C(═O), C(═O)O— C(═O)NH, C(═O)N-alkyl, C(═O)NH—C₁₋₄ alkylene, C(═O)N(C₁₋₄ alkyl)-C₁₋₄ alkylene SO₂, SO₂NH, SO₂N—C₁₋₄ alkyl, SO₂N—(C₁₋₄ alkyl)-(C₁₋₄ alkylene), OC(═O)—NH, OC(═O)—N—C₁₋₄ alkyl, SO₂, SO₂C₁₋₄ alkylene, SO₂NH, SO₂N(C₁₋₄ alkyl), SO₂NH—(C₁₋₄ alkylene, S)₂N(C₁₋₄ alkyl)-(C₁₋₄ alkylene), SO₂N(C₁₋₄ alkylene-aryl), SO₂N(C₁₋₄ alkylene-heteroaryl), 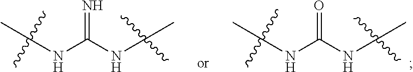 [SMILES: *NC(=N)N* ; *NC(=O)N*]
L_{d} is H, C₁₋₆ alkyl, OH, alkoxy, NH₂, NHC₁₋₆ alkyl, N(C₁₋₆ alkyl)₂, C₁₋₆ alkyl, C₁₋₆ haloalkyl, optionally substituted cycloalkyl, optionally substituted heterocycloalkyl optionally substituted aryl, optionally substituted heteroaryl, optionally substituted benzyl; and
R₁₁ₐ is H or C₁₋₆ alkyl.

47. The method of claim 46, wherein:
R₉ₐ is —H, methyl, ethyl, propyl, isopropyl, butyl, isobutyl, pentyl, isopentyl, acetyl, C(═O)—NH-phenyl, C(═O)-ethyl, C(═O)-cyclopropyl, CH₂-cyclobutyl, CH₂CH₂CH₂OH, CH₂CH₂CH₂OMe, or CH₂CH₂OH;
L_{c} is absent or is C₁₋₄ alkylene, C₁₋₄ alkylene-N(C₁₋₄ alkyl), NH, N(C₁₋₄ alkyl), N(C₁₋₄ cycloalkyl), —NH—C₁₋₄ alkylene, —N(C₁₋₄ alkyl)-C₁₋₄ alkylene, C(═O), C(═O)O—, C(═O)NH, C(═O)N-alkyl, C(═O)NH—C₁₋₄ alkylene, C(═O)N(C₁₋₄ alkyl)-C₁₋₄ alkylene, or OC(═O)—NH;
L_{d} is H, C₁₋₆ alkyl, OH, C₁₋₆ alkoxy, C₁₋₆ haloalkyl, optionally substituted C₃₋₆ cycloalkyl, optionally substituted 3-6 membered heterocycloalkyl, optionally substituted C₆₋₁₂ aryl, optionally substituted 5-10 membered heteroaryl.

48. The method of claim 46, wherein:
Z is H₂ or O;
R₉ₐ is —H, methyl, ethyl, propyl, isopropyl, butyl, isobutyl, isopentyl, acetyl, C(═O)—NH-phenyl, C(═O)-ethyl, C(═O)-cyclopropyl, CH₂-cyclobutyl, or CH₂CH₂OH; and
R₁₁ₐ is H or methyl.

49. The method of claim 1 using a compound of formula C1: 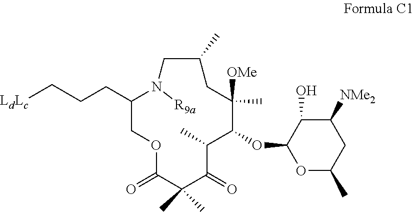
or a pharmaceutically acceptable salt thereof, wherein:
R₉ₐ is selected from the group consisting of H, C₁₋₆ alkyl, C₁₋₆ alkylene-OH, C₁₋₆ alkylene-O—C₁₋₆ alkyl, C(═O)C₁₋₆ alkyl, C₁₋₆ alkylene-cyclolkyl, C(═O)cycloalkyl, and C(═O)NH-aryl;
L_{c} is NH, NH—C₁₋₄ alkylene, NH—C₁₋₄ alkylene-(5-10 membered heteroarylene), C(═O)O—, C(═O)NH, or OC(═O)—NH; and
L_{d} is C₃₋₆ cycloalkyl, 6-10 membered aryl, or 5-10 memebred heteroaryl, wherein the cycloalkyl, aryl, and heteroaryl are each independently and optionally substituted with halo, C₁₋₄alkyl, C₁₋₄haloalkyl, or C₁₋₄alkoxy.

50. The method of claim 49, wherein
R₉ₐ is methyl, ethyl, propyl, isopropyl, butyl, isobutyl, pentyl, isopentyl, acetyl, C(═O)—NH-phenyl, C(═O)-ethyl, C(═O)-cyclopropyl, CH₂-cyclobutyl, CH₂CH₂CH₂OH, CH₂CH₂CH₂OMe, or CH₂CH₂OH;
L_{c} is NH, NH—CH₂, NH—CH₂-(5-6 membered heteroarylene), C(═O)O—, C(═O)NH, or OC(═O)—NH; and
L_{d} is C₃₋₆ cycloalkyl, phenyl, or 5-10 memebred heteroaryl.

51. The method of claim 1 using a compound of formula C2: 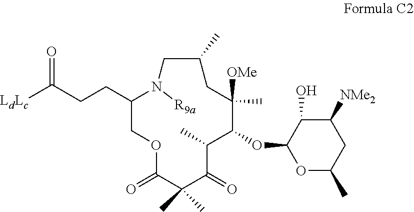
or a pharmaceutically acceptable salt thereof, wherein:
R₉ₐ is selected from the group consisting of H, substituted C₁₋₆ alkyl, C₁₋₆ alkylene-OH, C₁₋₆ alkylene-O—C₁₋₆ alkyl, C(═O)C₁₋₆ alkyl, C₁₋₆ alkylene-cyclolkyl, C(═O)cycloalkyl, and C(═O)NH-aryl;
L_{c} is absent or is NH, or NH—C₁₋₄ alkylene; and
L_{d} is OH, C₃₋₆ cycloalkyl, 5-10 membered heterocycloalkyl, 6-10 membered aryl, or 5-10 memebred heteroaryl, wherein the cycloalkyl, heterocycloalkyl, aryl, and heteroaryl are each independently and optionally substituted with halo, C₁₋₄alkyl, C₁₋₄haloalkyl, or C₁₋₄alkoxy.

52. The method of claim 51, wherein
R₉ₐ is methyl, ethyl, propyl, isopropyl, butyl, isobutyl, pentyl, isopentyl, acetyl, C(═O)—NH-phenyl, C(═O)-ethyl, C(═O)-cyclopropyl, CH₂-cyclobutyl, CH₂CH₂CH₂OH, CH₂CH₂CH₂OMe, or CH₂CH₂OH;
L_{c} is absent or is NH or NH—CH₂; and
L_{d} is OH, C₃₋₆ cycloalkyl, phenyl, 5-6 membered heteocycloalkyl, or 6-10 memebred nitrogen containing heteroaryl.

53. The method of claims 1-52, wherein the compound of formula I or a pharmaceutically acceptable salt thereof is selected from compounds depicted in Table A and Table B.

54. A compound or pharmaceutically acceptable salt thereof which is depicted in Table B.

55. A pharmaceutical composition comprising a compound of claim 54 or a pharmaceutically acceptable salt thereof and a pharmaceutically acceptable excipient.

56. The method claims 1-53, wherein the compound is admistered as a pharmaceutical composition comprising a pharmaceutically acceptable excipient.

57. The method of claims 1-53 wherein the genetic disease is associated with a premature termination codon mutation.

58. A method for treating a subject having a genetic disease is associated with a premature termination codon mutation, the method comprising:
administering an effective amount of a compound as recited in claim 1-53.

59. The method of any of claims 1-53, wherein the compound is administered to the subject alone or in any combination with an agent selected from the group consisting of aminoglycoside, potentiator, corrector, amplifier, and any combinations thereof.

60. The method of any of claims 1-53, wherein the genetic disease is selected from the group consisting of cystic fibrosis (CF), muscular dystrophy (Duchenne (DMD), Becker (BMD), congenital), spinal muscular atrophy (SMA), ataxia-telangiectasia, mucopolysaccharidosis type 1 (MPS1) (Hurler syndrome), hemophilia (A & B), Usher syndrome (Retinitis pigmentosa, X-linked retinitis pigmentosa), Tay-Sachs, factor VII deficiency, familial atrial fibrillation, Hailey-Hailey disease, McArdle disease, mucopolysaccharidosis, nephropathic cystinosis, polycystic kidney disease, Rett syndrome, cystinosis, severe epidermolysis bullosa, dravet syndrome, X-linked nephrogenic diabetes insipidus (XNDI), cancer, beta-thalassemia, obesity, epidermolysis bullosa (EB), and familial ademonous polypsis (FAP).

61. The method of any of claims 1-53 wherein the genetic disease is selected from the group consisting of cystic fibrosis (CF), muscular dystrophy (Duchenne (DMD), Becker (BMD), congenital), spinal muscular atrophy (SMA), hemophilia (A & B), Usher syndrome (Retinitis pigmentosa, X-linked retinitis pigmentosa), mucopolysaccharidosis, nephropathic cystinosis, Rett syndrome, cancer, Beta-thalassemia, obesity, epidermolysis bullosa (EB), and familial ademonous polypsis (FAP).

62. The method of any of claims 1-53 wherein the genetic disease is selected from the group consisting of cystic fibrosis, epidermolysis bullosa, severe epidermolysis bullosa, dystrophic epidermolysis bullosa, recessive dystrophic epidermolysis bullosa, junctional epidermolysis bullosa, and familial ademonous polypsis.

63. The method of 62, wherein the genetic disease is cystic fibrosis.

64. The method of 62, wherein the genetic disease is recessive dystrophic epidermolysis bullosa.

65. The method of 62, wherein the genetic disease is junctional epidermolysis bullosa.

66. The method of 62, wherein the genetic disease is familial ademonous polypsis.

67. A process for preparing a compound as defined in claims 1-53, comprising:
(a) coupling a compound of formula P-1 with a compound of formula P-4 to provide a compound of formula P-5. 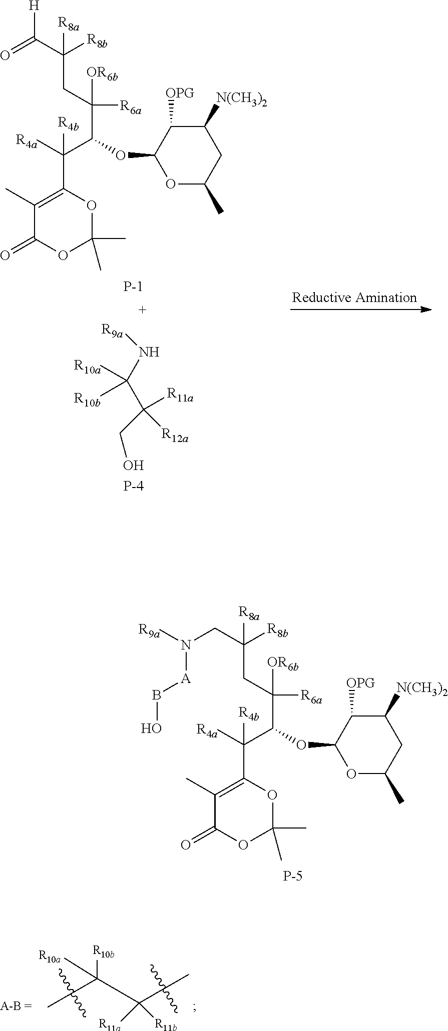
and
(b) cyclizing a compound of formula P-5 and subsequently deprotecting to provide a compound of formula I or a salt thereof: [chemical structure]
